(19) United States
(12) Patent Application Publication
(10) Pub. No.: US 2023/0049231 A1
(43) Pub. Date: Feb. 16, 2023

(54) ARYL HETEROBICYCLIC COMPOUNDS AS Kv1.3 POTASSIUM SHAKER CHANNEL BLOCKERS

(71) Applicant: D. E. Shaw Research, LLC, New York, NY (US)

(72) Inventors: Fabrizio GIORDANETTO, New York, NY (US); Morten Østergaard JENSEN, Copenhagen (DK); Vishwanath JOGINI, Hyderabad (IN); Roger John SNOW, Danbury, CT (US)

(21) Appl. No.: 17/766,834

(22) PCT Filed: Oct. 6, 2020
(86) PCT No.: PCT/US2020/054393

§ 371 Date: Apr. 6, 2022

Related U.S. Application Data
(60) Provisional application No. 62/911,648, filed on Oct. 7, 2019.

(51) Int. Cl. C07D 471/04; C07D 401/02; C07D 405/02; C07D 413/02; C07D 487/04

(52) U.S. Cl. CPC C07D 471/04; C07D 401/02; C07D 405/02; C07D 413/02; C07D 487/04

(57) ABSTRACT

A compound of Formula (I), or a pharmaceutically acceptable salt thereof, is described, where the substituents are as defined herein. Pharmaceutical compositions including the same and method of using the same are also described.

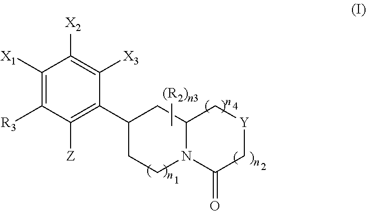

This application claims the benefit of and priority to U.S. Provisional Patent Application No. 62/911,648, filed on Oct. 7, 2019, the content of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### FIELD OF THE INVENTION

The invention relates generally to the field of pharmaceutical science. More particularly, the invention relates to compounds and compositions useful as pharmaceuticals as potassium channel blockers.

### BACKGROUND

Voltage-gated Kv1.3 potassium (K+) channels are expressed in lymphocytes (T and B lymphocytes), the central nervous system, and other tissues, and regulate a large number of physiological processes such as neurotransmitter release, heart rate, insulin secretion, and neuronal excitability. Kv1.3 channels can regulate membrane potential and thereby indirectly influence calcium signaling in human effector memory T cells (“TEMs”). TEMS are mediators of several conditions, including multiple sclerosis (“MS”), type I diabetes mellitus, psoriasis, spondylitis, parodontitis, and rheumatoid arthritis. Upon activation, TEMs increase expression of the Kv1.3 channel. Amongst human B cells, naive and early memory B cells express small numbers of Kv1.3 channels when they are quiescent. In contrast, class-switched memory B cells express high numbers of Kv1.3 channels. Furthermore, the Kv1.3 channel promotes the calcium homeostasis required for T-cell receptor-mediated cell activation, gene transcription, and proliferation (Panyi, G., et al., 2004, Trends Immunol., 565-569). Blockade of Kv1.3 channels in effector memory T cells suppresses activities like calcium signaling, cytokine production (e.g. interferon-gamma, interleukin 2), and cell proliferation.

Autoimmune disease is a family of disorders resulting from tissue damage caused by attack from the body's own immune system. Such diseases may affect a single organ, as in MS and type I diabetes mellitus, or may involve multiple organs, as in the case of rheumatoid arthritis and systemic lupus erythematosus. Treatment is generally palliative, with anti-inflammatory and immunosuppressive drugs, which can have severe side effects. A need for more effective therapies has led to a search for drugs that can selectively inhibit the function of TEMs, known to be involved in the etiology of autoimmune diseases. These inhibitors are thought to be able to ameliorate autoimmune disease symptoms without compromising the protective immune response. TEMs express high numbers of the Kv1.3 channel and depend on these channels for their function. In vivo, Kv1.3 channel blockers paralyze TEMs at the sites of inflammation and prevent their reactivation in inflamed tissues. Kv1.3 channel blockers do not affect the motility within lymph nodes of naive and central memory T cells. Suppressing the function of these cells by selectively blocking the Kv1.3 channel offers the potential for effective therapy of autoimmune diseases with minimal side effects.

MS is caused by autoimmune damage to the central nervous system (“CNS”). Symptoms include muscle weakness and paralysis, which severely affect quality of life for patients. MS progresses rapidly and unpredictably and eventually leads to death. The Kv1.3 channel is also highly expressed in auto-reactive TEMs from MS patients (Wulff H., et al., 2003, J. Clin. Invest., 1703-1713; Rus H., et al., 2005, PNAS, 11094-11099). Animal models of MS have been successfully treated using blockers of the Kv1.3 channel.

Compounds which are selective Kv1.3 channel blockers are thus potential therapeutic agents as immunosuppressants or immune system modulators. The Kv1.3 channel is also considered as a therapeutic target for the treatment of obesity and for enhancing peripheral insulin sensitivity in patients with type-2 diabetes mellitus. These compounds can also be utilized in the prevention of graft rejection and the treatment of immunological (e.g., autoimmune) and inflammatory disorders.

Tubulointerstitial fibrosis is a progressive connective tissue deposition on the kidney parenchyma, leading to renal function deterioration and is involved in the pathology of chronic kidney disease, chronic renal failure, nephritis, and inflammation in glomeruli, and is a common cause of end-stage renal failure. Overexpression of Kv1.3 channels in lymphocytes can promote their proliferation, leading to chronic inflammation and overstimulation of cellular immunity, which are involved in the underlying pathology of these renal diseases and are contributing factors in the progression of tubulointerstitial fibrosis. Inhibition of the lymphocyte Kv1.3 channel currents suppress proliferation of kidney lymphocytes and ameliorate the progression of renal fibrosis (Kazama I., et al., 2015, Mediators Inflamm., 1-12).

Kv1.3 channels also play a role in gastroenterological disorders including inflammatory bowel diseases (“IBDs”) such as ulcerative colitis (“UC”) and Crohn's disease. UC is a chronic IBD characterized by excessive T-cell infiltration and cytokine production. UC can impair quality of life and can lead to life-threatening complications. High levels of Kv1.3 channels in CD4 and CD8 positive T-cells in the inflamed mucosa of UC patients have been associated with production of pro-inflammatory compounds in active UC. Kv1.3 channels are thought to serve as a marker of disease activity and pharmacological blockade might constitute a novel immunosuppressive strategy in UC. Present treatment regimens for UC, including corticosteroids, salicylates, and anti-TNF-α reagents, are insufficient for many patients (Hansen L. K., et al., 2014, J. Crohns Colitis, 1378-1391). Crohn's disease is a type of IBD which may affect any part of the gastrointestinal tract. Crohn's disease is thought to be the result of intestinal inflammation due to a T-cell-driven process initiated by normally safe bacteria. Thus, Kv1.3 channel inhibition can be utilized in treating the Crohn's disease.

In addition to T cells, Kv1.3 channels are also expressed in microglia, where the channel is involved in inflammatory cytokine and nitric oxide production and in microglia-mediated neuronal killing. In humans, strong Kv1.3 channel expression has been found in microglia in the frontal cortex of patients with Alzheimer's disease and on CD68⁺ cells in multiple sclerosis brain lesions. It has been suggested that Kv1.3 channel blockers might be able to preferentially target detrimental proinflammatory microglia functions. Kv1.3 channels are expressed on activated microglia in infarcted rodent and human brain. Higher Kv1.3 channel current densities are observed in acutely isolated microglia from the infarcted hemisphere than in microglia isolated from the contralateral hemisphere of a mouse model of stroke (Chen Y. J., et al., 2017, Ann. Clin. Transl. Neurol., 147-161).

Expression of Kv1.3 channels is elevated in microglia of human Alzheimer's disease brains, suggesting that Kv1.3 channel is a pathologically relevant microglial target in Alzheimer's disease (Rangaraju S., et al., 2015, J. Alzheimers Dis., 797-808). Soluble AβO enhances microglial Kv1.3 channel activity. Kv1.3 channels are required for AβO-induced microglial pro-inflammatory activation and neurotoxicity. Kv1.3 channel expression/activity is upregulated in transgenic Alzheimer's disease animals and human Alzheimer's disease brains. Pharmacological targeting of microglial Kv1.3 channels can affect hippocampal synaptic plasticity and reduce amyloid deposition in APP/PS1 mice. Thus, Kv1.3 channel may be a therapeutic target for Alzheimer's disease.

Kv1.3 channel blockers could be also useful for ameliorating pathology in cardiovascular disorders such as ischemic stroke, where activated microglia significantly contribute to the secondary expansion of the infarct.

Kv1.3 channel expression is associated with the control of proliferation in multiple cell types, apoptosis, and cell survival. These processes are crucial for cancer progression. In this context, Kv1.3 channels located in the inner mitochondrial membrane can interact with the apoptosis regulator Bax (Serrano-Albarras, A., et al., 2018, Expert Opin. Ther. Targets, 101-105). Thus, inhibitors of Kv1.3 channels may be used as anticancer agents.

A number of peptide toxins with multiple disulfide bonds from spiders, scorpions, and anemones are known to block Kv1.3 channels. A few selective, potent peptide inhibitors of the Kv1.3 channel have been developed. A synthetic derivative of stichodactyla toxin (“shk”) with an unnatural amino acid (shk-186) is the most advanced peptide toxin. Shk has demonstrated efficacy in preclinical models and is currently in a phase I clinical trial for treatment of psoriasis. Shk can suppress proliferation of TEMs, resulting in improved condition in animal models of MS. Unfortunately, Shk also binds to the closely-related Kvi channel subtype found in CNS and heart. There is a need for Kv1.3 channel-selective inhibitors to avoid potential cardio- and neuro-toxicity. Additionally, small peptides like shk-186 are rapidly cleared from the body after administration, resulting in short circulating half-lives and frequent administration events. Thus, there is a need for the development of long-acting, selective Kv1.3 channel inhibitors for the treatment of chronic inflammatory diseases.

Thus, there remains a need for development of novel Kv1.3 channel blockers as pharmaceutical agents.

### SUMMARY OF THE INVENTION

In one aspect, compounds useful as potassium channel blockers having a structure of Formula I

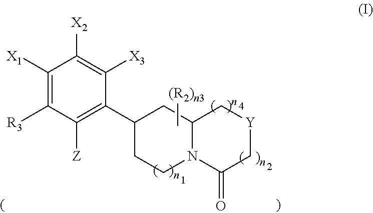

are described, where the various substituents are defined herein. The compounds of Formula I described herein can block Kv1.3 potassium (K+) channels and be used in the treatment of a variety of conditions. Methods for synthesizing these compounds are also described herein. Pharmaceutical compositions and methods of using these compositions described herein are useful for treating conditions in vitro and in vivo. Such compounds, pharmaceutical compositions, and methods of treatment have a number of clinical applications, including as pharmaceutically active agents and methods for treating cancer, an immunological disorder, a Central Nerve System (CNS) disorder, an inflammatory disorder, a gastroenterological disorder, a metabolic disorder, a cardiovascular disorder, a kidney disease, or a combination thereof.

In one aspect, a compound of Formula I or a pharmaceutically acceptable salt thereof is described,

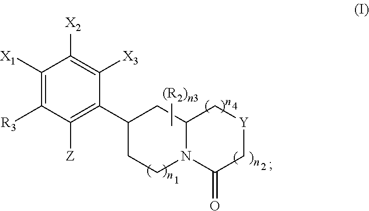

where

Y is C(R₂)₂, NR₁, or O;

Z is ORₐ;

X₁ is H, halogen, or alkyl;

X₂ is H, halogen, CN, alkyl, cycloalkyl, halogenated cycloalkyl, or halogenated alkyl;

X₃ is H, halogen, halogenated alkyl, or alkyl;

or alternatively X₁ and X₂ and the carbon atoms they are connected to taken together form an optionally substituted 5- or 6-membered aryl;

or alternatively X₂ and X₃ and the carbon atoms they are connected to taken together form an optionally substituted 5- or 6-membered aryl;

each occurrence of R₁ is independently H, alkyl, alkenyl, cycloalkyl, heteroalkyl, cycloheteroalkyl, aryl, heteroaryl, (CR₆R₇)ₙ₆ORₐ, (CR₆R₇)ₙ₆N(Rₐ)₂, (C═O)Rₐ, (C═O)ORₐ, (CR₆R₇)ₙ₆(C═O)NRₐR_{b}, SO₂Rₐ or (CR₆R₇)ₙ₆-heterocycle;

each occurrence of R₂ is independently H, halogen, CN, alkyl, cycloalkyl, heteroalkyl, cycloheteroalkyl, (CR₆R₇)ₙ₆ORₐ, (CR₆R₇)ₙ₆-heterocycle, (C═O)ORₐ, (CR₆R₇)ₙ₆NRₐ(C═O)Rₐ, (CR₆R₇)ₙ₆N(Rₐ)₂, NRₐ(CR₆R₇)ₙ₆ORₐ, (C═O)NRₐ(CR₆R₇)ₙ₆ORₐ, (C═O)Rₐ, (CR₆R₇)ₙ₆(C═O)NRₐR_{b}, aryl, or heteroaryl, wherein each R₂ may be attached to any one of the carbon ring atoms of

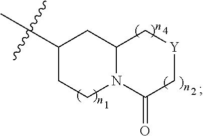

R₃ is H, alkyl, or halogen;

each occurrence of R₆ and R₇ are independently H, alkyl, cycloalkyl, optionally substituted aryl, or optionally substituted heteroaryl;

each occurrence of Rₐ and R_{b} are independently H, alkyl, alkenyl, cycloalkyl, saturated heterocycle, aryl, or heteroaryl; or alternatively Rₐ and R_{b} together with the nitrogen atom that they are connected to form an optionally substituted heterocycle;

the heterocycle comprises 1-3 heteroatoms each selected from the group consisting of N, O, and S;

the alkyl, cycloalkyl, heteroalkyl, cycloheteroalkyl, heterocycle, aryl, and heteroaryl in X₁, X₂, X₃, R₁, R₂, R₃, R₆, R₇, Rₐ, and R_{b}, where applicable, are each independently and optionally substituted by 1-4 substituents each independently selected from the group consisting of alkyl, cycloalkyl, halogenated alkyl, halogenated cycloalkyl, halogen, CN, R₈, OR₈, —(CH₂)₁₋₂OR₈, N(R₈)₂, (C═O)R₈, (C═O)N(R₈)₂, NR₈(C═O)R₈, and oxo where valence permits;

each occurrence of R₈ is independently H, alkyl, cycloalkyl, or a heterocycle optionally substituted by alkyl; or alternatively the two R₈ groups together with the nitrogen atom that they are connected to form a heterocycle optionally substituted by alkyl and comprising the nitrogen atom and 0-3 additional heteroatoms each selected from the group consisting of N, O, and S;

n₁ is an integer from 0-1;

n₂ is an integer from 0-2;

n₃ is an integer from 0-3;

n₄ is an integer from 1 to 2; and

n₆ is an integer from 0-3.

In any one of the embodiments described herein, the structural moiety

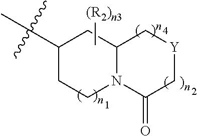

has the structure of

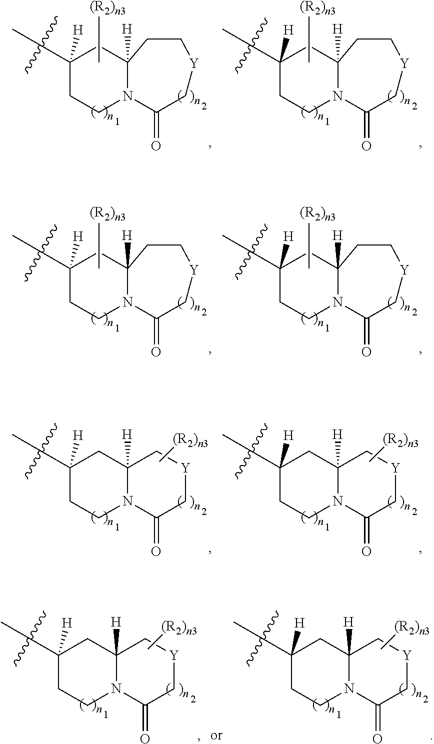

In any one of the embodiments described herein, the structural moiety

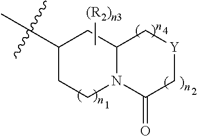

has the structure of

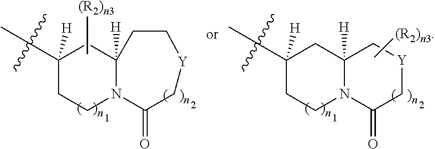

In any one of the embodiments described herein, the structural moiety

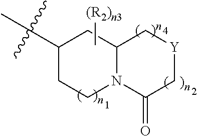

has the structure of

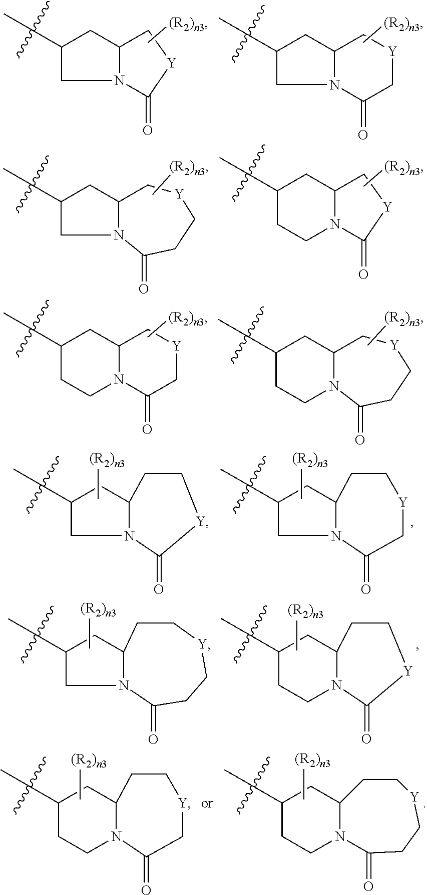

In any one of the embodiments described herein, the structural moiety

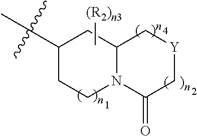

has the structure of

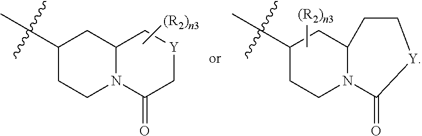

In any one of the embodiments described herein, the structural moiety

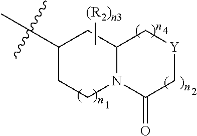

has the structure of

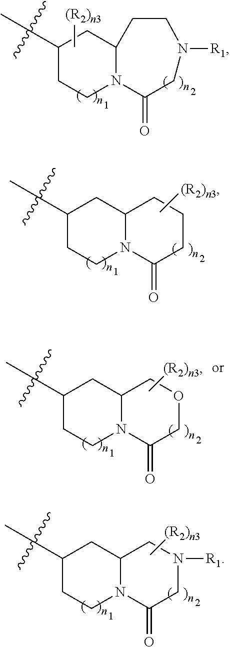

In any one of the embodiments described herein, wherein the structural moiety

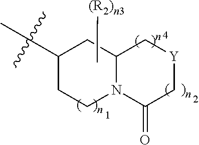

has the structure of

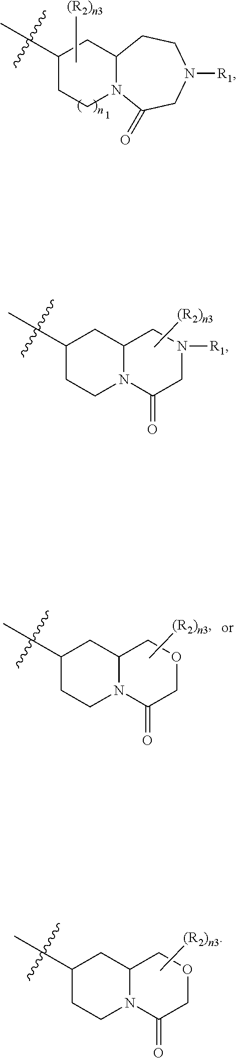

In any one of the embodiments described herein, the structural moiety

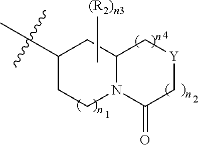

has the structure of

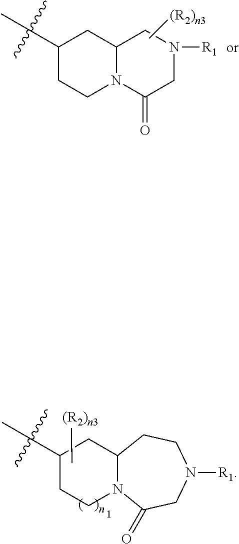

In any one of the embodiments described herein, R₁ is H, alkyl, alkenyl, cycloalkyl, heteroalkyl, or cycloheteroalkyl.

In any one of the embodiments described herein, R₁ is aryl or heteroaryl.

In any one of the embodiments described herein, R₁ is (C═O)Rₐ, (C═O)ORₐ, SO₂Rₐ, (CR₆R₇)ₙ₆ORₐ, (CR₆R₇)ₙ₆N(Rₐ)₂, (CR₆R₇)ₙ₆(C═O)NRₐR_{b}, or (CR₆R₇)ₙ₆-heterocycle.

In any one of the embodiments described herein, R₁ is (C═O)Rₐ.

In any one of the embodiments described herein, Rₐ and R_{b} are each independently H, alkyl, or alkyl substituted by one or more OR₈.

In any one of the embodiments described herein, R₈ is H or alkyl.

In any one of the embodiments described herein, R₁ is selected from the group consisting of H, —CH₃, —(CH₂)₂OH, —(CH₂)₂NH₂, —CONH₂, —CONHMe, —CONMe₂, —CONEt₂, SO₂Me, and SO₂Et.

In any one of the embodiments described herein, R₁ is selected from the group consisting of

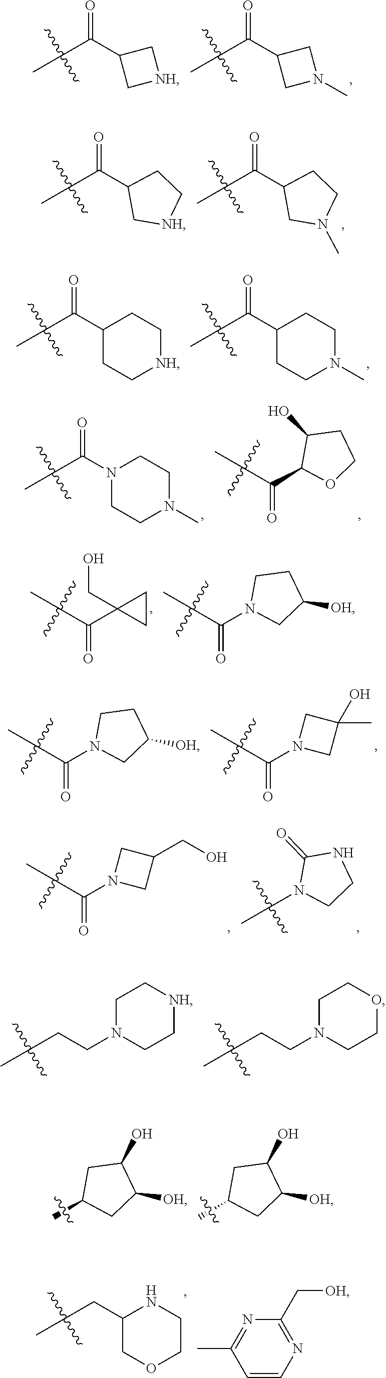

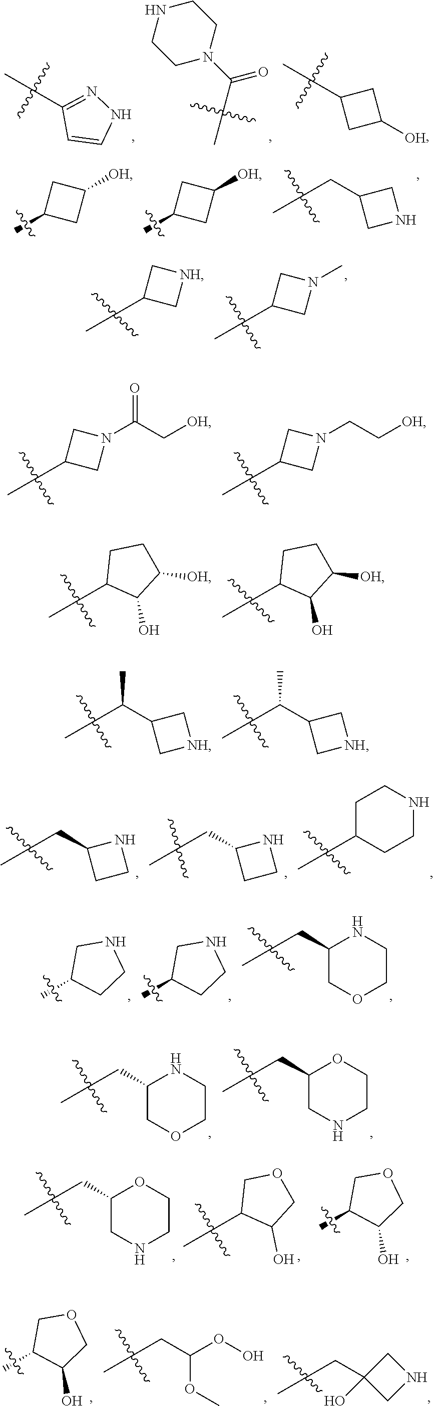

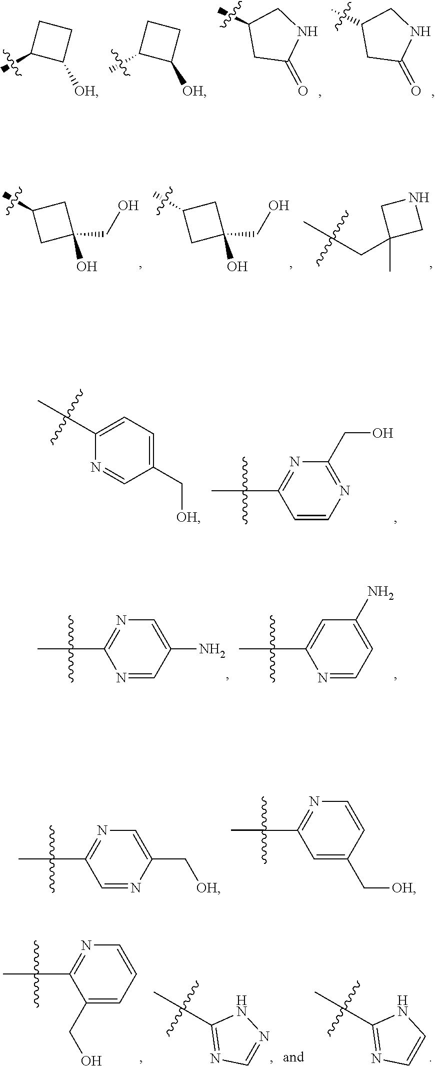

In any one of the embodiments described herein, R₁ is selected from the group consisting of

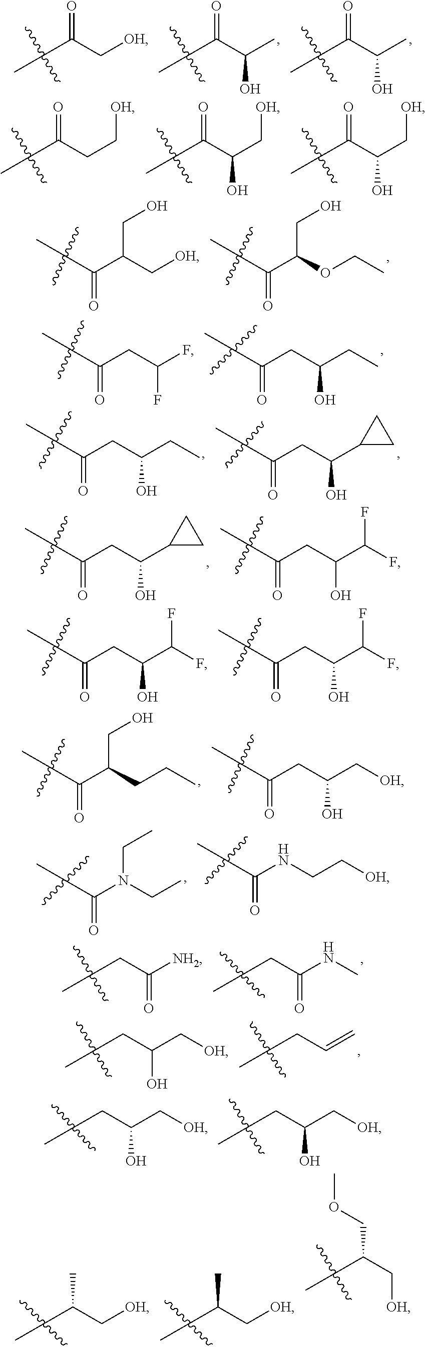

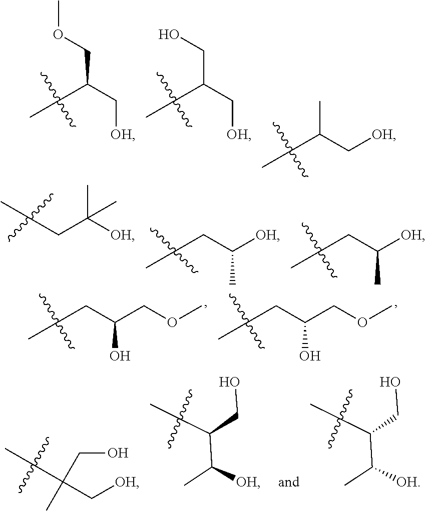

In any one of the embodiments described herein, at least one occurrence of R₂ is H, halogen, CN, alkyl, heteroalkyl, cycloalkyl, cycloheteroalkyl, ORₐ, N(R₁)₂, (C═O)Rₐ, (C═O)NRₐR_{b}, aryl, or heteroaryl.

In any one of the embodiments described herein, at least one occurrence of R₂ is (CR₆R₇)ₙ₆ORₐ, (CR₆R₇)ₙ₆-heterocycle, (C═O)Rₐ, (C═O)ORₐ, (CR₆R₇)ₙ₆NRₐ(C═O)Rₐ, (CR₆R₇)ₙ₆N(Rₐ)₂, NRₐ(CR₆R₇)ₙ₆ORₐ, (C═O)NRₐ(CR₆R₇)ₙ₆ORₐ, or (CR₆R₇)ₙ₆(C═O)NRₐR_{b}.

In any one of the embodiments described herein, at least one occurrence of R₂ is CH₃, —CH₂—OH, —CH₂—CH₂—OH, —CH(OH)—CH₃, —CH₂—NH₂,

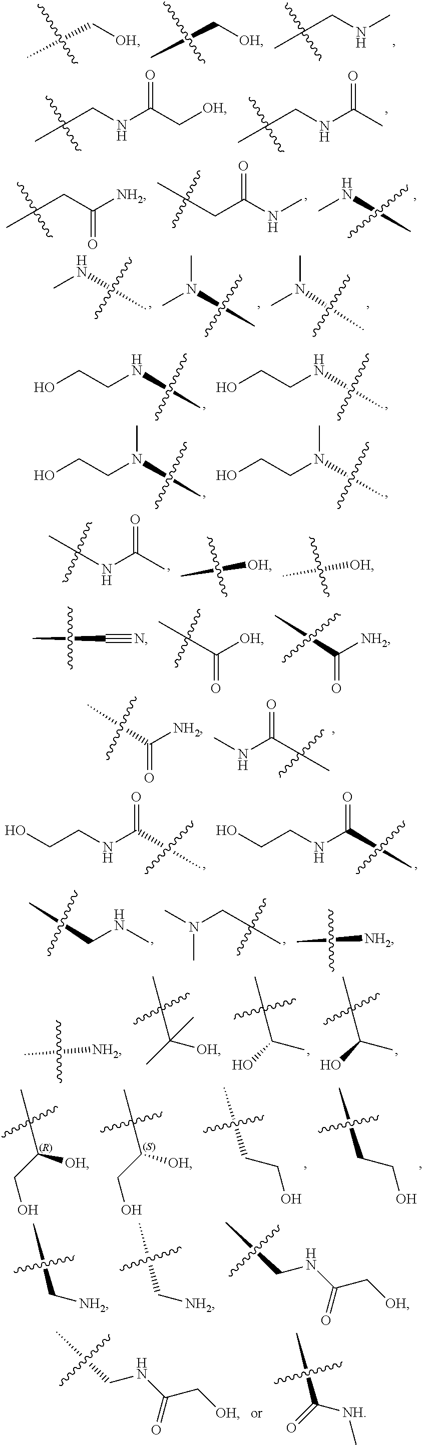

In any one of the embodiments described herein, at least one occurrence of R₂ is heteroalkyl, cycloheteroalkyl,

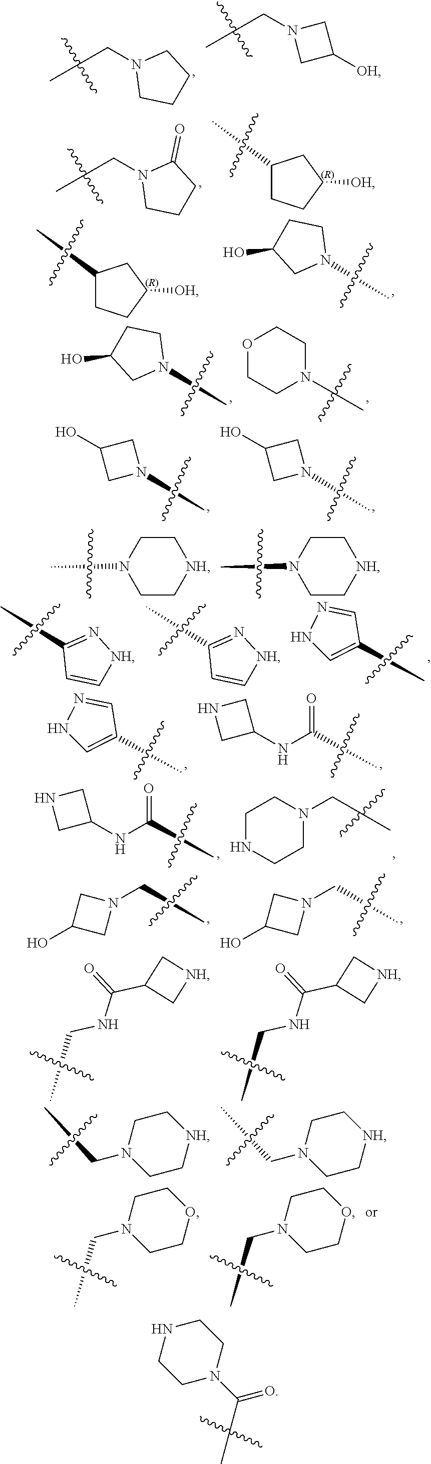

In any one of the embodiments described herein, n₁ is 0.

In any one of the embodiments described herein, n₁ is 1.

In any one of the embodiments described herein, n₂ is 0 or 1.

In any one of the embodiments described herein, n₃ is 0, 1, or 2.

In any one of the embodiments described herein, n₄ is 1.

In any one of the embodiments described herein, n₆ is 0, 1, or 2.

In any one of the embodiments described herein, Z is OH, OMe, OEt, OPr, O-i-Pr, O-t-Bu, O-iso-Bu, O-sec-Bu, or OBu.

In any one of the embodiments described herein, Z is OH, OMe, or OEt.

In any one of the embodiments described herein, Z is OH.

In any one of the embodiments described herein, X₁ is H, halogen, Me, or Et.

In any one of the embodiments described herein, X₁ is H, F, Cl, Br, or Me.

In any one of the embodiments described herein, X₁ is H or Cl.

In any one of the embodiments described herein, X₂ is H, halogen, fluorinated alkyl, or alkyl.

In any one of the embodiments described herein, X₂ is H, F, Cl, Br, Me, CF₂H, CF₂Cl, or CF₃.

In any one of the embodiments described herein, X₂ is H or Cl.

In any one of the embodiments described herein, X₃ is H, F, Cl, Br, Me, CF₂H, CF₂Cl, or CF₃.

In any one of the embodiments described herein, X₃ is H or Cl.

In any one of the embodiments described herein, R₃ is H.

In any one of the embodiments described herein, R₃ is alkyl.

In any one of the embodiments described herein, R₃ is halogen.

In any one of the embodiments described herein, R₃ is H, F, Cl, or Me.

In any one of the embodiments described herein, the structural moiety

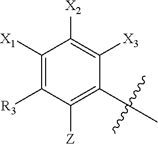

has the structure of

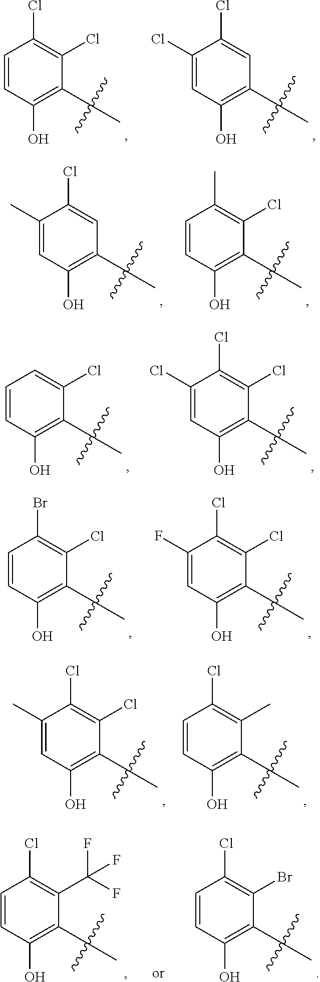

In any one of the embodiments described herein, the compound has a structure of Formula II′ or II:

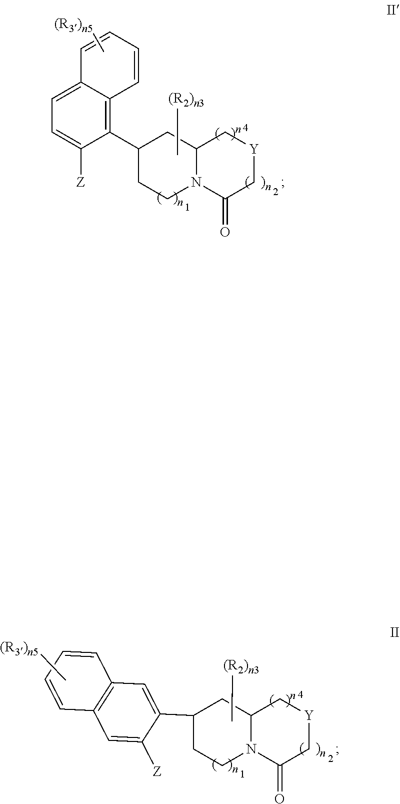

wherein R_{3′} is independently H, halogen, or alkyl; and

n₅ is an integer from 0-3.

In any one of the embodiments described herein, n₅ is 0, 1, or 2.

In any one of the embodiments described herein, n₅ is 0.

In any one of the embodiments described herein, R_{3′} is H or alkyl.

In any one of the embodiments described herein, R_{3′} is halogen.

In any one of the embodiments described herein, Z is OH, OMe, OEt, OPr, O-i-Pr, O-t-Bu, O-iso-Bu, O-sec-Bu, or OBu.

In any one of the embodiments described herein, Z is OH, OMe, or OEt.

In any one of the embodiments described herein, Z is OH.

In any one of the embodiments described herein, at least one occurrence of Rₐ or R_{b} is independently H, alkyl, cycloalkyl, saturated heterocycle, aryl, or heteroaryl.

In any one of the embodiments described herein, at least one occurrence of Rₐ or R_{b} is independently H, Me Et, Pr, or a heterocycle selected from the group consisting of

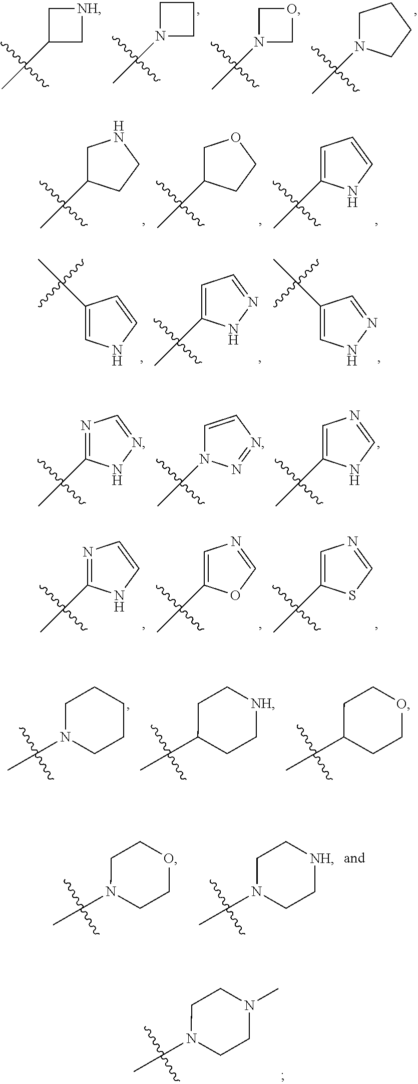

wherein the heterocycle is optionally substituted by alkyl, OH, oxo, or (C═O)C₁₋₄alkyl where valence permits.

In any one of the embodiments described herein, Rₐ and R_{b} together with the nitrogen atom that they are connected to form an optionally substituted heterocycle comprising the nitrogen atom and 0-3 additional heteroatoms each selected from the group consisting of N, O, and S.

In any one of the embodiments described herein, the heterocycle is selected from the group consisting of

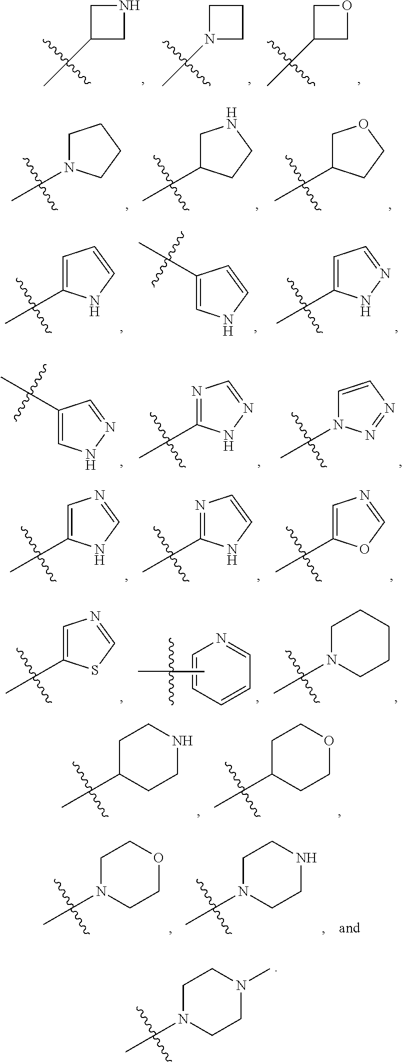

In any one of the embodiments described herein, the compound is selected from the group consisting of compounds 1-62 as shown in Table 4.

In any one of the embodiments described herein, the compound is selected from the group consisting of compounds 63-78, 83-85, 87-88, 90-94, 96-97, 99-104, 109-176, 180-208, 213-220, 223-293 as shown in Table 5.

In another aspect, a pharmaceutical composition is described, including at least one compound according to any one of the embodiments described herein or a pharmaceutically acceptable salt thereof and a pharmaceutically acceptable carrier or diluent.

In yet another aspect, a method of treating a condition in a mammalian species in need thereof is described, including administering to the mammalian species a therapeutically effective amount of at least one compound according to any one of the embodiments described herein or a pharmaceutically acceptable salt thereof, wherein the condition is selected from the group consisting of cancer, an immunological disorder, a Central Nerve System (CNS) disorder, an inflammatory disorder, a gastroenterological disorder, a metabolic disorder, a cardiovascular disorder, and a kidney disease.

In any one of the embodiments described herein, the immunological disorder is transplant rejection or an autoimmune disease.

In any one of the embodiments described herein, the autoimmune disease is rheumatoid arthritis, multiple sclerosis, systemic lupus erythematosus, or type I diabetes mellitus.

In any one of the embodiments described herein, the central nervous system disorder is Alzheimer's disease.

In any one of the embodiments described herein, the inflammatory disorder is an inflammatory skin condition, arthritis, psoriasis, spondylitis, parodontitits, or an inflammatory neuropathy.

In any one of the embodiments described herein, the gastroenterological disorder is an inflammatory bowel disease.

In any one of the embodiments described herein, the metabolic disorder is obesity or type II diabetes mellitus.

In any one of the embodiments described herein, the cardiovascular disorder is an ischemic stroke.

In any one of the embodiments described herein, the kidney disease is chronic kidney disease, nephritis, or chronic renal failure.

In any one of the embodiments described herein, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

In any one of the embodiments described herein, the mammalian species is human.

In yet another aspect, a method of blocking Kv1.3 potassium channel in a mammalian species in need thereof is described, including administering to the mammalian species a therapeutically effective amount of at least one compound according to any one of the embodiments described herein or a pharmaceutically acceptable salt thereof.

In any one of the embodiments described herein, the mammalian species is human.

Any one of the embodiments disclosed herein may be properly combined with any other embodiment disclosed herein. The combination of any one of the embodiments disclosed herein with any other embodiments disclosed herein is expressly contemplated. Specifically, the selection of one or more embodiments for one substituent group can be properly combined with the selection of one or more particular embodiments for any other substituent group. Such combination can be made in any one or more embodiments of the application described herein or any formula described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The terms “alkyl” and “alk” refer to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. “Substituted alkyl” refers to an alkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle, and each occurrence of Rₑ is independently alkyl, cycloalkyl, alkenyl, cycloalkenyl, alkynyl, heterocycle, or aryl. In some embodiments, groups such as alkyl, cycloalkyl, alkenyl, alkynyl, cycloalkenyl, heterocycle, and aryl can themselves be optionally substituted.

The term “heteroalkyl” refers to a straight- or branched-chain alkyl group preferably having from 2 to 12 carbons, more preferably 2 to 10 carbons in the chain, one or more of which has been replaced by a heteroatom selected from the group consisting of S, O, P, and N. Exemplary heteroalkyls include, but are not limited to, alkyl ethers, secondary and tertiary alkyl amines, alkyl sulfides, and the like. The group may be a terminal group or a bridging group.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methyl-(E)-but-2-enyl, 2-methyl-(Z)-but-2-enyl, 2,3-dimethyl-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl, (Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include, but are not limited to, one or more of the following groups: hydrogen, halogen, alkyl, halogenated alkyl (i.e., an alkyl group bearing a single halogen substituent or multiple halogen substituents such as CF₃ or CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, or hex-3-ynyl. “Substituted alkynyl” refers to an alkynyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally to form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “heterocycloalkyl” or “cycloheteroalkyl” refers to a saturated or partially saturated monocyclic, bicyclic, or polycyclic ring containing at least one heteroatom selected from the group consisting of nitrogen, sulfur, and oxygen, preferably from 1 to 3 heteroatoms in at least one ring. Each ring is preferably from 3 to 10 membered, more preferably 4 to 7 membered. Examples of suitable heterocycloalkyl substituents include, but are not limited to, pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane. The group may be a terminal group or a bridging group.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NRₐS(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c}, and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl, or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The term “fused aromatic ring” refers to a molecular structure having two or more aromatic rings where two adjacent aromatic rings have two carbon atoms in common. “Substituted aryl” refers to an aryl group substituted by one or more substituents, preferably 1 to 3 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)ORₐ, C(═O)Rₐ, C(═O)NR_{b}R_{c}, OC(═O)Rₐ, OC(═O)NR_{b}R_{c}, NR_{b}C(═O)ORₑ, NRₐC(═O)NR_{b}R_{c}, NR_{d}S(═O)₂NR_{b}R_{c}, NRₐP(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cyclic groups, especially fused cycloalkyl, fused cycloalkenyl, fused heterocycle, or fused aryl, where the aforementioned cycloalkyl, cycloalkenyl, heterocycle, and aryl substituents can themselves be optionally substituted.

The term “biaryl” refers to two aryl groups linked by a single bond. The term “biheteroaryl” refers to two heteroaryl groups linked by a single bond. Similarly, the term “heteroaryl-aryl” refers to a heteroaryl group and an aryl group linked by a single bond and the term “aryl-heteroaryl” refers to an aryl group and a heteroaryl group linked by a single bond. In certain embodiments, the numbers of the ring atoms in the heteroaryl and/or aryl rings are used to specify the sizes of the aryl or heteroaryl ring in the substituents. For example, 5,6-heteroaryl-aryl refers to a substituent in which a 5-membered heteroaryl is linked to a 6-membered aryl group. Other combinations and ring sizes can be similarly specified.

The term “carbocycle” or “carbon cycle” refers to a fully saturated or partially saturated cyclic hydrocarbon group containing from 1 to 4 rings and 3 to 8 carbons per ring, or cyclic, aromatic hydrocarbon groups that have 1 to 5 aromatic rings, especially monocyclic or bicyclic groups such as phenyl, biphenyl, or naphthyl. The term “carbocycle” encompasses cycloalkyl, cycloalkenyl, cycloalkynyl, and aryl as defined hereinabove. The term “substituted carbocycle” refers to carbocycle or carbocyclic groups substituted with one or more substituents, preferably 1 to 4 substituents, at any available point of attachment. Exemplary substituents include, but are not limited to, those described above for substituted cycloalkyl, substituted cycloalkenyl, substituted cycloalkynyl, and substituted aryl. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle, and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 3 to 7 membered monocyclic, 7 to 11 membered bicyclic, or 8 to 16 membered tricyclic ring systems) which have at least one heteroatom in at least one carbon atom-containing ring. Each ring of the heterocyclic group may independently be saturated, or partially or fully unsaturated. Each ring of the heterocyclic group containing a heteroatom may have 1, 2, 3, or 4 heteroatoms selected from the group consisting of nitrogen atoms, oxygen atoms, and sulfur atoms, where the nitrogen and sulfur heteroatoms may optionally be oxidized and the nitrogen heteroatoms may optionally be quaternized. The term “heteroarylium” refers to a heteroaryl group bearing a quaternary nitrogen atom and thus a positive charge. The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane, tetrahydro-1,1-dioxothienyl, and the like. Exemplary bicyclic heterocyclic groups include indolyl, indolinyl, isoindolyl, benzothiazolyl, benzoxazolyl, benzoxadiazolyl, benzothienyl, benzo[d][1,3]dioxolyl, dihydro-2H-benzo[b][1,4]oxazine, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, dihydrobenzo[d]oxazole, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl, and the like. Exemplary tricyclic heterocyclic groups include carbazolyl, benzidolyl, phenanthrolinyl, acridinyl, phenanthridinyl, xanthenyl, and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cyclic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “oxo” refers to the

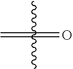 [SMILES: *=O]

substituent group, which may be attached to a carbon ring atom on a carboncycle or heterocycle. When an oxo substituent group is attached to a carbon ring atom on an aromatic group, e.g., aryl or heteroaryl, the bonds on the aromatic ring may be rearranged to satisfy the valence requirement. For instance, a pyridine with a 2-oxo substituent group may have the structure of

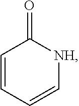

which also includes its tautomeric form of

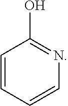

The term “alkylamino” refers to a group having the structure —NHR′, where R′ is hydrogen, alkyl or substituted alkyl, or cycloalkyl or substituted cycloalkyl, as defined herein. Examples of alkylamino groups include, but are not limited to, methylamino, ethylamino, n-propylamino, iso-propylamino, cyclopropylamino, n-butylamino, t-butylamino, neopentylamino, n-pentylamino, hexylamino, cyclohexylamino, and the like.

The term “dialkylamino” refers to a group having the structure —NRR′, where R and R′ are each independently alkyl or substituted alkyl, cycloalkyl or substituted cycloalkyl, cycloalkenyl or substituted cyclolalkenyl, aryl or substituted aryl, or heterocycle or substituted heterocycle, as defined herein. R and R′ may be the same or different in a dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(t-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of the resulting cyclic structure include, but are not limited to, aziridinyl, pyrrolidinyl, piperidinyl, morpholinyl, pyrrolyl, imidazolyl, 1,2,4-triazolyl, and tetrazolyl.

The terms “halogen” or “halo” refer to chlorine, bromine, fluorine, or iodine.

The term “substituted” refers to the embodiments in which a molecule, molecular moiety, or substituent group (e.g., alkyl, cycloalkyl, alkenyl, cycloalkenyl, alkynyl, heterocycle, or aryl group, or any other group disclosed herein) is substituted with one or more substituents, where valence permits, preferably 1 to 6 substituents, at any available point of attachment. Exemplary substituents include, but are not limited to, one or more of the following groups: hydrogen, halogen (e.g., a single halogen substituent or multiple halo substituents forming, in the latter case, groups such as CF₃ or an alkyl group bearing CCl₃), cyano, nitro, oxo (i.e., ═O), CF₃, OCF₃, alkyl, halogen-substituted alkyl, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, where each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle, and aryl can themselves be optionally substituted. The term “optionally substituted” refers to the embodiments in which a molecule, molecular moiety or substituent group (e.g., alkyl, cycloalkyl, alkenyl, cycloalkenyl, alkynyl, heterocycle, or aryl group, or any other group disclosed herein) may or may not be substituted with aforementioned one or more substituents.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid or phenol, zwitterions (“inner salts”) may be formed and are included within the term “salt(s)” as used herein. Pharmaceutically-acceptable (i.e., non-toxic, physiologically-acceptable) salts are preferred, although other salts are also useful, e.g., in isolation or purification steps which may be employed during preparation. Salts of the compounds of the present invention may be formed, for example, by reacting a compound described herein with an amount of acid or base, such as an equivalent amount, in a medium such as one in which the salt precipitates, or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid; for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such as but not limited to a phenol or carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl) ethylenediamine), N-methyl-D-glucamines, N-methyl-D-glycamides, and t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl and stearyl chlorides, bromides, and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention. As used herein, any depicted structure of the compound includes the tautomeric forms thereof.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives, or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to or greater than 95%, equal to or greater than 99% of the compounds (“substantially pure” compounds), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specification, groups and substituents thereof may be chosen to provide stable moieties and compounds.

Definitions of specific functional groups and chemical terms are described in more detail herein. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito (1999)

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

The present invention also includes isotopically labeled compounds, which are identical to the compounds disclosed herein, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine, and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, or an enantiomer, diastereomer, tautomer, or pharmaceutically-acceptable salt or solvate thereof, which contain the aforementioned isotopes and/or other isotopes of other atoms are within the scope of this invention. Certain isotopically labeled compounds of the present invention, for example, those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example, increased in vivo half-life or reduced dosage requirements, and hence may be preferred in some circumstances. Isotopically-labeled compounds can generally be prepared by carrying out the procedures disclosed in the Schemes and/or in the Examples below, by substituting a readily-available isotopically-labeled reagent for a non-isotopically-labeled reagent.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. For purposes of this invention, heteroatoms such as nitrogen may have hydrogen substituents and/or any permissible substituents of organic compounds described herein which satisfy the valences of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of proliferative disorders. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

As used herein, the terms “cancer” and, equivalently, “tumor” refer to a condition in which abnormally replicating cells of host origin are present in a detectable amount in a subject. The cancer can be a malignant or non-malignant cancer. Cancers or tumors include, but are not limited to, biliary tract cancer; brain cancer; breast cancer; cervical cancer; choriocarcinoma; colon cancer; endometrial cancer; esophageal cancer; gastric (stomach) cancer; intraepithelial neoplasms; leukemias; lymphomas; liver cancer; lung cancer (e.g., small cell and non-small cell); melanoma; neuroblastomas; oral cancer; ovarian cancer; pancreatic cancer; prostate cancer; rectal cancer; renal (kidney) cancer; sarcomas; skin cancer; testicular cancer; and thyroid cancer; as well as other carcinomas and sarcomas. Cancers can be primary or metastatic. Diseases other than cancers may be associated with mutational alternation of component of Ras signaling pathways and the compound disclosed herein may be used to treat these non-cancer diseases. Such non-cancer diseases may include: neurofibromatosis; Leopard syndrome; Noonan syndrome; Legius syndrome; Costello syndrome; cardio-facio-cutaneous syndrome; hereditary gingival fibromatosis type 1; autoimmune lymphoproliferative syndrome; and capillary malformation-arterovenous malformation.

As used herein, “effective amount” refers to any amount that is necessary or sufficient for achieving or promoting a desired outcome. In some instances, an effective amount is a therapeutically effective amount. A therapeutically effective amount is any amount that is necessary or sufficient for promoting or achieving a desired biological response in a subject. The effective amount for any particular application can vary depending on such factors as the disease or condition being treated, the particular agent being administered, the size of the subject, or the severity of the disease or condition. One of ordinary skill in the art can empirically determine the effective amount of a particular agent without necessitating undue experimentation.

As used herein, the term “subject” refers to a vertebrate animal. In one embodiment, the subject is a mammal or a mammalian species. In one embodiment, the subject is a human. In other embodiments, the subject is a non-human vertebrate animal, including, without limitation, non-human primates, laboratory animals, livestock, racehorses, domesticated animals, and non-domesticated animals.

### Compounds

Novel compounds as Kv1.3 potassium channel blockers are described. Applicants have surprisingly discovered that the compounds disclosed herein exhibit potent Kv1.3 potassium channel-inhibiting properties. Additionally, Applicants have surprisingly discovered that the compounds disclosed herein selectively block the Kv1.3 potassium channel and do not block the hERG channel and thus have desirable cardiovascular safety profiles.

In one aspect, a compound of Formula I or a pharmaceutically-acceptable salt thereof is described,

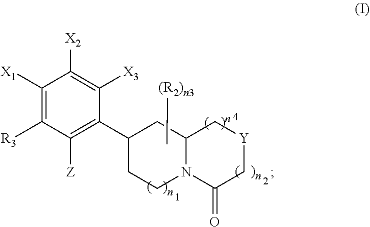

where

Y is C(R₂)₂, NR₁, or O;

Z is ORₐ;

X₁ is H, halogen, or alkyl;

X₂ is H, halogen, CN, alkyl, cycloalkyl, halogenated cycloalkyl, or halogenated alkyl;

X₃ is H, halogen, halogenated alkyl, or alkyl;

or alternatively X₁ and X₂ and the carbon atoms they are connected to taken together form an optionally substituted 5- or 6-membered aryl;

or alternatively X₂ and X₃ and the carbon atoms they are connected to taken together form an optionally substituted 5- or 6-membered aryl;

each occurrence of R₁ is independently H, alkyl, alkenyl, cycloalkyl, heteroalkyl, cycloheteroalkyl, aryl, heteroaryl, (CR₆R₇)ₙ₆ORₐ, (CR₆R₇)ₙ₆N(Rₐ)₂, (C═O)Rₐ, (C═O)ORₐ, (CR₆R₇)ₙ₆(C═O)NRₐR_{b}, SO₂Rₐ, or (CR₆R₇)ₙ₆-heterocycle;

each occurrence of R₂ is independently H, halogen, CN, alkyl, cycloalkyl, heteroalkyl, cycloheteroalkyl, (CR₆R₇)ₙ₆ORₐ, (CR₆R₇)ₙ₆-heterocycle, (C═O)Rₐ, (C═O)ORₐ, (CR₆R₇)ₙ₆NRₐ(C═O)Rₐ, (CR₆R₇)ₙ₆N(Rₐ)₂, NRₐ(CR₆R₇)ₙ₆ORₐ, (C═O)NRₐ(CR₆R₇)ₙ₆ORₐ, (C═O)Rₐ, (CR₆R₇)ₙ₆(C═O)NRₐR_{b}, aryl, or heteroaryl, where each R₂ may be attached to any one of the carbon ring atoms of

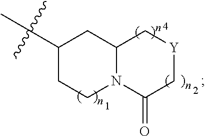

R₃ is H, alkyl, or halogen;

each occurrence of R₆ and R₇ are independently H, alkyl, cycloalkyl, optionally substituted aryl, or optionally substituted heteroaryl;

each occurrence of Rₐ and R_{b} are independently H, alkyl, alkenyl, cycloalkyl, saturated heterocycle, aryl, or heteroaryl; or alternatively Rₐ and R_{b} together with the nitrogen atom that they are connected to form an optionally substituted heterocycle;

the heterocycle includes 1-3 heteroatoms each selected from the group consisting of N, O, and S;

the alkyl, cycloalkyl, heteroalkyl, cycloheteroalkyl, heterocycle, aryl, and heteroaryl, in X₁, X₂, X₃, R₁, R₂, R₃, R₆, R₇, Rₐ, and R_{b}, where applicable, are each independently and optionally substituted by 1-4 substituents each independently selected from the group consisting of alkyl, cycloalkyl, halogenated alkyl, halogenated cycloalkyl, halogen, CN, R₈, OR₈, —(CH₂)₁₋₂OR₈, N(R₈)₂, (C═O)R₈, (C═O)N(R₈)₂, NR₈(C═O)R₈, and oxo where valence permits;

each occurrence of R₈ is independently H, alkyl, cycloalkyl, or a heterocycle optionally substituted by alkyl; or alternatively the two R₈ groups together with the nitrogen atom that they are connected to form a heterocycle optionally substituted by alkyl and including the nitrogen atom and 0-3 additional heteroatoms each selected from the group consisting of N, O, and S; n₁ is an integer from 0-1;

n₂ is an integer from 0-2;

n₃ is an integer from 0-3;

n₄ is an integer from 1-2; and

n₆ is an integer from 0-3.

In some embodiments, the structural moiety

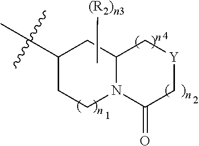

has the structure of

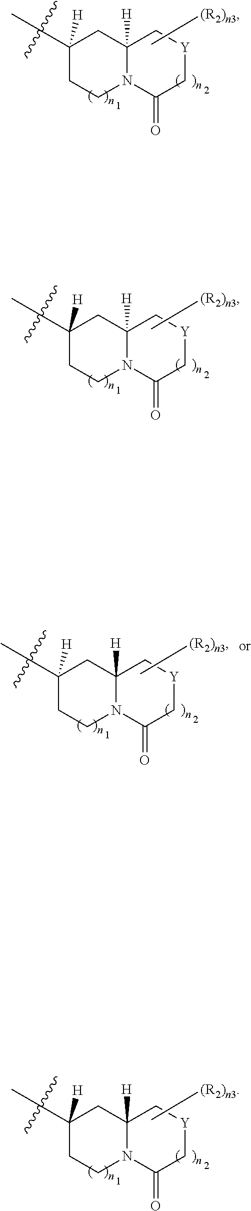

In some specific embodiments, the structural moiety

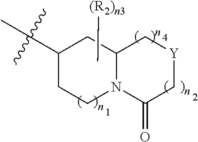

has the structure of

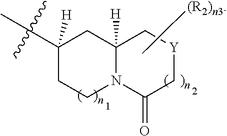

In some embodiments, the structural moiety

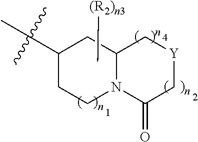

has the structure of

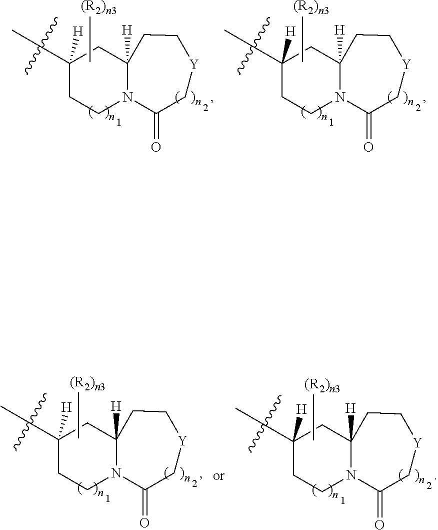

In some specific embodiments, the structural moiety

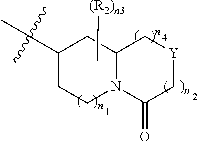

has the structure of

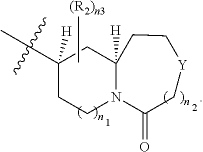

In some embodiments, n₁ is 1. In some embodiments, n₁ is 0. In some embodiments, n₂ is an integer from 0-2. In some embodiments, n₂ is an integer from 1-2. In some embodiments, n₂ is 0. In some embodiments, n₂ is 1 or 2. In some embodiments, n₂ is 1.

In some embodiments, the structural moiety

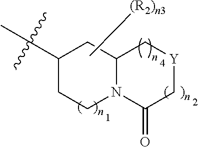

has the structure of

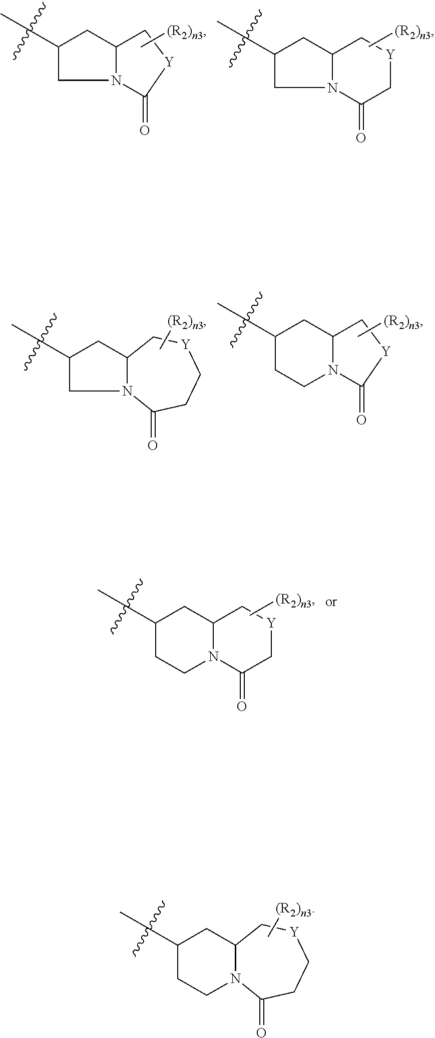

In some embodiments, the structural moiety

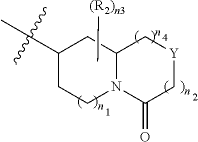

has the structure of

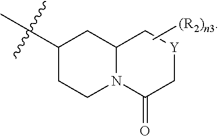

In some embodiments, the structural moiety

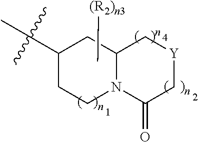

has the structure of

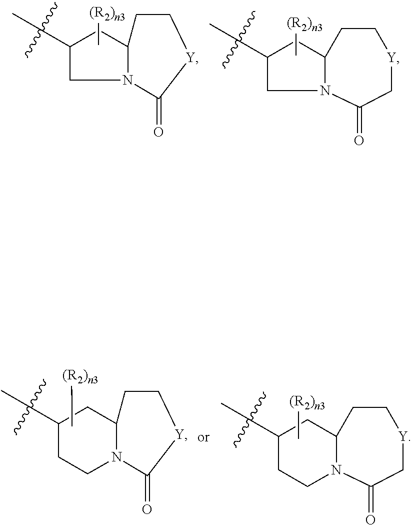

In some embodiments, the structural moiety

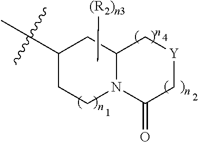

has the structure of

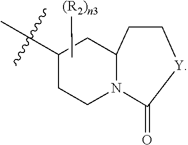

In some embodiments, Y is C(R₂)₂. In other embodiments, Y is NR₁. In still other embodiments, Y is O.

In some embodiments, the structural moiety

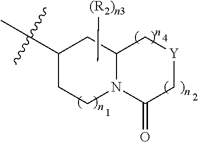

has the structure of

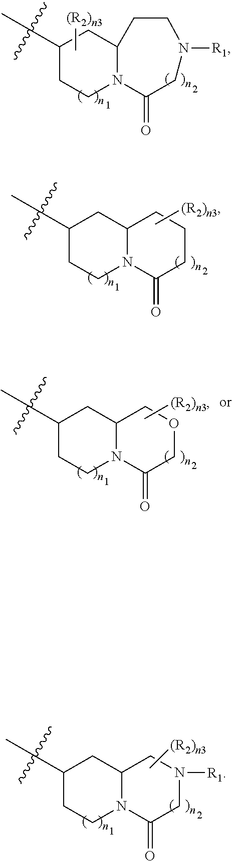

In some specific embodiments, the structural moiety

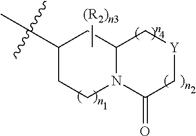

has the structure of

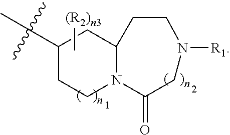

In some specific embodiments, the structural moiety

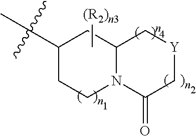

has the structure of

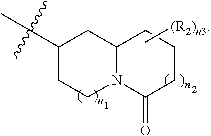

In some specific embodiments, the structural moiety

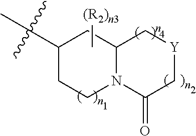

has the structure of

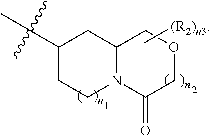

In some specific embodiments, the structural moiety

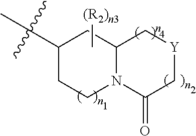

has the structure of

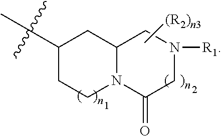

In some specific embodiments, the structural moiety

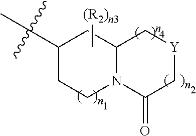

has the structure of

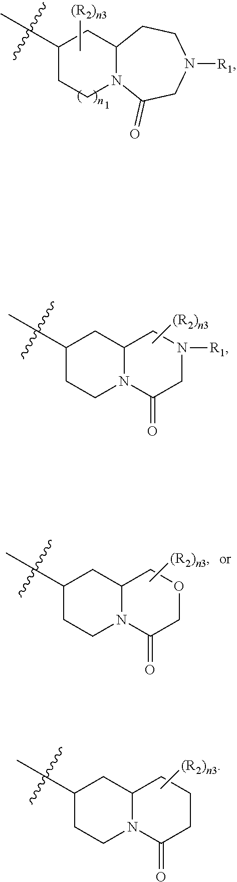

In some specific embodiments, the structural moiety

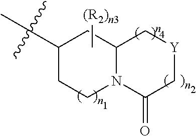

has the structure of

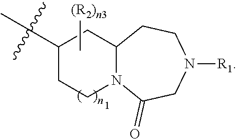

In some embodiments, the structural moiety

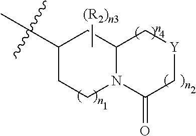

has the structure of

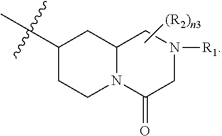

In some embodiments, the structural moiety

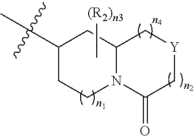

has the structure of

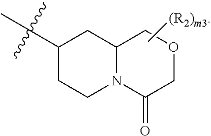

In some embodiments, the structural moiety

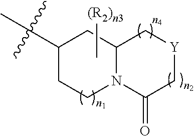

has the structure of

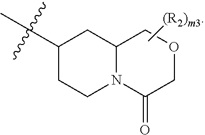

In some embodiments, R₁ is H, alkyl, alkenyl, cycloalkyl, heteroalkyl, or cycloheteroalkyl.

In some embodiments, R₁ is H. In some embodiments, R₁ is alkyl, such as Me, Et, propyl, isopropyl, n-butyl, iso-butyl, or sec-butyl. In other embodiments, R₁ is cycloalkyl, such as cyclopropyl, cyclobutyl, cyclopentyl, or cyclohexyl.

In some embodiments, R₁ is heteroalkyl. In some specific embodiments, R₁ is alkyl ethers, secondary and tertiary alkyl amines, or alkyl sulfides, such as —CH₂—CH₂—OMe, —CH₂—CH₂—OEt, —CH₂—CH₂—OPr, —CH₂—CH₂—SMe, —CH₂—CH₂—SEt, —CH₂—CH₂—SPr, —CH₂—CH₂—NHIMe, —CH₂—CH₂—NMe₂, —CH₂—CH₂—NEtMe, or —CH₂—CH₂—NEt₂. In some embodiments, R₁ is cycloheteroalkyl. Non-limiting examples of cycloheteroalkyl include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane.

In some embodiments, R₁ is aryl or heteroaryl. In some embodiments, R₁ is (C═O)Rₐ, (C═O)ORₐ, (C═O)NRₐR_{b}, SO₂Rₐ, (CR₆R₇)ₙ₆ORₐ, or (CR₆R₇)ₙ₆N(Rₐ)₂. In some embodiments, R₁ is (CR₆R₇)ₙ₆(C═O)NRₐR_{b}, SO₂Rₐ or (CR₆R₇)ₙ₆-heterocycle. In some specific embodiments, R₁ is (C═O)Rₐ. In some specific embodiments, Rₐ and R_{b} are each independently H, alkyl, or an alkyl substituted by one or more OR₈. In some specific embodiments, R₈ is H or alkyl.

In some embodiments, R₁ is selected from the group consisting of H, —CH₃, —(CH₂)₂OH, —(CH₂)₂NH₂, —CONH₂, —CONHMe, —CONMe₂, —CONEt₂, SO₂Me, or SO₂Et. In other embodiments, R₁ is selected from the group consisting of H,

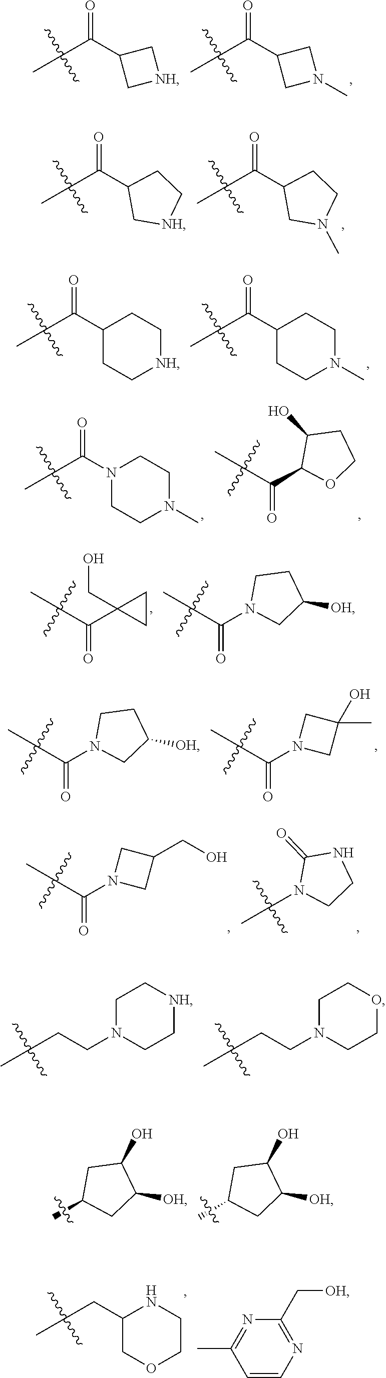

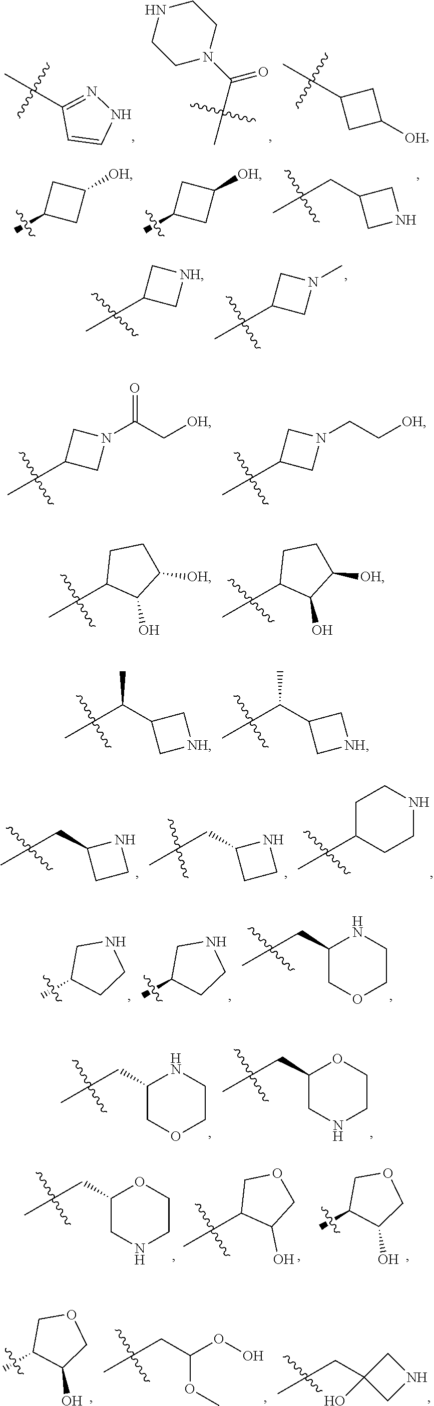

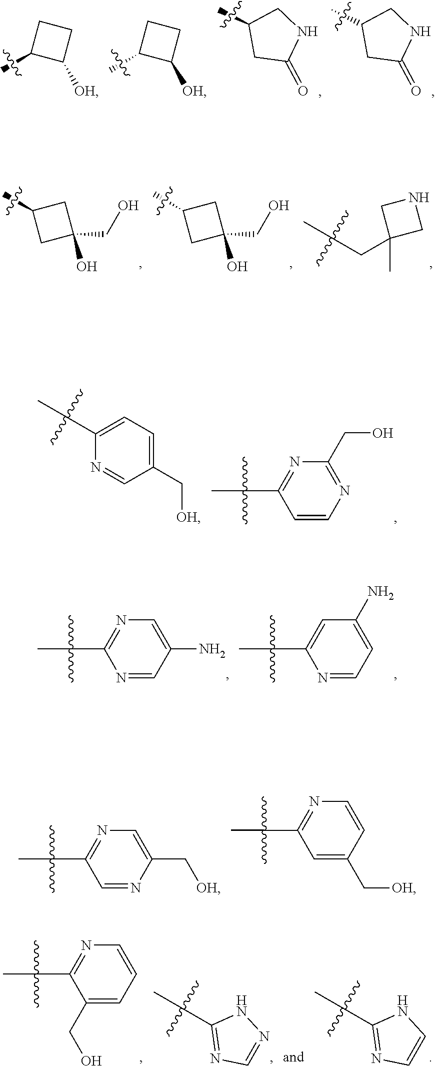

In still other embodiments, R₁ is selected from the group consisting of

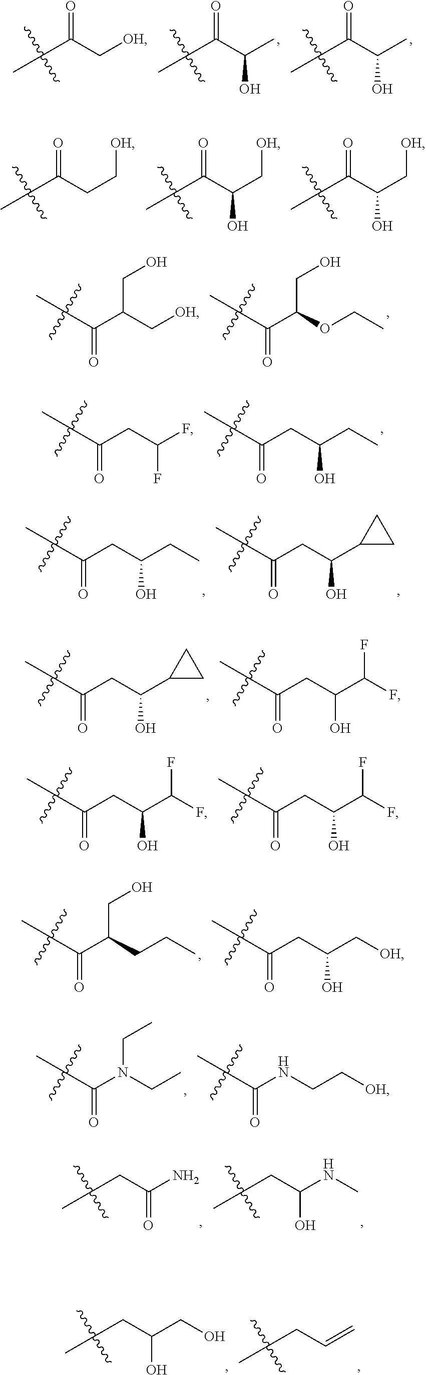

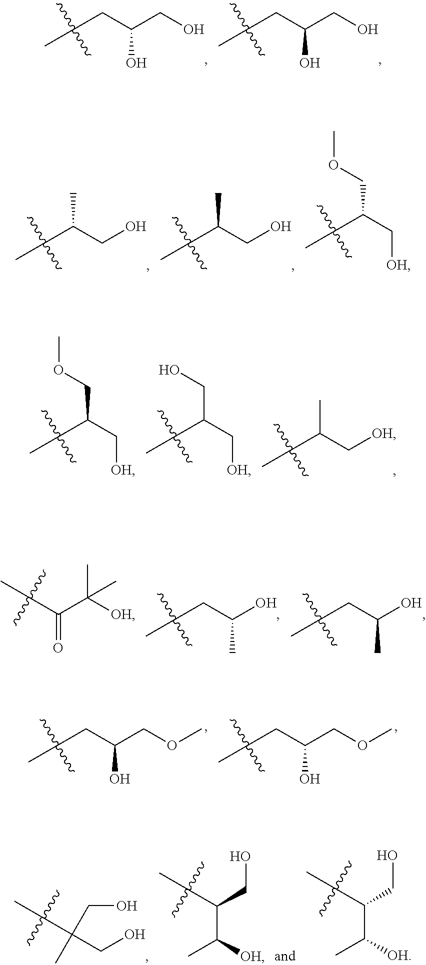

In some embodiments, at least one occurrence of R₂ is H, CN, alkyl, heteroalkyl, cycloalkyl, or cycloheteroalkyl. In some embodiments, at least one occurrence of R₂ is H. In some embodiments, at least one occurrence of R₂ is alkyl, such as Me, Et, propyl, isopropyl, n-butyl, iso-butyl, or sec-butyl. In other embodiments, at least one occurrence of R₂ is cycloalkyl, such as cyclopropyl, cyclobutyl, cyclopentyl, or cyclohexyl. In some embodiments, at least one occurrence of R₂ is aryl or heteroaryl.

In some embodiments, at least one occurrence of R₂ is (CR₆R₇)ₙ₆ORₐ, (CR₆R₇)ₙ₆-heterocycle, (C═O)Rₐ, (C═O)ORₐ, (CR₆R₇)ₙ₆NRₐ(C═O)Rₐ, (CR₆R₇)ₙ₆N(Rₐ)₂, NRₐ(CR₆R₇)ₙ₆ORₐ, (C═O)NRₐ(CR₆R₇)ₙ₆ORₐ, or (CR₆R₇)ₙ₆(C═O)NRₐR_{b}. In some embodiments, each R₂ may be attached to any one of the carbon ring atoms of

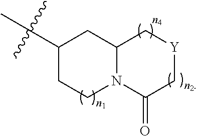

In some specific embodiments, at least one occurrence of R₂ is —CH₃, —CH₂—OH, —CH₂—CH₂—OH, —CH(OH)—CH₃, —CH₂—NH₂,

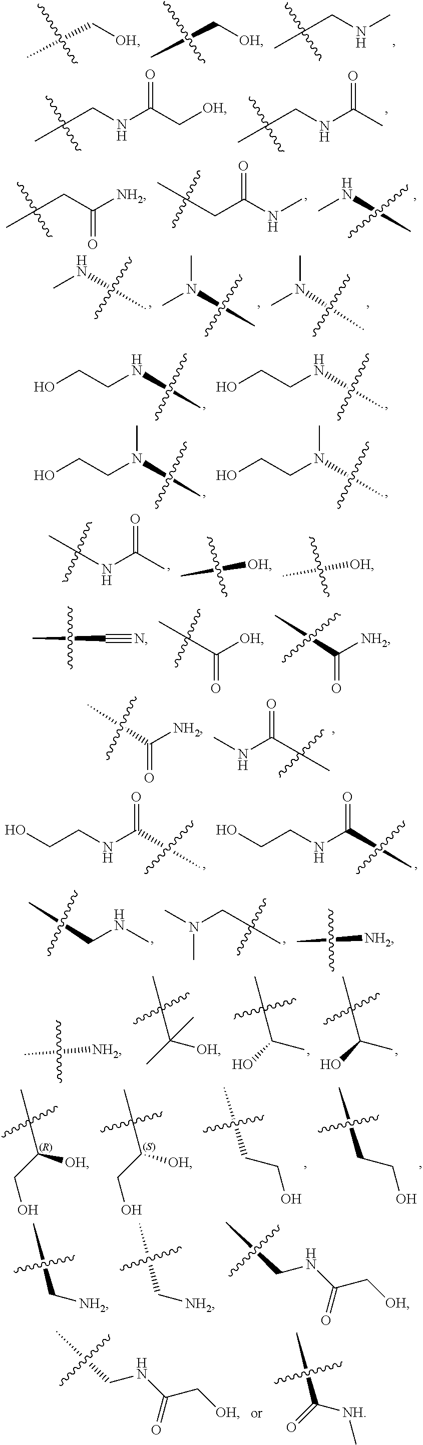

In some embodiments, at least one occurrence of R₂ is ORₐ. In some embodiments, at least one occurrence of R₂ is N(R₁)₂. In some embodiments, at least one occurrence of R₂ is (C═O)Rₐ. In some embodiments, at least one occurrence of R₂ is (C═O)NRₐR_{b}. In some embodiments, at least one occurrence of R₂ is aryl. In some embodiments, R₂ is heteroaryl. In some embodiments, at least one occurrence of R₂ is heteroalkyl or cycloheteroalkyl. In some embodiments, R₂ is heteroalkyl. In some specific embodiments, R₂ is alkyl ethers, secondary and tertiary alkyl amines, or alkyl sulfides, such as —CH₂—CH₂—OMe, —CH₂—CH₂—OEt, —CH₂—CH₂—OPr, —CH₂—CH₂—SMe, —CH₂—CH₂—SEt, —CH₂—CH₂—SPr, —CH₂—CH₂—NHMe, —CH₂—CH₂—NMe₂, —CH₂—CH₂—NEtMe, or —CH₂—CH₂—NEt₂. In some embodiments, R₂ is cycloheteroalkyl. Non-limiting examples of cycloheteroalkyl include pyrrolidyl, tetrahydrofuryl, tetrahydrothiofuranyl, piperidyl, piperazyl, tetrahydropyranyl, morpholino, 1,3-diazepane, 1,4-diazepane, 1,4-oxazepane, and 1,4-oxathiapane. In some embodiments, at least one occurrence of R₂ is

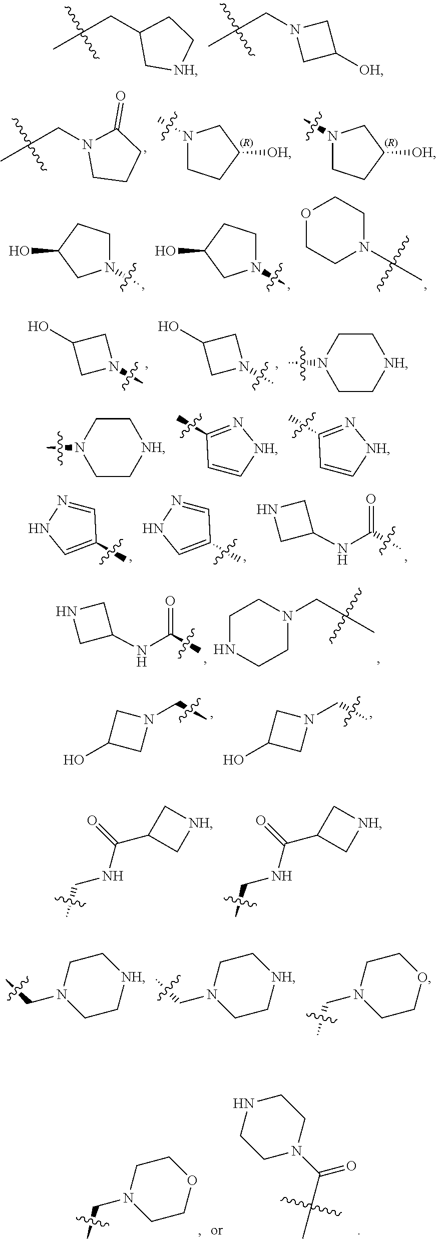

In some embodiments, n₁ is 0. In some embodiments, n₁ is 1. In some embodiments, n₂ is 0. In some embodiments, n₂ is 1. In some embodiments, n₃ is 0, 1, 2, or 3. In some embodiments, n₃ is 0. In some embodiments, n₃ is 1. In some embodiments, n₃ is 2. In some embodiments, n₄ is 1. In some embodiments, n₄ is 2. In some embodiments, n₆ is 0. In some embodiments, n₆ is 1. In some embodiments, n₆ is 2. In some embodiments, n₆ is 3.

In some embodiments, R₈ is H or alkyl. In other embodiments, R₈ is optionally substituted heterocycle. In still other embodiments, the two R₈ groups together with the nitrogen atom that they are connected to form an optionally substituted heterocycle including the nitrogen atom and 0-3 additional heteroatoms each selected from the group consisting of N, O, and S.

In some embodiments, Z is ORₐ. In some embodiments, Z is OH, OMe, OEt, OPr, O-i-Pr, O-t-Bu, O-iso-Bu, O-sec-Bu, or OBu. In some embodiments, Z is OH.

In some embodiments, X₁ is H, halogen, or alkyl. In any one of the embodiments described herein, X₁ may be H or alkyl. In some embodiments, X₁ is Me, Et, Pr, i-Pr, or Bu. In some embodiments, X₁ is H or halogen. In other embodiments, X₁ is alkyl. In some embodiments, X₁ is H, F, Cl, Br, or Me. In some embodiments, X₁ is H, F, or Cl. In some embodiments, X₁ is F or Cl. In some embodiments, X₁ is H or Cl. In some embodiments, X₁ is F. In some embodiments, X₁ is H.

In some embodiments, X₂ is H, halogen, CN, alkyl, halogenated alkyl, cycloalkyl, or halogenated cycloalkyl. In any one of the embodiments described herein, X₂ may be H, halogen, fluorinated alkyl, or alkyl. In some embodiments, X₂ is H or halogen. In other embodiments, X₂ is fluorinated alkyl or alkyl. In other embodiments, X₂ is cycloalkyl. In some embodiments, X₂ is H, F, Cl, Br, Me, CF₂H, CF₂Cl, or CF₃. In some embodiments, X₂ is H, F, or Cl. In some embodiments, X₂ is F or Cl. In some embodiments, X₂ is H or Cl. In some embodiments, X₂ is F. In some embodiments, X₂ is CF₃. In some embodiments, X₂ is CF₂Cl. In some embodiments, X₂ is Cl.

In some embodiments, X₃ is H, halogen, alkyl, or halogenated alkyl. In any one of the embodiments described herein, X₃ may be H, halogen, fluorinated alkyl, or alkyl. In some embodiments, X₃ is H or halogen. In other embodiments, X₃ is fluorinated alkyl or alkyl. In some embodiments, X₃ is H, F, Cl, Br, Me, CF₂H, CF₂Cl, or CF₃. In some embodiments, X₃ is H, F, or Cl. In some embodiments, X₃ is F or Cl. In some embodiments, X₃ is H or Cl. In some embodiments, X₃ is F. In some embodiments, X₃ is CF₃. In some embodiments, X₃ is CF₂Cl. In some embodiments, X₃ is Cl.

In some embodiments, the structural moiety

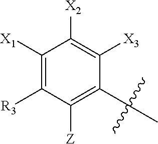

has the structure of

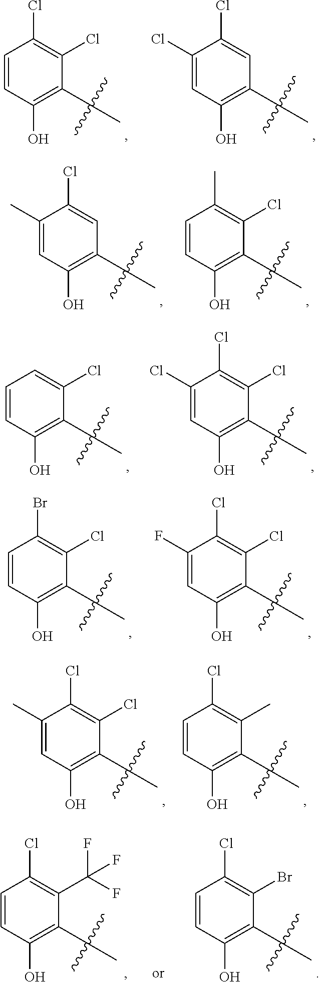

In any one of the embodiments described herein, R₃ is H, alkyl, or halogen. In some embodiments, R₃ is halogen. In some embodiments, R₃ is H, halogen, or alkyl. Non-limiting examples of alkyl include Me, Et, propyl, isopropyl, n-butyl, iso-butyl, t-butyl, and sec-butyl. In some embodiments, R₃ is H.

In some embodiments, the compound of Formula I has a structure of Formula II′ or II,

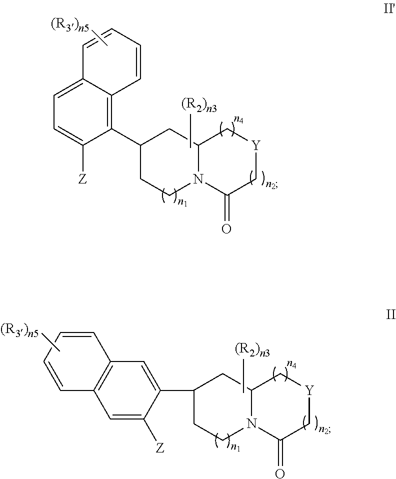

where each occurrence of R_{3′} is independently H, halogen, or alkyl, n₅ is an integer from 0-3 and other substituents are as defined herein.

In some embodiments, Z is ORₐ. In some embodiments, Z is OH, OMe, OEt, OPr, O-i-Pr, O-t-Bu, O-iso-Bu, O-sec-Bu, or OBu. In some embodiments, Z is OH.

In some embodiments, n₅ is an integer from 0-3. In some embodiments, n₅ is an integer from 1-3. In some embodiments, n₅ is 0. In some embodiments, n₅ is 1 or 2. In some embodiments, n₅ is 1. In some embodiments, R_{3′} is H or alkyl. In some embodiments, R_{3′} is H. In some embodiments, R_{3′} is alkyl. In some embodiments, R_{3′} is halogen.

In any one of the embodiments described herein, at least one occurrence of Rₐ or R_{b} is independently H, alkyl, cycloalkyl, saturated heterocycle, aryl, or heteroaryl. In some embodiments, at least one occurrence of Rₐ or R_{b} is independently H, Me, Et, Pr, or Bu. In some embodiments, at least one occurrence of Rₐ or R_{b} is independently a heterocycle selected from the group consisting of

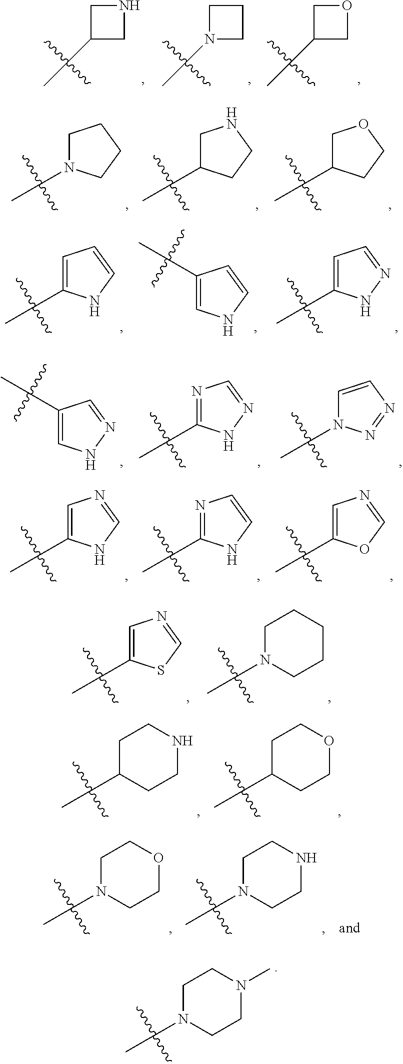

and the heterocycle is optionally substituted by alkyl, OH, oxo, or (C═O)C₁₋₄alkyl where valence permits.

In some embodiments, Rₐ and R_{b} together with the nitrogen atom that they are connected to form an optionally substituted heterocycle including the nitrogen atom and 0-3 additional heteroatoms each selected from the group consisting of N, O, and S.

In some embodiments, the heterocycle is selected from the group consisting of

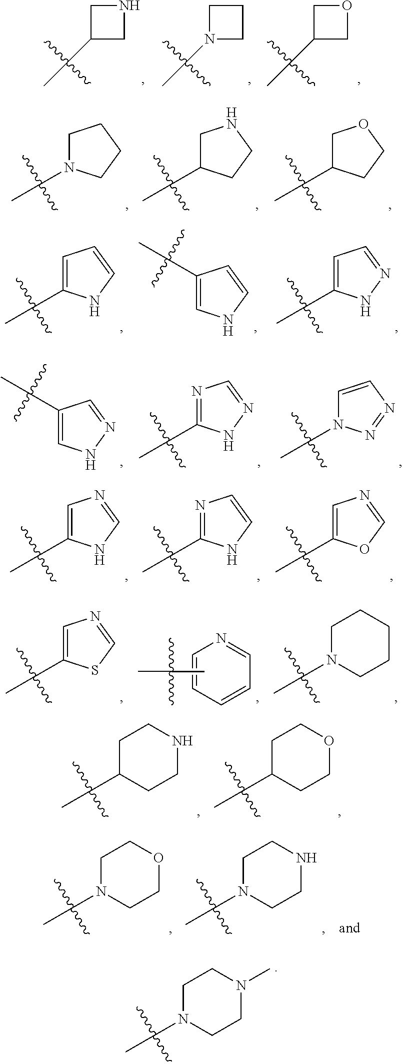

In some embodiments, the compound of Formula I is selected from the group consisting of compounds 1-62 as shown in Table 4 below.

In some embodiments, the compound of Formula I is selected from the group consisting of compounds 63-78, 83-85, 87-88, 90-94, 96-97, 99-104, 109-176, 180-208, 213-220, 223-293 as shown in Table 5 below.

### Abbreviations

ACN Acetonitrile
Alloc Allyoxycarbonyl
Boc or boc tert-Butyloxycarbonyl
DCM Dichloromethane
DIEA N,N-Diisopropylethylamine
DIPA Diisopropylamine
DMF 4-Dimethylaminopyridine
EA Ethyl acetate
EDCI or EDC 1-Ethyl-3-(3-dimethylaminopropyl)carbodiimide
Fmoc 9-Fluorenylmethyl carbamate
HATU N-[(dimethylamino)(3H-1,2,3-triazolo(4,4-b)pyridin-3-yloxy)methylene]-N-methylmethaneaminium hexafluorophosphate
LiHMDS Lithium hexamethyldisilazide
HOBT 1-Hydroxybenzotriazole
PE Petroleum ether
TBTU 2-(1H-Benzotriazole-1-yl)-1,1,3,3-tetramethylaminium tetrafluoroborate
TEA Triethylamine
TFA Trifluoroacetic acid
THF Tetrahydrofuran
Troc 2,2,2-Trichloroethoxycarbonyl
TsOH p-Toluenesulfonic acid
   Methods ofP reparation

Following are general synthetic schemes for manufacturing compounds of the present invention. These schemes are illustrative and are not meant to limit the possible techniques one skilled in the art may use to manufacture the compounds disclosed herein. Different methods will be evident to those skilled in the art. Additionally, the various steps in the synthesis may be performed in an alternate sequence or order to give the desired compound(s). All documents cited herein are incorporated herein by reference in their entirety. For example, the following reactions are illustrations, but not limitations of the preparation of some of the starting materials and compounds disclosed herein.

Schemes 1-8 below describe synthetic routes which may be used for the synthesis of compounds of the present invention, e.g., compounds having a structure of Formula I or a precursor thereof. Various modifications to these methods may be envisioned by those skilled in the art to achieve similar results to that of the inventions given below. In the embodiments below, the synthetic route is described using compounds having the structure of Formula I or a precursor thereof as examples. The general synthetic routes described in Schemes 1-8 and examples described in the Example section below illustrate methods used for the preparation of the compounds described herein.

Compounds I-1a and I-2 as shown immediately below in Scheme 1 can be prepared by any method known in the art and/or are commercially available. As shown in Scheme 1, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH and amine group. Other substituents are defined herein. As shown in Scheme 1, the core of the compounds of Formula I can be synthesized from a suitable substituted bromo or iodo benzene I-1a that is converted to the corresponding boronic acid I-1b by metalation with for example n-butyl lithium and reaction with a trialkyl borate such as trimethyl borate. Ketoester I-2 is reacted with a base such as LiHMDS and N-phenyl triflimide to form the enol trifluoromethanesulfonate I-3. Coupling of enol trifluoromethanesulfonate I-3 with boronic acid I-1b in the presence of a catalyst such as 1,1′-bis(diphenylphosphino)-ferrocene dichloropalladium(II) (Pd(dppf)Cl₂) gives cyclic amine I-4. Hydrogenation of I-4 over a catalyst such as platinum oxide yields the saturated cyclic amine ester I-5a. Selective removal of the protecting group on nitrogen of compound I-5a provides the corresponding cyclic amine ester I-5c. The protecting group in compound I-5c can then be removed, and the resulting compound with the free phenol OH group can optionally be converted to a compound of Formula I using methods known in the art.

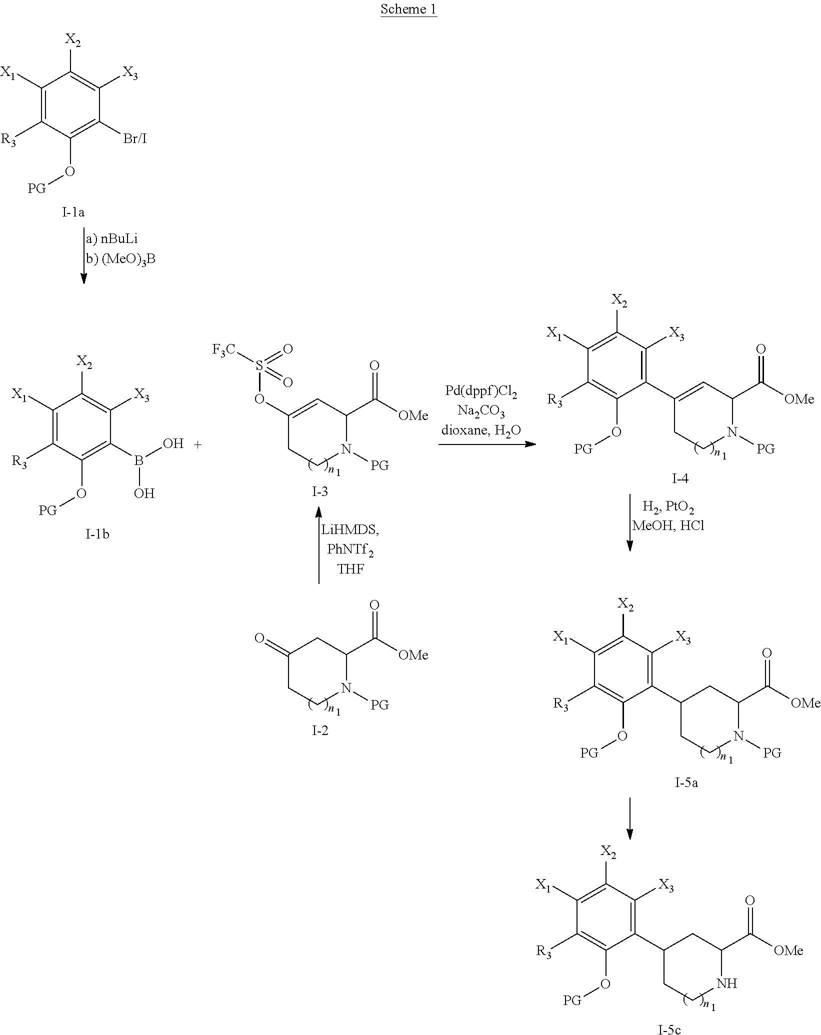

Compound I-1a as shown immediately below in Scheme 2 can be prepared by any method known in the art and/or is commercially available. As shown in Scheme 2, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. As shown in Scheme 2, compounds of Formula I where n₁=1 can be prepared by an alternative route shown therein. The iodo or bromo benzene I-1a is coupled with a pyridine boronate ester I-6 in the presence of a palladium catalyst such as Pd(dppf)Cl₂ to form the 4-aryl pyridine ester I-8 or with cyanopyridine boronate ester I-7 to form the 4-aryl pyridine nitrile I-9. Hydrogenation of ester I-8 over a catalyst such as platinum oxide provides the 4-aryl piperidine I-5b. The protecting group in compound I-5b can then be removed, and the resulting compound with the free phenol OH group can optionally be converted to a compound of Formula I using methods known in the art.

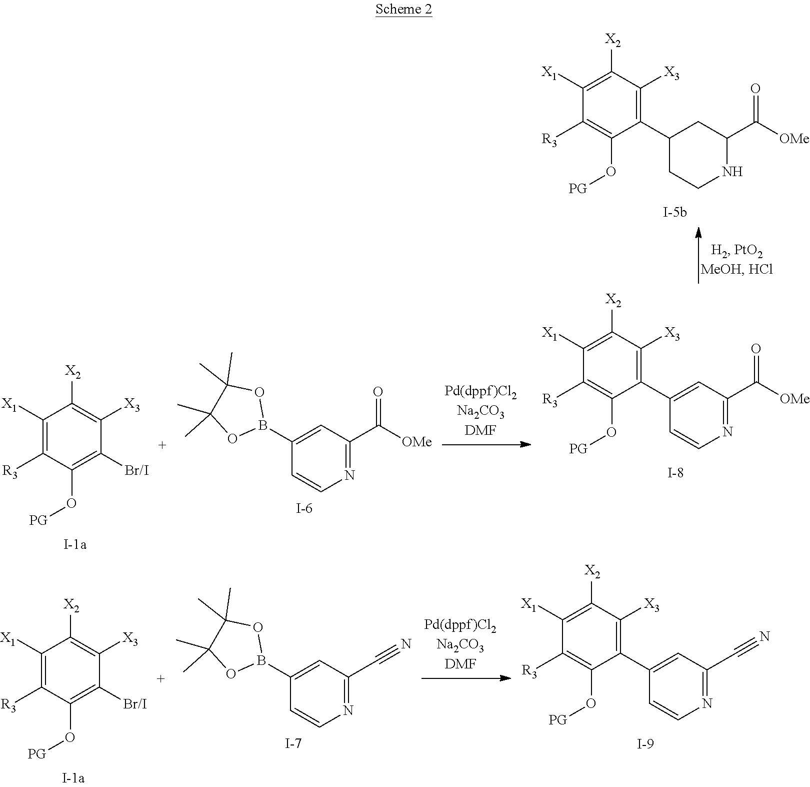

As shown in Scheme 3, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. The intermediates, aminomethyl heterocycle I-12a and I-12b can be obtained by several routes shown immediately below in Scheme 3. For compounds of Formula I where n₁=1, the pyridine nitrile I-9 (as shown in Scheme 2) can be converted to the primary amide I-10 by hydrolysis with alkaline peroxide, or reduced to the aminomethyl pyridine I-11 with borane-tetrahydrofuran complex. Hydrogenation of the pyridine ring of I-10 or I-11 over a catalyst such as platinum oxide in the presence of an acid such as hydrochloric acid or acetic acid yields the corresponding piperidines I-13 or I-12a respectively. Alternatively, hydrogenation of I-9 under similar conditions gives I-12a directly. In an approach applicable to all ring sizes, ester I-5c (as shown in Scheme 1) is converted to the primary amide I-13 by heating with ammonia in methanol in a sealed vessel. Reduction of amide I-13 with borane-methyl sulfide provides the diamine I-12b. The protecting group in compounds I-12a, I-12b, and I-13 can optionally be removed to afford a compound of Formula I using methods known in the art.

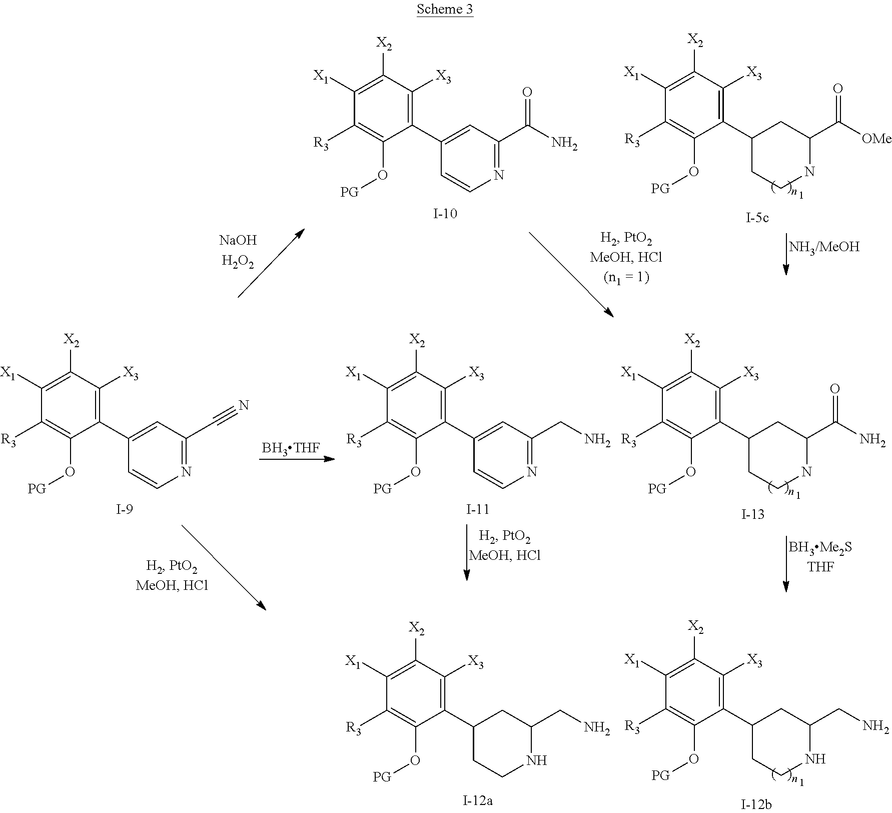

As shown in Scheme 4, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. Diamine I-12b can be used to prepare the bicyclic amide I-17 by one of the two routes shown immediately below in Scheme 4. Acylation of I-12b with a carboxylic acid RaCO₂H using a peptide coupling reagent such as EDCI/HOBT, TBTU, or HATU occurs selectively on the primary amine to give I-14. Acylation of I-14 on the cyclic amine with chloroacetyl chloride gives the chloroacetamide I-15 that cyclizes on treatment with a base such as cesium carbonate in a polar solvent such as DMF to form piperazinone I-16. Deprotection of protecting group on the phenol, for example with boron tribromide if the protecting group is methyl, provides I-17. Alternatively, the primary amine of I-12b is selectively protected (e.g., with a Boc group) to give I-18. An analogous sequence of acylation with chloroacetyl chloride to I-19 and cyclization with base followed by simultaneous deprotection of both the amine and the phenol yields I-20 that can be acylated with a carboxylic acid RaCO₂H under standard conditions to provide I-17. Compound I-20 can be used to provide compounds with other R₄ groups on nitrogen by standard methods.

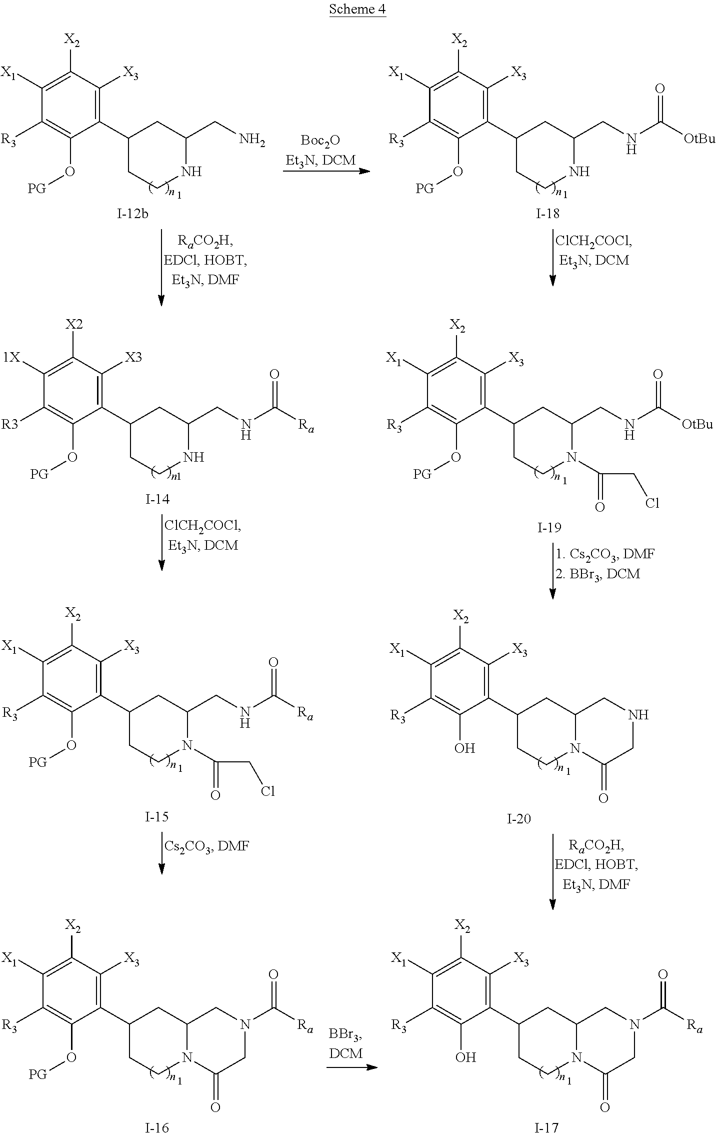

As shown in Scheme 5, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. Compounds of Formula I where Y is oxygen are synthesized as shown immediately below in Scheme 5. The cyclic amine ester I-5c (as shown in Scheme 1) is reduced to alcohol I-21 with for example borane-tetrahydrofuran with heating. I-21 is coupled with the potassium salt of 2-oxirane carboxylic acid using a reagent such as HATU, TBTU, or EDC/HOBt to form epoxy amide I-22. Treatment of epoxide I-22 with a base such as sodium hydride in an inert solvent such as THE results in cyclization to I-23. The hydroxymethyl group in I-23 can be converted to other substituents by standard methods. Removal of the protecting group provides the free phenol.

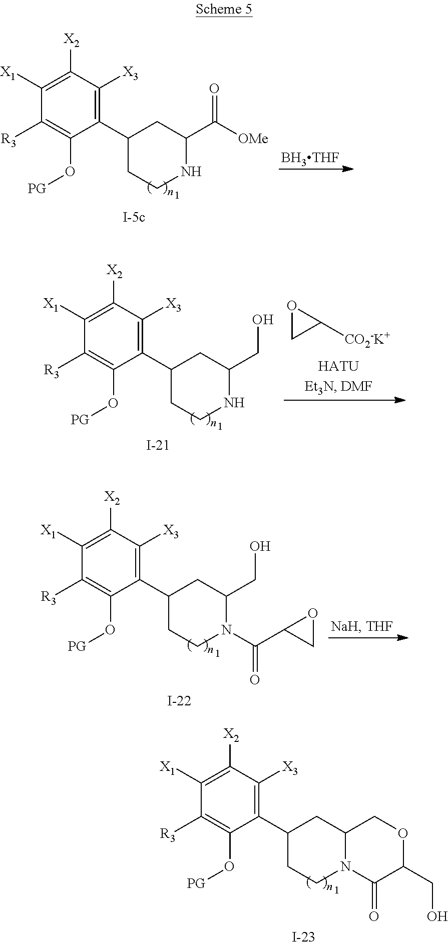

As shown in Scheme 6, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. The compounds of Formula I having a ring system where Y is N (e.g., 8-phenyl-octahydro-4H-pyrido[1,2-a]pyrazine-4-one substituted with R₂ at C₃ position (I-27)) is obtained from the amino alcohol I-21 by either of the two routes shown immediately below in Scheme 6. Acylation of I-21 (as shown in Scheme 5) with a suitably protected amino acid using a coupling reagent such as HATU, TBTU, or EDC/HOBT gives amide I-24. Typically, the amino group is protected with Boc, but other protecting groups such as alloc, troc or Fmoc could also be used. Oxidation of the primary alcohol to the aldehyde I-25 is carried out using the Dess-Martin reagent or Swern oxidation conditions. Removal of the amine protecting group with TFA results in cyclization to I-26 and the imine double bond is then reduced by hydrogenation or with sodium borohydride. In an alternative procedure, the amine of I-21 is first protected with a Boc group to form I-28 which is oxidized to the aldehyde I-29 with Dess-Martin reagent. The aldehyde I-29 undergoes reductive amination with an amino acid ester in the presence of a reducing agent such as sodium triacetoxy borohydride or sodium cyanoborohydride to yield I-30. Removal of the Boc protecting group on the nitrogen followed by heating with a base such as triethylamine in a solvent such as ethanol results in cyclization to I-27. I-27 can be further modified by derivatization of the amine by standard methods and removal of the protecting group to give the free phenol to afform additional compounds of Formula I.

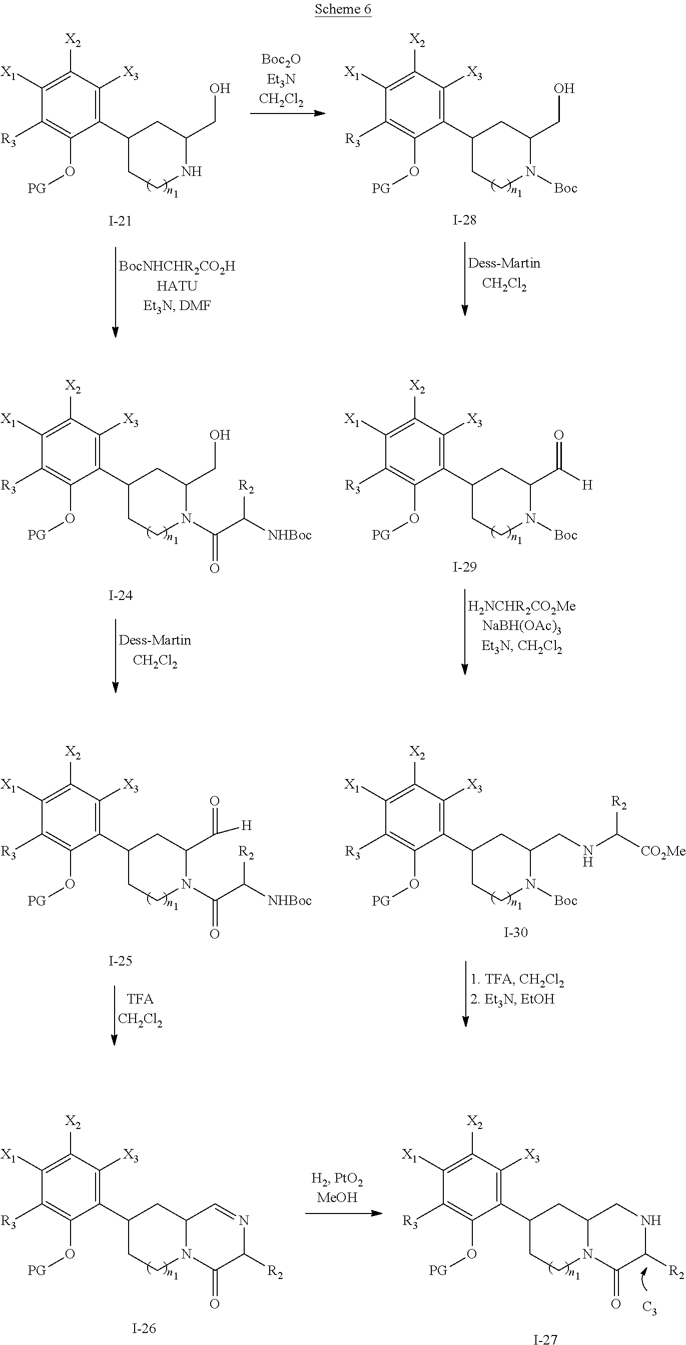

As shown in Scheme 7, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH and amine groups. Other substituents are defined herein. The compounds of Formula I having a ring system where Y is N (e.g., 8-phenyl-octahydro-4H-pyrido[1,2-a]pyrazine-4-one substituted with R₂ at Ci (I-35)) is prepared from the protected amino ester I-5a (as shown in Scheme 1) by the route shown immediately below in Scheme 7. The ester I-5a is first hydrolyzed to the carboxylic acid and converted to the Weinreb amide I-31 by treatment with N,O-dimethyl hydroxylamine and a coupling reagent such as carbonyl diimidazole or EDC/HOBT. Reaction of I-31 with a Grignard reagent, R₂MgBr, forms ketone I-32. The protecting group on nitrogen is then selectively removed. When PG is Boc, the removal of the Boc group can be accomplished by using TFA. The cyclic amine is acylated with a protected amino acid such Bocglycine to give amide I-33. Removal of the Boc group with TFA and concomitant cyclization of the amine onto the ketone forms cyclic imine I-34. Reduction of the imine with sodium borohydride yields cyclic amine I-35 that can be further modified on the amine nitrogen by for example acylation or alkylation by standard methods. Removal of the protecting group on the phenol to give the free phenol may be carried out either before or after derivatization of the amine.

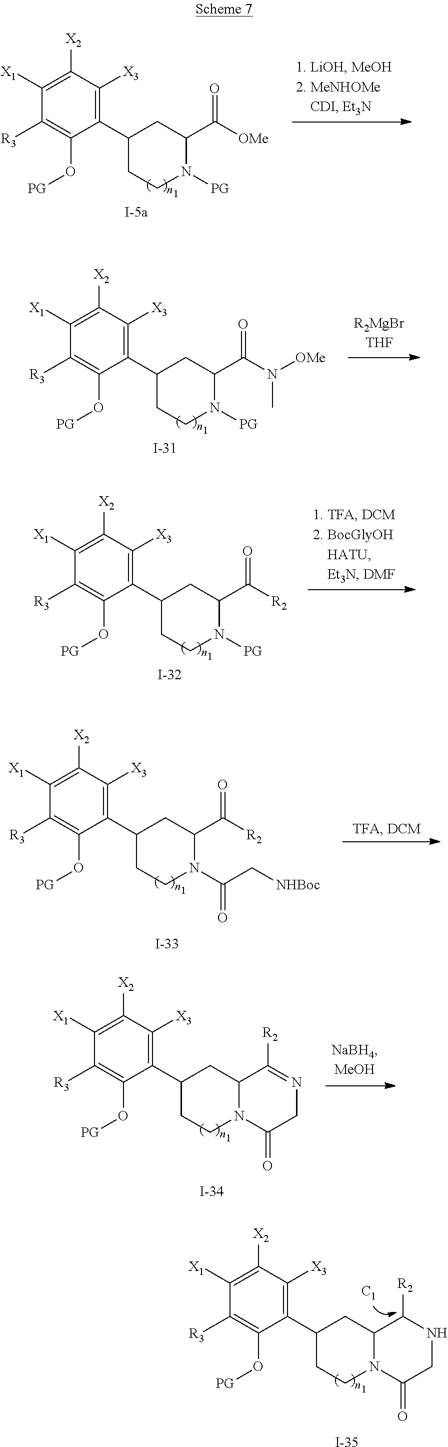

As shown in Scheme 8, PG refers to a protecting group. Non-limiting examples of the protecting groups include Me, allyl, Ac, Boc, other alkoxycarbonyl group, dialkylaminocarbonyl, or another protecting group known in the art suitable for use as protecting groups for OH. Other substituents are defined herein. A stereoselective synthesis of intermediate I-5d is shown immediately below in Scheme 8. The enantiomerically pure piperidone I-36 is synthesized from protected L-aspartic acid and Meldrum's acid by the method described in Org. Syn., 2008, 85, 147, and was then converted to the enol triflate I-37 by treatment with trifluoromethansulfonic anhydride and base according to the procedure described in Syn. Lett. 2009, 71-74. The enol triflate I-37 is coupled with boronic acid I-1b using a palladium catalyst such as Pd(dppf)Cl₂ to yield I-39. Hydrogenation of I-17 over a catalyst such as platinum oxide gives piperidinone I-40 predominantly as the 2S,4S enantiomer, and reduction of the amide using borane methyl sulfide complex provides enantiomerically pure I-5d that can be used in the syntheses outlined in Schemes 3, 4, 5, 6 and 7.

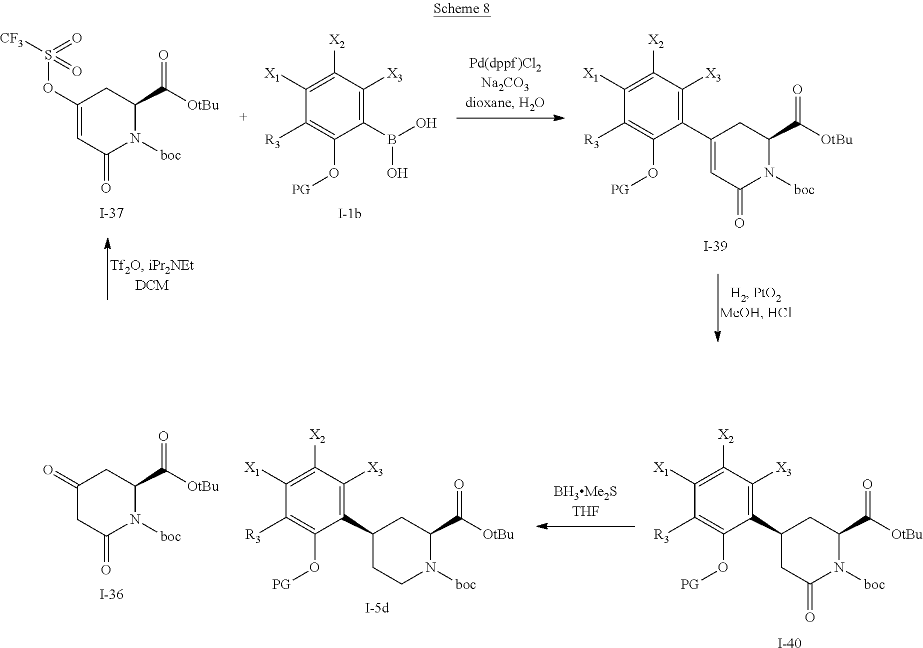

The reactions described in Schemes 1-8 above can be carried out in a suitable solvent. Suitable solvents include, but are not limited to, ACN, methanol, ethanol, DCM, DMF, THF, MTBE, or toluene. The reactions described in Schemes 1-8 may be conducted under inert atmosphere, e.g., under nitrogen or argon, or the reaction may be carried out in a sealed tube. The reaction mixture may be heated in a microwave or heated to an elevated temperature. Suitable elevated temperatures include, but are not limited to, 40, 50, 60, 80, 90, 100, 110, 120° C., or higher, or the refluxing/boiling temperature of the solvent used. The reaction mixture may alternatively be cooled in a cold bath at a temperature lower than room temperature, e.g., 0, −10, −20, −30, −40, −50, −78, or −90° C. The reaction may be worked up by removing the solvent or partitioning of the organic solvent phase with one or more aqueous phases, each optionally containing NaCl, NaHCO₃, or NH₄Cl. The solvent in the organic phase can be removed by vacuum evaporation and the resulting residue may be purified using a silica gel column or HPLC.

### Pharmaceutical Compositions

This invention also provides a pharmaceutical composition including at least one of the compounds as described herein or a pharmaceutically-acceptable salt or solvate thereof, and a pharmaceutically-acceptable carrier.

In yet another aspect, the present invention provides a pharmaceutical composition including at least one compound selected from the group consisting of compounds of Formula I as described herein and a pharmaceutically-acceptable carrier or diluent.

In certain embodiments, the composition is in the form of a hydrate, solvate or pharmaceutically-acceptable salt. The composition can be administered to the subject by any suitable route of administration, including, without limitation, oral and parenteral.

The phrase “pharmaceutically-acceptable carrier” as used herein means a pharmaceutically-acceptable material, composition or vehicle, such as a liquid or solid filler, diluent, excipient, solvent, or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose, and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as butylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term “carrier” denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

As set out above, certain embodiments of the present pharmaceutical agents may be provided in the form of pharmaceutically-acceptable salts. The term “pharmaceutically-acceptable salt,” in this respect, refers to the relatively non-toxic, inorganic and organic acid salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts, and the like. See, for example, Berge et al., (1977) “Pharmaceutical Salts”, J. Pharm. Sci. 66:1-19.

The pharmaceutically-acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, or tertiary amine. Representative alkali or alkaline earth salts include the lithium, sodium, potassium, calcium, magnesium, aluminum salts, and the like. Representative organic amines useful for the formation of base addition salts include ethylamine, diethylamine, ethylenediamine, ethanolamine, diethanolamine, piperazine, and the like. See, for example, Berge et al., supra.

Wetting agents, emulsifiers, and lubricants, such as sodium lauryl sulfate, magnesium stearate, and polyethylene oxide-polybutylene oxide copolymer, as well as coloring agents, release agents, coating agents, sweetening, flavoring and perfuming agents, preservatives, and antioxidants can also be present in the compositions.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, and most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouthwashes and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules, and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also include buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying proportions, to provide the desired release profile, other polymer matrices, liposomes, and/or microspheres. They may be sterilized by, for example, filtration through a bacteria-retaining filter, or by incorporating sterilizing agents in the form of sterile solid compositions, which can be dissolved in sterile water or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

Liquid dosage forms for oral administration of the compounds of the invention include pharmaceutically-acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, EA, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-β-cyclodextrin, may be used to solubilize compounds.

Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar, and tragacanth, and mixtures thereof.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane.

Transdermal patches have the added advantage of providing controlled delivery of a compound of the present invention to the body. Such dosage forms can be made by dissolving, or dispersing the pharmaceutical agents in the proper medium. Absorption enhancers can also be used to increase the flux of the pharmaceutical agents of the invention across the skin. The rate of such flux can be controlled, by either providing a rate-controlling membrane or dispersing the compound in a polymer matrix or gel.

Ophthalmic formulations, eye ointments, powders, solutions, and the like, are also contemplated as being within the scope of this invention.

Pharmaceutical compositions of this invention suitable for parenteral administration include one or more compounds of the invention in combination with one or more pharmaceutically-acceptable sterile isotonic aqueous or nonaqueous solutions, dispersions, suspensions, or emulsions; or sterile powders which may be reconstituted into sterile injectable solutions or dispersions just prior to use, which may contain antioxidants, buffers, bacteriostats, or solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polypropylene oxide copolymers where the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly-(orthoesters) and poly-(anhydrides). Depot-injectable formulations are also prepared by entrapping the drug in liposomes or microemulsions, which are compatible with body tissue.

When the compounds of the present invention are administered as pharmaceuticals, to humans and animals, they can be given per se (neat) or as a pharmaceutical composition containing, for example, 0.1% to 99.5% (more preferably, 0.5% to 90%) of active ingredient in combination with a pharmaceutically-acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anticancer agents).

The compounds of the invention may be administered intravenously, intramuscularly, intraperitoneally, subcutaneously, topically, orally, or by other acceptable means. The compounds may be used to treat arthritic conditions in mammals (e.g., humans, livestock, and domestic animals), racehorses, birds, lizards, and any other organism which can tolerate the compounds.

The invention also provides a pharmaceutical pack or kit including one or more containers filled with one or more of the ingredients of the pharmaceutical compositions of the invention. Optionally associated with such container(s) can be a notice in the form prescribed by a governmental agency regulating the manufacture, use, or sale of pharmaceuticals or biological products, which notice reflects approval by the agency of manufacture, use, or sale for human administration.

### Administration to a Subject

In yet another aspect, the present invention provides a method for treating a condition in a mammalian species in need thereof, the method including administering to the mammalian species a therapeutically effective amount of at least one compound selected from the group consisting of compounds of Formula I, or a pharmaceutically-acceptable salt thereof, where the condition is selected from the group consisting of cancer, an immunological disorder, a central nervous system disorder, an inflammatory disorder, a gastroenterological disorder, a metabolic disorder, a cardiovascular disorder, and a kidney disease.

In some embodiments, the cancer is selected from the group consisting of biliary tract cancer, brain cancer, breast cancer, cervical cancer, choriocarcinoma, colon cancer, endometrial cancer, esophageal cancer, gastric (stomach) cancer, intraepithelial neoplasms, leukemias, lymphomas, liver cancer, lung cancer, melanoma, neuroblastomas, oral cancer, ovarian cancer, pancreatic cancer, prostate cancer, rectal cancer, renal (kidney) cancer, sarcomas, skin cancer, testicular cancer, and thyroid cancer.

In some embodiments, the inflammatory disorder is an inflammatory skin condition, arthritis, psoriasis, spondylitis, parodontitits, or an inflammatory neuropathy. In some embodiments, the gastroenterological disorder is an inflammatory bowel disease such as Crohn's disease or ulcerative colitis.

In some embodiments, the immunological disorder is transplant rejection or an autoimmune disease (e.g., rheumatoid arthritis, multiple sclerosis, systemic lupus erythematosus, or type I diabetes mellitus). In some embodiments, the central nervous system (CNS) disorder is Alzheimer's disease.

In some embodiments, the metabolic disorder is obesity or type II diabetes mellitus. In some embodiments, the cardiovascular disorder is an ischemic stroke. In some embodiments, the kidney disease is chronic kidney disease, nephritis, or chronic renal failure.

In some embodiments, the mammalian species is human.

In some embodiments, the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, inflammatory bowel disease, obesity, type II diabetes mellitus, ischemic stroke, chronic kidney disease, nephritis, chronic renal failure, and a combination thereof.

In yet another aspect, a method of blocking Kv1.3 potassium channel in a mammalian species in need thereof is described, including administering to the mammalian species a therapeutically effective amount of at least one compound of Formula I, or a pharmaceutically-acceptable salt thereof.

In some embodiments, the compounds described herein is selective in blocking the Kv 1.3 potassium channels with minimal or no off-target inhibition activities against other potassium channels, or against calcium or sodium channels. In some embodiments, the compounds described herein do not block the hERG channels and therefore have desirable cardiovascular safety profiles.

Some aspects of the invention involve administering an effective amount of a composition to a subject to achieve a specific outcome. The small molecule compositions useful according to the methods of the present invention thus can be formulated in any manner suitable for pharmaceutical use.

The formulations of the invention are administered in pharmaceutically-acceptable solutions, which may routinely contain pharmaceutically-acceptable concentrations of salt, buffering agents, preservatives, compatible carriers, adjuvants, and optionally other therapeutic ingredients.

For use in therapy, an effective amount of the compound can be administered to a subject by any mode allowing the compound to be taken up by the appropriate target cells. “Administering” the pharmaceutical composition of the present invention can be accomplished by any means known to the skilled artisan. Specific routes of administration include, but are not limited to, oral, transdermal (e.g., via a patch), parenteral injection (subcutaneous, intradermal, intramuscular, intravenous, intraperitoneal, intrathecal, etc.), or mucosal (intranasal, intratracheal, inhalation, intrarectal, intravaginal, etc.). An injection can be in a bolus or a continuous infusion.

For example the pharmaceutical compositions according to the invention are often administered by intravenous, intramuscular, or other parenteral means. They can also be administered by intranasal application, inhalation, topically, orally, or as implants; even rectal or vaginal use is possible. Suitable liquid or solid pharmaceutical preparation forms are, for example, aqueous or saline solutions for injection or inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops, or preparations with protracted release of active compounds in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer R (1990) Science 249:1527-33, which is incorporated herein by reference.

The concentration of compounds included in compositions used in the methods of the invention can range from about 1 nM to about 100 μM. Effective doses are believed to range from about 10 picomole/kg to about 100 micromole/kg.

The pharmaceutical compositions are preferably prepared and administered in dose units. Liquid dose units are vials or ampoules for injection or other parenteral administration. Solid dose units are tablets, capsules, powders, and suppositories. For treatment of a patient, different doses may be necessary depending on activity of the compound, manner of administration, purpose of the administration (i.e., prophylactic or therapeutic), nature and severity of the disorder, and age and body weight of the patient. The administration of a given dose can be carried out both by single administration in the form of an individual dose unit or else several smaller dose units. Repeated and multiple administration of doses at specific intervals of days, weeks, or months apart are also contemplated by the invention.

The compositions can be administered per se (neat) or in the form of a pharmaceutically-acceptable salt. When used in medicine the salts should be pharmaceutically acceptable, but non-pharmaceutically-acceptable salts can conveniently be used to prepare pharmaceutically-acceptable salts thereof. Such salts include, but are not limited to, those prepared from the following acids: hydrochloric, hydrobromic, sulphuric, nitric, phosphoric, maleic, acetic, salicylic, TsOH (p-toluene sulphonic acid), tartaric, citric, methane sulphonic, formic, malonic, succinic, naphthalene-2-sulphonic, and benzene sulphonic acids. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v), and phosphoric acid and a salt (0.8-2% w/v). Suitable preservatives include benzalkonium chloride (0.003-0.03% w/v); chlorobutanol (0.3-0.9% w/v); parabens (0.01-0.25% w/v), and thimerosal (0.004-0.02% w/v).

Compositions suitable for parenteral administration conveniently include sterile aqueous preparations, which can be isotonic with the blood of the recipient. Among the acceptable vehicles and solvents are water, Ringer's solution, phosphate buffered saline, and isotonic sodium chloride solution. In addition, sterile, fixed oils are conventionally employed as a solvent or suspending medium. For this purpose, any bland fixed mineral or non-mineral oil may be employed including synthetic mono- or diglycerides. In addition, fatty acids such as oleic acid find use in the preparation of injectables. Carrier formulations suitable for subcutaneous, intramuscular, intraperitoneal, intravenous, etc. administrations can be found in Remington's Pharmaceutical Sciences, Mack Publishing Company, Easton, Pa.

The compounds useful in the invention can be delivered in mixtures of more than two such compounds. A mixture can further include one or more adjuvants in addition to the combination of compounds.

A variety of administration routes is available. The particular mode selected will depend, of course, upon the particular compound selected, the age and general health status of the subject, the particular condition being treated, and the dosage required for therapeutic efficacy. The methods of this invention, generally speaking, can be practiced using any mode of administration that is medically acceptable, meaning any mode that produces effective levels of response without causing clinically unacceptable adverse effects. Preferred modes of administration are discussed above.

The compositions can conveniently be presented in unit dosage form and can be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product.

Other delivery systems can include time-release, delayed release, or sustained-release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids, or neutral fats such as mono-di- and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### Assays for Effectiveness of Kv1.3 Potassium Channel Blockers

In some embodiments, the compounds as described herein are tested for their activities against Kv1.3 potassium channel. In some embodiments, the compounds as described herein are tested for their Kv1.3 potassium channel electrophysiology. In some embodiments, the compounds as described herein are tested for their hERG electrophysiology.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification, and guidance which can be adapted to the practice of this invention in its various embodiments and equivalents thereof.

### EXAMPLES

Examples 1-7 describe various intermediates used in the syntheses of representative compounds of Formula I disclosed herein.

### Example 1. Intermediate 1 (2-bromo-3,4-dichloro-1-methoxybenzene) and Intermediate 2 (1-bromo-4,5-dichloro-2-methoxybenzene)

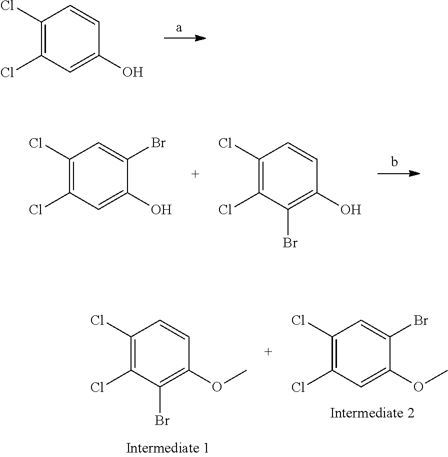

Step a:

To a stirred solution of 3,4-dichlorophenol (100.00 g, 613.49 mmol) in DCM (1000 mL) was added Br₂ (98.04 g, 613.49 mmol) dropwise at 0° C. under nitrogen atmosphere. The reaction solution was stirred for 16 h at room temperature under nitrogen atmosphere. The reaction was quenched with saturated aq. Na₂S₂O₃ (500 mL) at 0° C. The resulting mixture was extracted with EA (6×400 mL). The combined organic layers were washed with brine (2×400 mL) and dried over anhydrous Na₂SO₄. After filtration, the filtrate was concentrated under reduced pressure to afford a mixture of 2-bromo-4,5-dichlorophenol and 2-bromo-3,4-dichlorophenol (100 g, crude) as a yellow oil. The crude product was used in the next step directly without further purification.

To a crude mixture of 2-bromo-4,5-dichlorophenol and 2-bromo-3,4-dichlorophenol (32 g, 125.04 mmol, 1 equiv.) and K₂CO₃ (54.9 g, 396.87 mmol, 3 equiv.) in ACN (210 mL) was added Mel (16.5 mL, 116.05 mmol, 2 equiv.) dropwise at 0° C. The reaction mixture was stirred at 50° C. for 4 h. The reaction mixture was filtered and concentrated. The residue was purified by silica gel column chromatography, eluted with PE to afford Intermediate 1 (2-bromo-3,4-dichloro-1-methoxybenzene) (8.7 g, 25.7%) as a white solid: ¹H NMR (300 MHz, CDCl₃) δ 7.40 (dd, J=9.0, 1.1 Hz, 1H), 6.79 (d, J=8.9 Hz, 1H), 3.92 (s, 3H); and Intermediate 2 (1-bromo-4,5-dichloro-2-methoxybenzene) (24.3 g, 71.77%) as a white solid: ¹H NMR (300 MHz, CDCl₃) δ 7.64 (s, 1H), 6.99 (s, 1H), 3.91 (s, 3H).

### Example 2. Intermediate 3 ((2,3-dichloro-6-methoxyphenyl)boronic acid)

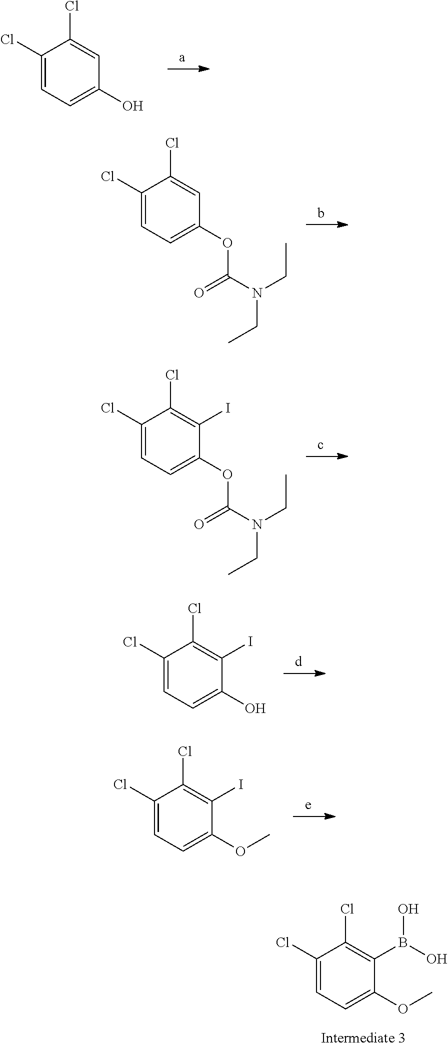

Step a:

To a stirred solution of 3,4-dichlorophenol (120 g, 0.74 mol) in THE (400 mL) was added NaOH (75 g, 1.88 mol) in portions at room temperature under nitrogen atmosphere, followed by stirring for 30 min. To this was added N,N-diethylcarbamoyl chloride (150 g, 1.11 mol) over 40 min, followed by stirring for 15 h. The reaction mixture was poured into water (1.5 L) and extracted with PE (2×800 mL). The combined organic phase was washed with brine (500 mL) and dried over Na₂SO₄. After filtration, the filtrate was concentrated under reduced pressure to afford 3,4-dichlorophenyl N,N-diethylcarbamate as a yellow oil (213 g, crude): LCMS (ESI) calculated for C₁₁H₁₃Cl₂NO₂ [M+H]⁺: 262, 264 (3:2), found 262, 264 (3:2); ¹H NMR (400 MHz, CDCl₃) δ 7.43 (d, J=8.8 Hz, 1H), 7.30 (d, J=2.7 Hz, 1H), 7.03 (dd, J=8.8, 2.7 Hz, 1H), 3.50-3.34 (m, 4H), 1.32-1.17 (m, 6H).

Step b:

To a solution of DIPA (32 g, 0.32 mol) in THE (400 mL) was added dropwise n-BuLi (131 mL, 0.33 mmol) at −65° C. under nitrogen atmosphere. The resulting mixture was stirred for 1 h. To this was added a solution of 3,4-dichlorophenyl N,N-diethylcarbamate (77 g, 0.29 mol) in THE (200 mL) dropwise, followed by stirring for 1 h. To this was added a solution of 12 (82 g, 0.32 mol) in THE (200 mL) dropwise over 1 h. The resulting mixture was stirred for an additional 30 min at −65° C. The reaction was quenched by the addition of aq. NH₄Cl (300 mL) at room temperature. The resulting mixture was extracted with EA (3×400 mL). The combined organic layers were washed with brine (500 mL) and dried over anhydrous Na₂SO₄. After filtration, the filtrate was concentrated under reduced pressure. Three additional batches (3×77 g of 3,4-dichlorophenyl N,N-diethylcarbamate) were similarly reacted and worked up, then combined with the previous one. The resulting residue was slurried in PE (500 mL), and then filtered to afford 300 g of 3,4-dichloro-2-iodophenyl N,N-diethylcarbamate. The filtrate was purified with silica gel column chromatography, eluted with PE/EA (50/1) to afford another 75 g of pure product. 3,4-dichloro-2-iodophenyl N,N-diethylcarbamate (375 g, 83% over 2 steps) was obtained as an off-white solid: LCMS (ESI) calculated for C₁₁H₁₂Cl₂INO₃ [M+H]⁺: 388, 390 (3:2), found 388, 390 (3:2); ¹H NMR (400 MHz, CDCl₃) δ 7.48 (d, J=8.8 Hz, 1H), 7.08 (d, J=8.8 Hz, 1H), 3.55 (q, J=7.2 Hz, 2H), 3.42 (q, J=7.1 Hz, 2H), 1.34 (t, J=7.1 Hz, 3H), 1.25 (t, J=7.1 Hz, 3H).

Step c:

To a stirred solution of 3,4-dichloro-2-iodophenyl N,N-diethylcarbamate (200 g, 0.52 mol) in EtOH (1.50 L) was added NaOH (165 g, 4.1 mol) at room temperature. The resulting mixture was stirred for 1 h at 80° C. under nitrogen atmosphere. The reaction mixture was concentrated under reduced pressure. The residue was diluted with ice water (1.5 L). The mixture was then acidified with aq. HCl (6 N) to pH=3. The resulting mixture was extracted with EA (3×1 L). The combined organic layers were washed with brine (800 mL) and dried over anhydrous Na₂SO₄. After filtration, the filtrate was concentrated under reduced pressure to afford 3,4-dichloro-2-iodophenol as a brown oil (202 g, crude): LCMS (ESI) calculated for C₆H₃Cl₂IO [M−H]⁻: 287, 289 (3:2), found 287, 289 (3:2).

Step d:

To a stirred solution of 3,4-dichloro-2-iodophenol (220 g, 0.76 mol) in DMF (700 mL) was added K₂CO₃ (210 g, 1.52 mol) and Mel (119 g, 0.84 mol). The resulting mixture was stirred for 5 h at room temperature. Another batch (100 g of 3,4-dichloro-2-iodophenol) was similarly reacted and combined with the previous one. The resulting mixture was diluted with water (5 L) at room temperature. The resulting mixture was then extracted with EA (3×1 L). The combined organic layers were washed with brine (4×400 mL) and dried over anhydrous Na₂SO₄. After filtration, the filtrate was concentrated under reduced pressure. The residue was slurried in PE (300 mL), and then filtered to afford 128 g of desired product. The filtrate was purified by silica gel column chromatography, eluted with PE/EA (40/1) to afford an additional 64 g of desired product. 1,2-dichloro-3-iodo-4-methoxybenzene (192 g, 78% over 2 steps) was obtained as a light yellow solid: ¹H NMR (400 MHz, CDCl₃) δ 7.44 (d, J=8.9 Hz, 1H), 6.70 (d, J=8.9 Hz, 1H), 3.91 (s, 3H).

Step e:

To a solution of 1,2-dichloro-3-iodo-4-methoxybenzene (100 g, 0.33 mol) in THF (1.2 L) was added i-PrMgCl (182 mL, 0.36 mol) dropwise at 0° C. under nitrogen atmosphere. The reaction mixture was then stirred at 0° C. for 1 h. B(OMe)₃ (86 g, 0.83 mol) was added dropwise at 0° C. Then, the reaction mixture was allowed to warm to room temperature over 1 h and stirred at room temperature for an additional 1 h. Then, aq. H₂SO₄ (5%, 500 mL) was added dropwise at 0° C. The reaction mixture was stirred at room temperature for 30 min. The mixture was extracted with EA (2×500 mL). The organic layers were combined, washed with brine (500 mL) and dried over anhydrous Na₂SO₄. After filtration, the filtrate was concentrated. The residue was stirred in DCM (200 mL), and then filtered to afford Intermediate 3 ((2,3-dichloro-6-methoxyphenyl)boronic acid) as an off-white solid (55 g, 76%): LCMS (ESI) calculated for C₁₅H₁₆Cl₂N₂O₄ [M−H]⁻: 219, 221 (3:2), found 219, 221 (3:2); ¹H NMR (400 MHz, CDCl₃) δ 7.48 (d, J=8.8 Hz, 1H), 6.82 (d, J=8.9 Hz, 1H), 5.65 (s, 2H), 3.89 (s, 3H).

### Example 3. Intermediate 4 (1-tert-butyl 2-methyl (2S)-4-(trifluoromethanesulfonyloxy)-2,3-dihydropyrrole-1,2-dicarboxylate)

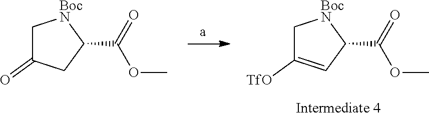 [SMILES: COC(=O)[C@@H]1CC(=O)CN1C(=O)OC(C)(C)C ; COC(=O)[C@@H]1C=C(OS(=O)(=O)C(F)(F)F)CN1C(=O)OC(C)(C)C]

Step a:

To a solution of 1-tert-butyl 2-methyl (2S)-4-oxopyrrolidine-1,2-dicarboxylate (2.0 g, 8.22 mmol) in THE (15 mL) was added LiHMDS (9.87 mL, 9.87 mmol, 1 M in THF) dropwise over 10 min at −65° C. under nitrogen atmosphere. After stirring for 0.5 h, 1,1,1-trifluoro-N-phenyl-N-trifluoromethanesulfonylmethanesulfonamide (4.41 g, 12.35 mmol) in THE (5 mL) was added dropwise at −65° C. The resulting solution was stirred for 1 h at room temperature under nitrogen atmosphere. The reaction was quenched with saturated aq. NH₄Cl (50 mL) at room temperature. The resulting mixture was extracted with EA (3×50 mL). The combined organic layers were washed with brine (3×50 mL) and dried over anhydrous Na₂SO₄. After filtration, the filtrate was concentrated under reduced pressure to afford Intermediate 4 (1-tert-butyl 2-methyl (2S)-4-(trifluoromethanesulfonyloxy)-2,3-dihydropyrrole-1,2-dicarboxylate) as a yellow oil (3 g, crude), which was used in the next step directly without further purification: LCMS (ESI) calculated for C₁₂H₁₆F₃NO₇S [M+H−56]⁺ 320, found 320.

### Example 4. Intermediate 5 (1-[4-(2,3-dichloro-6-methoxyphenyl)piperidin-2-yl]methanamine bis(trifluoroacetic acid))

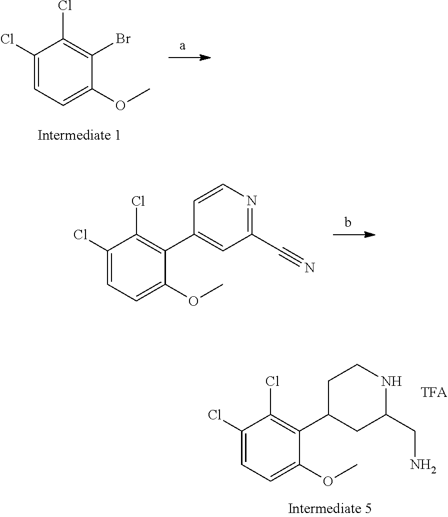 [SMILES: COc1ccc(Cl)c(Cl)c1Br ; COc1ccc(Cl)c(Cl)c1-c1ccnc(C#N)c1 ; COc1ccc(Cl)c(Cl)c1C1CCNC(CN)C1]

Step a:

To a solution of Intermediate 1 (Example 1) (5.00 g, 16.51 mmol) and 4-(4,4,5,5-tetramethyl-1,3,2-dioxaborolan-2-yl)pyridine-2-carbonitrile (3.80 g, 16.51 mmol) in 1,4-dioxane (80 mL) and H₂O (20 mL) were added Na₂CO₃ (5.25 g, 49.53 mmol) and Pd(dppf)Cl₂ CH₂Cl₂ (0.67 g, 0.83 mmol) under nitrogen atmosphere. The reaction mixture was stirred 80° C. for 3 h under nitrogen atmosphere. The reaction mixture was poured into water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (3/1) to afford 4-(2,3-dichloro-6-methoxyphenyl)pyridine-2-carbonitrile as an off-white solid (3.00 g, 65%): LCMS (ESI) calculated for C₁₃H₁₈Cl₂N₂O [M+H]⁺: 279, 281 (3:2), found 279, 281 (3:2); ¹H NMR (400 MHz, CDCl₃) δ 8.80 (dd, J=5.0, 0.9 Hz, 1H), 7.64 (s, 1H), 7.54 (d, J=8.9 Hz, 1H), 7.46 (dd, J=5.0, 1.7 Hz, 1H), 6.92 (d, J=9.0 Hz, 1H), 3.77 (s, 3H).

To a stirred mixture of 4-(2,3-dichloro-6-methoxyphenyl)pyridine-2-carbonitrile (3.00 g, 10.75 mmol) in MeOH (400 mL) and conc. HCl (12 M, 40.00 mL) was added PtO₂ (0.50 g, 2.16 mmol) in portions at room temperature. The reaction mixture was degassed and stirred at 30° C. under hydrogen atmosphere (50 atm) for 48 h. The mixture was filtered and the filtrate was concentrated under reduced pressure. The residue was purified by reverse phase chromatography, eluted with 40% ACN in water (plus 0.05% TFA) to afford Intermediate 5 (1-[4-(2,3-dichloro-6-methoxyphenyl)piperidin-2-yl]methanamine bis(trifluoroacetic acid)) as an off-white solid (2.8 g, 50%): LCMS (ESI) calculated for C₁₃H₁₈Cl₂N₂O [M+H]⁺: 289, 291 (3:2), found 289, 291 (3:2); ¹H NMR (400 MHz, CD₃OD) δ 7.36 (d, J=9.0 Hz, 1H), 6.95 (d, J=9.0 Hz, 1H), 3.85 (s, 3H), 3.66-3.52 (m, 1H), 3.25-3.16 (m, 1H), 2.83-2.73 (m, 1H), 2.73-2.62 (m, 3H), 2.48-2.33 (m, 1H), 2.16-1.98 (m, 1H), 1.58 (dd, J=31.4, 12.8 Hz, 2H).

### Example 5. Intermediate 6 (8R,9aS)-8-(2,3-dichloro-6-hydroxyphenyl)-octahydropyrido[1,2-a]pyrazin-4-one)

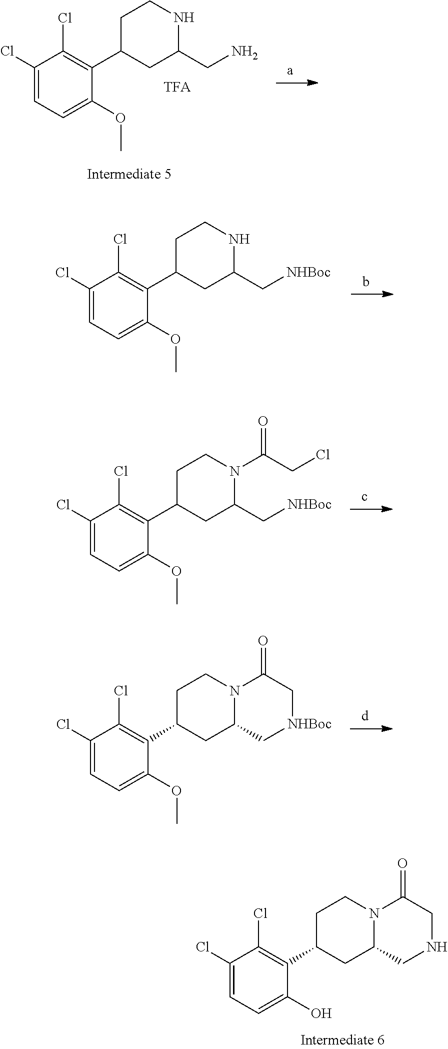 [SMILES: COc1ccc(Cl)c(Cl)c1C1CCNC(CN)C1 ; COc1ccc(Cl)c(Cl)c1C1CCNC(CNC(=O)OC(C)(C)C)C1 ; COc1ccc(Cl)c(Cl)c1C1CCN(C(=O)CCl)C(CNC(=O)OC(C)(C)C)C1 ; COc1ccc(Cl)c(Cl)c1[C@@H]1CCN(C(C)=O)[C@H](CNC(=O)OC(C)(C)C)C1 ; O=C1CNC[C@@H]2C[C@H](c3c(O)ccc(Cl)c3Cl)CCN12]

Step a:

To a stirred mixture of 1-[4-(2,3-dichloro-6-methoxyphenyl)piperidin-2-yl]methanamine trifluoroacetic acid (Intermediate 5, Example 4) (1.00 g, 2.59 mmol) and TEA (0.75 g, 7.50 mmol) in DCM (15.00 mL) was added Boc₂O (0.43 g, 2.00 mmol) at −50° C. under nitrogen atmosphere. The resulting mixture was stirred for 1 h at −50° C. under nitrogen atmosphere and then quenched with NH₃.H₂O (2 mL), diluted with water (20 mL), and extracted with EA (3×20 mL). The combined organic layers were washed with brine (3×30 mL) and dried over anhydrous Na₂SO₄. After filtration, the filtrate was concentrated under reduced pressure. The residue was purified by silica gel column chromatography, eluted with hexane/EA (1/1) to afford tert-butyl N-[[4-(2,3-dichloro-6-methoxyphenyl)piperidin-2-yl]methyl]carbamate as an off-white solid (0.6 g, 62%): LCMS (ESI) calculated for C₁₈H₂₆Cl₂N₂O₃ [M+H]⁺: 389, 401 (3:2), found 389, 401 (3:2).

Step b:

To a solution of tert-butyl N-[[4-(2,3-dichloro-6-methoxyphenyl)piperidin-2-yl]methyl]carbamate (0.60 g, 1.54 mmol) and TEA (0.47 g, 4.62 mmol) in DCM (10 mL) was added chloroacetyl chloride (0.19 g, 2.00 mmol) at 0° C., then the reaction was stirred at room temperature for 1 h. The resulting reaction mixture was concentrated to afford tert-butyl N-[[1-(2-chloroacetyl)-4-(2,3-dichloro-6-methoxyphenyl)piperidin-2-yl]methyl]carbamate as a yellow oil (0.7 g, crude): LCMS (ESI) calculated for C₂₀H₂₇C₁₃N₂O₄ [M+H]⁺: 465, 467 (1:1), found 465, 467 (1:1).

Step c:

To a solution of tert-butyl N-[[1-(2-chloroacetyl)-4-(2,3-dichloro-6-methoxyphenyl)piperidin-2-yl]methyl]carbamate (0.70 g, 1.50 mmol) in DMF (10 mL) was added Cs₂CO₃ (0.98 g, 3.00 mmol) at room temperature. The reaction mixture was stirred at 50° C. for 16 h, diluted with water (20 mL) and then extracted with EA (3×20 mL). The combined organic layers were washed with brine (3×20 mL) and dried over anhydrous Na₂SO₄. After filtration, the filtrate was concentrated under reduced pressure. The residue was purified by silica gel column chromatography, eluted with PE/EA (1:1) to afford tert-butyl 8-(2,3-dichloro-6-methoxyphenyl)-4-oxo-hexahydro-1H-pyrido[1,2-a]pyrazine-2-carboxylate as a yellow oil (0.30 g, 46%). Tert-butyl 8-(2,3-dichloro-6-methoxyphenyl)-4-oxo-hexahydro-1H-pyrido[1,2-a]pyrazine-2-carboxylate (0.30 g, 0.70 mmol) was separated by prep-chiral HPLC with following conditions: Column: CHIRALPAK IE, 2×25 cm, Sum; Mobile Phase A: Hex-HPLC, Mobile Phase B: EtOH-HPLC; Flow rate: 20 mL/min; Gradient: 30% B to 30% B in 13 min; Detector: UV 254/210 nm; Retention time: RT1:9.048 min; RT2:11.244 min. The faster-eluting enantiomer was obtained as tert-butyl (8S,9aR)-8-(2,3-dichloro-6-methoxyphenyl)-4-oxo-hexahydro-1H-pyrido[1,2-a]pyrazine-2-carboxylate at 9.048 min as a yellow oil (0.12 g, 18%): LCMS (ESI) calculated for C₂₀H₂₆Cl₂N₂O₄ [M+H]⁺: 429, 431 (3:2), found 429, 431 (3:2). The slower-eluting enantiomer was obtained as tert-butyl (8R,9aS)-8-(2,3-dichloro-6-methoxyphenyl)-4-oxo-hexahydro-1H-pyrido[1,2-a]pyrazine-2-carboxylate at 11.244 min as a yellow oil (0.12 g, 18%): LCMS (ESI) calculated for C₂₀H₂₆Cl₂N₂O₄ [M+H]⁺: 429, 431 (3:2), found 429, 431 (3:2). ¹H NMR (400 MHz, CDCl₃) δ 7.31 (d, J=8.9 Hz, 1H), 6.75 (d, J=8.9 Hz, 1H), 5.32 (s, 1H), 4.92-4.80 (m, 1H), 4.27 (d, J=18.4 Hz, 1H), 4.17-3.88 (m, 2H), 3.80 (s, 3H), 3.77-3.59 (m, 1H), 3.59-3.47 (m, 1H), 2.73-2.62 (m, 1H), 2.46-2.07 (m, 2H), 1.71-1.64 (m, 2H), 1.50 (s, 9H).

Step d:

To a solution of tert-butyl (8R,9aS)-8-(2,3-dichloro-6-methoxyphenyl)-4-oxo-hexahydro-1H-pyrido[1,2-a]pyrazine-2-carboxylate (0.12 g, 0.279 mmol) in DCM (3 mL) was added BBr₃ (0.13 mL, 0.527 mmol) dropwise at 0° C. The reaction mixture was stirred at room temperature for 3 h, quenched with water (1 mL), diluted with NaHCO₃ (sat. 10 mL) and then extracted with EA (3×20 mL). The combined organic layers were concentrated under vacuum. The residue was purified by Prep-HPLC with the following conditions: Column: XBridge Shield RP18 OBD Column, 30×150 mm, 5 μm; Mobile Phase A: water (10 mM ammonium formate), Mobile Phase B: ACN; Flow rate: 60 mL/min; Gradient: 25% B to 45% B in 7 min; Detector: UV 254/210 nm; Retention time: 6.5 min. The fractions containing the desired product were combined and concentrated under reduced pressure to afford Intermediate 6 ((8R,9aS)-8-(2,3-dichloro-6-hydroxyphenyl)-octahydropyrido[1,2-a]pyrazin-4-one) as an off-white solid (65.1 mg, 74%): LCMS (ESI) calculated for C₁₄H₁₆Cl₂N₂O₂ [M+H]⁺: 315, 317 (3:2), found 315, 317 (3:2). ¹H NMR (400 MHz, Methanol-d4) δ 7.20 (d, J=8.7 Hz, 1H), 6.71 (d, J=8.8 Hz, 1H), 4.83-4.70 (m, 1H), 3.79-3.63 (m, 1H), 3.60-3.49 (m, 1H), 3.43 (s, 2H), 3.24 (dd, J=13.4, 5.1 Hz, 1H), 2.86-2.61 (m, 2H), 2.56-2.32 (m, 2H), 1.72-1.58 (m, 2H).

### Example 6. Intermediate 7 (1-tert-butyl 2-methyl (2S,4R)-4-(2,3-dichloro-6-methoxyphenyl)pyrrolidine-1,2-dicarboxylate)

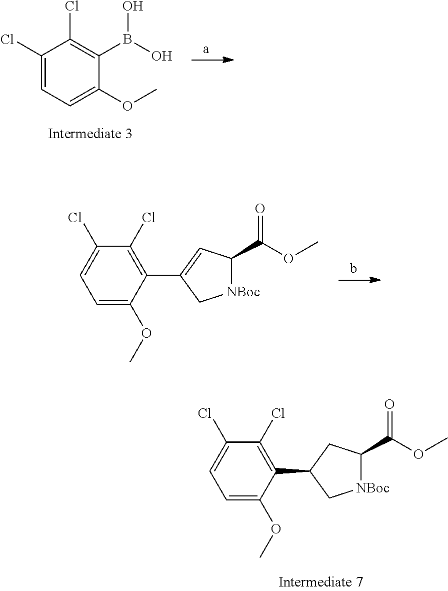 [SMILES: COc1ccc(Cl)c(Cl)c1B(O)O ; COC(=O)[C@@H]1C=C(c2c(OC)ccc(Cl)c2Cl)CN1C(=O)OC(C)(C)C ; COC(=O)[C@@H]1C[C@H](c2c(OC)ccc(Cl)c2Cl)CN1C(=O)OC(C)(C)C]

Step a:

To a stirred solution of 1-tert-butyl 2-methyl 4-(trifluoromethanesulfonyloxy)-2,3-dihydropyrrole-1,2-dicarboxylate (Intermediate 4, Example 3) (3.09 g, 8.23 mmol), 2,3-dichloro-6-methoxyphenyl)boronic acid (Intermediate 3, Example 2) (1.40 g, 6.34 mmol) and Na₂CO₃ (2.02 g, 19.06 mmol) in dioxane (15 mL) and H₂O (3 mL) was added Pd(dppf)Cl₂.CH₂Cl₂ (0.10 g, 0.12 mmol) under nitrogen atmosphere. The resulting mixture was stirred for 4 h at 80° C. under nitrogen atmosphere. The reaction was diluted with EA (50 mL) and water (50 mL). The aqueous solution was extracted with EA (3×50 mL). The combined organic layers were washed with brine (3×30 mL) and dried over anhydrous Na₂SO₄. After filtration, the filtrate was concentrated under reduced pressure. The residue was purified by silica gel column chromatography, eluted with PE/EA (4/1) to afford 1-tert-butyl 2-methyl (2S)-4-(2,3-dichloro-6-methoxyphenyl)-2,3-dihydropyrrole-1,2-dicarboxylate as a light yellow oil (1.30 g, 51%): LCMS (ESI) calculated for C₁₈H₂₁Cl₂NO₅ [M+H]⁺ 402, 404 (3:2), found 402, 404 (3:2); ¹H NMR (400 MHz, CD₃OD) δ 7.48 (d, J=8.9 Hz, 1H), 7.00 (d, J=9.0 Hz, 1H), 5.82-5.64 (m, 1H), 5.27-5.11 (m, 1H), 4.50-4.21 (m, 2H), 3.93-3.74 (m, 6H), 1.47 (d, J=15.9 Hz, 9H).

Step b:

A solution of 1-tert-butyl 2-methyl (2S)-4-(2,3-dichloro-6-methoxyphenyl)-2,5-dihydropyrrole-1,2-dicarboxylate (1.30 g, 3.23 mmol) and PtO₂ (0.22 g, 0.970 mmol) in HOAc (8 mL) was stirred for 16 h at room temperature under hydrogen atmosphere (1.5 atm). The reaction was filtered and the filtrate was concentrated under reduced pressure to afford Intermediate 7 (1-tert-butyl 2-methyl (2S,4R)-4-(2,3-dichloro-6-methoxyphenyl)pyrrolidine-1,2-dicarboxylate) as a light yellow oil (1.30 g, 99%): LCMS (ESI) calculated for C₁₈H₂₃Cl₂NO₅ [M+H]⁺404, 406 (3:2), found 404, 406 (3:2); ¹H NMR (400 MHz, CD₃OD) δ 7.42 (d, J=9.0, 1.2 Hz, 1H), 6.98 (d, J=9.0 Hz, 1H), 4.48-4.38 (m, 1H), 4.30-4.18 (m, 1H), 3.86 (d, J=2.2 Hz, 3H), 3.80 (d, J=3.7 Hz, 3H), 3.71-3.57 (m, 1H), 3.35-3.29 (m, 1H), 2.70-2.41 (m, 2H), 1.47 (d, J=14.2 Hz, 9H).

### Example 7. Intermediate 8 ((7R,8aS)-7-(2,3-dichloro-6-hydroxyphenyl)-hexahydro-1H-pyrrolo[1,2-a]pyrazin-4-one hydrobromide)

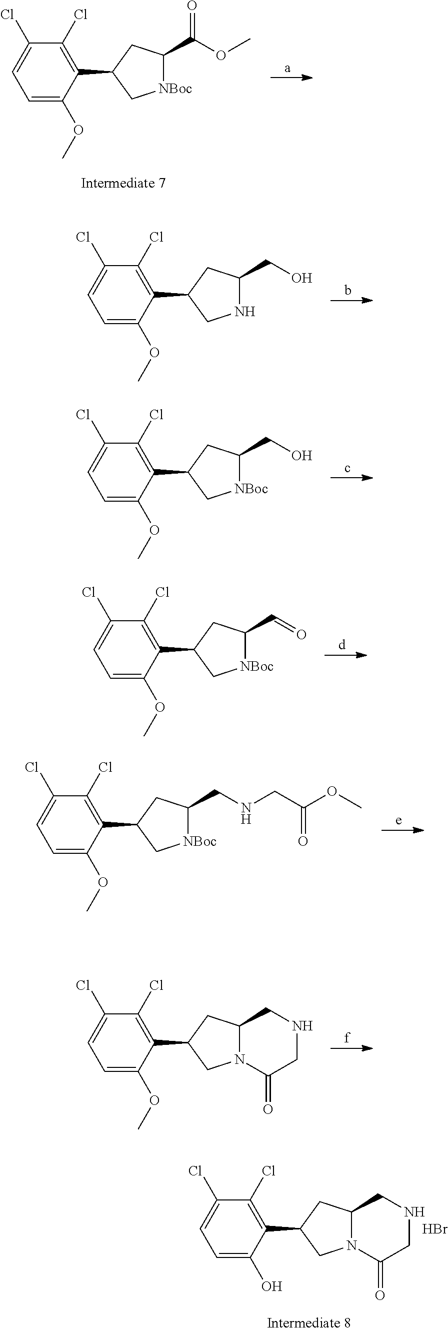

Step a:

To a stirred solution of 1-tert-butyl 2-methyl (2S,4R)-4-(2,3-dichloro-6-methoxyphenyl)pyrrolidine-1,2-dicarboxylate (Intermediate 7, Example 6) (6.00 g, 14.84 mmol) in THE (20 mL) was added BH₃.Me₂S (2.97 mL, 29.68 mmol) at room temperature under nitrogen atmosphere. The reaction was stirred at 70° C. for 2 h. The reaction was quenched with MeOH (5 mL) at 0° C. and then aq. HCl (6 N, 5 mL) was added. The resulting solution was stirred at 70° C. for 1 h. The reaction was concentrated under reduced pressure to afford [(2S,4R)-4-(2,3-dichloro-6-methoxyphenyl)pyrrolidin-2-yl]methanol hydrochloride as a light-yellow oil (5.0 g, crude), which was used to next step directly without further purification: LCMS (ESI) calculated for C₁₂H₁₅Cl₂NO₂ [M+H]⁺ 276, 278 (3:2), found 276, 278 (3:2).

Step b:

To a stirred solution of [(2S,4R)-4-(2,3-dichloro-6-methoxyphenyl)pyrrolidin-2-yl]methanol hydrochloride (5.0 g, 15.99 mmol) and TEA (3.22 g, 31.82 mmol) in DCM (20 mL) was added Boc₂O (3.80 g, 17.41 mmol) at room temperature. The reaction was stirred at room temperature for 1 h. The reaction solution was diluted with EA (50 mL) and water (50 mL). The aqueous solution was extracted with EA (3×50 mL). The combined organic layers were washed with brine (2×50 mL) and dried over anhydrous Na₂SO₄. After filtration, the filtrate was concentrated under reduced pressure. The residue was purified by reverse phase chromatography, eluted with 75% ACN in water with 10 mmol/L NH₄HCO₃ to afford tert-butyl (2S,4R)-4-(2,3-dichloro-6-methoxyphenyl)-2-(hydroxymethyl)pyrrolidine-1-carboxylate as an off-white solid (1.90 g, 32% over two steps): LCMS (ESI) calculated for C₁₇H₂₃Cl₂NO₄ [M+H]⁺ 376, 378 (3:2), found 376, 378 (3:2); ¹H NMR (400 MHz, CD₃OD) δ 7.39 (d, J=8.9 Hz, 1H), 6.97 (d, J=9.0 Hz, 1H), 4.62 (s, 1H), 4.08-3.91 (m, 1H), 3.87 (s, 3H), 3.85-3.63 (m, 4H), 2.77-2.46 (m, 1H), 2.28-2.11 (m, 1H), 1.50 (d, J=11.2 Hz, 9H).

Step c:

To a stirred solution of tert-butyl (2S,4R)-4-(2,3-dichloro-6-methoxyphenyl)-2-(hydroxymethyl)pyrrolidine-1-carboxylate (1.90 g, 5.050 mmol) in DCM (10 mL) was added Dess-Martin periodinane (2.57 g, 6.06 mmol) at room temperature. The reaction was stirred for 1 h and then quenched with saturated aq. Na₂S₂O₃ (30 mL). The mixture was extracted with EA (3×30 mL). The combined organic layers were washed with saturated aq. NaHCO₃ (3×30 mL), and brine (2×50 mL) and dried over anhydrous Na₂SO₄. After filtration, the filtrate was concentrated under reduced pressure to afford tert-butyl (2S,4R)-4-(2,3-dichloro-6-methoxyphenyl)-2-formylpyrrolidine-1-carboxylateas a light yellow oil (1.9 g, crude), which was used in next step directly without further purification: LCMS (ESI) calculated for C₁₇H₂₁Cl₂NO₄ [M+H]⁺ 374, 376 (3:2), found 374, 376 (3:2).

Step d:

To a stirred solution of tert-butyl (2S,4R)-4-(2,3-dichloro-6-methoxyphenyl)-2-formylpyrrolidine-1-carboxylate (1.90 g, 5.08 mmol) and methyl 2-aminoacetate hydrochloride (0.96 g, 7.65 mmol) in DCM (20 mL) were added TEA (1.28 g, 12.65 mmol) and NaBH(AcO)₃ (2.15 g, 10.14 mmol) at room temperature. The reaction was stirred for 2 h and then quenched with water (50 mL). The mixture was extracted with EA (3×50 mL). The combined organic layer was washed with brine (2×50 mL) and dried over anhydrous Na₂SO₄. After filtration, the filtrate was concentrated under reduced pressure. The residue was purified by reverse phase chromatography, eluted with 45% ACN in water (0.05% TFA) to afford tert-butyl (2S,4R)-4-(2,3-dichloro-6-methoxyphenyl)-2-[[(2-methoxy-2-oxoethyl)amino]methyl]pyrrolidine-1-carboxylate as a yellow foam (1.80 g, 79% overall two steps): LCMS (ESI) calculated for C₂₀H₂₈Cl₂N₂O₅ [M+H]⁺ 447, 449 (3:2), found 447, 449 (3:2); ¹H NMR (400 MHz, CD₃OD) δ 7.46 (d, J=8.7 Hz, 1H), 7.03 (d, J=9.0 Hz, 1H), 4.28 (s, 1H), 4.23-3.95 (m, 3H), 3.90 (d, J=9.3 Hz, 6H), 3.87-3.71 (m, 2H), 3.41-3.35 (m, 2H), 2.49-2.32 (m, 2H), 1.53 (s, 9H).

Step e:

To a stirred solution of tert-butyl (2S,4R)-4-(2,3-dichloro-6-methoxyphenyl)-2-[[(2-methoxy-2-oxoethyl)amino]methyl]pyrrolidine-1-carboxylate (1.80 g, 4.02 mmol) in DCM (15 mL) was added TFA (3 mL) at room temperature. The reaction was stirred at room temperature for 1 h and then concentrated under reduced pressure. The resulting mixture was dissolved in EtOH (10 mL) and TEA (1.23 g, 12.16 mmol) was added into it. The reaction was stirred at 70° C. for 1 h. The reaction was diluted with water (50 mL). The mixture was extracted with EA (3×50 mL). The combined organic layer was washed with brine (2×50 mL) and dried over anhydrous Na₂SO₄. After filtration, the filtrate was concentrated under reduced pressure to afford (7R,8aS)-7-(2,3-dichloro-6-methoxyphenyl)-hexahydro-1H-pyrrolo[1,2-a]pyrazin-4-one as a yellow oil (1.10 g, 87%): LCMS (ESI) calculated for C₁₄H₁₆Cl₂N₂O₂ [M+H]⁺ 315, 317 (3:2), found 315, 317 (3:2); ¹H NMR (400 MHz, CD₃OD) δ 7.43 (d, J=9.0 Hz, 1H), 7.00 (d, J=9.0 Hz, 1H), 4.40-4.26 (m, 1H), 4.15-4.05 (m, 1H), 3.90-3.79 (m, 4H), 3.59-3.46 (m, 2H), 3.43-3.39 (m, 1H), 3.39-3.36 (m, 1H), 2.61 (dd, J=13.0, 10.3 Hz, 1H), 2.26-2.06 (m, 2H).

Step f:

To a stirred solution of (7R,8aS)-7-(2,3-dichloro-6-methoxyphenyl)-hexahydro-1H-pyrrolo[1,2-a]pyrazin-4-one (1.10 g, 3.49 mmol) in DCM (10 mL) was added BBr₃ (3.50 g, 13.97 mmol) dropwise at room temperature. The reaction was stirred for 2 h and then quenched with MeOH (10 mL). The mixture was filtered and the filter cake was washed with EA (3×5 mL), and dried under reduced pressure to afford Intermediate 8 ((7R,8aS)-7-(2,3-dichloro-6-hydroxyphenyl)-hexahydro-1H-pyrrolo[1,2-a]pyrazin-4-one hydrobromide) as an off-white solid (1.00 g, 63%): LCMS (ESI) calculated for C₁₃H₁₄Cl₂N₂O₂ [M+H]⁺ 301, 303 (3:2), found 301, 303 (3:2); ¹H NMR (400 MHz, CD₃OD) δ 7.29 (d, J=8.8 Hz, 1H), 6.79 (d, J=8.8 Hz, 1H), 4.44-4.28 (m, 1H), 4.28-4.18 (m, 1H), 4.18-4.05 (m, 1H), 3.98-3.84 (m, 3H), 3.71-3.55 (m, 1H), 3.19 (t, J=11.9 Hz, 1H), 2.49 (q, J=11.5 Hz, 1H), 2.37-2.24 (m, 1H).

### Example 8. Intermediate 9 (tert-butyl (2S,4R)-4-(2,3-dichloro-6-methoxyphenyl)-2-(hydroxymethyl)piperidine-1-carboxylate) and Intermediate 10 (tert-butyl (2S,4R)-4-(2,3-dichloro-6-methoxyphenyl)-2-formylpiperidine-1-carboxylate)

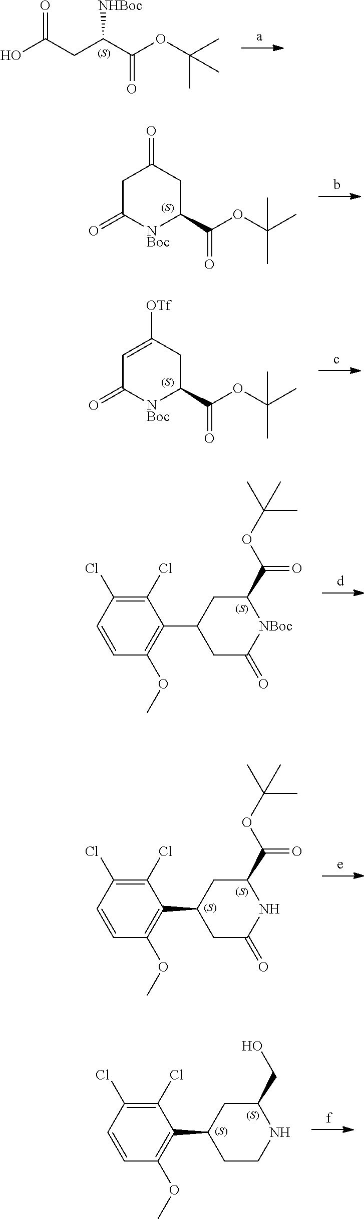

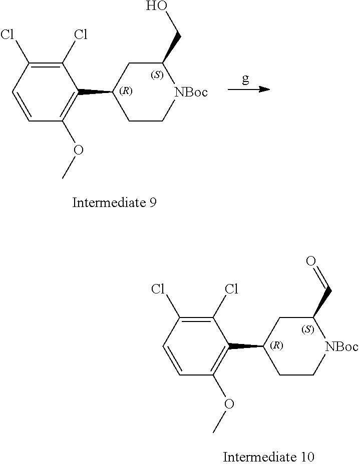

Step a:

To a stirred solution of (3S′)-4-(tert-butoxy)-3-[(tert-butoxycarbonyl)amino]-4-oxobutanoic acid (120 g, 415 mmol) in DCM (1.50 L) was added EDCI (120 g, 622 mmol), DMAP (76.0 g, 622 mmol) and 2,2-dimethyl-1,3-dioxane-4,6-dione (Meldrum's acid) (60.0 g, 414 mmol) at −8 TC. The resulting mixture was stirred at −8° C. for 3 h under nitrogen atmosphere. The resulting mixture was washed with saturated aq. KHSO₄ (2×1 L) and brine (2×1 L). The organic layer was dried over anhydrous Na₂SO₄. After filtration, the filtrate was concentrated under reduced pressure. The residue was dissolved in EA (3.70 L) to afford a 0.1 M solution, which was refluxed for 16 h. After being allowed to cool to room temperature, the mixture was washed with saturated aq. KHSO₄ (2×1 L) and brine (2×1 L). The organic layer was dried over anhydrous Na₂SO₄. After filtration, the filtrate was concentrated under reduced pressure to afford 1,2-di-tert-butyl (2S)-4,6-dioxopiperidine-1,2-dicarboxylate as an off-white solid (123 g, 94%): LCMS (ESI) calc'd for C₁₅H₂₃NO₆ [M+H]⁺: 314, found 314; ¹H NMR (400 MHz, CDCl₃) δ 5.12-5.03 (m, 1H), 3.63-3.32 (m, 2H), 3.10-3.01 (m, 1H), 2.89-2.79 (m, 1H), 1.58 (s, 9H), 1.49 (s, 9H).

Step b:

To a solution of 1,2-di-tert-butyl (2S)-4,6-dioxopiperidine-1,2-dicarboxylate (50.0 g, 160 mmol) in DCM (500 mL) was added DIEA (83 mL, 645 mmol) dropwise at 0° C. The resulting reaction was stirred for 10 min at 0° C., and then trifluoromethylsulfonic anhydride (54.0 g, 191 mmol) was added dropwise at 0° C. Then the reaction was allowed to warm to room temperature and stirred for an additional 2 h. The reaction was quenched with saturated aq. NaHCO₃ (100 mL) at 10° C. The aqueous phase was extracted with DCM (3×100 mL). The combined organic phases were washed with brine (2×100 mL) and dried over anhydrous Na₂SO₄. After filtration, the filtrate was concentrated under reduced pressure. The residue was purified by silica gel column chromatography, eluting with PE/EA (5/1) to afford 1,2-di-tert-butyl (2S)-6-oxo-4-(trifluoromethanesulfonyloxy)-2,3-dihydropyridine-1,2-dicarboxylate as a yellow solid (39.0 g, 55%): LCMS (ESI) calc'd for C₁₆H₂₂F₃NO₈S [M+H]⁺: 446 found 346 [M+H−100]⁺; ¹H NMR (300 MHz, CDCl₃) δ 6.03 (d, J=2.2 Hz, 1H), 5.01 (dd, J=6.3, 2.6 Hz, 1H), 3.27-2.98 (m, 2H), 1.57 (s, 9H), 1.47 (s, 9H).

Step c:

To a stirred mixture of 1,2-di-tert-butyl (2S)-6-oxo-4-(trifluoromethanesulfonyloxy)-2,3-dihydropyridine-1,2-dicarboxylate (39.0 g, 78.8 mmol), 2,3-dichloro-6-methoxyphenylboronic acid (20.0 g, 81.5 mmol) and Na₂CO₃ (17.0 g, 163 mmol) in dioxane (400 mL) and H₂O (100 mL) was added Pd(dppf)Cl₂.CH₂Cl₂ (2.66 g, 3.26 mmol) at room temperature under nitrogen atmosphere. The suspension was degassed under vacuum and purged with nitrogen atmosphere three times. The reaction was then stirred at 80° C. for 2 h under nitrogen atmosphere. The reaction mixture was concentrated under reduced pressure. The residue was diluted in EA (500 mL) and washed with brine (2×500 mL). The organic phase was dried over anhydrous Na₂SO₄. After filtration, the filtrate was concentrated under reduced pressure. The residue was purified by silica gel column chromatography, eluting with PE/EA (2/1) to afford 1,2-di-tert-butyl (2S)-4-(2,3-dichloro-6-methoxyphenyl)-6-oxo-2,3-dihydropyridine-1,2-dicarboxylate as a light yellow liquid (31.0 g, 72%): LCMS (ESI) calc'd for C₂₂H₂₇Cl₂NO₆ [M+H]⁺: 472, 474 (3:2) found 372, 374 [M+H−100]⁺ (3:2); ¹H NMR (300 MHz, CDCl₃) δ 7.42 (d, J=8.9 Hz, 1H), 6.80 (d, J=9.0 Hz, 1H), 5.92 (d, J=2.7 Hz, 1H), 4.95 (dd, J=7.2, 1.8 Hz, 1H), 3.78 (s, 3H), 3.14 (d, J=17.6 Hz, 1H), 2.90 (d, J=18.2 Hz, 1H), 1.60 (s, 9H), 1.50 (s, 9H).

Step d:

To a stirred solution of 1,2-di-tert-butyl (2S)-4-(2,3-dichloro-6-methoxyphenyl)-6-oxo-2,3-dihydropyridine-1,2-dicarboxylate (31.0 g, 65.6 mmol) in EA (400 mL) and AcOH (100 mL) was added PtO₂ (6.26 g, 27.6 mmol) in portions at room temperature. The resulting mixture was stirred at room temperature for 16 h under hydrogen atmosphere (1.5 atm), filtered, and the filter cake was then washed with MeOH (3×50 mL). The filtrate was concentrated under reduced pressure. The residue was purified by silica gel column chromatography, eluting with PE/EA (2/1) to afford tert-butyl (2S,4R)-4-(2,3-dichloro-6-methoxyphenyl)-6-oxopiperidine-2-carboxylate as a light yellow liquid (20.8 g, 76%): LCMS (ESI) calc'd for C₁₇H₂₁Cl₂NO₄ [M+H]⁺: 374, 376 (3:2) found 374, 376 (3:2); ¹H NMR (300 MHz, CDCl₃) δ 7.36 (d, J=8.9 Hz, 1H), 6.79 (d, J=8.9 Hz, 1H), 4.12-3.92 (m, 2H), 3.85 (s, 3H), 3.03 (dd, J=17.7, 11.2 Hz, 1H), 2.57-2.34 (m, 2H), 2.28-2.09 (m, 1H), 1.86-1.63 (m, 1H), 1.51 (d, J=2.1 Hz, 9H).

Step e:

To a stirred solution of tert-butyl (2S,4R)-4-(2,3-dichloro-6-methoxyphenyl)-6-oxopiperidine-2-carboxylate (20.8 g, 50.0 mmol) in THE (200 mL) was added BH₃Me₂S (14.2 mL, 187 mmol, 10 M in Me₂S solution) at room temperature under nitrogen atmosphere. The reaction was stirred at 70° C. for 4 h. The reaction was quenched with MeOH (50 mL) at 0° C. The resulting mixture was concentrated under reduced pressure. The residue was dissolved in MeOH (100 mL) and HCl (6 N, 100 mL). The resulting solution was stirred at 70° C. for 1 h and then concentrated under reduced pressure to afford [(2S,4R)-4-(2,3-dichloro-6-methoxyphenyl)piperidin-2-yl]methanol as a light yellow liquid, which was used directly in the next step without further purification (20.0 g, crude): LCMS (ESI) calc'd for C₁₃H₁₇Cl₂NO₂ [M+H]⁺: 290, 292 (3:2) found 290, 292 (3:2).

Step f:

To a stirred solution of [(2S,4R)-4-(2,3-dichloro-6-methoxyphenyl)piperidin-2-yl]methanol (20.0 g, 68.9 mmol) and TEA (28.7 mL, 284 mmol) in DCM (200 mL) was added Boc₂O (17.7 mL, 81.1 mmol) at room temperature. The reaction was stirred at room temperature for 1 h and then diluted with water (100 mL). The aqueous solution was extracted with DCM (2×200 mL). The combined organic layers were washed with brine (2×100 mL) and dried over anhydrous Na₂SO₄. After filtration, the filtrate was concentrated under reduced pressure. The residue was purified by silica gel column chromatography, eluting with PE/EA (1/1) to afford Intermediate 9 (tert-butyl (2S,4R)-4-(2,3-dichloro-6-methoxyphenyl)-2-(hydroxymethyl)piperidine-1-carboxylate) as a light yellow liquid (13.0 g, 43%): LCMS (ESI) calc'd for C₁₈H₂₅Cl₂NO₄ [M+H]⁺: 390, 392 (3:2) found 334, 336 [M+H−56]⁺ (3:2); ¹H NMR (400 MHz, CDCl₃) δ 7.30 (d, J=9.4 Hz, 1H), 6.75 (d, J=8.9 Hz, 1H), 3.82 (s, 3H), 3.80-3.56 (m, 5H), 3.54-3.40 (m, 1H), 2.40-2.24 (m, 1H), 2.06-1.96 (m, 1H), 1.87-1.74 (m, 1H), 1.60-1.55 (m, 1H), 1.53 (s, 9H).

Step g:

To a stirred solution of tert-butyl (2S,4R)-4-(2,3-dichloro-6-methoxyphenyl)-2-(hydroxymethyl)piperidine-1-carboxylate (1.40 g, 3.58 mmol) in DCM (10 mL) was added Dess-Martin reagent (1.80 g, 4.31 mmol) at room temperature. The reaction was stirred at room temperature for 1 h. The resulting mixture was quenched with saturated aq. Na₂S₂O₄ (10 mL) and saturated aq. NaHCO₃ (30 mL). The solution was extracted with EA (2×50 mL). The combined organic layers were washed with brine (2×50 mL) and dried over anhydrous Na₂SO₄. After filtration, the filtrate was concentrated under reduced pressure to afford Intermediate 10 (tert-butyl (2S,4R)-4-(2,3-dichloro-6-methoxyphenyl)-2-formylpiperidine-1-carboxylate) as a yellow liquid (1.30 g, crude): LCMS (ESI) calc'd for C₁₈H₂₃Cl₂NO₄ [M+H−56]⁺: 332, 334 (3:2) found 332, 334 (3:2); ¹H NMR (400 MHz, CD₃OD) δ 9.52 (d, J=1.4 Hz, 1H), 7.39 (d, J=9.0 Hz, 1H), 6.97 (d, J=9.0 Hz, 1H), 4.02-3.90 (m, 1H), 3.90-3.64 (m, 5H), 3.26-3.10 (m, 1H), 2.45-2.22 (m, 2H), 1.93-1.57 (m, 2H), 1.51 (d, J=5.8 Hz, 9H).

### Example 9. Intermediate 11 ((8R,9aS)-8-(2,3-dichloro-6-methoxyphenyl)-octahydropyrido[1,2-a]pyrazin-4-one)

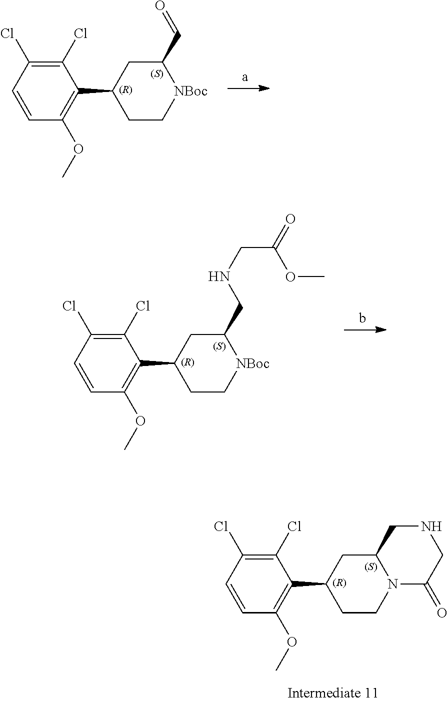

Step a:

To a stirred solution of tert-butyl (2S,4R)-4-(2,3-dichloro-6-methoxyphenyl)-2-formylpiperidine-1-carboxylate (1.30 g, 3.35 mmol) (Intermediate 10, Example 8) and methyl glycinate hydrochloride (0.640 g, 5.09 mmol) in DCM (10 mL) were added TEA (0.510 g, 5.04 mmol) and NaBH(OAc)₃ (1.42 g, 6.70 mmol) at room temperature. The reaction was stirred at room temperature for 16 h. The reaction was diluted with EA (20 mL) and water (20 mL). The aqueous solution was extracted with EA (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 35% ACN in water (plus 0.05% TFA) to afford tert-butyl (2S,4R)-4-(2,3-dichloro-6-methoxyphenyl)-2-[[(2-methoxy-2-oxoethyl)amino]methyl]piperidine-1-carboxylate trifluoroacetic acid salt as a colorless liquid (1.00 g, 52%): LCMS (ESI) calc'd for C₂₁H₃₀Cl₂N₂O₅ [M+H]⁺: 461, 463 (3:2) found 461, 463 (3:2); ¹H NMR (400 MHz, CD₃OD) δ 7.41 (d, J=8.8 Hz, 1H), 6.99 (d, J=8.9 Hz, 1H), 4.20 (s, 1H), 4.12-3.96 (m, 2H), 3.88 (d, J=1.2 Hz, 6H), 3.76-3.54 (m, 2H), 3.54-3.37 (m, 2H), 3.22-3.10 (m, 1H), 2.41 (d, J=13.2 Hz, 1H), 2.00-1.87 (m, 2H), 1.69 (d, J=13.3 Hz, 1H), 1.57 (s, 9H); ¹⁹F NMR (376 MHz, CD₃OD) δ −77.31 (s, 3H).

Step b:

To a stirred solution of tert-butyl (2S,4R)-4-(2,3-dichloro-6-methoxyphenyl)-2-[[(2-methoxy-2-oxoethyl)amino]methyl]piperidine-1-carboxylate trifluoroacetic acid (1.00 g, 1.74 mmol) in DCM (10 mL) was added TFA (4 mL) at room temperature. The reaction was stirred at room temperature for 1 h and then concentrated under reduced pressure. The residue was dissolved in EtOH (10 mL) and TEA (0.530 g, 5.24 mmol) was added. The reaction was stirred at 80° C. for 1 h and then diluted with EA (50 mL) and water (30 mL). The aqueous solution was extracted with EA (2×30 mL). The combined organic layers were washed with brine (2×30 mL) and dried over anhydrous Na₂SO₄. After filtration, the filtrate was concentrated under reduced pressure to afford Intermediate 11 ((8R,9aS)-8-(2,3-dichloro-6-methoxyphenyl)-octahydropyrido[1,2-a]pyrazin-4-one) as an off-white foam (0.550 g, crude): LCMS (ESI) calc'd for C₁₅H₁₈Cl₂N₂O₂ [M+H]⁺: 329, 331 (3:2) found 329, 331 (3:2). ¹H NMR (400 MHz, CD₃OD) δ 7.39 (d, J=9.0 Hz, 1H), 6.97 (d, J=9.0 Hz, 1H), 4.78 (ddd, J=13.3, 4.4, 2.2 Hz, 1H), 3.85 (s, 3H), 3.80-3.68 (m, 1H), 3.63-3.54 (m, 1H), 3.52 (d, J=2.0 Hz, 2H), 3.30 (d, J=5.2 Hz, 1H), 2.86 (dd, J=13.3, 8.4 Hz, 1H), 2.71 (td, J=13.2, 3.0 Hz, 1H), 2.41-2.23 (m, 2H), 1.72-1.62 (m, 2H).

### Example 10. Intermediate 12 ((8R,9aS)-8-(2,3-dichloro-6-hydroxyphenyl)-octahydropyrido[1,2-a]pyrazin-4-one)

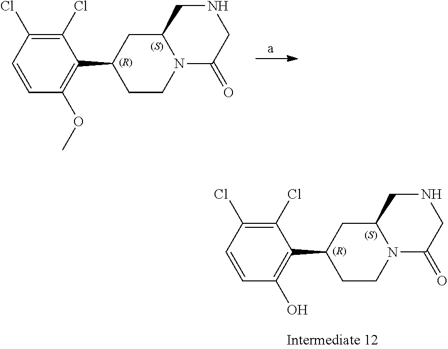

Step a:

To a stirred solution of (8R,9aS)-8-(2,3-dichloro-6-methoxyphenyl)-octahydropyrido[1,2-a]pyrazin-4-one (Intermediate 11, Example 9) (0.550 g, 1.67 mmol) in DCM (5 mL) was added BBr₃ (4.19 g, 16.7 mmol) at room temperature. The reaction was stirred at room temperature for 1 h. The reaction was quenched with MeOH (2 mL) and the resulting solution was concentrated under reduced pressure. The residue was dissolved in MeOH (5 mL) and basified with TEA to PH 8. After being concentrated under reduced pressure, the residue was purified by reverse phase chromatography, eluting with 36% ACN in water (plus 10 mM NH₄HCO₃) to afford the crude product. The fractions containing the desired product were combined and concentrated under reduced pressure to afford Intermediate 12 ((8R,9aS)-8-(2,3-dichloro-6-hydroxyphenyl)-octahydropyrido[1,2-a]pyrazin-4-one) as an off-white solid (0.250 g, 47%): LCMS (ESI) calc'd for C₁₄H₁₆Cl₂N₂O₂ [M+H]⁺: 315, 317 (3:2) found 315, 317 (3:2); ¹H NMR (400 MHz, CD₃OD) δ 7.20 (d, J=8.8 Hz, 1H), 6.72 (d, J=8.8 Hz, 1H), 4.81-4.73 (m, 1H), 3.76-3.63 (m, 1H), 3.60-3.49 (m, 1H), 3.44 (s, 2H), 3.24 (dd, J=13.4, 5.1 Hz, 1H), 2.81-2.61 (m, 2H), 2.56-2.31 (m, 2H), 1.70-1.59 (m, 2H).

### Example 11. Intermediate 13 ((7R,8aS)-7-[2,3-dichloro-6-(prop-2-en-1-yloxy)phenyl]-hexahydro-1H-pyrrolo[1,2-a]pyrazin-4-one)

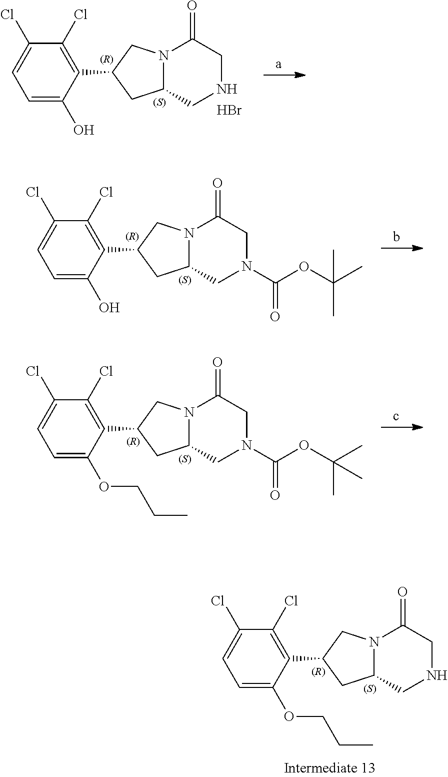

Step a:

To a stirred solution of (7R,8aS)-7-(2,3-dichloro-6-hydroxyphenyl)-hexahydro-1H-pyrrolo[1,2-a]pyrazin-4-one hydrobromide (2.00 g, 5.24 mmol) and TEA (1.59 g, 15.7 mmol) in DCM (20 mL) was added Boc₂O (1.14 g, 5.24 mmol) at room temperature. The resulting mixture was stirred at room temperature for 1 h, diluted with water (50 mL) and extracted with EA (3×40 mL). The combined organic layers were washed with brine (3×20 mL) and dried over anhydrous Na₂SO₄. After filtration, the filtrate was concentrated under reduced pressure to afford tert-butyl (7R,8aS)-7-(2,3-dichloro-6-hydroxyphenyl)-4-oxo-hexahydropyrrolo[1,2-a]pyrazine-2-carboxylate as a light yellow solid, which was used directly in the next step without further purification (2.10 g, crude): LCMS (ESI) calc'd for C₁₈H₂₂Cl₂N₂O₄ [M+H]⁺: 401, 403 (3:2), found 401, 403 (3:2).

Step b:

To a stirred solution of tert-butyl (7R,8aS)-7-(2,3-dichloro-6-hydroxyphenyl)-4-oxo-hexahydropyrrolo[1,2-a]pyrazine-2-carboxylate (2.10 g, 5.23 mmol) and K₂CO₃ (1.45 g, 10.5 mmol) in DMF (40 mL) was added allyl bromide (0.760 g, 6.28 mmol) at room temperature. The resulting mixture was stirred at room temperature for 3 h, diluted with water (100 mL) and then extracted with EA (3×50 mL). The combined organic layers were washed with brine (5×30 mL) and dried over anhydrous Na₂SO₄. After filtration, the filtrate was concentrated under reduced pressure to afford tert-butyl (7R,8aS)-7-[2,3-dichloro-6-(prop-2-en-1-yloxy)phenyl]-4-oxo-hexahydropyrrolo[1,2-a]pyrazine-2-carboxylate as a light yellow solid, which was used directly in the next step without further purification (2.10 g, crude): LCMS (ESI) calc'd for C₂₁H₂₆Cl₂N₂O₄ [M+H]⁺: 441, 443 (3:2), found 441, 443 (3:2).

Step c:

To a stirred solution of tert-butyl (7R,8aS)-7-[2,3-dichloro-6-(prop-2-en-1-yloxy)phenyl]-4-oxo-hexahydropyrrolo[1,2-a]pyrazine-2-carboxylate (2.00 g, 4.53 mmol) in DCM (20 mL) was added TFA (10 mL) at room temperature. The resulting solution was stirred at room temperature for 1 h and concentrated under reduced pressure. The residue was purified by reverse phase flash chromatography, eluting with 60% ACN in water (plus 10 mM NH₄HCO₃) to afford Intermediate 13 ((7R,8aS)-7-[2,3-dichloro-6-(prop-2-en-1-yloxy)phenyl]-hexahydro-1H-pyrrolo[1,2-a]pyrazin-4-one) as a light yellow liquid (1.50 g, 66% over three steps): LCMS (ESI) calc'd for C₁₆H₁₈Cl₂N₂O₂ [M+H]⁺: 341, 343 (3:2), found 341, 343 (3:2); ¹H NMR (400 MHz, CDCl₃) δ 7.32 (d, J=8.9 Hz, 1H), 6.76 (d, J=9.0 Hz, 1H), 6.09-5.95 (m, 1H), 5.43-5.30 (m, 2H), 4.59-4.44 (m, 2H), 4.30-4.14 (m, 2H), 3.83-3.72 (m, 1H), 3.67-3.53 (m, 2H), 3.50-3.38 (m, 2H), 2.64 (dd, J=12.7, 10.2 Hz, 1H), 2.24-2.06 (m, 2H).

### Example 12. Intermediate 14 (8-(2,3-dichloro-6-hydroxyphenyl)-3-(hydroxymethyl)-hexahydro-1H-pyrido[2,1-c][1,4]oxazin-4-one)

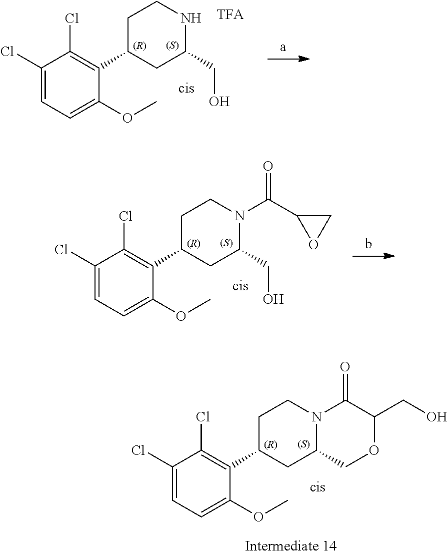

Step a:

To a stirred solution of glycidic acid (0.668 g, 7.58 mmol) and HATU (3.17 g, 8.34 mmol) in DMF (20.0 mL) were added [(2S,4R)-4-(2,3-dichloro-6-methoxyphenyl)piperidin-2-yl]methanol (Intermediate 9, Example 8) (2.20 g, 7.58 mmol) and TEA (2.30 g, 22.7 mmol) at room temperature. The resulting reaction mixture was stirred at room temperature for 1 h diluted with water (100 mL) and extracted with EA (2×80 mL). The combined organic layers were washed with brine (2×80 mL) and dried over anhydrous Na₂SO₄. After filtration, the filtrate was concentrated under reduced pressure. The reaction was purified by reverse phase chromatography, eluting with 33% ACN in water (plus 10 mM NH₄HCO₃) to afford [(2S,4R)-4-(2,3-dichloro-6-methoxyphenyl)-1-(oxirane-2-carbonyl)piperidin-2-yl]methanol as an off-white semisolid (1.10 g, 40%): LCMS (ESI) calc'd for C₁₆H₁₉Cl₂NO₄ [M+1]⁺ 360, 362 (3:2) found 360, 362 (3:2); ¹H NMR (400 MHz, CD₃OD) δ 7.39 (d, J=8.9 Hz, 1H), 6.99 (d, J=9.0 Hz, 1H), 4.49-3.93 (m, 3H), 3.85 (s, 3H), 3.83-3.57 (m, 3H), 3.08-2.94 (m, 1H), 2.94-2.79 (m, 1H), 2.81-2.56 (m, 1H), 2.18-1.89 (m, 2H), 1.85-1.54 (m, 1H), 1.39-1.28 (m, 1H).

Step b:

To a stirred solution of [(2S,4R)-4-(2,3-dichloro-6-methoxyphenyl)-1-(oxirane-2-carbonyl)piperidin-2-yl]methanol (1.10 g, 3.05 mmol) in THE (10.0 mL) was added t-BuOK (0.516 g, 4.61 mmol) at 0° C. under nitrogen atmosphere. The reaction was stirred at 0° C. for 1 h. The resulting mixture was quenched with water (100 mL) and extracted with EA (3×30 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 0.05% TFA) to afford Intermediate 14 ((8R,9aS)-8-(2,3-dichloro-6-methoxyphenyl)-3-(hydroxymethyl)-hexahydro-1H-pyrido[2,1-c][1,4]oxazin-4-one) as a yellow liquid (0.450 g, 50%): LCMS (ESI) calc'd for C₁₆H₁₉Cl₂NO₄ [M+1]⁺ 360, 362 (3:2) found 360, 362 (3:2); ¹H NMR (400 MHz, CD₃OD) δ 7.38 (d, J=9.0, 1H), 6.96 (d, J=9.0, 1H), 4.78-4.65 (m, 1H), 4.20-4.10 (m, 1H), 4.05-3.94 (m, 2H), 3.94-3.87 (m, 2H), 3.84 (d, J=6.7 Hz, 3H), 3.77-3.68 (m, 1H), 3.53-3.44 (m, 1H), 2.81-2.68 (m, 2H), 2.40-2.28 (m, 1H), 1.80-1.52 (m, 2H).

### Example 13. Intermediate 15 ((2R,8aS)-2-(2,3-dichloro-6-methoxyphenyl)-hexahydroindolizine-5,7-dione)

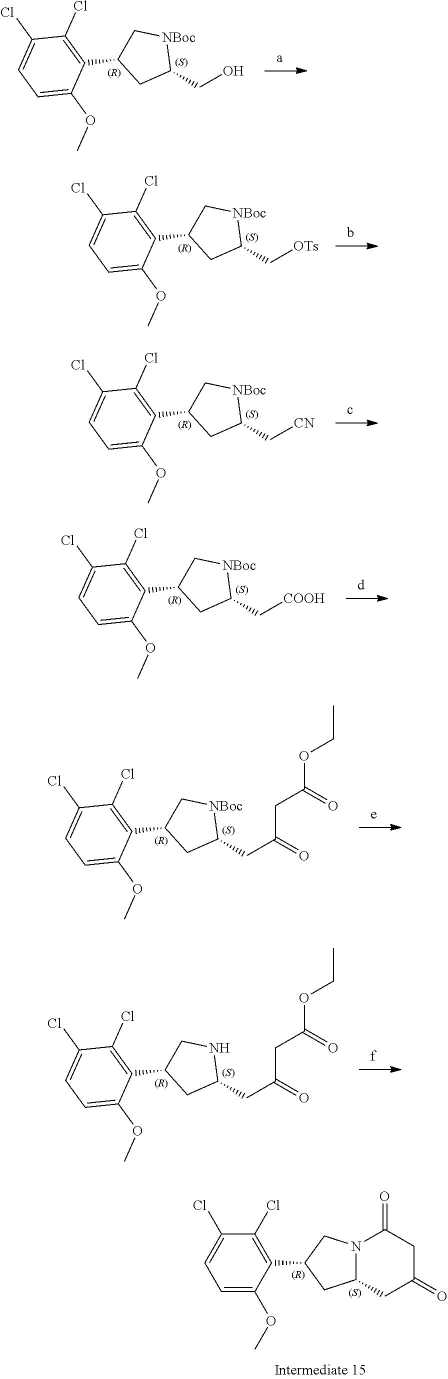

Step a:

To a stirred solution of tert-butyl (2S,4R)-4-(2,3-dichloro-6-methoxyphenyl)-2-(hydroxymethyl)pyrrolidine-1-carboxylate (Example 7, step b) (40.0 g, 95.7 mmol), TsCl (21.9 g, 115 mmol) and DMAP (3.51 g, 28.7 mmol) in DCM (400 mL) was added TEA (26.6 g, 263 mmol) dropwise at room temperature. The resulting mixture was stirred at room temperature for 4 h under nitrogen and then diluted with water (300 mL). The aqueous solution was extracted with DCM (3×200 mL). The combined organic layers were washed with brine (2×100 mL) and dried over anhydrous Na₂SO₄. After filtration, the filtrate was concentrated under reduced pressure. The residue was purified by silica gel column chromatography, eluting with PE/EA (3/1) to afford tert-butyl (2S,4R)-4-(2,3-dichloro-6-methoxyphenyl)-2-[[(4-methylbenzenesulfonyl)oxy]methyl]pyrrolidine-1-carboxylate as an off-white solid (42.5 g, 75%): LCMS (ESI) calc'd for C₂₄H₂₉Cl₂NO₆S [M+H−100]⁺: 430, 432 (3:2) found 430, 432 (3:2); ¹H NMR (300 MHz, CD₃OD) δ 7.82 (d, J=7.9 Hz, 2H), 7.48 (d, J=7.9 Hz, 2H), 7.42 (d, J=9.0 Hz, 1H), 6.99 (d, J=9.0 Hz, 1H), 4.47-4.28 (m, 1H), 4.19-3.97 (m, 3H), 3.89 (s, 3H), 3.76-3.56 (m, 2H), 2.69 (q, J=11.1 Hz, 1H), 2.47 (s, 3H), 2.26-2.07 (m, 1H), 1.42 (s, 9H).

Step b:

To a stirred solution of tert-butyl (2S,4R)-4-(2,3-dichloro-6-methoxyphenyl)-2-[[(4-methylbenzenesulfonyl)oxy]methyl]pyrrolidine-1-carboxylate (12.0 g, 22.6 mmol) in DMSO (20 mL) was added KCN (2.95 g, 45.3 mmol) at room temperature. The resulting solution was stirred at 80° C. for 1 h, diluted with saturated aq. NaHCO₃ (100 mL) and then extracted with EA (3×100 mL). The combined organic layers were washed with brine (3×100 mL) and dried over anhydrous Na₂SO₄. After filtration, the filtrate was concentrated under reduced pressure. The residue was purified by silica gel chromatography, eluting with PE/EA (2/1) to afford tert-butyl (2S,4R)-2-(cyanomethyl)-4-(2,3-dichloro-6-methoxyphenyl)pyrrolidine-1-carboxylate as an off-white solid (3.50 g, 40%): LCMS (ESI) calc'd for C₁₈H₂₂Cl₂N₂O₃ [M+H]⁺: 385, 387 (3:2) found 385, 387 (3:2); ¹H NMR (400 MHz, CD₃OD) δ 7.44 (d, J=9.0 Hz, 1H), 7.02 (d, J=9.0 Hz, 1H), 4.17-4.04 (m, 2H), 3.95-3.90 (m, 4H), 3.69-3.64 (m, 1H), 3.21-3.19 (m, 1H), 2.88-2.67 (m, 2H), 2.35-2.30 (m, 1H), 1.53 (s, 9H).

Step c:

To a stirred solution of tert-butyl (2S,4R)-2-(cyanomethyl)-4-(2,3-dichloro-6-methoxyphenyl)pyrrolidine-1-carboxylate (9.30 g, 24.1 mmol) in conc. HCl (20 mL) was added AcOH (4 mL) at room temperature. The reaction was stirred at 100° C. for 1 h. After being allowed to cool to room temperature, the resulting mixture was concentrated under reduced pressure. DCM (20 mL), TEA (12.2 g, 121 mmol) and Boc₂O (5.79 g, 26.6 mmol) were then added sequentially to the crude product. The reaction was stirred at room temperature for 1 h and concentrated under reduced pressure. The residue was purified by reverse phase chromatography, eluting with 65% ACN in water (plus 0.1% FA) to afford [(2S,4R)-1-(tert-butoxycarbonyl)-4-(2,3-dichloro-6-methoxyphenyl)pyrrolidin-2-yl]acetic acid as an off-white solid (9.00 g, 92%): LCMS (ESI) calc'd C₁₈H₂₃Cl₂NO₅ for [M+H]⁺: 404, 406 (3:2) found 404, 406 (3:2); ¹H NMR (300 MHz, CD₃OD) δ 7.42 (d, J=9.0 Hz, 1H), 6.99 (d, J=9.0 Hz, 1H), 4.28-3.99 (m, 2H), 3.89 (s, 3H), 3.84-3.80 (m, 1H), 3.70-3.58 (m, 1H), 3.17-2.87 (m, 1H), 2.63-2.30 (m, 3H), 1.51 (s, 9H).

Step d:

To a stirred solution of [(2S,4R)-1-(tert-butoxycarbonyl)-4-(2,3-dichloro-6-methoxyphenyl)pyrrolidin-2-yl]acetic acid (9.00 g, 22.3 mmol) and 2,2-dimethyl-1,3-dioxane-4,6-dione (Meldrum's acid) (4.81 g, 33.4 mmol) in DCM (50.0 mL) were added DMAP (4.08 g, 33.4 mmol) and EDCI (6.40 g, 33.5 mmol) at room temperature. The reaction was stirred at room temperature for 3 h. The resulting solution was diluted with DCM (100 mL), washed with aq. HCl (1 M, 2×100 mL) and brine (3×100 mL), and dried over anhydrous Na₂SO₄. After filtration, the filtrate was concentrated under reduced pressure. The residue was dissolved in EtOH (30 mL) and stirred at 90° C. for 1 h. The resulting solution was diluted with water (100 mL) and extracted with EA (3×80 mL). The combined organic layers were washed with brine (3×80 mL) and dried over anhydrous Na₂SO₄. After filtration, the filtrate was concentrated under reduced pressure. The residue was purified by silica gel column chromatography, eluting with PE/EA (2/1) to afford tert-butyl (2S,4R)-4-(2,3-dichloro-6-methoxyphenyl)-2-(4-ethoxy-2,4-dioxobutyl)pyrrolidine-1-carboxylate as a light yellow liquid (9.00 g, 85%): LCMS (ESI) calc'd C₂₂H₂₉Cl₂NO₆ for [M+H]⁺: 474, 476 (3:2) found 474, 476 (3:2); ¹H NMR (300 MHz, CDCl₃) δ 7.33 (d, J=8.9 Hz, 1H), 6.76 (d, J=9.0 Hz, 1H), 4.29-3.99 (m, 4H), 4.17-4.01 (m, 1H), 3.85 (s, 3H), 3.81-3.68 (m, 1H), 3.55-3.36 (m, 3H), 2.85-2.80 (m, 1H), 2.49-2.25 (m, 2H), 1.50 (s, 9H), 1.29 (t, J=7.2 Hz, 3H).

Step e:

To a stirred solution of tert-butyl (2S,4R)-4-(2,3-dichloro-6-methoxyphenyl)-2-(4-ethoxy-2,4-dioxobutyl)pyrrolidine-1-carboxylate (9.00 g, 19.0 mmol) in DCM (40 mL) was added TFA (10 mL) at room temperature. The reaction was stirred at room temperature for 1 h. The resulting solution was concentrated under reduced pressure to afford ethyl 4-[(2S,4R)-4-(2,3-dichloro-6-methoxyphenyl)pyrrolidin-2-yl]-3-oxobutanoate as a yellow liquid (9.00 g, crude), which was used directly in the next step without further purification: LCMS (ESI) calc'd C₁₇H₂₁Cl₂NO₄ for [M+H]⁺: 374, 376 (3:2) found 374, 376 (3:2).

Step f:

To a stirred solution of ethyl 4-[(2S,4R)-4-(2,3-dichloro-6-methoxyphenyl)pyrrolidin-2-yl]-3-oxobutanoate (9.00 g, 24.1 mmol) in MeOH (50 mL) was added K₂CO₃ (16.7 g, 120 mmol) at room temperature. The resulting mixture was stirred at room temperature for 1 h and then neutralized with aq. HCl (1 M) to pH 7 and extracted with EA (3×100 mL). The combined organic layers were washed with brine (3×80 mL) and dried over anhydrous Na₂SO₄. After filtration, the filtrate was concentrated under reduced pressure. The residue was purified by silica gel column chromatography, eluting with EA to afford Intermediate 15 ((2R,8aS)-2-(2,3-dichloro-6-methoxyphenyl)-hexahydroindolizine-5,7-dione) as a light yellow solid (5.00 g, 80% overall two steps): LCMS (ESI) calc'd C₁₅H₁₅Cl₂NO₃ for [M+H]⁺: 328, 330 (3:2) found 328, 330 (3:2); ¹H NMR (300 MHz, CDCl₃) δ 7.38 (d, J=8.9, 1H), 6.81 (d, J=9.0, 1H), 4.40-4.12 (m, 2H), 4.12-3.98 (m, 1H), 3.85 (s, 3H), 3.83-3.71 (m, 1H), 3.34 (s, 2H), 2.96-2.76 (m, 1H), 2.74-2.29 (m, 3H).

### Example 14. Intermediate 16 ((2R,8aS)-2-(2,3-dichloro-6-methoxyphenyl)-2,3,6,8a-tetrahydro-1H-indolizin-5-one)

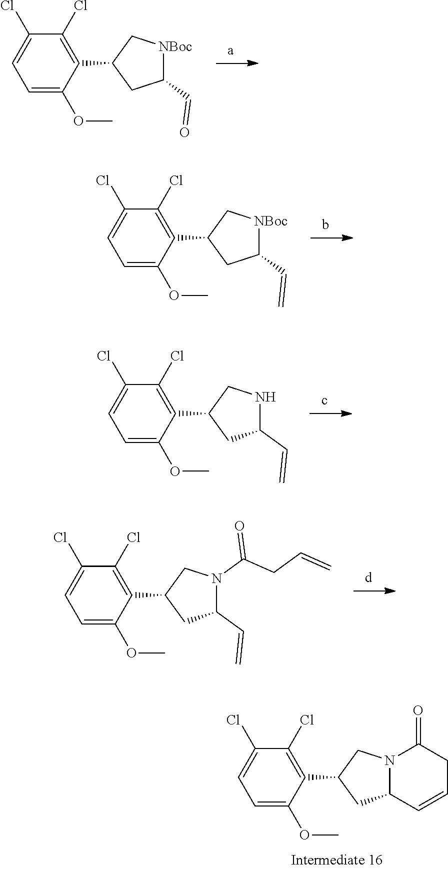 [SMILES: COc1ccc(Cl)c(Cl)c1[C@H]1C[C@@H](C=O)N(C(=O)OC(C)(C)C)C1 ; C=C[C@@H]1C[C@H](c2c(OC)ccc(Cl)c2Cl)CN1C(=O)OC(C)(C)C ; C=C[C@@H]1C[C@H](c2c(OC)ccc(Cl)c2Cl)CN1 ; C=CCC(=O)N1C[C@@H](c2c(OC)ccc(Cl)c2Cl)C[C@H]1C=C ; COc1ccc(Cl)c(Cl)c1[C@H]1C[C@H]2C=CCC(=O)N2C1]

Step a:

To a solution of methyltriphenylphosphanium bromide (48.7 g, 136 mmol) in THE (400 mL) was added t-BuOK (136 mL, 136 mmol, 1 M in THF) dropwise for 30 min at −10° C. under nitrogen atmosphere. Then tert-butyl (2S,4R)-4-(2,3-dichloro-6-methoxyphenyl)-2-formylpyrrolidine-1-carboxylate (Example 7, step c) (17.0 g, 45.4 mmol) in THE (50 mL) was added dropwise into the mixture at −10° C. The resulting mixture was stirred at room temperature for 2 h under nitrogen, quenched with saturated aq. NH₄Cl (200 mL) at 0° C. and extracted with EA (3×300 mL). The combined organic layers were washed with brine (3×50 mL) and dried over anhydrous Na₂SO₄. After filtration, the filtrate was concentrated under reduced pressure. The residue was purified by silica gel column chromatography, eluting with PE/EA (5/1) to afford tert-butyl (2S,4R)-4-(2,3-dichloro-6-methoxyphenyl)-2-ethenylpyrrolidine-1-carboxylate as a colorless liquid (8.50 g, 43%): LCMS (ESI) calc'd for C₁₈H₂₃Cl₂NO₃ [M+H−56]⁺: 316, 318 (3:2), found 316, 318 (3:2); ¹H NMR (400 MHz, CD₃OD) δ 7.42 (d, J=9.0 Hz, 1H), 7.00 (d, J=9.0 Hz, 1H), 5.95-5.77 (m, 1H), 5.25-5.05 (m, 2H), 4.40-4.27 (m, 1H), 4.18-4.02 (m, 1H), 3.88 (s, 3H), 3.83-3.80 (m, 1H), 3.70-3.62 (m, 1H), 2.52-2.39 (m, 1H), 2.32-2.21 (m, 1H), 1.47 (s, 9H).

Step b:

To a stirred solution of tert-butyl (2S,4R)-4-(2,3-dichloro-6-methoxyphenyl)-2-ethenylpyrrolidine-1-carboxylate (3.60 g, 9.67 mmol) in DCM (36 mL) was added TFA (9 mL) at room temperature. The resulting mixture was stirred at room temperature for 1 h and concentrated under reduced pressure to afford (2S,4R)-4-(2,3-dichloro-6-methoxyphenyl)-2-ethenylpyrrolidine as a yellow liquid (3.60 g, crude), which was used directly in the next step without purification: LCMS (ESI) calc'd for C₁₃H₁₅Cl₂NO [M+H]⁺: 272, 274 (3:2), found 272, 274 (3:2).

Step c:

To a stirred solution of (2S,4R)-4-(2,3-dichloro-6-methoxyphenyl)-2-ethenylpyrrolidine (3.60 g, 13.2 mmol) and TEA (4.02 g, 39.7 mmol) in DMF (30 mL) were added 3-butenoic acid (1.37 g, 15.9 mmol) and diethyl cyanophosphonate (3.12 g, 17.2 mmol) at room temperature. The resulting mixture was stirred at room temperature for 16 h, quenched with water (100 mL) at room temperature and extracted with EA (3×60 mL). The combined organic layers were washed with brine (5×30 mL) and dried over anhydrous Na₂SO₄. After filtration, the filtrate was concentrated under reduced pressure. The residue was purified by silica gel column chromatography, eluting with PE/EA (4/1) to afford 1-[(2S,4R)-4-(2,3-dichloro-6-methoxyphenyl)-2-ethenylpyrrolidin-1-yl]but-3-en-1-one as a light yellow liquid (2.60 g, 79% over two steps): LCMS (ESI) calc'd for C₁₇H₁₉Cl₂NO₂ [M+H]⁺: 340, 342 (3:2), found 340, 342 (3:2); ¹H NMR (300 MHz, CDCl₃) δ 7.35 (d, J=8.8 Hz, 1H), 6.78 (d, J=8.9 Hz, 1H), 6.11-5.82 (m, 2H), 5.36-5.07 (m, 4H), 4.73-4.34 (m, 1H), 4.18-3.94 (m, 2H), 3.92-3.59 (m, 4H), 3.16 (d, J=6.6 Hz, 2H), 2.59-2.23 (m, 2H).

Step d:

To a stirred mixture of 1-[(2S,4R)-4-(2,3-dichloro-6-methoxyphenyl)-2-ethenylpyrrolidin-1-yl]but-3-en-1-one (2.60 g, 7.64 mmol) in DCM (26 mL) was added Grubbs 2^{nd} generation catalyst (0.260 g, 0.30 mmol) at room temperature. The resulting mixture was stirred at 40° C. for 16 h and then concentrated under reduced pressure. The residue was purified by silica gel column chromatography, eluting with EA to afford Intermediate 16 ((2R,8aS)-2-(2,3-dichloro-6-methoxyphenyl)-2,3,6,8a-tetrahydro-1H-indolizin-5-one) as a brown solid (2.20 g, 74%): LCMS (ESI) calc'd for C₁₅H₁₅Cl₂NO₂ [M+H]⁺: 312, 314 (3:2), found 312, 314 (3:2); ¹H NMR (400 MHz, CDCl₃) δ 7.33 (d, J=8.9 Hz, 1H), 6.75 (d, J=8.9 Hz, 1H), 5.94-5.82 (m, 2H), 4.33-4.20 (m, 3H), 3.79 (s, 3H), 3.55-3.50 (m, 1H), 3.07-2.98 (m, 2H), 2.27-2.13 (m, 2H).

### Example 15. Intermediate 17 ((2R,8aR)-2-(2,3-dichloro-6-methoxyphenyl)-2,3,8,8a-tetrahydro-1H-indolizin-5-one)

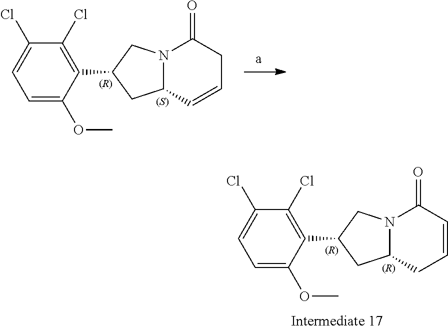

Step a:

To a stirred mixture of (2R,8aS)-2-(2,3-dichloro-6-methoxyphenyl)-2,3,6,8a-tetrahydro-1H-indolizin-5-one (Intermediate 16, Example 14) (2.20 g, 7.05 mmol) in toluene (15 mL) was added DBU (10 mL, 66.9 mmol) at room temperature. The resulting mixture was stirred at 90° C. for 16 h, diluted with water (100 mL) and extracted with EA (3×40 mL). The combined organic layers were washed with brine (3×20 mL) and dried over anhydrous Na₂SO₄. After filtration, the filtrate was concentrated under reduced pressure. The residue was purified by silica gel column chromatography, eluting with EA to afford Intermediate 17 ((2R,8aR)-2-(2,3-dichloro-6-methoxyphenyl)-2,3,8,8a-tetrahydro-1H-indolizin-5-one) as an off-white solid (1.50 g, 61%): LCMS (ESI) calc'd for C₁₅H₁₅Cl₂NO₂ [M+H]⁺: 312, 314 (3:2), found 312, 314 (3:2); ¹H NMR (400 MHz, CDCl₃) δ 7.35 (d, J=8.9 Hz, 1H), 6.78 (d, J=9.0 Hz, 1H), 6.61-6.55 (m, 1H), 6.06-6.02 (m, 1H), 4.28-4.11 (m, 1H), 4.01-3.94 (m, 1H), 3.94-3.87 (m, 1H), 3.84 (s, 3H), 3.80-3.71 (m, 1H), 2.58-2.49 (m, 1H), 2.45-2.40 (m, 1H), 2.34-2.20 (m, 2H).

### Example 16. Intermediate 18 ((6R,7aR)-6-(2,3-dichloro-6-methoxyphenyl)-hexahydropyrrolizin-3-one)

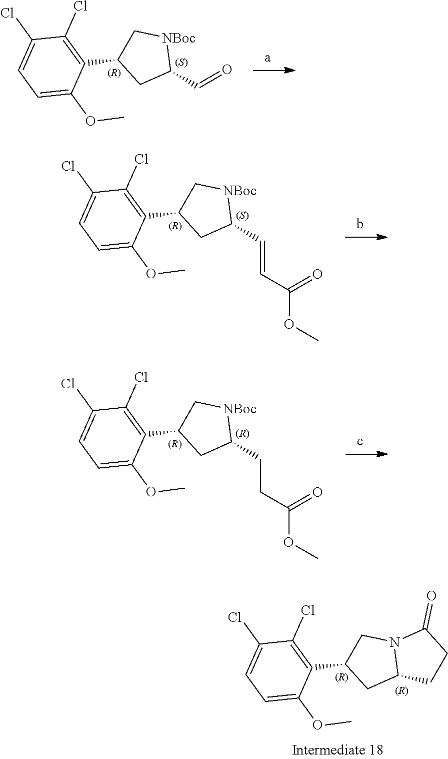

Step a:

To a solution of tert-butyl (2S,4R)-4-(2,3-dichloro-6-methoxyphenyl)-2-formylpyrrolidine-1-carboxylate (Example 7, step c) (2.00 g, 5.34 mmol) in DCM (30 mL) was added methyl 2-(triphenyl-λ5-phosphanylidene)acetate (1.79 g, 5.34 mmol) at room temperature. The reaction was stirred at room temperature for 16 h under nitrogen atmosphere and then concentrated under reduced pressure. The residue was purified by silica gel column chromatography, eluting with PE/EA (2/1) to afford tert-butyl (2S,4R)-4-(2,3-dichloro-6-methoxyphenyl)-2-(3-methoxy-3-oxoprop-1-en-1-yl)pyrrolidine-1-carboxylate as an off-white solid (1.65 g, 72%): LCMS (ESI) calc'd for C₂₀H₂₅Cl₂NO₅ [M+Na]⁺: 452, 454 (3:2), found 452, 454 (3:2); ¹H NMR (300 MHz, CDCl₃) δ 7.35 (d, J=8.9 Hz, 1H), 6.99-6.95 (m, 1H), 6.77 (d, J=9.0 Hz, 1H), 6.00-5.92 (m, 1H), 4.68-4.34 (m, 1H), 4.26-4.03 (m, 1H), 3.82 (s, 6H), 3.80-3.70 (m, 2H), 2.45-2.16 (m, 1H), 2.32-2.29 (m, 1H), 1.50 (s, 9H).

Step b:

To a stirred solution of tert-butyl (2S,4R)-4-(2,3-dichloro-6-methoxyphenyl)-2-(3-methoxy-3-oxoprop-1-en-1-yl)pyrrolidine-1-carboxylate (0.450 g, 1.05 mmol) in MeOH (6 mL) was added PtO₂ (50.0 mg, 0.220 mmol). The mixture was degassed under reduced pressure and purged with hydrogen three times. The mixture was stirred under hydrogen atmosphere (1.5 atm) at room temperature for 4 h. Then the mixture was filtered and concentrated under reduced pressure to afford tert-butyl (2R,4R)-4-(2,3-dichloro-6-methoxyphenyl)-2-(3-methoxy-3-oxopropyl)pyrrolidine-1-carboxylate as a colorless liquid (0.450 g, crude), which was used directly in the next step without purification: LCMS (ESI) calc'd for C₂₀H₂₇Cl₂NO₅ [M+H]⁺: 432, 434 (3:2), found 432, 434 (3:2); ¹H NMR (300 MHz, CDCl₃) δ 7.33 (d, J=8.9 Hz, 1H), 6.77 (d, J=8.9 Hz, 1H), 4.11-3.91 (m, 2H), 3.86 (s, 3H), 3.80-3.62 (m, 5H), 2.46-2.14 (m, 5H), 2.09-1.95 (m, 1H), 1.50 (d, J=7.7 Hz, 9H).

Step c:

To a solution of tert-butyl (2R,4R)-4-(2,3-dichloro-6-methoxyphenyl)-2-(3-methoxy-3-oxopropyl)pyrrolidine-1-carboxylate (0.500 g, 1.16 mmol) in DCM (5 mL) was added TFA (1.50 mL) at room temperature. The reaction was stirred at room temperature for 1 h and then concentrated under reduced pressure. The residue was dissolved in EtOH (15 mL) and TEA (3 mL, 21.6 mmol) was added. The resulting mixture was stirred at 80° C. for 48 h and then concentrated under reduced pressure. The residue was purified by reverse phase chromatography, eluting with 65% ACN in water (plus 0.05% TFA) to afford Intermediate 18 ((6R,7aR)-6-(2,3-dichloro-6-methoxyphenyl)-hexahydropyrrolizin-3-one) as a light yellow solid (0.250 g, 72%): LCMS (ESI) calc'd for C₁₄H₁₅Cl₂NO₂ [M+H]⁺: 300, 302 (3:2), found 300, 302 (3:2); ¹H NMR (400 MHz, CD₃OD) δ 7.41 (d, J=9.0 Hz, 1H), 6.99 (d, J=9.0 Hz, 1H), 4.59-4.47 (m, 1H), 4.21-4.13 (m, 1H), 3.86-3.78 (m, 4H), 3.30-3.22 (m, 1H), 2.86-2.74 (m, 1H), 2.62-2.52 (m, 1H), 2.46-2.35 (m, 1H), 2.24-2.15 (m, 1H), 1.98-1.89 (m, 1H), 1.89-1.79 (m, 1H).

### Example 17. Intermediate 19 ((6R,7aS)-6-(2,3-dichloro-6-methoxyphenyl)-3-oxo-hexahydropyrrolizine-2-carboxylic acid)

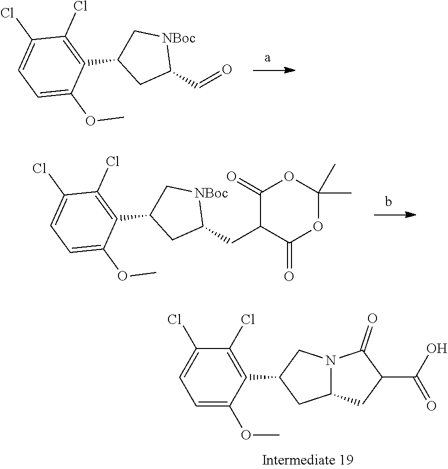

Step a:

To a stirred solution of tert-butyl (2S,4R)-4-(2,3-dichloro-6-methoxyphenyl)-2-formylpyrrolidine-1-carboxylate (Example 7, step c) (1.00 g, 2.67 mmol), 2,2-dimethyl-1,3-dioxane-4,6-dione (Meldrum's acid) (0.380 g, 2.67 mmol) and etidin (0.67 g, 2.67 mmol) in ACN (10 mL) was added L-proline (31.0 mg, 0.27 mmol) at room temperature. The reaction mixture was stirred at room temperature for 4 h under nitrogen atmosphere and then concentrated under reduced pressure. The residue was diluted with MeOH (10 mL) followed by filtration and the filter cake washed with MeOH (2×10 mL). The filtrate was concentrated under reduced pressure. The residue was purified by reverse phase chromatography, eluting with 50% ACN in water (plus 0.05% TFA) to afford tert-butyl (2S,4R)-4-(2,3-dichloro-6-methoxyphenyl)-2-[(2,2-dimethyl-4,6-dioxo-1,3-dioxan-5-yl)methyl]pyrrolidine-1-carboxylate as a light yellow liquid (1.20 g, 89%): LCMS (ESI) calc'd for C₂₃H₂₉Cl₂NO₇ [M+H]⁺: 502, 504 (3:2), found 502, 504 (3:2); ¹H NMR (400 MHz, CDCl₃) δ 7.35 (d, J=8.9 Hz, 1H), 6.79 (d, J=8.9 Hz, 1H), 4.94-4.76 (m, 1H), 4.54-4.44 (m, 1H), 4.08-3.96 (m, 1H), 3.91 (s, 3H), 3.85-3.73 (m, 2H), 2.67-2.55 (m, 1H), 2.55-2.44 (m, 1H), 2.28-2.14 (m, 2H), 1.89 (s, 3H), 1.80 (s, 3H), 1.46 (s, 9H).

Step b:

A solution of tert-butyl (2S,4R)-4-(2,3-dichloro-6-methoxyphenyl)-2-[(2,2-dimethyl-4,6-dioxo-1,3-dioxan-5-yl)methyl]pyrrolidine-1-carboxylate (1.20 g, 2.39 mmol) and TFA (1 mL) in DCM (5 mL) was stirred at room temperature for 1 h and concentrated under reduced pressure. The residue was dissolved with EtOH (3 mL) and basified to pH 8 with TEA (1 mL). The resulting mixture was stirred for 1 h at 80° C. The resulting solution was concentrated under reduced pressure. The residue was purified by reverse phase chromatography, eluting with 35% ACN in water (plus 0.05% TFA) to afford Intermediate 19 ((6R,7aS)-6-(2,3-dichloro-6-methoxyphenyl)-3-oxo-hexahydropyrrolizine-2-carboxylic acid) as a light yellow liquid (0.780 g, 95%): LCMS (ESI) calc'd for C₁₅H₁₅Cl₂NO₄ [M+H]⁺: 344, 346 (3:2), found 344, 346 (3:2); ¹H NMR (300 MHz, CDCl₃) δ 7.34 (d, J=8.8 Hz, 1H), 6.77 (d, J=8.8 Hz, 1H), 4.88-4.62 (m, 1H), 4.58-4.37 (m, 1H), 4.18-4.01 (m, 1H), 4.00-3.63 (m, 5H), 3.41-3.24 (m, 1H), 2.87-2.67 (m, 1H), 2.40-2.09 (m, 2H), 1.99-1.70 (m, 1H).

### Example 18. Intermediate 20 ((6R,7aS)-6-(2,3-dichloro-6-methoxyphenyl)-1-(hydroxymethyl)tetrahydro-1H,3H-pyrrolo[1,2-c]oxazol-3-one)

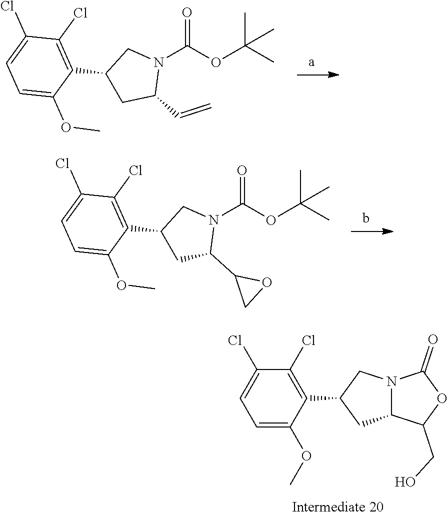

Step a:

To a stirred mixture of tert-butyl (2S,4R)-4-(2,3-dichloro-6-methoxyphenyl)-2-ethenylpyrrolidine-1-carboxylate (Intermediate 16 step a) (3.30 g, 8.86 mmol) in DCM (25 mL) was added m-CPBA (4.59 g, 26.6 mmol) at room temperature. After 2 h the reaction was quenched with saturated aq. Na₂S₂O₃ (50 mL) and extracted with EA (3×30 mL). The combined organic layers were washed with saturated aq. NaHCO₃ (3×30 mL) and brine (2×20 mL) and dried over anhydrous Na₂SO₄. After filtration, the filtrate was concentrated under reduced pressure to afford tert-butyl (2S,4R)-4-(2,3-dichloro-6-methoxyphenyl)-2-(oxiran-2-yl)pyrrolidine-1-carboxylate as a light yellow liquid (3.50 g, crude), which was used directly in the next step without further purification: LCMS (ESI) calc'd C₁₈H₂₃Cl₂NO₄ for [M+Na]⁺: 410, 412 (3:2), found 410, 412 (3:2).

Step b:

A mixture of tert-butyl (2S,4R)-4-(2,3-dichloro-6-methoxyphenyl)-2-(oxiran-2-yl)pyrrolidine-1-carboxylate (3.30 g, 8.50 mmol) and TsOH (0.150 g, 0.850 mmol) in MeOH (25 mL) was stirred at room temperature for 3 h under nitrogen atmosphere. The resulting mixture was concentrated under reduced pressure. The residue was purified by reverse phase chromatography, eluting with 45% ACN in water (plus 0.05% TFA) to afford Intermediate 20 ((6R,7aS)-6-(2,3-dichloro-6-methoxyphenyl)-1-(hydroxymethyl)-tetrahydro-1H-pyrrolo[1,2-c][1,3]oxazol-3-one) as a light yellow solid (1.70 g, 57% over two steps): LCMS (ESI) calc'd for C₁₄H₁₅Cl₂NO₄ [M+H]⁺: 332, 334 (3:2), found 332, 334 (3:2); ¹H NMR (400 MHz, CDCl₃) δ 7.35 (d, J=8.9 Hz, 1H), 6.78 (d, J=8.9, 1H), 4.88-4.48 (m, 1H), 4.42-4.27 (m, 1H), 4.16-3.80 (m, 7H), 3.49-3.36 (m, 1H), 2.29-2.18 (m, 1H), 2.11-1.87 (m, 1H).

Examples 19-108 describe the syntheses of representative compounds of Formula I disclosed herein.

### Example 19. Compound 1 ((8R,9aS)-8-(2,3-dichloro-6-hydroxyphenyl)-2-(2-hydroxyacetyl)-hexahydro-1H-pyrido[1,2-a]pyrazin-4-one)

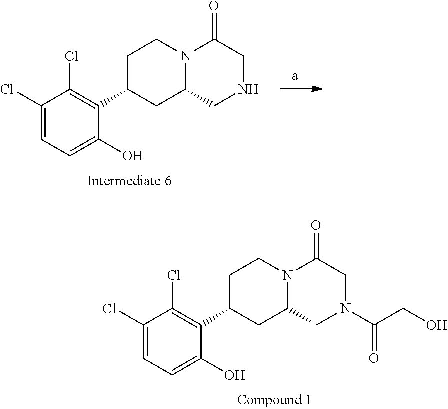

Step a:

To a stirred solution of glycolic acid (9 mg, 0.12 mmol) in DMF (1.00 mL) was added EDCI (32 mg, 0.17 mmol) and HOBT (23 mg, 0.17 mmol) at room temperature. Five mins later, TEA (34 mg, 0.33 mmol) and (8R,9aS)-8-(2,3-dichloro-6-hydroxyphenyl)-octahydropyrido[1,2-a]pyrazin-4-one (Intermediate 6, Example 5) (35 mg, 0.11 mmol) were added. The reaction mixture was stirred at room temperature for 16 h and then concentrated under vacuum. The residue was purified by Prep-HPLC with the following conditions: Column: Xselect CSH OBD, Column 30×150 mm, Sum; Mobile Phase A: Water (0.05% TFA), Mobile Phase B: ACN; Flow rate: 60 mL/min; Gradient: 17% B to 45% B in 7 min; Detector: UV 220 nm; Retention time: 6.97 min. The fractions containing the desired product were combined and concentrated under reduced pressure to afford Compound 1 ((8R,9aR)-8-(2,3-dichloro-6-hydroxyphenyl)-2-(2-hydroxyacetyl)-hexahydro-1H-pyrido[1,2-a]pyrazin-4-one) as an off-white solid (15.6 mg, 38%): LCMS (ESI) calculated for C₁₆H₁₈Cl₂N₂O₄ [M+H]⁺: 373, 375 (3:2), found 373, 375 (3:2). ¹H NMR (400 MHz, Methanol-d4) δ 7.20 (d, J=8.8 Hz, 1H), 6.71 (d, J=8.8 Hz, 1H), 4.77-4.65 (m, 1H), 4.45-3.87 (m, 5H), 3.82-3.42 (m, 3H), 2.82-2.70 (m, 1H), 2.52-2.34 (m, 2H), 1.80-1.58 (m, 2H); (400 MHz, CD₃OD) δ 7.20 (d, J=8.8 Hz, 1H), 6.71 (d, J=8.8 Hz, 1H), 4.74 (d, J=13.3 Hz, 1H), 4.41-3.87 (m, 5H), 3.86-3.39 (m, 3H), 2.76 (td, J=13.2, 3.0 Hz, 1H), 2.54-2.32 (m, 2H), 1.83-1.54 (m, 2H).

### Example 20. Compound 2 ((7R,8aS)-7-(2,3-dichloro-6-hydroxyphenyl)-2-(2-hydroxyethyl)-hexahydropyrrolo[1,2-a]pyrazin-4-one)

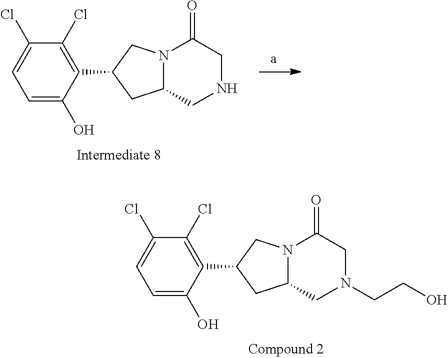 [SMILES: O=C1CNC[C@@H]2C[C@H](c3c(O)ccc(Cl)c3Cl)CN12 ; O=C1CN(CCO)C[C@@H]2C[C@H](c3c(O)ccc(Cl)c3Cl)CN12]

Step a:

To a stirred mixture of (7R,8aS)-7-(2,3-dichloro-6-hydroxyphenyl)-hexahydro-1H-pyrrolo[1,2-a]pyrazin-4-one hydrobromide (Intermediate 8, Example 7) (30 mg, 0.10 mmol) and 2-bromoethanol (50 mg, 0.39 mmol) in ACN (1 mL) was added DIEA (38 mg, 0.30 mmol) dropwise at 0° C. The reaction mixture was stirred for 12 h at 80° C. The reaction was concentrated under reduced pressure. The residue was purified with Prep-IPLC with the following conditions: Column: XBridge Shield RP18 OBD Column 30×150 mm, 5 m; Mobile Phase A: water (plus 0.05% TFA), Mobile Phase B: ACN; Flow rate: 60 mL/min; Gradient: 15% B to 40% B in 7 min; Detector: UV 254/220 nm; Retention time: 6.92 min. The fractions containing the desired product were collected and concentrated under reduced pressure to afford Compound 2 ((7R,8aS)-7-(2,3-dichloro-6-hydroxyphenyl)-2-(2-hydroxyethyl)-hexahydropyrrolo[1,2-a]pyrazin-4-one) as an off-white solid (15 mg, 41%): LCMS (ESI) calculated for C₁₅H₁₈Cl₂N₂O₃ [M+H]⁺: 345, 347 (3:2), found: 345, 347 (3:2); ¹H NMR (400 MHz, CDCl₃) δ 7.18 (d, J=8.8 Hz, 1H), 6.89 (d, J=8.8 Hz, 1H), 4.46-4.29 (m, 1H), 4.24-4.09 (m, 1H), 4.03-3.83 (m, 1H), 3.78-3.62 (m, 3H), 3.39 (t, J=10.6 Hz, 1H), 3.35-3.25 (m, 1H), 3.11 (d, J=16.8 Hz, 1H), 2.85-2.67 (m, 2H), 2.53-2.33 (m, 1H), 2.22 (q, J=11.5 Hz, 1H), 2.16-2.04 (m, 1H).

### Example 21. Compounds 3-11, 14-17, 19-25, 27-29, 31-35, 37-42, 44-45, 47-49, 51, 53-54, 56, and 58-59

The following Compounds were made in analogous fashion to that of Compound 1 (Example 19) or Compound 2 (Example 20), and/or by a method known in the art.

TABLE 1

| Compound | | | |
|---|---|---|---|
| Number | Structure | Chemical Name | MS (M + H)⁺ & ¹H NMR Data |
| 3 | 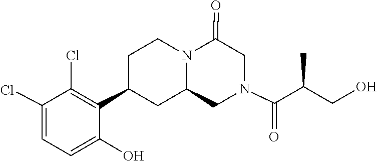 [SMILES: C[C@@H](CO)C(=O)N1CC(=O)N2CC[C@H](c3c(O)ccc(Cl)c3Cl)C[C@@H]2C1] | (8S,9aR)-8-(2,3- dichloro-6-hydroxyphenyl)- 2-((S)-3-hydroxy-2- methylpropanoyl)octahydro-4H-pyrido[1,2-a]pyrazin- 4-one | [M + H]⁺: 401, 403 (3:2); ¹HNMR (400 MHz, CD₃OD) δ 7.20 (d, J = 8.8 Hz, 1H), 6.71 (d, J = 8.8 Hz, 1H), 4.79-4.67 (m, 1H), 4.45-3.89 (m, 3H), 3.84-3.51 (m, 5H), 3.12-3.02 (m, 1H), 2.77 (t, J = 13.1 Hz, 1H), 2.53-2.36 (m, 2H), 1.83-1.57 (m, 2H), 1.11 (d, J = 6.9 Hz, 3H). |
| 4 | 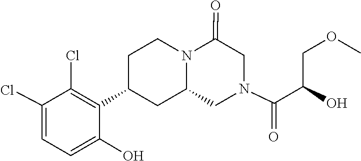 [SMILES: COC[C@@H](O)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2- [(2R)-2-hydroxy-3- methoxypropanoyl]- hexahydro-1H- pyrido[1,2-a]pyrazin-4-one | [M + H]⁺: 417, 419 (3:2); ¹H NMR (400 MHz, CD₃OD) δ 7.20 (d, J = 8.8 Hz, 1H), 6.71 (d, J = 8.7 Hz, 1H), 4.74 (d, J = 13.3 Hz, 1H), 4.67-4.60 (m, 1H), 4.51 (t, J = 18.7 Hz, 1H), 4.38-4.16 (m, 1H), 4.11-3.97 (m, 1H), 3.81-3.50 (m, 5H), 3.36 (s, 3H), 2.75 (t, J = 13.1 Hz, 1H), 2.52-2.32 (m, 2H), 1.81-1.59 (m, 2H). |
| 5 | 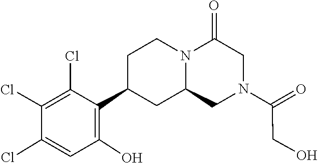 [SMILES: O=C(CO)N1CC(=O)N2CC[C@H](c3c(O)cc(Cl)c(Cl)c3Cl)C[C@@H]2C1] | (8S,9aR)-2-(2- hydroxyacetyl)-8- (2,3,4-trichloro-6- hydroxyphenyl)- hexahydro-1H- pyrido[1,2-a]pyrazin-4-one | [M + H]⁺: 407, 409 (1:1); ¹H NMR (400 MHz, CD₃OD) δ 6.92 (s, 1H), 4.81-4.65 (m, 1H), 4.45-4.22 (m, 3H), 4.19-3.89 (m, 2H), 3.83-3.42 (m, 3H), 2.76 (td, J = 13.0, 2.9 Hz, 1H), 2.54-2.31 (m, 2H), 1.84-1.57 (m, 2H). |
| 6 | 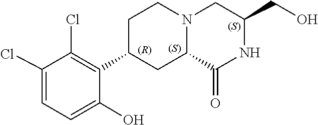 | (3S,8R,9aS)-8-(2,3- dichloro-6- hydroxyphenyl)-3- (hydroxymethyl)- octahydropyrido[1,2- a]pyrazin-1-one | [M + H]⁺: 345, 347 (3:2); ¹H NMR (400 MHz, CD₃OD) δ 7.19 (d, J = 8.8 Hz, 1H), 6.71 (d, J = 8.8 Hz, 1H), 3.79-3.67 (m, 1H), 3.62-3.47 (m, 2H), 3.14-2.99 (m, 2H), 2.74-2.58 (m, 2H), 2.53-2.41 (m, 1H), 2.41-2.29 (m, 2H), 2.19-2.10 (m, 1H), 1.63-1.53 (m, 1H), 1.32 (d, J = 6.6 Hz, 1H). |
| 7 | 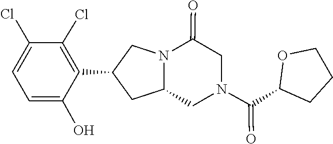 [SMILES: O=C([C@H]1CCCO1)N1CC(=O)N2C[C@@H](c3c(O)ccc(Cl)c3Cl)C[C@H]2C1] | (7R,8aS)-7-(2,3- dichloro-6- hydroxyphenyl)-2- [(2R)-oxolane-2-carbonyl]- hexahydropyrrolo[1,2- a]pyrazin-4-one | [M + H]⁺: 399, 401 (3:2); ¹H NMR (400 MHz, CD₃OD) δ 7.27 (d, J = 8.8 Hz, 1H), 6.77 (d, J = 8.8 Hz, 1H), 4.94-4.59 (m, 2H), 4.59-4.45 (m, 1H), 4.44-4.26 (m, 1H), 4.25-4.13 (m, 1H), 4.06-3.80 (m, 3H), 3.72 (d, J = 18.5 Hz, 1H), 3.59 (t, J = 10.5 Hz, 1H), 3.26-2.68 (m, 1H), 2.44 (q, J = 11.5 Hz, 1H), 2.25-2.14 (m, 3H), 2.01-1.93 |
| | | | (m, 2H). |
| 8 | 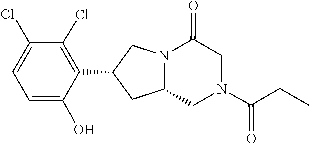 [SMILES: CCC(=O)N1CC(=O)N2C[C@@H](c3c(O)ccc(Cl)c3Cl)C[C@H]2C1] | (7R,8aS)-7-(2,3-dichloro-6- hydroxyphenyl)- 2-pro-pionylhexahydropyrrolo [1,2-a]pyrazin-4(1H)-one | [M + H]⁺: 357, 359 (3:2); ¹H NMR (400 MHz, CD₃OD) δ 7.27 (d, J = 8.8 Hz, 1H), 6.77 (d, J = 9.0 Hz, 1H), 4.64 (d, J = 18.5 Hz, 1H), 4.44-4.26 (m, 2H), 4.24-4.14 (m, 1H), 4.09-3.67 (m, 2H), 3.59 (t, J = 10.5 Hz, 1H), 3.23-2.68 (m, 1H), 2.58-2.36(m, 3H), 2.26-2.14 (m, 1H), 1.22-1.07 (m, 3H). |
| 9 | 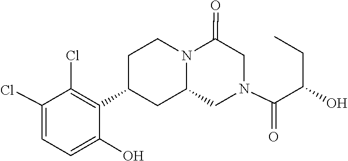 [SMILES: CC[C@H](O)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2- [(2S)-2- hydroxybutanoyl]- hexahydro-1H- pyrido[1,2-a]pyrazin- 4-one | [M + H]⁺: 401, 403 (3:2); ¹H NMR (400 MHz, CD₃OD) δ 7.20 (d, J = 8.7 Hz, 1H), 6.71 (d, J = 8.8 Hz, 1H), 4.78-4.66 (m, 1H), 4.45-3.89 (m, 4H), 3.87-3.53 (m, 3H), 2.83-2.70 (m, 1H), 2.54-2.34 (m, 2H), 1.86- 1.55 (m, 4H), 1.10-0.92 (m, 3H). |
| 10 | 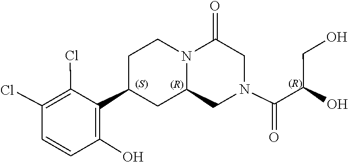 | (8S,9aR)-rel-8- (2,3-dichloro-6-hydroxyphenyl)- 2-[(2R)-2,3-dihydroxypropanoyl]-hexahydro-1H- pyrido[1,2-a]pyrazin- 4-one | [M + H]⁺_{:} 403, 405 (3:2); ¹H NMR (400 MHz, CD₃OD) δ 7.20 (d, J = 8.8 Hz, 1H), 6.71 (d, J = 8.8 Hz, 1H), 4.80-4.66 (m, 1H), 4.62-3.91 (m, 4H), 3.83-3.46 (m, 5H), 2.82-2.68 (m, 1H), 2.57-2.33 (m, 2H), 1.91- 1.58 (m, 2H). |
| 11 | 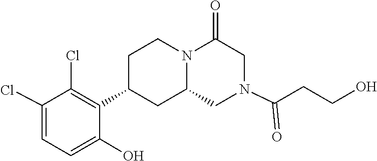 [SMILES: O=C(CCO)N1CC(=O)N2CC[C@@H](c3c(O)ccc(Cl)c3Cl)C[C@H]2C1] | (8R,9aS)-8-(2,3-dichloro-6- hydroxyphenyl)-2-(3- hydroxypropanoyl)octahydro- 4H-pyrido[1,2-a]pyrazin- 4-one | [M + H]⁺: 387, 389 (3:2); ¹H NMR (400 MHz, CD₃OD) δ 7.20 (dd, J = 8.8, 2.3 Hz, 1H), 6.71 (d, J = 8.8 Hz, 1H), 4.78-4.69 (m, 1H), 4.45-4.00 (m, 3H), 3.85 (dd, J = 7.1, 5.4 Hz, 2H), 3.82-3.52 (m, 3H), 2.86-2.56 (m, 3H), 2.50-2.36 (m, 2H), 1.78- 1.62 (m, 2H). |
| 14 | 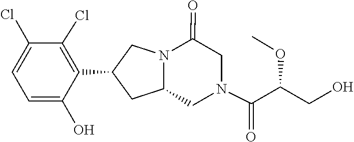 [SMILES: CO[C@H](CO)C(=O)N1CC(=O)N2C[C@@H](c3c(O)ccc(Cl)c3Cl)C[C@H]2C1] | (7R,8aS)-7-(2,3- dichloro-6- hydroxyphenyl)-2- [(2R)-3-hydroxy-2- methoxypropanoyl]- hexahydropyrrolo[1,2- a]pyrazin-4-one | [M + H]⁺: 403, 405 (3:2); ¹H NMR (400 MHz, CD₃OD) δ 7.27 (d, J = 8.8 Hz, 1H), 6.77 (d, J = 8.8 Hz, 1H), 4.78-4.69 (m, 2H), 4.48-4.24 (m, 2H), 4.19 (t, J = 10.4 Hz, 1H), 4.14- 3.81 (m, 1H), 3.81-3.47 (m, 4H), 3.40 (d, J = 8.1 Hz, 3H), 3.25-2.74 (m, 1H), 2.44 (q, J = 11.4 Hz, 1H), 2.24-2.17 (m, 1H). |
| 15 | 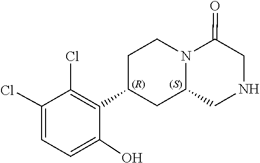 | (8R,9aS)-8-(2,3-dichloro-6- hydroxyphenyl- octahydropyrido[1,2- a]pyrazin-4-one | [M + H]⁺: 315, 317 (3:2); ¹H NMR (400 MHz, CD₃OD) δ 7.20 (d, J = 8.8 Hz, 1H), 6.71 (d, J = 8.8 Hz, 1H), 4.82-4.71 (m, 1H), 3.76-3.57 (m, 1H), 3.58-3.49 (m, 1H), 3.43 (s, 2H), 3.23 (dd,J = 13.4, 5.1Hz, 1H), 2.81- 2.62 (m, 2H), 2.54-2.30 (m, 2H), 1.70-1.49 (m, 2H). |
| 16 | 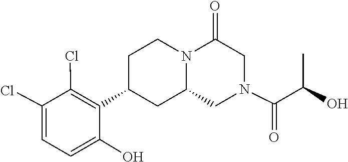 [SMILES: C[C@@H](O)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2-[(2R)-2- hydroxypropanoyl]- hexahydro-1H- pyrido[1,2-a]pyrazin-4-one | [M + H]⁺: 387, 389 (3:2); ¹H NMR (300 MHz, CD₃OD) δ 7.20 (d, J = 8.8 Hz, 1H), 6.71 (d, J = 8.8 Hz, 1H), 4.79-4.67 (m, 1H), 4.65-3.91 (m, 4H), 3.82-3.54 (m, 3H), 2.76 (t, J = 13.2 Hz, 1H), 2.52-2.35 (m, 2H), 1.81-1.55 (m, 2H), 1.35 (d, J = 6.4 Hz, 3H). |
| 17 | 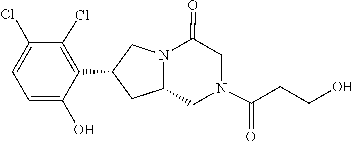 [SMILES: O=C(CCO)N1CC(=O)N2C[C@@H](c3c(O)ccc(Cl)c3Cl)C[C@H]2C1] | (7R,8aS)-7-(2,3-dichloro-6- hydroxyphenyl)-2-(3- hydroxypropanoyl)hexa- hydropyrrolo[1,2- a]pyrazin-4(1H)-one | [M + H]⁺: 373, 375 (3:2); ¹H NMR (400 MHz, CD₃OD) δ 7.27 (d, J = 8.8 Hz, 1H), 6.98-6.65 (m, 1H), 4.77-4.58 (m, 1H), 4.58-4.25 (m, 2H), 4.25-4.13 (m, 1H), 4.13-3.66 (m, 4H), 3.59 (t, J = 10.5 Hz, 1H), 3.25-3.12 (m, 1H), 2.85-2.54 (m, 2H), 2.45 (q, J = 11.4 Hz, 1H), 2.24- 2.18 (m, 1H). |
| 19 | 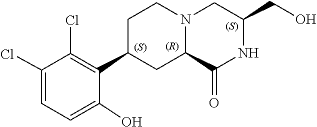 | (3S,8S,9aR)-8-(2,3- dichloro-6- hydroxyphenyl)-3- (hydroxymethyl)- octahydropyrido[1,2- a]pyrazin-1-one | [M + H]⁺: 345, 347 (3:2); ¹H NMR (400 MHz, CD₃OD) δ 7.18 (d, J = 8.8 Hz, 1H), 6.71 (d, J = 8.8 Hz, 1H), 3.79-3.65 (m, 2H), 3.55-3.41 (m, 1H), 3.41-3.35 (m, 1H), 3.11-3.00 (m, 2H), 2.71-2.56 (m, 3H), 2.47- 2.25 (m, 2H), 2.13-2.05 (m, 1H), 1.58-1.50 (m, 1H). |
| 20 | 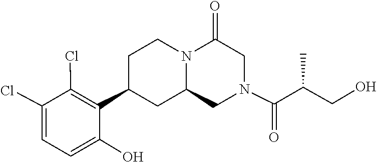 [SMILES: C[C@H](CO)C(=O)N1CC(=O)N2CC[C@H](c3c(O)ccc(Cl)c3Cl)C[C@@H]2C1] | (8S,9aR)-8-(2,3- dichloro-6- hydroxyphenyl)-2- ((R)-3-hydroxy-2- methylpropanoyl)octahydro- 4H-pyrido[1,2-a]pyrazin- 4-one | [M + H]⁺: 401, 403 (3:2). ¹H NMR (400 MHz, CD₃OD) δ 7.21 (d, J = 8.8 Hz, 1H), 6.71 (d, J = 8.7 Hz, 1H), 4.73 (d, J = 13.1 Hz, 1H), 4.46-3.99 (m, 3H), 3.84-3.51 (m, 5H), 3.19- 2.99 (m, 1H), 2.76 (t, J = 13.1 Hz, 1H), 2.53-2.34 (m, 2H), 1.81-1.58 (m, 2H), 1.12-1.03 (m, 3H). |
| 21 | 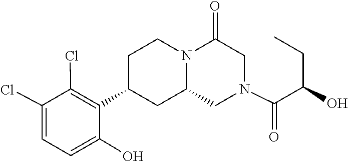 [SMILES: CC[C@@H](O)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2-[(2R)-2- hydroxybutanoyl]- hexahydro-1H- pyrido[1,2-a]pyrazin-4-one | [M + H]⁺: 401, 403 (3:2); ¹H NMR (400 MHz, CD₃OD) δ 7.20 (d, J = 9.0 Hz, 1H), 6.71 (d, J = 8.8 Hz, 1H), 4.79-4.67 (m, 1H), 4.55-3.91 (m, 4H), 3.84-3.53 (m, 3H), 2.83-2.68 (m, 1H), 2.55-2.34 (m, 2H), 1.85- 1.54 (m, 4H), 1.09-0.89 (m, 3H). |
| 22 | 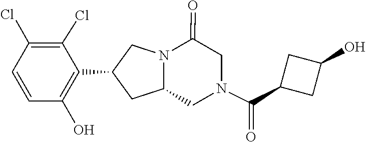 [SMILES: O=C1CN(C(=O)[C@H]2C[C@@H](O)C2)C[C@@H]2C[C@H](c3c(O)ccc(Cl)c3Cl)CN12] | (7R,8aS)-7-(2,3-dichloro-6- hydroxyphenyl)-2-(3- hydroxycyclobutane-1- carbonyl)hexahydropyrrolo [1,2-a]pyrazin- 4(1H)-one isomer 2 | [M + H]⁺: 399, 401 (3:2); ¹H NMR (400 MHz, CD₃OD) δ 7.27 (d, J = 8.8 Hz, 1H), 6.77 (d, J = 8.8 Hz, 1H), 4.90-4.59 (m, 1H), 4.45-4.24 (m, 2H), 4.24-4.09 (m, 2H), 4.11-3.68 (m, 2H), 3.58 (t, J = 10.5 Hz, 1H), 3.29-3.11 (m, 1H), 3.01-2.84 (m, 1H), 2.84-2.72 (m, 1H), 2.65-2.36 (m, 2H), 2.29-2.06 (m, 3H). |
| 23 | 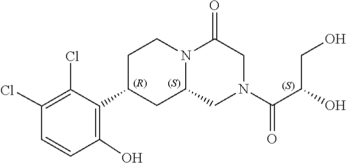 | (8R,9aS)-8-(2,3-dichloro-6- hydroxyphenyl)-2-[(2S)-2,3- dihydroxypropanoyl]-hexahydro-1H-pyrido[1,2-a]pyrazin-4-one | [M + H]⁺: 403, 405 (3:2); ¹H NMR (400 MHz, CD₃OD) δ 7.20 (d, J = 8.8 Hz, 1H), 6.71 (d, J = 8.8 Hz, 1H), 4.77-4.64 (m, 1H), 4.61-4.39 (m, 2H), 4.37-4.04 (m, 2H), 3.84-3.52 (m, 5H), 2.83-2.67 (m, 1H), 2.58- 2.31 (m, 2H), 1.82-1.56 (m, 2H). |
| 24 | 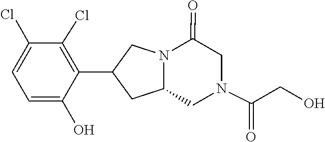 [SMILES: O=C(CO)N1CC(=O)N2CC(c3c(O)ccc(Cl)c3Cl)C[C@H]2C1] | (8aS)-7-(2,3-dichloro-6- hydroxyphenyl)-2-(2- hydroxyacetyl)- hexahydropyrrolo[1,2- a]pyrazin-4-one | [M + H]⁺: 359, 361 (3:2); ¹H NMR (400 MHz, CD₃OD) δ 7.27 (d, J = 8.8 Hz, 1H), 6.77 (d, J = 8.8 Hz, 1H), 4.68-4.59 (m, 1H), 4.39-4.14 (m, 5H), 4.05-3.73 (m, 2H), 3.60 (t, J = 10.5 Hz, 1H), 3.19-2.75 (m, 1H), 2.50-2.37 (m, 1H), 2.26-2.12 (m, 1H). |
| 25 | 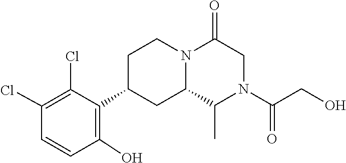 | (8R,9aS)-8-(2,3-dichloro-6- hydroxypheny1)-2-(2- hydroxyacetyl)-1- methyl-hexahydro- 1H-pyrido[1,2- a]pyrazin-4-one | [M + H]⁺: 387, 389 (3:2); ¹H NMR (400 MHz, CD₃OD) δ 7.21 (d, J = 8.7 Hz, 1H), 6.73 (d, J = 8.8 Hz, 1H), 4.82-4.56 (m, 2H), 4.46-3.53 (m, 6H), 2.90-2.77 (m, 1H), 2.55-2.38 (m, 2H), 1.73-1.65 (m, 2H), 1.40- 1.16 (m, 3H). |
| 27 | 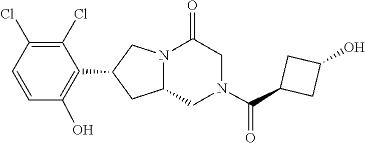 [SMILES: O=C1CN(C(=O)[C@H]2C[C@H](O)C2)C[C@@H]2C[C@H](c3c(O)ccc(Cl)c3Cl)CN12] | (7R,8aS)-7-(2,3-dichloro-6- hydroxyphenyl)-2-(3- hydroxycyclobutane-1- carbonyl)hexa- hydropyrrolo[1,2-a]pyrazin- 4(1H)-one isomer 1 | [M + H]⁺: 399, 401 (3:2); ¹H NMR (400 MHz, CD₃OD) δ 7.27 (d, J = 8.8 Hz, 1H), 6.77 (d, J = 8.8 Hz, 1H), 4.87-4.60 (m, 1H), 4.41-4.26 (m, 2H), 4.26-4.11 (m, 2H), 4.05-3.68 (m, 2H), 3.58 (t, J = 10.5 Hz, 1H), 3.44-3.32 (m, 1H), 3.19-2.70 (m, 1H), 2.63-2.40 (m, 3H), 2.34-2.15 (m, 3H). |
| 28 | 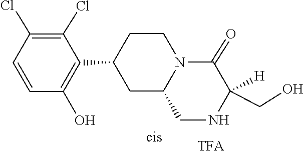 [SMILES: O=C1[C@@H](CO)NC[C@@H]2C[C@H](c3c(O)ccc(Cl)c3Cl)CCN12] | (8R,9aS)-rel-8-(2,3- dichloro-6- hydroxyphenyl)-3-((3R)- hydroxymethyl- octahydropyrido[1,2- a]pyrazin-4-one trifluoroacetic acid | [M + H]⁺: 345, 347 (3:2); ¹H NMR (400 MHz, CD₃OD) δ 7.23 (dd, J = 8.8, 1.5 Hz, 1H), 6.73 (d, J = 8.8 Hz, 1H), 4.83-4.73 (m, 1H), 4.26-3.95 (m, 3H), 3.91-3.45 (m, 4H), 2.89- 2.72 (m, 1H), 2.67-2.35 (m, 2H), 1.88-1.65 (m, 2H); ¹⁹F NMR (376 MHz, CD₃OD) δ-76.99. |
| 29 | 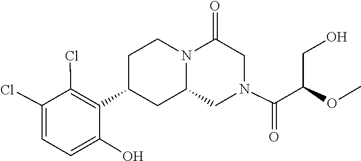 [SMILES: CO[C@H](CO)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2- [(2R)-3-hydroxy-2- methoxypropanoyl]- hexahydro-1H-pyrido[1,2-a]pyrazin-4-one | [M + H]⁺: 417, 419 (3:2); ¹H NMR (400 MHz, CD₃OD) δ 7.20 (dd, J = 8.7, 2.9 Hz, 1H), 6.78-6.66 (m, 1H), 4.73 (t, J = 13.6 Hz, 1H), 4.58-4.47 (m, 1H), 4.45-4.31 (m, 1H), 4.31- 4.23 (m, 1H), 4.08-3.87 (m, 1H), 3.87-3.57 (m, 5H), 3.39 (s, 3H), 2.77 (td, J = 13.1,3.0 Hz, 1H), 2.54-2.35 (m, 2H), 1.81-1.56 (m, 2H). |
| 31 | 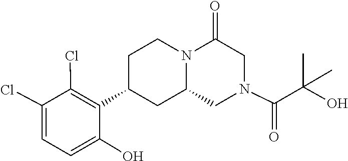 [SMILES: CC(C)(O)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2-(2- hydroxy-2- methylpropanoyl)octahydro- 4H-pyrido[1,2-a]pyrazin- 4-one | [M + H]⁺: 401, 403 (3:2); ¹H NMR (400 MHz, CD₃OD) δ 7.20 (d, J = 8.8 Hz, 1H), 6.71 (d, J = 8.8 Hz, 1H), 4.79-4.52 (m, 3H), 4.20-3.70 (m, 3H), 3.70-3.56 (m, 1H), 2.75 (td, J = 13.1, 3.0 Hz, 1H), 2.53-2.33 (m, 2H), 1.71-1.61 (m, 2H), 1.45 (d, J =7.8 Hz, 6H). |
| 32 | 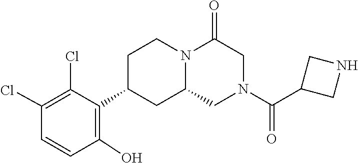 [SMILES: O=C(C1CNC1)N1CC(=O)N2CC[C@@H](c3c(O)ccc(Cl)c3Cl)C[C@H]2C1] | (8S,9aR)-rel-2- (azetidine-3- carbonyl)-8-(2,3-dichloro-6- hydroxyphenyl)- hexahydro-1H- pyrido[1,2-a]pyrazin-4-one | [M + H]⁺: 398, 400 (3:2); ¹H NMR (400 MHz, CD₃OD) δ 7.21 (d, J = 8.8 Hz, 1H), 6.71 (d, J = 8.8 Hz, 1H), 4.71 (t, J = 13.6 Hz, 1H), 4.36-4.16 (m, 1H), 4.04 (s, 1H), 4.02-3.69 (m, 7H), 3.68-3.55 (m, 1H), 3.49-3.37 (m, 1H), 2.81-2.69 (m, 1H), 2.51- 2.33 (m, 2H), 1.78-1.56 (m, 2H). |
| 33 | 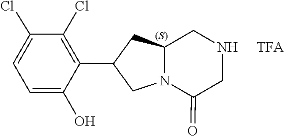 | (8aS)-7-(2,3-dichloro-6- hydroxyphenyl)- hexahydro-1H- pyrrolo[1,2-a]pyrazin-4-one trifluoroacetic acid | [M + H]⁺: 301, 303 (3:2); ¹H NMR (400 MHz, CD₃OD) δ 7.28 (d, J = 8.8 Hz, 1H), 6.78 (d, J = 8.8 Hz, 1H), 4.42-4.28 (m, 1H), 4.22 (dd, J = 11.5, 9.2 Hz, 1H), 4.11-3.98 (m, 1H), 3.88- 3.76 (m, 3H), 3.64 (t, J = 10.6 Hz, 1H), 3.14-3.05 (m, 1H), 2.47 (q, J = 11.4 Hz, 1H), 2.34-2.18 (m, 1H); |
| 34 | 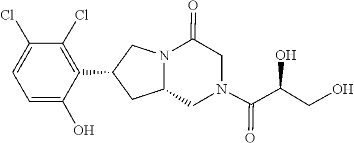 [SMILES: O=C([C@@H](O)CO)N1CC(=O)N2C[C@@H](c3c(O)ccc(Cl)c3Cl)C[C@H]2C1] | (7R,8aS)-7-(2,3-dichloro-6- hydroxyphenyl)-2-[(2S)-2,3- dihydroxypropanoyl]- hexahydropyrrolo [1,2-a]pyrazin-4-one | [M + H]+: 389, 391 (3:2); ¹H NMR (400 MHz, CD₃OD) δ 7.27 (d, J = 8.8 Hz, 1H), 6.77 (d, J = 8.8 Hz, 1H), 4.84-4.44 (m, 3H), 4.44-3.95 (m, 3H), 3.95-3.67 (m, 3H), 3.59 (t, J = 10.6 Hz, 1H), 3.27-2.72 (m, 1H), 2.44 (q, J = 11.4 Hz, 1H), 2.23-2.16 (m, 1H). |
| 35 | 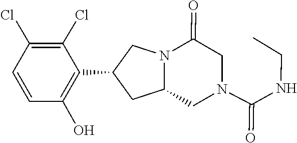 [SMILES: CCNC(=O)N1CC(=O)N2C[C@@H](c3c(O)ccc(Cl)c3Cl)C[C@H]2C1] | (7R,8aS)-7-(2,3-dichloro-6- hydroxyphenyl)-N- ethyl-4-oxo- hexahydropyrrolo[1,2 -a]pyrazine-2- carboxamide | [M + H]⁺: 372, 374 (3:2); ¹H NMR (400 MHz, CD₃OD) δ 7.26 (d, J = 8.8 Hz, 1H), 6.77 (d, J = 8.8 Hz, 1H), 4.45 (dd, J = 13.1, 3.7 Hz, 1H),4.40- 4.26 (m, 2H), 4.17 (dd,J = 11.4, 9.4 Hz, 1H), 3.95-3.84 (m, 1H), 3.79 (d, J = 17.6 Hz, 1H), 3.63-3.54 (m, 1H), 3.23 (q, J = 12 Hz, 2H), 2.84 (dd, J = 13.2, 10.4 Hz, 1H), 2.42 (q, J = 11.5 |
| | | | Hz, 1H), 2.23-2.12 (m, 1H), |
| | | | 1.15 (t, J = 12 Hz, 3H). |
| 37 | 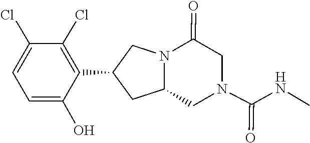 [SMILES: CNC(=O)N1CC(=O)N2C[C@@H](c3c(O)ccc(Cl)c3Cl)C[C@H]2C1] | (7R,8aS)-7-(2,3-dichloro-6- hydroxyphenyl)-N- methyl-4-oxo- hexahydropyrrolo[1,2- a]pyrazine-2- carboxamide | [M + H]⁺: 358, 360 (3:2); ¹H NMR (400 MHz, CD₃OD) δ 7.26 (d, J = 8.8 Hz, 1H), 6.76 (d, J = 8.8 Hz, 1H), 4.43 (dd, J = 13.0, 3.6 Hz, 1H), 4.39- 4.24 (m, 2H), 4.22-4.12 (m, 1H), 3.94-3.83 (m, 1H), 3.79 (d, J = 17.4 Hz, 1H), 3.63-3.54 (m, 1H), 2.84 (dd, J = 13.1, 10.4 Hz, 1H), 2.77 (s, 3H), 2.42 (q, J = 11.4 Hz, 1H), 2.22-2.12 |
| | | | (m, 1H). |
| 38 | 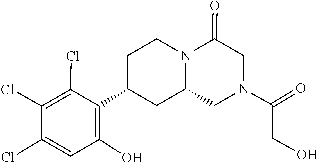 [SMILES: O=C(CO)N1CC(=O)N2CC[C@@H](c3c(O)cc(Cl)c(Cl)c3Cl)C[C@H]2C1] | (8R,9aS)-2-(2- hydroxyacetyl)-8- (2,3,4-trichloro-6- hydroxyphenyl)octahydro- 4H-pyrido[1,2-a]pyrazin-4- one | [M + H]⁺: 407, 409 (1:1); ¹H NMR (400 MHz, CD₃OD) δ 6.92 (s, 1H), 4.76-4.72 (m, 1H), 4.45-4.22 (m, 3H), 4.20-3.87 (m, 2H), 3.87-3.44 (m, 3H), 2.76 (td, J = 13.1, 2.9 Hz, 1H), 2.53-2.30 (m, 2H), 1.76-1.62 (m, 2H). |
| 39 | 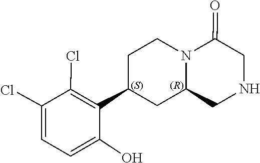 | (8S,9aR)-8-(2,3-dichloro-6- hydroxyphenyl- octahydropyrido[1,2- a]pyrazin-4-one | [M + H]⁺: 315, 317 (3:2); ¹H NMR (400 MHz, CD₃OD) δ 7.20 (d, J = 8.7 Hz, 1H), 6.71 (d, J = 8.8 Hz, 1H), 4.82-4.73 (m, 1H), 3.76-3.60 (m, 1H), 3.59-3.48 (m, 1H), 3.43 (s, 2H), 3.24 (dd, J = 13.4, 5.1 Hz, 1H), 2.82- 2.63 (m, 2H), 2.57-2.34 (m, 2H), 1.69-1.59 (m, 2H). |
| 40 | 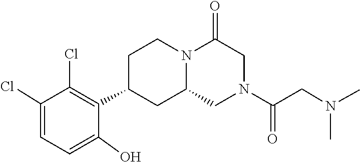 [SMILES: CN(C)CC(=O)N1CC(=O)N2CC[C@@H](c3c(O)ccc(Cl)c3Cl)C[C@H]2C1] | (8R,9aS)-8-(2,3-dichloro-6- hydroxyphenyl)-2-[2- (dimethylamino)acetyl]- hexahydro-1H-pyrido[1,2-a]pyrazin-4-one | [M + H]⁺: 400, 402 (3:2); ¹H NMR (400 MHz, CD₃OD) δ 7.20 (dd, J = 8.8, 2.9 Hz, 1H), 6.71 (d, J = 8.8 Hz, 1H), 4.73 (d, J = 13.1 Hz, 1H), 4.57- 4.14 (m, 2H), 4.09-3.87 (m, 1H), 3.84-3.56 (m, 3H), 3.31-3.10 (m, 2H), 2.77 (td, J = 13.2, 3.0 Hz, 1H), 2.54-2.37 (m, 2H), 2.31 (d, J = 8.3 Hz, 6H), 1.77-1.61 (m, 2H). |
| 41 | 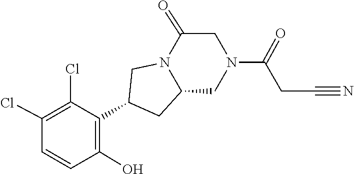 [SMILES: N#CCC(=O)N1CC(=O)N2C[C@@H](c3c(O)ccc(Cl)c3Cl)C[C@H]2C1] | 3-[(7R,8aS)-7-(2,3- dichloro-6- hydroxypheny1)-4-oxo- hexahydropyrrolo[1,2- a]pyrazin-2-yl]-3- oxopropanenitrile | [M + H]⁺ 368, 370 (3:2); ¹H NMR (400 MHz, DMSO) δ 10.40 (s, 1H), 7.35 (d, J = 8.8 Hz, 1H), 6.83 (d, J = 8.8 Hz, 1H), 4.73-4.27 (m, 1H), 4.23- 3.85 (m, 6H), 3.79-3.60 (m, 1H), 3.53-3.40 (m, 1H), 3.14-2.60 (m, 1H), 2.28-2.02 (m, 2H). |
| 42 | 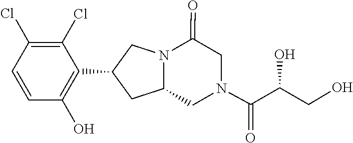 [SMILES: O=C([C@H](O)CO)N1CC(=O)N2C[C@@H](c3c(O)ccc(Cl)c3Cl)C[C@H]2C1] | (7R,8aS)-7-(2,3-dichloro-6-hydroxyphenyl)-2-((R)-2,3- dihydroxypropanoyl) hexahydropyrrolo[1,2- a]pyrazin-4(1H)-one | [M + H]⁺: 389, 391 (3:2); ¹H NMR (400 MHz, CD₃OD) δ 7.27 (d, J = 8.8 Hz, 1H), 6.77 (d, J = 8.8 Hz, 1H), 4.77-4.60 (m, 2H), 4.59-4.48 (m, 1H), 4.43-4.24 (m, 1H), 4.19 (t, J = 10.4 Hz, 1H), 4.15-3.85 (m, 1H), 3.85-3.65 (m, 3H), 3.59 (t, J = 10.7 Hz, 1H), 3.22-2.75 (m, 1H), 2.44 (q, J = 11.4 Hz, 1H), 2.25-2.15 (m, 1H) |
| 44 | 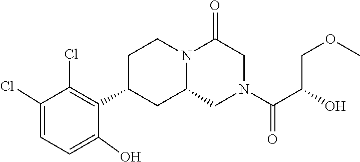 [SMILES: COC[C@H](O)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2- [(2S)-2-hydroxy-3- methoxypropanoyl]- hexahydro-1H- pyrido[1,2-a]pyrazin-4-one | [M + H]⁺: 417, 419 (3:2); ¹H NMR (400 MHz, CD₃OD) δ 7.20 (d, J = 8.8 Hz, 1H), 6.71 (d, J = 8.8 Hz, 1H), 4.73 (d, J = 13.3 Hz, 1H), 4.65 (t, J = 5.8 Hz, 1H), 4.46-4.33 (m, 1H), 4.17 (dd, J = 39.8, 16.0 Hz, 1H), 3.99- 3.51 (m, 6H), 3.43-3.35 (m, 3H), 2.76 (td, J = 13.1, 3.0 Hz, 1H), 2.52- 2.34 (m, 2H), 1.79-1.58 (m, 2H). |
| 45 | 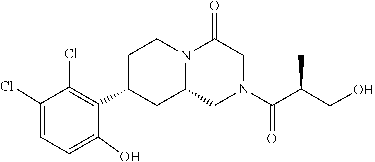 [SMILES: C[C@@H](CO)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2- ((S)-3-hydroxy-2- methylpropanoyl)octahydro- 4H-pyrido[1,2-a]pyrazin- 4-one | [M + H]⁺ 401, 403 (3:2); ¹H NMR (400 MHz, CD₃OD) δ 7.21 (d, J = 8.9 Hz, 1H), 6.71 (d, J = 8.8 Hz, 1H), 4.78-4.67 (m, 1H), 4.47-4.26 (m, 1H), 4.26-3.98 (m, 2H), 3.84-3.52 (m, 5H), 3.21-2.94 (m, 1H), 2.76 (t, J = 13.0 Hz, 1H), 2.54-2.32 (m, 2H), 1.80-1.56 (m, 2H), 1.13-0.97 (m, 3H). |
| 47 | 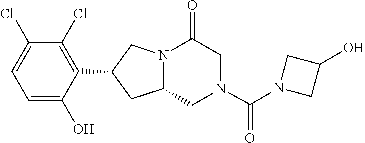 [SMILES: O=C(N1CC(O)C1)N1CC(=O)N2C[C@@H](c3c(O)ccc(Cl)c3Cl)C[C@H]2C1] | (7R,8aS)-7-(2,3- dichloro-6- hydroxypheny1)-2-(3- hydroxyazetidine-1- carbonyl)- hexahydropyrrolo[1,2- a]pyrazin-4-one | [M + H]⁺: 400, 402 (3:2); ¹H NMR (400 MHz, CD₃OD) δ 7.26 (d, J = 8.8 Hz, 1H), 6.76 (d, J = 8.8 Hz, 1H), 4.60-4.49 (m, 1H), 4.38-4.12 (m, 6H), 3.95-3.79 (m, 4H), 3.62-3.52 (m, 1H), 2.90 (dd, J = 13.2, 10.4 Hz, 1H), 2.40 (q, J = 11.5 Hz, 1H), 2.23- 2.12 (m, 1H). |
| 48 | 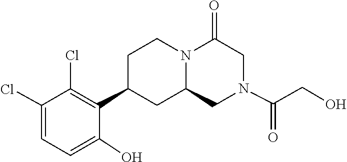 [SMILES: O=C(CO)N1CC(=O)N2CC[C@H](c3c(O)ccc(Cl)c3Cl)C[C@@H]2C1] | (8S,9aR)-8-(2,3- dichloro-6- hydroxyphenyl)-2-(2- hydroxyacetyl)- hexahydro-1H- pyrido[1,2-a]pyrazin- 4-one | [M + H]⁺: 373, 375 (3:2); ¹H NMR (400 MHz, CD₃OD) δ 7.20 (d, J = 8.7 Hz, 1H),6.71 (d, J = 8.8Hz, 1H), 4.74 (d, J = 13.2 Hz, 1H), 4.44-3.87 (m, 5H), 3.82-3.41 (m, 3H), 2.76 (td, J = 13.2, 3.0 Hz, 1H), 2.53-2.34 (m, 2H), 1.78-1.59 (m, 2H). |
| 49 | 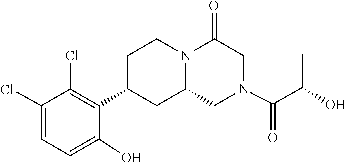 [SMILES: C[C@H](O)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2-[(2S)-2- hydroxypropanoyl]- hexahydro-1H- pyrido[1,2-a]pyrazin- 4-one | [M + H]⁺: 387, 389 (3:2); ¹H NMR (400 MHz, CD₃OD) δ 7.20 (d, J = 8.8 Hz, 1H), 6.71 (d, J = 8.8 Hz, 1H), 4.79-4.67 (m, 1H), 4.60 (q, J = 6.6 Hz, 1H), 4.40-3.91 (m, 3H), 3.83- 3.54 (m, 3H), 2.77 (t, J = 12.8 Hz, 1H), 2.56-2.35 (m, 2H), 1.81-1.55 (m, 2H), 1.36 (d, J = 6.6 Hz, 3H). |
| 51 | 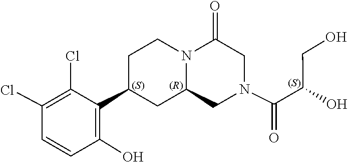 | (8S,9aR)-8-(2,3-dichloro-6- hydroxyphenyl)-2-[(2S)-2,3- dihydroxypropanoyl]- hexahydro-1H- pyrido[1,2-a]pyrazin- 4-one | [M + H]⁺: 403, 405 (3:2); ¹H NMR (400 MHz, CD₃OD) δ 7.20 (d, J = 8.8 Hz, 1H), 6.71 (d, J = 8.7 Hz, 1H), 4.78-4.68 (m, 1H), 4.59-4.38 (m, 3H), 4.31-3.95 (m, 1H), 3.82-3.55 (m, 5H), 2.83-2.69 (m, 1H), 2.56- 2.30 (m, 2H), 1.81-1.58 (m, 2H). |
| 53 | 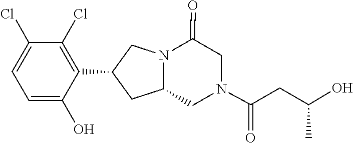 [SMILES: C[C@@H](O)CC(=O)N1CC(=O)N2C[C@@H](c3c(O)ccc(Cl)c3Cl)C[C@H]2C1] | (7R,8aS)-7-(2,3-dichloro-6- hydroxyphenyl)-2-[(3R)-3- hydroxybutanoyl]- hexahydropyrrolo[1,2- a]pyrazin-4-one | [M + H]⁺: 387, 389 (3:2); ¹H NMR (400 MHz, CD₃OD) δ 7.27 (d, J = 8.8, 1H), 6.77 (d, J = 8.6 Hz, 1H), 4.66 (d, J = 18.7 Hz, 1H), 4.58-4.41 (m, 1H), 4.38-4.28 (m, 1H), 4.27-4.13 (m, 2H), 4.08-3.71 (m, 2H), 3.64-3.54 (m, 1H), 3.24-3.12 (m, 1H), 2.78-2.52 (m, 2H), 2.50-2.37 (m, 1H), 2.26-2.14 (m, 1H), 1.27 (d, J = 6.2 Hz, 3H). |
| 54 | 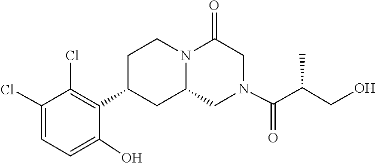 [SMILES: C[C@H](CO)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2- ((R)-3-hydroxy-2- methylpropanoyl)octahydro- 4H-pyrido[1,2-a]pyrazin- 4-one | [M + H]⁺: 401, 403 (3:2); ¹H NMR (400 MHz, CD₃OD) δ 7.20 (d, J = 8.7 Hz, 1H), 6.71 (d, J = 8.7 Hz, 1H), 4.78-4.65 (m, 1H), 4.46-3.89 (m, 3H), 3.85-3.51 (m, 5H), 3.13-3.02 (m, 1H), 2.77 (t, J = 13.2 Hz, 1H), 2.53-2.33 (m, 2H), 1.85-1.56 (m, 2H), 1.11 (d, J = 6.8 Hz, 3H). |
| 56 | 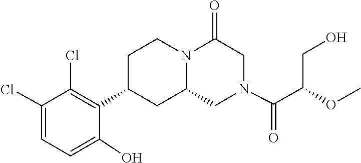 [SMILES: CO[C@@H](CO)C(=O)N1CC(=O)N2CC[C@@H](c3c(O)ccc(Cl)c3Cl)C[C@H]2C1] | (8R,9aS)-8-(2,3-dichloro-6- hydroxyphenyl)-2- [(2S)-3-hydroxy-2- methoxypropanoyl]- hexahydro-1H- pyrido[1,2-a]pyrazin-4-one | [M + H]⁺: 417, 419 (3:2); ¹H NMR (400 MHz, CD₃OD) δ 7.20 (d, J = 8.8 Hz, 1H), 6.70 (d, J = 8.8 Hz, 1H), 4.79-4.68 (m, 1H), 4.67-4.37 (m, 1H), 4.37-4.25 (m, 1H), 4.23-4.12 (m, 1H), 4.00-3.57 (m, 6H), 3.36 (d, J = 31.7 Hz, 3H), 2.77 (t,J = 13.1 Hz, 1H), 2.62-2.36 (m, 2H), 1.83- 1.58 (m, 2H). |
| 58 | 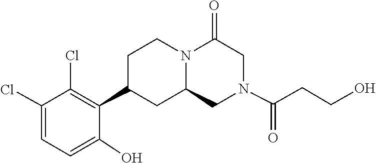 | (8S,9aR)-8-(2,3-dichloro-6- hydroxyphenyl)-2-(3-hydroxy- propanoyl)octahydro-4H- pyrido[1,2-a]pyrazin-4-one | [M + H]⁺: 387, 389 (3:2); ¹H NMR (400 MHz, CD₃OD) δ 7.20 (d, J = 8.8 Hz, 1H), 6.71 (d, J = 8.8 Hz, 1H), 4.78-4.67 (m, 1H), 4.62-4.35 (m, 1H), 4.32-4.15 (m, 1H), 4.16-4.01 (m, 1H), 3.85 (t, J = 6.3 Hz, 2H), 3.80-3.52 (m, 3H), 2.82-2.54 (m, 3H), 2.52-2.33 (m, 2H), 1.82-1.58 (m, 2H) |
| 59 | 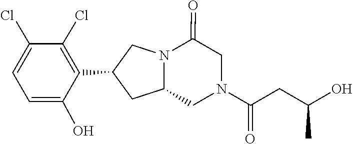 [SMILES: C[C@H](O)CC(=O)N1CC(=O)N2C[C@@H](c3c(O)ccc(Cl)c3Cl)C[C@H]2C1] | (7R,8aS)-7-(2,3-dichloro-6- hydroxyphenyl)-2-[(3S)-3- hydroxybutanoyl]- hexahydropyrrolo[1,2- a]pyrazin-4-one | [M + H]⁺: 387, 389 (3:2); ¹H NMR (400 MHz, CD₃OD) δ 7.27 (d, J = 8.8 Hz, 1H), 6.77 (d, J = 8.8 Hz, 1H), 4.68-4.50 (m, 1H), 4.48-4.12 (m, 3H), 4.12-3.69 (m, 2H), 3.65-3.53 (m, 1H), 3.27-3.11 (m, 1H), 2.80- 2.61 (m, 2H), 2.60-2.35 (m, 2H), 2.24-2.16 (m, 1H), 1.27 (d, J = 6.3 Hz, 3H). |

### Example 22. Compound 61 ((2R,8aS)-2-(2,3-dichloro-6-hydroxyphenyl)-7-hydroxyhexahydroindolizin-5(1H)-one isomer 1) and Compound 62 ((2R,8aS)-2-(2,3-dichloro-6-hydroxyphenyl)-7-hydroxyhexahydroindolizin-5(1H)-one isomer 2)

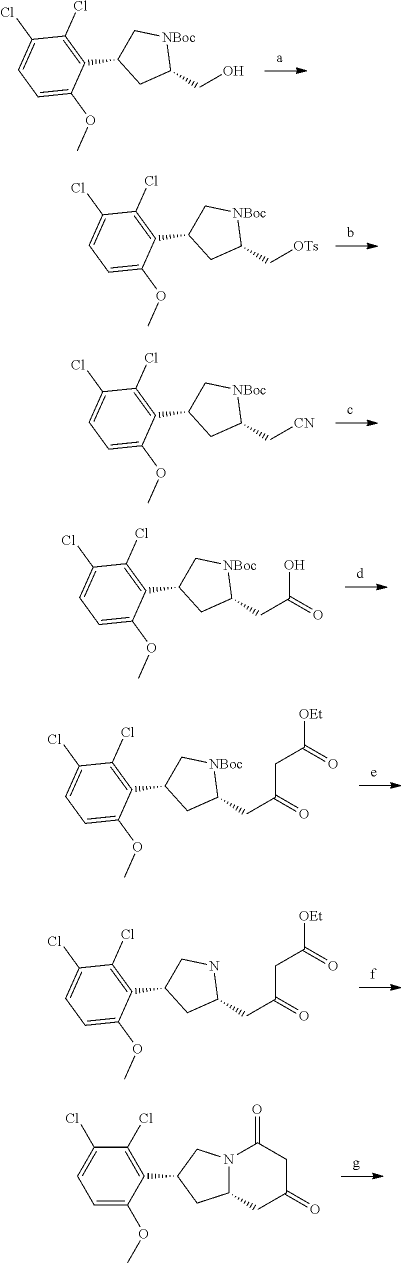

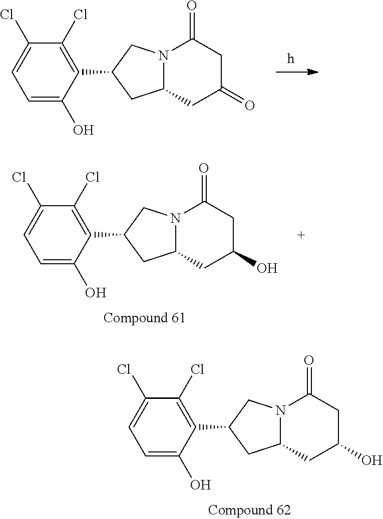 [SMILES: O=C1CC(=O)N2C[C@@H](c3c(O)ccc(Cl)c3Cl)C[C@H]2C1 ; O=C1C[C@@H](O)C[C@@H]2C[C@H](c3c(O)ccc(Cl)c3Cl)CN12 ; O=C1C[C@H](O)C[C@@H]2C[C@H](c3c(O)ccc(Cl)c3Cl)CN12]

Step a:

To a stirred solution of tert-butyl (2S,4R)-4-(2,3-dichloro-6-methoxyphenyl)-2-(hydroxymethyl)pyrrolidine-1-carboxylate (Example 7, step b) (6.6 g, 17.541 mmol, 1.00 equiv), TsCl (3.68 g, 19.295 mmol, 1.10 equiv) and DMAP (214 mg, 1.754 mmol, 0.10 equiv) in DCM (60 mL) was added TEA (3.55 g, 35.081 mmol, 2.00 equiv) at room temperature. The resulting mixture was stirred for 2 h at room temperature. The resulting mixture was diluted with water (50 mL). The resulting mixture was extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluted with PE/EA (3:1) to afford tert-butyl (2S,4R)-4-(2,3-dichloro-6-methoxyphenyl)-2-[[(4-methylbenzenesulfonyl)oxy]methyl]pyrrolidine-1-carboxylate (6.6 g, 64%): LCMS (ESI) calculated for C₂₄H₂₉Cl₂NO₆S [M+H]⁺: 530, 532 (3:2), found 530, 532 (3:2); ¹H NMR (300 MHz, Chloroform-d) δ 7.82 (d, J=8.2 Hz, 2H), 7.38-7.33 (m, 3H), 6.78 (d, J=9.0 Hz, 1H), 4.43-4.42 (m, 1H), 4.21-3.95 (m, 2H), 3.90-3.85 (m, 2H), 3.75-3.73 (m, 3H), 2.70-2.66 (m, 1H), 2.48-2.46 (m, 4H), 2.20-2.17 (m, 1H), 1.41 (s, 9H).

Step b:

To a stirred solution of tert-butyl (2S,4R)-4-(2,3-dichloro-6-methoxyphenyl)-2-[[(4-methylbenzenesulfonyl)oxy]methyl]pyrrolidine-1-carboxylate (1.0 g, 1.885 mmol, 1.00 equiv) in DMSO (10 mL) was added KCN (245 mg, 3.770 mmol, 2.00 equiv) at room temperature. The reaction was stirred at 80° C. for 1 h. The resulting mixture was diluted with NaHCO₃ (sat., 100 mL). The resulting mixture was extracted with EA (3×200 mL). The combined organic layers were washed with brine (3×200 mL) and dried over anhydrous Na₂SO₄. After filtration, the filtrate was concentrated under reduced pressure. The residue was purified by silica gel chromatography, eluted with PE/EA (3:1) to afford tert-butyl (2S,4R)-2-(cyanomethyl)-4-(2,3-dichloro-6-methoxyphenyl)pyrrolidine-1-carboxylate (3.4 g, 47%) as an off-white solid: LCMS (ESI) calculated for C₁₈H₂₂Cl₂N₂O₃ [M+H]⁺: 385, 387 (3:2), found 385, 387 (3:2); ¹H NMR (400 MHz, Methanol-d4) δ 7.44 (d, J=9.0 Hz, 1H), 7.02 (d, J=9.0 Hz, 1H), 4.17-4.04 (m, 2H), 3.92-3.86 (m, 4H), 3.71-3.63 (m, 1H), 3.19-3.15 (m, 1H), 2.88-0.67 (m, 2H), 2.33-2.31 (m, 1H), 1.53 (s, 9H).

Step c:

To a stirred solution of tert-butyl (2S,4R)-2-(cyanomethyl)-4-(2,3-dichloro-6-methoxyphenyl)pyrrolidine-1-carboxylate (2 g, 5.191 mmol, 1.00 equiv) in HCl (20 mL) was added AcOH (4 mL) at room temperature. The reaction was stirred at 100° C. for 1 h. The reaction was concentrated under reduced pressure. To the resulting residue was added DCM (20 mL), TEA (2.63 g, 25.991 mmol, 5.01 equiv), and Boc₂O (2.27 g, 10.382 mmol, 2.00 equiv), sequentially. The reaction mixture was stirred at room temperature for 1 h. The reaction mixture was concentrated under reduced pressure. The residue was purified by reverse phase chromatography, eluted with 65% ACN in water (plus 0.1% FA) to afford [(2S,4R)-1-(tert-butoxycarbonyl)-4-(2,3-dichloro-6-methoxyphenyl)pyrrolidin-2-yl]acetic acid (1.8 g, 86%) as an off-white solid: LCMS (ESI) calculated for C₁₈H₂₃Cl₂NO₅ [M+H]⁺: 404, 406 (3:2), found 404, 406 (3:2); ¹H NMR (400 MHz, Methanol-d4) δ 7.42 (d, J=9.0 Hz, 1H), 6.99 (d, J=9.0 Hz, 1H), 4.29-4.02 (m, 2H), 3.90 (s, 3H), 3.84-3.59 (m, 2H), 3.14-2.94 (m, 1H), 2.67-2.30 (m, 3H), 1.51 (s, 9H).

Step d:

To a stirred solution of [(2S,4R)-1-(tert-butoxycarbonyl)-4-(2,3-dichloro-6-methoxyphenyl)pyrrolidin-2-yl]acetic acid (1.1 g, 2.721 mmol, 1.00 equiv) in DCM (10 mL) and Meldrum's acid (0.59 g, 4.081 mmol, 1.50 equiv) were added DMAP (0.66 g, 5.442 mmol, 2.00 equiv) and EDCI (0.78 g, 4.081 mmol, 1.50 equiv) at room temperature. The reaction was stirred at room temperature for 1 h. The resulting solution was concentrated under reduced pressure. The residue was dissolved in EtOH (10 mL) and the resulting mixture was stirred at 90° C. for 16 h. Then, TsOH (243 mg, 1.36 mmol, 0.50 equiv) was added. The reaction mixture was stirred at 100° C. for 16 h. The resulting mixture was quenched with water (40 mL) and extracted with EA (3×50 mL). The combined organic layers were washed with brine (3×50 mL) and dried over anhydrous Na₂SO₄. After filtration, the filtrate was concentrated under reduced pressure. The residue was purified by reverse phase chromatography, eluted with 65% ACN in water (plus 0.05% TFA) to afford tert-butyl (2S,4R)-4-(2,3-dichloro-6-methoxyphenyl)-2-(4-ethoxy-2,4-dioxobutyl)pyrrolidine-1-carboxylate (1 g, 77%) as a yellow oil: LCMS (ESI) calculated for C₂₂H₂₉Cl₂NO₆ [M+H]⁺: 474, 476 (3:2), found 474, 476 (3:2); ¹H NMR (400 MHz, Chloroform-d) δ 7.33 (d, J=8.9 Hz, 1H), 6.75 (d, J=9.0 Hz, 1H), 4.29-3.99 (m, 4H), 3.85 (s, 3H), 3.81-3.57 (m, 2H), 3.51-3.41 (m, 3H), 2.85-2.79 (m, 1H), 2.49-2.15 (m, 2H), 1.49 (s, 9H), 1.31-1.28 (m, 3H).

Step e:

To a stirred solution of tert-butyl (2S,4R)-4-(2,3-dichloro-6-methoxyphenyl)-2-(4-ethoxy-2,4-dioxobutyl)pyrrolidine-1-carboxylate (300 mg, 0.632 mmol, 1.00 equiv) in DCM (3 mL) was added TFA (1.5 mL) at room temperature. The resulting mixture was stirred for 1 h at room temperature. The resulting mixture was concentrated under vacuum to afford ethyl 4-[(2S,4R)-4-(2,3-dichloro-6-methoxyphenyl)pyrrolidin-2-yl]-3-oxobutanoate (300 mg, crude) as a yellow oil: LCMS (ESI) calculated for C₁₇H₂₁Cl₂NO₄ [M+H]⁺: 374, 376 (3:2), found 374, 376 (3:2).

Step f:

To a stirred solution of ethyl 4-[(2S,4R)-4-(2,3-dichloro-6-methoxyphenyl)pyrrolidin-2-yl]-3-oxobutanoate (300 mg, 0.802 mmol, 1.00 equiv) in MeOH (3 mL) were added LiOH.H₂O (67 mg, 1.603 mmol, 2.00 equiv) and H₂O (1.5 mL). The resulting mixture was stirred for 1 h at room temperature. The resulting mixture was concentrated under vacuum. The residue was purified by reverse phase chromatography, eluted with 50% ACN in in water (plus 0.05% TFA) to afford (2R,8aS)-2-(2,3-dichloro-6-methoxyphenyl)-hexahydroindolizine-5,7-dione (90 mg, 34%) as a yellow oil: LCMS (ESI) calculated for C₁₅H₁₅Cl₂NO₃ [M+H]⁺: 328, 330 (3:2), found 328, 330 (3:2); ¹H NMR (400 MHz, Chloroform-d) δ 7.38 (dd, J=8.9, 2.4 Hz, 1H), 6.80 (dd, J=9.0, 3.4 Hz, 1H), 4.30-4.05 (m, 2H), 3.85-3.83 (m, 4H), 3.40-3.23 (m, 2H), 2.90-2.61 (m, 2H), 2.58-2.28 (m, 2H), 2.13-2.10 (m, 1H).

Step g:

To a stirred solution of (2R,8aS)-2-(2,3-dichloro-6-methoxyphenyl)-hexahydroindolizine-5,7-dione (300 mg, 0.609 mmol, 1.00 equiv) in DCM (1.00 mL) was added BBr₃ (0.9 mL, 10 equiv) at room temperature. The resulting mixture was stirred for 2 h at room temperature. The reaction was quenched with water (2 mL). The resulting mixture was concentrated under vacuum. The residue was purified by reverse phase chromatography, eluted with 20% ACN in water (plus 10 mmol/L NH₄HCO₃) to afford (2R,8aS)-2-(2,3-dichloro-6-hydroxyphenyl)-hexahydroindolizine-5,7-dione (180 mg, 58%) as an off-white solid: LCMS (ESI) calculated for C₁₄H₁₃Cl₂NO₃ [M+H]⁺: 314, 316 (3:2), found 314, 316 (3:2); ¹H NMR (400 MHz, Methanol-d4) δ 7.29-7.24 (m, 1H), 6.79-6.74 (m, 1H), 4.61 (s, 2H), 4.50-4.04 (m, 3H), 3.91-3.71 (m, 1H), 2.86-2.74 (m, 1H), 2.63-2.30 (m, 2H), 2.18-2.06 (m, 1H).

Step h:

To a stirred solution of (2R,8aS)-2-(2,3-dichloro-6-hydroxyphenyl)-hexahydroindolizine-5,7-dione (180 mg, 0.516 mmol, 1.00 equiv, 90%) in THE (2 mL) was added NaBH₄ (39 mg, 1.026 mmol, 1.99 equiv) at room temperature. The resulting mixture was stirred for 1 h at room temperature. The resulting mixture was diluted with water (3 mL). The resulting mixture was extracted with EA (3×10 mL). The combined organic layers were washed with brine (3×5 mL) and dried over anhydrous Na₂SO₄. After filtration, the filtrate was concentrated under reduced pressure. The residue was purified by CHIRAL-HPLC with the following conditions (Column: CHIRALPAK IE, 2*25 cm, 5 um; Mobile Phase A: Hex (0.1% FA), Mobile Phase B: EtOH; Flow rate: 20 mL/min; Gradient: 20 B to 20 B in 11 min; 220/254 nm. Retention time: 6.98 min and 8.68 min. The faster-eluting isomer was obtained as Compound 61 ((2R,8aS)-2-(2,3-dichloro-6-hydroxyphenyl)-7-hydroxy-hexahydro-1H-indolizin-5-one isomer 1) (10 mg, 2%) as an off-white solid: LCMS (ESI) calculated for C₁₄H₁₅Cl₂NO₃ [M+H]⁺: 316, 318 (3:2), found 316, 318 (3:2); ¹H NMR (400 MHz, Methanol-d4) δ 7.25 (d, J=8.8 Hz, 1H), 6.78 (d, J=8.8 Hz, 1H), 4.20-4.05 (m, 4H), 3.57-3.52 (m, 1H), 2.76-2.70 (m, 1H), 2.47-2.34 (m, 2H), 2.28-2.21 (m, 1H), 2.11-2.02 (m, 1H), 1.52-1.43 (m, 1H).

The slower-eluting isomer was obtained as Compound 62 ((2R,8aS)-2-(2,3-dichloro-6-hydroxyphenyl)-7-hydroxy-hexahydro-1H-indolizin-5-one isomer 2) (45 mg, 43%) as an off-white solid: LCMS (ESI) calculated for C₁₄H₁₅Cl₂NO₃ [M+H]⁺: 316, 318 (3:2), found 316, 318 (3:2); ¹H NMR (400 MHz, Methanol-d4) δ 7.25 (d, J=8.8 Hz, 1H), 6.75 (d, J=8.8 Hz, 1H), 4.29-4.21 (m, 1H), 4.13-4.06 (m, 2H), 3.78-3.72 (m, 1H), 3.52-3.46 (m, 1H), 2.79-2.73 (m, 1H), 2.44-2.35 (m, 2H), 2.28-2.13 (m, 2H), 1.53-1.44 (m, 1H).

Example 23. Compounds 63-64 were prepared in an analogous fashion to an example disclosed herein and/or analogous to known methods in the art.

| Compound | | | |
|---|---|---|---|
| Number | Structure | Chemical Name | MS: (M + H)⁺ & ¹H MNR |
| 63 | 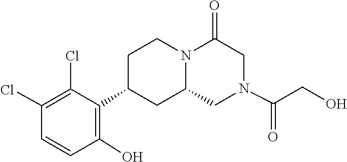 [SMILES: O=C(CO)N1CC(=O)N2CC[C@@H](c3c(O)ccc(Cl)c3Cl)C[C@H]2C1] | (8R,9aS)-8-(2,3- dichloro-6- hydroxyphenyl)- 2-(2- hydroxyacetyl)- hexahydro-1H- pyrido[1,2- a]pyrazin-4-one | [M + H]⁺: 373, 375 (3:2); ¹H NMR (400 MHz, DMSO-d₆ + D₂O) δ 7.31 (d, J = 8.8 Hz, 1H), 6.81 (d, J =8.8 Hz, 1H), 4.59-4.52 (m, 1H), 4.24-4.05 (m, 3H), 4.03-3.93 (m, 1H), 3.91-3.82 (m, 1H), 3.64-3.53 (m, 2H), 3.43-3.32 (m, 1H), 2.64 (td, J = 13.8 Hz, 3.2 Hz, 1H), 2.30-2.10 (m, 2H), 1.69-1.45 (m, 2H). |
| 64 | 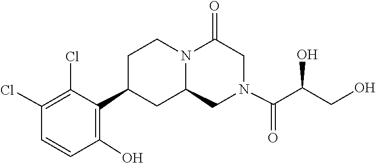 [SMILES: O=C([C@@H](O)CO)N1CC(=O)N2CC[C@H](c3c(O)ccc(Cl)c3Cl)C[C@@H]2C1] | (8S,9aR)-8-(2,3- dichloro-6- hydroxyphenyl)- 2-[(2S)-2,3- dihydroxy- propanoyl]- hexahydro-1H- pyrido[1,2- a]pyrazin-4-one | [M + H]⁺: 403, 405 (3:2); ¹H NMR (400 MHz, CD₃OD) δ 7.20 (d, J = 8.7 Hz, 1H),6.71 (d, J = 8.8 Hz, 1H), 4.78-4.71 (m, 1H), 4.57-3.99 (m, 4H), 3.85-3.50 (m, 5H), 2.81-2.70 (m, 1H), 2.58-2.30 (m, 2H), 1.81-1.57 (m, 2H). |

Example 24. Compound 65-78 were prepared in an analogous fashion to an example disclosed herein and/or analogous to known methods in the art.

| Compound | | | |
|---|---|---|---|
| Number | Structure | Chemical Name | MS: (M + H)⁺ & ¹H MNR |
| 65 | 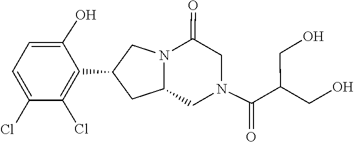 [SMILES: O=C(C(CO)CO)N1CC(=O)N2C[C@@H](c3c(O)ccc(Cl)c3Cl)C[C@H]2C1] | (7R,8aS)-7-(2,3- dichloro-6- hydroxyphenyl)- 2-[3-hydroxy-2- (hydroxymethyl) propanoyl]-hexa- hydropyrrolo[1,2- a]pyrazin-4-one | [M + H]⁺: 403,405 (3:2); ¹H NMR (400 MHz, CD₃OD) δ 7.27 (d, J = 8.8 Hz, 1H), 6.77 (d, J = 8.8 Hz, 1H), 5.00-4.95 (m, 1H), 4.76- 4.59 (m, 2H), 4.36-4.31 (m, 1H), 4.21- 4.08 (m, 1H), 4.02-3.98 (m, 1H), 3.86- 3.64 (m, 5H), 3.59 (t J = 10.6 Hz, 1H), 3.22 (dd, J = 13.4, 10.2 Hz, 1H), 2.51- 2.38 (m, 1H), 2.23-2.20 (m, 1H). |
| 66 | 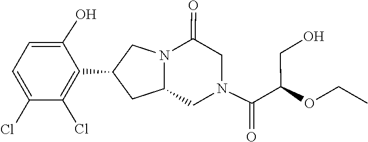 [SMILES: CCO[C@H](CO)C(=O)N1CC(=O)N2C[C@@H](c3c(O)ccc(Cl)c3Cl)C[C@H]2C1] | (7R,8aS)-7-(2,3- dichloro-6- hydroxyphenyl)- 2-[(2R)-2- ethoxy-3- hydroxypropanoyl]- hexahydropyrrolo[1,2-a]pyrazin- 4-one | [M + H]⁺: 417, 419(3:2); ¹H NMR (400 MHz, CD₃OD) δ 7.27 (d, J = 8.8 Hz, 1H), 6.77 (d, J = 8.7 Hz, 1H), 4.98-4.60 (m, 2H), 4.24-4.14 (m, 2H), 4.09 (dd, J = 17.5, 6.7 Hz, 1H), 4.02-3.96 (m, 1H), 3.83-3.75 (m, 3H), 3.65-3.53 (m, 3H), 3.24-3.16 (m, 0.5 H), 2.82 (t, J = 11.8 Hz, 0.5H), 2.47-2.45 (m, 1H), 2.11-2.19 (m, 1H), |
| | | | 1.33-1.20 (m, 3H). |
| 67 | 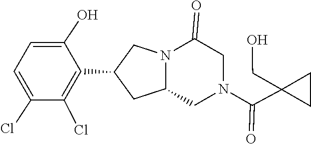 [SMILES: O=C1CN(C(=O)C2(CO)CC2)C[C@@H]2C[C@H](c3c(O)ccc(Cl)c3Cl)CN12] | (7R,8aS)-7-(2,3- dichloro-6- hydroxyphenyl)-2-[1-(hydroxymethyl) cyclopro- panecarbonyl]- hexahydropyrrolo[1,2-a]pyrazin- 4-one | [M + H]⁺:399, 401 (3:2); ¹H NMR (400 MHz, CD₃OD) δ 7.27 (d, J = 8.8 Hz, 1H), 6.77 (d, J = 8.8 Hz, 1H), 4.89-4.60 (m, 1H), 4.38-4.27 (m, 1H), 4.24-4.14 (m, 1H), 3.97-3.93 (m, 2H), 3.60-3.56 (m, 4H), 3.20-2.90 (m, 1H), 2.46-2.42 (m, 1H), 2.25-2.14 (m, 1H), 1.05-0.80 (m, 4H). |
| 68 | 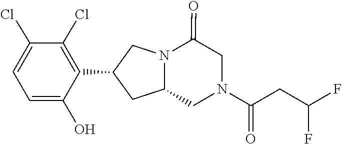 [SMILES: O=C(CC(F)F)N1CC(=O)N2C[C@@H](c3c(O)ccc(Cl)c3Cl)C[C@H]2C1] | (7R,8aS)-7-(2,3- dichloro-6- hydroxyphenyl)- 2-(3,3- difluoropropanoyl)- hexahydropyrrolo [1,2-a]pyrazin-4- one | [M + H]⁺: 393, 395 (3:2); ¹H NMR (400 MHz, CD₃OD) δ 7.27 (d, J = 8.8 Hz, 1H), 6.77 (d, J = 8.8 Hz, 1H), 6.51-6.05 (m, 1H), 4.63 (d, J = 18.4 Hz, 1H), 4.43-4.27 (m, 2H), 4.18 (t, J = 10.4 Hz, 1H), 4.06-3.75 (m, 2H), 3.60 (t, J = 10.5 Hz, 1H), 3.25-3.05 (m, 2.5H), 2.81-2.70 (m, 0.5H), 2.47-2.43 (m, 1H), 2.23-2.19 (m, 1H); |
| 69 | 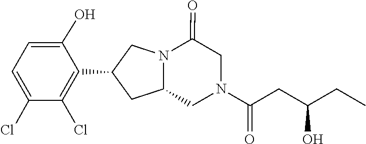 [SMILES: CC[C@@H](O)CC(=O)N1CC(=O)N2C[C@@H](c3c(O)ccc(Cl)c3Cl)C[C@H]2C1] | (7R,8aS)-7-(2,3-dichloro-6- hydroxyphenyl)- 2-[(3R)-3- hydroxypentanoyl]- hexahydropyrrolo [1,2-a]pyrazin-4- one | [M + H]⁺: 401, 403 (3:2); ¹H NMR (400 MHz, CD₃OD) δ 7.27 (d, J = 8.8 Hz, 1H), 6.77 (d, J = 8.8 Hz, 1H), 4.68-4.46 (m, 2H), 4.41-4.25 (m, 1H), 4.23-4.14 (m, 1H), 4.09-3.73 (m, 3H), 3.59 (td, J = 10.8, 4.4 Hz, 1H), 3.23-3.15 (m, 0.5H), 2.79-2.72 (m, 0.5H), 2.72-2.23 (m, 3H), 2.21-2.17 (m, 1H), 1.67-1.46 (m, 2H), 1.00 (t, J = |
| | | | 7.4 Hz, 3H). |
| 70 | 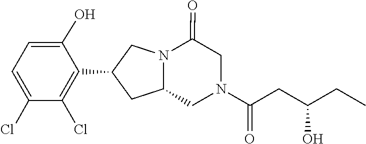 [SMILES: CC[C@H](O)CC(=O)N1CC(=O)N2C[C@@H](c3c(O)ccc(Cl)c3Cl)C[C@H]2C1] | (7R,8aS)-7-(2,3- dichloro-6- hydroxyphenyl)- 2-[(3S)-3- hydroxypentanoyl]- hexahydropyrrolo [1,2-a]pyrazin-4- one | [M + H]⁺: 401,403 (3:2); ¹H NMR (400 MHz, CD₃OD) δ 7.27 (d, J = 8.8 Hz, 1H), 6.77 (d, J = 8.8 Hz, 1H), 4.73-4.60 (m, 1H), 4.51-4.43 (m, 1H), 4.35-4.30 (m, 1H), 4.23-4.14 (m, 1H), 4.09-3.73 (m, 3H), 3.59 (td, J = 10.8, 4.4 Hz, 1H), 3.23-3.15 (m, 0.5H), 2.79-2.72 (m, 0.5H), 2.72-2.23 (m, 3H), 2.21-2.17 (m, 1H), 1.67-1.46 (m, 2H), |
| | | | 1.00 (t, J = 7.4 Hz, 3H). |
| 71 | 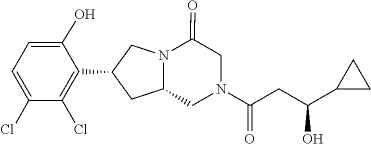 [SMILES: O=C(C[C@H](O)C1CC1)N1CC(=O)N2C[C@@H](c3c(O)ccc(Cl)c3Cl)C[C@H]2C1] | (7R,8aS)-2-[(3S)- 3-cyclopropyl-3- hydroxypropanoyl]- 7-(2,3-dichloro-6- hydroxyphenyl)- hexahydropyrrolo [1,2-a]pyrazin-4- one | [M+ H]⁺: 413, 415 (3:2); ¹H NMR (400 MHz, CD₃OD) δ 7.77 (d, J = 8.8 Hz, 1H), 6.77 (d, J = 8.8 Hz, 1H), 4.95-4.92 (m, 0.5H), 4.68-4.51 (m, 1.5H), 4.42-4.25 (m, 1H), 4.23-4.14 (m, 1H), 4.10-3.72 (m, 2H), 3.59 (td, J = 10.5, 4.7 Hz, 1H), 3.42-3.34 (m, 0.5H), 3.23-3.15 (m, 0.5H), 2.87-2.58 (m, 2H), 2.48-2.40 (m, 1H), 2.25-2.17 (m, 1H), |
| | | | 1.04-0.94 (m, 1H), 0.61-0.47 (m, 2H), |
| | | | 0.44-0.38 (m, 1H), 0.30-0.24 (m, 1H). |
| 72 | 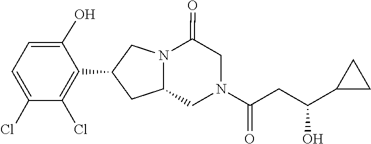 [SMILES: O=C(C[C@@H](O)C1CC1)N1CC(=O)N2C[C@@H](c3c(O)ccc(Cl)c3Cl)C[C@H]2C1] | (7R,8aS)-2-[(3R)-3- cyclopropyl-3- hydroxypropanoyl]- 7-(2,3-dichloro-6- hydroxyphenyl)- hexahydropyrrolo [1,2-a]pyrazin-4- one | [M+ H]⁺: 413, 415 (3:2); ¹H NMR (400 MHz, CD₃OD) δ 7.77 (d, J = 8.8 Hz, 1H), 6.77 (d, J = 8.8 Hz, 1H), 4.95-4.92 (m, 0.5H), 4.73-4.51(m, 1.5H), 4.39-4.29 (m, 1H),4.23-4.14 (m,1H), 4.10-3.72 (m, 2H), 3.59 (td, J = 10.5, 4.7 Hz, 1H), 3.42-3.34 (m, 0.5H), 3.23-3.15 (m, 0.5H), 2.79-2.61 (m, 2H), 2.48-2.40 (m, 1H), 2.25-2.17 (m, 1H), |
| | | | 1.04-0.94 (m, 1H), 0.61-0.46 (m, 2H), |
| | | | 0.44-0.38 (m, 1H), 0.33-0.22 (m, 1H). |
| 73 | 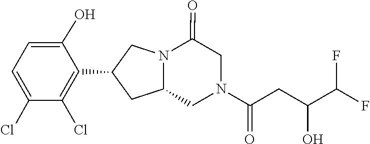 [SMILES: O=C(CC(O)C(F)F)N1CC(=O)N2C[C@@H](c3c(O)ccc(Cl)c3Cl)C[C@H]2C1] | (7R,8aS)-7-(2,3- dichloro-6- hydroxyphenyl)- 2-(4,4-difluoro-3- hydroxybutanoyl)- hexahydropyrrolo [1,2-a]pyrazin-4- one | [M + H]⁺_{:} 423, 425 (3:2); ¹H NMR (400 MHz, CD₃OD) δ 7.27 (d, J = 9.0 Hz, 1H), 6.77 (d, J = 8.8 Hz, 1H), 5.85 (tt, J =56.0, 3.2 Hz, 1H), 4.97- 4.95 (m, 0.5H), 4.72-4.65 (m, 0.5H), 4.53-4.40 (m, 1H), 4.40-4.16 (m, 3H), 4.12-3.73 (m, 2H), 3.59 (td, J = 10.5, 4.7 Hz, 1H), 3.25-3.15 (m, 0.5H), 2.90- 2.53 (m, 2.5H), 2.49-2.41 (m, 1H), 2.25- |
| | | | 2.17 (m, 1H); ¹⁹F NMR (376 MHz, |
| | | | CD₃OD) δ-131.47 (s, 2F). |
| 74 | 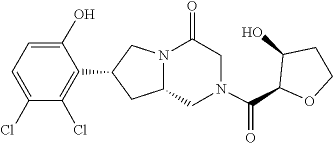 [SMILES: O=C([C@@H]1OCC[C@@H]1O)N1CC(=O)N2C[C@@H](c3c(O)ccc(Cl)c3Cl)C[C@H]2C1] | (7R,8aS)-7-(2,3- dichloro-6- hydroxyphenyl)- 2-[(2R,3S)-3- hydroxyoxolane- 2-carbonyl]- hexahydropyrrolo [1,2-a]pyrazin-4-one | [M + H]⁺: 415, 417 (3:2); ¹H NMR (400 MHz, CD₃OD) δ 7.27 (d, J = 8.9 Hz, 1H), 6.77 (d, J = 8.8 Hz, 1H), 4.83-4.70 (m, 2H), 4.65 (d, J = 18.6 Hz, 1H), 4.55 (t, J = 17.3 Hz, 1H), 4.41-4.25 (m, 1H), 4.23-4.11 (m, 2H), 4.09-3.71 (m, 3H), 3.62-3.53 (m, 1H), 3.17-3.11 (m, 0.5H), 2.85-2.74 (m, 0.5H), 2.48-2.38 (m, 1H), 2.24-2.15 |
| | | | (m, 2H), 2.02-1.95 (m, 1H). |
| 75 | 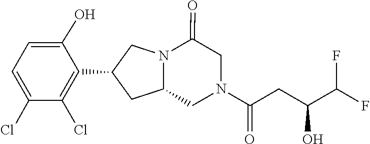 [SMILES: O=C(C[C@H](O)C(F)F)N1CC(=O)N2C[C@@H](c3c(O)ccc(Cl)c3Cl)C[C@H]2C1] | (7R,8aS)-7-(2,3- dichloro-6- hydroxyphenyl)- 2-[(3S)-4,4- difluoro-3- hydroxybutanoyl] hexahydropyrrolo [1,2-a]pyrazin-4-one | [M + H]⁺: 423, 425 (3:2); ¹H NMR (400 MHz, CD₃OD) δ 7.27 (d, J = 8.8 Hz, 1H), 6.77 (d, J = 8.8 Hz, 1H), 5.85 (tt, J = 55.9, 3.5 Hz, 1H), 4.69 (d, J = 18.5 Hz, 1H), 4.50-4.40 (m, 1H), 4.39-4.16 (m, 3H), 4.12-3.72 (m, 2H), 3.59 (td, J = 11.3, 9.7 Hz, 1H), 3.23-3.15 (0.5H), 2.91-2.67 (m, 2H), 2.63 (dd, J = 15.5, 3.5 Hz, 0.5H), 2.49-2.41 (m, 1H), |
| | | | 2.25-2.17 (m, 1H); ¹⁹F NMR (376 MHz, |
| | | | CD₃OD) δ-131.44 (s, 2F). |
| 76 | 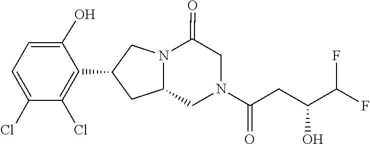 [SMILES: O=C(C[C@@H](O)C(F)F)N1CC(=O)N2C[C@@H](c3c(O)ccc(Cl)c3Cl)C[C@H]2C1] | (7R,8aS)-7-(2,3- dichloro-6- hydroxyphenyl)- 2-[(3R)-4,4- difluoro-3- hydroxybutanoyl]- hexahydropyrrolo [1,2-a]pyrazin-4-one | [M + H]⁺: 423,425 (3:2); ¹H NMR (400 MHz, CD₃OD) δ 7.27 (d, J = 8.8 Hz, 1H), 6.79 (d, J = 8.8 Hz, 1H), 5.88 (tt, J =55.6, 2.8 Hz, 1H), 4.62 (d, J = 18.5 Hz, 1H), 4.55-4.36 (m, 1H), 4.36-4.19 (m, 2H), 4.15-4.11 (m, 1H), 4.08-3.76 (m, 2H), 3.59 (td, J = 11.3, 9.7 Hz, 1H), 3.24-3.19 (0.5H), 2.84-2.71 (m, 2H),2.63 (dd, J = 15.8, 3.8 Hz, 0.5H), |
| | | | 2.43-2.33 (m, 1H), 2.26-2.18 (m, 1H); |
| | | | ¹⁹F NMR (376 MHz, CD₃OD) δ-131.19 |
| | | | (s, 2F). |
| 77 | 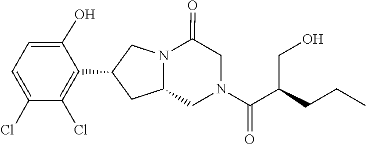 [SMILES: CCC[C@H](CO)C(=O)N1CC(=O)N2C[C@@H](c3c(O)ccc(Cl)c3Cl)C[C@H]2C1] | (7R,8aS)-7-(2,3- dichloro-6- hydroxyphenyl)- 2-[(2R)-2- (hydroxymethyl) pentanoyl]- hexahydropyrrolo [1,2-a]pyrazin-4-one | [M + H]⁺: 415, 417(3:2); ¹H NMR (400 MHz, CD₃OD) δ 7.27 (d, J = 8.9 Hz, 1H), 6.77 (d, J = 8.8 Hz, 1H), 5.01-4.92 (m, 0.5H),4.74-4.61 (m, 1.5H), 4.41-4.25 (m, 1H), 4.24-4.14 (m, 1H), 4.10-3.77 (m, 2H), 3.75-3.54 (m, 3H), 3.22-3.12 (m, 1H), 3.09-3.02 (m, 0.5H), 2.77(dd, J = 13.0, 10.6 Hz, 0.5H), 2.49-2.40 (m, 1H), 2.27-2.17 (m, |
| | | | 1H), 1.66-1.54 (m, 1H), 1.49-1.30 |
| | | | (m, 3H), 0.95 (td, J = 7.1, 4.9 Hz, 3H). |
| 78 | 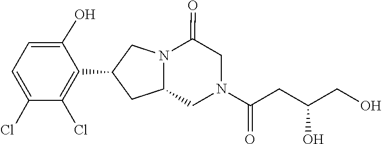 [SMILES: O=C(C[C@@H](O)CO)N1CC(=O)N2C[C@@H](c3c(O)ccc(Cl)c3Cl)C[C@H]2C1] | (7R,8aS)-7-(2,3- dichloro-6- hydroxyphenyl)- 2-[(3R)-3,4- dihydroxybutanoyl]- hexahydropyrrolo [1,2-a]pyrazin-4- one | [M + H]⁺: 403,405 (3:2); ¹H NMR (400 MHz CD₃0D) δ 7.27, (d, J = 8.8 Hz, 1H), 6.77 (d, J = 8.8 Hz, 1H), 4.95-4.93 (m, 0.5H), 4.71 (d, J = 18.5 Hz, 0.5H), 4.53-4.44 (m, 1H), 4.38- 4.28 (m, 1H), 4.24-4.12 (m, 1H), 4.15- 3.70 (m, 3H), 3.64-3.53 (m, 3H), 3.23- 3.15 (m, 0.5H), 2.79-2.52(m, 2.5H), 2.48-2.40 (m, 1H), 2.26-2.14 (m, 1H). |

### Example 25. Compound 57 ((3R,8R,9aS)-8-(2,3-dichloro-6-hydroxyphenyl)-3-(hydroxymethyl)-octahydropyrido[1,2-a]pyrazin-4-one) and Compound 36 (3R,8S,9aR)-8-(2,3-dichloro-6-hydroxyphenyl)-3-(hydroxymethyl)-octahydropyrido[1,2-a]pyrazin-4-one

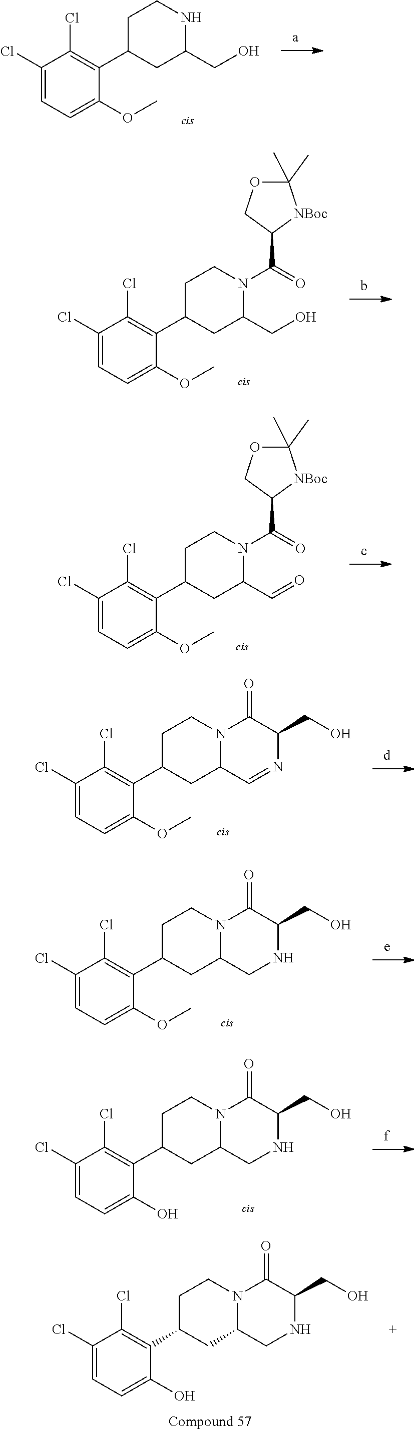

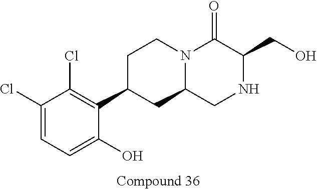 [SMILES: O=C1[C@@H](CO)NC[C@H]2C[C@@H](c3c(O)ccc(Cl)c3Cl)CCN12]

Step a:

To a stirred solution of (4R)-3-(tert-butoxycarbonyl)-2,2-dimethyl-1,3-oxazolidine-4-carboxylic acid (0.180 g, 0.74 mmol) and HATU (0.280 g, 0.74 mmol) in DMF (2 mL) were added [4-(2,3-dichloro-6-methoxyphenyl)piperidin-2-yl]methanol (cis, racemic) (0.190 g, 0.67 mmol) and TEA (0.140 g, 1.34 mmol) at room temperature. The reaction was stirred for 0.5 h and the resulting mixture purified directly by reverse phase chromatography, eluting with 55% ACN in 0.05% TFA aqueous solution to afford tert-butyl (4R)-4-[4-(2,3-dichloro-6-methoxyphenyl)-2-(hydroxymethyl)piperidine-1-carbonyl]-2,2-dimethyl-1,3-oxazolidine-3-carboxylate (cis, a mixture of two diasteroisomers) (0.230 g, 71%) as a light yellow solid. LCMS (ESI) calc'd for C₁₅H₁₈Cl₂N₂O₃ [M+H]⁺: 517, 519 (3:2) found 517, 519 (3:2); ¹H NMR (400 MHz, CDCl₃) δ 7.36-7.29 (m, 1H), 6.80-6.72 (m, 1H), 4.94-4.64 (m, 1H), 4.56-4.12 (m, 3H), 4.12-3.90 (m, 1H), 3.90-3.72 (m, 4H), 3.72-3.30 (m, 2H), 3.10-2.97 (m, 1H), 2.64-2.28 (m, 1H), 2.24-1.95 (m, 3H), 1.79-1.65 (m, 3H), 1.65-1.53 (m, 3H), 1.52-1.47 (m, 9H).

Step b:

To a stirred solution of tert-butyl (4R)-4-[4-(2,3-dichloro-6-methoxyphenyl)-2-(hydroxymethyl)piperidine-1-carbonyl]-2,2-dimethyl-1,3-oxazolidine-3-carboxylate (0.230 g, 0.44 mmol) in DCM (1.00 mL) was added Dess-Martin periodinane (0.280 g, 0.67 mmol) at room temperature. The reaction was stirred for 1 h, quenched with aq. Na₂SO₃ (1 mL), diluted with water (20 mL) and extracted with EA (2×30 mL). The combined organic layers were washed with brine (2×20 mL) and dried over anhydrous Na₂SO₄. After filtration, the filtrate was concentrated under reduced pressure. The residue was purified by reverse phase chromatography, eluting with 70% ACN in 0.05% TFA aqueous solution to afford tert-butyl (4R)-4-[4-(2,3-dichloro-6-methoxyphenyl)-2-formylpiperidine-1-carbonyl]-2,2-dimethyl-1,3-oxazolidine-3-carboxylate (cis, a mixture of two isomers) as a yellow oil (0.150 g, 65%). LCMS (ESI) calc'd for C₂₄H₃₂Cl₂N₂O₆ [M+1]⁺ 515, 517 (3:2) found 515, 517 (3:2); ¹H NMR (400 MHz, CDCl₃) δ 9.55 (d, J=25.0 Hz, 1H), 7.42-7.30 (m, 1H), 6.82-6.73 (m, 1H), 4.99-4.79 (m, 1H), 4.58-4.41 (m, 1H), 4.38-4.19 (m, 2H), 4.19-3.93 (m, 1H), 3.94-3.65 (m, 4H), 3.63-3.26 (m, 1H), 2.66-2.32 (m, 1H), 2.35-1.76 (m, 3H), 1.79-1.64 (m, 3H), 1.63-1.40 (m, 12H).

Step c:

To a stirred solution of tert-butyl (4R)-4-[4-(2,3-dichloro-6-methoxyphenyl)-2-(dihydroxymethyl)piperidine-1-carbonyl]-2,2-dimethyl-1,3-oxazolidine-3-carboxylate (0.150 g, 0.29 mmol) in DCM (2 mL) was added TFA (0.5 mL) at room temperature. The reaction was stirred for 2 h and concentrated under reduced pressure to afford 8-(2,3-dichloro-6-methoxyphenyl)-3-(hydroxymethyl)-1H,6H,7H,8H,9H,9aH-pyrido[1,2-a]pyrazin-4-one (cis, a mixture of two isomers) as a yellow oil (0.110 g, crude), which was used in the next step directly without purification: LCMS (ESI) calc'd for C₁₆H₁₈Cl₂N₂O₃ [M+1]⁺ 357, 359 (3:2) found 357, 359 (3:2).

Step d:

To a stirred solution of 8-(2,3-dichloro-6-methoxyphenyl)-3-(hydroxymethyl)-1H,6H,7H,8H,9H,9aH-pyrido[1,2-a]pyrazin-4-one (cis, a mixture of two isomers) (0.110 g, 0.31 mmol) in MeOH (2 mL) was added PtO₂ (20 mg) at room temperature. The reaction was stirred for 1 h under H₂ (1.5 atm). The reaction was filtered and the filtrate was concentrated under reduced pressure. The residue was purified by reverse phase chromatography, eluting with 26% ACN in 0.05% TFA aqueous solution to afford (3R)-8-(2,3-dichloro-6-methoxyphenyl)-3-(hydroxymethyl)-octahydropyrido[1,2-a]pyrazin-4-one (cis, a mixture of two isomers) as a yellow oil (80.0 mg, 72%): LCMS (ESI) calc'd for C₁₆H₂₀Cl₂N₂O₃ [M+1]⁺ 359, 361 (3:2) found 359, 361 (3:2).

Step e:

To a stirred solution of (3R)-8-(2,3-dichloro-6-methoxyphenyl)-3-(hydroxymethyl)-octahydropyrido[1,2-a]pyrazin-4-one (cis, a mixture of two isomers) (80.0 mg, 0.22 mmol) in DCM (2 mL) was added BBr₃ (0.560 g, 2.23 mmol) at room temperature. The reaction was stirred for 2 h, quenched with MeOH (2 mL) and concentrated under reduced pressure. The residue was purified by Prep-HPLC with the following conditions: Column: XBridge Prep C18 OBD Column, 5 um, 19×150 mm; Mobile Phase A: Water (plus 0.05% TFA), Mobile Phase B: ACN; Flow rate: 25 mL/min; Gradient: 28% B to 40% B in 7 min; Detector: UV: 254/220 nm; Retention Time: 6.30 min. The fractions containing the desired product were collected and concentrated under reduced pressure to afford (3R,8R,9aS)-8-(2,3-dichloro-6-hydroxyphenyl)-3-(hydroxymethyl)-octahydropyrido[1,2-a]pyrazin-4-one (cis, a mixture of two isomers) as an off-white solid (18.0 mg, 23%): LCMS (ESI) calc'd for C₁₅H₁₈Cl₂N₂O₃ [M+1]⁺ 345, 347 (3:2) found 345, 347 (3:2). ¹H NMR (400 MHz, CD₃OD) δ 7.23 (d, J=8.8 Hz, 1H), 6.73 (d, J=8.8 Hz, 1H), 4.84-4.67 (m, 1H), 4.21-3.96 (m, 3H), 3.93-3.68 (m, 2H), 3.68-3.47 (m, 1H), 3.39-3.35 (m, 1H), 2.90-2.72 (m, 1H), 2.62-2.34 (m, 2H), 1.91-1.61 (m, 2H).

Step f:

3R)-8-(2,3-dichloro-6-hydroxyphenyl)-3-(hydroxymethyl)-octahydropyrido[1,2-a]pyrazin-4-one (cis, mixture of two isomers) (12 mg, 0.04 mmol) was separated by Chiral Prep-HPLC with the following conditions: Column: CHIRALPAK IG, 2×25 cm, 5 μm; Mobile Phase A: Hex (plus 0.2% IPA)-HPLC, Mobile Phase B: EtOH-HPLC; Flow rate: 20 mL/min; Gradient: 20% B to 20% B in 14 min; Detector: UV 220/254 nm; Retention Time 1: 7.51 min; Retention Time 2: 11.52 min. The faster-eluting isomer at 7.51 min was obtained to afford Compound 57 ((3R,8R,9aS)-8-(2,3-dichloro-6-hydroxyphenyl)-3-(hydroxymethyl)-octahydropyrido[1,2-a]pyrazin-4-one) as an off-white solid (1.9 mg, 16%): LCMS (ESI) calc'd for C₁₅H₁₈Cl₂N₂O₃ [M+H]⁺: 345, 347 (3:2) found 345, 347 (3:2); ¹H NMR (400 MHz, CD₃OD) δ 7.19 (d, J=8.7 Hz, 1H), 6.71 (d, J=8.8 Hz, 1H), 4.82-4.73 (m, 1H), 3.96-3.84 (m, 2H), 3.72-3.54 (m, 2H), 3.45 (dd, J=5.7, 3.9 Hz, 1H), 3.36-3.34 (m, 1H), 2.82-2.62 (m, 2H), 2.53-2.24 (m, 2H), 1.66 (t, J=11.9 Hz, 2H). The slower-eluting isomer at 11.52 min was obtained to afford Compound 36 ((3R,8S,9aR)-8-(2,3-dichloro-6-hydroxyphenyl)-3-(hydroxymethyl)-octahydropyrido[1,2-a]pyrazin-4-one) as an off-white solid (2.6 mg, 21%): LCMS (ESI) calc'd for C₁₅H₁₈Cl₂N₂O₃ [M+H]⁺: 345, 347 (3:2) found 345, 347 (3:2); ¹H NMR (400 MHz, CD₃OD) δ 7.19 (d, J=8.7 Hz, 1H), 6.71 (d, J=8.8 Hz, 1H), 4.79-4.71 (m, 1H), 3.96 (dd, J=11.0, 6.6 Hz, 1H), 3.83 (dd, J=11.0, 3.8 Hz, 1H), 3.77-3.71 (m, 1H), 3.55-3.47 (m, 1H), 3.45 (dd, J=6.6, 3.7 Hz, 1H), 3.18 (dd, J=13.4, 5.0 Hz, 1H), 3.01 (dd, J=13.5, 5.2 Hz, 1H), 2.71 (td, J=13.1, 2.9 Hz, 1H), 2.62-2.58 (m, 1H), 2.53-2.39 (m, 1H), 1.60 (dt, J=13.2, 3.4 Hz, 2H).

### Example 26. Compound 46 ((3S,8R,9aS)-8-(2,3-dichloro-6-hydroxyphenyl)-2-(2-hydroxyacetyl)-3-methyloctahydro-4H-pyrido[1,2-a]pyrazin-4-one)

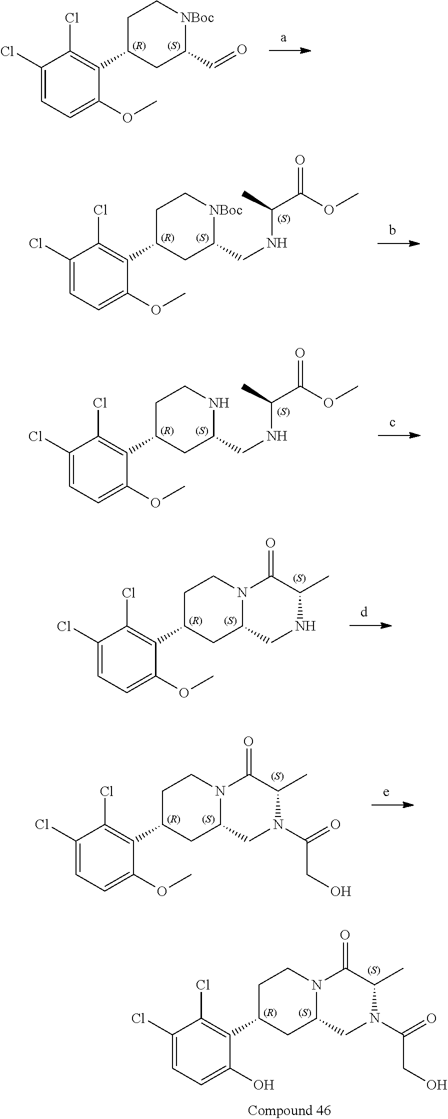

Step a:

To a stirred solution of tert-butyl (2S,4R)-4-(2,3-dichloro-6-methoxyphenyl)-2-formylpiperidine-1-carboxylate (Intermediate 10, Example 8) (200 mg, 0.51 mmol) and methyl L-alaninate (63.0 mg, 0.620 mmol) in DCM (2 mL) were added TEA (100 mg, 1.03 mmol) and NaBH(AcO)₃ (330 mg, 1.54 mmol) at room temperature. The resulting mixture was stirred at room temperature for 3 h. The reaction was quenched with saturated aq. NH₄Cl (20 mL) followed by extraction with EA (3×20 mL). The combined organic phases were washed with brine (2×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford tert-butyl (2S,4R)-4-(2,3-dichloro-6-methoxyphenyl)-2-([[(2S)-1-methoxy-1-oxopropan-2-yl]amino]methyl)piperidine-1-carboxylate as a yellow oil (180 mg, 73%): LCMS (ESI) calc'd for C₂₂H₃₂Cl₂N₂O₅ [M+H]⁺: 475, 477 (3:2) found 475, 477 (3:2); ¹H NMR (400 MHz, CDCl₃) δ 7.33 (d, J=8.9 Hz, 1H), 6.77 (d, J=8.9 Hz, 1H), 4.28-4.15 (m, 2H), 3.87 (s, 3H), 3.84 (s, 3H), 3.72-3.59 (m, 1H), 3.55-3.33 (m, 2H), 3.09 (d, J=12.4 Hz, 1H), 2.40-2.25 (m, 1H), 2.01-1.83 (m, 2H), 1.64 (d, J=7.1 Hz, 2H), 1.53 (s, 9H), 1.48 (d, J=3.8 Hz, 3H).

Step b:

To a stirred solution of tert-butyl (2S,4R)-4-(2,3-dichloro-6-methoxyphenyl)-2-([[(2S)-1-methoxy-1-oxopropan-2-yl]amino]methyl)piperidine-1-carboxylate (180 mg, 0.39 mmol) in DCM (2 mL) was added TFA (1 mL) at room temperature. The reaction solution was stirred at room temperature for 1 h. The reaction was quenched with saturated aq. NaHCO₃ (5 mL), diluted with water (10 mL) and extracted with EA (3×10 mL) respectively. The combined organic layers were washed with brine (3×10 mL) and dried over anhydrous Na₂SO₄. After filtration, the filtrate was concentrated under reduced pressure to afford methyl (2S)-2-([[(2S,4R)-4-(2,3-dichloro-6-methoxyphenyl)piperidin-2-yl]methyl]amino)propanoate as a yellow oil (0.20 g, crude), which was used directly in the next step without purification: LCMS (ESI) calc'd for C₁₇H₂₄Cl₂N₂O₃ [M+H]⁺: 375, 377 (3:2) found 375, 377 (3:2).

Step c:

To a stirred solution of methyl (2S)-2-([[(2S,4R)-4-(2,3-dichloro-6-methoxyphenyl)piperidin-2-yl]methyl]amino)propanoate (200 mg, 0.53 mmol) in EtOH (2 mL) was added TEA (160 mg, 1.60 mmol) at room temperature. The reaction solution was stirred at 80° C. for 1 h. The resulting mixture was concentrated under reduced pressure. The residue was purified by reverse phase chromatography, eluting with 55% ACN in water (plus 10 mM NH₄HCO₃) to afford (3S,8R,9aS)-8-(2,3-dichloro-6-methoxyphenyl)-3-methyl-octahydropyrido[1,2-a]pyrazin-4-one as a yellow oil (50.0 mg, 38% over two steps): LCMS (ESI) calc'd for C₁₆H₂OCl₂N₂O₂ [M+H]⁺: 343, 345 (3:2) found 343, 345 (3:2); ¹H NMR (400 MHz, CDCl₃) δ 7.31 (d, J=8.9 Hz, 1H), 6.75 (d, J=9.0 Hz, 1H), 4.90-4.79 (m, 1H), 3.82 (s, 3H), 3.77-3.64 (m, 1H), 3.58 (q, J=7.0 Hz, 1H), 3.45-3.37 (m, 1H), 3.21 (dd, J=13.3, 5.1 Hz, 1H), 3.00-2.89 (m, 1H), 2.61 (td, J=12.8, 2.8 Hz, 1H), 2.48-2.27 (m, 2H), 1.72-1.54 (m, 2H), 1.47 (d, J=7.0 Hz, 3H).

Step d:

To a stirred solution of glycolic acid (16.0 mg, 0.22 mmol), HOBT (29.0 mg, 0.22 mmol), and EDCI (42.0 mg, 0.219 mmol) in DMF (1 mL) were added (3S,8R,9aS)-8-(2,3-dichloro-6-methoxyphenyl)-3-methyl-octahydropyrido[1,2-a]pyrazin-4-one (50 mg, 0.15 mmol) and TEA (44.0 mg, 0.44 mmol) at room temperature. The resulting mixture was stirred at room temperature for 2 h, diluted with water (10 mL) and extracted with EA (3×20 mL). The combined organic layers were washed with brine (5×20 mL) and dried over anhydrous Na₂SO₄. After filtration, the filtrate was concentrated under reduced pressure to afford (3S,8R,9aS)-8-(2,3-dichloro-6-methoxyphenyl)-2-(2-hydroxyacetyl)-3-methyl-hexahydro-1H-pyrido[1,2-a]pyrazin-4-one as a yellow oil (90 mg, crude), which was used directly in the next step without purification: LCMS (ESI) calc'd for C₁₈H₂₂Cl₂N₂O₄ [M+H]⁺: 401, 403 (3:2) found 401, 403 (3:2).

Step e:

To a stirred solution of (3S,8R,9aS)-8-(2,3-dichloro-6-methoxyphenyl)-2-(2-hydroxyacetyl)-3-methyl-hexahydro-1H-pyrido[1,2-a]pyrazin-4-one (90.0 mg, 0.22 mmol) in DCM (1 mL) was added BBr₃ (0.25 mL) at room temperature. The resulting mixture was stirred at room temperature for 1 h, quenched with MeOH (2 mL) at room temperature and concentrated under reduced pressure. The residue was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 20% to 49% in 8 min; Detector: UV 254/220 nm; Retention time: 6.58 min. The fractions containing the desired product were collected and concentrated under reduced pressure to afford Compound 46 ((3S,8R,9aS)-8-(2,3-dichloro-6-hydroxyphenyl)-2-(2-hydroxyacetyl)-3-methyl-hexahydro-1H-pyrido[1,2-a]pyrazin-4-one) as an off-white solid (8.7 mg, 15% over two steps): LCMS (ESI) calc'd for C₁₇H₂₀Cl₂N₂O₄ [M+H]⁺: 387, 389 (3:2) found 387, 389 (3:2); ¹H NMR (400 MHz, CD₃OD) δ 7.21 (d, J=8.8 Hz, 1H), 6.73 (d, J=8.8 Hz, 1H), 5.00-4.94 (m, 1H), 4.78-4.69 (m, 1H), 4.65-4.40 (m, 1H), 4.40-4.14 (m, 2H), 3.95 (dd, J=14.5, 4.5 Hz, 1H), 3.75-3.52 (m, 2H), 3.09-2.64 (m, 1H), 2.57-2.38 (m, 1H), 2.38-2.20 (m, 1H), 1.83-1.52 (m, 3H), 1.47 (d, J=7.1 Hz, 2H).

### Example 27. Compound 12 ((3R,8R,9aS)-8-(2,3-dichloro-6-hydroxyphenyl)-2-(2-hydroxyacetyl)-3-methyloctahydro-4H-pyrido[1,2-a]pyrazin-4-one)

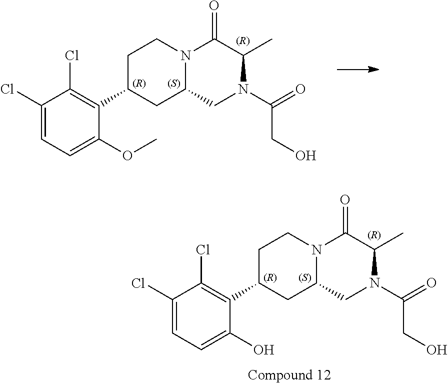

(3R,8R,9aS)-8-(2,3-dichloro-6-methoxyphenyl)-2-(2-hydroxyacetyl)-3-methyl-hexahydro-1H-pyrido[1,2-a]pyrazin-4-one was prepared by the same method as the preceding Example using methyl D-alaninate.

To a stirred solution of (3R,8R,9aS)-8-(2,3-dichloro-6-methoxyphenyl)-2-(2-hydroxyacetyl)-3-methyl-hexahydro-1H-pyrido[1,2-a]pyrazin-4-one (70.0 mg, 0.17 mmol) in DCM (1 mL) was added BBr₃ (0.25 mL) at room temperature. The resulting mixture was stirred at room temperature for 1 h, quenched with MeOH (2 mL) at room temperature and concentrated under reduced pressure. The residue was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 20% to 48% in 8 min; Detector: UV 254/220 nm. Retention time: 7.13 min. The fractions containing the desired product were collected and concentrated under reduced pressure to afford Compound 12 ((3R,8R,9aS)-8-(2,3-dichloro-6-hydroxyphenyl)-2-(2-hydroxyacetyl)-3-methyl-hexahydro-1H-pyrido[1,2-a]pyrazin-4-one) as an off-white solid (6.6 mg, 15% overall two steps): LCMS (ESI) calc'd for C₁₇H₂₀Cl₂N₂O₄ [M+H]⁺: 387, 389 (3:2) found 387, 389 (3:2); ¹H NMR (400 MHz, CD₃OD) δ 7.20 (d, J=8.8 Hz, 1H), 6.69 (d, J=8.8 Hz, 1H), 5.02-4.92 (m, 1H), 4.74-4.54 (m, 1H), 4.50-4.30 (m, 1H), 4.30-4.18 (m, 2H), 3.94-3.66 (m, 2H), 3.62 (d, J=11.7 Hz, 1H), 2.86-2.73 (m, 1H), 2.60-2.33 (m, 2H), 1.74-1.51 (m, 3H), 1.45 (d, J=7.1 Hz, 2H).

### Example 28. Compound 83 ((8R,9aS)-8-(2,3-dichloro-6-hydroxyphenyl)-2-(2-hydroxyacetyl)-1-methyl-hexahydro-1H-pyrido[1,2-a]pyrazin-4-one isomer 1) and Compound 84 ((8R,9aS)-8-(2,3-dichloro-6-hydroxyphenyl)-2-(2-hydroxyacetyl)-1-methyl-hexahydro-1H-pyrido[1,2-a]pyrazin-4-one isomer 2)

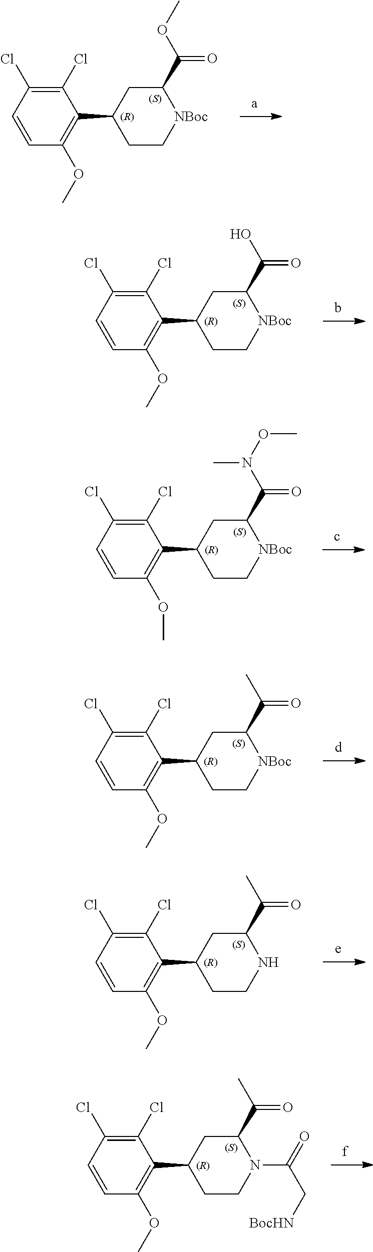

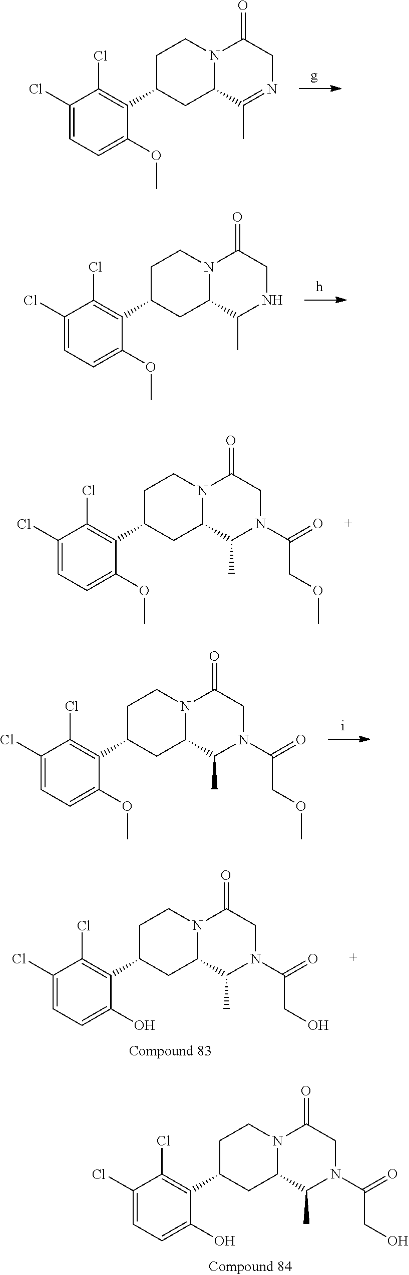

Step a:

To a solution of 1-tert-butyl 2-methyl (2S,4R)-4-(2,3-dichloro-6-methoxyphenyl)piperidine-1,2-dicarboxylate (2.00 g, 4.78 mmol) in MeOH (20 mL) and H₂O (1.00 mL) was added LiOH.H₂O (600 mg, 14.3 mmol) at room temperature. The reaction was stirred at room temperature for 12 h. Then the reaction was acidified with saturated aq. citric acid to pH 3 and the mixture was extracted with EA (3×30 mL). The combined organic phases were washed with brine (3×50 mL) and dried over anhydrous Na₂SO₄. After filtration, the filtrate was concentrated under reduced pressure to afford (2S,4R)-1-(tert-butoxycarbonyl)-4-(2,3-dichloro-6-methoxyphenyl)piperidine-2-carboxylic acid as a light yellow solid (1.90 g, 89%): LCMS (ESI) calc'd for C₁₈H₂₃Cl₂NO₅ [M+H]⁺: 404, 406 (3:2), found 404, 406 (3:2); ¹H NMR (400 MHz, CD₃OD) δ 7.38 (d, J=9.0 Hz, 1H), 6.97 (d, J=8.9 Hz, 1H), 4.25-4.20 (m, 1H), 3.86 (s, 3H), 3.82-3.47 (m, 3H), 2.60-2.56 (m, 1H), 2.18-1.99 (m, 1H), 1.99-1.81 (m, 2H), 1.40 (s, 9H).

Step b:

A solution of (2S,4R)-1-(tert-butoxycarbonyl)-4-(2,3-dichloro-6-methoxyphenyl)piperidine-2-carboxylic acid (1.00 g, 2.47 mmol), EDCI (710 mg, 3.71 mmol) and HOBT (500 mg, 3.71 mmol) in DMF (10 mL) was stirred for 30 min at room temperature. To the above solution were added TEA (1.03 mL, 10.19 mmol) and N,O-dimethylhydroxylamine hydrochloride (480 mg, 4.95 mmol) at room temperature. The reaction was stirred at room temperature for 3 h. The resulting mixture was diluted with water (40 mL) followed by extraction with EA (3×30 mL). The combined organic layers were washed with brine (3×30 mL) and dried over anhydrous Na₂SO₄. After filtration, the filtrate was concentrated under reduced pressure. The residue was purified by silica gel column chromatography, eluting with EA/PE (1/1) to afford tert-butyl (2S,4R)-4-(2,3-dichloro-6-methoxyphenyl)-2-[methoxy(methyl)carbamoyl]piperidine-1-carboxylate as a light yellow oil (400 mg, 36%): LCMS (ESI) calc'd for C₂₀H₂₈Cl₂N₂O₅ [M+H]⁺: 447, 449 (3:2), found 447, 449 (3:2); ¹H NMR (400 MHz, CDCl₃) δ 7.30 (d, J=8.8 Hz, 1H), 6.75 (d, J=8.9 Hz, 1H), 4.86-4.52 (m, 1H), 4.17-3.88 (m, 1H), 3.82 (s, 3H), 3.79 (s, 3H), 3.66-3.62 (m, 2H), 3.21 (s, 3H), 2.66-2.36 (m, 1H), 2.14-1.77 (m, 3H), 1.49 (s, 9H).

Step c:

To a solution of tert-butyl (2S,4R)-4-(2,3-dichloro-6-methoxyphenyl)-2-[methoxy(methyl)carbamoyl]piperidine-1-carboxylate (400 mg, 0.89 mmol) in THE (4 mL) was added MeMgBr (3.58 mL, 3.58 mmol, 1 M in THF) at 0° C. under nitrogen atmosphere. The reaction was stirred at room temperature for 2 h under nitrogen atmosphere. The reaction was quenched with saturated aq. NH₄Cl (20 mL) and extracted with EA (2×20 mL). The combined organic phases were washed with brine (2×20 mL) and dried over anhydrous Na₂SO₄. After filtration, the filtrate was concentrated under reduced pressure to afford tert-butyl (2S,4R)-2-acetyl-4-(2,3-dichloro-6-methoxyphenyl)piperidine-1-carboxylate as a light yellow oil (0.36 g, crude), which was used directly in the next step without further purification: LCMS (ESI) calc'd for C₁₉H₂₅Cl₂NO₄ [M+H]⁺: 402, 404 (3:2) found 402, 404 (3:2).

Step d:

To a stirred solution of tert-butyl (2S,4R)-2-acetyl-4-(2,3-dichloro-6-methoxyphenyl)piperidine-1-carboxylate (360 mg, 0.90 mmol) in DCM (4 mL) was added TFA (1 mL) dropwise at room temperature. The reaction was stirred at room temperature for 1 h. The resulting solution was concentrated under reduced pressure to afford 1-[(2S,4R)-4-(2,3-dichloro-6-methoxyphenyl)piperidin-2-yl]ethanone as a light yellow oil (280 mg, crude), which was used directly in the next step without further purification: LCMS (ESI) calc'd for C₁₄H₁₇Cl₂NO₂ [M+H]⁺: 302, 304 (3:2) found 302, 304 (3:2); ¹H NMR (400 MHz, CD₃OD) δ 7.45 (d, J=9.0 Hz, 1H), 7.02 (d, J=9.0 Hz, 1H), 4.27 (dd, J=12.8, 3.4 Hz, 1H), 3.90-3.88 (m, 1H), 3.87 (s, 3H), 3.57-3.50 (m, 1H), 3.16 (td, J=13.1, 3.3 Hz, 1H), 2.65-2.43 (m, 2H), 2.41-2.32 (m, 1H), 2.29 (s, 3H), 1.82 (d, J=14.3 Hz, 1H).

Step e:

To a solution of [(tert-butoxycarbonyl)amino]acetic acid (240 mg, 1.39 mmol) and HATU (530 mg, 1.39 mmol,) in DMF (3 mL) were added TEA (0.39 mL, 3.82 mmol) and 1-[(2S,4R)-4-(2,3-dichloro-6-methoxyphenyl)piperidin-2-yl]ethanone (280 mg, 0.93 mmol) at room temperature. The reaction was stirred at room temperature for 2 h. The resulting mixture was diluted with water (20 mL) and extracted with EA (3×20 mL). The combined organic layers were washed with brine (5×20 mL) and dried over anhydrous Na₂SO₄. After filtration, the filtrate was concentrated under reduced pressure to afford tert-butyl N-[2-[(2S,4R)-2-acetyl-4-(2,3-dichloro-6-methoxyphenyl)piperidin-1-yl]-2-oxoethyl]carbamate as a yellow oil (500 mg, crude), which was used directly in the next step without further purification: LCMS (ESI) calc'd for C₂₁H₂₈Cl₂N₂O₅ [M+H]⁺: 459, 461 (3:2) found 459, 461 (3:2); ¹H NMR (400 MHz, CDCl₃) δ 7.33 (d, J=8.9 Hz, 1H), 6.77 (d, J=8.9 Hz, 1H), 4.44 (s, 1H), 4.10-4.01 (m, 2H), 3.83 (s, 3H), 3.75-3.66 (m, 2H), 3.60-3.47 (m, 1H), 2.52-2.32 (m, 1H), 2.23 (s, 3H), 2.18-2.08 (m, 1H), 2.01-1.89 (m, 2H), 1.47 (s, 9H).

Step f:

To a solution of tert-butyl N-[2-[(2S,4R)-2-acetyl-4-(2,3-dichloro-6-methoxyphenyl)piperidin-1-yl]-2-oxoethyl]carbamate (500 mg, 1.09 mmol) in DCM (4 mL) was added TFA (1 mL) at room temperature. The reaction was stirred at room temperature for 30 min. The reaction mixture was basified with aq. NaHCO₃ to pH 7. Then the resulting mixture was extracted with DCM (2×20 mL). The combined organic phases were washed with brine (2×20 mL) and dried over anhydrous Na₂SO₄. After filtration, the filtrate was concentrated under reduced pressure to afford (8R,9aS)-8-(2,3-dichloro-6-methoxyphenyl)-1-methyl-3H,6H,7H,8H,9H,9aH-pyrido[1,2-a]pyrazin-4-one as a light yellow oil (360 mg, crude): LCMS (ESI) calc'd for C₁₆H₁₈Cl₂N₂O₂ [M+H]⁺: 341, 343 (3:2), found 341, 343 (3:2); ¹H NMR (400 MHz, CD₃OD) δ 7.41 (d, J=9.0 Hz, 1H), 6.98 (d, J=9.0 Hz, 1H), 4.81-4.72 (m, 1H), 4.27-4.18 (m, 2H), 3.84 (s, 3H), 2.83 (s, 2H), 2.74 (td, J=13.1, 3.0 Hz, 1H), 2.39-2.26 (m, 2H), 2.16-2.08 (m, 1H), 2.06 (t, J=1.7 Hz, 3H), 1.64 (d, J=13.3 Hz, 1H).

Step g:

To a solution of (8R,9aS)-8-(2,3-dichloro-6-methoxyphenyl)-1-methyl-3H,6H,7H,8H,9H,9aH-pyrido[1,2-a]pyrazin-4-one (360 mg, 1.06 mmol) in MeOH (2 mL) was added PtO₂ (24.0 mg, 0.11 mmol) at room temperature. The mixture was stirred at room temperature for 12 h under hydrogen atmosphere (1.5 atm). The reaction mixture was filtered through a Celite pad and the filtrate was concentrated under reduced pressure. The residue was purified with reverse phase chromatography, eluting with 32% MeCN in water (plus 0.05% TFA) to afford (8R,9aS)-8-(2,3-dichloro-6-methoxyphenyl)-1-methyl-octahydropyrido[1,2-a]pyrazin-4-one; trifluoroacetic acid as a light yellow oil (300 mg, 73%): LCMS (ESI) calc'd for C₁₆H₂OCl₂N₂O₂ [M+H]⁺: 343, 345 (3:2), found 343, 345 (3:2); ¹H NMR (400 MHz, CD₃OD) δ 7.41 (d, J=8.9 Hz, 1H), 6.98 (d, J=8.8 Hz, 1H), 4.65-4.51 (m, 1H), 4.44-4.33 (m, 1H), 3.86-3.81 (m, 4H), 3.68-3.55 (m, 3H), 3.26-3.11 (m, 1H), 2.24-2.11 (m, 1H), 1.99-1.90 (m, 1H), 1.78-1.69 (m, 1H), 1.53-1.43 (m, 1H), 1.36 (d, J=6.6 Hz, 3H).

Step h:

To a solution of methoxyacetic acid (120 mg, 1.33 mmol) in DMF (3 mL) and HATU (580 mg, 1.53 mmol) were added (8R,9aS)-8-(2,3-dichloro-6-methoxyphenyl)-1-methyl-octahydropyrido[1,2-a]pyrazin-4-one (350 mg, 1.02 mmol) and TEA (310 mg, 3.06 mmol) at room temperature. The reaction was stirred at room temperature for 2 h, poured into water (40 mL) and extracted with EA (2×30 mL). The combined organic phases were washed with brine (4×30 mL) and dried over anhydrous Na₂SO₄. After filtration, the filtrate was concentrated under reduced pressure. The residue was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 25% B to 55% B in 7 min; Detector: UV 220 nm; Retention Time 1: 6.35 min; Retention Time 2: 6.55 min; The faster-eluting enantiomer at 6.35 min was obtained (8R,9aS)-8-(2,3-dichloro-6-methoxyphenyl)-2-(2-methoxyacetyl)-1-methyl-hexahydro-1H-pyrido[1,2-a]pyrazin-4-one isomer 1 as a light yellow foam (80.0 mg, 25%): LCMS (ESI) calc'd for C₁₉H₂₄Cl₂N₂O₄ [M+H]⁺: 415, 417 (3:2), found 415, 417 (3:2); ¹H NMR (400 MHz, CDCl₃) δ 7.27 (d, J=9.1 Hz, 1H), 6.71 (d, J=9.2 Hz, 1H), 5.65-5.18 (m, 1H), 5.03-4.60 (m, 2H), 4.56-3.89 (m, 3H), 3.86-3.61 (m, 4H), 3.59-3.19 (m, 4H), 2.67 (t, J=12.8 Hz, 1H), 2.44-2.17 (m, 2H), 1.62 (dd, J=38.6, 13.0 Hz, 2H), 1.50-1.23 (m, 3H). The slower-eluting enantiomer at 6.55 min was obtained (8R,9aS)-8-(2,3-dichloro-6-methoxyphenyl)-2-(2-methoxyacetyl)-1-methyl-hexahydro-1H-pyrido[1,2-a]pyrazin-4-one isomer 2 as a light yellow foam (80 mg, 25%): LCMS (ESI) calc'd for C₁₉H₂₄Cl₂N₂O₄ [M+H]⁺: 415, 417 (3:2) found 415, 417 (3:2); ¹H NMR (400 MHz, CDCl₃) δ 7.29 (d, J=8.9 Hz, 1H), 6.74 (d, J=8.9 Hz, 1H), 5.67-5.19 (m, 1H), 4.97-4.60 (m, 2H), 4.41-3.93 (m, 3H), 3.80 (s, 3H), 3.74-3.47 (m, 2H), 3.40 (s, 3H), 2.76 (t, J=12.7 Hz, 1H), 2.45-2.07 (m, 2H), 1.63 (dd, J=54.4, 13.0 Hz, 2H), 1.39-1.16 (m, 3H).

Step i:

To a solution of (8R,9aS)-8-(2,3-dichloro-6-methoxyphenyl)-2-(2-methoxyacetyl)-1-methyl-hexahydro-1H-pyrido[1,2-a]pyrazin-4-one isomer 1 or (8R,9aS)-8-(2,3-dichloro-6-methoxyphenyl)-2-(2-methoxyacetyl)-1-methyl-hexahydro-1H-pyrido[1,2-a]pyrazin-4-one isomer 2 (80.0 mg, 0.19 mmol) in DCM (2 mL) was added BBr₃ (0.15 mL, 1.59 mmol) at room temperature. The reaction was stirred at room temperature for 2 h. The reaction was quenched with MeOH (5 mL). The mixture was concentrated under reduced pressure. The residue was purified by Prep-HPLC with the following conditions: Column: XBridge Shield RP18 OBD Column, 30×150 mm, 5 μm; Mobile Phase A: water (plus 0.05% TFA), Mobile Phase B: ACN; Flow rate: 60 mL/min; Gradient: 23% B to 50% B in 7 min; Detector: UV 220 nm; Retention time: 4.15 min for both (8R,9aS)-8-(2,3-dichloro-6-hydroxyphenyl)-2-(2-hydroxyacetyl)-1-methyl-hexahydro-1H-pyrido[1,2-a]pyrazin-4-one isomer 1 and (8R,9aS)-8-(2,3-dichloro-6-hydroxyphenyl)-2-(2-hydroxyacetyl)-1-methyl-hexahydro-1H-pyrido[1,2-a]pyrazin-4-one isomer 2. The fractions containing the desired product were collected and concentrated under reduced pressure to afford Compound 83 ((8R,9aS)-8-(2,3-dichloro-6-hydroxyphenyl)-2-(2-hydroxyacetyl)-1-methyl-hexahydro-1H-pyrido[1,2-a]pyrazin-4-one isomer 1) as an off-white solid (33.4 mg, 45%): LCMS (ESI) calc'd for C₁₇H₂OCl₂N₂O₄ [M+H]⁺: 387, 389 (3:2) found 387, 389 (3:2); ¹H NMR (400 MHz, CD₃OD) δ 7.19 (d, J=8.8 Hz, 1H), 6.69 (d, J=8.7 Hz, 1H), 4.79-4.65 (m, 2H), 4.37-4.17 (m, 2H), 4.09-4.05 (m, 1H), 3.95-3.66 (m, 2H), 3.50 (d, J=11.2 Hz, 1H), 2.80 (td, J=13.0, 2.9 Hz, 1H), 2.54-2.34 (m, 2H), 1.71 (dd, J=35.1, 12.6 Hz, 1H), 1.57 (d, J=13.3 Hz, 1H), 1.47-1.32 (m, 3H).

The fractions containing the desired product were collected and concentrated under reduced pressure to afford Compound 84 ((8R,9aS)-8-(2,3-dichloro-6-hydroxyphenyl)-2-(2-hydroxyacetyl)-1-methyl-hexahydro-1H-pyrido[1,2-a]pyrazin-4-one isomer 2) as an off-white solid (41.4 mg, 56%): LCMS (ESI) calc'd for C₁₇H₂OCl₂N₂O₄ [M+H]⁺: 387, 389 (3:2) found 387, 389 (3:2); ¹H NMR (400 MHz, CD₃OD) δ 7.21 (d, J=8.8 Hz, 1H), 6.73 (d, J=8.8 Hz, 1H), 4.85-4.59 (m, 2H), 4.41-4.10 (m, 3H), 3.90-3.57 (m, 3H), 2.90-2.70 (m, 1H), 2.56-2.35 (m, 2H), 1.68 (dd, J=29.2, 13.1 Hz, 2H), 1.39-1.19 (m, 3H).

### Example 29. Compound 85 ((3S,7R,8aS)-7-(2,3-dichloro-6-hydroxyphenyl)-3-methyl-hexahydro-1H-pyrrolo[1,2-a]pyrazin-4-one)

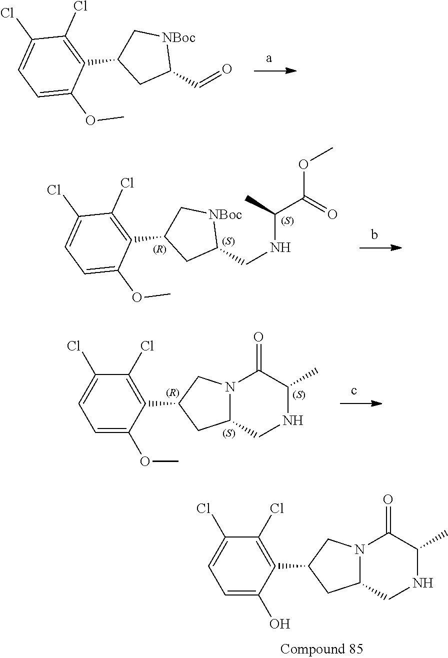

Step a:

To a stirred solution of tert-butyl (2S,4R)-4-(2,3-dichloro-6-methoxyphenyl)-2-formylpyrrolidine-1-carboxylate (Example 7, step c) (500 mg, 1.34 mmol) and D-alanyl ester hydrochloride (370 mg, 2.65 mmol) in DCM (5 mL) were added TEA (340 mg, 3.36 mmol) and NaBH(AcO)₃ (570 mg, 2.69 mmol) at room temperature. The reaction was stirred at room temperature for 2 h, quenched with water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by reverse phase chromatography, eluting with 45% ACN in water (plus 0.05% TFA) to afford tert-butyl (2S,4R)-4-(2,3-dichloro-6-methoxyphenyl)-2-([[(2S)-1-methoxy-1-oxopropan-2-yl]amino]methyl)pyrrolidine-1-carboxylate as a yellow oil (300 mg, 48%): LCMS (ESI) calc'd for C₂₁H₃₀Cl₂N₂O₅ [M+H]⁺ 461, 463 (3:2) found 461, 463 (3:2); ¹H NMR (400 MHz, CD₃OD) δ 7.46 (d, J=9.0 Hz, 1H), 7.03 (d, J=9.0 Hz, 1H), 4.31-4.10 (m, 3H), 3.92 (s, 3H), 3.89 (s, 3H), 3.88-3.71 (m, 2H), 3.45-3.37 (m, 1H), 3.24-2.90 (m, 1H), 2.53-2.33 (m, 2H), 1.63 (d, J=7.2 Hz, 3H), 1.52 (s, 9H).

Step b:

To a stirred solution of tert-butyl (2S,4R)-4-(2,3-dichloro-6-methoxyphenyl)-2-([[(2S)-1-methoxy-1-oxopropan-2-yl]amino]methyl)pyrrolidine-1-carboxylate (300 mg, 0.65 mmol) in DCM (5 mL) was added TFA (1 mL) at room temperature. The reaction was stirred at room temperature for 1 h. The resulting reaction was concentrated under reduced pressure. The residue was dissolved in EtOH (5 mL) and TEA (200 mg, 1.95 mmol) was added. The reaction was stirred at 80° C. for 1 h. After being allowed to cool to room temperature, the resulting mixture was diluted with water (30 mL). The solution was extracted with EA (3×50 mL). The combined organic layers were washed with brine (2×50 mL) and dried over anhydrous Na₂SO₄. After filtration, the filtrate was concentrated under reduced pressure to afford (3S,7R,8aS)-7-(2,3-dichloro-6-methoxyphenyl)-3-methyl-hexahydro-1H-pyrrolo[1,2-a]pyrazin-4-one as a yellow oil (220 mg, 95%): LCMS (ESI) calc'd for C₁₅H₁₈Cl₂N₂O₂ [M+H]⁺ 329, 331 (3:2) found 329, 331 (3:2); ¹H NMR (400 MHz, CD₃OD) δ 7.43 (d, J=9.0 Hz, 1H), 7.00 (d, J=9.0 Hz, 1H), 4.35-4.27 (m, 2H), 3.85 (s, 3H), 3.64-3.48 (m, 4H), 3.26-3.12 (m, 2H), 2.90-2.79 (m, 1H), 1.48-1.44 (m, 3H).

Step c:

To a stirred solution of (3S,7R,8aS)-7-(2,3-dichloro-6-methoxyphenyl)-3-methyl-hexahydro-1H-pyrrolo[1,2-a]pyrazin-4-one (120 mg, 0.36 mmol) in DCM (3 mL) was added BBr₃ (550 mg, 2.20 mmol) at room temperature. The reaction was stirred at room temperature for 1 h. The reaction was quenched with MeOH (10 mL) and concentrated under reduced pressure. The residue was purified by reverse phase chromatography, eluting with 12% ACN in water (plus 0.05% TFA) to afford Compound 85 ((3S,7R,8aS)-7-(2,3-dichloro-6-hydroxyphenyl)-3-methyl-hexahydro-1H-pyrrolo[1,2-a]pyrazin-4-one) as a light yellow oil (80.0 mg, 51%): LCMS (ESI) calc'd for C₁₄H₁₆Cl₂N₂O₂ [M+H]⁺ 315, 317 (3:2) found 315, 317 (3:2); ¹H NMR (400 MHz, CD₃OD) δ 7.31 (d, J=8.8 Hz, 1H), 6.79 (d, J=8.8 Hz, 1H), 4.50-4.33 (m, 1H), 4.26-4.04 (m, 2H), 3.84-3.58 (m, 2H), 3.27-3.07 (m, 2H), 2.54-2.18 (m, 2H), 1.70-1.59 (m, 3H).

### Example 30. Compound 60 ((3S,7R,8aS)-7-(2,3-dichloro-6-hydroxyphenyl)-2-(2-hydroxyacetyl)-3-methyl-hexahydropyrrolo[1,2-a]pyrazin-4-one)

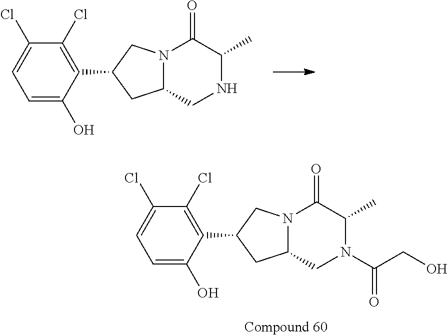 [SMILES: C[C@@H]1NC[C@@H]2C[C@H](c3c(O)ccc(Cl)c3Cl)CN2C1=O ; C[C@H]1C(=O)N2C[C@@H](c3c(O)ccc(Cl)c3Cl)C[C@H]2CN1C(=O)CO]

To a stirred solution of glycolic acid (12.0 mg, 0.159 mmol), EDCI (36.0 mg, 0.19 mmol) and HOBT (26.0 mg, 0.19 mmol) in DMF (2 mL) were added (3S,7R,8aS)-7-(2,3-dichloro-6-hydroxyphenyl)-3-methyl-hexahydro-1H-pyrrolo[1,2-a]pyrazin-4-one (Compound 85, Example 29) (50.0 mg, 0.16 mmol) and TEA (40.0 mg, 0.40 mmol) at room temperature. The reaction was stirred at room temperature for 16 h. The reaction was quenched with MeOH (0.5 mL) and was purified with Prep-HPLC with the following conditions: Column: Xselect CSH OBD Column 30×150 mm, 5 m; Mobile Phase A: water (plus 0.05% TFA), Mobile Phase B: ACN; Flow rate: 60 mL/min; Gradient: 20% B to 40% B in 7 min; Detector: UV 254/220 nm; Retentione time: 6.73 min. The fractions containing the desired product were collected and concentrated under reduced pressure to afford Compound 60 ((3S,7R,8aS)-7-(2,3-dichloro-6-hydroxyphenyl)-2-(2-hydroxyacetyl)-3-methyl-hexahydropyrrolo[1,2-a]pyrazin-4-one) as an off-white solid (14.5 mg, 24%): LCMS (ESI) calc'd for C₁₆H₁₈Cl₂N₂O₄ [M+H]⁺ 373, 375 (3:2) found 373, 375 (3:2); ¹H NMR (400 MHz, CD₃OD) δ 7.26 (d, J=8.7 Hz, 1H), 6.77 (d, J=8.8 Hz, 1H), 4.45-4.21 (m, 4H), 4.21-4.10 (m, 2H), 4.01-3.70 (m, 1H), 3.64-3.47 (m, 1H), 3.26-3.17 (m, 1H), 2.44-2.09 (m, 2H), 1.51 (d, J=7.0 Hz, 3H).

### Example 31. Compound 87 ((7R,8aS)-7-(2,3-dichloro-6-hydroxyphenyl)-2-(2-hydroxyacetyl)-1-methylhexahydropyrrolo[1,2-a]pyrazin-4(1H)-one isomer 1) and Compound 88 ((7R,8aS)-7-(2,3-dichloro-6-hydroxyphenyl)-2-(2-hydroxyacetyl)-1-methylhexahydropyrrolo[1,2-a]pyrazin-4(1H)-one isomer 2)

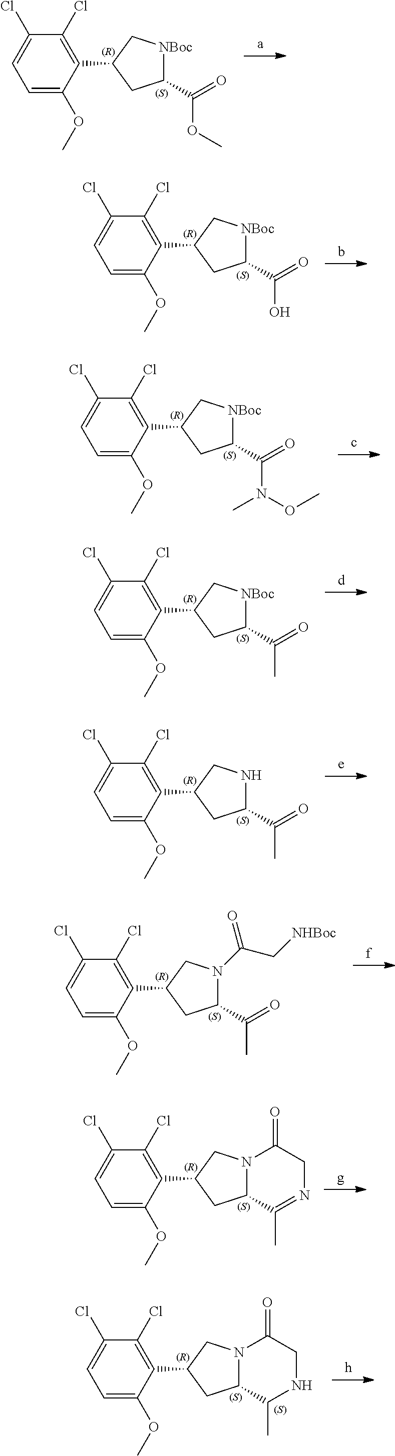

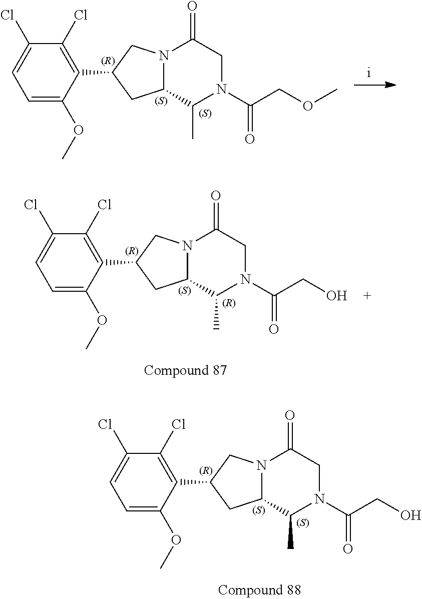

Step a:

To a stirred solution of 1-tert-butyl 2-methyl (2S,4R)-4-(2,3-dichloro-6-methoxyphenyl)pyrrolidine-1,2-dicarboxylate (Intermediate 7, Example 6) (2.00 g, 4.95 mmol) in MeOH (20 mL) was added LiOH H₂O (620 mg, 14.84 mmol) in H₂O (1 mL) at room temperature. Then the reaction was stirred at room temperature for 12 h and acidified with citric acid (30 mL) to pH 3 followed by extraction with EA (3×20 mL). The combined organic phases were washed with brine (2×20 mL) and dried over Na₂SO₄. After filtration, the filtrate was concentrated under reduced pressure to afford (2S,4R)-1-(tert-butoxycarbonyl)-4-(2,3-dichloro-6-methoxyphenyl)pyrrolidine-2-carboxylic acid as an off-white foam (1.60 g, 83%): LCMS (ESI) calc'd for C₁₇H₂₁Cl₂NO₅ [M+Na]⁺: 412, 414 (3:2) found 412, 414 (3:2); ¹H NMR (400 MHz, CDCl₃) δ 7.35 (d, J=8.9 Hz, 1H), 6.77 (d, J=9.0 Hz, 1H), 4.58-4.39 (m, 1H), 4.27-4.13 (m, 1H), 3.92-3.73 (m, 5H), 3.01-2.65 (m, 1H), 2.57-2.35 (m, 1H), 1.50 (s, 9H).

Step b:

To a solution of (2S,4R)-1-(tert-butoxycarbonyl)-4-(2,3-dichloro-6-methoxyphenyl)pyrrolidine-2-carboxylic acid (1.40 g, 3.59 mmol) in DMF (15 mL) were added EDCI (1.03 g, 5.38 mmol) and HOBT (720 mg, 5.38 mmol) at room temperature. 30 min later, N,O-dimethylhydroxylamine hydrochloride (700 mg, 7.18 mmol) and TEA (3 mL, 24.6 mmol) were added at 0° C. under nitrogen atmosphere. The reaction was stirred at room temperature for 2 h, diluted with water (80 mL) and extracted with EA (3×20 mL). The combined organic phases were washed with brine (2×50 mL) and dried over anhydrous Na₂SO₄. After filtration, the filtrate was concentrated under reduced pressure. The residue was purified by silica gel column chromatography, eluting with PE/EA (1/4) to afford tert-butyl (2S,4R)-4-(2,3-dichloro-6-methoxyphenyl)-2-[methoxy(methyl)carbamoyl]pyrrolidine-1-carboxylate as a light yellow oil (1.10 g, 71%): LCMS (ESI) calc'd for C₁₉H₂₆Cl₂N₂O₅ [M+H]⁺: 433, 435 (3:2) found 433, 435 (3:2); ¹H NMR (400 MHz, CDCl₃) δ 7.33 (d, J=8.9 Hz, 1H), 6.74 (d, J=8.9 Hz, 1H), 4.78 (s, 1H), 4.20-4.08 (m, 1H), 4.00-3.87 (m, 1H), 3.82 (d, J=3.4 Hz, 3H), 3.80-3.66 (m, 4H), 3.25 (s, 3H), 2.64-2.35 (m, 2H), 1.47 (d, J=11.1 Hz, 9H).

Step c:

To a solution of tert-butyl (2S,4R)-4-(2,3-dichloro-6-methoxyphenyl)-2-[methoxy(methyl)carbamoyl]pyrrolidine-1-carboxylate (1.10 g, 2.54 mmol) in THE (10 mL) was added MeMgBr (7.62 mL, 7.614 mmol, 1 M solution in THF) at 0° C. The reaction was stirred at room temperature for 1 h under nitrogen atmosphere and quenched with saturated aq. NH₄Cl (10 mL) followed by extraction with EA (3×50 mL). The combined organic phases were washed with brine (3×30 mL) and dried over anhydrous Na₂SO₄. After filtration, the filtrate was concentrated under reduced pressure to afford tert-butyl (2S,4R)-2-acetyl-4-(2,3-dichloro-6-methoxyphenyl)pyrrolidine-1-carboxylate as a yellow oil (630 mg, 89%): LCMS (ESI) calc'd for C₁₈H₂₃Cl₂NO₄ [M+Na]⁺: 410, 412 (3:2), found 410, 412 (3:2); ¹H NMR (400 MHz, CDCl₃) δ 7.36 (d, J=8.9 Hz, 1H), 6.78 (d, J=8.9 Hz, 1H), 4.43 (t, J=8.7 Hz, 1H), 4.27-4.17 (m, 1H), 3.93 (t, J=10.3 Hz, 1H), 3.87-3.68 (m, 4H), 2.60-2.45 (m, 1H), 2.40-2.28 (m, 1H), 2.22 (d, J=5.1 Hz, 3H), 1.48 (d, J=11.7 Hz, 9H).

Step d:

To a solution of tert-butyl (2S,4R)-2-acetyl-4-(2,3-dichloro-6-methoxyphenyl)pyrrolidine-1-carboxylate (630 mg) in DCM (4 mL) was added TFA (1 mL) at room temperature. The reaction was stirred at room temperature for 30 min and then concentrated under reduced pressure to afford 1-[(2S,4R)-4-(2,3-dichloro-6-methoxyphenyl)pyrrolidin-2-yl]ethanone as a light yellow oil (470 mg, crude), which was used directly in the next step without purification: LCMS (ESI) calc'd for C₁₃H₁₅Cl₂NO₂ [M+H]⁺: 288, 290 (3:2) found 288, 290 (3:2).

Step e:

To a solution of [(tert-butoxycarbonyl)amino]acetic acid (390 mg, 2.20 mmol) and HATU (840 mg, 2.20 mmol) in DMF (5 mL) were added 1-[(2S,4R)-4-(2,3-dichloro-6-methoxyphenyl)pyrrolidin-2-yl]ethanone (420 mg, 1.47 mmol) and TEA (0.6 mL, 6.05 mmol) at room temperature. The reaction was then stirred at room temperature for 1 h and poured into water (30 mL) followed by extraction with EA (2×50 mL). The combined organic phases were washed with brine (4×20 mL) and dried over anhydrous Na₂SO₄. After filtration, the filtrate was concentrated under reduced pressure. The residue was purified by silica gel column chromatography, eluting with EA/PE (3/2) to afford tert-butyl N-[2-[(2S,4R)-2-acetyl-4-(2,3-dichloro-6-methoxyphenyl)pyrrolidin-1-yl]-2-oxoethyl]carbamate as an off-white solid (580 mg, 80% over two steps): LCMS (ESI) calc'd for C₂₀H₂₆Cl₂N₂O₅ [M+H]⁺: 445, 447 (3:2) found 445, 447 (3:2); ¹H NMR (400 MHz, CDCl₃) δ 7.38 (d, J=8.9 Hz, 1H), 6.79 (d, J=9.0 Hz, 1H), 4.66 (t, J=8.8 Hz, 1H), 4.36-4.24 (m, 1H), 4.08-3.89 (m, 3H), 3.84 (s, 3H), 3.66 (t, J=8.8 Hz, 1H), 2.51-2.33 (m, 2H), 2.24 (s, 3H), 1.47 (d, J=5.2 Hz, 9H).

Step f:

To a solution of tert-butyl N-[2-[(2S,4R)-2-acetyl-4-(2,3-dichloro-6-methoxyphenyl)pyrrolidin-1-yl]-2-oxoethyl]carbamate (580 mg, 1.31 mmol) in DCM (4 mL) was added TFA (1 mL) at room temperature. The reaction was stirred at room temperature for 30 min. The reaction mixture was concentrated under reduced pressure to afford (7R,8aS)-7-(2,3-dichloro-6-methoxyphenyl)-1-methyl-3H,6H,7H,8H,8aH-pyrrolo[1,2-a]pyrazin-4-one as a light yellow oil (660 mg, crude), which was used directly in the next step without further purification: LCMS (ESI) calc'd for C₁₅H₁₆Cl₂N₂O₂ [M+H]⁺: 327, 329 (3:2) found 327, 329 (3:2).

Step g:

To a stirred solution of (7R,8aS)-7-(2,3-dichloro-6-methoxyphenyl)-1-methyl-3H,6H,7H,8H,8aH-pyrrolo[1,2-a]pyrazin-4-one (660 mg, 2.02 mmol) in MeOH (5 mL) was added NaBH₄ (150 mg, 4.05 mmol) at 0° C. under nitrogen atmosphere. The reaction was stirred at room temperature for 1 h. The resulting mixture was diluted with NH₄Cl (20 mL) and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography eluting with 50% ACN in water (plus 0.05% TFA) to afford (7R,8aS)-7-(2,3-dichloro-6-methoxyphenyl)-1-methyl-hexahydro-1H-pyrrolo[1,2-a]pyrazin-4-one as a light yellow oil (380 mg, 88% overall two steps): LCMS (ESI) calc'd for C₁₅H₁₈Cl₂N₂O₂ [M+H]⁺: 329, 331 (3:2) found 329, 331 (3:2); ¹H NMR (400 MHz, CD₃OD) δ 7.46 (dd, J=9.0, 1.7 Hz, 1H), 7.04 (dd, J=12.9, 8.9 Hz, 1H), 4.46-4.25 (m, 2H), 4.17-4.01 (m, 1H), 3.95-3.72 (m, 6H), 3.67-3.46 (m, 1H), 2.40-2.14 (m, 2H), 1.43 (dd, J=6.3, 4.9 Hz, 3H).

Step h:

A solution of methoxyacetic acid (150 mg, 1.64 mmol) and HATU (620 mg, 1.64 mmol) in DMF (8 mL) was stirred for 30 min at room temperature. Then to the mixture was added TEA (1 mL, 7.12 mmol) and (7R,8aS)-7-(2,3-dichloro-6-methoxyphenyl)-1-methyl-hexahydro-1H-pyrrolo[1,2-a]pyrazin-4-one (360 mg, 1.09 mmol) at room temperature. The reaction was stirred at room temperature for 1 h, diluted with water (30 mL) and extracted with EA (2×30 mL). The combined organic layers were washed with brine (5×30 mL) and dried over anhydrous Na₂SO₄. After filtration, the filtrate was concentrated under reduced pressure to afford (7R,8aS)-7-(2, 3-dichloro-6-methoxyphenyl)-2-(2-methoxyacetyl)-1-methyl-hexahydropyrrolo[1,2-a]pyrazin-4-one as a light yellow oil (350 mg, 80%) which was used directly in the next step without purification: LCMS (ESI) calc'd for C₁₈H₂₂Cl₂N₂O₄ [M+H]⁺: 401, 403 (3:2) found 401, 403 (3:2).

Step i:

To a stirred solution of (7R,8aS)-7-(2,3-dichloro-6-methoxyphenyl)-2-(2-methoxyacetyl)-1-methyl-hexahydropyrrolo[1,2-a]pyrazin-4-one (300 mg, 0.75 mmol) in DCM (10 mL) was added BBr₃ (5 mL) slowly at 0° C. The resulting mixture was stirred for 50 min at room temperature. The reaction was quenched with MeOH (5 mL) at 0° C. The resulting mixture was concentrated under reduced pressure. The residue was purified by Prep-HPLC with the following conditions: Column: SunFire Prep C18 OBD Column, 19×150 mm 5 μm 10 nm; Mobile Phase A: Water (plus 0.05% TFA), Mobile Phase B: ACN; Flow rate: 25 mL/min; Gradient: 23% B to 48% B in 11 min; UV: Detector 220 nm; Retention Time 1:10.05 min, Retention Time 2 10.6 min. The fractions containing the desired product at 10.05 min to afford Compound 87 ((7R,8aS)-7-(2,3-dichloro-6-hydroxyphenyl)-2-(2-hydroxyacetyl)-1-methyl-hexahydropyrrolo[1,2-a]pyrazin-4-one isomer 1) as an off-white solid (35 mg, 12.54%): LCMS (ESI) calc'd for C₁₆H₁₈Cl₂N₂O₄ [M+H]⁺: 373, 375 (3:2), found 373, 375 (3:2); ¹H NMR (400 MHz, CD₃OD) δ 7.27 (d, J=8.8 Hz, 1H), 6.76 (d, J=8.8 Hz, 1H), 4.43-4.15 (m, 3H), 4.07-3.79 (m, 6H), 2.98-2.81 (m, 1H), 2.16-2.07 (m, 1H), 1.33 (d, J=5.2 Hz, 3H); The fractions containing the desired product at 10.60 min were combined to afford Compound 88 ((7R,8aS)-7-(2,3-dichloro-6-hydroxyphenyl)-2-(2-hydroxyacetyl)-1-methyl-hexahydropyrrolo[1,2-a]pyrazin-4-one isomer 2) as an off-white solid (55 mg, 19.71%): LCMS (ESI) calc'd for C₁₆H₁₈Cl₂N₂O₄ [M+H]⁺: 373, 375 (3:2), found 373, 375 (3:2); ¹H NMR (400 MHz, CD₃OD) δ 7.27 (d, J=8.8 Hz, 1H), 6.76 (d, J=8.8 Hz, 1H), 4.43-4.15 (m, 4H), 4.14-4.02 (m, 1H), 4.01-3.85 (m, 2H), 3.83-3.66 (m, 2H), 2.58-2.53 (m, 1H), 2.40 (dt, J=11.8, 6.7 Hz, 1H), 1.35 (d, J=6.2 Hz, 3H).

### Example 32. Compound 18 ((8R,9aS)-8-(2,3-dichloro-6-hydroxyphenyl)-2-(2-hydroxyacetyl)-hexahydro-1H-pyrrolo[1,2-a][1,4]diazepin-5-one)

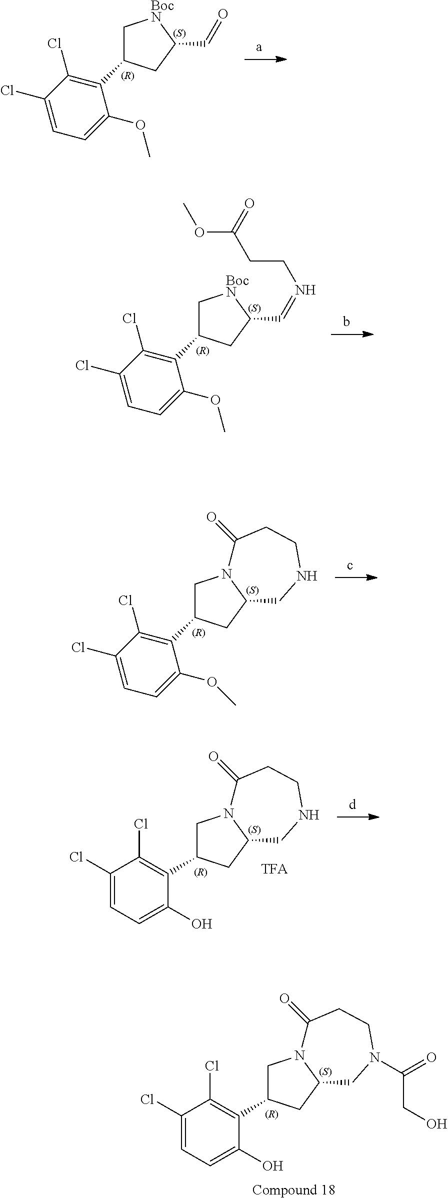

Step a:

To a stirred solution of tert-butyl (2S,4R)-4-(2,3-dichloro-6-methoxyphenyl)-2-formylpyrrolidine-1-carboxylate (Example 7, step c) (1.90 g, 5.08 mmol) and methyl 3-aminopropanoate hydrochloride (500 mg, 1.39 mmol) in DCM (20 mL) were added TEA (430 mg, 4.25 mmol) and NaBH(AcO)₃ (600 mg, 2.83 mmol) at room temperature. The reaction was stirred for 2 h, quenched with water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by reverse phase chromatography, eluting with 45% ACN in water (plus 0.05% TFA) to afford tert-butyl (2S,4R)-4-(2,3-dichloro-6-hydroxyphenyl)-2-[[(3-methoxy-3-oxopropyl)amino]methyl]pyrrolidine-1-carboxylate as a yellow oil (300 mg, 48%): LCMS (ESI) calc'd for C₂₁H₃₀Cl₂N₂O₅ [M+H]⁺ 461, 463 (3:2) found 461, 463 (3:2); ¹H NMR (400 MHz, CD₃OD) δ 7.46 (d, J=9.0 Hz, 1H), 7.03 (d, J=9.0 Hz, 1H), 4.24-4.12 (m, 2H), 3.91 (s, 3H), 3.88-3.69 (m, 6H), 3.46-3.36 (m, 3H), 2.95-2.82 (m, 2H), 2.51-2.36 (m, 1H), 2.36-2.33 (m, 1H), 1.53 (s, 9H).

Step b:

To a stirred solution of tert-butyl (2S,4R)-4-(2,3-dichloro-6-hydroxyphenyl)-2-[[(3-methoxy-3-oxopropyl)amino]methyl]pyrrolidine-1-carboxylate (120 mg, 0.26 mmol) in DCM (2 mL) was added TFA (2 mL) at room temperature. The reaction was stirred at room temperature for 1 h. The reaction was concentrated under reduced pressure. The residue was dissolved in MeOH (3 mL) and LiOH. H₂O (33.0 mg, 0.78 mmol) was added. The reaction was stirred at 40° C. for 1 h, and concentrated under reduced pressure. The crude product was dissolved in DMF (3 mL) and HATU (200 mg, 0.52 mmol) added. The resulting solution was stirred for 1 h at room temperature, diluted with water (30 mL) and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 50% ACN in water (plus 0.05% TFA) to afford to afford (8R,9aS)-8-(2,3-dichloro-6-methoxyphenyl)-octahydropyrrolo[1,2-a][1,4]diazepin-5-one as a yellow oil (30.0 mg, 35%): LCMS (ESI) calc'd for C₁₅H₁₈Cl₂N₂O₂ [M+H]⁺ 329, 331 (3:2) found 329, 331 (3:2); ¹H NMR (400 MHz, CD₃OD) δ 7.45 (d, J=9.0 Hz, 1H), 7.02 (d, J=9.0 Hz, 1H), 4.44-4.34 (m, 1H), 4.27-4.14 (m, 1H), 3.92-3.86 (m, 5H), 3.67-3.55 (m, 2H), 3.31-3.16 (m, 2H), 3.16-3.04 (m, 1H), 2.80-2.70 (m, 1H), 2.61-2.49 (m, 1H), 2.46-2.36 (m, 1H).

Step c:

To a stirred solution of (8R,9aS)-8-(2,3-dichloro-6-methoxyphenyl)-octahydropyrrolo[1,2-a][1,4]diazepin-5-one (30.0 mg, 0.09 mmol) in DCM (1 mL) was added BBr₃ (91.0 mg, 0.37 mmol) at room temperature. The reaction was stirred at room temperature for 1 h. The reaction was quenched with MeOH (10 mL) and concentrated under reduced pressure. The residue was purified by reverse phase chromatography, eluting with 20% ACN in water (plus 0.05% TFA) to afford (8R,9aS)-8-(2,3-dichloro-6-hydroxyphenyl)-octahydropyrrolo[1,2-a][1,4]diazepin-5-one trifluoroacetic acid as a colorless oil (30.0 mg, 77%): LCMS (ESI) calc'd for C₁₄H₁₆Cl₂N₂O₂ [M+H]⁺ 315, 317 (3:2) found 315, 317 (3:2); ¹H NMR (400 MHz, CD₃OD) δ 7.32 (d, J=8.8 Hz, 1H), 6.88 (d, J=8.8 Hz, 1H), 4.55-4.42 (m, 1H), 4.05-3.96 (m, 1H), 3.80-3.70 (m, 1H), 3.70-3.43 (m, 4H), 3.31-3.18 (m, 2H), 2.77-2.62 (m, 1H), 2.56-2.41 (m, 1H), 2.36-2.16 (m, 1H).

Step d:

To a stirred solution of glycolic acid (6 mg, 0.08 mmol), HOBT (11.0 mg, 0.08 mmol) and EDCI (16.0 mg, 0.08 mmol) in DMF (1 mL) were added (8R,9aS)-8-(2,3-dichloro-6-hydroxyphenyl)-octahydropyrrolo[1,2-a][1,4]diazepin-5-one trifluoroacetic acid (30.0 mg, 0.07 mmol) and TEA (21.0 mg, 0.21 mmol) at room temperature. The reaction was stirred at room temperature for 2 h. The reaction was quenched with MeOH (0.5 mL) and was purified with Prep-HPLC with the following conditions: Column: XBridge Shield RP18 OBD Column 30×150 mm, 5 m; Mobile Phase A: water (plus 0.05% TFA), Mobile Phase B: ACN; Flow rate: 60 mL/min; Gradient: 15% B to 45% B in 8 min; Detector: UV 254/220 nm; Retentione time: 6.98 min. The fractions containing the desired product were collected and concentrated under reduced pressure to afford Compound 18 (8R,9aS)-8-(2,3-dichloro-6-hydroxyphenyl)-2-(2-hydroxyacetyl)-hexahydro-1H-pyrrolo[1,2-a][1,4]diazepin-5-one as an off-white solid (8.7 mg, 33%): LCMS (ESI) calc'd for C₁₆H₁₈Cl₂N₂O₄ [M+H]⁺ 373, 375 (3:2) found 373, 375 (3:2); ¹H NMR (400 MHz, CD₃OD) δ 7.27 (d, J=8.8 Hz, 1H), 6.77 (d, J=8.8 Hz, 1H), 4.77-4.58 (m, 1H), 4.40-4.24 (m, 2H), 4.17-3.75 (m, 5H), 3.30-2.94 (m, 1H), 2.92-2.78 (m, 1H), 2.78-2.57 (m, 3H), 2.43-2.26 (m, 1H).

### Example 33. Compound 90 ((8R,9aS)-8-(2,3-dichloro-6-hydroxyphenyl)-3-(2-hydroxyacetyl)-hexahydro-1H-pyrrolo[1,2-d][1,4]diazepin-5-one)

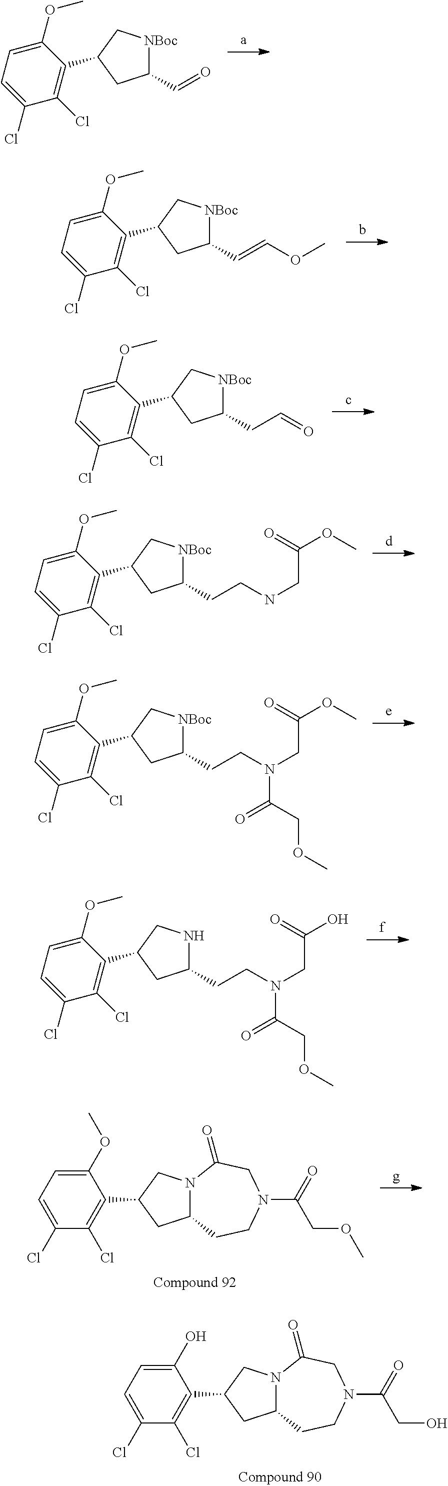

Step a:

To a stirred mixture of (methoxymethyl)triphenylphosphanium chloride (770 mg, 2.23 mmol) in THE (5 mL) was added t-BuOK (2.22 mL, 2.22 mmol, 1 M in THF) dropwise at 0° C. under nitrogen atmosphere. The reaction was stirred at 0° C. for 15 min. Then tert-butyl (2S,4R)-4-(2,3-dichloro-6-methoxyphenyl)-2-formylpyrrolidine-1-carboxylate (Example 7, step c) (420 mg, 1.12 mmol) in THE (1 mL) was added. The reaction was stirred at 0° C. for 1 h then diluted with EA (30 mL) and water (30 mL). The aqueous solution was extracted with EA (3×20 mL). The combined organic layers were washed with brine (3×30 mL) and dried over anhydrous Na₂SO₄. After filtration, the filtrate was concentrated under reduced pressure. The residue was purified by reverse phase chromatography, eluting with 70% ACN in water (plus 0.05% TFA) to afford tert-butyl (2S,4R)-4-(2,3-dichloro-6-methoxyphenyl)-2-(2-methoxyethenyl)pyrrolidine-1-carboxylate as a yellow oil (300 mg, 59%): LCMS (ESI) calc'd for C₁₉H₂₅Cl₂NO₄ [M+H]⁺ 402, 404 (3:2) found 402, 404 (3:2); ¹H NMR (400 MHz, CD₃OD) δ 7.47-7.39 (m, 1H), 7.03-6.95 (m, 1H), 4.85-4.58 (m, 1H), 4.58-4.20 (m, 1H), 4.14-3.68 (m, 4H), 3.66-3.46 (m, 4H), 2.91-2.16 (m, 2H), 1.87-1.61 (m, 2H), 1.57-1.43 (m, 9H).

Step b:

To a stirred solution of tert-butyl (2S,4R)-4-(2,3-dichloro-6-methoxyphenyl)-2-(2-methoxyethenyl)pyrrolidine-1-carboxylate (300 mg, 0.75 mmol) in acetone (5 mL) were added TsOH.H₂O (71.0 mg, 0.37 mmol) at room temperature. The reaction was stirred at room temperature for 0.5 h. The reaction was diluted with water (20 mL). The aqueous solution was extracted with EA (2×30 mL). The combined organic layers were washed brine (2×30 mL) and dried over anhydrous Na₂SO₄. After filtration, the filtrate was concentrated under reduced pressure to afford tert-butyl (2S,4R)-4-(2,3-dichloro-6-methoxyphenyl)-2-(2-oxoethyl)pyrrolidine-1-carboxylate as a yellow oil (300 mg, crude), which was used directly in the next step without further purification: LCMS (ESI) calc'd for C₁₈H₂₃Cl₂NO₄ [M+H]⁺ 388, 340 (3:2) found 388, 340 (3:2);

Step c:

To a stirred solution of tert-butyl (2S,4R)-4-(2,3-dichloro-6-methoxyphenyl)-2-(2-oxoethyl)pyrrolidine-1-carboxylate (210 mg, 0.54 mmol) and methyl 2-aminoacetate hydrochloride (140 mg, 1.08 mmol) in DCM (2 mL) were added TEA (160 mg, 1.62 mmol) and NaBH(AcO)₃ (340 mg, 1.62 mmol) at room temperature. The reaction was stirred at room temperature for 1 h, diluted with water (20 mL) and extracted with EA (2×30 mL). The combined organic layers were washed brine (2×30 mL) and dried over anhydrous Na₂SO₄. After filtration, the filtrate was concentrated under reduced pressure. The residue was purified by reverse phase chromatography, eluting with 35% ACN in water (plus 0.05% TFA) to afford tert-butyl (2S,4R)-4-(2,3-dichloro-6-methoxyphenyl)-2-[2-[(2-methoxy-2-oxoethyl)amino]ethyl]pyrrolidine-1-carboxylate as a light yellow oil (110 mg, 48% over two steps): LCMS (ESI) calc'd for C₂₁H₃₀Cl₂N₂O₅ [M+H]⁺ 461, 463 (3:2) found 461, 463 (3:2);

Step d:

To a stirred solution of methoxyacetic acid (43.0 mg, 0.48 mmol) and HATU (180 mg, 0.48 mmol) in DMF (2 mL) were added [2-[(2S,4R)-1-(tert-butoxycarbonyl)-4-(2,3-dichloro-6-methoxyphenyl)pyrrolidin-2-yl]ethyl](2-methoxy-2-oxoethyl)aminyl (110 mg, 0.24 mmol) and TEA (72.0 mg, 0.72 mmol) at room temperature. The reaction was stirred at room temperature for 1 h. The reaction was purified by reverse phase chromatography, eluting with 40% ACN in water (plus 0.05% TFA) to afford tert-butyl (2S,4R)-4-(2,3-dichloro-6-methoxyphenyl)-2-[2-[2-methoxy-N-(2-methoxy-2-oxoethyl)acetamido]ethyl]pyrrolidine-1-carboxylate as a light yellow oil (50.0 mg, 39%): LCMS (ESI) calc'd for C₂₄H₃₄Cl₂N₂O₇ [M+H]⁺ 533, 535 (3:2) found 533, 535 (3:2); ¹H NMR (400 MHz, CD₃OD) δ 7.43 (d, J=8.9 Hz, 1H), 7.00 (d, J=9.1 Hz, 1H), 4.44-3.98 (m, 6H), 3.95-3.70 (m, 8H), 3.55-3.41 (m, 4H), 2.69-2.57 (m, 1H), 2.10-1.63 (m, 4H), 1.58-1.46 (m, 9H).

Step e:

To a stirred solution of tert-butyl (2S,4R)-4-(2,3-dichloro-6-methoxyphenyl)-2-[2-[2-methoxy-N-(2-methoxy-2-oxoethyl)acetamido]ethyl]pyrrolidine-1-carboxylate (50.0 mg, 0.09 mmol) in DCM (2 mL) was added TFA (1 mL) at room temperature. The reaction was stirred at room temperature for 1 h. The reaction was concentrated under reduced pressure. The residue was dissolved in MeOH (2 mL) and LiOH.H₂O (20.0 mg, 0.47 mmol) in water (0.5 mL) was added. The reaction was stirred at 40° C. for 1 h. The reaction was concentrated under reduced pressure to afford (N-[2-[(2S,4R)-4-(2,3-dichloro-6-methoxyphenyl)pyrrolidin-2-yl]ethyl]-2-methoxyacetamido)acetic acid as a yellow solid (50.0 mg, crude), which was used directly in the next step without further purification: LCMS (ESI) calc'd for C₁₈H₂₄Cl₂N₂O₅ [M+H]⁺ 419, 421 (3:2) found 419, 421 (3:2).

Step f:

A solution of (N-[2-[(2S,4R)-4-(2,3-dichloro-6-methoxyphenyl)pyrrolidin-2-yl]ethyl]-2-methoxyacetamido)acetic acid (50.0 mg, 0.12 mmol) and HATU (45.0 mg, 0.12 mmol) in DMF (0.50 mL) was stirred at room temperature for 1 h. The reaction was quenched with water (0.2 mL). The reaction solution was purified by reverse phase chromatography, eluting with 35% ACN in water (plus 0.05% TFA) to afford (8R,9aS)-8-(2,3-dichloro-6-methoxyphenyl)-3-(2-methoxyacetyl)-hexahydro-1H-pyrrolo[1,2-d][1,4]diazepin-5-one as a light yellow oil (25.0 mg, 65% over two steps): LCMS (ESI) calc'd for C₁₈H₂₂Cl₂N₂O₄ [M+H]⁺ 401, 403 (3:2) found 401, 403 (3:2); ¹H NMR (400 MHz, CD₃OD) δ 7.43 (d, J=9.0 Hz, 1H), 7.00 (d, J=9.0 Hz, 1H), 4.45-3.98 (m, 6H), 3.97-3.67 (m, 7H), 3.60-3.37 (m, 4H), 2.10-1.86 (m, 2H), 1.82-1.67 (m, 1H).

Step g:

To a stirred solution of (8R,9aS)-8-(2,3-dichloro-6-methoxyphenyl)-3-(2-methoxyacetyl)-hexahydro-1H-pyrrolo[1,2-d][1,4]diazepin-5-one (25.0 mg, 0.06 mmol) in DCM (1 mL) was added BBr₃ (94.0 mg, 0.37 mmol) at room temperature. The reaction was stirred at room temperature for 1 h. The reaction was quenched with MeOH (1 mL) and concentrated under reduced pressure. The residue was purified by Prep-HPLC with the following conditions: Column: Xselect CSH OBD Column 30×150 mm 5 μm; Mobile Phase A: Water (plus 0.05% TFA), Mobile Phase B: ACN; Flow rate: 60 mL/min; Gradient: 15% B to 45% B in 7 min; Detector: UV 220 nm; Retention Time: 6.92 min. The fractions containing the desired product were combined and concentrated under reduced pressure to afford Compound 90 ((8R,9aS)-8-(2,3-dichloro-6-hydroxyphenyl)-3-(2-hydroxyacetyl)-hexahydro-1H-pyrrolo[1,2-d][1,4]diazepin-5-one) as an off-white solid (7.8 mg, 34%): LCMS (ESI) calc'd for C₁₆H₁₈Cl₂N₂O₄ [M+H]⁺ 373, 375 (3:2) found 373, 375 (3:2); ¹H NMR (400 MHz, CD₃OD) δ 7.25 (d, J=8.8 Hz, 1H), 6.75 (d, J=8.8 Hz, 1H), 4.47-3.97 (m, 8H), 3.86-3.67 (m, 2H), 3.10-2.65 (m, 1H), 2.35-1.85 (m, 3H).

### Example 34. Compound 91 ((8R,9aS)-8-(2,3-dichloro-6-hydroxyphenyl)-2-(2-hydroxyacetyl)-4-methyloctahydro-5H-pyrrolo[1,2-a][1,4]diazepin-5-one isomer 1) and Compound 92 ((8R,9aS)-8-(2,3-dichloro-6-hydroxyphenyl)-2-(2-hydroxyacetyl)-4-methyloctahydro-5H-pyrrolo[1,2-a][1,4]diazepin-5-one isomer 2)

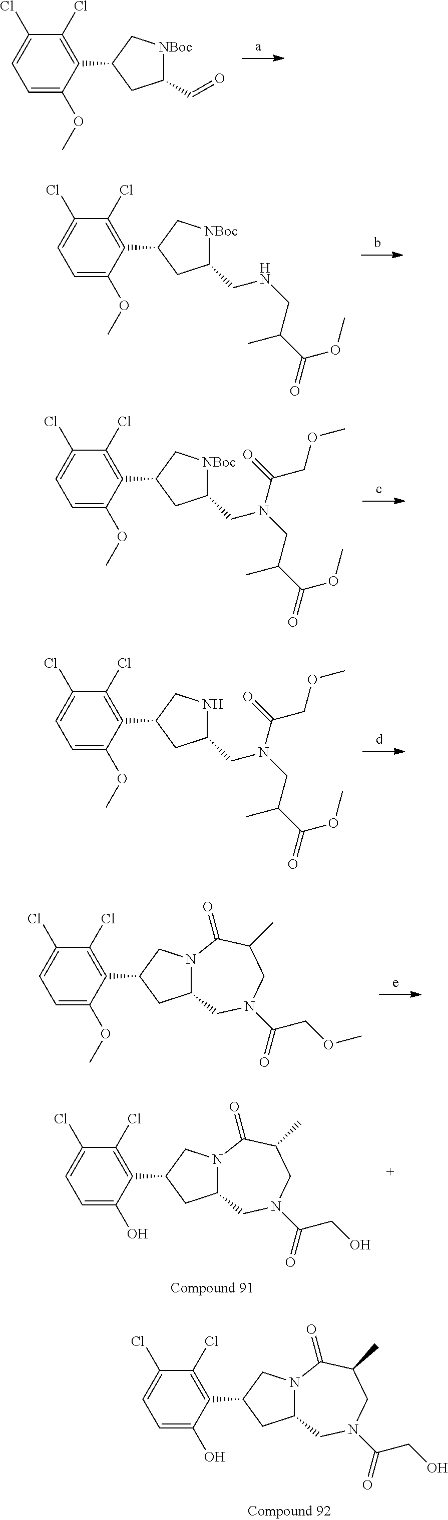

Step a:

To a stirred solution of tert-butyl (2S,4R)-4-(2,3-dichloro-6-methoxyphenyl)-2-formylpyrrolidine-1-carboxylate (Example 7, step c) (300 mg, 0.80 mmol) and methyl 3-amino-2-methylpropanoate (110 mg, 0.96 mmol) in DCM (4 mL) were added NaOAc (130 mg, 1.60 mmol) and NaBH(OAc)₃ (500 mg, 2.40 mmol) at room temperature. The resulting mixture was stirred for 1 h at room temperature. The resulting mixture was quenched with saturated aq. NH₄Cl (30 mL) followed by extraction with EA (3×20 mL). The combined organic phases were washed with brine (2×20 mL) and dried over anhydrous Na₂SO₄. After filtration, the filtrate was concentrated under reduced pressure. The residue was purified by reverse phase chromatography, eluting with 55% ACN in water (plus 0.05% TFA) to afford tert-butyl (2S,4R)-4-(2,3-dichloro-6-methoxyphenyl)-2-[[(3-methoxy-2-methyl-3-oxopropyl)amino]methyl]pyrrolidine-1-carboxylate as a light yellow oil (400 mg, 80%): LCMS (ESI) calc'd for C₂₂H₃₂Cl₂N₂O₅ [M+H]⁺: 475, 477 (3:2) found 475, 477 (3:2); ¹H NMR (400 MHz, CDCl₃) δ 7.36 (d, J=8.9 Hz, 1H), 6.78 (d, J=9.0 Hz, 1H), 4.26-4.08 (m, 1H), 3.86 (s, 3H), 3.83-3.72 (m, 4H), 3.67 (t, J=9.6 Hz, 1H), 3.54-3.38 (m, 2H), 3.25-3.00 (m, 4H), 2.48-2.24 (m, 2H), 1.49 (d, J=3.5 Hz, 9H), 1.38-1.29 (m, 3H).

Step b:

To a stirred solution of methoxyacetic acid (110 mg, 1.26 mmol) and HATU (480 mg, 1.26 mmol) in DMF (4 mL) were added tert-butyl (2S,4R)-4-(2,3-dichloro-6-methoxyphenyl)-2-[[(3-methoxy-2-methyl-3-oxopropyl)amino]methyl]pyrrolidine-1-carboxylate (400 mg, 0.84 mmol) and TEA (250 mg, 2.52 mmol) at room temperature. The resulting mixture was stirred for 2 h at room temperature, diluted with water (30 mL) and extracted with EA (3×20 mL). The combined organic phases were washed with brine (2×30 mL) and dried over anhydrous Na₂SO₄. After filtration, the filtrate was concentrated under reduced pressure. The residue was purified by reverse phase chromatography, eluting with 60% ACN in water (plus 0.05% TFA) to afford tert-butyl (2S,4R)-4-(2,3-dichloro-6-methoxyphenyl)-2-[[2-methoxy-N-(3-methoxy-2-methyl-3-oxopropyl)acetamido]methyl]pyrrolidine-1-carboxylate as a yellow oil (300 mg, 65%): LCMS (ESI) calc'd for C₂₅H₃₆Cl₂N₂O₇ [M+H]⁺: 547, 549 (3:2) found 547, 549 (3:2); ¹H NMR (400 MHz, CDCl₃) δ 7.34 (d, J=8.9 Hz, 1H), 6.78 (d, J=8.9 Hz, 1H), 4.31-4.12 (m, 4H), 4.12-3.93 (m, 1H), 3.90 (d, J=4.1 Hz, 3H), 3.78-3.60 (m, 6H), 3.43 (s, 3H), 3.13-2.83 (m, 3H), 2.39-2.15 (m, 2H), 1.50 (d, J=19.1 Hz, 9H), 1.25-1.10 (m, 3H).

Step c:

To a stirred solution of tert-butyl (2S,4R)-4-(2,3-dichloro-6-methoxyphenyl)-2-[[2-methoxy-N-(3-methoxy-2-methyl-3-oxopropyl)acetamido]methyl]pyrrolidine-1-carboxylate (300 mg, 0.55 mmol) in DCM (3 mL) was added TFA (1.5 mL) at room temperature. The resulting mixture was stirred for 1 h at room temperature. The resulting solution was concentrated under reduced pressure to afford methyl 3-(N-[[(2S,4R)-4-(2,3-dichloro-6-methoxyphenyl)pyrrolidin-2-yl]methyl]-2-methoxyacetamido)-2-methylpropanoate as a yellow oil (0.30 g, crude), which was used directly in the next step without further purification: LCMS (ESI) calc'd for C₂₀H₂₈Cl₂N₂O₅ [M+H]⁺: 447, 449 (3:2) found 447,449 (3:2).

Step d:

To a stirred solution of methyl 3-(N-[[(2S,4R)-4-(2,3-dichloro-6-methoxyphenyl)pyrrolidin-2-yl]methyl]-2-methoxyacetamido)-2-methylpropanoate (300 mg, 0.67 mmol) in MeOH (3 mL) was added LiOH H₂O (56.0 mg, 1.34 mmol) in H₂O (1 mL) at room temperature. The resulting mixture was stirred for 1 h at 40° C. The resulting mixture was concentrated under reduced pressure. The crude product was dissolved with DMF (3 mL) and HATU (380 mg, 1.00 mmol) added. The reaction mixture was stirred for 1 h at room temperature, diluted with water (30 mL) and extracted with EA (3×20 mL). The combined organic phases were washed with brine (2×30 mL) and dried over anhydrous Na₂SO₄. After filtration, the filtrate was concentrated under reduced pressure. The residue was purified by reverse phase chromatography, eluting with 30% ACN in water (plus 0.05% TFA) to afford (8R,9aS)-8-(2,3-dichloro-6-methoxyphenyl)-2-(2-methoxyacetyl)-4-methyl-hexahydro-1H-pyrrolo[1,2-a][1,4]diazepin-5-one as a yellow oil (60.0 mg, 27% over two steps): LCMS (ESI) calc'd for C₁₉H₂₄Cl₂N₂O₄ [M+H]⁺: 415, 417 (3:2) found 415, 417 (3:2); ¹H NMR (400 MHz, CDCl₃) δ 7.41-7.35 (m, 1H), 6.83-6.77 (m, 1H), 4.49-4.35 (m, 1H), 4.34-4.03 (m, 3H), 4.01-3.82 (m, 4H), 3.82-3.50 (m, 4H), 3.47 (s, 3H), 3.33-2.95 (m, 2H), 2.59-2.18 (m, 2H), 1.39-1.27 (m, 3H).

Step e:

To a stirred solution of (8R,9aS)-8-(2,3-dichloro-6-methoxyphenyl)-2-(2-methoxyacetyl)-4-methyl-hexahydro-1H-pyrrolo[1,2-a][1,4]diazepin-5-one (60.0 mg, 0.14 mmol) in DCM (1 mL) was added BBr₃ (0.5 mL) at room temperature. The resulting mixture was stirred for 1 h at room temperature. The reaction was quenched with MeOH (2 mL) at room temperature. The resulting mixture was concentrated under reduced pressure. The residue was purified by Prep-HPLC with the following conditions: Column: Xselect CSH OBD Column 30×150 mm 5 μm; Mobile Phase A: Water (plus 0.05% TFA), Mobile Phase B: ACN; Flow rate: 60 mL/min; Gradient: 10% to 40% in 8 min; Detector: UV 254/220 nm; Retention time 1: 8.68 min, Retention time 2: 8.98 min. The fractions containing the desired product at 8.68 min were collected and concentrated under reduced pressure to afford Compound 91 ((8R,9aS)-8-(2,3-dichloro-6-hydroxyphenyl)-2-(2-hydroxyacetyl)-4-methyl-hexahydro-1H-pyrrolo[1,2-a][1,4]diazepin-5-one isomer 1) as an off-white solid (3.8 mg, 2%): LCMS (ESI) calc'd for C₁₇H₂₀Cl₂N₂O₄ [M+H]⁺: 387, 389 (3:2) found 387, 389 (3:2); ¹H NMR (400 MHz, CD₃OD) δ 7.27 (d, J=8.8 Hz, 1H), 6.77 (d, J=8.8 Hz, 1H), 4.65-4.36 (m, 1H), 4.36-4.29 (m, 2H), 4.29-4.10 (m, 2H), 4.09-3.98 (m, 2H), 3.82-3.51 (m, 2H), 3.29-3.18 (m, 1H), 3.00-2.87 (m, 1H), 2.76-2.63 (m, 1H), 2.30 (dt, J=12.8, 6.6 Hz, 1H), 1.29 (dd, J=18.8, 7.5 Hz, 3H). The fractions containing the desired product at 8.98 min were collected and concentrated under reduced pressure to afford Compound 92 ((8R,9aS)-8-(2,3-dichloro-6-hydroxyphenyl)-2-(2-hydroxyacetyl)-4-methyl-hexahydro-1H-pyrrolo[1,2-a][1,4]diazepin-5-one isomer 2) as an off-white solid. (2 mg, 1%) LCMS (ESI) calc'd for C₁₇H₂₀Cl₂N₂O₄ [M+H]⁺: 387, 389 (3:2) found 387, 389 (3:2); ¹H NMR (400 MHz, CD₃OD) δ 7.27 (d, J=8.8 Hz, 1H), 6.77 (d, J=8.8 Hz, 1H), 4.71-4.27 (m, 3H), 4.19-3.97 (m, 3H), 3.98-3.57 (m, 2H), 3.23-3.10 (m, 1H), 2.88-2.63 (m, 3H), 2.42-2.29 (m, 1H), 1.21 (d, J=7.0 Hz, 3H).

### Example 35. Compound 93 ((3R,8R,9aS)-8-(2,3-dichloro-6-hydroxyphenyl)-2-(2-hydroxyacetyl)-3-methyl-hexahydro-1H-pyrrolo[1,2-a][1,4]diazepin-5-one)

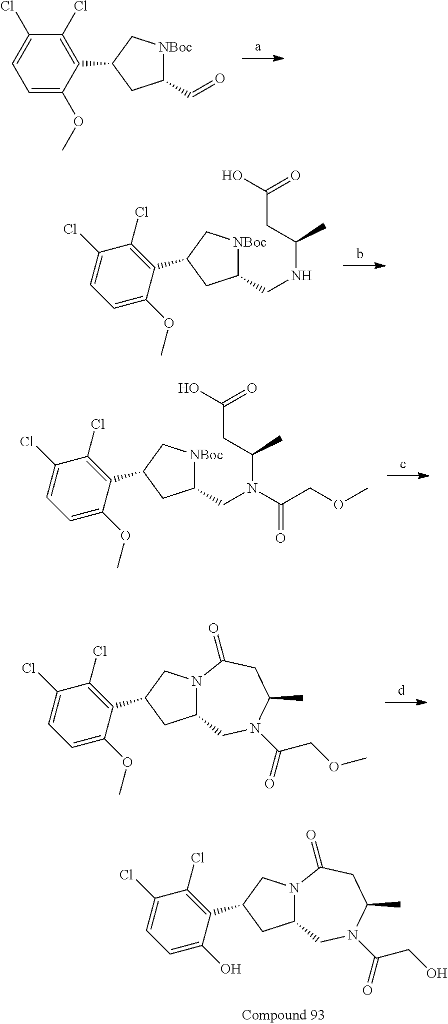 [SMILES: COc1ccc(Cl)c(Cl)c1[C@H]1C[C@@H](C=O)N(C(=O)OC(C)(C)C)C1 ; COc1ccc(Cl)c(Cl)c1[C@H]1C[C@@H](CN[C@H](C)CC(=O)O)N(C(=O)OC(C)(C)C)C1 ; COCC(=O)N(C[C@@H]1C[C@H](c2c(OC)ccc(Cl)c2Cl)CN1C(=O)OC(C)(C)C)[C@H](C)CC(=O)O ; COCC(=O)N1C[C@@H]2C[C@H](c3c(OC)ccc(Cl)c3Cl)CN2C(=O)C[C@H]1C ; C[C@@H]1CC(=O)N2C[C@@H](c3c(O)ccc(Cl)c3Cl)C[C@H]2CN1C(=O)CO]

Step a:

To a stirred solution of tert-butyl (2S,4R)-4-(2,3-dichloro-6-methoxyphenyl)-2-formylpyrrolidine-1-carboxylate (Example 7, step c) (400 mg, 1.07 mmol) and (3R)-3-aminobutanoic acid (170 mg, 1.60 mmol) in DCM (5 mL) were added HOAc (0.06 mL, 1.020 mmol) and NaBH(AcO)₃ (680 mg, 3.21 mmol) at room temperature. The reaction was stirred at room temperature for 1 h. The resulting mixture was extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 50% ACN in water (plus 0.05% TFA) to afford (3R)-3-([[(2S,4R)-1-(tert-butoxycarbonyl)-4-(2,3-dichloro-6-methoxyphenyl)pyrrolidin-2-yl]methyl]amino)butanoic acid as a light yellow oil (300 mg, 55%): LCMS (ESI) calc'd for C₂₁H₃₀Cl₂N₂O₅ [M+H]⁺: 461, 463 (3:2) found 461, 463 (3:2); ¹H NMR (400 MHz, CDCl₃) δ 7.35 (d, J=8.9 Hz, 1H), 6.78 (d, J=9.0 Hz, 1H), 4.23-4.00 (m, 2H), 3.87 (s, 3H), 3.81-3.69 (m, 2H), 3.32-3.23 (m, 1H), 3.20 (d, J=11.7 Hz, 1H), 3.04-2.94 (m, 1H), 2.63-2.43 (m, 2H), 2.38-2.22 (m, 2H), 1.49 (s, 9H), 1.34 (d, J=6.6 Hz, 3H).

Step b:

To a stirred solution of methoxyacetic acid (79.0 mg, 0.88 mmol) and HATU (300 mg, 0.88 mmol) in DMF (3 mL) were added (3R)-3-([[(2S,4R)-1-(tert-butoxycarbonyl)-4-(2,3-dichloro-6-methoxyphenyl)pyrrolidin-2-yl]methyl]amino)butanoic acid (270 mg, 0.59 mmol) and TEA (0.24 mL, 2.41 mmol) at room temperature. The resulting mixture was stirred for 1 h at room temperature, diluted with water (50 mL) and extracted with EA (3×20 mL). The combined organic phases were washed with brine (2×50 mL) and dried over anhydrous Na₂SO₄. After filtration, the filtrate was concentrated under reduced pressure. The residue was purified by reverse phase chromatography, eluting with 50% ACN in water (plus 0.05% TFA) to afford (3R)-3-(N-[[(2S,4R)-1-(tert-butoxycarbonyl)-4-(2,3-dichloro-6-methoxyphenyl)pyrrolidin-2-yl]methyl]-2-methoxyacetamido)butanoic acid as a light yellow oil (220 mg, 63%): LCMS (ESI) calc'd for C₂₄H₃₄Cl₂N₂O₇ [M+H]⁺: 533, 535 (3:2) found 533, 535 (3:2); ¹H NMR (400 MHz, CDCl₃) δ 7.33 (dd, J=20.6, 10.4 Hz, 1H), 6.77 (dd, J=16.2, 9.2 Hz, 1H), 4.40-4.01 (m, 3H), 4.00-3.83 (m, 4H), 3.84-3.63 (m, 3H), 3.59-3.37 (m, 4H), 3.10-2.86 (m, 1H), 2.63-2.40 (m, 2H), 2.34-2.07 (m, 2H), 1.58-1.43 (m, 9H), 1.35-1.26 (m, 3H).

Step c:

To a stirred solution of (3R)-3-(N-[[(2S,4R)-1-(tert-butoxycarbonyl)-4-(2,3-dichloro-6-methoxyphenyl)pyrrolidin-2-yl]methyl]-2-methoxyacetamido)butanoic acid (220 mg, 0.41 mmol) in DCM (2 mL) was added TFA (0.50 mL) at room temperature. The reaction was stirred for 1 h and concentrated under reduced pressure. The residue was dissolved in DMF (2 mL) and TEA (130 mg, 1.237 mmol) and HATU (240 mg, 0.619 mmol) were added sequentially at room temperature. The resulting mixture was stirred for 1 h, diluted with water (80 mL) and extracted with EA (3×20 mL). The combined organic phases were washed with brine (2×50 mL) and dried over anhydrous Na₂SO₄. After filtration, the filtrate was concentrated under reduced pressure. The residue was purified by reverse phase chromatography, eluting with 48% ACN in water (plus 0.05% TFA) to afford (3R,8R,9aS)-8-(2,3-dichloro-6-methoxyphenyl)-2-(2-methoxyacetyl)-3-methyl-hexahydro-1H-pyrrolo[1,2-a][1,4]diazepin-5-one as a light yellow oil (140 mg, 74%): LCMS (ESI) calc'd for C₁₉H₂₄Cl₂N₂O₄ [M+H]⁺: 415, 417 (3:2) found 415, 417 (3:2).

Step d:

To a stirred mixture of (3R,8R,9aS)-8-(2,3-dichloro-6-methoxyphenyl)-2-(2-methoxyacetyl)-3-methyl-hexahydro-1H-pyrrolo[1,2-a][1,4]diazepin-5-one (70.0 mg, 0.17 mmol) in DCM (1 mL) was added BBr₃ (0.25 mL) dropwise at room temperature. The resulting mixture was stirred for 1 h at room temperature then quenched with MeOH (5 mL) at 0° C. and concentrated under reduced pressure. The residue was purified by Prep-HPLC with the following conditions: Column: XBridge Shield RP18 OBD Column, 30×150 mm, 5 m; Mobile Phase A: water (0.05% TFA), Mobile Phase B: ACN; Flow rate: 60 mL/min; Gradient: 20% B to 40% B in 7.00 min; Detector: UV 220 nm; Retention time: 7.03 min. The fractions containing the desired product were collected and concentrated under reduced pressure to afford Compound 93 ((3R,8R,9aS)-8-(2,3-dichloro-6-hydroxyphenyl)-2-(2-hydroxyacetyl)-3-methyl-hexahydro-1H-pyrrolo[1,2-a][1,4]diazepin-5-one) as an off-white solid (19.7 mg, 29%): LCMS (ESI) calc'd for C₁₇H₂₀Cl₂N₂O₄ [M+H]⁺: 387, 389 (3:2) found 387, 389 (3:2); ¹H NMR (400 MHz, CD₃OD) δ 7.25 (d, J=8.6 Hz, 1H), 6.75 (d, J=8.6 Hz, 1H), 4.83-4.69 (m, 1H), 4.43-4.06 (m, 5H), 4.06-3.92 (m, 1H), 3.81-3.52 (m, 2H), 3.12-2.71 (m, 2H), 2.69-2.45 (m, 1H), 2.26 (d, J=54.0 Hz, 1H), 1.36-1.22 (m, 3H).

### Example 36. Compound 94 ((3S,8R,9aS)-8-(2,3-dichloro-6-hydroxyphenyl)-2-(2-hydroxyacetyl)-3-methyl-hexahydro-1H-pyrrolo[1,2-a][1,4]diazepin-5-one)

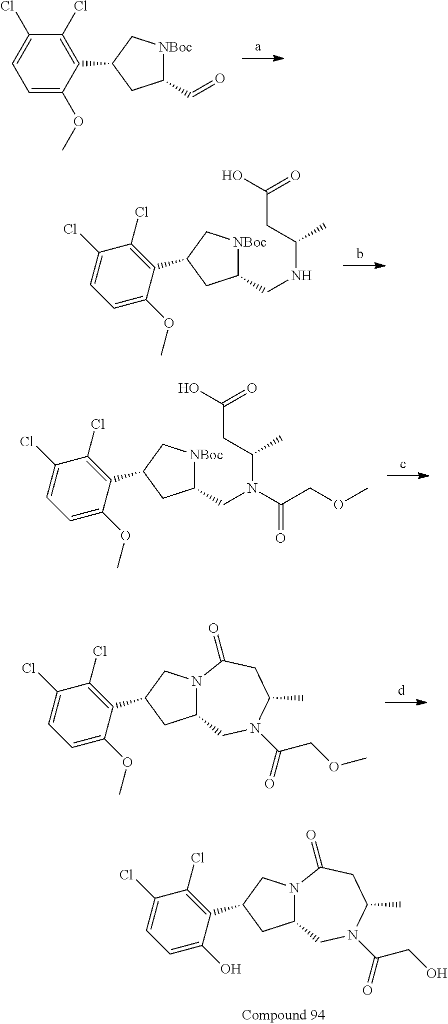 [SMILES: COc1ccc(Cl)c(Cl)c1[C@H]1C[C@@H](C=O)N(C(=O)OC(C)(C)C)C1 ; COc1ccc(Cl)c(Cl)c1[C@H]1C[C@@H](CN[C@@H](C)CC(=O)O)N(C(=O)OC(C)(C)C)C1 ; COCC(=O)N(C[C@@H]1C[C@H](c2c(OC)ccc(Cl)c2Cl)CN1C(=O)OC(C)(C)C)[C@@H](C)CC(=O)O ; COCC(=O)N1C[C@@H]2C[C@H](c3c(OC)ccc(Cl)c3Cl)CN2C(=O)C[C@@H]1C ; C[C@H]1CC(=O)N2C[C@@H](c3c(O)ccc(Cl)c3Cl)C[C@H]2CN1C(=O)CO]

Step a:

To a stirred solution of tert-butyl (2S,4R)-4-(2,3-dichloro-6-methoxyphenyl)-2-formylpyrrolidine-1-carboxylate (Example 7, step c) (400 mg, 1.07 mmol) and (3S)-3-aminobutanoic acid (170 mg, 1.60 mmol) in DCM (5 mL) were added HOAc (0.06 mL, 1.02 mmol) and NaBH(AcO)₃ (680 mg, 3.21 mmol) at room temperature. The reaction was stirred at room temperature for 1 h. The reaction was quenched with saturated aq. NH₄Cl (20 mL) followed by extraction with EA (3×30 mL). The combined organic layers were washed with brine (2×20 mL) and dried over anhydrous Na₂SO₄. After filtration, the filtrate was concentrated under reduced pressure. The residue was purified by reverse phase chromatography, eluting with 50% ACN in water (plus 0.05% TFA) to afford (3S)-3-([[(2S,4R)-1-(tert-butoxycarbonyl)-4-(2,3-dichloro-6-methoxyphenyl)pyrrolidin-2-yl]methyl]amino)butanoic acid as a light yellow oil (220 mg, 40%): LCMS (ESI) calc'd for C₂₁H₃₀Cl₂N₂O₅ [M+H]⁺: 461, 463 (3:2) found 461, 463 (3:2); ¹H NMR (400 MHz, CDCl₃) δ 7.36 (d, J=8.9 Hz, 1H), 6.78 (d, J=9.0 Hz, 1H), 4.18-4.02 (m, 2H), 3.87 (s, 3H), 3.83-3.66 (m, 2H), 3.18-2.93 (m, 2H), 2.93-2.80 (m, 1H), 2.62-2.51 (m, 2H), 2.44-2.26 (m, 2H), 1.48 (s, 9H), 1.33 (d, J=6.5 Hz, 3H).

Step b:

To a stirred solution of methoxyacetic acid (64.0 mg, 0.72 mmol) and HATU (280 mg, 0.72 mmol) in DMF (3 mL) were added (3S)-3-([[(2S,4R)-1-(tert-butoxycarbonyl)-4-(2,3-dichloro-6-methoxyphenyl)pyrrolidin-2-yl]methyl]amino)butanoic acid (220 mg, 0.48 mmol) and TEA (140 mg, 1.43 mmol) at room temperature. The resulting mixture was stirred for 1 h at room temperature, diluted with water (20 mL) and extracted with EA (3×30 mL). The combined organic layers were washed with brine (2×20 mL) and dried over anhydrous Na₂SO₄. After filtration, the filtrate was concentrated under reduced pressure. The residue was purified by reverse phase chromatography, eluting with 50% ACN in water (plus 0.05% TFA) to afford (3S)-3-(N-[[(2S,4R)-1-(tert-butoxycarbonyl)-4-(2,3-dichloro-6-methoxyphenyl)pyrrolidin-2-yl]methyl]-2-methoxyacetamido)butanoic acid as a light yellow oil (140 mg, 50%): LCMS (ESI) calc'd for C₂₄H₃₄Cl₂N₂O₇ [M+H]⁺: 533, 535 (3:2) found 533, 535 (3:2); ¹H NMR (400 MHz, CDCl₃) δ 7.39-7.31 (m, 1H), 6.82-6.73 (m, 1H), 4.38-4.28 (m, 1H), 4.28-4.13 (m, 2H), 4.00-3.86 (m, 4H), 3.86-3.60 (m, 3H), 3.58-3.37 (m, 4H), 3.20-3.09 (m, 1H), 2.63-2.16 (m, 4H), 1.50 (s, 9H), 1.35-1.26 (m, 3H).

Step c:

To a stirred solution of (3S)-3-(N-[[(2S,4R)-1-(tert-butoxycarbonyl)-4-(2,3-dichloro-6-methoxyphenyl)pyrrolidin-2-yl]methyl]-2-methoxyacetamido)butanoic acid (140 mg, 0.26 mmol) in DCM (2 mL) was added TFA (0.5 mL) at room temperature. The reaction was stirred for 1 h and concentrated under reduced pressure. The residue was dissolved in DMF (2 mL) and TEA (0.11 mL, 1.082 mmol) and HATU (150 mg, 0.394 mmol) was added sequentially at room temperature. The resulting reaction was stirred for 1 h, diluted with water (50 mL) and extracted with EA (3×20 mL). The combined organic phases were washed with brine (2×50 mL) and dried over anhydrous Na₂SO₄. After filtration, the filtrate was concentrated under reduced pressure. The residue was purified by reverse phase chromatography, eluting with 45% ACN in water (plus 0.05% TFA) to afford (3S,8R,9aS)-8-(2,3-dichloro-6-methoxyphenyl)-2-(2-methoxyacetyl)-3-methyl-hexahydro-1H-pyrrolo[1,2-a][1,4]diazepin-5-one as a light yellow oil (70.0 mg, 58%): LCMS (ESI) calc'd for C₁₉H₂₄Cl₂N₂O₄ [M+H]⁺: 415, 417 (3:2) found 415, 417 (3:2).

Step d:

To a stirred mixture of (3S,8R,9aS)-8-(2,3-dichloro-6-methoxyphenyl)-2-(2-methoxyacetyl)-3-methyl-hexahydro-1H-pyrrolo[1,2-a][1,4]diazepin-5-one (70.0 mg, 0.17 mmol) in DCM (1 mL) was added BBr₃ (0.25 mL) dropwise at room temperature. The resulting mixture was stirred for 1 h under nitrogen. The reaction was quenched with MeOH (5 mL) at 0° C. The resulting mixture was concentrated under reduced pressure. The residue was purified by Prep-HPLC with the following conditions: Column: XBridge Shield RP18 OBD Column, 30×150 mm, 5 m; Mobile Phase A: water (0.05% TFA), Mobile Phase B: ACN; Flow rate: 60 mL/min; Gradient: 20% B to 40% B in 7 min; Detector: UV 220 nm; Retention time: 7.03 min. The fractions containing the desired product were collected and concentrated under reduced pressure to afford Compound 94 ((3S,8R,9aS)-8-(2,3-dichloro-6-hydroxyphenyl)-2-(2-hydroxyacetyl)-3-methyl-hexahydro-1H-pyrrolo[1,2-a][1,4]diazepin-5-one) as an off-white solid (17.7 mg, 27%): LCMS (ESI) calc'd for C₁₇H₂₀Cl₂N₂O₄ [M+H]⁺: 387, 389 (3:2) found 387, 389 (3:2); ¹H NMR (400 MHz, CD₃OD) δ 7.27 (d, J=8.8 Hz, 1H), 6.78 (d, J=8.8 Hz, 1H), 4.70-4.57 (m, 1H), 4.41-4.25 (m, 2H), 4.26-3.69 (m, 5H), 3.09-2.90 (m, 2H), 2.78-2.53 (m, 2H), 2.46-2.28 (m, 1H), 1.36-1.24 (m, 3H).

### Example 37. Compound 55 ((7R,8aS)-7-(2,3-dichloro-6-hydroxyphenyl)-4-oxo-hexahydropyrrolo[1,2-a]pyrazine-2-carboxamide)

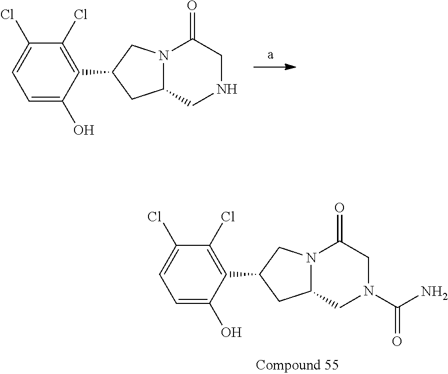 [SMILES: O=C1CNC[C@@H]2C[C@H](c3c(O)ccc(Cl)c3Cl)CN12 ; NC(=O)N1CC(=O)N2C[C@@H](c3c(O)ccc(Cl)c3Cl)C[C@H]2C1]

Step a:

To a stirred solution of (7R,8aS)-7-(2,3-dichloro-6-hydroxyphenyl)-hexahydro-1H-pyrrolo[1,2-a]pyrazin-4-one (Intermediate 8 free base, Example 7) (30.0 mg, 0.10 mmol) and TEA (30.0 mg, 0.29 mmol) in DCM (1 mL) was added isocyanatotrimethylsilane (17 mg, 0.14 mmol) at 0° C. The reaction was stirred for 16 h at room temperature and concentrated under reduced pressure. The residue was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 18% B to 38% B in 8 min; Detector: UV 220 nm; Retention time: 6.40 min. The fractions containing the desired product were collected and concentrated under reduced pressure to afford Compound 55 ((7R,8aS)-7-(2,3-dichloro-6-hydroxyphenyl)-4-oxo-hexahydropyrrolo[1,2-a]pyrazine-2-carboxamide) as an off-white solid (17.0 mg, 47*): LCMS (ESI) calc'd for C₁₄H₁₅Cl₂N₃O₃ [M+H]⁺: 344, 346 (3:2), found 344, 346 (3:2); ¹H NMR (400 MHz, CD₃OD) δ 7.27 (d, J=8.8 Hz, 1H), 6.77 (d, J=8.8 Hz, 1H), 4.48-4.27 (m, 3H), 4.21-4.13 (m, 1H), 3.96-3.87 (m, 1H), 3.82 (d, J=17.7 Hz, 1H), 3.59 (t, J=11.4, 9.7 Hz, 1H), 2.88 (dd, J=13.2, 10.5 Hz, 1H), 2.45-2.42 (m, 1H), 2.22-2.13 (m, 1H).

Example 38. Compounds 96-97 were prepared in an analogous fashion to that described for

Compound 55.

| Compound Number | Structure | Chemical Name | MS: (M + H)⁺ & ¹H MNR |
|---|---|---|---|
| 96 | 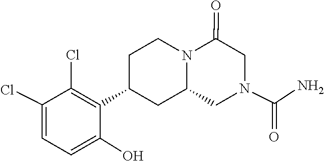 [SMILES: NC(=O)N1CC(=O)N2CC[C@@H](c3c(O)ccc(Cl)c3Cl)C[C@H]2C1] | (8R,9aS)-8-(2,3-dichloro-6- hydroxyphenyl)-4-oxo- hexahydro-1H-pyrido[1,2- a]pyrazine-2-carboxamide | [M + H]⁺: 358, 360 (3:2); ¹H NMR (400 MHz, CD₃OD) δ 7.21 (d, J = 8.8 Hz, 1H), 6.72 (d, J = 8.8 Hz, 1H), 4.88-4.71 (m, 1H), 4.19- 4.06 (m, 2H), 3.90-3.86 (m, 1H), 3.85-3.70 (m, 1H), 3.64-3.60 (m, 1H), 3.47-3.42 (m, 1H), 2.77 (dd, J = 12.9, 10.1 Hz, 1H), 2.49- 2.40 (m, 2H), 1.72-1.62 (m, 2H). |
| 97 | 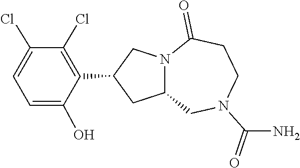 [SMILES: NC(=O)N1CCC(=O)N2C[C@@H](c3c(O)ccc(Cl)c3Cl)C[C@H]2C1] | (8R,9aS)-8-(2,3-dichloro-6- hydroxyphenyl)-5-oxo- hexahydro-1H-pyrrolo [1,2-a][1,4]diazepine-2- carboxamide | [M + H]⁺: 358, 360 (3:2); ¹H NMR (400 MHz, CD₃OD) δ 7.27 (d, J = 8.8 Hz, 1H), 6.76 (d, J =8.8 Hz, 1H), 4.27-4.22 (m, 1H), 4.19- 3.99 (m, 4H), 3.85-3.76 (m, 1H), 3.18-3.11 (m, 1H), 3.01-2.91 (m, 1H), 2.83-2.73 (m, 1H), 2.70- 2.57 (m, 2H), 2.35-2.24 (m, 1H). |

Example 39. Compound 30 ((7R,8aS)-7-(2,3-dichloro-6-hydroxyphenyl)-N,N-dimethyl-4-oxohexahydropyrrolo[1,2-a]pyrazine-2(1H)-carboxamide)

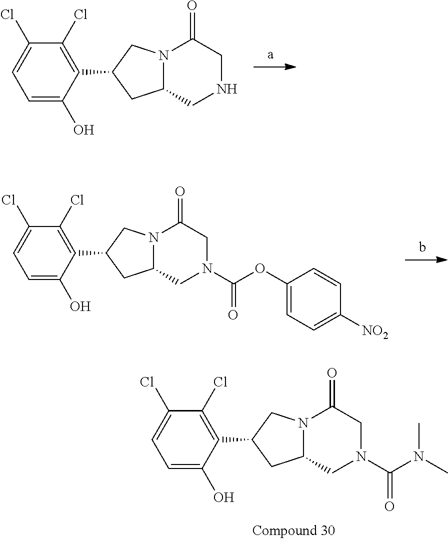 [SMILES: O=C1CNC[C@@H]2C[C@H](c3c(O)ccc(Cl)c3Cl)CN12 ; O=C(Oc1ccc([N+](=O)[O-])cc1)N1CC(=O)N2C[C@@H](c3c(O)ccc(Cl)c3Cl)C[C@H]2C1 ; CN(C)C(=O)N1CC(=O)N2C[C@@H](c3c(O)ccc(Cl)c3Cl)C[C@H]2C1]

Step a:

To a stirred solution of (7R,8aS)-7-(2,3-dichloro-6-hydroxyphenyl)-hexahydro-1H-pyrrolo[1,2-a]pyrazin-4-one hydrobromide (Intermediate 8, Example 7) (200 mg, 0.52 mmol) in DCM (2 mL) were added N,N-diisopropylethylamine (150 mg, 1.15 mmol) and 4-nitrophenyl chloroformate (84.0 mg, 0.42 mmol) at 0° C. The resulting solution was stirred for 1 h at 0° C. The reaction was diluted with water (20 mL) followed by extraction with EA (3×20 mL). The combined organic phases were washed with brine (2×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford 4-nitrophenyl (7R,8aS)-7-(2,3-dichloro-6-hydroxyphenyl)-4-oxo-hexahydropyrrolo[1,2-a]pyrazine-2-carboxylate as a light yellow solid (110 mg, 42%): LCMS (ESI) calc'd for C₂₀H₁₇Cl₂N₃O₆ [M+H]⁺: 466, 468 (3:2) found 466, 468 (3:2); ¹H NMR (400 MHz, DMSO-d₆) δ 10.42 (s, 1H), 8.34 (d, J=8.5 Hz, 2H), 7.50 (d, J=8.5 Hz, 2H), 7.36 (d, J=8.8 Hz, 1H), 6.85 (d, J=8.8 Hz, 1H), 4.57-4.09 (m, 3H), 4.09-3.81 (m, 3H), 3.49 (t, J=10.4 Hz, 1H), 3.05 (dt, J=72.4, 11.8 Hz, 1H), 2.31-2.08 (m, 2H).

Step b:

To a stirred solution of 4-nitrophenyl (7R,8aS)-7-(2,3-dichloro-6-hydroxyphenyl)-4-oxo-hexahydropyrrolo[1,2-a]pyrazine-2-carboxylate (30.0 mg, 0.06 mmol) and dimethylamine (9 mg, 0.19 mmol) in DMF (1 mL) was added K₂CO₃ (18 mg, 0.13 mmol) at room temperature. The resulting mixture was stirred for 1 h at 80° C. The reaction was filtered and the filtrate was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 25% to 50% in 8 min; Detector: UV 254/220 nm; Retention time: 5.85 min. The fractions containing the desired product were collected and concentrated under reduced pressure to afford Compound 30 ((7R,8aS)-7-(2,3-dichloro-6-hydroxyphenyl)-N,N-dimethyl-4-oxo-hexahydropyrrolo[1,2-a]pyrazine-2-carboxamide) as an off-white solid (14.0 mg, 55.53%): LCMS (ESI) calc'd for C₁₆H₁₉Cl₂N₃O₃ [M+H]⁺: 372, 374 (3:2) found 372, 374 (3:2); ¹H NMR (400 MHz, CD₃OD) δ 7.26 (d, J=8.8 Hz, 1H), 6.76 (d, J=8.8 Hz, 1H), 4.37-4.25 (m, 1H), 4.19 (dd, J=11.4, 9.2 Hz, 1H), 4.13-4.04 (m, 2H), 4.03-3.94 (m, 1H), 3.85 (d, J=17.6 Hz, 1H), 3.60-3.52 (m, 1H), 2.97-2.84 (m, 7H), 2.39-2.36 (m, 1H), 2.20-2.11 (m, 1H).

Example 40. Compounds 99-104 in an analogous fashion to that described for Compound 30.

| Compound | | | |
|---|---|---|---|
| Number | Structure | Chemical Name | MS: (M + H)⁺ & ¹H MNR |
| 99 | 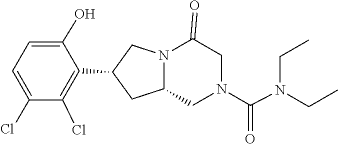 [SMILES: CCN(CC)C(=O)N1CC(=O)N2C[C@@H](c3c(O)ccc(Cl)c3Cl)C[C@H]2C1] | (7R,8aS)-7-(2,3- dichloro-6- hydroxyphenyl)- N,N-diethyl-4-oxo- hexahydropyrro[1,2-a]pyrazine-2- carboxamide | [M + H]⁺: 400, 402 (3:2); ¹H NMR (400 MHz, DMSO-d₆) δ 10.36 (s, 1H), 7.34 (d, J = 8.8 Hz, 1H),6.82 (d, J = 8.8 Hz, 1H), 4.17-4.03 (m, 1H), 4.03-3.93 (m, 1H), 3.93-3.65 (m, 4H), 3.24-3.08 (m, 5H), 2.78 (dd,J = 12.9, 10.1 Hz, 1H), 2.20-2.03 (m, 2H), 1.07 (t, J = 7.0 Hz, 6H). |
| 100 | 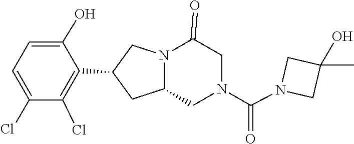 [SMILES: CC1(O)CN(C(=O)N2CC(=O)N3C[C@@H](c4c(O)ccc(Cl)c4Cl)C[C@H]3C2)C1] | (7R,8aS)-7-(2,3- dichloro-6- hydroxyphenyl)-2-(3- hydroxy-3- methylazetidine-1- carbonyl)- hexahydropyrro[1,2-a]pyrazine-4-one | [M + H]⁺:414, 416 (3:2); ¹H NMR (400 MHz, CD₃OD) δ 7.26 (d, J = 8.8 Hz, 1H),6.76 (d, J = 8.8 Hz, 1H), 4.37-4.12 (m, 4H), 4.00 (dd, J = 8.4, 2.4 Hz, 2H),3.94-3.81 (m, 4H), 3.57 (dd, J = 11.4, 9.6 Hz, 1H), 2.90 (dd, J = 13.2, 10.4 Hz, 1H), 2.42-2.38 (m, 1H), 2.21-2.15 (m, 1H), 1.49 (s, 3H). |
| 101 | 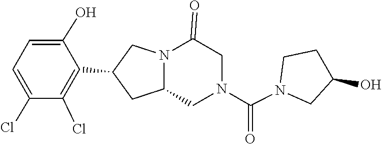 [SMILES: O=C(N1CC[C@@H](O)C1)N1CC(=O)N2C[C@@H](c3c(O)ccc(Cl)c3Cl)C[C@H]2C1] | (7R,8aS)-7-(2,3- dichloro-6- hydroxyphenyl)-2- [(3R)-3- hydroxypyrrolidine- 1-carbonyl]- hexahydropyrro[1,2-a]pyrazine-4-one | [M + H]⁺ 414, 416 (3:2); ¹H NMR CD₃OD) δ 7.26 (d, J = 8.8 Hz, 1H), 6.76 (d, J = 8.8 Hz, 1H), 4.46-4.40 (m, 1H), 4.37-4.17 (m, 3H), 4.11 (d, J = 17.6 Hz, 1H), 4.02 (tt, J = 10.6, 5.2 Hz, 1H), 3.91 (d, J = 17.5 Hz, 1H), 3.73 (td, J = 10.1, 7.2 Hz, 1H), 3.66 (dd, J = 11.4, 4.1Hz, 1H), 3.61-3.51 (m, 1H), |
| | | | 3.44-3.88 (m, 1H), 3.26 (dt, |
| | | | J = 11.4, 1.4 Hz, 1H), 2.85 (dd, J = |
| | | | 13.4, 10.3 Hz, 1H), 2.39-2.36 (m, 1H), |
| | | | 2.21-2.15 (m, 1H), 2.10-1.94 (m, 2H). |
| 102 | 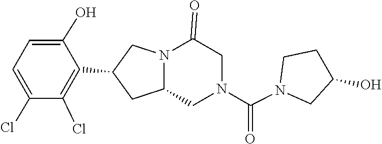 [SMILES: O=C(N1CC[C@H](O)C1)N1CC(=O)N2C[C@@H](c3c(O)ccc(Cl)c3Cl)C[C@H]2C1] | (7R,8aS)-7-(2,3- dichloro-6- hydroxyphenyl)-2- [(3S)-3- hydroxypyrrolidine- 1-carbonyl]- hexahydropyrro[1,2-a]pyrazine-4-one | [M + H]⁺ 414, 416 (3:2); ¹H NMR (400 MHz, CD₃OD) δ 7.26 (d,J = 8.8 Hz, 1H),6.77 (d, J = 8.8 Hz, 1H), 4.42-4.38 (m, 1H), 4.38-4.23 (m, 2H), 4.23-4.11 (m, 2H), 3.93 (tt, J = 10.4,4.7 Hz, 1H), 3.76 (d, J = 17.4 Hz, 1H), 3.65-3.49 (m, 4H), 3.37 (dt, J = 11.4, 1.4 Hz, 1H), 2.97 (dd, J = 13.3, 10.4 Hz, 1H), 2.40-2.38 |
| | | | (m, 1H), 2.21-2.15 (m, 1H), 2.07- |
| | | | 1.99 (m, 1H), 1.94-1.88 (m, 1H). |
| 103 | 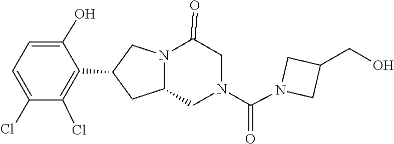 [SMILES: O=C(N1CC(CO)C1)N1CC(=O)N2C[C@@H](c3c(O)ccc(Cl)c3Cl)C[C@H]2C1] | (7R,8aS)-7-(2,3- dichloro-6- hydroxypheny1)-2- [3-(hydroxymethyl) azetidine-1- carbonyl]- hexahydropyrro[1,2-a]pyrazine-4-one | [M + H]⁺ 414, 416 (3:2); ¹H NMR (400 MHz, CD₃OD) δ 7.26 (d, J = 8.8 Hz, 1H),6.76 (d, J = 8.8 Hz, 1H), 4.38-4.25 (m, 2H), 4.25-4.13 (m, 3H), 4.08(t,J = 8.3 Hz, 1H), 3.95-3.76 (m, 4H), 3.70 (d, J= 6.3 Hz, 2H), 3.62- 3.52 (m, 1H), 2.89 (dd, J = 13.3, 10.4 Hz, 1H), 2.83-2.74 (m, 1H), 2.44-2.36 (m, 1H), 2.23-2.12 (m, 1H). |
| 104 | 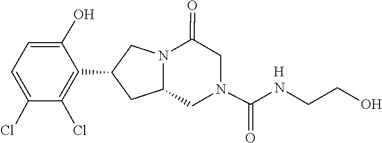 | (7R,8aS)-7-(2,3- dichloro-6- hydroxyphenyl)-N- (2-hydroxyethyl)-4-oxo- hexahydropyrro[1,2-a]pyrazine-2- carboxamide | [M + H]⁺ 388, 390 (3:2); ¹H NMR (400 MHz, CD₃OD) δ 7.27 (d, J = 8.8 Hz, 1H),6.77 (d, J = 8.8 Hz, 1H), 4.62-4.58 (m, 2H), 4.46 (dd, J = 13.2, 3.7 Hz, 1H),4.40-4.26 (m, 2H), 4.22- 4.13 (m, 1H), 3.94-3.77 (m, 2H), 3.66-3.54 (m, 3H), 2.86 (dd, J = 13.3, 10.4 Hz, 1H), 2.44-2.36 (m, 1H), 2.21-2.15 (m, 1H). |

### Example 41. Compound 105 ((7S,9aR)-7-(2,3-dichloro-6-hydroxyphenyl)-octahydropyrido[1,2-a]pyrazin-4-one isomer 1) and Compound 106 ((7S,9aS)-7-(2,3-dichloro-6-hydroxyphenyl)-octahydropyrido[1,2-a]pyrazin-4-one isomer 2)

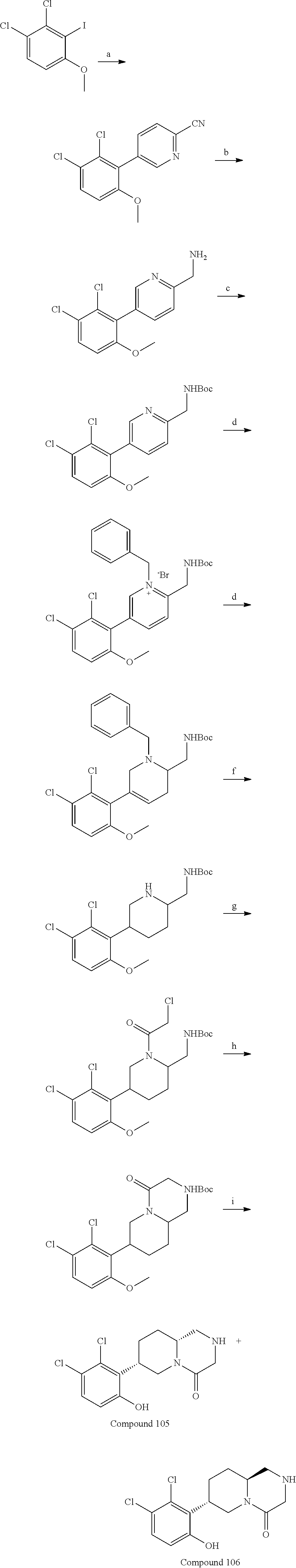

Step a:

To a solution of 1,2-dichloro-3-iodo-4-methoxybenzene (2.00 g, 6.60 mmol) and 5-(4,4,5,5-tetramethyl-1,3,2-dioxaborolan-2-yl)pyridine-2-carbonitrile (1.52 g, 6.60 mmol) in dioxane (20 mL) and H₂O (5 mL) were added Na₂CO₃ (2.10 g, 19.81 mmol) and Pd(dppf)Cl₂—CH₂Cl₂ (540 mg, 0.66 mmol) at room temperature under nitrogen atmosphere. The suspension was degassed under vacuum and purged with nitrogen atmosphere three times. Then the reaction was stirred at 80° C. for 3 h under nitrogen atmosphere. After cooling to room temperature, the mixture was diluted with water (50 mL). and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluting with PE/EA (3/1) to afford 5-(2,3-dichloro-6-methoxyphenyl)pyridine-2-carbonitrile as a yellow solid (1.50 g, 81%): LCMS (ESI) calc'd for C₁₃H₈Cl₂N₂O [M+H]⁺: 279, 281 (3:2) found 279, 281 (3:2); ¹H NMR (400 MHz, CDCl₃) δ 8.63 (dd, J=1.9, 1.1 Hz, 1H), 7.83-7.75 (m, 2H), 7.54 (d, J=9.0 Hz, 1H), 6.92 (d, J=9.0 Hz, 1H), 3.77 (s, 3H).

Step b:

To a stirred mixture of 5-(2,3-dichloro-6-methoxyphenyl)pyridine-2-carbonitrile (1.00 g, 3.58 mmol) in HCl (3.00 mL, 6 M) and MeOH (30 mL) was added PtO₂ (0.40 g, 1.76 mmol) at room temperature. The reaction mixture was degassed under vacuum and purged with hydrogen three times. The mixture was stirred at room temperature for 16 h under hydrogen atmosphere (1.5 atm). The reaction was filtered and concentrated under reduced pressure to afford 1-[5-(2,3-dichloro-6-methoxyphenyl)pyridin-2-yl]methanamine as a light yellow oil (1.50 g, crude), which was used directly in the next step without further purification: LCMS (ESI) calc'd for C₁₃H₁₂Cl₂N₂O [M+H]⁺: 283, 285 (3:2) found 283, 285 (3:2).

Step c:

To a solution of 1-[5-(2,3-dichloro-6-methoxyphenyl)pyridin-2-yl]methanamine (1.50 g, 5.30 mmol) in DCM (15 mL) and TEA (1.84 mL, 18.2 mmol) was added Boc₂O (1.16 g, 5.30 mmol) at room temperature. The reaction was stirred for 2 h, diluted with water (50 mL) and extracted with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluting with PE/EA (4/1) to afford tert-butyl N-[[5-(2,3-dichloro-6-methoxyphenyl)pyridin-2-yl]methyl]carbamate as a colorless oil (0.80 g, 58% overall two steps): LCMS (ESI) calc'd for C₁₈H₂OCl₂N₂O₃ [M+H]⁺: 383, 385 (3:2) found 383, 385 (3:2); ¹H NMR (400 MHz, CDCl₃) δ 8.45 (s, 1H), 7.61 (dd, J=8.0, 2.2 Hz, 1H), 7.48 (d, J=8.9 Hz, 1H), 7.38 (d, J=8.0 Hz, 1H), 6.89 (d, J=9.0 Hz, 1H), 5.65-5.60 (brs, 1H), 4.55 (d, J=5.2 Hz, 2H), 3.75 (s, 3H), 1.51 (s, 9H).

Step d:

To a solution of tert-butyl N-[[4-(2,3-dichloro-6-methoxyphenyl)pyridin-2-yl]methyl]carbamate (0.60 g, 1.57 mmol) in MeCN (15 mL) was added benzyl bromide (1.34 g, 7.85 mmol) at room temperature. The reaction was stirred at 80° C. for 12 h. Then the reaction mixture was concentrated under reduced pressure to afford 1-benzyl-2-[[(tert-butoxycarbonyl)amino]methyl]-4-(2,3-dichloro-6-methoxyphenyl)pyridin-1-ium bromide as a light brown oil (1.00 g, crude), which was used directly in the next step without purification: LCMS (ESI) calc'd for C₂₅H₂₇BrCl₂N₂O₃ [M]⁺: 473, 475 (3:2) found 473, 475 (3:2).

Step e:

To a solution of 1-benzyl-2-[[(tert-butoxycarbonyl)amino]methyl]-5-(2,3-dichloro-6-methoxyphenyl)pyridin-1-ium bromide (1.00 g, 1.81 mmol) in MeOH (10 mL) was added NaBH₄ (200 mg, 5.29 mmol) in portions at room temperature. The reaction was stirred at room temperature for 2 h. The resulting mixture was quenched with saturated aq. NH₄Cl (5 mL) followed by extraction with EA (3×50 mL). The combined organic layers were washed with brine (2×50 mL) and dried over anhydrous Na₂SO₄. After filtration, the filtrate was concentrated under reduced pressure. The residue was purified by silica gel column chromatography, eluting with PE/EA (5/1) to afford tert-butyl N-[[1-benzyl-5-(2,3-dichloro-6-methoxyphenyl)-3,6-dihydro-2H-pyridin-2-yl]methyl]carbamate as a light yellow oil (300 mg, 40% over two steps): LCMS (ESI) calc'd for C₂₅H₃₀Cl₂N₂O₃ [M+H]⁺: 477, 479 (3:2) found 477, 479 (3:2); ¹H NMR (400 MHz, CDCl₃) δ 7.45-7.38 (m, 2H), 7.37-7.26 (m, 4H), 6.71 (d, J=8.9 Hz, 1H), 5.70 (s, 1H), 5.25-5.20 (brs, 1H), 3.97-3.80 (m, 2H), 3.76 (s, 3H), 3.46-2.94 (m, 5H), 2.52 (d, J=18.1 Hz, 1H), 2.05 (d, J=14.4 Hz, 1H), 1.49 (s, 9H).

Step f:

To a solution of tert-butyl N-[[1-benzyl-5-(2,3-dichloro-6-methoxyphenyl)-3,6-dihydro-2H-pyridin-2-yl]methyl]carbamate (300 mg, 0.63 mmol) in AcOH (20 mL) was added PtO₂ (100 mg, 0.44 mmol) at room temperature. The reaction mixture was degassed under vacuum and purged with hydrogen three times. The mixture was stirred at room temperature for 16 h under hydrogen atmosphere (1.5 atm). Then the reaction was filtered and concentrated under reduced pressure. The residue was purified by silica gel column chromatography, eluting with PE/EA (1/1) to afford tert-butyl N-[[5-(2,3-dichloro-6-methoxyphenyl)piperidin-2-yl]methyl]carbamate as a light yellow solid (180 mg, 74%): LCMS (ESI) calc'd for C₁₈H₂₆Cl₂N₂O₃ [M+H]⁺: 389, 391 (3:2) found 389, 391 (3:2); ¹H NMR (400 MHz, CDCl₃) δ 7.31-7.27 (m, 1H), 6.74 (dd, J=8.9, 3.0 Hz, 1H), 3.88-3.78 (m, 3H), 3.59-3.42 (m, 2H), 3.37-3.30 (m, 1H), 3.16-2.88 (m, 2H), 2.88-2.60 (m, 1H), 2.29-2.06 (m, 2H), 1.87-1.50 (m, 2H), 1.47 (s, 9H).

Step g:

To a solution of tert-butyl N-[[5-(2,3-dichloro-6-methoxyphenyl)piperidin-2-yl]methyl]carbamate (180 mg, 0.46 mmol) and TEA (94.0 mg, 0.93 mmol) in DCM (3 mL) was added chloroacetyl chloride (57.0 mg, 0.51 mmol) at 0° C. The reaction was stirred at room temperature for 1 h, diluted with water (30 mL) and extracted with EA (3×30 mL). The combined organic layers were washed with brine (2×30 mL) and dried over anhydrous Na₂SO₄. After filtration, the filtrate was concentrated under reduced pressure to afford tert-butyl N-[[1-(2-chloroacetyl)-5-(2,3-dichloro-6-methoxyphenyl)piperidin-2-yl]methyl]carbamate as a light yellow oil (250 mg, crude), which was used directly in the next step without purification: LCMS (ESI) calc'd for C₂₀H₂₇C₁₃N₂O₄ [M+H]⁺: 465, 467 (1:1) found 465, 467 (1:1).

Step h:

To a solution of tert-butyl N-[[1-(2-chloroacetyl)-5-(2,3-dichloro-6-methoxyphenyl)piperidin-2-yl]methyl]carbamate (100 mg, 0.22 mmol) in DMF (2 mL) was added NaH (17.0 mg, 0.43 mmol, 60% in oil) at 0° C. under nitrogen atmosphere. The reaction was stirred at room temperature for 2 h. The reaction was quenched with saturated aq. NH₄Cl (5 mL), diluted with water (20 mL) and extracted with EA (2×20 mL). The combined organic phases were washed with brine (3×10 mL) and dried over anhydrous Na₂SO₄. After filtration, the filtrate was concentrated under reduced pressure. The residue was purified by silica gel column chromatography, eluting with PE/EA (3/1) to afford tert-butyl 7-(2,3-dichloro-6-methoxyphenyl)-4-oxo-hexahydro-1H-pyrido[1,2-a]pyrazine-2-carboxylate as a colorless oil (160 mg, 80% overall two steps): LCMS (ESI) calc'd for C₂₀H₂₆Cl₂N₂O₄ [M+H]⁺: 429, 431 (3:2) found 429, 431 (3:2).

Step i:

To a solution of tert-butyl 7-(2,3-dichloro-6-methoxyphenyl)-4-oxo-hexahydro-1H-pyrido[1,2-a]pyrazine-2-carboxylate (160 mg, 0.37 mmol) in DCM (2 mL) was added BBr₃ (0.32 mL, 1.28 mmol) dropwise at 0° C. The reaction mixture was stirred at room temperature for 3 h. The reaction mixture was quenched with MeOH (3 mL). The resulting mixture was concentrated under reduced pressure. The residue was purified by Pre-HPLC with the following conditions: Column: Column: Xselect CSH OBD Column, 30×150 mm, 5 μm; Mobile Phase A: Water (plus 0.05% TFA), Mobile Phase B: ACN; Flow rate: 60 mL/min; Gradient: 5% B to 30% B in 9 min; Detector: UV 220 nm; Retention Time 1: 9.07 min, Retention Time 2: 9.42 min. The faster-eluting enantiomer at 9.07 min was obtained assumed Compound 105 ((7S,9aR)-rel-7-(2,3-dichloro-6-hydroxyphenyl)-octahydropyrido[1,2-a]pyrazin-4-one) as an off-white foam (30.0 mg, 26%): LCMS (ESI) calc'd for C₁₄H₁₆Cl₂N₂O₂ [M+H]⁺: 315, 317 (3:2) found 315, 317 (3:2). The slower-eluting enantiomer at 9.42 min was obtained assumed Compound 106 ((7S,9aS)-rel-7-(2,3-dichloro-6-hydroxyphenyl)-octahydropyrido[1,2-a]pyrazin-4-one) as an off-white foam (30.0 mg, 26%): LCMS (ESI) calc'd for C₁₄H₁₆Cl₂N₂O₂ [M+H]⁺: 315, 317 (3:2) found 315, 317 (3:2).

### Example 42. Compound 26 ((7S,9aR)-rel-7-(2,3-dichloro-6-hydroxyphenyl)-2-(2-hydroxyacetyl)-hexahydro-1H-pyrido[1,2-a]pyrazin-4-one)

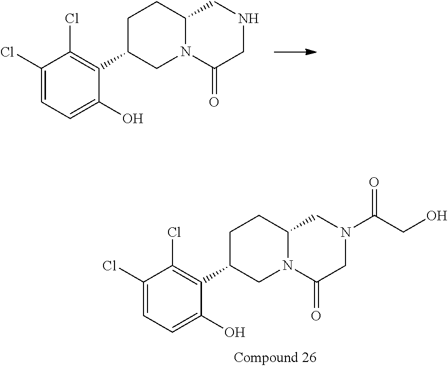 [SMILES: O=C1CNC[C@H]2CC[C@@H](c3c(O)ccc(Cl)c3Cl)CN12 ; O=C(CO)N1CC(=O)N2C[C@H](c3c(O)ccc(Cl)c3Cl)CC[C@@H]2C1]

To a stirred solution of glycolic acid (15.0 mg, 0.19 mmol), EDCI (46.0 mg, 0.24 mmol) and HOBT (32.0 mg, 0.24 mmol) in DMF (2 mL) were added (7S,9aR)-rel-7-(2,3-dichloro-6-hydroxyphenyl)-octahydropyrido[1,2-a]pyrazin-4-one (30.0 mg, 0.10 mmol) and TEA (39.0 mg, 0.38 mmol) at room temperature. The reaction was stirred at room temperature for 1 h, diluted with water (20 mL) and extracted with EA (3×20 mL). The combined organic phases were washed with brine (3×20 mL) and dried over anhydrous Na₂SO₄. After filtration, the filtrate was concentrated under reduced pressure. The residue was purified with Prep-HPLC with the following conditions: Column: Xselect CSH OBD Column, 30×150 mm, 5 μm; Mobile Phase A: Water (plus 0.05% TFA), Mobile Phase B: ACN; Flow rate: 60 mL/min; Gradient: 20% B to 43% B in 7 min; Detector: UV 220 nm; Retention time: 6.68 min. The fractions containing the desired product were collected and concentrated under reduced pressure to afford Compound 26 ((7S,9aR)-rel-7-(2,3-dichloro-6-hydroxyphenyl)-2-(2-hydroxyacetyl)-hexahydro-1H-pyrido[1,2-a]pyrazin-4-one) as an off-white solid (10.2 mg, 29%): LCMS (ESI) calc'd for C₁₆H₁₈Cl₂N₂O₄ [M+H]⁺: 373, 375 (3:2), found 373, 375 (3:2); ¹H NMR (400 MHz, CD₃OD) δ 7.24 (d, J=8.7 Hz, 1H), 6.76 (d, J=8.8 Hz, 1H), 4.50-4.20 (m, 4H), 4.20-4.07 (m, 1H), 4.07-3.82 (m, 3H), 3.74-3.59 (m, 1H), 3.31-3.08 (m, 1H), 2.36-2.22 (m, 1H), 1.98-1.81 (m, 3H).

### Example 43. Compound 13 ((7S,9aS)-rel-7-(2,3-dichloro-6-hydroxyphenyl)-2-(2-hydroxyacetyl)-hexahydro-1H-pyrido[1,2-a]pyrazin-4-one)

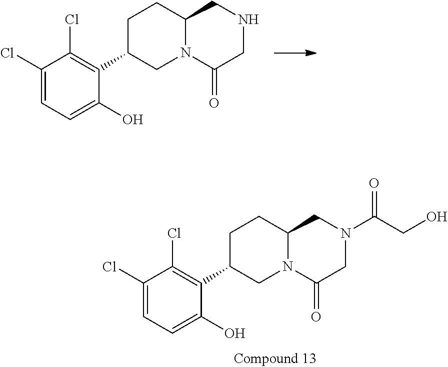 [SMILES: O=C1CNC[C@@H]2CC[C@@H](c3c(O)ccc(Cl)c3Cl)CN12 ; O=C(CO)N1CC(=O)N2C[C@H](c3c(O)ccc(Cl)c3Cl)CC[C@H]2C1]

To a stirred solution of glycolic acid (15.0 mg, 0.19 mmol), EDCI (46.0 mg, 0.24 mmol) and HOBT (32.0 mg, 0.24 mmol) in DMF (2 mL) were added (7S,9aS)-rel-7-(2,3-dichloro-6-hydroxyphenyl)-octahydropyrido[1,2-a]pyrazin-4-one (30.0 mg, 0.10 mmol) and TEA (39.0 mg, 0.38 mmol) at room temperature. The reaction was stirred at room temperature for 1 h, diluted with water (20 mL) and extracted with EA (3×10 mL). The combined organic phases were washed with brine (3×20 mL) and dried over anhydrous Na₂SO₄. After filtration, the filtrate was concentrated under reduced pressure. The residue was purified with Prep-HPLC with the following conditions: Column: Xselect CSH OBD Column, 30×150 mm, 5 μm; Mobile Phase A: Water (plus 0.05% TFA), Mobile Phase B: ACN; Flow rate: 60 mL/min; Gradient: 20% B to 40% B in 7 min; Detector: UV 220 nm; Retention time: 6.30 min. The fractions containing the desired product were collected and concentrated under reduced pressure to afford Compound 13 ((7S,9aS)-rel-7-(2,3-dichloro-6-hydroxyphenyl)-2-(2-hydroxyacetyl)-hexahydro-1H-pyrido[1,2-a]pyrazin-4-one) as an off-white solid (10.2 mg, 29%): LCMS (ESI) calc'd for C₁₆H₁₈Cl₂N₂O₄ [M+H]⁺: 373, 375 (3:2) found 373, 375 (3:2); ¹H NMR (400 MHz, CD₃OD) δ 7.23 (d, J=8.8 Hz, 1H), 6.74 (d, J=8.8 Hz, 1H), 4.51-4.40 (m, 1H), 4.38-4.06 (m, 4H), 4.06-3.88 (m, 1H), 3.74-3.39 (m, 4H), 2.66-2.52 (m, 1H), 2.01-1.74 (m, 2H), 1.64-1.47 (m, 1H).

Example 44. Compounds 109-122 were prepared in an analogous fashion to an example disclosed herein and/or analogous to known methods in the art.

| Compound | | | |
|---|---|---|---|
| Number | Structure | Chemical Name | MS: (M + H)⁺ & ¹H MNR |
| 109 | 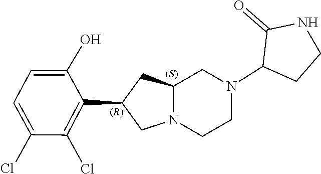 | 3-[(7R,8aS)-7- (2,3-dichloro-6- hydroxyphenyl)- 4-oxo- hexahydropyrrolo [1,2-a]pyrazin-2- yl]pyrrolidin-2- one | [M + H]⁺: 384, 386 (3:2); ¹H NMR (400 MHz, CD₃OD) δ 7.25 (d, J = 8.8 Hz, 1H), 6.75 (d, J = 8.8 Hz, 1H), 4.38-4.26 (m, 1H), 4.16 (dd, J = 11.6, 8.9 Hz, 1H), 3.93 (td, J = 10.5, 5.1 Hz, 1H), 3.72-3.61 (m, 2H), 3.58-3.49 (m, 2H), 3.41-3.35 (m, 2H), 3.24-3.15 (m, 1H), 2.69 (dd, J = 11.5, 10.0 Hz, 0.5H), 2.48 (dd, J = 11.5, 10.1 Hz, 0.5H), 2.41-2.24 (m, 2H), 2.24-2.05 (m, 2H). |
| 110 | 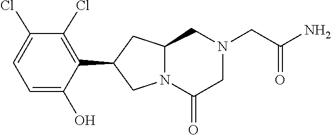 [SMILES: NC(=O)CN1CC(=O)N2C[C@@H](c3c(O)ccc(Cl)c3Cl)C[C@H]2C1] | 2-[(7R,8aS)-7- (2,3-dichloro-6- hydroxyphenyl)- 4-oxo- hexahydropyrrolo [1,2-a]pyrazin-2- yl]acetamide | [M + H]⁺ 358, 360 (3:2); ¹H NMR (400 MHz, CD₃OD) δ 7.26 (d, J = 8.7 Hz, 1H), 6.76 (d, J = 8.8 Hz, 1H), 4.48-4.32 (m, 1H), 4.24-4.08 (m, 2H), 3.95-3.73 (m, 4H), 3.71-3.58 (m, 2H), 3.05-2.92 (m, 1H), 2.47-2.33 (m, 1H), 2.28-2.17 (m, 1H). |
| 111 | 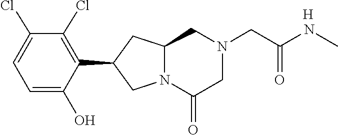 | 2-[(7R,8aS)-7- (2,3-dichloro-6- hydroxyphenyl)- 4-oxo- hexahydropyrrolo [1,2-a]pyrazin-2- yl]-N- methylacetamide | [M + H]⁺: 372, 374 (3:2); ¹H NMR (400 MHz, CD₃OD) δ 7.25 (d, J = 8.7 Hz, 1H), 6.76 (d, J =8.8 Hz, 1H), 4.37-4.27 (m, 1H), 4.21-(m, 2H), 3.29-3.20 (m, 3H), 4.11 (m, 1H), 4.09-3.98 (m, 1H), 3.62-3.49 3.07 (d, J = 16.6 Hz, 1H), 2.80 (s, 3H), 2.47-2.41 (m, 1H), 2.38-2.29 (m, 1H), 2.16-2.09 (m, 1H). |
| 112 | 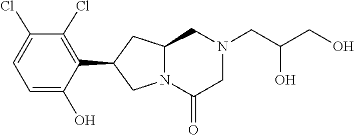 [SMILES: O=C1CN(CC(O)CO)C[C@@H]2C[C@H](c3c(O)ccc(Cl)c3Cl)CN12] | (7R,8aS)-7-(2,3- dichloro-6- hydroxyphenyl)- 2-(2,3- dihydroxypropyl)- hexahydropyrrolo [l,2-a]pyrazin-4- one | [M + H]⁺: 375, 377 (3:2); ¹H NMR (400 MHz, CD₃OD) δ 7.27 (d, J = 8.8 Hz, 1H), 6.77 (d, J =8.7 Hz, 1H), 4.47- 4.32 (m, 1H), 4.29-4.05 (m, 5H), 3.96 (dd, J = 17.5, 6.7 Hz, 1H), 3.69-3.53 (m, 3H), 3.45-3.20 (m, 3H), 2.55-2.40 (m, 1H), 2.35-2.21 (m, 1 H). |
| 113 | 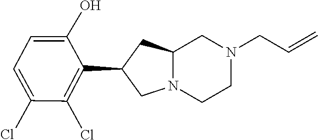 | (7R,8aS)-7-(2,3- dichloro-6- hydroxyphenyl)- 2-(prop-2-en-1- yl)- hexahydropyrrolo [l,2-a]pyrazin-4- one | [M + H]⁺ 341, 343 (3:2); ¹H NMR (400 MHz, CD₃OD) δ 7.29 (d, J = 8.8 Hz, 1H), 6.79 (d, J = 8.8 Hz, 1H), 6.09-5.94 (m, 1H), 5.70-5.61 (m, 2H),4.46-4.32 (m, 1H), 4.22 (dd, J = 11.5, 9.1Hz, 1H), 4.14-4.03 (m, 1H), 4.00-3.80 (m, 4H), 3.77- 3.70 (m, 1H), 3.65-3.60 (m, 1H), 3.13-3.03 (m, 1H),2.51-2.42 (m, 1H), 2.30-2.23 (m, 1H). |
| 114 | 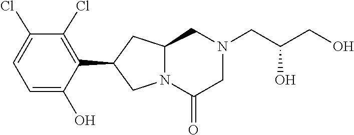 [SMILES: O=C1CN(C[C@@H](O)CO)C[C@@H]2C[C@H](c3c(O)ccc(Cl)c3Cl)CN12] | (7R,8aS)-7-(2,3- dichloro-6- hydroxyphenyl)- 2-[2,3- dihydroxypropyl]- hexahydropyrrolo [1,2-a]pyrazin-4- one isomer l | [M + H]⁺: 375, 377 (3:2); ¹H NMR (400 MHz, CD₃OD) δ 7.29 (d, J = 8.8 Hz, 1H), 6.79 (d, J = 8.7 Hz, 1H), 4.47-4.32 (m, 1H), 4.29-3.99 (m, 5H), 3.87 (dd, J = 17.5, 6.7 Hz, 1H), 3.69-3.54 (m, 3H), 3.45-3.20 (m, 3H), 2.51-2.42 (m, 1H), 2.30-2.24 (m, 1 H); |
| 115 | 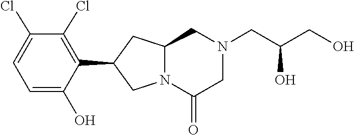 [SMILES: O=C1CN(C[C@H](O)CO)C[C@@H]2C[C@H](c3c(O)ccc(Cl)c3Cl)CN12] | (7R,8aS)-7-(2,3- dichloro-6- hydroxyphenyl)- 2-[2,3- dihydroxypropyl]- hexahydropyrrolo [1,2-a]pyrazin-4- one isomer 2 | [M + H]⁺: 375, 377 (3:2); ¹H NMR (400 MHz, CD₃OD) δ 7.29 (d, J = 8.8 Hz, 1H), 6.79 (d, J = 8.7 Hz, 1H), 4.47-4.32 (m, 1H), 4.25-4.05 (m, 5H), 3.92 (dd, J = 17.5, 6.7 Hz, 1H), 3.66-3.57 (m, 3H), 3.45-3.20 (m, 3H), 2.52-2.43 (m, 1H), 2.33-2.26 (m, 1H). |
| 116 | 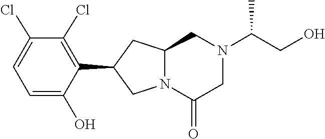 [SMILES: C[C@H](CO)N1CC(=O)N2C[C@@H](c3c(O)ccc(Cl)c3Cl)C[C@H]2C1] | (7R,8aS)-7-(2,3- dichloro-6-hydroxyphenyl)-2-[1-hydroxypropan- 2-yl]- hexahydropyrrolo [1,2-a]pyrazin-4- one isomer l | [M + H]⁺: 359, 361 (3:2); ¹H NMR (400 MHz, CD₃OD) δ 7.25 (d, J = 8.8 Hz, 1H), 6.75 (d, J = 8.8 Hz, 1H), 4.36-4.26 (m, 1H), 4.15(dd, J = 11.6, 8.9 Hz, 1H), 3.96- 3.88 (m, 1H), 3.64 (dd, J = 11.4, 6.9 Hz, 1H), 3.58-3.42 (m, 3H), 3.33-3.22 (m, 2H), 2.94-2.87 (m, 1H), 2.47 (dd, J = 11.6, 9.9 Hz, 1H), 2.39-2.30 (m, 1H), 2.15-2.07 (m, 1H), 1.08 (d, J = 6.7 Hz, 3H). |
| 117 | 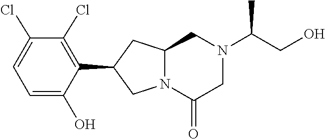 [SMILES: C[C@@H](CO)N1CC(=O)N2C[C@@H](c3c(O)ccc(Cl)c3Cl)C[C@H]2C1] | (7R,8aS)-7-(2,3- dichloro-6-hydroxyphenyl)-2-[1-hydroxypropan- 2-yl]- hexahydropyrrolo [1,2-a]pyrazin-4- one isomer 2 | [M + H]⁺: 359, 361 (3:2); ¹H NMR (400 MHz, CD₃OD) δ 7.25 (d, J = 8.8 Hz, 1H), 6.75 (d, J = 8.8 Hz, 1H), 4.36-4.25 (m, 1H), 4.15(dd,J = 11.6, 8.9 Hz, 1H), 3.94- 3.85 (m, 1H), 3.62 (dd, J = 11.4, 7.3 Hz, 1H), 3.58-3.42 (m, 3H), 3.33-3.22 (m, 2H), 2.87-2.79 (m, 1H), 2.40-2.27 (m, 2H), 2.15-2.07 (m, 1H), 1.07 (d, J = 6.6 Hz, 3H). |
| 118 | 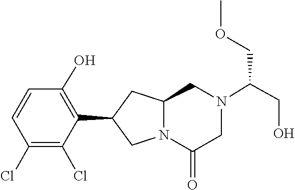 [SMILES: COC[C@H](CO)N1CC(=O)N2C[C@@H](c3c(O)ccc(Cl)c3Cl)C[C@H]2C1] | (7R,8aS)-7-(2,3- hydroxyphenyl)- 2-[1-hydroxy-3- methoxypropan- 2-yl]- hexahydropyrrolo [1,2-a]pyrazin-4- one isomer l | [M + H]⁺: 389, 391 (3:2); ¹H NMR (400 MHz, CD₃OD) δ 7.24 (d, J = 8.8 Hz, 1H), 6.75 (d, J = 8.8 Hz, 1H), 4.37-4.23 (m, 1H), 4.15(dd,J = 11.5, 8.9 Hz, 1H), 3.92-3.84 (m, 1H), 3.75-3.59 (m, 3H), 3.56-3.39 (m, 5H), 3.37 (s, 3H), 2.96-2.85 (m, 1H), 2.63 (dd, J = 11.8, 10.0 Hz, 1H), 2.38-2.28 (m, 1H), 2.13-2.05 (m, 1H). |
| 119 | 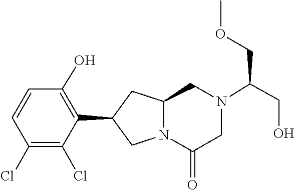 [SMILES: COC[C@@H](CO)N1CC(=O)N2C[C@@H](c3c(O)ccc(Cl)c3Cl)C[C@H]2C1] | (7R,8aS)-7-(2,3- dichloro-6- hydroxyphenyl)- 2-[1-hydroxy-3- methoxypropan- 2-yl]- hexahydropyrrolo [1,2-a]pyrazin-4- one isomer 2 | [M + H]⁺: 389, 391 (3:2); ¹H NMR (400 MHz, CD₃OD) δ 7.24 (d, J = 8.8 Hz, 1H), 6.75 (d, J = 8.7 Hz, 1H), 4.37-4.23 (m, 1H), 4.15 (dd,J = 11.5, 8.9 Hz, 1H), 3.92-3.84 (m, 1H), 3.74-3.45 (m, 7H), 3.41 (dd, J = 11.7, 4.0 Hz, 1H), 3.36(s, 3H), 2.96-2.85 (m, 1H), 2.51 (dd, J = 11.7, 9.9 Hz, 1H), 2.38-2.28 (m, 1H), 2.13-2.05 (m, 1H). |
| 120 | 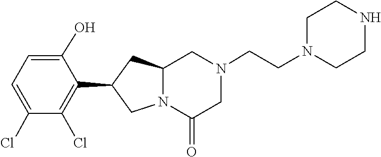 [SMILES: O=C1CN(CCN2CCNCC2)C[C@@H]2C[C@H](c3c(O)ccc(Cl)c3Cl)CN12] | (7R,8aS)-7-(2,3- dichloro-6- hydroxyphenyl)- 2-[2-(piperazin-1- yl)ethyl]- hexahydropyrrolo [1,2-a]pyrazin-4- one | [M + H]⁺: 413, 415 (3:2); ¹H NMR (400 MHz, CD₃OD) δ 7.24 (d, J = 8.7 Hz, 1H), 6.75 (d, J = 8.8 Hz, 1H), 4.37-4.26 (m, 1H), 4.16 (dd, J = 11.5, 8.8 Hz, 1H), 3.98-3.88 (m, 1H), 3.62-3.46 (m, 2H), 3.40-3.33 (m, 1H), 2.96-2.86 (m, 5H), 2.78-2.58 (m,8H), 2.39-2.19 (m, 2H), 2.16-2.09 (m, 1H). |
| 121 | 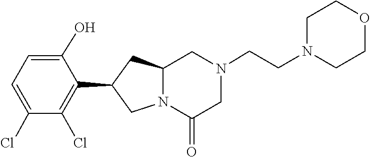 [SMILES: O=C1CN(CCN2CCOCC2)C[C@@H]2C[C@H](c3c(O)ccc(Cl)c3Cl)CN12] | (7R,8aS)-7-(2,3- dichloro-6- hydroxyphenyl)- 2-[2-(morpholin- 4-yl)ethyl]- hexahydropyrrolo [1,2-a]pyrazin-4- one | [M + H]⁺:414, 416 (3:2); ¹H NMR (400 MHz, CD₃OD) δ 8.25 (s, 1H), 7.26 (d, J = 8.8 Hz, 1H),6.76 (d, J = 8.8 Hz, 1H), 4.37-4.26 (m, 1H), 4.22-4.12 (m, 1H), 4.01-3.92 (m, 1H), 3.90-3.80 (m, 4H), 3.61 (d, J = 16.7 Hz, 1H), 3.57-3.47 (m, 1H), 3.38 (dd, J 11.6, 3.8 Hz, 1H), 3.08-2.74 (m, 9H),2.41-2.27 (m, 2H), 2.16-2.10 (m, 1H). |
| 122 | 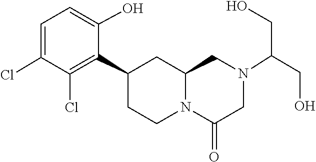 [SMILES: O=C1CN(C(CO)CO)C[C@@H]2C[C@H](c3c(O)ccc(Cl)c3Cl)CCN12] | (8R,9aS)-8-(2,3- dichloro-6- hydroxyphenyl)- 2-(1,3- dihydroxypropan- 2-yl)-hexahydro- 1H-pyrido[1,2-a] pyrazin-4-one | [M + H]⁺: 389, 391 (3:2); ¹H NMR (400 MHz, CD₃OD) δ 7.21 (d, J = 8.7 Hz, 1H), 6.72 (d, J = 8.8 Hz, 1H), 4.79-4.71 (m, 1H), 3.93-3.61 (m, 8H), 3.59-3.49 (m, 1H), 3.12-3.00 (m, 2H), 2.74 (td, J = 13.2, 3.0 Hz, 1H), 2.56-2.39 (m, 2H), 1.70 (dd, J = 19.9, 13.6 Hz, 2H). |

### Example 45. Compound 123 ((7R,8aS)-7-(2,3-dichloro-6-hydroxyphenyl)-2-(3-hydroxycyclobutyl)-hexahydropyrrolo[1,2-a]pyrazin-4-one)

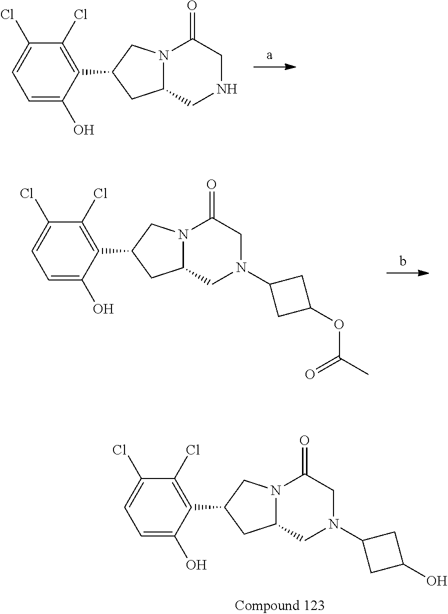 [SMILES: O=C1CNC[C@@H]2C[C@H](c3c(O)ccc(Cl)c3Cl)CN12 ; CC(=O)OC1CC(N2CC(=O)N3C[C@@H](c4c(O)ccc(Cl)c4Cl)C[C@H]3C2)C1 ; O=C1CN(C2CC(O)C2)C[C@@H]2C[C@H](c3c(O)ccc(Cl)c3Cl)CN12]

Step a:

To a stirred mixture of (7R,8aS)-7-(2,3-dichloro-6-hydroxyphenyl)-hexahydro-1H-pyrrolo[1,2-a]pyrazin-4-one HBr salt (Intermediate 8, Example 7) (40.0 mg, 0.13 mmol), NaOAc (43.0 mg, 0.53 mmol) and 3-oxocyclobutyl acetate (51 mg, 0.40 mmol) in DCM (4 mL) was added NaBH(OAc)₃ (0.113 g, 0.53 mmol) at room temperature. The reaction was for 4 h, quenched with saturated aq. NH₄Cl (30 mL) and extracted with EA (3×20 mL). The combined organic layers were washed with brine (3×30 mL) and dried over anhydrous Na₂SO₄. After filtration, the filtrate was concentrated under reduced pressure to afford 3-[(7aR,8aS)-7-(2,3-dichloro-6-hydroxyphenyl)-4-oxo-hexahydropyrrolo[1,2-a]pyrazin-2-yl]cyclobutyl acetate as an off-white solid (0.100 g, crude), which was used in the next step directly without purification: LCMS (ESI) calc'd for C₁₉H₂₂Cl₂N₂O₄ [M+H]⁺: 413, 415 (3:2) found 413, 415 (3:2).

Step b:

A mixture of 3-[(7R,8aS)-7-(2,3-dichloro-6-hydroxyphenyl)-4-oxo-hexahydropyrrolo[1,2-a]pyrazin-2-yl]cyclobutyl acetate (80.0 mg, 0.19 mmol) and K₂CO₃ (80.0 mg, 0.58 mmol) in MeOH (2 mL) was stirred at room temperature for 2 h. The reaction was concentrated under reduced pressure. The residue was purified by Prep-HPLC with the following conditions: Column: XBridge Shield RP18 OBD Column, 30×150 mm, 5 μm; Mobile Phase A: water (plus 0.05% TFA), Mobile Phase B: ACN; Flow rate: 60 mL/min; Gradient: 5% B to 35% B in 7 min; Detector: UV 220 nm; Retention time: 6.77 min. The fractions containing desired product were collected and concentrated under reduced pressure to afford Compound 123 ((7R,8aS)-7-(2,3-dichloro-6-hydroxyphenyl)-2-(3-hydroxycyclobutyl)-hexahydropyrrolo[1,2-a]pyrazin-4-one) as an off-white solid (23.4 mg, 33%). LCMS (ESI) calc'd for C₁₇H₂₀Cl₂N₂O₃ [M+H]⁺: 371, 373 (3:2) found 371, 373 (3:2); ¹H NMR (400 MHz, CD₃OD) δ 7.29 (d, J=8.8 Hz, 1H), 6.78 (d, J=8.8 Hz, 1H), 4.54-4.34 (m, 1H), 4.27-4.15 (m, 1H), 4.16-3.98 (m, 3H), 3.93 (d, J=11.5 Hz, 1H), 3.77-3.54 (m, 2H), 3.45-3.36 (m, 1H), 3.13-2.96 (m, 1H), 2.87-2.54 (m, 2H), 2.49-2.43 (m, 1H), 2.38-2.06 (m, 3H).

Example 46. Compounds 124-147 were prepared in an analogous fashion as that described for Compound 123.

| Compound | | | |
|---|---|---|---|
| Number | Structure | Chemical Name | MS: (M + H)⁺ & ¹H MNR |
| 124 | 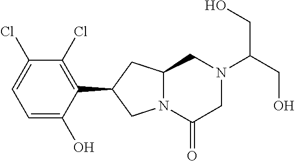 [SMILES: O=C1CN(C(CO)CO)C[C@@H]2C[C@H](c3c(O)ccc(Cl)c3Cl)CN12] | (7R,8aS)-7-(2,3- dichloro-6- hydroxyphenyl)-2- (1,3- dihydroxypropan- 2-yl)- hexahydropyrrolo [1,2-a]pyrazin-4- one | [M + H]⁺: 375, 377 (3:2); ¹H NMR (400 MHz, CD₃OD) δ 7.25 (d, J = 8.7 Hz, 1H), 6.75 (d, J = 8.8 Hz, 1H), 4.37-4.23 (m, 1H), 4.15 (dd, J = 11.6, 8.9 Hz, 1H), 3.95-3.86 (m, 1H), 3.79-3.62 (m, 4H), 3.55- 3.48 (m, 3H), 3.41 (dd, J = 11.7, 4.0 Hz, 1H), 2.86-2.76 (m, 1H), 2.62 (dd, J = 11.7, |
| | | | 10.0 Hz, 1H), 2.39-2.31 (m, |
| | | | 1H), 2.13-2.05 (m, 1H). |
| 125 | 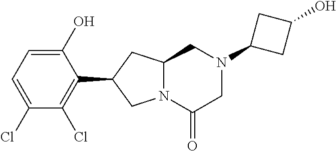 [SMILES: O=C1CN([C@H]2C[C@H](O)C2)C[C@@H]2C[C@H](c3c(O)ccc(Cl)c3Cl)CN12] | (7R,8aS)-7-(2,3- dichloro-6- hydroxyphenyl)-2- [(1r,3S)-rel-3- hydroxycyclobutyl] hexahydropyrrolo [1,2-a]pyrazin-4- one | [M + H]⁺: 371, 373 (3:2); ¹H NMR (400 MHz, CD₃OD) δ 7.25 (d, J = 8.8 Hz, 1H), 6.75 (d, J = 8.8 Hz, 1H), 5.24-5.15 (m, 1H), 4.42-4.26 (m, 2H), 4.21-4.11 (m, 1H), 3.96-3.82 (m, 2H), 3.61-3.51 (m, 2H), 3.14-3.08 (m, 1H), 2.70 (d, J = 16.8 Hz, 1H), 2.37-2.25 (m, 2H), 2.18-2.04 (m, 3H), 2.03- |
| | | | 1.95 (m, 1H). |
| 126 | 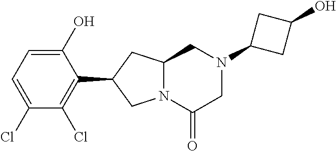 [SMILES: O=C1CN([C@H]2C[C@@H](O)C2)C[C@@H]2C[C@H](c3c(O)ccc(Cl)c3Cl)CN12] | (7R,8aS)-7-(2,3- dichloro-6- hydroxyphenyl)-2- [(1s,3R)-rel-3- hydroxycyclobutyl] hexahydropyrrolo [1,2-a]pyrazin-4- one | [M + H]⁺: 371, 373 (3:2); ¹H NMR (400 MHz, CD₃OD) δ 7.25 (d, J = 8.8 Hz, 1H), 6.75 (d, J = 8.8 Hz, 1H), 4.39-4.24 (m, 1H), 4.15 (dd, J = 11.6, 8.8 Hz, 1H), 4.05-3.80 (m, 2H), 3.60-3.51 (m, 1H), 3.44 (d, J = 16.8 Hz, 1H), 3.31- 3.25 (m, 1H), 2.74 (d, J = 16.7 Hz, 1H), 2.61-2.45 (m, |
| | | | 3H), 2.34-2.30 (m, 1H), 2.17- |
| | | | 2.09 (m, 1H), 2.09-2.00 (m, |
| | | | 1H), 1.86-1.75 (m, 2H). |
| 127 | 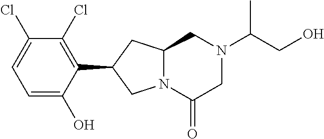 [SMILES: CC(CO)N1CC(=O)N2C[C@@H](c3c(O)ccc(Cl)c3Cl)C[C@H]2C1] | (7R,8aS)-7-(2,3- dichloro-6- hydroxyphenyl)-2- (1-hydroxypropan- 2-yl)- hexahydropyrrolo [1,2-a]pyrazin-4- one | [M + H]⁺: 359, 361 (3:2); ¹H NMR (400 MHz, CD₃OD) δ 7.29 (d, J = 8.8 Hz, 1H), 6.79 (d, J = 8.8 Hz, 1H), 4.44-4.34 (m, 1H), 4.25-4.20 (m, 1H), 4.17-4.04 (m, 1H), 4.04-3.94 (m, 2H), 3.96-3.85 (m, 1H), 3.76-3.70 (m, 1H), 3.67-3.59 (m, 2H), 3.31-3.15 (m, 1H), |
| | | | 3.03-2.99 (m, 1H), 2.55-2.44 |
| | | | (m, 1H), 2.33-2.24 (m, 1H), |
| | | | 1.42-1.37 (m, 3H). |
| 128 | 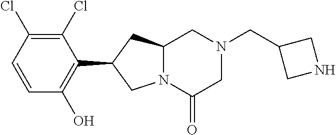 [SMILES: O=C1CN(CC2CNC2)C[C@@H]2C[C@H](c3c(O)ccc(Cl)c3Cl)CN12] | (7R,8aS)-2- (azetidin-3- ylmethyl)-7-(2,3- dichloro-6- hydroxyphenyl)- hexahydropyrrolo [1,2-a]pyrazin-4- one | [M + H]⁺: 370, 372 (3:2); ¹H NMR (400 MHz, CD₃OD) δ 7.25 (d, J = 8.8 Hz, 1H), 6.77 (d, J = 8.8 Hz, 1H), 4.40-4.27 (m, 1H), 4.26-4.14 (m, 3H), 4.09-3.94 (m, 3H), 3.81 (d, J = 16.4 Hz, 1H), 3.64 (dd, J = 11.8, 3.7 Hz, 1H), 3.60-3.54 (m, 1H), 3.45-3.37 (m, 2H), |
| | | | 3.30-3.20 (m, 2H), 2.82-2.76 |
| | | | (m, 1H), 2.44-2.34 (m, 1H), |
| | | | 2.24-2.16 (m, 1H). |
| 129 | 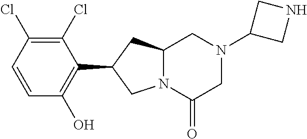 [SMILES: O=C1CN(C2CNC2)C[C@@H]2C[C@H](c3c(O)ccc(Cl)c3Cl)CN12] | (7R,8aS)-2- (azetidin-3-yl)-7- (2,3-dichloro-6- hydroxyphenyl)- hexahydropyrrolo [1,2-a]pyrazin-4-one | [M + H]⁺: 356, 358 (3:2); ¹H NMR (400 MHz, DMSO-d) δ 10.52-10.41 (brs, 1H), 8.87- 8.71 (brs, 2H), 7.34 (d, J = 8.8 Hz, 1H), 6.84 (d, J = 8.8 Hz, 1H), 4.18-4.07 (m, 1H), 4.05- 3.85 (m, 6H), 3.56-3.49 (m, 1H), 3.45-3.35 (m, 2H), 3.25- 3.18 (m, 1H), 2.75 (d, J = |
| | | | 16.2 Hz, 1H), 2.19-1.98 (m, |
| | | | 3H). |
| 130 | 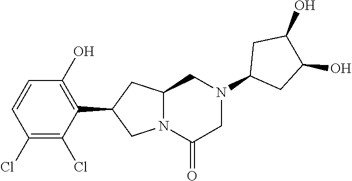 [SMILES: O=C1CN([C@H]2C[C@@H](O)[C@@H](O)C2)C[C@@H]2C[C@H](c3c(O)ccc(Cl)c3Cl)CN12] | (7R,8aS)-7-(2,3- dichloro-6- hydroxyphenyl)-2- [(1R,3R,4S)-rel- 3,4- dihydroxycyclopentyl]- hexahydropyrrolo [1,2-a]pyrazin-4- one | [M + H]⁺: 401, 403 (3:2); ¹H NMR (400 MHz, CD₃OD) δ 7.26 (d, J = 8.8 Hz, 1H), 6.76 (d, J = 8.7 Hz, 1H), 4.40-4.26 (m, 1H), 4.21-4.12 (m, 3H), 3.98-3.89 (m, 1H), 3.69-3.61 (m, 1H), 3.59-3.49 (m, 2H), 3.45-3.38 (m, 1H), 3.13-3.04 (m, 1H), 2.40-2.32 (m, 2H), 2.21-2.14 (m, 1H), 2.06-1.97 (m, 2H), 1.93-1.81 (m, 2H). |
| 131 | 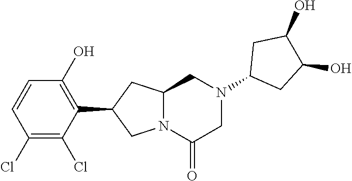 [SMILES: O=C1CN([C@@H]2C[C@@H](O)[C@@H](O)C2)C[C@@H]2C[C@H](c3c(O)ccc(Cl)c3Cl)CN12] | (7R,8aS)-7-(2,3- dichloro-6- hydroxyphenyl)-2- [(1S,3R,4S)-rel- 3,4- dihydroxycyclopentyl]- hexahydropyrrolo [1,2-a]pyrazin-4- one | [M + H]⁺: 401, 403 (3:2); ¹H NMR (400 MHz, CD₃OD) δ 7.26 (d, J = 8.8 Hz, 1H), 6.76 (d, J = 8.8 Hz, 1H), 4.38-4.29 (m, 1H), 4.22-4.12 (m, 1H), 4.00-3.90 (m, 3H), 3.69-3.49 (m, 2H), 3.21-3.03 (m, 2H), 3.41-4.31 (m, 2H), 2.23-2.12 (m, 2H), 1.84-1.76 (m, 2H), 1.86-1.76 (m, 2H). |
| 132 | 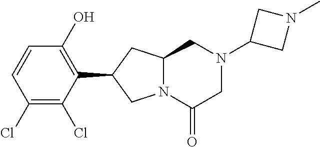 [SMILES: CN1CC(N2CC(=O)N3C[C@@H](c4c(O)ccc(Cl)c4Cl)C[C@H]3C2)C1] | (7R,8aS)-7-(2,3- dichloro-6- hydroxyphenyl)-2- (1-methylazetidin- 3-yl)- hexahydropyrrolo [1,2-a]pyrazin-4- one | [M + H]⁺: 370, 372 (3:2); ¹H NMR (400 MHz, CD₃OD) δ 7.24 (d, J = 8.8 Hz, 1H), 6.74 (d, J = 8.8 Hz, 1H), 4.36-4.26 (m, 1H), 4.15 (dd, J = 11.6, 8.8 Hz, 1H), 3.95-3.85 (m, 1H), 3.66-3.49 (m, 3H), 3.39 (d, J = 16.6 Hz, 1H), 3.23- 3.11 (m, 2H), 3.08-3.03 (m, |
| | | | 2H), 2.76 (d, J = 16.5 Hz, |
| | | | 1H), 2.39 (s, 3H), 2.37-2.29 |
| | | | (m, 1H), 2.17-2.03 (m, 2H). |
| 133 | 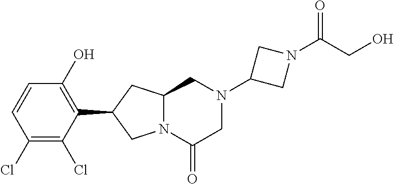 [SMILES: O=C(CO)N1CC(N2CC(=O)N3C[C@@H](c4c(O)ccc(Cl)c4Cl)C[C@H]3C2)C1] | (7R,8aS)-7-(2,3- dichloro-6- hydroxyphenyl)-2- [1-(2- hydroxyacetyl) azetidin-3-yl]- hexahydropyrrolo [1,2-a]pyrazin-4- one | [M + H]⁺: 414, 416 (3:2); ¹H NMR (400 MHz, CD₃OD) δ 7.25 (d, J = 8.8 Hz, 1H), 6.75 (d, J = 8.8 Hz, 1H), 4.40-4.27 (m, 2H), 4.25-4.06 (m, 5H), 3.99-3.90 (m, 2H), 3.61-3.44 (m, 3H), 3.31-3.27 (m, 1H), 2.85 (dd, J = 16.4, 6.3 Hz, 1H), 2.39-2.30 (m, 1H), 2.22- 2.09 (m, 2H). |
| 134 | 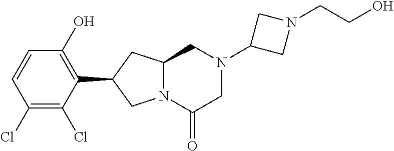 [SMILES: O=C1CN(C2CN(CCO)C2)C[C@@H]2C[C@H](c3c(O)ccc(Cl)c3Cl)CN12] | (7R,8aS)-7-(2,3- dichloro-6- hydroxyphenyl)-2- [1-(2- hydroxyethyl) azetidin-3-yl]- hexahydropyrrolo [1,2-a]pyrazin-4- one | [M + H]⁺: 400, 402 (3:2); ¹H NMR (400 MHz, CD₃OD) δ 7.25 (d, J = 8.8 Hz, 1H), 6.75 (d, J = 8.8 Hz, 1H), 4.38-4.25 (m, 1H), 4.15 (dd, J = 11.6, 8.8 Hz, 1H), 3.96-3.86 (m, 1H), 3.66-3.50 (m, 5H), 3.40 (d, J = 16.5 Hz, 1H), 3.26- 3.15 (m, 2H), 3.13-3.08 (m, 2H), 2.77 (d, J = 16.6 Hz, |
| | | | 1H), 2.67 (t, J = 5.8 Hz, 2H), |
| | | | 2.38-2.29 (m, 1H), 2.17-2.06 |
| | | | (m, 2H). |
| 135 | 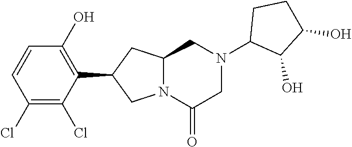 [SMILES: O=C1CN(C2CC[C@H](O)[C@@H]2O)C[C@@H]2C[C@H](c3c(O)ccc(Cl)c3Cl)CN12] | (7R,8aS)-7-(2,3- dichloro-6- hydroxyphenyl)-2- [(2R,3S)-rel-2,3- dihydroxycyclopentyl]- hexahydropyrrolo [1,2-a]pyrazin-4- one | [M + H]⁺: 401,403 (3:2); ¹H NMR (400 MHz, CD₃OD) δ 7.25 (d, J = 8.8 Hz, 1H), 6.75 (d, J = 8.8 Hz, 1H), 4.36-4.27 (m, 1H), 4.16 (dd, J = 11.5, 8.8 Hz, 1H), 4.09 (td, J = 8.0, 3.5 Hz, 1H), 4.01-3.89 (m, 2H), 3.79 (d, J = 16.6 Hz, 1H), 3.55-3.51 (m, 1H), 3.39- 3.36 (m, 1H), 2.93 (d, J = |
| | | | 16.7 Hz, 1H), 2.57 (td, J = |
| | | | 8.4, 3.3 Hz, 1H), 2.36-2.28 |
| | | | (m, 1H), 2.15-2.05 (m, 2H), |
| | | | 2.02-1.76 (m, 4H). |
| 136 | 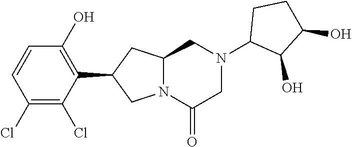 [SMILES: O=C1CN(C2CC[C@@H](O)[C@H]2O)C[C@@H]2C[C@H](c3c(O)ccc(Cl)c3Cl)CN12] | (7R,8aS)-7-(2,3- dichloro-6- hydroxyphenyl)-2- [(2S,3R)-rel-2,3- dihydroxycyclopentyl]- hexahydropyrrolo [1,2-a]pyrazin-4-one | [M + H]⁺: 401, 403 (3:2); ¹H NMR (400 MHz, CD₃OD) δ 7.25 (d, J = 8.8 Hz, 1H), 6.75 (d, J = 8.8 Hz, 1H), 4.36-4.27 (m, 1H), 4.16 (dd, J = 11.5, 8.8 Hz, 1H), 4.12-4.04 (m, 2H), 4.02-3.94 (m, 1H), 3.61- 3.47 (m, 3H), 2.88 (d, J = 16.7 Hz, 1H), 2.62 (td, J = |
| | | | 8.8, 3.4 Hz, 1H), 2.37-2.29 |
| | | | (m, 1H), 2.24-2.18 (m, 1H), |
| | | | 2.15-2.09 (m, 1H), 2.04-1.95 |
| | | | (m, 1H), 1.90-1.75 (m, 3H). |
| 137 | 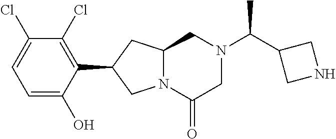 [SMILES: C[C@@H](C1CNC1)N1CC(=O)N2C[C@@H](c3c(O)ccc(Cl)c3Cl)C[C@H]2C1] | (7R,8aS)-2-[1- (azetidin-3- yl)ethyl]-7-(2,3- dichloro-6- hydroxyphenyl)- hexahydropyrrolo [1,2-a]pyrazin-4- one isomer l | [M + H]⁺: 384, 386 (3:2); ¹H NMR (400 MHz, CD₃OD) δ 7.24 (d, J = 8.8 Hz, 1H), 6.74 (d, J = 8.8 Hz, 1H), 4.35-4.25 (m, 1H), 4.14 (dd, J = 11.6, 8.9 Hz, 1H), 3.95-3.85 (m, 1H), 3.83-3.74 (m, 3H), 3.59- 3.47 (m, 2H), 3.23-3.04 (m, 4H), 2.97-2.88 (m, 1H), 2.51- |
| | | | 2.46 (m, 1H), 2.39-2.30 (m, |
| | | | 1H), 2.14-2.07 (m, 1H), 0.98 |
| | | | (d, J = 6.5 Hz, 3H). |
| 138 | 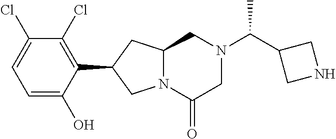 [SMILES: C[C@H](C1CNC1)N1CC(=O)N2C[C@@H](c3c(O)ccc(Cl)c3Cl)C[C@H]2C1] | (7R,8aS)-2-[1- (azetidin-3- yl)ethyl]-7-(2,3- dichloro-6- hydroxyphenyl)- hexahydropyrrolo [1,2-a]pyrazin-4- one isomer 2 | [M + H]⁺: 384, 386 (3:2); ¹H NMR (400 MHz, CD₃OD) δ 7.24 (d, J = 8.8 Hz, 1H), 6.74 (d, J = 8.8 Hz, 1H), 4.35-4.25 (m, 1H), 4.14 (dd, J = 11.6, 8.9 Hz, 1H), 3.87-3.73 (m, 4H), 3.63-3.58 (m, 1H), 3.49 (dd, J = 11.6, 9.9 Hz, 1H), 3.39-3.36 (m, 1H), 3.29-3.26 |
| | | | (m, 1H), 3.20-3.14 (m, 1H), |
| | | | 3.02-2.93 (m, 2H), 2.37-2.28 |
| | | | (m, 1H), 2.23-2.18 (m, 1H), |
| | | | 2.11-2.05 (m, 1H), 0.97 (d, J = |
| | | | 6.5 Hz, 3H). |
| 139 | 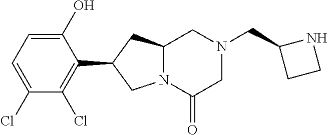 [SMILES: O=C1CN(C[C@@H]2CCN2)C[C@@H]2C[C@H](c3c(O)ccc(Cl)c3Cl)CN12] | (7R,8aS)-2- [azetidin-2- ylmethyl]-7-(2,3- dichloro-6- hydroxyphenyl)- hexahydropyrrolo [1,2-a]pyrazin-4- one isomer 1 | [M + H]⁺: 370, 372 (3:2); ¹H NMR (400 MHz, CD₃OD) δ 8.54 (s, 1H), 7.23 (d, J = 8.8 Hz, 1H), 6.74 (d, J = 8.8 Hz, 1H), 4.72-4.60 (m, 1H), 4.35- 4.25 (m, 1H), 4.17-4.07 (m, 2H), 3.98-3.88 (m, 2H), 3.56- 3.46 (m, 2H), 3.24 (dd, J = 11.1, 4.0 Hz, 1H), 3.12 (dd, J = |
| | | | 13.5, 10.6 Hz, 1H), 2.90 (d, |
| | | | J = 16.5 Hz, 1H), 2.78 (dd, J = |
| | | | 13.5, 4.3 Hz, 1H), 2.61-2.52 |
| | | | (m, 1H), 2.47-2.42 (m, 1H), |
| | | | 2.38-2.29 (m, 2H), 2.14-2.06 |
| | | | (m, 1H). |
| 140 | 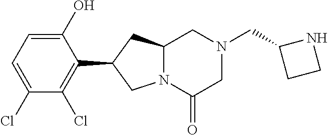 [SMILES: O=C1CN(C[C@H]2CCN2)C[C@@H]2C[C@H](c3c(O)ccc(Cl)c3Cl)CN12] | (7R,8aS)-2- [azetidin-2- ylmethyl]-7-(2,3- dichloro-6- hydroxyphenyl)- hexahydropyrrolo [1,2-a]pyrazin-4- one isomer 2 | [M + H]⁺: 370, 372 (3:2); ¹H NMR (400 MHz, CD₃OD) δ 8.54 (s, 1H), 7.23 (d, J = 8.8 Hz, 1H), 6.74 (d, J = 8.8 Hz, 1H), 4.72-4.60 (m, 1H), 4.35- 4.25 (m, 1H), 4.17-4.07 (m, 2H), 3.98-3.88 (m, 2H), 3.56- 3.46 (m, 3H), 3.18-3.01 (m, 2H), 2.78 (dd, J = 13.5, 4.3 |
| | | | Hz, 1H), 2.61-2.52 (m, 1H), |
| | | | 2.41-2.20 (m, 3H), 2.15-2.06 |
| | | | (m, 1H). |
| 141 | 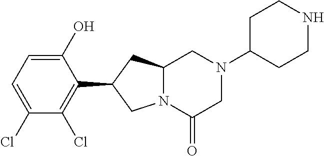 [SMILES: O=C1CN(C2CCNCC2)C[C@@H]2C[C@H](c3c(O)ccc(Cl)c3Cl)CN12] | (7R,8aS)-7-(2,3- dichloro-6- hydroxyphenyl)-2- (piperidin-4-yl)- hexahydropyrrolo [1,2-a]pyrazin-4- one | [M + H]⁺: 384, 386 (3:2); ¹H NMR (400 MHz, CD₃OD) δ 7.27 (d, J = 8.8 Hz, 1H), 6.78 (d, J = 8.8 Hz, 1H), 4.41-4.31 (m, 1H), 4.20 (dd, J = 11.5, 9.1 Hz, 1H), 4.08-4.00 (m, 1H), 3.87-3.76 (m, 2H), 3.61- 3.52 (m, 4H), 3.32-3.23 (m, 1H), 3.16-3.04 (m, 2H), 2.82- 2.77 (m, 1H), 2.49-2.18 (m, |
| | | | 4H), 2.02-1.85 (m, 2H); ¹⁹F |
| | | | NMR (376 MHz, CD₃OD) δ - |
| | | | 77.20 (s, 3H). |
| 142 | 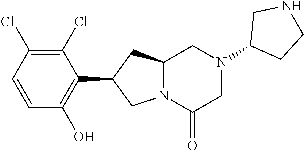 [SMILES: O=C1CN([C@H]2CCNC2)C[C@@H]2C[C@H](c3c(O)ccc(Cl)c3Cl)CN12] | (7R,8aS)-7-(2,3- dichloro-6- hydroxyphenyl)-2- [(3S)-pyrrolidin-3- yl]- hexahydropyrrolo [1,2-a]pyrazin-4- one | [M + H]⁺: 370, 372 (3:2); ¹H NMR (400 MHz, DMSO-d₆ + D₂O) δ 7.29 (d, J = 8.8 Hz, 1H), 6.79 (d, J = 8.8 Hz, 1H), 4.24-4.13 (m, 1H), 4.03-3.90 (m, 1H), 3.87-3.79 (m, 1H), 3.57-3.25 (m, 6H), 3.22-3.00 (m, 3H), 2.41-2.27 (m, 1H), 2.24-2.05 (m, 3H), 1.96-1.84 (m, 1H). |
| 143 | 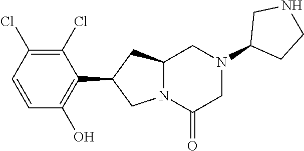 [SMILES: O=C1CN([C@@H]2CCNC2)C[C@@H]2C[C@H](c3c(O)ccc(Cl)c3Cl)CN12] | (7R,8aS)-7-(2,3- dichloro-6- hydroxyphenyl)-2- [(3R)-pyrrolidin-3- yl]- hexahydropyrrolo [1,2-a]pyrazin-4- one | [M + H]⁺: 370, 372 (3:2); ¹H NMR (400 MHz, DMSO-d₆ + D₂O) δ 7.32 (d, J = 8.8 Hz, 1H), 6.82 (d, J = 8.8 Hz, 1H), 4.19-4.10 (m, 1H), 3.98-3.92 (m, 1H), 3.89-3.79 (m, 1H), 3.58-3.29 (m, 6H), 3.22-3.10 (m, 3H), 2.41-2.36 (m, 1H), 2.25-2.04 (m, 3H), 2.00-1.91 (m, 1H). |
| 144 | 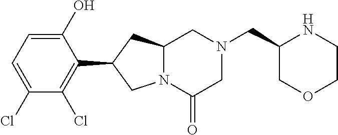 [SMILES: O=C1CN(C[C@@H]2COCCN2)C[C@@H]2C[C@H](c3c(O)ccc(Cl)c3Cl)CN12] | (7R,8aS)-7-(2,3- dichloro-6- hydroxyphenyl)-2- [(3R)-morpholin-3- ylmethyl]- hexahydropyrrolo [1,2-a]pyrazin-4- one | [M + H]⁺: 400, 402 (3:2); ¹H NMR (400 MHz, CD₃OD) δ 7.25 (d, J = 8.8 Hz, 1H), 6.75 (d, J = 8.9 Hz, 1H), 4.38-4.24 (m, 1H), 4.15 (dd, J = 11.6, 8.8 Hz, 1H), 4.00-3.91 (m, 1H), 3.87-3.79 (m, 2H), 3.60- 3.46 (m, 3H), 3.28-3.18 (m, 2H), 3.05-2.98 (m, 1H), 2.94- |
| | | | 2.83 (m, 3H), 2.49 (dd, J = |
| | | | 12.5, 8.4 Hz, 1H), 2.40 (dd, J = |
| | | | 12.5, 5.4 Hz, 1H), 2.35-2.28 |
| | | | (m, 2H), 2.15-2.08 (m, 1H). |
| 145 | 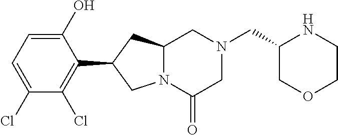 [SMILES: O=C1CN(C[C@H]2COCCN2)C[C@@H]2C[C@H](c3c(O)ccc(Cl)c3Cl)CN12] | (7R,8aS)-7-(2,3- dichloro-6- hydroxyphenyl)-2- [(3S)-morpholin-3- ylmethyl]- hexahydropyrrolo [1,2-a]pyrazin-4- one | [M + H]⁺: 400, 402 (3:2); ¹H NMR (400 MHz, CD₃OD) δ 7.25 (d, J = 8.8 Hz, 1H), 6.75 (d, J = 8.8 Hz, 1H), 4.38-4.24 (m, 1H), 4.15 (dd, J = 11.6, 8.8 Hz, 1H), 4.00-3.91 (m, 1H), 3.87-3.79 (m, 2H), 3.60- 3.46 (m, 3H), 3.27-3.20 (m, 2H), 3.08-3.01 (m, 1H), 3.00- |
| | | | 2.89 (m, 3H), 2.45-2.38 (m, |
| | | | 2H), 2.38-2.28 (m, 1H), 2.25- |
| | | | 2.16 (m, 1H), 2.15-2.08 (m, |
| | | | 1H). |
| 146 | 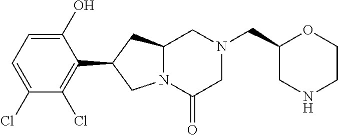 [SMILES: O=C1CN(C[C@@H]2CNCCO2)C[C@@H]2C[C@H](c3c(O)ccc(Cl)c3Cl)CN12] | (7R,8aS)-7-(2,3- dichloro-6- hydroxyphenyl)-2- [(2S)-morpholin-2- ylmethyl]- hexahydropyrrolo [1,2-a]pyrazin-4- one | [M + H]⁺: 400, 402 (3:2); ¹H NMR (400 MHz, CD₃OD) δ 7.25 (d, J = 8.8 Hz, 1H), 6.75 (d, J = 8.8 Hz, 1H), 4.38-4.24 (m, 1H), 4.15 (dd, J = 11.6, 8.8 Hz, 1H), 3.98-3.83 (m, 2H), 3.75-3.67 (m, 1H), 3.65- 3.49 (m, 4H), 3.01-2.87 (m, 2H), 2.87-2.78 (m, 2H), 2.67- |
| | | | 2.45 (m, 3H), 2.38-2.19 (m, |
| | | | 2H), 2.14-2.06 (m, 1H). |
| 147 | 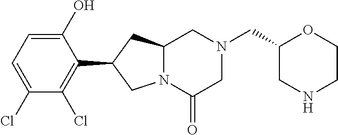 [SMILES: O=C1CN(C[C@H]2CNCCO2)C[C@@H]2C[C@H](c3c(O)ccc(Cl)c3Cl)CN12] | (7R,8aS)-7-(2,3- dichloro-6- hydroxyphenyl)-2- [(2R)-morpholin-2- ylmethyl]- hexahydropyrrolo [1,2-a]pyrazin-4- one | [M + H]⁺: 400, 402 (3:2); ¹H NMR (400 MHz, CD₃OD) δ 7.26 (d, J = 8.8 Hz, 1H), 6.76 (d, J = 8.8 Hz, 1H), 4.37-4.26 (m, 1H), 4.21-4.08 (m, 2H), 3.99-3.89 (m, 2H), 3.86-3.77 (m, 1H), 3.61 (d, J = 16.8 Hz, 1H), 3.52 (t, J = 10.8 Hz, 1H), 3.39-3.32 (m, 2H), 3.31-3.27 |
| | | | (m, 1H), 3.22-3.13 (m, 1H), |
| | | | 3.10-2.96 (m, 2H), 2.77 (dd, J = |
| | | | 13.6, 6.0 Hz, 1H), 2.66 (dd, |
| | | | J = 13.6, 4.5 Hz, 1H), 2.38- |
| | | | 2.29 (m, 2H), 2.16-2.04 (m, |
| | | | 1H). |

### Example 47. Compound 148 ((7R,8aS)-7-(2,3-dichloro-6-hydroxyphenyl)-2-(2-hydroxy-2-methylpropyl)-hexahydropyrrolo[1,2-a]pyrazin-4-one)

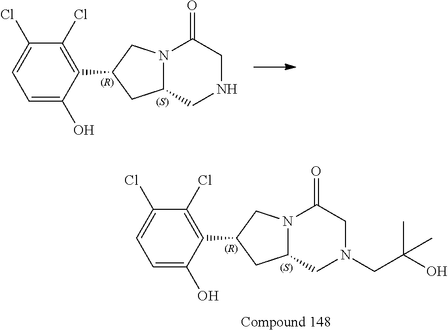

To a stirred solution of (7R,8aS)-7-(2,3-dichloro-6-hydroxyphenyl)-hexahydro-1H-pyrrolo[1,2-a]pyrazin-4-one (Intermediate 8 free base, Example 7) (30.0 mg, 0.10 mmol) in EtOH (1 mL) was added 2,2-dimethyloxirane (11.0 mg, 0.15 mmol) at room temperature under nitrogen. The resulting solution was stirred at 80° C. for 36 h, then concentrated under reduced pressure. The residue was purified by Prep-HPLC with the following conditions Column: X Bridge Shield RP18 OBD Column, 30×150 mm, 5 m; Mobile Phase A: water (plus 10 mM NH₄HCO₃, Mobile Phase B: ACN; Flow rate: 60 mL/min; Gradient: 25% B to 45% B in 7 min; Detector: UV 220 nm; Retention time: 7.12 min. The fractions containing the desired product were collected and concentrated under reduced pressure to afford (7R,8aS)-7-(2,3-dichloro-6-hydroxyphenyl)-2-(2-hydroxy-2-methylpropyl)-hexahydropyrrolo[1,2-a]pyrazin-4-one as an off-white solid (20 mg, 53%). LCMS (ESI) calc'd C₁₇H₂₂Cl₂N₂O₃ for [M+H]⁺: 373, 375 (3:2) found 373, 375 (3:2); ¹H NMR (400 MHz, CD₃OD) δ 7.25 (d, J=8.8 Hz, 1H), 6.75 (d, J=8.9 Hz, 1H), 4.38-4.24 (m, 1H), 4.15 (t, J=11.5 Hz, 1H), 4.06-3.92 (m, 1H), 3.64 (d, J=17.0 Hz, 1H), 3.52 (t, J=10.7 Hz, 1H), 3.40 (dd, J=11.8, 3.8 Hz, 1H), 3.05 (d, J=17.1 Hz, 1H), 2.57-2.40 (m, 2H), 2.40-2.22 (m, 2H), 2.14-2.02 (m, 1H), 1.24 (s, 6H).

Example 48. Compounds 149-159 were prepared in an analogous fashion as that described for Compound 148.

| Compound | | Chemical | |
|---|---|---|---|
| Number | Structure | Name | MS: (M + H)⁺ & ¹H MNR |
| 149 | 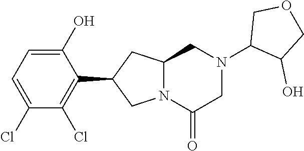 [SMILES: O=C1CN(C2COCC2O)C[C@@H]2C[C@H](c3c(O)ccc(Cl)c3Cl)CN12] | (7R,8aS)-7- (2,3-dichloro-6- hydroxyphenyl)- 2-(4- hydroxyoxolan- 3-yl)- hexahydropyrrolo[1,2- a]pyrazin-4-one | [M + H]⁺: 387, 389 (3:2); ¹H NMR (400 MHz, CD₃OD) δ 7.27 (d, J = 8.8 Hz, 1H), 6.77 (d, J = 8.8 Hz, 1H), 4.56-4.47 (m, 1H), 4.42-4.28 (m, 1H), 4.26- 4.16 (m, 1H), 4.16-4.05 (m, 2H), 4.04-3.80 (m, 3H), 3.79-3.54 (m, 3H), 3.53- 3.42 (m, 1H), 3.39-3.34 (m, |
| | | | 1H), 2.81-2.69 (m, 1H), |
| | | | 2.47-2.34 (m, 1H), 2.26- |
| | | | 2.15 (m, 1H). |
| 150 | 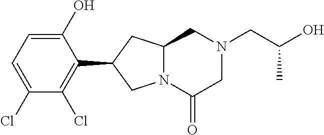 [SMILES: C[C@@H](O)CN1CC(=O)N2C[C@@H](c3c(O)ccc(Cl)c3Cl)C[C@H]2C1] | (7R,8aS)-7- (2,3-dichloro-6- hydroxyphenyl)- 2-[2- hydroxypropyl] hexahydropyrrolo[1,2- a]pyrazin-4-one isomer 1 | [M + H]⁺: 359, 361 (3:2); ¹H NMR (400 MHz, CD₃OD) δ 7.32-7.23 (m, 1H), 6.79 (d, J = 8.8 Hz, 1H), 4.45-4.31 (m, 1H), 4.30-4.00 (m, 5H), 3.97- 3.85 (m, 1H), 3.63 (t, J = 10.6 Hz, 1H), 3.31-3.12 (m, 3H), 2.49-2.45 (m, 1H), |
| | | | 2.33-2.23 (m, 1H), 1.28 (d, |
| | | | J = 6.1 Hz, 3H). |
| 151 | 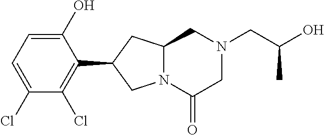 [SMILES: C[C@H](O)CN1CC(=O)N2C[C@@H](c3c(O)ccc(Cl)c3Cl)C[C@H]2C1] | (7R,8aS)-7- (2,3-dichloro-6- hydroxyphenyl)- 2-[2- hydroxypropyl] hexahydropyrrolo[1,2- a]pyrazin-4-one isomer 2 | [M + H]⁺: 359, 361 (3:2); ¹H NMR (400 MHz, CD₃OD) δ 7.29 (dd, J = 8.8, 1.5 Hz, 1H), 6.79 (d, J = 8.8 Hz, 1H), 4.44-4.31 (m, 1H), 4.26-4.00 (m, 5H), 4.00-3.87 (m, 1H), 3.64 (t, J = 10.6 Hz, 1H), 3.32-3.19 (m, 3H), 2.49-2.45 (m, 1H), |
| | | | 2.32-2.21 (m, 1H), 1.27 (d, |
| | | | J = 6.2 Hz, 3H). |
| 152 | 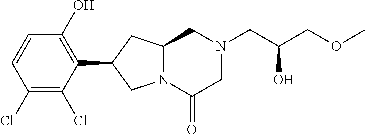 [SMILES: COC[C@@H](O)CN1CC(=O)N2C[C@@H](c3c(O)ccc(Cl)c3Cl)C[C@H]2C1] | (7R,8aS)-7- (2,3-dichloro-6- hydroxyphenyl)- 2-[2-hydroxy- 3- methoxypropyl] hexahydropyrrolo[1,2- a]pyrazin-4-one isomer 1 | [M + H]⁺: 389, 391 (3:2); ¹H NMR (400 MHz, CD₃OD) δ 7.29 (d, J = 8.8 Hz, 1H), 6.79 (d, J = 8.8 Hz, 1H), 4.46-4.32 (m, 1H), 4.26-4.17 (m, 2H), 4.17- 4.05 (m, 3H), 3.95-3.83 (m, 1H), 3.63 (d, J = 11.2 Hz, 1H), 3.54-3.43 (m, 2H), |
| | | | 3.41 (s, 3H), 3.38-3.34 (m, |
| | | | 2H), 3.24-3.13 (m, 1H), |
| | | | 2.49-2.45 (m, 1H), 2.34- |
| | | | 2.21 (m, 1H). |
| 153 | 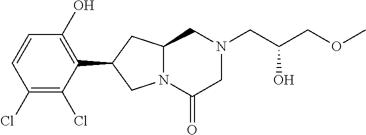 [SMILES: COC[C@H](O)CN1CC(=O)N2C[C@@H](c3c(O)ccc(Cl)c3Cl)C[C@H]2C1] | (7R,8aS)-7- (2,3-dichloro-6- hydroxyphenyl)- 2-[2-hydroxy- 3- methoxypropyl] hexahydropyrrolo[1,2- a]pyrazin-4-one isomer 2 | [M + H]⁺: 389, 391 (3:2); ¹H NMR (400 MHz, CD₃OD) δ 7.29 (dd, J = 8.8, 1.0 Hz, 1H), 6.79 (d, J = 8.8 Hz, 1H), 4.45-4.32 (m, 1H), 4.28-3.99 (m, 5H), 3.98-3.85 (m, 1H), 3.67- 3.60 (m, 1H), 3.53-3.43 (m, 2H), 3.42 (s, 3H), 3.41-3.37 |
| | | | (m, 2H), 3.31-3.26 (m, 1H), |
| | | | 2.49-2.46 (m, 1H), 2.34- |
| | | | 2.20 (m, 1H). |
| 154 | 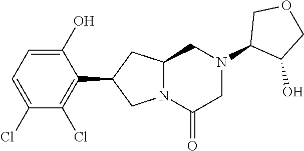 [SMILES: O=C1CN([C@H]2COC[C@@H]2O)C[C@@H]2C[C@H](c3c(O)ccc(Cl)c3Cl)CN12] | (7R,8aS)-7-(2,3- dichloro-6- hydroxyphenyl)- 2-[(3S,4R)-rel- 4- hydroxyoxolan- 3-yl]- hexahydropyrrolo [1,2-a]pyrazin- 4-one | [M + H]⁺: 387, 389 (3:2); ¹H NMR (400 MHz, CD₃OD) δ 7.25 (d, J = 8.7 Hz, 1H), 6.75 (d, J = 8.8 Hz, 1H), 4.39-4.24 (m, 2H), 4.17 (dd, J = 11.5, 8.8 Hz, 1H), 4.09 (dd, J = 9.4, 6.6 Hz, 1H), 3.98 (dd, J = 9.6, 5.9 Hz, 1H), 3.94-3.85 (m, 1H), 3.76 (dd, J = 9.4, 5.8 |
| | | | Hz, 1H), 3.70 (d, J = 16.9 |
| | | | Hz, 1H), 3.65 (dd, J = 9.6, |
| | | | 3.9 Hz, 1H), 3.55-3.48 (m, |
| | | | 1H), 3.24-3.19 (m, 1H), |
| | | | 3.05 (d, J = 16.9 Hz, 1H), |
| | | | 2.98-2.92 (m, 1H), 2.37- |
| | | | 2.23 (m, 2H), 2.16-2.07 (m, |
| | | | 1H). |
| 155 | 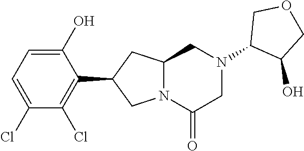 [SMILES: O=C1CN([C@@H]2COC[C@H]2O)C[C@@H]2C[C@H](c3c(O)ccc(Cl)c3Cl)CN12] | (7R,8aS)-7- (2,3-dichloro-6- hydroxyphenyl)- 2-[(3R,4S)-rel- 4- hydroxyoxolan- 3-yl]- hexahydropyrrolo[1,2- a]pyrazin-4-one | [M + H]⁺: 387, 389 (3:2); ¹H NMR (400 MHz, CD₃OD) δ 7.25 (d, J = 8.8 Hz, 1H), 6.76 (d, J = 8.8 Hz, 1H), 4.39-4.26 (m, 1H), 4.17 (dd, J = 11.5, 8.8 Hz, 1H), 4.08 (dd, J = 9.5, 6.7 Hz, 1H), 3.99 (dd, J = 9.7, 5.8 Hz, 1H), 3.95-3.86 (m, 1H), 3.73 (dd, J = 9.5, 5.8 |
| | | | Hz, 1H), 3.66 (dd, J = 9.7, |
| | | | 3.5 Hz, 1H), 3.64-3.54 (m, |
| | | | 2H), 3.53-3.48 (m, 1H), |
| | | | 3.48-3.39 (m, 1H), 3.06 (d, |
| | | | J = 16.6 Hz, 1H), 3.01-2.95 |
| | | | (m, 1H), 2.40-2.24 (m, 2H), |
| | | | 2.17-2.09 (m, 1H). |
| 156 | 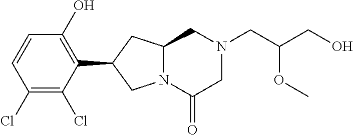 [SMILES: COC(CO)CN1CC(=O)N2C[C@@H](c3c(O)ccc(Cl)c3Cl)C[C@H]2C1] | (7R,8aS)-7- (2,3-dichloro-6- hydroxyphenyl)- 2-(3-hydroxy- 2- methoxypropyl) hexahydropyrrolo[1,2- a]pyrazin-4-one | [M + H]⁺: 389, 391 (3:2); ¹H NMR (400 MHz, CD₃OD) δ 7.25 (d, J = 8.8 Hz, 1H), 6.75 (d, J = 8.8 Hz, 1H), 4.39-4.25 (m, 1H), 4.15 (dd, J = 11.6, 8.8 Hz, 1H), 3.98-3.88 (m, 1H), 3.71 (dt, J = 11.7, 4.3 Hz, 1H), 3.65-3.56 (m, 2H), |
| | | | 3.56-3.42 (m, 5H), 3.42- |
| | | | 3.36 (m, 1H), 2.96 (d, J = |
| | | | 16.8 Hz, 1H), 2.74-2.59 (m, |
| | | | 2H), 2.37-2.25 (m, 2H), |
| | | | 2.16-2.06 (m, 1H). |
| 157 | 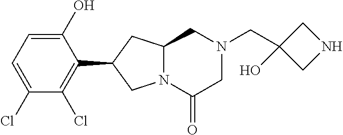 [SMILES: O=C1CN(CC2(O)CNC2)C[C@@H]2C[C@H](c3c(O)ccc(Cl)c3Cl)CN12] | (7R,8aS)-7- (2,3-dichloro-6- hydroxyphenyl)- 2-[(3- hydroxyazetidin- 3-yl)methyl]- hexahydropyrrolo[1,2- a]pyrazin-4- one; | [M + H]⁺: 386, 388 (3:2); ¹H NMR (400 MHz, CD₃OD) δ 7.26 (d, J = 8.8 Hz, 1H), 6.77 (d, J = 8.8 Hz, 1H), 4.41-4.24 (m, 1H), 4.23-4.13 (m, 3H), 4.09- 3.97 (m, 3H), 3.83-3.69 (m, 1H), 3.62-3.46 (m, 2H), 3.30-3.24 (m, 1H), 3.19- |
| | | | 2.92 (m, 2H), 2.79-2.58 (m, |
| | | | 1H), 2.44-2.29 (m, 1H), |
| | | | 2.22-2.09 (m, 1H). |
| 158 | 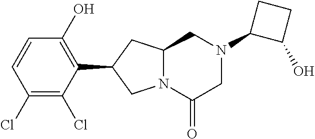 [SMILES: O=C1CN([C@H]2CC[C@@H]2O)C[C@@H]2C[C@H](c3c(O)ccc(Cl)c3Cl)CN12] | (7R,8aS)-7- (2,3-dichloro-6- hydroxyphenyl)- 2-[(1R,2R)-rel- 2- hydroxycyclobutyl]- hexahydropyrrolo[1,2- a]pyrazin-4-one | [M + H]⁺: 371, 373 (3:2); ¹H NMR (400 MHz, CD₃OD) δ 7.25 (d, J = 8.8 Hz, 1H), 6.75 (d, J = 8.8 Hz, 1H), 4.37-4.25 (m, 1H), 4.16 (dd, J = 11.6, 8.7 Hz, 1H), 4.00-3.98 (m, 1H), 3.95-3.84 (m, 1H), 3.62 (d, J = 17.1 Hz, 1H), 3.56-3.47 |
| | | | (m, 1H), 3.30-3.23 (m, 1H), |
| | | | 2.90 (d, J = 17.0 Hz, 1H), |
| | | | 2.79-2.70 (m, 1H), 2.35- |
| | | | 2.31 (m, 1H), 2.20-2.07 (m, |
| | | | 3H), 1.95-1.91 (m, 1H), |
| | | | 1.64-1.50 (m, 1H), 1.38- |
| | | | 1.26 (m, 1H). |
| 159 | 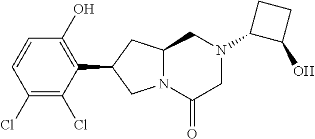 [SMILES: O=C1CN([C@@H]2CC[C@H]2O)C[C@@H]2C[C@H](c3c(O)ccc(Cl)c3Cl)CN12] | (7R,8aS)-7- (2,3-dichloro-6- hydroxyphenyl)- 2-[(1S,2S)-rel- 2- hydroxycyclobutyl]- hexahydropyrrolo[1,2- a]pyrazin-4-one | [M + H]⁺: 371, 373 (3:2); ¹H NMR (400 MHz, CD₃OD) δ 7.29 (d, J = 8.8 Hz, 1H), 6.79 (d, J = 8.8 Hz, 1H), 4.44-4.33 (m, 1H), 4.33-4.18 (m, 2H), 4.18- 4.06 (m, 1H), 4.03-3.89 (m, 2H), 3.76-3.67 (m, 1H), 3.63 (t, J = 11.5 Hz, 1H), |
| | | | 3.57-3.45 (m, 1H), 3.17- |
| | | | 3.07 (m, 1H), 2.49-2.45 (m, |
| | | | 1H), 2.36-2.23 (m, 2H), |
| | | | 2.23-2.09 (m, 1H), 1.78- |
| | | | 1.55 (m, 2H). |

### Example 49. Compound 160(4-[(7R,8aS)-7-(2,3-dichloro-6-hydroxyphenyl)-4-oxo-hexahydropyrrolo[1,2-a]pyrazin-2-yl]pyrrolidine-2-one isomer 1) and Compound 161 (4-[(7R,8aS)-7-(2,3-dichloro-6-hydroxyphenyl)-4-oxo-hexahydropyrrolo[1,2-a]pyrazin-2-yl]pyrrolidin-2-one isomer 2)

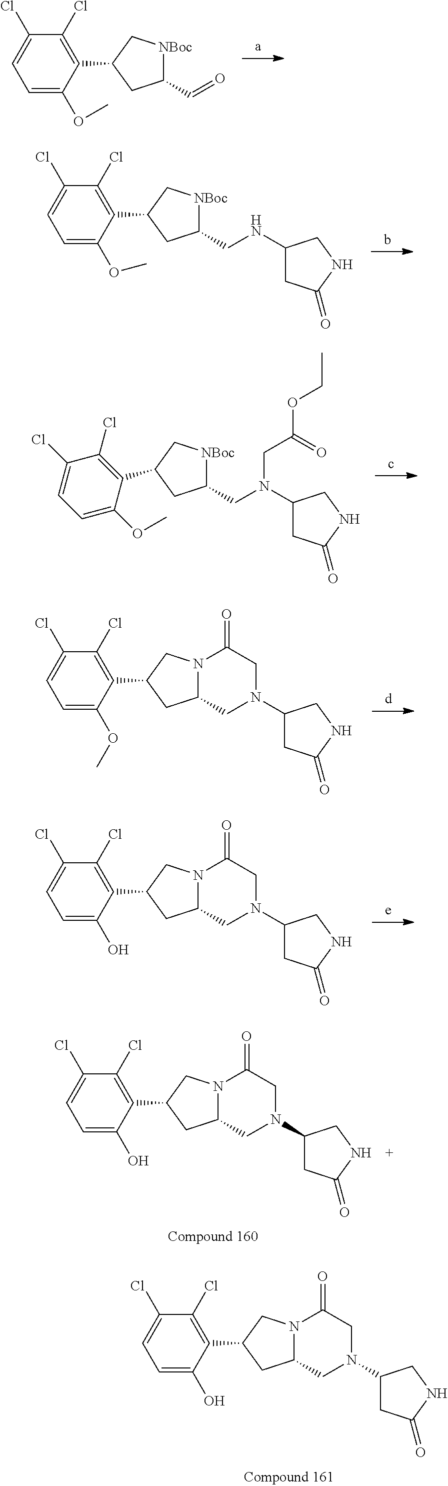

Step a:

To a stirred solution of tert-butyl (2S,4R)-4-(2,3-dichloro-6-methoxyphenyl)-2-formylpyrrolidine-1-carboxylate (Example 7, step c) (0.500 g, 1.34 mmol) and 4-aminopyrrolidin-2-one (0.550 g, 4.01 mmol) in DCM (10 mL) were added TEA (0.540 g, 5.34 mmol) and NaBH(OAc)₃ (1.13 g, 5.34 mmol) at room temperature. The reaction was stirred for 12 h monitoring by LCMS, quenched with saturated aq. NH₄Cl (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 75% ACN in water (plus 10 mM NH₄HCO₃) to afford tert-butyl (2S,4R)-4-(2,3-dichloro-6-methoxyphenyl)-2-[[(5-oxopyrrolidin-3-yl)amino]methyl]pyrrolidine-1-carboxylate as an off-white solid (0.450 g, 73%): LCMS (ESI) calc'd for C₂₁H₂₉Cl₂N₃O₄ [M+H]⁺: 458, 460 (3:2) found 458, 460 (3:2); H NMR (400 MHz, CD₃OD) δ 7.42 (d, J=8.9 Hz, 1H), 7.00 (d, J=9.0 Hz, 1H), 4.15-3.97 (m, 2H), 3.89 (s, 3H), 3.85-3.53 (m, 3H), 3.26-3.14 (m, 1H), 3.14-3.03 (m, 1H), 2.81-2.72 (m, 1H), 2.72-2.56 (m, 1H), 2.54-2.40 (m, 1H), 2.39-2.11 (m, 2H), 1.83-1.64 (m, 1H), 1.51 (s, 9H).

Step b:

To a stirred solution of tert-butyl (2S,4R)-4-(2,3-dichloro-6-methoxyphenyl)-2-[[(5-oxopyrrolidin-3-yl)amino]methyl]pyrrolidine-1-carboxylate (0.150 g, 0.33 mmol) and ethyl bromoacetate (0.110 g, 0.65 mmol,) in ACN (5 mL) were added K₂CO₃ (90.5 mg, 0.65 mmol) at room temperature. The reaction was stirred at 80° C. for 2 h. The cooled solution was diluted with EA (20 mL) and water (30 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 29% ACN in water (plus 0.05% TFA) to afford tert-butyl (2S,4R)-4-(2,3-dichloro-6-methoxyphenyl)-2-[[(2-ethoxy-2-oxoethyl)(5-oxopyrrolidin-3-yl)amino]methyl]pyrrolidine-1-carboxylate as an colorless oil (0.120 g, 67%): LCMS (ESI) calc'd for C₂₅H₃₅Cl₂N₃O₆ [M+H]⁺: 544, 546 (3:2) found 544, 546 (3:2); ¹H NMR (400 MHz, CD₃OD) δ 7.45 (d, J=9.0 Hz, 1H), 7.02 (d, J=9.0 Hz, 1H), 4.66 (d, J=27.0 Hz, 1H), 4.46 (t, J=8.2 Hz, 1H), 4.40-4.29 (m, 2H), 4.29-4.04 (m, 3H), 3.90 (s, 3H), 3.85-3.57 (m, 3H), 3.57-3.41 (m, 2H), 3.03-2.57 (m, 2H), 2.51-2.28 (m, 2H), 1.83-1.61 (m, 1H), 1.54 (s, 9H), 1.36 (t, J=6.9 Hz, 3H).

Step c:

A solution of tert-butyl (2S,4R)-4-(2,3-dichloro-6-methoxyphenyl)-2-[[(2-ethoxy-2-oxoethyl)(5-oxopyrrolidin-3-yl)amino]methyl]pyrrolidine-1-carboxylate (0.120 g, 0.220 mmol) and TFA (1.50 mL, 1.346 mmol) in DCM (3.00 mL) was stirred at room temperature for 1 h. The reaction was concentrated under reduced pressure. The residue was dissolved in EtOH (3.00 mL) and TEA (1.00 mL) added. The solution was stirred at 80° C. for 2 h. The cooled solution was diluted with EA (20 mL) and water (30 mL) and extracted with more EA (3×20 mL). The combined organic layers were washed with brine (3×20 mL) and dried over anhydrous Na₂SO₄. After filtration, the filtrate was concentrated under reduced pressure to afford (7R,8aS)-7-(2,3-dichloro-6-methoxyphenyl)-2-(5-oxopyrrolidin-3-yl)hexahydropyrrolo[1,2-a]pyrazin-4(1H)-one as a yellow oil (0.100 g, crude), which was used to next step directly without purification: LCMS (ESI) calc'd for C₁₈H₂₁Cl₂N₃O₃ [M+H]⁺: 398, 400 (3:2) found 398, 400 (3:2).

Step d:

To a stirred solution of (7R,8aS)-7-(2,3-dichloro-6-methoxyphenyl)-2-(5-oxopyrrolidin-3-yl)hexahydropyrrolo[1,2-a]pyrazin-4(1H)-one (0.100 g, 0.25 mmol) in DCM (2.00 mL) was added BBr₃ (0.330 g, 1.32 mmol) at room temperature. The reaction was stirred for 1 h, quenched with MeOH (5 mL) and concentrated under reduced pressure. The residue was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 10% to 40% in 8 min; Detector: UV 254/220 nm; Retention time: 6.28 min. The fractions containing desired product were collected and concentrated under reduced pressure to afford 4-[(7R,8aS)-7-(2,3-dichloro-6-hydroxyphenyl)-4-oxo-hexahydropyrrolo[1,2-a]pyrazin-2-yl]pyrrolidin-2-one as an off-white solid (31.0 mg, 36.47% overall two steps): LCMS (ESI) calc'd for C₁₇H₁₉Cl₂N₃O₃ [M+H]⁺: 384, 386 (3:2) found 384, 386 (3:2); ¹H NMR (400 MHz, DMSO-d₆) δ 7.36 (d, J=8.8 Hz, 1H), 6.85 (d, J=8.8 Hz, 1H), 4.24-4.10 (m, 1H), 4.07-3.85 (m, 4H), 3.81-3.69 (m, 1H), 3.63-3.57 (m, 2H), 3.51-3.38 (m, 2H), 2.96 (t, J=11.3 Hz, 1H), 2.65-2.54 (m, 2H), 2.26-2.08 (m, 2H).

Step e:

4-[(7R,8aS)-7-(2,3-dichloro-6-hydroxyphenyl)-4-oxo-hexahydropyrrolo[1,2-a]pyrazin-2-yl]pyrrolidin-2-one (30.0 mg, 0.08 mmol) was separated by Prep Chiral HPLC with the following conditions: Column: CHIRALPAK IG, 2×25 cm, 5 μm; Mobile Phase A: Hex (plus 0.2% IPA)-HPLC, Mobile Phase B: EtOH-HPLC; Flow rate: 20 mL/min; Gradient: 50% to 50% in 22 min; Detector: UV 254/220 nm; Retention time 1:10.99 min; Retention time 2: 17.77 min. The faster-eluting isomer at 10.99 min was obtained Compound 160 (4-[(7R,8aS)-7-(2,3-dichloro-6-hydroxyphenyl)-4-oxo-hexahydropyrrolo[1,2-a]pyrazin-2-yl]pyrrolidin-2-one isomer 1) as an off-white solid (6 mg, 20%): LCMS (ESI) calc'd for C₁₇H₁₉Cl₂N₃O₃ [M+H]⁺: 384, 386 (3:2) found 384, 386 (3:2); ¹H NMR (400 MHz, CD₃OD) δ 7.25 (d, J=8.8 Hz, 1H), 6.76 (d, J=8.8 Hz, 1H), 4.40-4.25 (m, 1H), 4.16 (dd, J=11.6, 8.9 Hz, 1H), 3.99-3.87 (m, 1H), 3.60 (dd, J=9.9, 7.4 Hz, 1H), 3.55-3.41 (m, 4H), 3.36-3.34 (m, 1H), 2.99 (d, J=16.5 Hz, 1H), 2.54 (dd, J=16.8, 8.1 Hz, 1H), 2.45-2.30 (m, 2H), 2.27 (dd, J=11.5, 10.1 Hz, 1H), 2.18-2.09 (m, 1H); the slower-eluting isomer at 17.77 min was obtained Compound 161 (4-[(7R,8aS)-7-(2,3-dichloro-6-hydroxyphenyl)-4-oxo-hexahydropyrrolo[1,2-a]pyrazin-2-yl]pyrrolidin-2-one isomer 2) as an off-white solid (4.7 mg, 15.6739): LCMS (ESI) calc'd for C₁₇H₁₉Cl₂N₃O₃ [M+H]⁺: 384, 386 (3:2) found 384, 386 (3:2); ¹H NMR (400 Hz, CD₃OD) δ 7.25 (d, J=8.8 Hz, 1H), 6.76 (d, J=8.7 Hz, 1H), 4.37-4.24 (m, 1H), 4.16 (dd, J=11.4, 8.9 Hz, 1H), 3.96-3.84 (m, 1H), 3.61 (dd, J=9.7, 7.3 Hz, 1H), 3.57-3.41 (m, 3H), 3.38-3.35 (di, 1H), 3.28-3.21 (m, 1H), 3.00 (d, J=16.6 Hz, 1H), 2.54 (dd, J=16.8, 8.1 Hz, 1H), 2.44-2.22 (m, 3H), 2.17-2.09 (m, 1H).

Example 50. Compounds 162-167 were prepared in an analogous fashion as that described for Compounds 160 and 161.

| Compound | | | |
|---|---|---|---|
| Number | Structure | Chemical Name | MS: (M + H)⁺ & ¹H MNR |
| 162 | 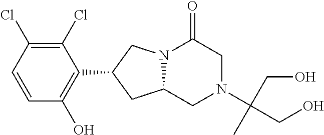 [SMILES: CC(CO)(CO)N1CC(=O)N2C[C@@H](c3c(O)ccc(Cl)c3Cl)C[C@H]2C1] | (7R,8aS)-7-(2,3- dichloro-6- hydroxyphenyl)-2- (1,3-dihydroxy-2- methylpropan-2-yl)- hexahydropyrrolo[1, 2-a]pyrazin-4-one | [M + H]⁺: 389, 391 (3:2); ¹H NMR (400 MHz, CD₃OD) δ 7.29 (d, J = 8.8 Hz, 1H), 6.79 (d, J = 8.8 Hz, 1H), 4.45-4.35 (m, 1H), 4.35-4.25 (m, 1H), 4.25-4.03 (m, 4H), 3.94 (dd, J = 37.7, 12.6 Hz, 2H), 3.77 (dd, J = 12.6, 1.9 Hz, 2H), 3.63 (dd, J = 11.4, 9.7 Hz, 1H), 3.32-3.27 (m, 1H), 2.51- |
| | | | 2.46 (m, 1H), 2.35-2.26 (m, 1H), |
| | | | 1.41 (s, 3H). |
| 163 | 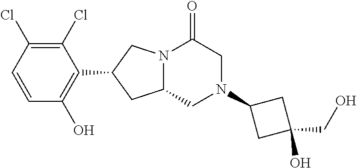 [SMILES: O=C1CN([C@H]2C[C@](O)(CO)C2)C[C@@H]2C[C@H](c3c(O)ccc(Cl)c3Cl)CN12] | (7R,8aS)-1-(2,3- dichloro-6- hydroxyphenyl)-2- [(3R)-3-hydroxy-3- (hydroxymethyl) cyclobutyl]- hexahydropyrrolo [1,2-a]pyrazin-4-one isomer 1 | [M + H]⁺: 401, 403 (3:2); ¹H NMR (400 MHz, CD₃OD) δ 7.25 (d, J = 8.8 Hz, 1H), 6.75 (d, J = 8.8 Hz, 1H), 4.38-4.25 (m, 1H), 4.19-4.10 (m, 1H), 3.96-3.84 (m, 1H), 3.57-3.41 (m, 4H), 3.28 (dd, J = 3.8, 1.4 Hz, 1H), 2.73 (d, J = 16.5 Hz, 1H), 2.61-2.51 (m, 1H), 2.47-2.36 (m, 2H), 2.36-2.26 (m, 1H), 2.17-2.08 (m, 1H), 2.04 (t, J = 11.5 Hz, 1H), 1.97-1.88 (m, |
| | | | 2H). |
| 164 | 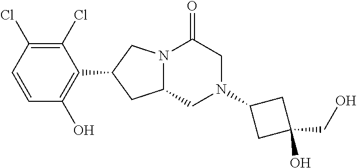 [SMILES: O=C1CN([C@H]2C[C@@](O)(CO)C2)C[C@@H]2C[C@H](c3c(O)ccc(Cl)c3Cl)CN12] | (7R,8aS)-1-(2,3- dichloro-6- hydroxyphenyl)-2- [(3S)-3-hydroxy-3- (hydroxymethyl) cyclobutyl]- hexahydropyrrolo[1, 2-a]pyrazin-4-one isomer 2 | [M + H]⁺: 401, 403 (3:2); ¹H NMR (400 MHz, CD₃OD) δ 7.25 (d, J = 8.8 Hz, 1H), 6.75 (d, J = 8.8 Hz, 1H), 4.38-4.26 (m, 1H), 4.16 (dd, J = 11.6, 8.7 Hz, 1H), 3.97-3.84 (m, 1H), 3.56-3.44 (m, 4H), 3.31-3.26 (m, 1H), 3.17- 3.07 (m, 1H), 2.72 (d, J = 16.7 Hz, 1H), 2.35-2.31 (m, 1H), 2.17- 1.96 (m, 6H). |
| 165 | 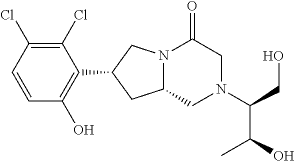 [SMILES: C[C@H](O)[C@H](CO)N1CC(=O)N2C[C@@H](c3c(O)ccc(Cl)c3Cl)C[C@H]2C1] | (7R,8aS)-7-(2,3- dichloro-6- hydroxyphenyl)-2- [(2S,3S)-rel-1,3- dihydroxybutan-2- yl]- hexahydropyrrolo[1, 2-a]pyrazin-4-one | [M + H]⁺: 389, 391 (3:2); ¹H NMR (300 MHz, CD₃OD) δ 7.28 (d, J = 8.8 Hz, 1H), 6.77 (d, J = 8.8 Hz, 1H), 4.55-4.28 (m, 2H), 4.28-4.09 (m, 3H), 4.09-3.91 (m, 3H), 3.83 (dd, J = 13.2, 5.7 Hz, 1H), 3.62 (t, J = 10.6 Hz, 1H), 3.45 (t, J = 11.5 Hz, 1H), 3.29- 3.23 (m, 1H), 2.49-2.43 (m, 1H), 2.34-2.21 (m, 1H), 1.33 (d, J = |
| | | | 6.1 Hz, 3H). |
| 166 | 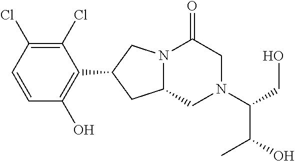 [SMILES: C[C@@H](O)[C@@H](CO)N1CC(=O)N2C[C@@H](c3c(O)ccc(Cl)c3Cl)C[C@H]2C1] | (7R,8aS)-7-(2,3- dichloro-6- hydroxyphenyl)-2- [(2R,3R)-rel-1,3- dihydroxybutan-2- yl]- hexahydropyrrolo[1, 2-a]pyrazin-4-one | [M + H]⁺: 389, 391 (3:2); ¹H NMR (400 MHz, CD₃OD) δ 7.25 (d, J = 8.8 Hz, 1H), 6.75 (d, J = 8.7 Hz, 1H), 4.36-4.25 (m, 1H), 4.16 (dd, J = 11.5, 8.9 Hz, 1H), 3.96-3.86 (m, 1H), 3.86-3.80 (m, 1H), 3.80-3.69 (m, 3H), 3.57- 3.41 (m, 3H), 2.68 (dd, J = 11.9, 9.9 Hz, 1H), 2.53-2.45 (m, 1H), 2.38-2.33 (m, 1H), 2.12-2.06 (m, |
| | | | 1H), 1.23 (d, J = 6.2 Hz, 3H). |
| 167 | 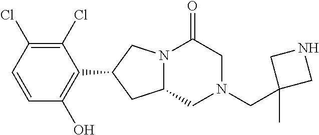 [SMILES: CC1(CN2CC(=O)N3C[C@@H](c4c(O)ccc(Cl)c4Cl)C[C@H]3C2)CNC1] | (7R,8aS)-7-(2,3- dichloro-6- hydroxyphenyl)-2- [(3-methylazetidin- 3-yl)methyl]- hexahydropyrrolo[1, 2-a]pyrazin-4-one | [M + H]⁺: 384, 386 (3:2); ¹H NMR (300 MHz, CD₃OD) δ 7.26 (d, J = 8.8 Hz, 1H), 6.76 (d, J = 8.8 Hz, 1H), 4.40-4.23 (m, 1H), 4.23-4.10 (m, 1H), 4.02 (t, J = 9.9 Hz, 3H), 3.75 (d, J = 10.6 Hz, 2H), 3.59-3.40 (m, 2H), 3.27- 3.09 (m, 2H), 2.70 (s, 2H), 2.56- 2.43 (m, 1H), 2.36-2.31 (m, 1H), |
| | | | 2.18-2.03 (m, 1H), 1.43 (s, 3H). |

### Example 51. Compound 168 ((7R,8aS)-7-(2,3-dichloro-6-hydroxyphenyl)-2-[5-(hydroxymethyl)pyridin-2-yl]-hexahydropyrrolo[1,2-a]pyrazin-4-one)

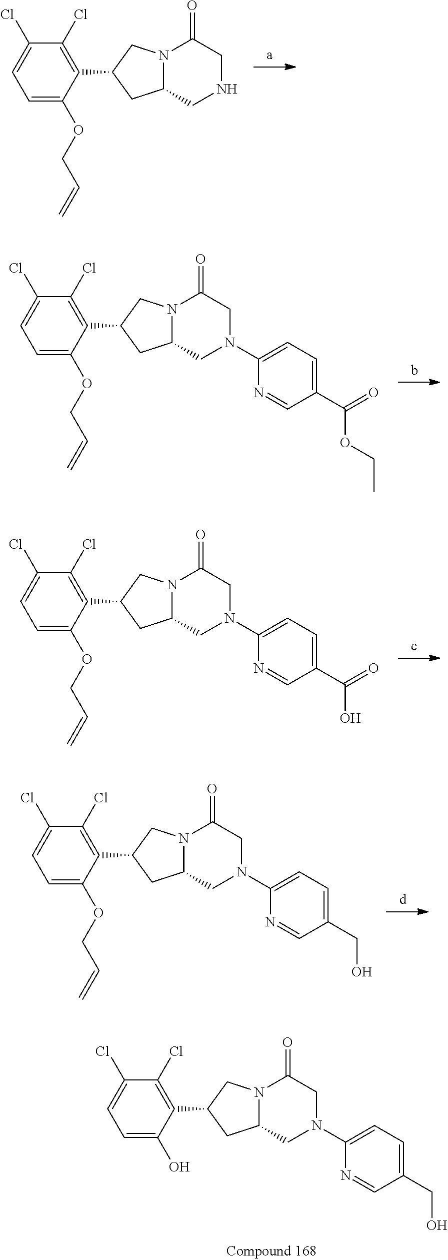 [SMILES: C=CCOc1ccc(Cl)c(Cl)c1[C@H]1C[C@H]2CNCC(=O)N2C1 ; C=CCOc1ccc(Cl)c(Cl)c1[C@H]1C[C@H]2CN(c3ccc(C(=O)OCC)cn3)CC(=O)N2C1 ; C=CCOc1ccc(Cl)c(Cl)c1[C@H]1C[C@H]2CN(c3ccc(C(=O)O)cn3)CC(=O)N2C1 ; C=CCOc1ccc(Cl)c(Cl)c1[C@H]1C[C@H]2CN(c3ccc(CO)cn3)CC(=O)N2C1 ; O=C1CN(c2ccc(CO)cn2)C[C@@H]2C[C@H](c3c(O)ccc(Cl)c3Cl)CN12]

Step a:

A mixture of (7R,8aS)-7-[2,3-dichloro-6-(prop-2-en-1-yloxy)phenyl]-hexahydro-1H-pyrrolo[1,2-a]pyrazin-4-one (Intermediate 13, Example 11) (0.150 g, 0.44 mmol), ethyl 6-chloropyridine-3-carboxylate (0.250 g, 1.32 mmol) and Cs₂CO₃ (0.720 g, 2.20 mmol) in DMSO (1 mL) was stirred at 100° C. for 12 h. The resulting mixture was diluted with water (20 mL) and DCM (20 mL) and extracted with more DCM (3×20 mL). The combined organic layers were washed with brine (2×20 mL) and dried over anhydrous Na₂SO₄. After filtration, the filtrate was concentrated under reduced pressure. The residue was purified by silica gel column chromatography, eluting with EA/PE (1/1) to afford ethyl 6-[(7R,8aS)-7-[2,3-dichloro-6-(prop-2-en-1-yloxy)phenyl]-4-oxo-hexahydropyrrolo[1,2-a]pyrazin-2-yl]pyridine-3-carboxylate as a light yellow oil (0.160 g, 74%): LCMS (ESI) calc'd for C₂₄H₂₅Cl₂N₃O₄ [M+H]⁺: 490, 492 (3:2), found 490, 492 (3:2).

Step b:

A solution of ethyl 6-[(7R,8aS)-7-[2,3-dichloro-6-(prop-2-en-1-yloxy)phenyl]-4-oxo-hexahydropyrrolo[1,2-a]pyrazin-2-yl]pyridine-3-carboxylate (0.170 g, 0.35 mmol) and LiOH H₂O (15 mg, 0.35 mmol) in THF (1 mL), CH₃₀H (0.30 mL) and H₂O (0.30 mL) was stirred at room temperature for 1 h. The mixture was acidified to pH 3 with saturated aq. citric acid (2 mL) and extracted with EA (3×20 mL). The combined organic layers were washed with brine (2×20 mL) and dried over anhydrous Na₂SO₄. After filtration, the filtrate was concentrated under reduced pressure to afford 6-[(7R,8aS)-7-[2,3-dichloro-6-(prop-2-en-1-yloxy)phenyl]-4-oxo-hexahydropyrrolo[1,2-a]pyrazin-2-yl]pyridine-3-carboxylic acid as a light yellow oil (0.160 g, crude), which was used in the next step directly without purification: LCMS (ESI) calc'd for C₂₂H₂₁Cl₂N₃O₄ [M+H]⁺: 462, 464 (3:2), found 462, 464 (3:2).

Step c:

To a stirred solution of 6-[(7R,8aS)-7-[2,3-dichloro-6-(prop-2-en-1-yloxy)phenyl]-4-oxo-hexahydropyrrolo[1,2-a]pyrazin-2-yl]pyridine-3-carboxylic acid (0.160 g, 0.35 mmol) in DME (3 mL) were added 2-methylpropyl chloroformate (95.0 mg, 0.70 mmol) and 4-methylmorpholine (70.0 mg, 0.70 mmol) at 0° C. The reaction was stirred at 0° C. for 1 h under nitrogen atmosphere. Then to the above mixture was added NaBH₄ (26 mg, 0.70 mmol) and the mixture stirred at 0° C. for an additional 1 h. The reaction was quenched with saturated aq. NH₄Cl (20 mL) and extracted with EA (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford (7R,8aS)-7-[2,3-dichloro-6-(prop-2-en-1-yloxy)phenyl]-2-[5-(hydroxymethyl)pyridin-2-yl]-hexahydropyrrolo[1,2-a]pyrazin-4-one as a light yellow oil (0.130 g, 84% overall two steps): LCMS (ESI) calc'd for C₂₂H₂₃Cl₂N₃O₃ [M+H]⁺: 448, 450 (3:2) found 448, 450 (3:2).

Step d:

A mixture of (7R,8aS)-7-[2,3-dichloro-6-(prop-2-en-1-yloxy)phenyl]-2-[5-(hydroxymethyl)pyridin-2-yl]-hexahydropyrrolo[1,2-a]pyrazin-4-one (0.130 g, 0.30 mmol), Pd(PPh₃)₄ (17 mg, 0.01 mmol) and NaBH₄ (22 mg, 0.60 mmol) in THE (2 mL) was stirred at room temperature for 30 min. The resulting mixture was quenched with saturated aq. NH₄Cl (2 mL) and concentrated under reduced pressure. The residue was purified by reverse phase chromatography, eluting with 30% ACN in water (plus 0.05% TFA). The material obtained was further purified by Prep-HPLC with the following conditions: Column: SunFire Prep C18 OBD Column, 5 μm; 19×150 mm; Mobile Phase A: Water (plus 0.05% TFA), Mobile Phase B: ACN; Flow rate: 20 mL/min; Gradient: 5% B to 15% B in 6 min; Detector: UV 210/254 nm; Retention time: 5.85 min. The fractions containing the desired product were collected and concentrated under reduced pressure to afford Compound 168 ((7R,8aS)-7-(2,3-dichloro-6-hydroxyphenyl)-2-[5-(hydroxymethyl)pyridine-2-yl]-hexahydropyrrolo[1,2-a]pyrazin-4-one) as an off-white solid (32.0 mg, 21%): LCMS (ESI) calc'd for C₁₉H₁₉Cl₂N₃O₃ [M+H]⁺: 408, 410 (3:2) found 408, 410 (3:2); ¹H NMR (400 MHz, DMSO-d6) δ 10.50-10.60 (brs, 1H), 8.01 (s, 1H), 7.82-7.77 (m, 1H), 7.36 (dd, J=8.9, 5.8 Hz, 1H), 7.16 (d, J=9.2 Hz, 1H), 6.86 (d, J=8.8 Hz, 1H), 5.30-5.25 (brs, 1H), 4.66 (dd, J=12.7, 3.6 Hz, 1H), 4.47-4.35 (m, 3H), 4.23-4.06 (m, 1H), 4.06-3.89 (m, 2H), 3.85 (d, J=17.1 Hz, 1H), 3.51 (t, J=10.2 Hz, 1H), 3.01 (t, J=11.6 Hz, 1H), 2.35-2.31 (m, 1H), 2.21-2.10 (m, 1H).

### Example 52. Compound 169 ((7R,8aS)-7-(2,3-dichloro-6-hydroxyphenyl)-2-[2-(hydroxymethyl)pyrimidin-4-yl]-hexahydropyrrolo[1,2-a]pyrazin-4-one)

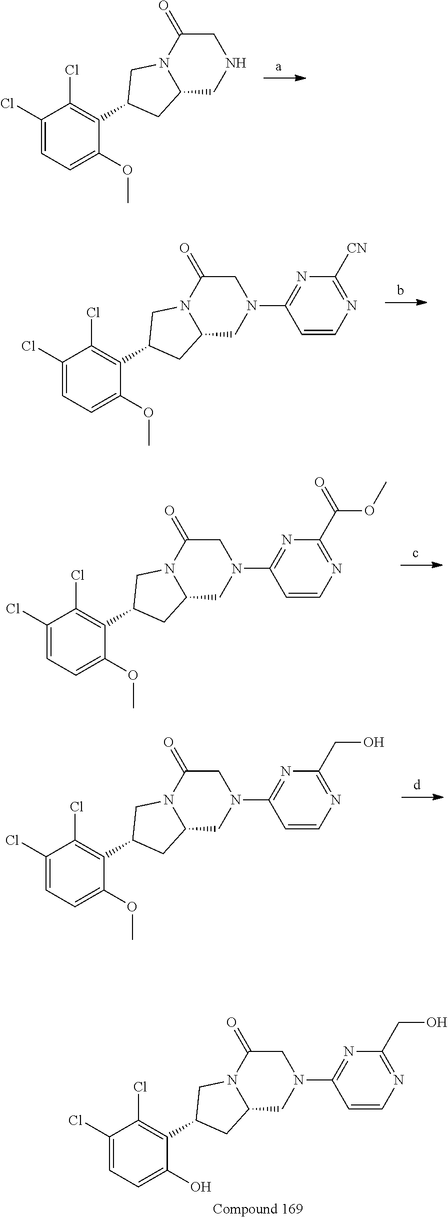 [SMILES: COc1ccc(Cl)c(Cl)c1[C@H]1C[C@H]2CNCC(=O)N2C1 ; COc1ccc(Cl)c(Cl)c1[C@H]1C[C@H]2CN(c3ccnc(C#N)n3)CC(=O)N2C1 ; COC(=O)c1nccc(N2CC(=O)N3C[C@@H](c4c(OC)ccc(Cl)c4Cl)C[C@H]3C2)n1 ; COc1ccc(Cl)c(Cl)c1[C@H]1C[C@H]2CN(c3ccnc(CO)n3)CC(=O)N2C1 ; O=C1CN(c2ccnc(CO)n2)C[C@@H]2C[C@H](c3c(O)ccc(Cl)c3Cl)CN12]

Step a:

To a solution of (7R,8aS)-7-(2,3-dichloro-6-methoxyphenyl)-hexahydro-1H-pyrrolo[1,2-a]pyrazin-4-one (Example 7, step e) (0.180 g, 0.57 mmol) and 4-chloropyrimidine-2-carbonitrile (0.120 g, 0.86 mmol) in DMF (3 mL) was added Cs₂CO₃ (0.370 g, 1.14 mmol) at room temperature. The reaction was stirred at 80° C. for 2 h. After cooling to room temperature the mixture was poured into water (25 mL) and extracted with EA (3×15 mL). The combined organic layers were washed with brine (5×20 mL) and dried over anhydrous Na₂SO₄. After filtration, the filtrate was concentrated under reduced pressure to afford 4-[(7R,8aS)-7-(2,3-dichloro-6-methoxyphenyl)-4-oxo-hexahydropyrrolo[1,2-a]pyrazin-2-yl]pyrimidine-2-carbonitrile as a light yellow solid (0.230 g, crude), which was used in the next step without purification: LCMS (ESI) calc'd for C₁₉H₁₇Cl₂N₅O₂ [M+H]⁺: 418, 420 (3:2) found 418, 420 (3:2); ¹H NMR (400 MHz, CD₃OD) δ 8.31 (d, J=6.4 Hz, 1H), 7.46 (d, J=9.0 Hz, 1H), 7.05-7.01 (m, 2H), 4.68-4.51 (m, 1H), 4.50-4.37 (m, 1H), 4.11 (dd, J=11.6, 8.8 Hz, 1H), 4.07-3.93 (m, 2H), 3.88 (s, 3H), 3.64 (dd, J=11.6, 9.9 Hz, 1H), 3.20-3.03 (m, 2H), 2.42-2.27 (m, 2H).

Step b:

To a solution of 4-[(7R,8aS)-7-(2,3-dichloro-6-methoxyphenyl)-4-oxo-hexahydropyrrolo[1,2-a]pyrazin-2-yl]pyrimidine-2-carbonitrile (0.180 g, 0.43 mmol) in MeOH (4 mL) was added conc. HCl (1 mL) at room temperature. The reaction was stirred at 70° C. for 4 h. After cooling to room temperature, the mixture was concentrated under reduced pressure to remove MeOH. The residue was basified to pH 7 with saturated aq. NaHCO₃ and extracted with EA (3×20 mL). The combined organic layers were washed with brine (2×20 mL) and dried over anhydrous Na₂SO₄. After filtration, the filtrate was concentrated under reduced pressure to afford methyl 4-[(7R,8aS)-7-(2,3-dichloro-6-methoxyphenyl)-4-oxo-hexahydropyrrolo[1,2-a]pyrazin-2-yl]pyrimidine-2-carboxylate as a light yellow solid (0.180 g, 93%): LCMS (ESI) calc'd for C₂₀H₂₀Cl₂N₄O₄ [M+H]⁺: 451, 453 (3:2) found 451, 453 (3:2); ¹H NMR (400 MHz, CD3Cl₃) δ 8.46 (d, J=6.1 Hz, 1H), 7.38 (d, J=8.9 Hz, 1H), 6.80 (d, J=9.0 Hz, 1H), 6.60 (d, J=6.1 Hz, 1H), 4.42-4.25 (m, 2H), 4.19-4.09 (m, 2H), 4.07-4.00 (m, 3H), 4.00-3.90 (m, 2H), 3.85 (s, 3H), 3.69 (dd, J=11.8, 9.3 Hz, 1H), 3.03-2.92 (m, 1H), 2.44-2.21 (m, 2H).

Step c:

To a solution of methyl 4-[(7R,8aS)-7-(2,3-dichloro-6-methoxyphenyl)-4-oxo-hexahydropyrrolo[1,2-a]pyrazin-2-yl]pyrimidine-2-carboxylate (0.180 g, 0.40 mmol) in MeOH (10 mL) was added NaBH₄ (91.0 mg, 2.39 mmol) at room temperature. The reaction was stirred for 1 h, quenched with saturated aq. NH₄Cl (20 mL) and extracted with DCM (3×20 mL). The combined organic layers were washed with brine (2×20 mL) and dried over anhydrous Na₂SO₄. After filtration, the filtrate was concentrated under reduced pressure to afford (7R,8aS)-7-(2,3-dichloro-6-methoxyphenyl)-2-[2-(hydroxymethyl)pyrimidin-4-yl]-hexahydropyrrolo[1,2-a]pyrazin-4-one as a light yellow solid (0.130 g, 77%): LCMS (ESI) calc'd for C₁₉H₂OCl₂N₄O₃ [M+H]⁺: 423, 425 (3:2) found 423, 425 (3:2); ¹H NMR (400 MHz, CDCl₃) δ 8.33 (d, J=9.0 Hz, 1H), 7.39 (d, J=8.7 Hz, 1H), 6.81 (d, J=8.7 Hz, 1H), 6.42 (d, J=9.0 Hz, 1H), 5.12-4.97 (m, 1H), 4.67 (s, 2H), 4.47-4.30 (m, 2H), 4.17 (t, J=10.5 Hz, 1H), 4.06-3.90 (m, 2H), 3.86 (s, 3H), 3.74-3.65 (m, 1H), 2.97 (t, J=11.2 Hz, 1H), 2.42-2.21 (m, 2H).

Step d:

To a solution of (7R,8aS)-7-(2,3-dichloro-6-methoxyphenyl)-2-[2-(hydroxymethyl)pyrimidin-4-yl]-hexahydropyrrolo[1,2-a]pyrazin-4-one (0.130 g, 0.31 mmol) in DCM (4 mL) was added BBr₃ (0.50 mL, 5.29 mmol) at room temperature. The reaction was stirred at room temperature for 2 h then quenched with MeOH (5 mL) at 0° C. and concentrated under reduced pressure. The residue was purified by Prep-HPLC with the following conditions: Column: Sunfire prep C₁₈ column, 30×150, 5 μm; Mobile Phase A: Water (with 10 mmol/L NH₄HCO₃), Mobile Phase B: ACN; Flow rate: 20 mL/min; Gradient: 20% B to 70% B in 9 min; Detector: UV 210 nm; Retention Time: 8.6 min. The fractions containing the desired product were collected and concentrated under reduced pressure to afford Compound 169 ((7R,8aS)-7-(2,3-dichloro-6-hydroxyphenyl)-2-[2-(hydroxymethyl)pyrimidin-4-yl]-hexahydropyrrolo[1,2-a]pyrazin-4-one) as an off-white solid (36.6 mg, 29%): LCMS (ESI) calc'd for C₁₈H₁₈Cl₂N₄O₃ [M+H]⁺: 409, 411 (3:2) found 409, 411 (3:2); ¹H NMR (400 MHz, DMSO-d₆) δ 10.55-10.45 (brs, 1H), 8.24 (d, J=6.1 Hz, 1H), 7.36 (d, J=8.8 Hz, 1H), 6.86 (d, J=8.8 Hz, 1H), 6.75 (d, J=6.2 Hz, 1H), 4.95-4.90 (brs, 2H), 4.54 (s, 1H), 4.40 (s, 2H), 4.25-4.11 (m, 1H), 4.06-4.01 (m, 1H), 3.92-3.85 (m, 1H), 3.82 (d, J=17.5 Hz, 1H), 3.48 (t, J=10.2 Hz, 1H), 2.96-2.90 (m, 1H), 2.35-2.30 (m, 1H), 2.22-2.11 (m, 1H).

Example 53. Compounds 170-174 were prepared in an analogous fashion as that described for Compound 169.

| Compound | | | |
|---|---|---|---|
| Number | Structure | Chemical Name | MS: (M + H)⁺ & ¹H MNR |
| 170 | 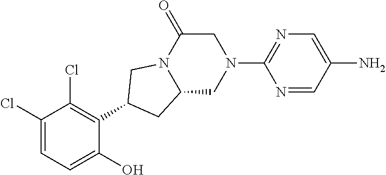 [SMILES: Nc1cnc(N2CC(=O)N3C[C@@H](c4c(O)ccc(Cl)c4Cl)C[C@H]3C2)nc1] | (7R,8aS)-2-(5- aminopyrimidin- 2-yl)-7-(2,3- dichloro-6- hydroxyphenyl)- hexahydropyrrolo [1,2-a]pyrazin-4- one | [M + H]⁺: 394, 396 (3:2); ¹H NMR (400 MHz, CD₃OD) δ 8.31 (s, 2H), 7.28 (d, J = 8.8 Hz, 1H), 6.78 (d, J = 8.8 Hz, 1H), 5.10 (dd, J = 13.2, 3.7 Hz, 1H), 4.82 (d, J = 18.4 Hz, 1H), 4.44-4.28 (m, 1H), 4.28-4.18 (m, 1H), 4.04-3.96 (m, 1H), 3.91 (d, J = 18.4 Hz, 1H), 3.65- 3.58 (m, 1H), 3.03 (dd, J = 13.1, 10.4 Hz, 1H), 2.53-2.49 |
| | | | (m, 1H), 2.28-2.18 (m, 1H). |
| 171 | 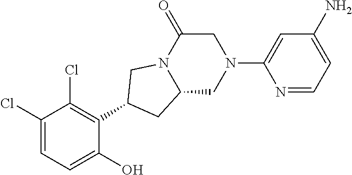 [SMILES: Nc1ccnc(N2CC(=O)N3C[C@@H](c4c(O)ccc(Cl)c4Cl)C[C@H]3C2)c1] | (7R,8aS)-2-(4- aminopyridin-2- yl)-7-(2,3- dichloro-6- hydroxyphenyl)- hexahydropyrrolo [1,2-a]pyrazin-4- one | [M + H]⁺: 393, 395 (3:2); ¹H NMR (400 MHz, CD₃OD) δ 7.65 (d, J = 6.3 Hz, 1H), 7.28 (d, J = 8.8 Hz, 1H), 6.78 (d, J = 8.8 Hz, 1H), 6.21 (dd, J = 6.3, 1.9 Hz, 1H), 6.01 (d, J = 2.0 Hz, 1H), 4.51 (dd, J = 12.8, 3.6 Hz, 1H), 4.43-4.26 (m, 2H), 4.27-4.16 (m, 1H), 4.07-3.98 (m, 1H), 3.81 (d, J = 17.2 Hz, 1H), 3.68-3.60 (m, 1H), 2.98 |
| | | | (dd, J = 12.7, 10.4 Hz, 1H), |
| | | | 2.53-2.49 (m, 1H), 2.30-2.19 |
| | | | (m, 1H). |
| 172 | 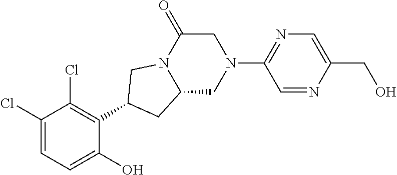 [SMILES: O=C1CN(c2cnc(CO)cn2)C[C@@H]2C[C@H](c3c(O)ccc(Cl)c3Cl)CN12] | (7R,8aS)-1-(2,3- dichloro-6- hydroxyphenyl)- 2-[5- (hydroxymethyl) pyrazin-2-yl]- hexahydropyrrolo [1,2-a]pyrazin-4- one | [M + H]⁺: 409, 411 (3:2); ¹H NMR (400 MHz, DMSO-d₆) δ 10.55-10.45 (brs, 1H), 8.25 (d, J = 1.5 Hz, 1H), 8.15 (d, J = 1.4 Hz, 1H), 7.36 (d, J = 8.8 Hz, 1H), 6.85 (d, J = 8.8 Hz, 1H), 5.30-5.25 (brs, 1H), 4.79 (dd, J = 12.8, 3.7 Hz, 1H), 4.54-4.42 (m, 3H), 4.23-4.10 (m, 1H), 4.10-3.97 (m, 1H), 3.97-3.83 (m, 1H), 3.78 (d, J = |
| | | | 17.4 Hz, 1H), 3.50-3.46 (m, |
| | | | 1H), 2.89 (dd, J = 12.9, 10.4 |
| | | | Hz, 1H), 2.35-2.30 (m, 1H), |
| | | | 2.21-2.10 (m, 1H). |
| 173 | 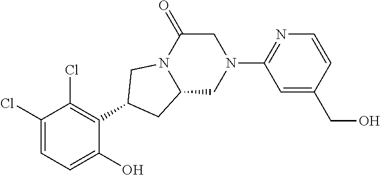 [SMILES: O=C1CN(c2cc(CO)ccn2)C[C@@H]2C[C@H](c3c(O)ccc(Cl)c3Cl)CN12] | (7R,8aS)-1-(2,3- dichloro-6- hydroxyphenyl)- 2-[4- (hydroxymethyl) pyridin-2-yl]- hexahydropyrrolo [1,2-a]pyrazin-4- one | [M + H]⁺: 408, 410 (3:2); ¹H NMR (400 MHz, DMSO-d₆) δ 10.39 (s, 1H), 8.06 (d, J = 5.1 Hz, 1H), 7.36 (d, J = 8.8 Hz, 1H), 6.84 (d, J = 8.8 Hz, 1H), 6.77 (s, 1H), 6.66 (dd, J = 5.1, 1.1 Hz, 1H), 5.31 (t, J = 5.8 Hz, 1H), 4.78 (dd, J = 12.7, 3.7 Hz, 1H), 4.47 (d, J = 5.6 Hz, 2H), 4.39 (d, J = 17.4 Hz, 1H), 4.22-4.08 (m, 1H), 4.07-3.98 |
| | | | (m, 1H), 3.96-3.82 (m, 1H), |
| | | | 3.69 (d, J = 17.5 Hz, 1H), 3.46 |
| | | | (t, J = 10.3 Hz, 1H), 2.82 (dd, J = |
| | | | 12.9, 10.4 Hz, 1H), 2.36-2.23 |
| | | | (m, 1H), 2.18-2.11 (m, 1H). |
| 174 | 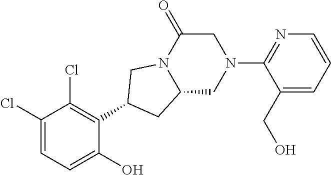 [SMILES: O=C1CN(c2ncccc2CO)C[C@@H]2C[C@H](c3c(O)ccc(Cl)c3Cl)CN12] | (7R,8aS)-7-(2,3- dichloro-6- hydroxyphenyl)-2- [3- (hydroxymethyl) pyridin-2-yl]- hexahydropyrrolo 1,2-a]pyrazin-4- one trifluoroacetic acid | [M + H]⁺: 408, 410 (3:2); ¹H NMR (400 MHz, CD₃OD) δ 8.21 (dd, J = 5.1, 1.9 Hz, 1H), 7.97 (dd, J = 7.5, 1.9 Hz, 1H), 7.26 (d, J = 8.8 Hz, 1H), 7.16 (dd, J = 7.5, 5.1 Hz, 1H), 6.77 (d, J = 8.8 Hz, 1H), 4.70 (s, 2H), 4.40-4.28 (m, 1H), 4.28- 4.18 (m, 1H), 4.18-4.00 (m, 3H), 3.95 (d, J = 17.3 Hz, 1H), 3.60 (t, J = 10.5 Hz, 1H), 3.09 |
| | | | (dd, J = 13.0, 10.1 Hz, 1H), |
| | | | 2.45-2.36 (m, 1H), 2.20-2.12 |
| | | | (m, 1H). |

### Example 54. Compound 175 ((7R,8aS)-7-(2,3-dichloro-6-hydroxyphenyl)-2-(1H-1,2,4-triazol-5-yl)hexahydropyrrolo[1,2-a]pyrazin-4(1H)-one)

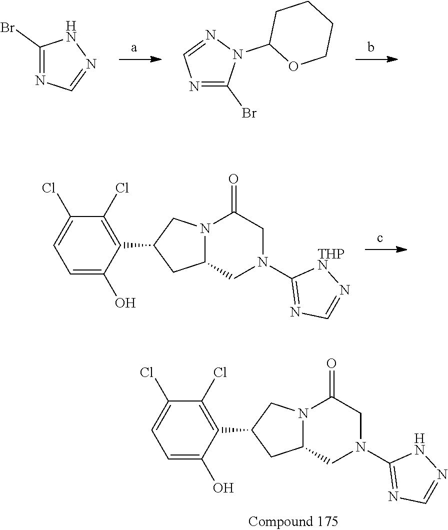

Step a:

To a stirred solution of 3-bromo-2H-1,2,4-triazole (0.500 g, 3.38 mmol) and DHP (0.310 g, 3.72 mmol) in THF (5 mL) was added TsOH (58.0 mg, 0.34 mmol) at room temperature. The reaction was stirred at 50° C. for 2 h. The resulting mixture was diluted with EA (30 mL) and saturated aq. Na₂CO₃ (30 mL) and extracted with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 50% ACN in water (plus 10 mM NH₄HCO₃) to afford 5-bromo-1-(tetrahydro-2H-pyran-2-yl)-1,2,4-triazole as a light yellow oil (0.400 g, 51%): LCMS (ESI) calc'd for C₇H₁₀BrN₃O [M+H]⁺: 232 found 232; ¹H NMR (400 MHz, CDCl₃) δ 8.18 (s, 1H), 5.45 (dd, J=8.7, 3.0 Hz, 1H), 4.13-4.03 (m, 1H), 3.77-3.65 (m, 1H), 2.24-2.13 (m, 1H), 2.13-1.97 (m, 2H), 1.82-1.62 (m, 3H).

Step b:

To a stirred solution of (7R,8aS)-7-(2,3-dichloro-6-hydroxyphenyl)-hexahydro-1H-pyrrolo[1,2-a]pyrazin-4-one (50.0 mg, 0.17 mmol,) and 5-bromo-1-(tetrahydro-2H-pyran-2yl)-1,2,4-triazole (58.0 mg, 0.25 mmol) in dioxane (1 mL) were added Pd₂(dba)₃ (15.0 mg, 0.02 mmol), XantPhos (10 mg, 0.02 mmol) and Cs₂CO₃ (0.160 g, 0.49 mmol) at room temperature under nitrogen atmosphere. The resulting mixture was stirred at 100° C. for 16 h. After cooling to room temperature, the mixture was diluted with water (10 mL) and extracted with EA (3×10 mL). The combined organic layers were washed with brine (3×10 mL) and dried over anhydrous Na₂SO₄. After filtration, the filtrate was concentrated under reduced pressure. The residue was purified by reverse flash chromatography eluting with 45% ACN in water (plus 0.05% TFA) to afford (7R,8aS)-7-(2,3-dichloro-6-hydroxyphenyl)-2-[2-(tetrahydro-2H-pyran-2yl)-1,2,4-triazol-3-yl]-hexahydropyrrolo[1,2-a]pyrazin-4-one as a brown oil (60 mg, 0, 68%): LCMS (ESI) calc'd for C₂₀H₂₃Cl₂N₅O₃ [M+H]⁺: 452, 454 (3:2) found 452, 454 (3:2); ¹H NMR (400 MHz, CDCl₃) δ 8.21 (s, 1H), 7.19 (d, J=8.8 Hz, 1H), 6.86 (d, J=8.8 Hz, 1H), 5.31 (dd, J=9.0, 2.9 Hz, 1H), 4.53 (d, J=18.1 Hz, 1H), 4.48-4.33 (m, 2H), 4.23-4.09 (m, 2H), 3.94 (d, J=18.3 Hz, 2H), 3.73 (t, J=10.9 Hz, 1H), 3.52-3.41 (m, 1H), 3.07 (dd, J=12.9, 10.3 Hz, 1H), 2.35-2.31 (m, 1H), 2.25-2.16 (m, 1H), 2.15-2.00 (m, 3H), 1.77-1.62 (m, 3H).

Step c:

To a stirred solution of (7R,8aS)-7-(2,3-dichloro-6-hydroxyphenyl)-2-[2-(tetrahydro-2H-pyran-2yl)-1,2,4-triazol-3-yl]-hexahydropyrrolo[1,2-a]pyrazin-4-one (30.0 mg, 0.06 mmol) in dioxane (0.5 mL) was added HCl (6 N, 0.25 mL) at room temperature. The resulting mixture was stirred for 0.5 h and concentrated under reduce pressure. The residue was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 15% to 40% in 7 min; Detector: UV 254/220 nm; Retention time: 7.32 min. The fractions containing the desired product were collected and concentrated under reduced pressure to afford Compound 175 ((7R,8aS)-7-(2,3-dichloro-6-hydroxyphenyl)-2-(2H-1,2,4-triazol-3-yl)-hexahydropyrrolo[1,2-a]pyrazin-4-one) as an off-white solid (25.5 mg, 37.87%): LCMS (ESI) calc'd C₁₅H₁₅Cl₂N₅O₂ for [M+H]⁺: 368, 370 (3:2) found 368, 370 (3:2); ¹H NMR (400 MHz, CD₃OD) δ 8.15 (s, 1H), 7.28 (d, J=8.8 Hz, 1H), 6.78 (d, J=8.8 Hz, 1H), 4.41-4.30 (m, 3H), 4.26-4.15 (m, 1H), 4.13-4.00 (m, 1H), 3.91 (d, J=17.3 Hz, 1H), 3.63 (dd, J=11.5, 9.6 Hz, 1H), 3.21-3.10 (m, 1H), 2.50-2.47 (m, 1H), 2.30-2.17 (m, 1H).

### Example 55. Compound 176 ((7R,8aS)-7-(2,3-dichloro-6-hydroxyphenyl)-2-(1H-imidazol-2-yl)-hexahydropyrrolo[1,2-a]pyrazin-4-one)

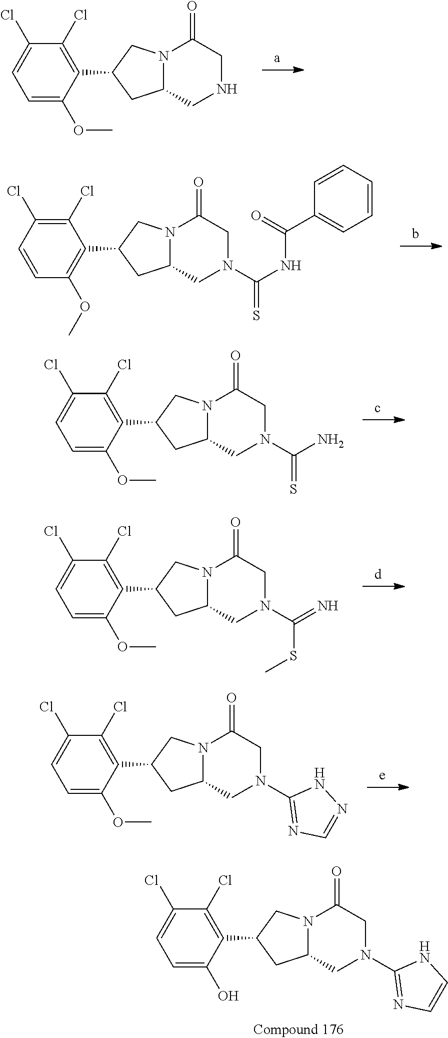

Step a:

To a stirred solution of (7R,8aS)-7-(2,3-dichloro-6-methoxyphenyl)-hexahydro-1H-pyrrolo[1,2-a]pyrazin-4-one (Example 7, step e) (0.300 g, 0.95 mmol) in MeCN (4 mL) was added benzoyl isothiocyanate (0.190 g, 1.14 mmol) at room temperature. The reaction was stirred for 1 h, diluted with water (30 mL) and extracted with EA (3×30 mL). The combined organic layers were washed with brine (3×30 mL) and dried over anhydrous Na₂SO₄. After filtration, the filtrate was concentrated under reduced pressure to afford N-[(7R,8aS)-7-(2,3-dichloro-6-methoxyphenyl)-4-oxo-hexahydropyrrolo[1,2-a]pyrazine-2-carbothioyl]benzamide as a light yellow solid (0.500 g, crude), which was used in the next step without purification: LCMS (ESI) calc'd for C₂₂H₂₁Cl₂N₃O₃S [M+H]⁺: 478, 480 (3:2) found 478, 480 (3:2); ¹H NMR (300 MHz, CDCl₃) δ 8.72-7.99 (m, 1H), 7.93-7.78 (m, 2H), 7.71-7.59 (m, 1H), 7.59-7.45 (m, 2H), 7.37 (d, J=8.8 Hz, 1H), 6.79 (d, J=9.0 Hz, 1H), 5.44-4.98 (m, 1H), 4.54-4.21 (m, 3H), 4.14 (t, J=10.4 Hz, 2H), 3.85 (s, 3H), 3.73-3.58 (m, 1H), 3.32 (dd, J=13.1, 10.6 Hz, 1H), 2.41-2.08 (m, 1H), 1.83-1.62 (m, 1H).

Step b:

A solution of N-[(7R,8aS)-7-(2,3-dichloro-6-methoxyphenyl)-4-oxo-hexahydropyrrolo[1,2-a]pyrazine-2-carbothioyl]benzamide (0.500 g, 1.05 mmol) in hydrazine (5 mL) was stirred at room temperature for 1 h. The precipitated solid were collected by filtration and washed with MeOH (3×5 mL). The solid was dried under vacuum to afford (7R,8aS)-7-(2,3-dichloro-6-methoxyphenyl)-4-oxo-hexahydropyrrolo[1,2-a]pyrazine-2-carbothioamide as a grey solid (0.260 g, 73% over two steps): LCMS (ESI) calc'd for C₁₅H₁₇Cl₂N₃O₂S [M+H]⁺: 374, 376 (3:2) found 374, 376 (3:2); ¹H NMR (300 MHz, DMSO-d₆) δ 7.63 (s, 2H), 7.58-7.52 (m, 1H), 7.09 (d, J=9.1 Hz, 1H), 5.17-4.96 (m, 1H), 4.70-4.52 (m, 1H), 4.27-4.11 (m, 1H), 4.02-3.86 (m, 3H), 3.83 (s, 3H), 3.52-3.41 (m, 1H), 2.98 (t, J=11.7 Hz, 1H), 2.22-2.03 (m, 2H).

Step c:

To a stirred solution of (7R,8aS)-7-(2,3-dichloro-6-methoxyphenyl)-4-oxo-hexahydropyrrolo[1,2-a]pyrazine-2-carbothioamide (0.240 g, 0.64 mmol) in THE (4 mL) was added Mel (0.270 g, 1.92 mmol) at room temperature. The reaction was stirred at room temperature overnight and concentrated under reduced pressure. The residue was purified by reverse phase chromatography, eluting with 40% ACN in water (plus 0.05% TFA) to afford (7R,8aS)-7-(2,3-dichloro-6-methoxyphenyl)-2-methylsulfanylcarboximidoyl-hexahydropyrrolo[1,2-a]pyrazin-4-one as a light yellow oil (0.150 g, 51%): LCMS (ESI) calc'd for C₁₆H₁₉Cl₂N₃O₂S [M+H]⁺: 388, 390 (3:2) found 388, 390 (3:2); ¹H NMR (300 MHz, CD₃OD) δ 7.45 (d, J=9.0 Hz, 1H), 7.02 (d, J=9.1 Hz, 1H), 4.64-4.36 (m, 3H), 4.25-3.99 (m, 3H), 3.87 (s, 3H), 3.73-3.61 (m, 1H), 3.44-3.34 (m, 1H), 2.75 (s, 3H), 2.37-2.26 (m, 2H).

Step d:

To a stirred solution of (7R,8aS)-7-(2,3-dichloro-6-methoxyphenyl)-2-methylsulfanylcarboximidoyl-hexahydropyrrolo[1,2-a]pyrazin-4-one (0.150 g, 0.39 mmol) in pyridine (3 mL) was added 2,2-dimethoxyethanamine (81.0 mg, 0.77 mmol) at room temperature. The reaction was stirred at 110° C. for 2 h. After cooling to room temperature, the mixture was concentrated under reduced pressure. HCl (2 N, 2 mL) was added over 1 min at room temperature and the resulting mixture was stirred at 90° C. for 30 min. The resulting mixture was diluted with water (10 mL), basified to pH 8 with saturated aq. NaHCO₃ and extracted with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 30% ACN in water (plus 0.05% TFA) to afford (7R,8aS)-7-(2,3-dichloro-6-methoxyphenyl)-2-(1H-imidazol-2-yl)-hexahydropyrrolo[1,2-a]pyrazin-4-one as a light yellow solid (0.130 g, 75%); LCMS (ESI) calc'd for C₁₇H₁₈Cl₂N₄O₂ [M+H]⁺: 381, 383 (3:2) found 381, 383 (3:2); ¹H NMR (300 MHz, CD₃OD) δ 7.45 (d, J=9.0 Hz, 1H), 7.06-6.99 (m, 3H), 4.47-4.35 (m, 1H), 4.32-4.15 (m, 2H), 4.15-4.02 (m, 3H), 3.87 (s, 3H), 3.71-3.60 (m, 1H), 3.39-3.34 (m, 1H), 2.39-2.26 (m, 2H).

Step e:

To a stirred solution of (7R,8aS)-7-(2,3-dichloro-6-methoxyphenyl)-2-(1H-imidazol-2-yl)-hexahydropyrrolo[1,2-a]pyrazin-4-one (0.130 g, 0.34 mmol) in DCM (2 mL) was added BBr₃ (0.5 mL, 5.29 mmol) dropwise at room temperature. The reaction was stirred for 2 h, quenched with water (5 mL) and concentrated under reduced pressure. The residue was purified by Prep-HPLC with the following conditions: Column: XSelect CSH Prep C18 OBD Column, 19×250 mm, 5 μm; Mobile Phase A: Water (plus 0.05% TFA), Mobile Phase B: ACN; Flow rate: 20 mL/min; Gradient: 28% B to 40% B in 5.3 min; Detector: UV 254/210 nm; Retention time: 5.36 min. The fractions containing the desired product were collected and concentrated under reduced pressure to afford Compound 176 ((7R,8aS)-7-(2,3-dichloro-6-hydroxyphenyl)-2-(1H-imidazol-2-yl)-hexahydropyrrolo[1,2-a]pyrazin-4-one) as an off-white solid (49.0 mg, 29%); LCMS (ESI) calc'd for C₁₆H₁₆Cl₂N₄O₂ [M+H]⁺: 367, 369 (3:2) found 367, 369 (3:2); ¹H NMR (400 MHz, CD₃OD) δ 7.28 (d, J=8.8 Hz, 1H), 6.87 (s, 2H), 6.78 (d, J=8.8 Hz, 1H), 4.42-4.31 (m, 1H), 4.29-4.15 (m, 3H), 4.13-4.03 (m, 1H), 3.91 (d, J=16.8 Hz, 1H), 3.68-3.60 (m, 1H), 3.20-3.12 (m, 1H), 2.52-2.49 (m, 1H), 2.27-2.20 (m, 1H).

### Example 56. Compound 43 ((3S,8R,9aS)-8-(2,3-dichloro-6-hydroxyphenyl)-3-(hydroxymethyl)hexahydropyrido[2,1-c][1,4]oxazin-4(3H)-one) and Compound 52 ((3R,8R,9aS)-8-(2,3-dichloro-6-hydroxyphenyl)-3-(hydroxymethyl)hexahydropyrido[2,1-c][1,4]oxazin-4(3B)-one)

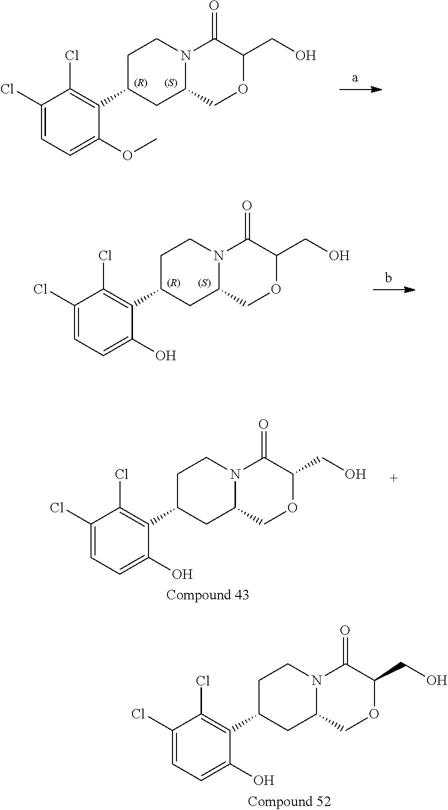

Step a:

To a stirred solution of (8R,9aS)-8-(2,3-dichloro-6-methoxyphenyl)-3-(hydroxymethyl)-hexahydro-1H-pyrido[2,1-c][1,4]oxazin-4-one (66.0 mg, 0.18 mmol) in DCM (2 mL) was added BBr₃ (0.25 mL, 2.64 mmol) at room temperature. The reaction was stirred at room temperature for 1 h, quenched with MeOH (1 mL) and concentrated under reduced pressure. The residue was purified by Prep-HPLC with the following conditions Column: Xselect CSH OBD Column 30×150 mm 5 μm; Mobile Phase A: Water (plus 0.05% TFA), Mobile Phase B: ACN; Flow rate: 60 mL/min; Gradient: 25% to 45% in 7 min; Detector: UV 254/220 nm; Retention time: 6.67 min. The fractions containing the desired product were collected and concentrated under reduced pressure to afford (8R,9aS)-8-(2,3-dichloro-6-hydroxyphenyl)-3-(hydroxymethyl)-hexahydro-1H-pyrido[2,1-c][1,4]oxazin-4-one as an off-white solid (20 mg, 31%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.20 (dd, J=8.8, 2.8 Hz, 1H), 6.71 (d, J=8.5 Hz, 1H), 4.78-4.64 (m, 1H), 4.22-4.10 (m, 1H), 4.04-3.85 (m, 4H), 3.84-3.66 (m, 1H), 3.66-3.47 (m, 1H), 2.82-0.67 (m, 2H), 2.57-2.20 (m, 1H), 1.71-1.52 (m, 2H).

Step b:

(8R,9aS)-8-(2,3-dichloro-6-hydroxyphenyl)-3-(hydroxymethyl)-hexahydro-1H-pyrido[2,1-c][1,4]oxazin-4-one was separated by Prep Chiral-HPLC with the following conditions: Column: CHIRALPAK IC, 2×25 cm, 5 μm; Mobile Phase A: Hex (plus 0.2% IPA)-HPLC, Mobile Phase B: EtOH-HPLC; Flow rate: 20 mL/min; Gradient: 15% B to 15% B in 11.5 min; Detector: UV 254/220 nm; Retention time 1: 9.49 min; Retention time 2: 10.77 min. The faster-eluting enantiomer at 9.49 min was obtained Compound 43 ((3S,8R,9aS)-8-(2,3-dichloro-6-hydroxyphenyl)-3-(hydroxymethyl)-hexahydro-1H-pyrido[2,1-c][1,4]oxazin-4-one) as an off-white solid (22 mg, 31%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.20 (d, J=8.8 Hz, 1H), 6.71 (d, J=8.8 Hz, 1H), 4.74-4.66 (m, 1H), 4.15 (t, J=4.5 Hz, 1H), 4.01 (dd, J=12.2, 4.0 Hz, 1H), 3.93 (d, J=4.4 Hz, 2H), 3.89 (dd, J=12.2, 2.8 Hz, 1H), 3.83-3.71 (m, 1H), 3.53-3.48 (m, 1H), 2.82-2.71 (m, 2H), 2.54-2.40 (m, 1H), 1.66-1.55 (m, 2H). And the slower-eluting enantiomer at 10.77 min was obtained Compound 52 ((3R,8R,9aS)-8-(2,3-dichloro-6-hydroxyphenyl)-3-(hydroxymethyl)-hexahydro-1H-pyrido[2,1-c][1,4]oxazin-4-one) as an off-white solid (8 mg, 11%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.20 (d, J=8.8 Hz, 1H), 6.71 (d, J=8.8 Hz, 1H), 4.77-4.69 (m, 1H), 4.20-4.12 (m, 2H), 4.00-3.85 (m, 2H), 3.78-3.64 (m, 2H), 3.60 (dd, J=11.9, 9.4 Hz, 1H), 2.79-2.67 (m, 1H), 2.55-2.41 (m, 1H), 2.30-2.26 (m, 1H), 1.66-1.64 (m, 2H).

Example 57. Compound 50 was prepared in an analogous fashion as that described for Compounds 43 and 52.

| Compound | | | |
|---|---|---|---|
| Number | Structure | Chemical Name | MS: (M + H)⁺ & ¹H MNR |
| 50 | 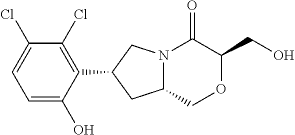 [SMILES: O=C1[C@@H](CO)OC[C@@H]2C[C@H](c3c(O)ccc(Cl)c3Cl)CN12] | (3R,7R,8aS)-7-(2,3- dichloro-6- hydroxyphenyl)-3- (hydroxymethyl)- hexahydropyrrolo[2,1- c][1,4]oxazin-4-one | [M + H]⁺: 332, 334 (3:2); ¹H NMR (400 MHz, CD₃OD) δ 7.26 (d, J = 8.8 Hz, 1H), 6.76 (d, J = 8.8 Hz, 1H), 4.38-4.25 (m, 2H), 4.21 (dd, J = 11.4, 9.3 Hz, 1H), 4.14 (t, J = 3.4 Hz, 1H), 4.09-3.99 (m, 1H), 3.98-3.86 (m, 2H), 3.61-3.49 (m, 2H), 2.35-2.30 (m, 1H), |
| | | | 2.14-2.05 (m, 1H). |

### Example 58. Compound 180 ((8R,9aS)-3-(aminomethyl)-8-(2,3-dichloro-6-hydroxyphenyl)-hexahydro-1H-pyrido[2,1-c][1,4]oxazin-4-one)

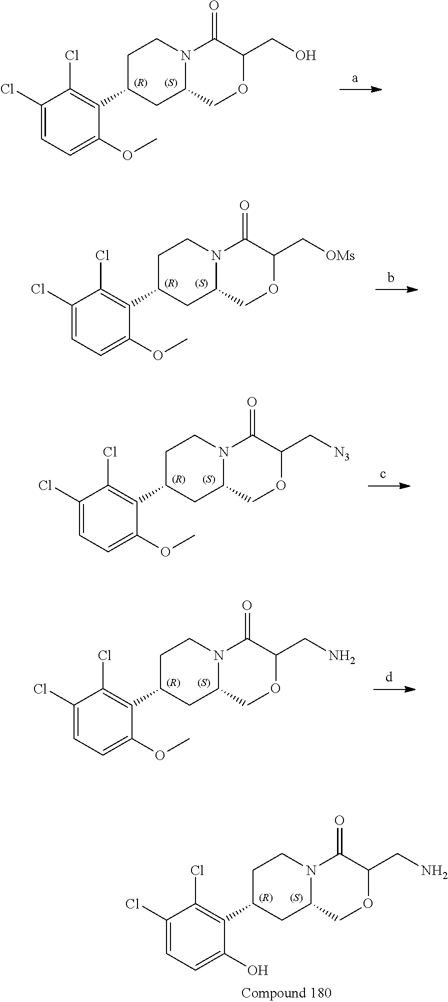

Step a:

To a stirred solution of (8R,9aS)-8-(2,3-dichloro-6-methoxyphenyl)-3-(hydroxymethyl)-hexahydro-1H-pyrido[2,1-c][1,4]oxazin-4-one (Intermediate 14, Example 12) (0.580 g, 1.61 mmol) and TEA (0.325 g, 3.22 mmol) in DCM (2.00 mL, 31.5 mmol) was added Ms-Cl (0.276 g, 2.42 mmol) at 0° C. The reaction was stirred at 0° C. for 1 h under nitrogen atmosphere. The resulting mixture was quenched with saturated aq. NH₄Cl (20 mL) at 0° C. and extracted with DCM (3×10 mL). The combined organic layers were washed with brine (3×10 mL) and dried over anhydrous MgSO₄. After filtration, the filtrate was concentrated under reduced pressure to afford [(8R,9aS)-8-(2,3-dichloro-6-methoxyphenyl)-4-oxo-hexahydro-1H-pyrido[2,1-c][1,4]oxazin-3-yl]methyl methanesulfonate as a light yellow oil (0.780 g, crude), which was used directly in the next step without purification: LCMS (ESI) calc'd for C₁₆H₂OCl₂N₂O₃ [M+H]⁺: 438, 440 (3:2) found 438, 440 (3:2).

Step b:

To a stirred solution of [(8R,9aS)-8-(2,3-dichloro-6-methoxyphenyl)-4-oxo-hexahydro-1H-pyrido[2,1-c][1,4]oxazin-3-yl]methyl methanesulfonate (0.540 g, 1.23 mmol) in DMF (8 mL) was added NaN₃ (0.160 g, 2.46 mmol) at room temperature under nitrogen atmosphere. The reaction was stirred at 80° C. for 12 h. The cooled mixture was quenched with saturated aq. NaHCO₃ (30 mL) and extracted with EA (3×20 mL). The combined organic layers were washed with brine (3×10 mL) and dried over anhydrous Na₂SO₄. After filtration, the filtrate was used directly in the next step. LCMS (ESI) calc'd for C₁₆H₁₈Cl₂N₄O₃ [M+H]⁺: 385, 387 (3:2), found 385, 387 (3:2).

Step c:

To a stirred solution of (8R,9aS)-3-(azidomethyl)-8-(2,3-dichloro-6-methoxyphenyl)-hexahydro-1H-pyrido[2,1-c][1,4]oxazin-4-one (0.480 g, 1.23 mmol) in EtOAc (10 mL) was added PtO₂ (50.0 mg, 0.22 mmol) at room temperature under nitrogen atmosphere. The suspension was degassed under reduced pressure and purged with hydrogen three times. The mixture was stirred under hydrogen atmosphere (1.5 atm) at room temperature for 2 h, filtered and concentrated under reduced pressure to afford (8R,9aS)-3-(aminomethyl)-8-(2,3-dichloro-6-methoxyphenyl)-hexahydro-1H-pyrido[2,1-c][1,4]oxazin-4-one as a light yellow oil (0.410 g, crude), which was used in the next step without purification: LCMS (ESI) calc'd for C₁₆H₂₀Cl₂N₂O₃ [M+H]⁺: 359, 361 (3:2) found 359, 361 (3:2).

Step d:

To a stirred solution of (8R,9aS)-3-(aminomethyl)-8-(2,3-dichloro-6-methoxyphenyl)-hexahydro-1H-pyrido[2,1-c][1,4]oxazin-4-one (30.0 mg, 0.08 mmol) in DCM (2 mL) was added BBr₃ (0.10 mL, 1.06 mmol) at room temperature under nitrogen atmosphere. The reaction was stirred for 2 h, quenched with MeOH (2 mL) and concentrated under reduced pressure. The residue was purified by Prep-HPLC with the following conditions: Column: XBridge Shield RP18 OBD Column, 30×150 mm, 5 μm; Mobile Phase A: Water (plus 10 mM NH₄HCO₃), Mobile Phase B: ACN; Flow rate: 60 mL/min; Gradient: 25% B to 50% B in 8 min; Detector: UV 220 nm; Retention time: 5.92 min. The fractions containing the desired product were collected and concentrated under reduced pressure to afford Compound 180 ((8R,9aS)-3-(aminomethyl)-8-(2,3-dichloro-6-hydroxyphenyl)-hexahydro-1H-pyrido[2,1-c][1,4]oxazin-4-one) as an off-white solid (10.4 mg, 36%): LCMS (ESI) calc'd for C₁₅H₁₈Cl₂N₂O₃ [M+H]⁺: 345, 347 (3:2) found 345, 347 (3:2); ¹H NMR (400 MHz, CD₃OD) δ 7.20 (d, J=8.8 Hz, 1H), 6.71 (d, J=8.7 Hz, 1H), 4.71 (t, J=15.5 Hz, 1H), 4.21-3.98 (m, 2H), 3.88-3.47 (m, 3H), 3.13-3.02 (m, 2H), 2.88-2.19 (m, 3H), 1.71-1.54 (m, 2H).

### Example 59. Compound 181 (8R,9aS)-8-(2,3-dichloro-6-hydroxyphenyl)-3-(pyrrolidin-1-ylmethyl)-hexahydro-1H-pyrido[2,1-c][1,4]oxazin-4-one

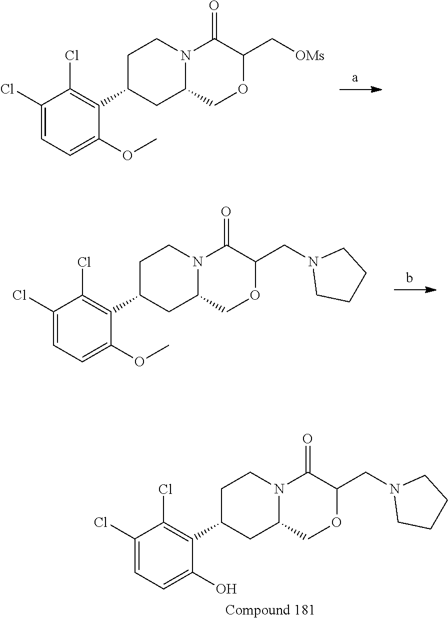

Step a:

To a stirred mixture of [(8R,9aS)-8-(2,3-dichloro-6-methoxyphenyl)-4-oxo-hexahydro-1H-pyrido[2,1-c][1,4]oxazin-3-yl]methyl methanesulfonate (Example 58, step a) (40.0 mg, 0.09 mmol) and pyrrolidine (32.0 mg, 0.46 mmol) in ACN (1 mL) was added DIEA (35.0 mg, 0.27 mmol) at room temperature. The reaction mixture was stirred at 80° C. for 3 h. The cooled mixture was quenched with water (20 mL) and extracted with EA (3×30 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 0.05% TFA) to afford (8R,9aS)-8-(2,3-dichloro-6-methoxyphenyl)-3-(pyrrolidin-1-ylmethyl)-hexahydro-1H-pyrido[2,1-c][1,4]oxazin-4-one as a yellow oil (20 mg, 48%): LCMS (ESI) calc'd for C₂₀H₂₆Cl₂N₂O₃ [M+H]⁺: 413, 415 (3:2) found 413, 415 (3:2).

Step b:

To a stirred mixture of (8R,9aS)-8-(2,3-dichloro-6-methoxyphenyl)-3-(pyrrolidin-1-ylmethyl)-hexahydro-1H-pyrido[2,1-c][1,4]oxazin-4-one (20.0 mg, 0.05 mmol) in DCM (1 mL) was added BBr₃ (0.25 mL, 2.6 mmol) at room temperature. The reaction was stirred at room temperature for 1 h, quenched with MeOH (2 mL) and concentrated under reduced pressure. The residue was purified by Prep-HPLC with the following conditions: Column: Xselect CSH OBD Column 30×150 mm 5 μm, n; Mobile Phase A: Water (plus 0.05% TFA), Mobile Phase B: ACN; Flow rate: 60 mL/min; Gradient: 15% B to 40% B in 7 min; Detector: UV 220 nm; Retention time: 6.32 min. The fractions containing the desired product were collected and concentrated under reduced pressure to afford Compound 182 ((8R,9aS)-8-(2,3-dichloro-6-hydroxyphenyl)-3-(pyrrolidin-1-ylmethyl)-hexahydro-1H-pyrido[2,1-c][1,4]oxazin-4-one) as a white solid (4.8 mg, 24.35%): LCMS (ESI) calc'd for C₁₉H₂₄Cl₂N₂O₃ [M+H]⁺: 399, 401 (3:2) found 399, 401 (3:2); ¹H NMR (400 MHz, CD₃OD) δ 7.22 (d, J=8.8 Hz, 1H), 6.72 (d, J=8.8 Hz, 1H), 4.75-4.67 (m, 1H), 4.63-4.52 (m, 1H), 4.27-4.12 (m, 1H), 4.00-3.56 (m, 7H), 3.24-3.13 (m, 2H), 2.86-2.72 (m, 1H), 2.58-2.41 (m, 1H), 2.33-2.30 (m, 1H), 2.24-2.13 (m, 2H), 2.12-2.00 (m, 2H), 1.73-1.63 (m, 2H).

Example 60. Compounds 182-183 were prepared in an analogous fashion to an example disclosed herein and/or analogous to known methods in the art.

| Compound | | | MS: (M + H)⁺ & ¹H |
|---|---|---|---|
| Number | Structure | Chemical Name | MNR |
| 182 | 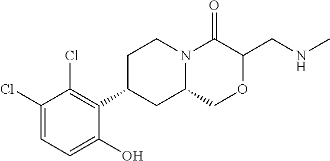 | (8R,9aS)-8-(2,3- dichloro-6- hydroxyphenyl)- 3- [(methylamino) methyl]-hexahydro- 1H-pyrido[2,1- c][1,4]oxazin-4- one | [M + H]⁺: 359, 361 (3: 2); ¹H NMR (400 MHz, CD₃OD) δ 7.21 (dd, J = 8.8, 1.2 Hz, 1H), 6.73 (dd, J = 8.8, 5.3 Hz, 1H), 4.76-4.65 (m, 1H), 4.53-4.43 (m, 1H), 4.25- 4.08 (m, 1H), 3.97-3.56 (m, 3H), 3.56-3.47 (m, 1H), 3.47-3.37 (m, 1H), |
| | | | 2.86-2.69 (m, 4.5H), |
| | | | 2.58-2.37 (m, 1H), 2.31 |
| | | | (q, J = 11.2 Hz, 0.5H), |
| | | | 1.73-1.57 (m, 2H). |
| 183 | 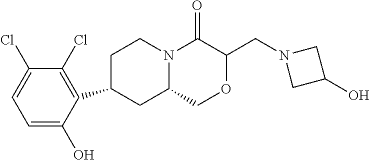 | (8R,9aS)-8-(2,3- dichloro-6- hydroxyphenyl)-3- [(3- hydroxyazetidin- 1-yl)methyl]- hexahydro-1H- pyrido[2,1- c][1,4]oxazin-4- one | [M + H]+: 401, 403 (3: 2); ¹H NMR (400 MHz, CD₃OD) δ 7.22 (d, J = 8.8 Hz, 1H), 6.73 (d, J = 8.8 Hz, 1H), 4.75-4.60 (m, 2H), 4.52-4.36 (m, 3H), 4.26-3.86 (m, 4H), 3.88-3.54 (m, 4H), 2.89- 2.66 (m, 1H), 2.56-2.35 (m, 1H), 2.32-2.29 (m, |
| | | | 1H), 1.73-1.58 (m, 2H). |

### Example 61. Compound 184 (1-[[(8R,9aS)-8-(2,3-dichloro-6-hydroxyphenyl)-4-oxo-hexahydro-1H-pyrido[2,1-c][1,4]oxazin-3-yl]methyl]pyrrolidin-2-one)

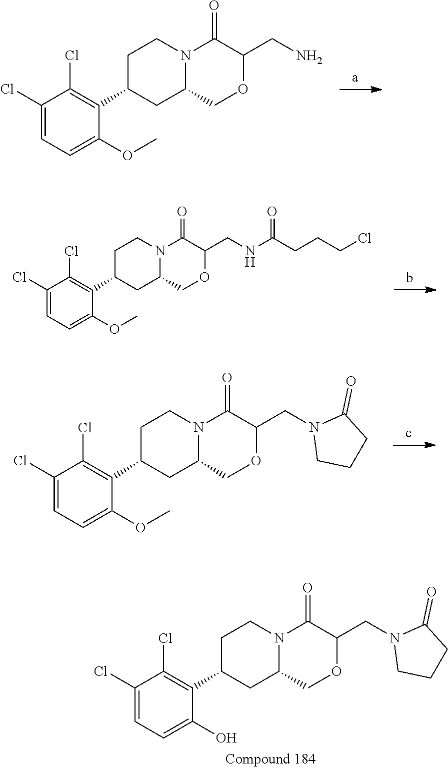

Step a:

To a stirred solution of (8R,9aS)-3-(aminomethyl)-8-(2,3-dichloro-6-methoxyphenyl)-hexahydro-1H-pyrido[2,1-c][1,4]oxazin-4-one (Example 58, step c) (60.0 mg, 0.17 mmol) and TEA (34.0 mg, 0.33 mmol) in DCM (1 mL) was added 4-chlorobutanoyl chloride (28.0 mg, 0.20 mmol) dropwise at 0° C. The reaction was stirred at room temperature for 1 h, diluted with water (20 mL) and extracted with EA (3×20 mL). The combined organic layers were washed with brine (2×20 mL) and dried over anhydrous Na₂SO₄. After filtration, the filtrate was concentrated under reduced pressure to afford N-[[(8R,9aS)-8-(2,3-dichloro-6-methoxyphenyl)-4-oxo-hexahydro-1H-pyrido[2,1-c][1,4]oxazin-3-yl]methyl]-4-chlorobutanamide as a yellow oil (0.100 g, crude), which was used in the next step without purification: LCMS (ESI) calc'd for C₂₀H₂₅C₁₃N₂O₄ [M+H]⁺: 463, 465, 467 (3:3:1), found 463, 465, 467 (3:3:1).

Step b:

To a stirred solution of N-[[(8R,9aS)-8-(2,3-dichloro-6-methoxyphenyl)-4-oxo-hexahydro-1H-pyrido[2,1-c][1,4]oxazin-3-yl]methyl]-4-chlorobutanamide (90.0 mg, 0.19 mmol) in DMF (1 mL) was added Cs₂CO₃ (0.130 mg, 0.39 mmol) at room temperature. The reaction was stirred for 12 h, diluted with water (20 mL) and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 30% ACN in water (plus 0.5% TFA). The fractions containing the desired product were collected and concentrated under reduced pressure to afford 1-[[(8R,9aS)-8-(2,3-dichloro-6-methoxyphenyl)-4-oxo-hexahydro-1H-pyrido[2,1-c][1,4]oxazin-3-yl]methyl]pyrrolidin-2-one as a yellow oil (35.0 mg, 38%): LCMS (ESI) calc'd for C₂₀H₂₄Cl₂N₂O₄ [M+H]⁺: 427, 429 (3:2) found 427, 429 (3:2); ¹H NMR (400 MHz, CDCl₃) δ 7.33 (d, J=8.9 Hz, 1H), 6.77 (d, J=8.9 Hz, 1H), 4.76 (d, J=13.2 Hz, 1H), 4.55-4.44 (m, 1H), 4.16-4.09 (m, 1H), 3.91-3.78 (m, 5H), 3.75-3.48 (m, 5H), 2.85-2.68 (m, 1H), 2.65-2.61 (m, 2H), 2.44-2.27 (m, 2H), 2.23-2.13 (m, 2H), 1.80-1.58 (m, 2H).

Step c:

To a stirred mixture of 1-[[(8R,9aS)-8-(2,3-dichloro-6-methoxyphenyl)-4-oxo-hexahydro-1H-pyrido[2,1-c][1,4]oxazin-3-yl]methyl]pyrrolidin-2-one (35.0 mg, 0.08 mmol) in DCM (1 mL) was added BBr₃ (0.25 mL, 2.64 mmol) at 0° C. The reaction was stirred at room temperature for 1 h then quenched with MeOH (2 mL) at 0° C. and concentrated under reduced pressure. The residue was purified by Prep-HPLC with the following conditions: Column: XBridge Prep OBD C18 Column, 30×150 mm 5 μm; Mobile Phase A: Water (plus 10 mM NH₄HCO₃), Mobile Phase B: ACN; Flow rate: 60 mL/min; Gradient: 22% B to 53% B in 8 min; Detector: UV 254/220 nm; Retention time: 7.57 min. The fractions containing the desired product were collected and concentrated under reduced pressure to afford Compound 184 (1-[[(8R,9aS)-8-(2,3-dichloro-6-hydroxyphenyl)-4-oxo-hexahydro-1H-pyrido[2,1-c][1,4]oxazin-3-yl]methyl]pyrrolidin-2-one) as an white solid (10.3 mg, 29.5%): LCMS (ESI) calc'd for C₁₉H₂₂Cl₂N₂O₄ [M+H]⁺: 413, 415 (3:2) found 413, 415 (3:2); ¹H NMR (400 MHz, CD₃OD) δ 7.21 (d, J=8.8 Hz, 1H), 6.73 (d, J=8.8 Hz, 1H), 4.79-4.66 (m, 1H), 4.40-4.27 (m, 1H), 4.17-3.93 (m, 1H), 3.91-3.81 (m, 2H), 3.81-3.63 (m, 2H), 3.62-3.48 (m, 3H), 2.83-2.64 (m, 2H), 2.52-2.27 (m, 3H), 2.15-2.01 (m, 2H), 1.68-1.58 (m, 2H).

Example 62. Compounds 185-186 were prepared in an analogous fashion as that described for Compound 184.

| Compound | | | MS: (M + H)⁺ & ¹H |
|---|---|---|---|
| Number | Structure | Chemical Name | MNR |
| 185 | 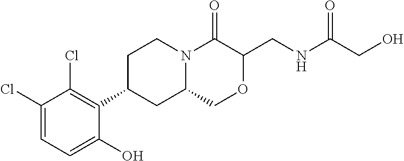 | N-[[(8R,9aS)-8-(2,3- dichloro-6- hydroxyphenyl)-4-oxo- hexahydro-1H-pyrido[2,1- c][1,4]oxazin-3- yl]methyl]-2- hydroxyacetamide | [M + H]⁺: 403, 405 (3:2); ¹H NMR (400 MHz, CD₃OD) δ 7.20 (d, J = 8.8 Hz, 1H), 6.72 (d, J = 8.8 Hz, 1H), 4.75-4.65 (m, 1H), 4.31-3.94 (m, 4H), 3.91-3.47 (m, 5H), 2.84- 2.23 (m, 3H), 1.66-1.56 (m, 2H). |
| 186 | 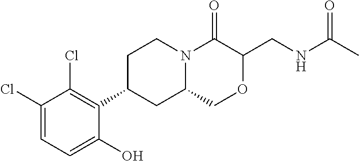 | N-[[(8R,9aS)-8-(2,3- dichloro-6- hydroxyphenyl)-4-oxo- hexahydro-1H-pyrido[2,1- c][1,4]oxazin-3- yl]methyl]acetamide | [M + H]⁺: 387, 389 (3:2); ¹H NMR (400 MHz, CD₃OD) δ 7.20 (d, J = 8.8 Hz, 1H), 6.72 (d, J = 8.8 Hz, 1H), 4.73-4.68 (m, 1H), 4.25-3.95 (m, 2H), 3.87-3.65 (m, 3H), 3.62- 3.45 (m, 2H), 2.83-2.23 (m, 3H), 1.98 (d, J = 9.3 Hz, 3H), 1.66-1.57 (m, |
| | | | 2H). |

### Example 63. Compound 187 (2-[(8R,9aS)-8-(2,3-dichloro-6-hydroxyphenyl)-4-oxo-hexahydro-1H-pyrido[2,1-c][1,4]oxazin-3-yl]acetamide)

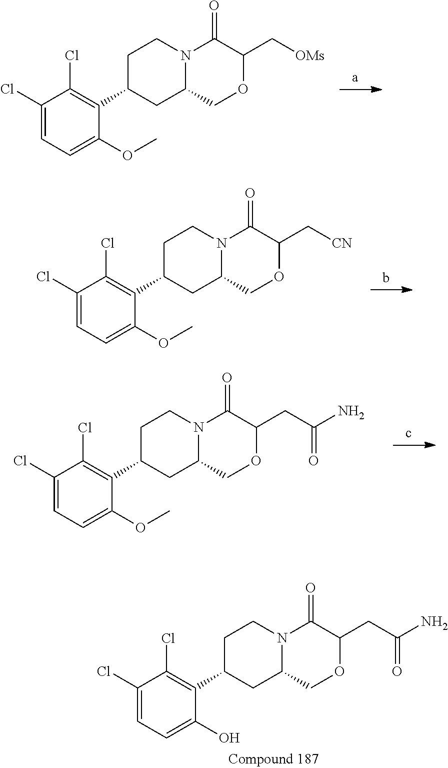

Step a:

To a stirred mixture of [(8R,9aS)-8-(2,3-dichloro-6-methoxyphenyl)-4-oxo-hexahydro-1H-pyrido[2,1-c][1,4]oxazin-3-yl]methyl methanesulfonate (Example 58, step a) (0.600 g, 1.37 mmol) in DMF (8 mL) was added NaCN (0.200 g, 4.11 mmol) at room temperature. The reaction mixture was stirred at 80° C. for 16 h under nitrogen atmosphere. The resulting mixture was quenched with saturated aqueous NaHCO₃ (20 mL) at room temperature and extracted with EA (3×50 mL). The combined organic layers were washed with brine (3×20 mL) and dried over anhydrous Na₂SO₄. After filtration, the filtrate was concentrated under reduced pressure. The residue was purified by reverse phase chromatography, eluting with 40% ACN in water with 10 mM NH₄HCO₃ to afford 2-[(8R,9aS)-8-(2,3-dichloro-6-methoxyphenyl)-4-oxo-hexahydro-1H-pyrido[2,1-c][1,4]oxazin-3-yl]acetonitrile as a yellow solid (0.150 g, 25%): LCMS (ESI) calc'd for C₁₇H₁₈Cl₂N₂O₃ [M+H]⁺: 369, 371 (3:2) found 369, 371 (3:2); ¹H NMR (400 MHz, CDCl₃) δ 7.34-7.30 (m, 1H), 6.78-6.74 (m, 1H), 4.85-4.73 (m, 1H), 4.42-4.30 (m, 1H), 4.19-4.02 (m, 1H), 3.91-3.72 (m, 4H), 3.72-3.53 (m, 1H), 3.15-3.04 (m, 1H), 2.96-2.86 (m, 1H), 2.81-2.67 (m, 1H), 2.47-2.29 (m, 1H), 2.11-1.98 (m, 1H), 1.74-1.59 (m, 3H).

Step b:

To a stirred mixture of 2-[(8R,9aS)-8-(2,3-dichloro-6-methoxyphenyl)-4-oxo-hexahydro-1H-pyrido[2,1-c][1,4]oxazin-3-yl]acetonitrile (30.0 mg, 0.08 mmol) and NaOH (32.0 mg, 0.81 mmol) in MeOH (1 mL) was added H₂O₂ (23.0 mg, 0.81 mmol) at room temperature. The reaction was stirred at room temperature for 1 h, quenched with saturated aq. Na₂S₂O₃ (15 mL) at 0° C. and extracted with EA (3×15 mL). The combined organic layers were washed with brine (3×20 mL) and dried over anhydrous Na₂SO₄. After filtration, the filtrate was concentrated under reduced pressure to afford 2-[(8R,9aS)-8-(2,3-dichloro-6-methoxyphenyl)-4-oxo-hexahydro-1H-pyrido[2,1-c][1,4]oxazin-3-yl]acetamide as an off-white solid (20.0 mg, 51%): LCMS (ESI) calc'd for C₁₇H₂₀Cl₂N₂O₄ [M+H]⁺: 387, 389 (3:2) found 387, 389 (3:2); ¹H NMR (400 MHz, CD₃OD) δ 7.39 (d, J=9.0 Hz, 1H), 6.97 (d, J=9.0 Hz, 1H), 4.75-4.56 (m, 1H), 4.53-4.44 (m, 1H), 4.31 (t, J=6.6 Hz, 1H), 4.16-3.97 (m, 1H), 3.86 (d, J=8.4 Hz, 3H), 3.83-3.63 (m, 1H), 3.63-3.49 (m, 1H), 2.95-2.81 (m, 1H), 2.80-2.61 (m, 2H), 2.40-2.30 (m, 1H), 2.22-2.10 (m, 1H), 1.73-1.54 (m, 2H).

Step c:

To a stirred mixture of 2-[(8R,9aS)-8-(2,3-dichloro-6-methoxyphenyl)-4-oxo-hexahydro-1H-pyrido[2,1-c][1,4]oxazin-3-yl]acetamide (30.0 mg, 0.07 mmol) in DCM (1 mL) was added BBr₃ (97.0 mg, 0.38 mmol) at room temperature. The reaction was stirred for 1 h., quenched with saturated aq. NaHCO₃ (2 mL) and concentrated under reduced pressure. The residue was purified by Prep-HPLC with the following conditions: Column: XBridge Shield RP18 OBD Column, 30×150 mm, 5 μm; Mobile Phase A: Water (plus 0.05% TFA), Mobile Phase B: ACN; Flow rate: 60 mL/min; Gradient: 20% B to 50% B in 7 min; Detector: UV 220 nm; Retention time: 6.4 min. The fractions containing the desired product were collected and concentrated under reduced pressure to afford Compound 187 (2-[(8R,9aS)-8-(2,3-dichloro-6-hydroxyphenyl)-4-oxo-hexahydro-1H-pyrido[2,1-c][1,4]oxazin-3-yl]acetamide) as an off-white solid (7.3 mg, 24%): LCMS (ESI) calc'd for C₁₆H₁₈Cl₂N₂O₄ [M+H]⁺: 373, 375 (3:2) found 373, 375 (3:2); ¹H NMR (400 MHz, CD₃OD) δ 7.23 (d, J=8.7 Hz, 1H), 6.72 (d, J=8.7 Hz, 1H), 4.75-4.65 (m, 1H), 4.55-4.46 (m, 1H), 4.09 (dd, J=11.9, 4.5 Hz, 1H), 3.83-3.64 (m, 2H), 3.59 (dd, J=12.0, 9.7 Hz, 1H), 2.87 (dd, J=15.4, 3.7 Hz, 1H), 2.78-2.61 (m, 2H), 2.55-2.43 (m, 1H), 2.30-2.28 (m, 1H), 1.70-1.58 (m, 2H).

Example 64. Compound 188 was prepared in an analogous fashion as that described for Compound 187.

| Compound | | | |
|---|---|---|---|
| Number | Structure | Chemical Name | MS: (M + H)⁺ & ¹H MNR |
| 188 | 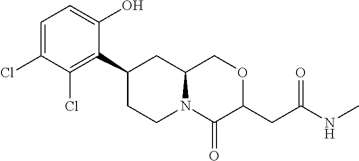 | 2-[(8R,9aS)-8-(2,3- dichloro-6- hydroxyphenyl)-4- oxo-hexahydro-1H- pyrido[2,1-c] [1,4]oxazin-3-yl]-N- methylacetamide | [M + H]⁺: 387, 389 (3:2); ¹H NMR (400 MHz, CD₃OD) δ 7.20 (d, J = 8.8 Hz, 1H), 6.72 (d, J = 8.8 Hz, 1H), 4.74-4.65 (m, 1H), 4.55-4.45 (m, 1H), 4.13-3.94 (m, 1H), 3.84-3.62 (m, 2H), 3.58 (dd, J = 11.9, 9.7 Hz, 1H), 2.92-2.69 (m, 5H), 2.66-2.56 (m, 1H), 2.56-2.40 (m, 1H), 2.31-2.27 (m, 1H), |
| | | | 1.72-1.55 (m, 2H). |

### Example 65. Compound 189 ((2R, 8aS)-7-amino-2-(2,3-dichloro-6-hydroxyphenyl)hexahydroindolizin-5(1H)-one isomer 1) and Compound 190 ((2R,8aS)-7-amino-2-(2,3-dichloro-6-hydroxyphenyl)hexahydroindolizin-5(1H)-one isomer 2)

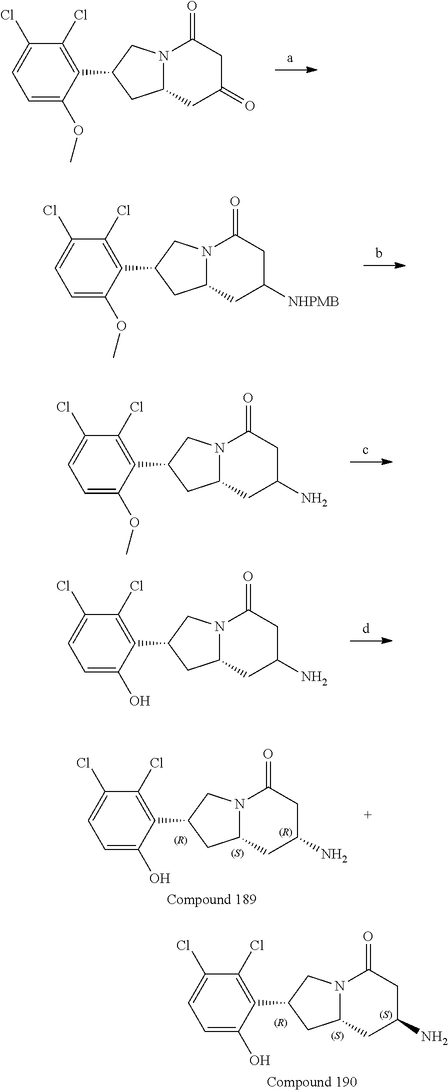

Step a.

To a stirred mixture of (2R,8aS)-2-(2,3-dichloro-6-methoxyphenyl)-hexahydroindolizine-5,7-dione (Intermediate 15, Example 13) (80.0 mg, 0.24 mmol) and 4-methoxy-benzylamine (50.0 mg, 0.37 mmol) in DCM (3 mL) were added AcOH (14.0 mg, 0.24 mmol) and NaBH(OAc)₃ (0.150 g, 0.73 mmol) in portions at room temperature. The reaction was stirred for 1 h and NaBH₄ (18.0 mg, 0.49 mmol) was then added. The reaction was stirred for additional 2 h at room temperature, and quenched with saturated aq. NH₄Cl (20 mL) followed by extraction with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 0.1% FA) to afford (2R,8aS)-2-(2,3-dichloro-6-methoxyphenyl)-7-[[(4-methoxyphenyl)methyl]amino]-hexahydro-1H-indolizin-5-one as a light yellow semisolid (60.0 mg, 55%): LCMS (ESI) calc'd for C₂₃H₂₆Cl₂N₂O₃ [M+H]⁺ 449, 451 (3:2) found 449, 451 (3:2); ¹H NMR (400 MHz, CD₃OD) δ 7.49-7.34 (m, 3H), 7.06-6.98 (m, 3H), 5.51 (s, 2H), 4.41-4.30 (m, 1H), 4.26-4.11 (m, 2H), 4.07-3.96 (m, 1H), 3.91-3.76 (m, 7H), 3.76-3.61 (m, 1H), 3.58-3.48 (m, 1H), 2.98 (dd, J=17.2, 6.4 Hz, 1H), 2.72-2.62 (m, 1H), 2.50-2.38 (m, 1H), 2.34-2.19 (m, 1H), 1.67-1.55 (m, 1H).

Step b:

To a stirred solution of (2R,8aS)-2-(2,3-dichloro-6-methoxyphenyl)-7-[[(4-methoxyphenyl)methyl]amino]-hexahydro-1H-indolizin-5-one (60.0 mg, 0.13 mmol) in MeCN (2 mL) and H₂O (0.5 mL) was added Ce(NO₃)₄.2NH₄NO₃ (0.150 g, 0.27 mmol) at room temperature. The reaction was stirred for 16 h, and quenched with saturated aq. Na₂SO₃ (20 mL) followed by extraction with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 45% ACN in water (plus 0.1% FA) to afford (2R,8aS)-7-amino-2-(2,3-dichloro-6-methoxyphenyl)-hexahydro-1H-indolizin-5-one as a yellow oil (40.0 mg, 91%): LCMS (ESI) calc'd for C₁₅H₁₈Cl₂N₂O₂ [M+H]⁺ 329, 331 (3:2) found 329, 331 (3:2); ¹H NMR (400 MHz, CD₃OD) δ 7.44 (d, J=9.0 Hz, 1H), 7.02 (d, J=8.9 Hz, 1H), 4.42-4.31 (m, 1H), 4.11-3.88 (m, 2H), 3.85 (s, 3H), 3.79-3.67 (m, 1H), 3.54 (t, J=11.0 Hz, 1H), 2.92-2.80 (m, 1H), 2.57-2.36 (m, 2H), 2.35-2.21 (m, 2H), 1.71-1.57 (m, 1H).

Step c:

To a stirred solution of (2R,8aS)-7-amino-2-(2,3-dichloro-6-methoxyphenyl)-hexahydro-1H-indolizin-5-one (40.0 mg, 0.12 mmol) in DCM (2 mL) was added BBr₃ (0.300 g, 1.22 mmol) at room temperature. The reaction was stirred for 1 h, quenched with MeOH (1 mL) and concentrated under reduced pressure. The residue was purified by reverse phase chromatography, eluting with 30% ACN in water (plus 0.1% FA) to afford the crude product. The crude was purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 15% B to 35% B in 5.5 min; Detector: UV 210 nm; Retention time: 5.6 min. The fractions containing the desired product were collected and concentrated under vacuum to afford (2R,8aS)-7-amino-2-(2,3-dichloro-6-hydroxyphenyl)-hexahydro-1H-indolizin-5-one as an off-white solid (12.2 mg, 27.8%): LCMS (ESI) calc'd for C₁₄H₁₆Cl₂N₂O₂ [M+H]⁺ 315, 317 (3:2) found 315, 317 (3:2); ¹H NMR (400 MHz, CD₃OD) δ 8.51 (s, 1H), 7.26 (d, J=8.8 Hz, 1H), 6.77 (d, J=8.8 Hz, 1H), 4.36-4.24 (m, 1H), 4.12 (dd, J=11.6, 9.2 Hz, 1H), 3.88-3.77 (m, 1H), 3.75-3.61 (m, 1H), 3.54 (dd, J=11.7, 9.9 Hz, 1H), 2.89-2.80 (m, 1H), 2.54-2.32 (m, 3H), 2.27-2.17 (m, 1H), 1.65-1.62 (m, 1H).

Step d:

The product (2R,8aS)-7-amino-2-(2,3-dichloro-6-hydroxyphenyl)-hexahydro-1H-indolizin-5-one (10.0 mg, 0.03 mmol) was separated by Prep Chiral HPLC with the following conditions: Column: CHIRALPAK IE, 2×25 cm, 5 μm; Mobile Phase A: Hex/DCM=3/1 (10 mM NH₃-MeOH)-HPLC, Mobile Phase B: EtOH-HPLC; Flow rate: 20 mL/min; Gradient: 20% to 20% in 11 min; Detector: UV 220/254 nm; Retention time 1: 7.49 min; Retention time 2: 8.65 min. The faster-eluting isomer at 7.49 min gave Compound 189 ((2R, 8aS)-7-amino-2-(2,3-dichloro-6-hydroxyphenyl)-hexahydro-1H-indolizin-5-one isomer 1) as an off-white solid (4.00 mg, 47.6%): LCMS (ESI) calc'd for C₁₄H₁₆Cl₂N₂O₂ [M+H]⁺: 315, 317 (3:2) found 315, 317 (3:2); ¹H NMR (400 MHz, CD₃OD) δ 7.26 (d, J=8.8 Hz, 1H), 6.79 (d, J=8.9 Hz, 1H), 4.30-4.09 (m, 2H), 3.88-3.49 (m, 3H), 2.89-2.74 (m, 1H), 2.56-2.40 (m, 2H), 2.36 (dd, J=17.1, 10.7 Hz, 1H), 2.27-2.02 (m, 1H), 1.68-1.48 (m, 1H). The slower-eluting isomer at 8.65 min gave Compound 190 ((2R, 8aS)-7-amino-2-(2,3-dichloro-6-hydroxyphenyl)-hexahydro-1H-indolizin-5-one isomer 2) as an off-white solid (4.50 mg, 53.6%): LCMS (ESI) calc'd for C₁₄H₁₆Cl₂N₂O₂ [M+H]⁺: 315, 317 (3:2) found 315, 317 (3:2); ¹H NMR (400 MHz, CD₃OD) δ 7.25 (d, J=8.8 Hz, 1H), 6.75 (d, J=8.7 Hz, 1H), 4.34-4.16 (m, 1H), 4.16-4.03 (m, 1H), 3.83-3.72 (m, 1H), 3.50 (dd, J=11.6, 9.9 Hz, 1H), 3.31-3.23 (m, 1H), 2.71 (dd, J=17.5, 6.0 Hz, 1H), 2.45-2.27 (m, 2H), 2.23-2.09 (m, 2H), 1.50-1.40 (m, 1H).

### Example 66. Compound 191 ((2R, 8aS)-2-(2,3-dichloro-6-hydroxyphenyl)-7-[(3R)-3-hydroxypyrrolidin-1-yl]-hexahydro-1H-indolizin-5-one isomer 1) and Compound 192 ((2R,8aS)-2-(2,3-dichloro-6-hydroxyphenyl)-7-[(3R)-3-hydroxypyrrolidin-1-yl]-hexahydro-1H-indolizin-5-one isomer 2)

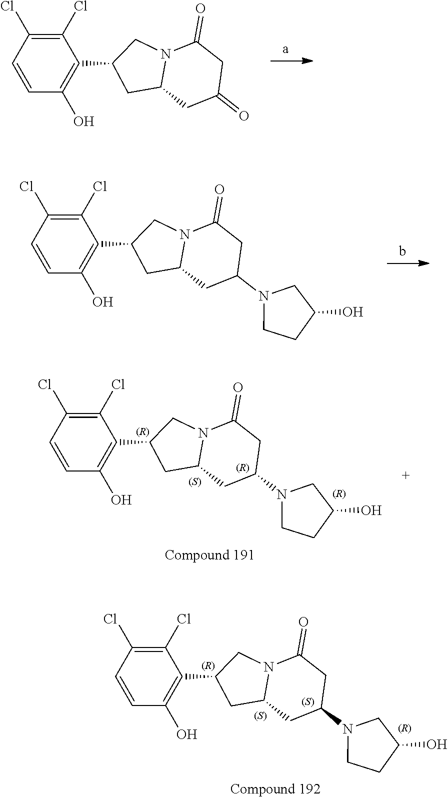

Step a:

A mixture of (2R,8aS)-2-(2,3-dichloro-6-hydroxyphenyl)-hexahydroindolizine-5,7-dione (27.0 mg, 0.09 mmol) and (3R)-pyrrolidin-3-ol hydrochloride (0.150 g, 1.20 mmol) in DCM (1 mL) was stirred at room temperature for 16 h. Then to the mixture was added NaBH₄ (20.0 mg, 0.52 mmol) at room temperature. The resulting reaction was stirred for an additional 8 h, quenched with saturated aq. NH₄Cl (1 mL) and concentrated under reduced pressure. The residue was purified with Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 25% B to 50% B in 5.3 min; Detector: UV 254/210 nm; Retention time: 5.36 min. The fractions containing the desired product were collected and concentrated under reduced pressure to afford (2R,8aS)-2-(2,3-dichloro-6-hydroxyphenyl)-7-[(3R)-3-hydroxypyrrolidin-1-yl]-hexahydro-1H-indolizin-5-one as an off-white solid (12.9 mg, 28%): LCMS (ESI) calc'd for C₁₈H₂₂Cl₂N₂O₃ [M+H]⁺: 385, 387 (3:2) found 385, 387 (3:2); ¹H NMR (400 MHz, CD₃OD) δ 7.27 (d, J=8.8, 1H), 6.79 (d, J=8.8 Hz, 1H), 4.38-4.22 (m, 1H), 4.22-4.07 (m, 1H), 3.88-3.71 (m, 3H), 3.67-3.49 (m, 3H), 3.49-3.37 (m, 1H), 3.29-3.19 (m, 1H), 3.02-2.90 (m, 1H), 2.68 (dd, J=23.8, 11.8 Hz, 1H), 2.61-2.30 (m, 2H), 2.27-2.20 (m, 1H), 2.20-2.00 (m, 2H), 1.82-1.64 (m, 1H).

Step b:

(2R,8aS)-2-(2,3-dichloro-6-hydroxyphenyl)-7-[(3R)-3-hydroxypyrrolidin-1-yl]-hexahydro-1H-indolizin-5-one (12.9 mg, 0.03 mmol) was separated with Prep Chiral HPLC with the following conditions: Column: CHIRALPAK ID, 2×25 cm, 5 μm; Mobile Phase A: Hex (plus 0.2% FA)-HPLC, Mobile Phase B: EtOH-HPLC; Flow rate: 20 mL/min; Gradient: 20% B to 20% B in 12 min; Detector: UV 220/254 nm; Retention time 1: 6.66 min; Retention time 2: 8.97 min; Injection Volume: 1 mL; Number Of Runs: 2. The faster-eluting isomer at 6.66 min gave Compound 191 ((2R,8aS)-2-(2,3-dichloro-6-hydroxyphenyl)-7-[(3R)-3-hydroxypyrrolidin-1-yl]-hexahydro-1H-indolizin-5-one isomer 1) as an off-white solid (1.9 mg, 16.20%): LCMS (ESI) calc'd for C₁₈H₂₂Cl₂N₂O₃ [M+H]⁺: 385, 387 (3:2) found 385, 387 (3:2); ¹H NMR (400 MHz, CD₃OD) δ 8.50 (s, 1H), 7.26 (d, J=8.8 Hz, 1H), 6.76 (d, J=8.8 Hz, 1H), 4.52-4.36 (d, 1H), 4.36-4.19 (m, 1H), 4.12 (dd, J=11.6, 9.2 Hz, 1H), 3.88-3.73 (m, 1H), 3.58-3.42 (m, 1H), 3.29-3.09 (m, 3H), 3.09-2.91 (m, 2H), 2.83 (dd, J=17.3, 6.1 Hz, 1H), 2.57 (d, J=13.0 Hz, 1H), 2.50-2.30 (m, 2H), 2.28-2.15 (m, 2H), 1.97-1.88 (m, 1H), 1.57 (q, J=11.9 Hz, 1H). The slower-eluting isomer at 8.97 min gave Compound 192 ((2R, 8aS)-2-(2,3-dichloro-6-hydroxyphenyl)-7-[(3R)-3-hydroxypyrrolidin-1-yl]-hexahydro-1H-indolizin-5-one isomer 2) as a white solid (1.5 mg, 12.79% o): LCMS (ESI) calc'd for C₁₈H₂₂Cl₂N₂O₃ [M+H]⁺: 385, 387 (3:2) found 385, 387 (3:2); ¹H NMR (400 MHz, CD₃OD) δ 8.53 (s, 1H), 7.26 (d, J=8.8 Hz, 1H), 6.79 (d, J=8.8 Hz, 1H), 4.54-4.48 (m, 1H), 4.31-4.03 (m, 3H), 3.63-3.57 (m, 2H), 3.41-3.34 (m, 1H), 3.25-3.05 (m, 3H), 2.86 (dd, J=17.1, 5.9 Hz, 1H), 2.60-2.38 (m, 3H), 2.30-2.17 (m, 1H), 2.11 (q, J=10.6 Hz, 1H), 2.01-1.89 (m, 1H), 1.57 (q, J=11.9 Hz, 1H).

Example 67. Compounds 193-205 were prepared in an analogous fashion as that described for Compounds 191-192.

| Compound | | | |
|---|---|---|---|
| Number | Structure | Chemical Name | MS: (M + H)⁺ & ¹H MNR |
| 193 | 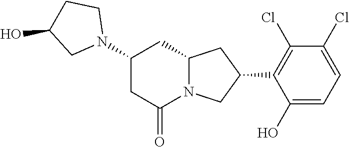 [SMILES: O=C1C[C@H](N2CC[C@H](O)C2)C[C@@H]2C[C@H](c3c(O)ccc(Cl)c3Cl)CN12] | (2R,8aS)-2-(2,3- dichloro-6- hydroxyphenyl)-7- [(3S)-3- hydroxypyrrolidin- 1-yl]-hexahydro- 1H-indolizin-5-one isomer 1 | [M + H]⁺: 385, 387 (3:2); ¹H NMR (400 MHz, CD₃OD) δ 8.52-8.46 (brs, 1H), 7.26 (d, J = 8.8 Hz, 1H), 6.76 (d, J = 8.8 Hz, 1H), 4.52-4.45 (m, 1H), 4.34-4.24 (m, 1H), 4.12 (dd, J = 11.6, 9.2 Hz, 1H), 3.83-3.73 (m, 1H), 3.62-3.48 (m, 1H), 3.30-3.13 (m, 3H), 3.08-3.00 (m, 2H), 2.84 (dd, J = 17.5, |
| | | | 6.1 Hz, 1H), 2.58 (d, J = 12.4 |
| | | | Hz, 1H), 2.50-2.37 (m, 2H), |
| | | | 2.27-2.17 (m, 2H), 1.97-1.88 |
| | | | (m, 1H), 1.60-1.50 (m, 1H). |
| 194 | 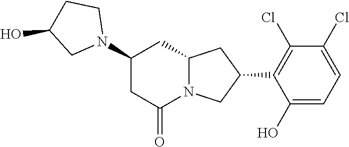 [SMILES: O=C1C[C@@H](N2CC[C@H](O)C2)C[C@@H]2C[C@H](c3c(O)ccc(Cl)c3Cl)CN12] | (2R,8aS)-2-(2,3- dichloro-6- hydroxyphenyl)-7- [(3S)-3- hydroxypyrrolidin- 1-yl]-hexahydro- 1H-indolizin-5-one isomer 2 | [M + H]⁺: 385, 387 (3:2); ¹H NMR (400 MHz, CD₃OD) δ 8.60-8.55 (brs, 1H), 7.25 (d, J = 8.7 Hz, 1H), 6.78 (d, J = 8.8 Hz, 1H), 4.50-4.43 (m, 1H), 4.27-4.06 (m, 3H), 3.58 (dd, J = 10.8, 7.0 Hz, 1H), 3.22-3.05 (m, 3H), 3.01-2.90 (m, 2H), 2.79 (dd, J = 17.2, 6.0 Hz, 1H), 2.54-2.43 (m, 2H), 2.41- |
| | | | 2.33 (m, 1H), 2.24-2.05 (m, |
| | | | 2H), 1.94-1.84 (m, 1H), 1.57- |
| | | | 1.46 (m, 1H). |
| 195 | 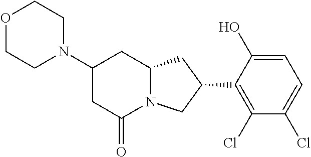 [SMILES: O=C1CC(N2CCOCC2)C[C@@H]2C[C@H](c3c(O)ccc(Cl)c3Cl)CN12] | (2R,8aS)-2-(2,3- dichloro-6- hydroxyphenyl)-7- (morpholin-4-yl)- hexahydro-1- indolizin-5-one | [M + H]⁺: 385, 387 (3:2); ¹H NMR (400 MHz, CD₃OD) δ 7.25 (d, J = 8.7 Hz, 1H), 6.77 (d, J = 8.7 Hz, 1H), 4.34-4.02 (m, 2H), 3.77-3.70 (m, 5H), 3.62-3.46 (m, 1H), 2.94-2.82 (m, 1H), 2.72-2.56 (m, 5H), 2.51-2.28 (m, 3H), 2.23-2.02 (m, 1H), 1.45-1.34 (m, 1H). |
| 196 | 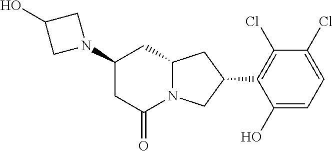 [SMILES: O=C1C[C@@H](N2CC(O)C2)C[C@@H]2C[C@H](c3c(O)ccc(Cl)c3Cl)CN12] | (2R,8aS)-2-(2,3- dichloro-6- hydroxyphenyl)-7- (3- hydroxyazetidin-1- yl)-hexahydro-1H- indolizin-5-one isomer 1 | [M + H]⁺: 371, 373 (3:2); ¹H NMR (400 MHz, CD₃OD) δ 8.37 (s, 1H), 7.25 (d, J = 8.8 Hz, 1H), 6.78 (d, J = 8.8 Hz, 1H), 4.52-4.45 (m, 1H), 4.27- 4.05 (m, 3H), 3.97-3.93 (m, 2H), 3.57 (dd, J = 11.0, 7.3 Hz, 1H), 3.39-3.35 (m, 1H), 3.18-3.05 (m, 1H), 2.69 (dd, J = 17.1, 6.0 Hz, 1H), 2.51-2.44 |
| | | | (m, 1H), 2.35 (d, J = 12.4 Hz, |
| | | | 1H), 2.16-2.01 (m, 2H), 1.39- |
| | | | 1.16 (m, 2H). |
| 197 | 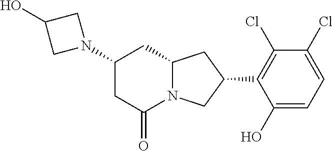 [SMILES: O=C1C[C@H](N2CC(O)C2)C[C@@H]2C[C@H](c3c(O)ccc(Cl)c3Cl)CN12] | (2R,8aS)-2-(2,3- dichloro-6- hydroxyphenyl)-7- (3- hydroxyazetidin-1- yl)-hexahydro-1H- indolizin-5-one isomer 2 | [M + H]⁺: 371, 373 (3:2); ¹H NMR (400 MHz, CD₃OD) δ 8.37 (s, 1H), 7.25 (d, J = 8.8 Hz, 1H), 6.75 (d, J = 8.8 Hz, 1H), 4.53-4.46 (m, 1H), 4.32- 4.23 (m, 1H), 4.10 (dd, J = 11.7, 9.1 Hz, 1H), 4.01-3.92 (m, 2H), 3.81-3.71 (m, 1H), 3.55-3.49 (m, 1H), 3.41-3.35 (m, 1H), 3.15-3.06 (m, 1H), |
| | | | 2.72 (dd, J = 17.3, 6.1 Hz, |
| | | | 1H), 2.47-2.34 (m, 2H), 2.33- |
| | | | 2.05 (m, 2H), 1.37-1.24 (m, |
| | | | 2H). |
| 198 | 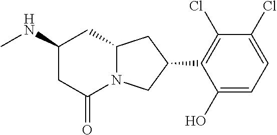 [SMILES: CN[C@@H]1CC(=O)N2C[C@@H](c3c(O)ccc(Cl)c3Cl)C[C@H]2C1] | (2R,8aS)-2-(2,3- dichloro-6- hydroxyphenyl)-7- (methylamino)- hexahydro-1H- indolizin-5-one isomer 1 | [M + H]⁺: 329, 331 (3:2); ¹H NMR (400 MHz, CD₃OD) δ 7.25 (d, J = 8.8 Hz, 1H), 6.75 (d, J = 8.8 Hz, 1H), 4.34-4.20 (m, 1H), 4.11 (dd, J = 11.7, 9.1 Hz, 1H), 3.82-3.72 (m, 1H), 3.54-3.48 (m, 1H), 3.05- 2.97 (m, 1H), 2.76 (dd, J = 17.8, 5.8 Hz, 1H), 2.45 (s, |
| | | | 3H), 2.49-2.33 (m, 2H), 2.33- |
| | | | 2.07 (m, 2H), 1.36-1.27 (m, |
| | | | 1H). |
| 199 | 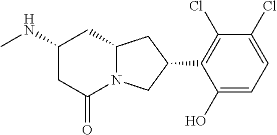 [SMILES: CN[C@H]1CC(=O)N2C[C@@H](c3c(O)ccc(Cl)c3Cl)C[C@H]2C1] | (2R,8aS)-2-(2,3- dichloro-6- hydroxyphenyl)-7- (methylamino)- hexahydro-1H- indolizin-5-one isomer 2 | [M + H]⁺: 329, 331 (3 : 2); ¹H NMR (400 MHz, CD₃OD) δ 7.27 (d, J = 8.8 Hz, 1H), 6.77 (d, J = 8.8 Hz, 1H), 4.35-4.23 (m, 1H), 4.18 (dd, J = 11.6, 9.0 Hz, 1H), 3.85-3.75 (m, 2H), 3.62-3.56 (m, 1H), 2.90 (dd, J = 18.4, 6.3 Hz, 1H), 2.82 (s, 3H), 2.66-2.61 (m, |
| | | | 1H), 2.57-2.39 (m, 2H), 2.31- |
| | | | 2.24 (m, 1H), 2.07-1.95 (m, |
| | | | 1H). |
| 200 | 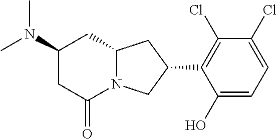 [SMILES: CN(C)[C@@H]1CC(=O)N2C[C@@H](c3c(O)ccc(Cl)c3Cl)C[C@H]2C1] | (2R,8aS)-2-(2,3- dichloro-6- hydroxyphenyl)-7- (dimethylamino)- hexahydro-1H- indolizin-5-one isomer 1 | [M + H]⁺: 343, 345 (3:2); ¹H NMR (400 MHz, CD₃OD) δ 7.25 (d, J = 8.8 Hz, 1H), 6.78 (d, J = 8.8 Hz, 1H), 4.32-4.22 (m, 1H), 4.11 (dd, J = 11.7, 9.2 Hz, 1H), 3.80-3.70 (m, 1H), 3.54-3.48 (m 1H), 2.92- 2.84 (m, 1H), 2.65 (dd, J = 17.5, 5.9 Hz, 1H), 2.46-2.28 |
| | | | (m, 9H), 2.23-2.15 (m, 1H), |
| | | | 1.45-1.36 (m, 1H). |
| 201 | 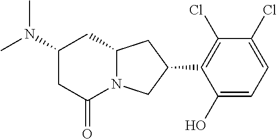 [SMILES: CN(C)[C@H]1CC(=O)N2C[C@@H](c3c(O)ccc(Cl)c3Cl)C[C@H]2C1] | (2R,8aS)-2-(2,3- dichloro-6- hydroxyphenyl)-7- (dimethylamino)- hexahydro-1H- indolizin-5-one isomer 2 | [M + H]⁺: 343, 345 (3:2); ¹H NMR (400 MHz, CD₃OD) δ 7.25 (d, J = 8.8 Hz, 1H), 6.78 (d, J = 8.8 Hz, 1H), 4.25-4.04 (m, 3H), 3.58 (dd, J = 10.9, 7.1Hz, 1H), 3.01-2.92 (m, 1H), 2.65 (dd, J = 17.1, 6.1 Hz, 1H), 2.50-2.31 (m, 9H), 2.15-2.02 (m, 1H), 1.48-1.38 |
| | | | (m, 1H). |
| 202 | 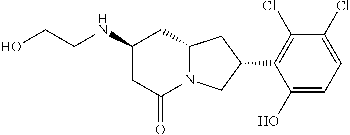 [SMILES: O=C1C[C@@H](NCCO)C[C@@H]2C[C@H](c3c(O)ccc(Cl)c3Cl)CN12] | (2R,8aS)-2-(2,3- dichloro-6- hydroxyphenyl)-7- [(2- hydroxyethyl)amino]- hexahydro-1H- indolizin-5-one isomer 1 | [M + H]⁺: 359, 361 (3:2); ¹H NMR (400 MHz, CD₃OD) δ 7.24 (d, J = 8.8 Hz, 1H), 6.75 (d, J = 8.8 Hz, 1H), 4.34-4.22 (m, 1H), 4.11 (d, J = 11.6, 9.1 Hz, 1H), 3.85-3.65 (m, 3H), 3.55-3.48 (m, 1H), 3.19-3.07 (m, 1H), 2.84-2.72 (m, 3H), 2.50-2.34 (m, 2H), 2.22-2.11 |
| | | | (m, 2H), 1.41-1.28 (m, 1H). |
| 203 | 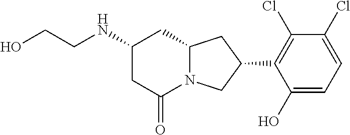 [SMILES: O=C1C[C@H](NCCO)C[C@@H]2C[C@H](c3c(O)ccc(Cl)c3Cl)CN12] | (2R,8aS)-2-(2,3- dichloro-6- hydroxyphenyl)-7- [(2- hydroxyethyl)amino]- hexahydro-1H- indolizin-5-one isomer 2 | [M+H] 359, 361 (3:2); ¹H NMR (400 MHz, CD₃OD) δ 7.25 (d, J = 8.9 Hz, 1H), 6.78 (d, J = 8.7 Hz, 1H), 4.27-4.05 (m, 3H), 3.72-3.65 (m, 2H), 3.62-3.52 (m, 1H), 3.20-3.07 (m, 1H), 2.90-2.66 (m, 3H), 2.54-2.33 (m, 2H), 2.20-2.00 (m, 2H), 1.37-1.25 (m, 1H). |
| 204 | 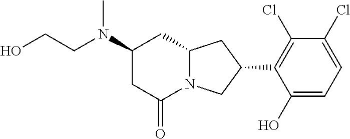 [SMILES: CN(CCO)[C@@H]1CC(=O)N2C[C@@H](c3c(O)ccc(Cl)c3Cl)C[C@H]2C1] | (2R,8aS)-2-(2,3- dichloro-6- hydroxyphenyl)-7- [(2- hydroxyethyl) (methyl)amino]- hexahydro-1H- indolizin-5-one isomer 1 | [M + H]⁺: 373, 375 (3:2); ¹H NMR (400 MHz, CD₃OD) δ 7.25 (d, J = 8.8 Hz, 1H), 6.77 (d, J = 8.8 Hz, 1H), 4.28-4.03 (m, 3H), 3.69-3.64 (m, 2H), 3.61-3.54 (m, 1H), 3.21-3.09 (m, 1H), 2.72-2.55 (m, 3H), 2.51-2.27 (m, 6H), 2.13-2.01 (m, 1H), 1.54-1.40 (m, 1H). |
| 205 | 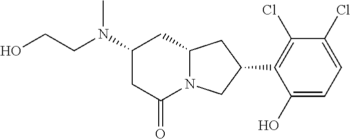 [SMILES: CN(CCO)[C@H]1CC(=O)N2C[C@@H](c3c(O)ccc(Cl)c3Cl)C[C@H]2C1] | (2R,8aS)-2-(2,3- dichloro-6- hydroxyphenyl)-7- [(2- hydroxyethyl) (methyl)amino]- hexahydro-1H- indolizin-5-one isomer 2 | [M + H]⁺: 373, 375 (3:2); ¹H NMR (400 MHz, CD₃OD) δ 7.25 (d, J = 9.0 Hz, 1H), 6.76 (d, J = 9.0 Hz, 1H), 4.36-4.17 (m, 1H), 4.17-4.04 (m, 1H), 3.82-3.62 (m, 3H), 3.54-3.47 (m, 1H), 3.23-3.06 (m, 1H), 2.76-2.54 (m, 3H), 2.50-2.29 (m, 6H), 2.23-2.11 (m, 1H), |
| | | | 1.54-1.39 (m, 1H). |

### Example 68. Compound 206 (N-[(2R,8aS)-2-(2,3-dichloro-6-hydroxyphenyl)-5-oxo-hexahydro-1H-indolizin-7-yl]acetamide))

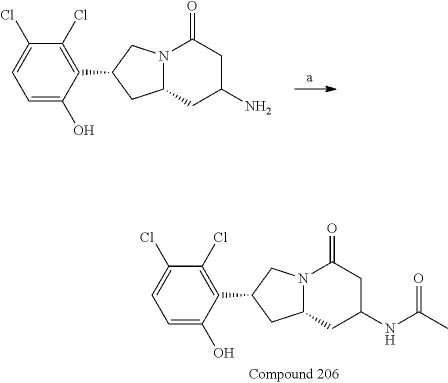

Step a:

To a stirred solution of (2R,8aS)-7-amino-2-(2,3-dichloro-6-hydroxyphenyl)-hexahydro-1H-indolizin-5-one (Example 65, step c) (50.0 mg, 0.16 mmol) and TEA (48.0 mg, 0.47 mmol) in DCM (1 mL) was added acetyl chloride (12 mg, 0.16 mmol) at room temperature. The resulting mixture was stirred for 2 hand then concentrated under reduced pressure. The residue was purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 30% B to 50% B in 5.5 min; Detector: UV 210 nm; Retention time: 5.5 min. The fractions containing the desired product were collected and concentrated under reduced pressure to afford Compound 206 (N-[(2R,8aS)-2-(2,3-dichloro-6-hydroxyphenyl)-5-oxo-hexahydro-1H-indolizin-7-yl]acetamide) as an off-white solid (23.0 mg, 39%): LCMS (ESI) calc'd for C₁₆H₁₈Cl₂N₂O₃ [M+H]⁺: 357, 359 (3:2) found 357, 359 (3:2); ¹H NMR (400 MHz, CD₃OD) δ 7.25 (d, J=8.8 Hz, 1H), 6.76 (d, J=8.8 Hz, 1H), 4.39-4.32 (m, 1H), 4.32-4.21 (m, 1H), 4.16 (dd, J=11.5, 9.0 Hz, 1H), 3.91-3.80 (m, 1H), 3.55 (dd, J=11.5, 9.8 Hz, 1H), 2.73 (dd, J=18.3, 6.3 Hz, 1H), 2.46-2.29 (m, 3H), 2.20-2.12 (m, 1H), 1.99 (s, 3H), 1.71-1.61 (m, 1H).

### Example 69. Compound 207) ((2R,7S,8aS)-2-(2,3-dichloro-6-hydroxyphenyl)-7-(piperazin-1-yl)hexahydroindolizin-5(1H)-one) and Compound 208 ((2R,7R,8aS)-2-(2,3-dichloro-6-hydroxyphenyl)-7-(piperazin-1-yl)hexahydroindolizin-5(1H)-one)

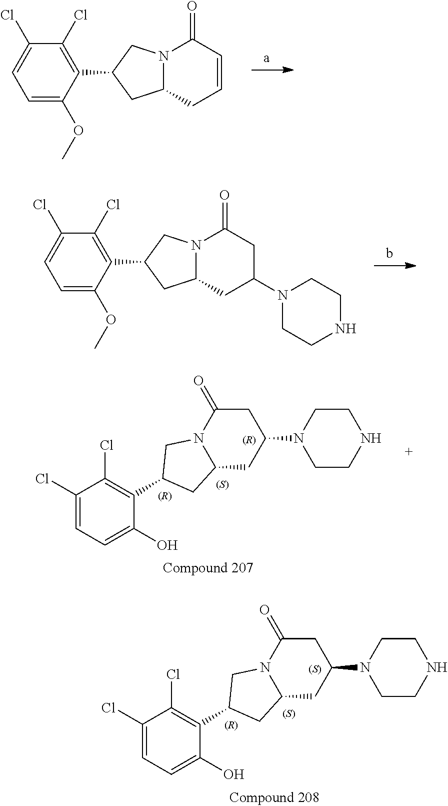

Step a:

To a stirred mixture of (2R,8aR)-2-(2,3-dichloro-6-methoxyphenyl)-2,3,8,8a-tetrahydro-1H-indolizin-5-one (Intermediate 17, Example 15) (0.300 g, 0.96 mmol) in H₂O (0.50 mL) was added piperazine (0.830 g, 9.61 mmol) at room temperature. The resulting mixture was stirred at 90° C. for 16 h, cooled down to room temperature and concentrated under reduced pressure. The residue was purified by reverse phase chromatography, eluting with 30% ACN in water (plus 0.05% TFA) to afford (2R,8aS)-2-(2,3-dichloro-6-methoxyphenyl)-7-(piperazin-1-yl)-hexahydro-1H-indolizin-5-one as a light yellow oil (0.300 g, 60%): LCMS (ESI) calc'd for C₁₉H₂₅Cl₂N₃O₂ [M+H]⁺: 398, 400 (3:2) found 398, 400 (3:2); ¹H NMR (300 MHz, CDCl₃) δ 7.33 (d, J=8.6 Hz, 1H), 6.76 (d, J=8.9 Hz, 1H), 4.36-4.14 (m, 1H), 4.08-3.85 (m, 1H), 3.78 (s, 3H), 3.76-3.61 (m, 8H), 3.61-3.46 (m, 3H), 3.12-2.53 (m, 3H), 2.42-2.12 (m, 3H).

Step b:

To a stirred mixture of (2R,8aS)-2-(2,3-dichloro-6-methoxyphenyl)-7-(piperazin-1-yl)-hexahydro-1H-indolizin-5-one (0.300 g, 1.17 mmol) in DCM (5 mL) was added BBr₃ (1.00 mL) dropwise at room temperature. The resulting reaction was stirred at room temperature for 1 h, quenched with MeOH (5 mL) at 0° C. and concentrated under reduced pressure. The residue was purified by reverse phase chromatography, eluting with 30% ACN in water (plus 0.05% TFA) to afford the desired product. The products were separated by Prep Chiral HPLC with the following conditions: Column: CHIRALPAK IG, 2×25 cm, 5 m; Mobile Phase A: Hex (plus 0.2% IPA)-HPLC, Mobile Phase B: EtOH-HPLC; Flow rate: 20 mL/min; Gradient: 20% B to 20% B in 14 min; Detector UV 220/254 nm; Retention Time 1: 7.51 min; Retention Time 2: 11.52 min; Injection Volume: 1.2 mL; Number Of Runs: 1. The faster-eluting isomer at 7.51 min was Compound 207 ((2R,7S,8aS)-2-(2,3-dichloro-6-hydroxyphenyl)-7-(piperazin-1-yl)-hexahydro-1H-indolizin-5-one) as an off-white solid (76.5 mg, 16%): LCMS (ESI) calc'd C₁₈H₂₃Cl₂N₃O₂ for [M+H]⁺: 384, 386 (3:2) found 384, 386 (3:2): ¹H NMR (400 MHz, CD₃OD) δ 7.24 (d, J=8.8 Hz, 1H), 6.75 (d, J=8.8 Hz, 1H), 4.31-4.19 (m, 1H), 4.12 (dd, J=11.5, 8.8 Hz, 1H), 3.96-3.83 (m, 1H), 3.55-3.46 (m, 1H), 2.91 (t, J=5.0 Hz, 4H), 2.79-2.71 (m, 1H), 2.68-2.54 (m, 5H), 2.53-2.41 (m, 2H), 2.37-2.30 (m, 1H), 2.21-2.10 (m, 1H), 1.73-1.62 (m, 1H). The slower-eluting isomer at 11.52 min was Compound 208 ((2R,7R,8aS)-2-(2,3-dichloro-6-hydroxyphenyl)-7-(piperazin-1-yl)-hexahydro-1H-indolizin-5-one) as an off-white solid (72.3 mg, 15%). LCMS (ESI) calc'd C₁₈H₂₃Cl₂N₃O₂ for [M+H]⁺: 384, 386 (3:2) found 384, 386 (3:2); ¹H NMR (400 MHz, CD₃OD) δ 7.25 (d, J=8.8 Hz, 1H), 6.75 (d, J=8.8 Hz, 1H), 4.34-4.21 (m, 1H), 4.16-4.05 (m, 1H), 3.80-3.68 (m, 1H), 3.51 (dd, J=11.6, 9.8 Hz, 1H), 2.97-2.87 (m, 5H), 2.73-2.56 (m, 5H), 2.49-2.29 (m, 3H), 2.21-2.13 (m, 1H), 1.46-1.39 (m, 1H).

### Example 70. Compound 213 ((2R, 8aS)-2-(2,3-dichloro-6-hydroxyphenyl)-7-(1H-pyrazol-3-yl)-hexahydro-1H-indolizin-5-one isomer 1) and Compound 214 ((2R,8aS)-2-(2,3-dichloro-6-hydroxyphenyl)-7-(1H-pyrazol-3-yl)-hexahydro-1H-indolizin-5-one isomer 2)

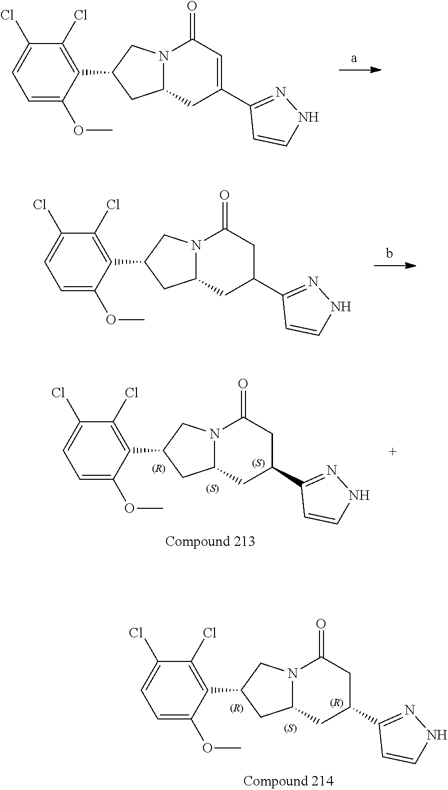

Step a:

To a stirred solution of (2R,8aS)-2-(2,3-dichloro-6-methoxyphenyl)-7-(2H-pyrazol-3-yl)-2,3,8,8a-tetrahydro-1H-indolizin-5-one (70.0 mg, 0.19 mmol) in MeOH (2 mL), EA (2 mL) and AcOH (0.50 mL) was added PtO₂ (42.0 mg, 0.19 mmol) at room temperature. The reaction was stirred at room temperature for 16 h under hydrogen atmosphere (1.5 atm). The resulting mixture was filtered and the filtrate was concentrated under reduced pressure. The residue was purified by reverse phase chromatography, eluting with 40% ACN in water (plus 0.1% FA) to afford (2R,8aS)-2-(2,3-dichloro-6-methoxyphenyl)-7-(2H-pyrazol-3-yl)-hexahydro-1H-indolizin-5-one as a light yellow solid (40 mg, 57%): LCMS (ESI) calc'd for C₁₈H₁₉Cl₂N₃O₂ [M+H]⁺380, 382 (3:2) found 380, 382 (3:2).

Step b:

To a stirred solution of (2R,8aS)-2-(2,3-dichloro-6-methoxyphenyl)-7-(2H-pyrazol-3-yl)-2,3,8,8a-tetrahydro-1H-indolizin-5-one (35.0 mg, 0.09 mmol) in DCM (2 mL) was added BBr₃ (0.140 g, 0.55 mmol) at room temperature. The reaction was stirred for 1 h, quenched with MeOH (2 mL) and concentrated under reduced pressure. The residue was purified by Prep-HPLC with the following conditions: X Bridge Shield RP18 OBD Column, 19×250 mm, 10 μm; Mobile Phase A: Water (plus 10 mM NH₄HCO₃), Mobile Phase B: ACN; Flow rate: 20 mL/min; Gradient: 35% B to 60% B in 5.5 min; Detector: UV 224 nm; Retention Time: 5.56 min. The fractions containing the desired product were collected and concentrated under reduced pressure to afford the mixture product. The product was then separated with Prep Chiral HPLC with the following conditions: Column: CHIRALPAK ID-2, 2×25 cm, 5 μm; Mobile Phase A: Hex (plus 0.1% FA)-HPLC, Mobile Phase B: IPA-HPLC; Flow rate: 15 mL/min; Gradient: 50% B to 50% B in 25 min; Detector: UV 224 nm; Retention Time 1: 9.91 min; Retention Time 2: 18.38 min; Injection Volume: 2 mL; Number of Runs: 2. The faster-eluting isomer at 9.91 min was obtained Compound 213 ((2R,8aS)-2-(2,3-dichloro-6-hydroxyphenyl)-7-(1H-pyrazol-3-yl)-hexahydro-1H-indolizin-5-one isomer 1) as an off-white solid (3.8 mg, 11%): LCMS (ESI) calc'd for C₁₇H₁₇Cl₂N₃O₂ [M+H]⁺ 366, 368 (3:2) found 366, 368 (3:2); ¹H NMR (400 MHz, DMSO-d₆) δ 7.52 (d, J=2.2 Hz, 1H), 7.32 (d, J=8.8 Hz, 1H), 6.84 (d, J=8.8 Hz, 1H), 6.17 (d, J=2.2 Hz, 1H), 4.14-3.91 (m, 3H), 3.37 (dd, J=10.9, 7.7 Hz, 1H), 3.27-3.15 (m, 1H), 2.64-2.54 (m, 1H), 2.37-2.22 (m, 3H), 2.02-1.88 (m, 1H), 1.56-1.50 (m, 1H). The slower-eluting isomer at 18.38 min was obtained Compound 214 ((2R,8aS)-2-(2,3-dichloro-6-hydroxyphenyl)-7-(1H-pyrazol-3-yl)-hexahydro-1H-indolizin-5-one isomer 2) as an off-white solid (9.3 mg, 28%): LCMS (ESI) calc'd for C₁₇H₁₇Cl₂N₃O₂ [M+H]⁺ 366, 368 (3:2) found 366, 368 (3:2); ¹H NMR (400 MHz, DMSO-d₆) δ 7.54 (d, J=2.2 Hz, 1H), 7.33 (d, J=8.8 Hz, 1H), 6.82 (d, J=8.8 Hz, 1H), 6.17 (d, J=2.2 Hz, 1H), 4.13-3.98 (m, 1H), 3.98-3.88 (m, 1H), 3.88-3.74 (m, 1H), 3.40 (dd, J=10.6, 7.7 Hz, 1H), 3.32-3.16 (m, 1H), 2.60 (dd, J=17.8, 5.8 Hz, 1H), 2.36-2.25 (m, 2H), 2.23-2.15 (m, 1H), 2.13-2.03 (m, 1H), 1.55-1.46 (m, 1H).

Example 71. Compounds 215-216 were prepared in an analogous fashion as that described for Compounds 213-214.

| Compound | | | |
|---|---|---|---|
| Number | Structure | Chemical Name | MS: (M + H)⁺ & ¹H MNR |
| 215 | 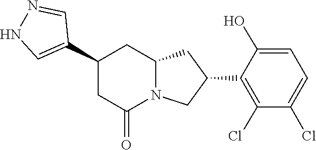 [SMILES: O=C1C[C@@H](c2cn[nH]c2)C[C@@H]2C[C@H](c3c(O)ccc(Cl)c3Cl)CN12] | (2R,8aR)-2-(2,3- dichloro-6- hydroxyphenyl)-7- (1H-pyrazol-4-yl)- hexahydro-1H- indolizin-5-one isomer 1 | [M + H]⁺: 366, 368 (3:2); ¹H NMR (400 MHz, DMSO- d₆) δ 7.53 (s, 2H), 7.33 (d, J = 8.8 Hz, 1H), 6.82 (d, J = 8.9 Hz, 1H), 4.12-4.01 (m, 1H), 3.97-3.88 (m, 1H), 3.85-3.72 (m, 1H), 3.43-3.37 (m, 1H), 3.14-3.03 (m, 1H), 2.67-2.53 (m, 1H), 2.34-2.07 |
| | | | (m, 4H), 1.48-1.38 (m, 1H). |
| 216 | 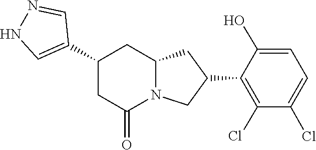 | (2R,8aR)-2-(2,3- dichloro-6- hydroxyphenyl)-7- (1H-pyrazol-4-yl)- hexahydro-1H- indolizin-5-one isomer 2 | [M + H]⁺: 366, 368 (3:2); ¹H NMR (400 MHz, DMSO- d₆) δ 7.51 (s, 2H), 7.33 (d, J = 8.8 Hz, 1H), 6.85 (d, J = 8.8 Hz, 1H), 4.13-3.93 (m, 3H), 3.39-3.30 (m, 1H), 3.13-3.05 (m, 1H), 2.63-2.52 (m, 1H), 2.35-2.17 (m, 3H), 2.00-1.91 (m, 1H), 1.52-1.42 (m, 1H). |

### Example 72. Compound 217 ((2R,8aR)-7-(aminomethyl)-2-(2,3-dichloro-6-hydroxyphenyl)-hexahydro-1H-indolizin-5-one)

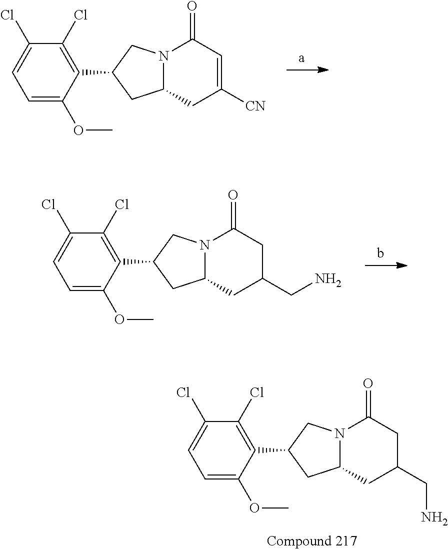

Step a:

To a stirred solution of (2R,8aS)-2-(2,3-dichloro-6-methoxyphenyl)-5-oxo-2,3,8,8a-tetrahydro-1H-indolizine-7-carbonitrile (70.0 mg, 0.21 mmol) in MeOH (3 mL) and AcOH (3 mL) was added PtO₂ (47.0 mg, 0.21 mmol) at room temperature. The reaction was stirred for 1 h under hydrogen atmosphere (1.5 atm). The resulting mixture was filtered and the filtrate concentrated under reduced pressure. The residue was purified by reverse phase chromatography, eluting with 35% ACN in water (plus 0.05% TFA) to afford (2R,8aR)-7-(aminomethyl)-2-(2,3-dichloro-6-methoxyphenyl)-hexahydro-1H-indolizin-5-one as a colorless oil (50.0 mg, 53%): LCMS (ESI) calc'd for C₁₆H₂OCl₂N₂O₂ [M+H]⁺ 343, 345 (3:2) found 343, 345 (3:2); ¹H NMR (300 MHz, CD₃OD) δ 7.43 (dd, J=9.0, 1.5 Hz, 1H), 7.06-6.95 (m, 1H), 4.42-4.13 (m, 1H), 4.12-3.96 (m, 1H), 3.92-3.76 (m, 4H), 3.57-3.45 (m, 1H), 3.12-2.92 (m, 2H), 2.68-2.49 (m, 1H), 2.36-2.04 (m, 5H), 1.37-1.26 (m, 1H).

Step b:

To a stirred solution of (2R,8aR)-7-(aminomethyl)-2-(2,3-dichloro-6-methoxyphenyl)-hexahydro-1H-indolizin-5-one (50.0 mg, 0.11 mmol) in DCM (2 mL) was added BBr₃ (0.270 g, 1.09 mmol) at room temperature. The reaction was stirred at room temperature for 3 h, quenched with MeOH (1 mL) and concentrated under reduced pressure. The residue was purified by reverse phase chromatography, eluting with 45% ACN in water (plus 10 mM NH₄HCO₃) to afford the crude product. The crude was purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 20% B to 21% B in 5.5 min; Detector: UV 254/210 nm; Retention Time: 5.58 min. The fractions containing the desired product were collected and concentrated under reduced pressure to afford Compound 217 ((2R,8aR)-7-(aminomethyl)-2-(2,3-dichloro-6-hydroxyphenyl)-hexahydro-1H-indolizin-5-one formic acid) as an off-white solid (10.0 mg, 24.37%): LCMS (ESI) calc'd for C₁₅H₁₈Cl₂N₂O₂ [M+H]⁺ 329, 331 (3:2) found 329, 331 (3:2); ¹H NMR (400 MHz, CD₃OD) δ 8.54 (s, 1H), 7.25 (dd, J=8.8, 5.7 Hz, 1H), 6.76 (dd, J=8.8, 4.9 Hz, 1H), 4.43-4.23 (m, 1H), 4.23-4.03 (m, 1H), 3.91-3.77 (m, 1H), 3.63-3.45 (m, 1H), 3.43-3.36 (m, 1H), 2.98 (d, J=6.7 Hz, 1H), 2.59 (dd, J=17.3, 5.6 Hz, 1H), 2.50-2.37 (m, 1H), 2.32-2.01 (m, 4H), 1.38-1.30 (m, 1H).

### Example 73. Compound 218 ((2R,8aS)-7-(aminomethyl)-2-(2,3-dichloro-6-hydroxyphenyl)-7-hydroxy-hexahydroindolizin-5-one isomer 1) and Compound 219 ((2R,8aS)-7-(aminomethyl)-2-(2,3-dichloro-6-hydroxyphenyl)-7-hydroxy-hexahydroindolizin-5-one isomer 2)

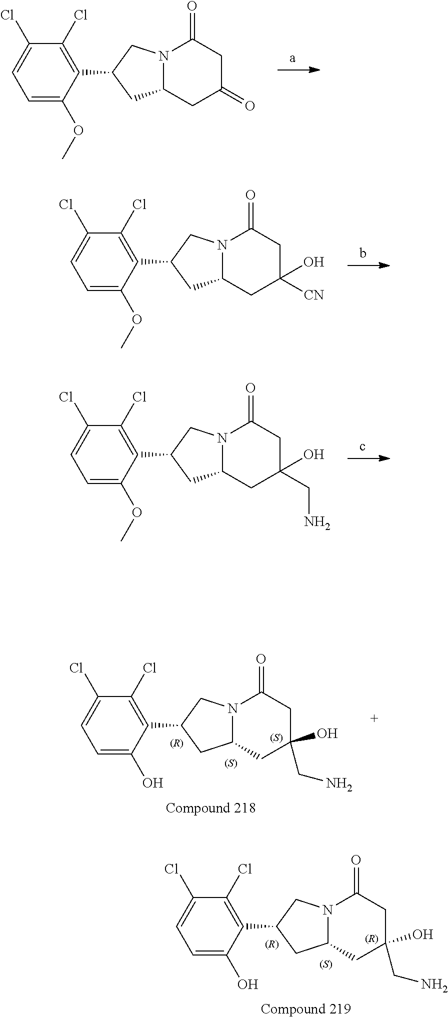

Step a:

To a stirred solution of (2R,8aS)-2-(2,3-dichloro-6-methoxyphenyl)-hexahydroindolizine-5,7-dione (Intermediate 15, Example 13) (0.500 g, 1.52 mmol) and ZnI₂ (0.150 g, 0.46 mmol) in DCE (6 mL) was added TMSCN (0.450 g, 4.57 mmol) at room temperature. The resulting reaction was stirred for 2 days at 80° C. and quenched with saturated aq. NaHCO₃ (20 mL) at room temperature followed by extraction with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 70% ACN in water (plus 0.05% TFA) to afford (2R,8aS)-2-(2,3-dichloro-6-methoxyphenyl)-7-hydroxy-5-oxooctahydroindolizine-7-carbonitrile (0.120 g, 22%) as a light yellow solid: LCMS (ESI) calc'd for C₁₆H₁₆Cl₂N₂O₃ [M+H]⁺: 355, 357 (3:2) found 355, 357 (3:2); ¹H NMR (400 MHz, CDCl₃) δ 7.37 (dd, J=8.9, 2.6 Hz, 1H), 6.84-6.67 (m, 1H), 4.29-4.08 (m, 2H), 3.85 (d, J=15.3 Hz, 3H), 3.66-3.55 (m, 1H), 3.31-3.16 (m, 1H), 2.72 (dt, J=50.1, 17.1 Hz, 2H), 2.46-2.06 (m, 2H), 1.95-1.80 (m, 1H), 1.78-1.42 (m, 1H).

Step b:

To a stirred solution of (2R,8aS)-2-(2,3-dichloro-6-methoxyphenyl)-7-hydroxy-5-oxooctahydroindolizine-7-carbonitrile (50.0 mg, 0.14 mmol) in MeOH (0.5 mL) were added AcOH (0.5 mL) and PtO₂ (6 mg) at room temperature. The resulting mixture was stirred for 2 h under hydrogen atmosphere. The mixture was filtered, the filter cake was washed with MeOH (3×5 mL) and the filtrate was concentrated under reduced pressure. The residue was purified by reverse phase chromatography, eluting with 35% ACN in water (plus 0.05% TFA) to afford (2R,8aS)-7-(aminomethyl)-2-(2,3-dichloro-6-methoxyphenyl)-7-hydroxy-hexahydroindolizin-5-one as an off-white solid (20.0 mg, 35%): LCMS (ESI) calc'd for C₁₆H₂OCl₂N₂O₃ [M+H]⁺: 359, 361 (3:2) found 359, 361 (3:2).

Step c:

To a stirred solution of (2R,8aS)-7-(aminomethyl)-2-(2,3-dichloro-6-methoxyphenyl)-7-hydroxy-hexahydroindolizin-5-one (20.0 mg, 0.06 mmol) in DCM (0.5 mL) was added BBr₃ (0.2 mL, 2.12 mmol) at room temperature. The reaction was stirred at room temperature for 1 h, quenched with MeOH (1 mL) at 0° C. and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 22% B to 27% B in 6.5 min; Detector: UV 210 nm; Retention time 1: 6.54 min; Retention time 2: 6.92 min. The faster-eluting isomer at 6.54 min was obtained Compound 218 ((2R,8aS)-7-(aminomethyl)-2-(2,3-dichloro-6-hydroxyphenyl)-7-hydroxy-hexahydroindolizin-5-one isomer 1) at 6.54 min as an off-white solid (5.7 mg, 21%): LCMS (ESI) calc'd for C₁₅H₁₈Cl₂N₂O₃ [M+H]⁺: 345, 347 (3:2), found 345, 347 (3:2); ¹H NMR (400 MHz, CD₃OD) δ 7.26 (d, J=8.8 Hz, 1H), 6.77 (d, J=8.8 Hz, 1H), 4.36-4.21 (m, 1H), 4.14 (dd, J=11.6, 9.0 Hz, 1H), 3.81-3.67 (m, 1H), 3.55-3.47 (m, 1H), 3.20-3.02 (m, 2H), 2.71-2.48 (m, 2H), 2.44-2.30 (m, 2H), 2.25-2.17 (m, 1H), 1.80 (dd, J=13.5, 11.5 Hz, 1H). The slower-eluting isomer at 6.92 min was obtained Compound 219 ((2R,8aS)-7-(aminomethyl)-2-(2,3-dichloro-6-hydroxyphenyl)-7-hydroxy-hexahydroindolizin-5-one isomer 1) as an off-white solid (2 mg, 7%): LCMS (ESI) calc'd for C₁₅H₁₈Cl₂N₂O₃ [M+H]⁺: 345, 347 (3:2) found 345, 347 (3:2); ¹H NMR (400 MHz, CD₃OD) δ 7.26 (d, J=8.8 Hz, 1H), 6.78 (d, J=8.8 Hz, 1H), 4.27-4.17 (m, 1H), 4.17-4.09 (m, 1H), 4.09-3.97 (m, 1H), 3.71-3.59 (m, 1H), 3.16-3.01 (m, 2H), 2.65-2.45 (m, 3H), 2.33-2.24 (m, 1H), 2.11-2.01 (m, 1H), 1.80 (dd, J=13.6, 11.4 Hz, 1H).

### Example 74. Compound 220 ((2R,8aS)-2-(2,3-dichloro-6-hydroxyphenyl)-7-hydroxy-7-(hydroxymethyl)-hexahydroindolizin-5-one)

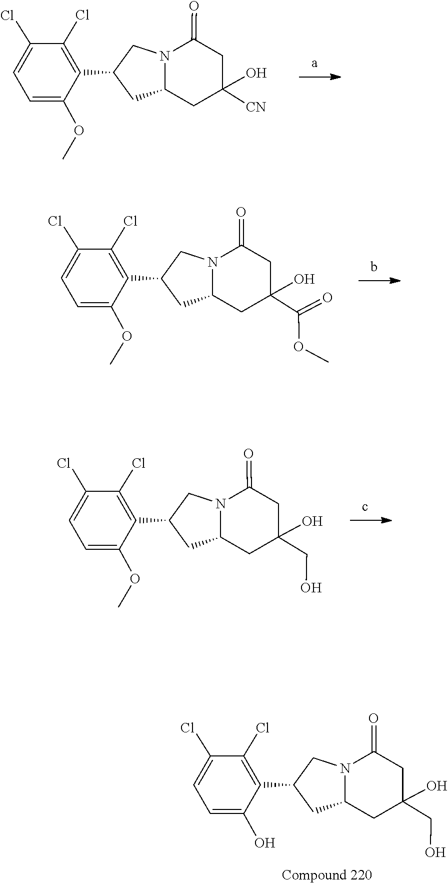 [SMILES: COc1ccc(Cl)c(Cl)c1[C@H]1C[C@H]2CC(O)(C#N)CC(=O)N2C1 ; COC(=O)C1(O)CC(=O)N2C[C@@H](c3c(OC)ccc(Cl)c3Cl)C[C@H]2C1 ; COc1ccc(Cl)c(Cl)c1[C@H]1C[C@H]2CC(O)(CO)CC(=O)N2C1 ; O=C1CC(O)(CO)C[C@@H]2C[C@H](c3c(O)ccc(Cl)c3Cl)CN12]

Step a:

To a stirred solution of (2R,8aS)-2-(2,3-dichloro-6-methoxyphenyl)-7-hydroxy-5-oxooctahydroindolizine-7-carbonitrile (Example 73, step a) (50.0 mg, 0.14 mmol) in MeOH (0.5 mL) was added SOCl₂ (0.25 mL, 4.02 mmol) at room temperature. The resulting mixture was stirred at room temperature for 2 h and concentrated under reduced pressure. The residue was purified by reverse phase chromatography, eluting with 50% ACN in water (plus 0.05% TFA) to afford methyl (2R,8aS)-2-(2,3-dichloro-6-methoxyphenyl)-7-hydroxy-5-oxo-hexahydroindolizine-7-carboxylate as an off-white solid (50 mg, 90%): LCMS (ESI) calc'd for C₁₇H₁₉Cl₂NO₅ [M+H]⁺: 388, 390 (3:2) found 388, 390 (3:2).

Step b:

To a stirred solution of methyl (2R,8aS)-2-(2,3-dichloro-6-methoxyphenyl)-7-hydroxy-5-oxo-hexahydroindolizine-7-carboxylate (50.0 mg, 0.13 mmol) in MeOH (1 mL) was added NaBH₄ (15.0 mg, 0.39 mmol) at room temperature. The resulting mixture was stirred at room temperature for 2 h, quenched with saturated aq. NH₄Cl (1 mL) and concentrated under reduced pressure. The residue was purified by reverse phase chromatography, eluting with 50% ACN in water (plus 0.05% TFA) to afford (2R,8aS)-2-(2,3-dichloro-6-methoxyphenyl)-7-hydroxy-7-(hydroxymethyl)-hexahydroindolizin-5-one as an off-white solid (40.0 mg, 73%): LCMS (ESI) calc'd for C₁₆H₁₉Cl₂NO₄ [M+H]⁺: 360, 362 (3:2) found 360, 362 (3:2); ¹H NMR (400 MHz, CDCl₃) δ 7.36 (dd, J=8.9, 3.9 Hz, 1H), 6.87-6.63 (m, 1H), 4.49-4.03 (m, 4H), 3.84 (s, 3H), 3.73-3.43 (m, 3H), 2.86-2.59 (m, 2H), 2.50-2.16 (m, 2H), 1.88-1.63 (m, 1H).

Step c:

To a stirred solution of (2R,8aS)-2-(2,3-dichloro-6-methoxyphenyl)-7-hydroxy-7-(hydroxymethyl)-hexahydroindolizin-5-one (40.0 mg, 0.11 mmol) in DCM (1 mL) was added BBr₃ (0.30 mL, 3.17 mmol) at room temperature. The resulting mixture was stirred at room temperature for 2 h, quenched with MeOH (5 mL) at 0° C. and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 30% B to 45% B in 6.5 min; Detector: UV 254/210 nm; Retention time: 6.54 min. The fractions containing the desired product were collected and concentrated under reduced pressure to afford Compound 220 ((2R,8aS)-2-(2,3-dichloro-6-hydroxyphenyl)-7-hydroxy-7-(hydroxymethyl)-hexahydroindolizin-5-one) as an off-white solid (13.0 mg, 33%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.25 (d, J=8.8 Hz, 1H), 6.76 (d, J=8.8 Hz, 1H), 4.32-4.15 (m, 1H), 4.15-4.01 (m, 1H), 3.81-3.61 (m, 1H), 3.61-3.42 (m, 3H), 2.63-2.48 (m, 1H), 2.48-2.29 (m, 3H), 2.22-1.98 (m, 1H), 1.68-1.53 (m, 1H).

### Example 75. Compound 223 ((2R,8aR)-2-(2,3-dichloro-6-hydroxyphenyl)-hexahydro-1H-indolizin-5-one)

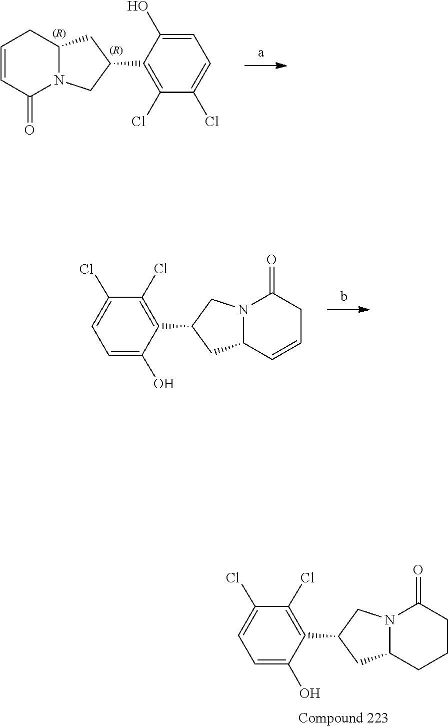

Step a:

Compound (2R,8aR)-2-(2,3-dichloro-6-hydroxyphenyl)-2,3,8,8a-tetrahydro-1H-indolizin-5-one was prepared in an analogous fashion to an example disclosed herein and/or analogous to known methods in the art. [M+H]⁺: 298, 300 (3:2); ¹H NMR (400 MHz, DMSO-d₆) δ 10.34 (s, 1H), 7.35 (d, J=8.8 Hz, 1H), 6.84 (d, J=8.9 Hz, 1H), 6.68-6.60 (m, 1H), 5.81 (d, J=9.8 Hz, 1H), 4.10-4.01 (m, 1H), 3.91-3.78 (m, 2H), 3.53 (dd, J=11.1, 9.7 Hz, 1H), 2.57 (dd, J=11.6, 5.8 Hz, 1H), 2.44-2.33 (m, 1H), 2.21-2.09 (m, 2H).

Step b:

To a stirred mixture of (2R,8aS)-2-(2,3-dichloro-6-hydroxyphenyl)-2,3,6,8a-tetrahydro-1H-indolizin-5-one (50.0 mg, 0.17 mmol) in MeOH (2 mL) was added PtO₂ (10.0 mg, 0.04 mmol) at room temperature. The reaction was stirred for 2 h under hydrogen atmosphere (1.5 atm). The resulting mixture was filtered and the filter cake was washed with MeOH (3×5 mL). The filtrate was concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 35% B to 65% B in 5.5 min; Detector: UV 210 nm; Retention time: 5.56 min. The fractions containing the desired product were collected and concentrated under reduced pressure to afford Compound 223 ((2R,8aR)-2-(2,3-dichloro-6-hydroxyphenyl)-hexahydro-1H-indolizin-5-one) as an off-white solid (41.0 mg, 82%): LCMS (ESI) calc'd for C₁₄H₁₅Cl₂NO₂ [M+H]⁺: 300, 302 (3:2) found 300, 302 (3:2); ¹H NMR (400 MHz, CD₃OD) δ 7.24 (d, J=8.8 Hz, 1H), 6.75 (d, J=8.8 Hz, 1H), 4.29-4.17 (m, 1H), 4.17-4.09 (m, 1H), 3.77-3.65 (m, 1H), 3.56-3.52 (m, 1H), 2.48-2.26 (m, 3H), 2.23-2.09 (m, 2H), 2.05-1.94 (m, 1H), 1.88-1.72 (m, 1H), 1.52-1.37 (m, 1H).

### Example 76. Compound 224 ((7R,8S)-rel-(2R, 8aS)-2-(2,3-dichloro-6-hydroxyphenyl)-7,8-dihydroxy-hexahydro-1H-indolizin-5-one) and Compound 225 ((7S,8R)-rel-(2R, 8aS)-2-(2,3-dichloro-6-hydroxyphenyl)-7,8-dihydroxy-hexahydro-1H-indolizin-5-one)

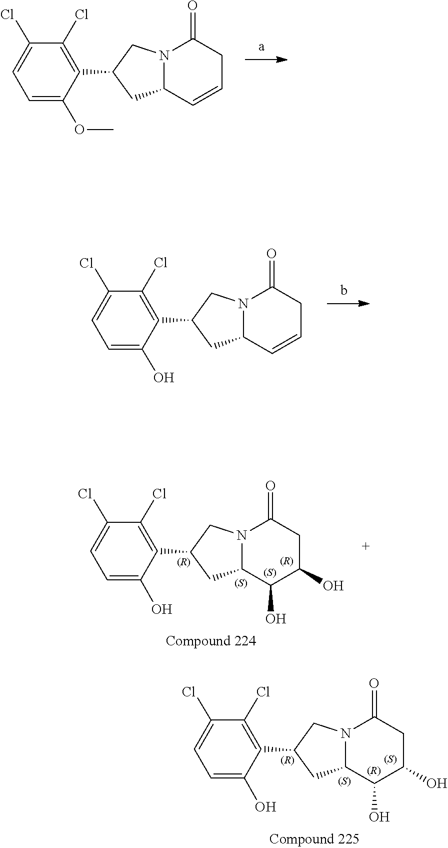

Step a:

To a stirred solution of (2R,8aS)-2-(2,3-dichloro-6-methoxyphenyl)-2,3,6,8a-tetrahydro-1H-indolizin-5-one (Intermediate 16, Example 14) (70.0 mg, 0.22 mmol) in DCM (1 mL) was added BBr₃ (0.07 mL, 0.28 mmol) at room temperature. The reaction was stirred at room temperature for 2 h, quenched with MeOH (5 mL) and concentrated under reduced pressure. The residue was purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 37% B to 60% B in 5.5 min; Detector: UV 210 nm; Retention time: 5.56 min. The fractions containing the desired product were collected and concentrated under reduced pressure to afford (2R,8aS)-2-(2,3-dichloro-6-hydroxyphenyl)-2,3,6,8a-tetrahydro-1H-indolizin-5-one as an off-white solid (20.0 mg, 28%): LCMS (ESI) calc'd for C₁₄H₁₃Cl₂NO₂ [M+H]⁺: 298, 300 (3:2) found 298, 300 (3:2); ¹H NMR (400 MHz, CD₃OD) δ 7.25 (d, J=8.8 Hz, 1H), 6.75 (d, J=8.8 Hz, 1H), 5.99 (d, J=10.1 Hz, 1H), 5.92-5.83 (m, 1H), 4.42-4.28 (m, 2H), 4.28-4.19 (m, 1H), 3.55-3.51 (m, 1H), 3.10-2.98 (m, 1H), 2.95-2.90 (m, 1H), 2.40-2.35 (m, 1H), 2.27-2.19 (m, 1H).

Step b:

To a stirred solution of (2R,8aS)-2-(2,3-dichloro-6-hydroxyphenyl)-2,3,6,8a-tetrahydro-1H-indolizin-5-one (0.200 g, 0.67 mmol) in THE (1 mL), acetone (1 mL) and H₂O (1 mL) were added NMO (0.120 g, 1.01 mmol) and K₂OsO₄ 2H₂O (49.0 mg, 0.13 mmol) at room temperature. The reaction was stirred at room temperature for 2 h and quenched with saturated aq. Na₂S₂O₃ (10 mL) followed by extraction with EA (3×20 mL). The combined organic layers were washed with brine (3×20 mL) and dried over anhydrous Na₂SO₄. After filtration, the filtrate was concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 33% B to 50% B in 5.5 min; Detector: UV 210 nm; Retention time 1: 5.53 min; Retention time 2: 6.12 min. The faster-eluting isomer at 5.53 min was obtained as Compound 224 ((7R,8S)-rel-(2R,8aS)-2-(2,3-dichloro-6-hydroxyphenyl)-7,8-dihydroxy-hexahydro-1H-indolizin-5-one) as an off-white solid (2.40 mg, 1.08%): LCMS (ESI) calc'd for C₁₄H₁₅Cl₂NO₄ [M+H]⁺: 332, 334 (3:2) found 332, 334 (3:2); ¹H NMR (400 MHz, CD₃OD) δ 7.25 (d, J=8.8 Hz, 1H), 6.76 (d, J=8.8 Hz, 1H), 4.33-4.19 (m, 1H), 4.13-4.03 (m, 2H), 3.95-3.84 (m, 1H), 3.67 (dd, J=9.3, 2.3 Hz, 1H), 3.56-3.50 (m, 1H), 2.70 (dd, J=18.4, 4.3 Hz, 1H), 2.56-2.41 (m, 2H), 2.36-2.24 (m, 1H). The slower-eluting isomer at 6.12 min was obtained as Compound 225 ((7S,8R)-rel-(2R,8aS)-2-(2,3-dichloro-6-hydroxyphenyl)-7,8-dihydroxy-hexahydro-1H-indolizin-5-one) as off-white solid (31.9 mg, 14.32%): LCMS (ESI) calc'd for C₁₄H₁₅Cl₂NO₄ [M+H]⁺: 332, 334 (3:2) found 332, 334 (3:2); ¹H NMR (400 MHz, CD₃OD) δ 7.24 (d, J=8.8 Hz, 1H), 6.75 (d, J=8.8 Hz, 1H), 4.32-4.17 (m, 1H), 4.11-4.00 (m, 3H), 3.86-3.74 (m, 1H), 3.48 (dd, J=11.3, 9.5 Hz, 1H), 3.03-2.97 (m, 1H), 2.64-2.44 (m, 2H), 1.91-1.82 (m, 1H).

Example 77. Compound 226 was prepared in an analogous fashion as that described for compounds 224 and 225.

| Compound | | | |
|---|---|---|---|
| Number | Structure | Chemical Name | MS: (M + H)⁺ & ¹H MNR |
| 226 | 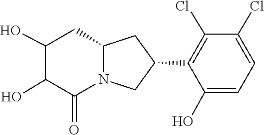 | (2R,8aS)-2-(2,3- dichloro-6- hydroxyphenyl)-6,7- dihydroxy- hexahydro-1H- indolizin-5-one | [M + H]⁺: 332, 334 (3:2); ¹H NMR (400 MHz, CD₃OD) δ 7.24 (d, J = 8.8 Hz, 1H), 6.75 (d, J = 8.8 Hz, 1H), 4.37-4.26 (m, 2H), 4.13-4.00 (m, 3H), 3.59-3.49 (m, 1H), 2.39-2.25 (m, 2H), 2.17-2.03 (m, 1H), 1.85-1.78 (m, 1H). |

### Example 78. Compound 227 ((2R,8aS)-2-(2,3-dichloro-6-hydroxyphenyl)-8-hydroxy-hexahydro-1H-indolizin-5-one isomer 1)

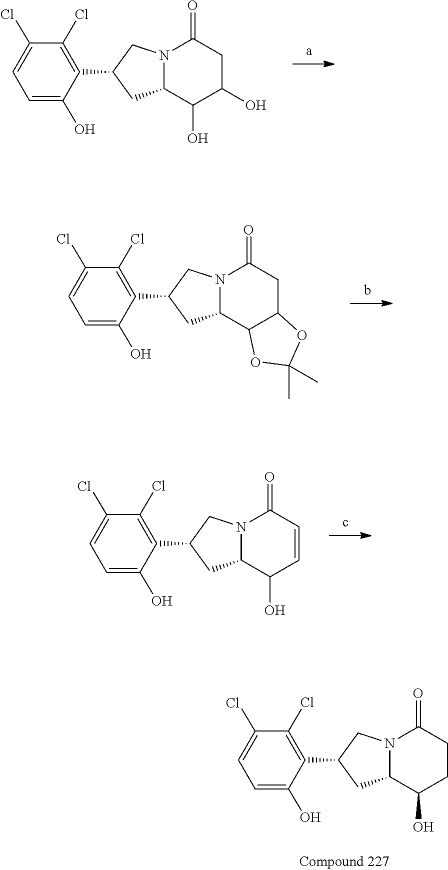 [SMILES: O=C1CC(O)C(O)[C@@H]2C[C@H](c3c(O)ccc(Cl)c3Cl)CN12 ; CC1(C)OC2CC(=O)N3C[C@@H](c4c(O)ccc(Cl)c4Cl)C[C@H]3C2O1 ; O=C1C=CC(O)[C@@H]2C[C@H](c3c(O)ccc(Cl)c3Cl)CN12 ; O=C1CC[C@@H](O)[C@@H]2C[C@H](c3c(O)ccc(Cl)c3Cl)CN12]

Step a:

To a stirred mixture of (2R,8aS)-2-(2,3-dichloro-6-hydroxyphenyl)-7,8-dihydroxy-hexahydro-1H-indolizin-5-one (0.100 g, 0.30 mmol) and TsOH (5.00 mg, 0.03 mmol) in acetone (3 mL) was added 2,2-dimethoxypropane (63.0 mg, 0.60 mmol) at room temperature. The reaction was stirred at room temperature for 2 h and quenched with saturated aq. NaHCO₃ (5 mL) followed by extraction with EA (3×10 mL). The combined organic layers were washed with brine (3×10 mL) and dried over anhydrous Na₂SO₄. After filtration, the filtrate was concentrated under reduced pressure to afford (8R,9aS)-8-(2,3-dichloro-6-hydroxyphenyl)-2,2-dimethyl-hexahydro-3aH-[1,3]dioxolo[4,5-g]indolizin-5-one as an off-white solid (0.100 g, crude), which was used in the next step directly without purification: LCMS (ESI) calc'd for C₁₇H₁₉Cl₂NO₄ [M+H]⁺: 372, 374 (3:2) found 372, 374 (3:2); ¹H NMR (400 MHz, CDCl₃) δ 9.00 (s, 1H), 7.19 (d, J=8.8 Hz, 1H), 6.86 (d, J=8.8 Hz, 1H), 4.79-4.71 (m, 1H), 4.49 (dd, J=7.5, 2.7 Hz, 1H), 4.36-4.24 (m, 1H), 4.10-3.96 (m, 1H), 3.77-3.63 (m, 1H), 3.58-3.46 (m, 1H), 2.92-2.81 (m, 1H), 2.77 (dd, J=15.5, 2.2 Hz, 1H), 2.41 (dd, J=15.4, 3.7 Hz, 1H), 2.22-2.09 (m, 1H), 1.38 (d, J=15.5 Hz, 6H).

Step b:

To a stirred solution of (8R,9aS)-8-(2,3-dichloro-6-hydroxyphenyl)-2,2-dimethyl-hexahydro-3aH-[1,3]dioxolo[4,5-g]indolizin-5-one (60.0 mg, 0.16 mmol) in DMF (1 mL) was added Cs₂CO₃ (0.160 g, 0.48 mmol) at room temperature. The reaction was stirred at 80° C. for 3 h. After filtration, the filtrate was concentrated under reduced pressure, the residue was purified by reverse phase chromatography, eluting with 30% ACN in water (plus 10 mM NH₄HCO₃) to afford (2R,8aS)-2-(2,3-dichloro-6-hydroxyphenyl)-8-hydroxy-2,3,8,8a-tetrahydro-1H-indolizin-5-one as a yellow solid (20.0 mg, 33%): LCMS (ESI) calc'd for C₁₄H₁₃Cl₂NO₃ [M+H]⁺: 314, 316 (3:2) found 314, 316 (3:2); ¹H NMR (400 MHz, CDCl₃) δ 7.23 (d, J=8.6 Hz, 1H), 6.91 (d, J=7.8 Hz, 1H), 6.77 (d, J=8.9 Hz, 1H), 6.22 (d, J=9.6 Hz, 1H), 4.31-4.19 (m, 2H), 4.17-4.10 (m, 1H), 3.97-3.89 (m, 1H), 3.79-3.70 (m, 1H), 3.05-3.00 (m, 1H), 2.22-2.09 (m, 1H).

Step c:

To a stirred solution of (2R,8aS)-2-(2,3-dichloro-6-hydroxyphenyl)-8-hydroxy-2,3,8,8a-tetrahydro-1H-indolizin-5-one (20.0 mg, 0.06 mmol) in MeOH (1.00 mL) was added PtO₂ (3.00 mg, 0.01 mmol) at room temperature. The resulting mixture was stirred for 1 h at room temperature under hydrogen atmosphere (1.5 atm). The resulting mixture was filtered and the filter cake was washed with MeOH (3×3 mL). The filtrate was concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 35% B to 40% B in 6.5 min; Detector: UV 254/210 nm; Retention time: 6.54 min. The fractions containing the desired product were collected and concentrated under reduced pressure to afford Compound 227 ((2R,8aS)-2-(2,3-dichloro-6-hydroxyphenyl)-8-hydroxy-hexahydro-1H-indolizin-5-one isomer 1) as a dark yellow solid (5.00 mg, 24%): LCMS (ESI) calc'd for C₁₄H₁₅Cl₂NO₃ [M+H]⁺: 316, 318 (3:2) found 316, 318 (3:2); ¹H NMR (400 MHz, CD₃OD) δ 7.24 (d, J=8.8 Hz, 1H), 6.75 (d, J=8.8 Hz, 1H), 4.28-4.16 (m, 1H), 4.16-4.07 (m, 2H), 3.81 (dd, J=12.1, 5.2 Hz, 1H), 3.56-3.48 (m, 1H), 2.96-2.90 (m, 1H), 2.62-2.48 (m, 1H), 2.34 (dd, J=18.0, 7.1 Hz, 1H), 2.14-2.04 (m, 1H), 2.04-1.93 (m, 1H), 1.93-1.84 (m, 1H).

### Example 79. Compound 228 ((2R,8aS)-2-(2,3-dichloro-6-hydroxyphenyl)-5-oxo-octahydroindolizine-7-carbonitrile isomer 1)

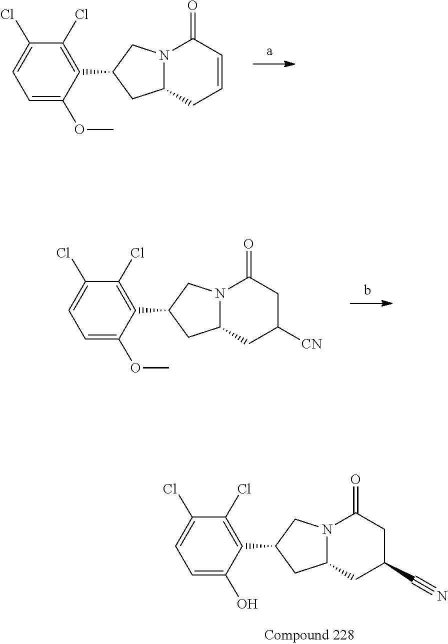

Step a:

To a stirred solution of (2R,8aR)-2-(2,3-dichloro-6-methoxyphenyl)-2,3,8,8a-tetrahydro-1H-indolizin-5-one (Intermediate 17, Example 15) (0.600 g, 1.92 mmol) and ZnI₂ (61.0 mg, 0.19 mmol) in DCE (6 mL) was added TMSCN (0.570 g, 5.77 mmol) at room temperature. The resulting mixture was stirred at 80° C. for 16 h and quenched with saturated aq. NaHCO₃ (20 mL) followed by extraction with EA (3×20 mL). The combined organic layers were washed with brine (3×20 mL) and dried over anhydrous Na₂SO₄. After filtration, the filtrate was concentrated under reduced pressure. The residue was purified by silica gel column chromatography, eluting with EA to afford (2R,8aS)-2-(2,3-dichloro-6-methoxyphenyl)-5-oxo-hexahydro-1H-indolizine-7-carbonitrile as an off-white solid (0.450 g, 62%): LCMS (ESI) calc'd for C₁₆H₁₆Cl₂N₂O₂ [M+MeCN+H]⁺: 380, 382 (3:2) found 380, 382 (3:2); ¹H NMR (400 MHz, CD3Cl) δ 7.36 (d, J=8.9 Hz, 1H), 6.77 (d, J=9.0 Hz, 1H), 4.33-4.29 (m, 1H), 4.16-4.02 (m, 2H), 3.82 (s, 3H), 3.68-3.59 (m, 1H), 3.36-3.30 (m, 1H), 2.82 (d, J=17.9 Hz, 1H), 2.69 (dd, J=18.1, 7.4 Hz, 1H), 2.50-2.42 (m, 1H), 2.29-2.21 (m, 2H), 1.78-1.68 (m, 1H).

Step b:

To a stirred solution/mixture of (2R,8aS)-2-(2,3-dichloro-6-methoxyphenyl)-5-oxo-hexahydro-1H-indolizine-7-carbonitrile (60.0 mg, 0.18 mmol) in DCM (2 mL) was added BBr₃ (0.130 g, 0.53 mmol) at room temperature. The reaction was stirred at room temperature for 4 h, quenched with water (3 mL) at 0° C. and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 20% B to 50% B in 5.5 min; Detector: UV 210 nm; Retention time: 5.65 min. The fractions containing the desired product were collected and concentrated under reduced pressure to afford Compound 228 ((2R,8aS)-2-(2,3-dichloro-6-hydroxyphenyl)-5-oxo-octahydroindolizine-7-carbonitrile isomer 1) as an off-white solid (9.00 mg, 16%): LCMS (ESI) calc'd for C₁₅H₁₄Cl₂N₂O₂ [M+H]⁺: 325, 327 (3:2) found 325, 327 (3:2); ¹H NMR (400 MHz, CD₃OD) δ 7.26 (d, J=8.8 Hz, 1H), 6.76 (d, J=8.8 Hz, 1H), 4.39-4.26 (m, 1H), 4.17 (dd, J=11.7, 9.0 Hz, 1H), 4.10-3.99 (m, 1H), 3.65-3.54 (m, 1H), 3.54-3.47 (m, 1H), 2.76 (dd, J=18.2, 7.1 Hz, 1H), 2.66 (d, J=18.1 Hz, 1H), 2.50-2.36 (m, 2H), 2.30-2.21 (m, 1H), 1.85-1.75 (m, 1H).

### Example 80. Compound 229 ((2R,8aS)-2-(2,3-dichloro-6-hydroxyphenyl)-5-oxo-hexahydro-1H-indolizine-7-carboxamide)

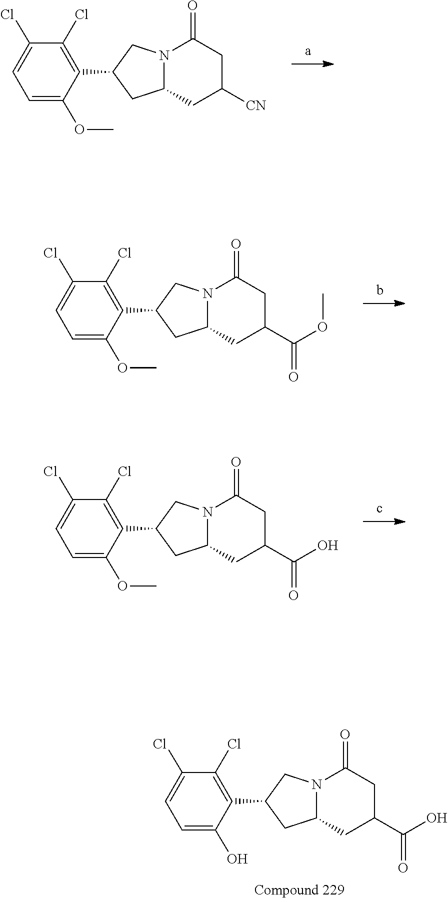

Step a:

To a stirred solution of (2R,8aS)-2-(2,3-dichloro-6-methoxyphenyl)-5-oxo-hexahydro-1H-indolizine-7-carbonitrile (Example 79, step a) (0.500 g, 1.47 mmol) in MeOH (5 mL) was added SOCl₂ (5 mL) dropwise at 0° C. The reaction was stirred at room temperature for 1 h and quenched with water (1 mL) and saturated aq. NaHCO₃ (1 mL) followed by extraction with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford methyl (2R,8aS)-2-(2,3-dichloro-6-methoxyphenyl)-5-oxo-hexahydro-1H-indolizine-7-carboxylate as a yellow solid (0.250 g, 46%): LCMS (ESI) calc'd for C₁₇H₁₉Cl₂NO₄ [M+H]⁺ 372, 374 (3:2) found 372, 374 (3:2); ¹H NMR (400 MHz, CDCl₃) δ 7.34 (dd, J=8.9, 1.2 Hz, 1H), 6.77 (dd, J=8.9, 1.5 Hz, 1H), 4.26-4.05 (m, 2H), 3.82 (d, J=1.0 Hz, 3H), 3.77 (d, J=3.7 Hz, 3H), 3.75-3.64 (m, 1H), 3.62-3.53 (m, 1H), 3.15-3.06 (m, 1H), 2.94 (d, J=17.9 Hz, 1H), 2.68-2.46 (m, 2H), 2.24-2.14 (m, 2H), 1.79-1.67 (m, 1H).

Step b:

To a stirred solution of methyl (2R,8aS)-2-(2,3-dichloro-6-methoxyphenyl)-5-oxo-hexahydro-1H-indolizine-7-carboxylate (90.0 mg, 0.24 mmol) in MeOH (2 mL) and H₂O (1 mL) was added LiOH H₂O (51.0 mg, 1.21 mmol) at room temperature. The reaction was stirred at 40° C. for 1 h, acidified with aq. HCl (10%) to pH 4 followed by extraction with DCM (3×30 mL). The combined organic layers were dried over anhydrous Na₂SO₄. After filtration, the filtrate was concentrated under reduced pressure to afford (2R,8aS)-2-(2,3-dichloro-6-methoxyphenyl)-5-oxo-hexahydro-1H-indolizine-7-carboxylic acid as light yellow solid (70.0 mg, crude), which was used in the next step directly without purification: LCMS (ESI) calc'd for C₁₆H₁₇Cl₂NO₄ [M+H]⁺ 358, 360 (3:2) found 358, 360 (3:2); ¹H NMR (400 MHz, CD₃OD) δ 7.42 (dd, J=8.9, 2.3 Hz, 1H), 7.00 (dd, J=9.0, 2.2 Hz, 1H), 4.36-4.22 (m, 1H), 4.03 (dd, J=11.9, 8.2 Hz, 1H), 3.85 (d, J=2.5 Hz, 3H), 3.78-3.67 (m, 1H), 3.55-3.44 (m, 1H), 3.16-3.08 (m, 1H), 2.83-2.74 (m, 1H), 2.59-2.47 (m, 2H), 2.29-2.10 (m, 2H), 1.85-1.71 (m, 1H).

Step c:

To a stirred solution of (2R,8aS)-2-(2,3-dichloro-6-methoxyphenyl)-5-oxo-hexahydro-1H-indolizine-7-carboxylic acid (40.0 mg, 0.11 mmol) in DCM (2 mL was added BBr₃ (0.280 g, 1.12 mmol) at room temperature. The reaction was stirred at room temperature for 1 h, quenched with H₂O (2 mL) and evaporated under reduced pressure. The residue was purified by reverse phase chromatography, eluting with 35% ACN in water (plus 0.05% TFA) to afford the crude product, which was then purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 35% B to 60% B in 6.5 min; Detector: UV 210 nm; Retention time: 6.54 min. The fractions containing the desired product were collected and concentrated under reduced pressure to afford Compound 229 (2R,8aS)-2-(2,3-dichloro-6-hydroxyphenyl)-5-oxo-hexahydro-1H-indolizine-7-carboxylic acid as an off-white solid (20.0 mg, 52%): LCMS (ESI) calc'd for C₁₅H₁₅Cl₂NO₄ [M+H]⁺ 344, 346 (3:2) found 344, 346 (3:2); ¹H NMR (400 MHz, CD₃OD) δ 7.25 (d, J=8.8 Hz, 1H), 6.77 (d, J=8.7 Hz, 1H), 4.35-4.02 (m, 2H), 3.88-3.72 (m, 1H), 3.62-3.45 (m, 1H), 3.03-2.88 (m, 1H), 2.74-2.58 (m, 1H), 2.58-2.33 (m, 3H), 2.23-2.00 (m, 1H), 1.58-1.50 (m, 1H).

### Example 81. Compound 230 ((2R,8aS)-2-(2,3-dichloro-6-hydroxyphenyl)-5-oxo-hexahydro-1H-indolizine-7-carboxamide isomer 1) and Compound 231 ((2R,8aS)-2-(2,3-dichloro-6-hydroxyphenyl)-5-oxo-hexahydro-1H-indolizine-7-carboxamide isomer 2)

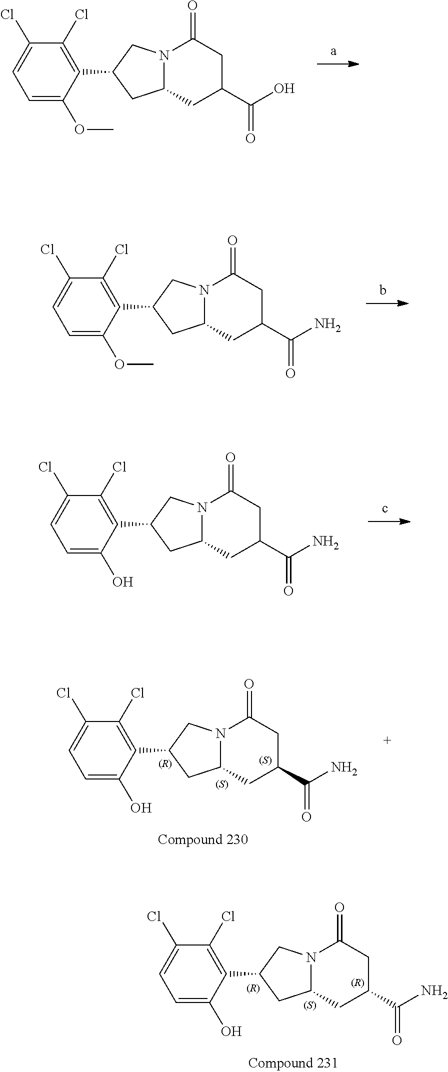

Step a:

To a stirred solution of (2R,8aS)-2-(2,3-dichloro-6-methoxyphenyl)-5-oxo-hexahydro-1H-indolizine-7-carboxylic acid (Example 80, step b) (50.0 mg, 0.14 mmol) and HATU (0.110 g, 0.28 mmol) in DMF (3 mL) was added NH₄Cl (37.0 mg, 0.70 mmol) at room temperature. The reaction was stirred at room temperature for 1 h and diluted with water (20 mL) followed by extraction with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 43% ACN in water (plus 0.1% FA) to afford (2R,8aS)-2-(2,3-dichloro-6-methoxyphenyl)-5-oxo-hexahydro-1H-indolizine-7-carboxamide as an off-white solid (30.0 mg, 60%): LCMS (ESI) calc'd for C₁₆H₁₈Cl₂N₂O₃ [M+H]⁺ 357, 359 (3:2) found 357, 359 (3:2);

Step b:

To a stirred solution of (2R,8aS)-2-(2,3-dichloro-6-methoxyphenyl)-5-oxo-hexahydro-1H-indolizine-7-carboxamide (35.0 mg, 0.10 mmol) in DCM (2 mL) was added BBr₃ (0.150 g, 0.59 mmol) at room temperature. The reaction was stirred at room temperature for 1 h, quenched with MeOH (2 mL) and concentrated under reduced pressure. The residue was purified by reverse phase chromatography, eluting with 40% ACN in water (plus 0.1% FA) to afford the crude product, which was then purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 15% B to 35% B in 5.5 min; Detector: UV 210 nm; Retention time: 5.6 min. The fractions containing the desired product were collected and concentrated under reduced pressure to afford (2R,8aS)-2-(2,3-dichloro-6-hydroxyphenyl)-5-oxo-hexahydro-1H-indolizine-7-carboxamide as an off-white solid (17.5 mg, 52%): LCMS (ESI) calc'd for C₁₅H₁₆Cl₂N₂O₃ [M+H]⁺ 343, 345 (3:2) found 343, 345 (3:2); ¹H NMR (400 MHz, CD₃OD) δ 7.25 (d, J=8.8 Hz, 1H), 6.77 (d, J=8.7 Hz, 1H), 4.34-4.05 (m, 2H), 3.89-3.74 (m, 1H), 3.59-3.47 (m, 1H), 2.98-2.79 (m, 1H), 2.60-2.51 (m, 2H), 2.46-2.12 (m, 3H), 1.68-1.52 (m, 1H).

Step c:

(2R,8aS)-2-(2,3-dichloro-6-hydroxyphenyl)-5-oxo-hexahydro-1H-indolizine-7-carboxamide (15.0 mg, 0.04 mmol) was separated with Prep Chiral HPLC with the following conditions: Column: CHIRALPAK IG, 20×250 mm, 5 μm; Mobile Phase A: Hex (plus 0.1% FA)-HPLC, Mobile Phase B: EtOH-HPLC; Flow rate: 20 mL/min; Gradient: 50% B to 50% B in 16 min; Detector: UV 224 nm; Retention Time 1: 5.00 min; Retention Time 2: 11.91 min. The faster-eluting isomer at 5.00 min was obtained as Compound 230 ((2R,8aS)-2-(2,3-dichloro-6-hydroxyphenyl)-5-oxo-hexahydro-1H-indolizine-7-carboxamide isomer 1) as an off-white solid (6.00 mg, 40.00%): LCMS (ESI) calc'd for C₁₅H₁₆Cl₂N₂O₃ [M+H]⁺ 343, 345 (3:2) found 343, 345 (3:2); ¹H NMR (400 MHz, CD₃OD) δ 7.25 (d, J=8.8 Hz, 1H), 6.78 (d, J=8.8 Hz, 1H), 4.26-4.05 (m, 3H), 3.68-3.52 (m, 1H), 2.92-2.81 (m, 1H), 2.55 (d, J=8.7 Hz, 2H), 2.48-2.38 (m, 1H), 2.32-2.24 (m, 1H), 2.14-2.03 (m, 1H), 1.63-1.55 (m, 1H). The slower-eluting isomer at 11.91 min was obtained as Compound 231 ((2R,8aS)-2-(2,3-dichloro-6-hydroxyphenyl)-5-oxo-hexahydro-1H-indolizine-7-carboxamide isomer 1) as an off-white solid (1.40 mg, 9.33%): LCMS (ESI) calc'd for C₁₅H₁₆Cl₂N₂O₃ [M+H]⁺ 343, 345 (3:2) found 343, 345 (3:2); ¹H NMR (400 MHz, CD₃OD) δ 7.25 (d, J=8.8 Hz, 1H), 6.75 (d, J=8.8 Hz, 1H), 4.33-4.16 (m, 1H), 4.16-4.07 (m, 1H), 3.86-3.72 (m, 1H), 3.56-3.45 (m, 1H), 2.94-2.80 (m, 1H), 2.56 (d, J=8.3 Hz, 2H), 2.44-2.28 (m, 2H), 2.24-2.12 (m, 1H), 1.65-1.55 (m, 1H).

Example 81. Compounds 232-236 are prepared in an analogous fashion as that described for Compounds 230-231.

| Compound | | | |
|---|---|---|---|
| Number | Structure | Chemical Name | MS: (M + H)⁺ & ¹H MNR |
| 232 | 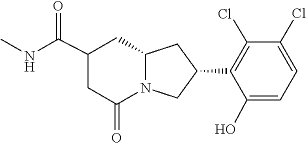 | (2R,8aS)-2-(2,3- dichloro-6- hydroxyphenyl)-N- methyl-5-oxo- hexahydro-1H- indolizine-7- carboxamide | [M + H]⁺: 357, 359 (3:2); ¹H NMR (400 MHz, CD₃OD) δ 7.24 (d, J = 8.8 Hz, 1H), 6.75 (d, J = 8.8 Hz, 1H), 4.27-4.17 (m, 1H), 4.13-4.09 (m, 1H), 3.78-3.69 (m, 1H), 3.58-3.48 (m, 1H), 3.03- 2.97 (m, 1H), 2.76 (s, 3H), 2.65- 2.60 (m, 1H), 2.49-2.28 (m, 3H), 2.22-2.09 (m, 1H), 1.85-1.75 (m, |
| | | | 1H). |
| 233 | 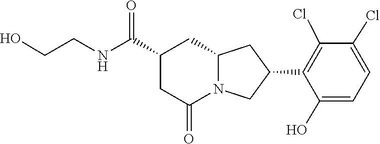 [SMILES: O=C(NCCO)[C@H]1CC(=O)N2C[C@@H](c3c(O)ccc(Cl)c3Cl)C[C@H]2C1] | (2R,8aS)-2-(2,3- dichloro-6- hydroxyphenyl)-N- (2-hydroxyethyl)- 5-oxo-hexahydro- 1H-indolizine-7- carboxamide isomer 1 | [M + H]⁺: 387, 389 (3:2); ¹H NMR (400 MHz, CD₃OD) δ 7.25 (d, J = 8.8 Hz, 1H), 6.75 (d, J = 8.8 Hz, 1H), 4.32-4.21 (m, 1H), 4.13 (dd, J = 11.6, 9.0 Hz, 1H), 3.84-3.74 (m, 1H), 3.65-3.61 (m, 2H), 3.58-3.48 (m, 1H), 3.37- 3.33 (m, 2H), 2.92-2.79 (m, 1H), 2.64-2.53 (m, 2H), 2.45-2.36 (m, |
| | | | 1H), 2.33-2.26 (m, 1H), 2.22- |
| | | | 2.16 (m, 1H), 1.67-1.58 (m, 1H). |
| 234 | 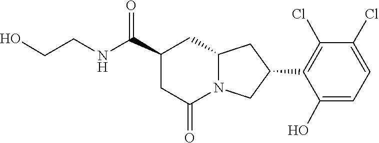 [SMILES: O=C(NCCO)[C@@H]1CC(=O)N2C[C@@H](c3c(O)ccc(Cl)c3Cl)C[C@H]2C1] | (2R,8aS)-2-(2,3- dichloro-6- hydroxyphenyl)-N- (2-hydroxyethyl)- 5-oxo-hexahydro- 1H-indolizine-7- carboxamide isomer 2 | [M + H]⁺: 387, 389 (3:2); ¹H NMR (400 MHz, CD₃OD) δ 7.25 (d, J = 8.8 Hz, 1H), 6.75 (d, J = 8.8 Hz, 1H), 4.32-4.21 (m, 1H), 4.13 (dd, J = 11.6, 9.0 Hz, 1H), 3.84-3.75 (m, 1H), 3.65-3.62 (m, 2H), 3.58-3.48 (m, 1H), 3.37- 3.33 (m, 2H), 2.92-2.79 (m, 1H), 2.64-2.53 (m, 2H), 2.45-2.36 (m, |
| | | | 1H), 2.33-2.26 (m, 1H), 2.22- |
| | | | 2.16 (m, 1H), 1.67-1.57 (m, 1H). |
| 235 | 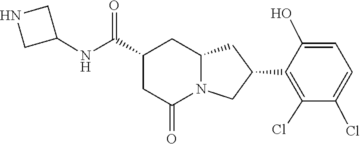 [SMILES: O=C(NC1CNC1)[C@H]1CC(=O)N2C[C@@H](c3c(O)ccc(Cl)c3Cl)C[C@H]2C1] | (2R,8aS)-N- (azetidin-3-yl)-2- (2,3-dichloro-6- hydroxyphenyl)-5- oxo-hexahydro- 1H-indolizine-7- carboxamide isomer 1 | [M + H]⁺: 398, 400 (3:2); ¹H NMR (400 MHz, CD₃OD) δ 7.26 (d, J = 8.8 Hz, 1H), 6.76 (d, J = 8.8 Hz, 1H), 4.69-4.61 (m, 1H), 4.36-4.09 (m, 6H), 3.85-3.76 (m, 1H), 3.59-3.49 (m, 1H), 2.92- 2.80 (m, 1H), 2.57 (d, J = 8.8 Hz, 2H), 2.45-2.29 (m, 2H), 2.24- 2.17 (m, 1H), 1.68-1.57 (m, 1H). |
| 236 | 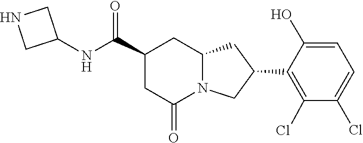 [SMILES: O=C(NC1CNC1)[C@@H]1CC(=O)N2C[C@@H](c3c(O)ccc(Cl)c3Cl)C[C@H]2C1] | (2R,8aS)-N- (azetidin-3-yl)-2- (2,3-dichloro-6- hydroxyphenyl)-5- oxo-hexahydro- 1H-indolizine-7- carboxamide isomer 2 | [M + H]⁺: 398, 400 (3:2); ¹H NMR (400 MHz, CD₃OD) δ 7.25 (d, J = 8.8 Hz, 1H), 6.76 (d, J = 8.8 Hz, 1H), 4.68-4.59 (m, 1H), 4.32 (dd, J = 11.1, 8.5 Hz, 2H), 4.28-4.17 (m, 3H), 4.20-4.06 (m, 1H), 3.77-3.68 (m, 1H), 3.55- 3.49 (m, 1H), 3.08-3.02 (m, 1H), 2.66-2.62 (m, 1H), 2.46 (dd, J = |
| | | | 17.7, 6.8 Hz, 2H), 2.45-2.36 (m, |
| | | | 1H), 2.18-2.11 (m, 1H), 1.88- |
| | | | 1.80 (m, 1H). |

### Example 82. Compound 237 ((2R,8a8)-2-(2,3-dichloro-6-hydroxyphenyl)-7-(hydroxymethyl)hexahydroindolizin-5(1H)-one isomer 1) and Compound 238 ((2R,8aS)-2-(2,3-dichloro-6-hydroxyphenyl)-7-(hydroxymethyl)hexahydroindolizin-5(1H)-one isomer 2)

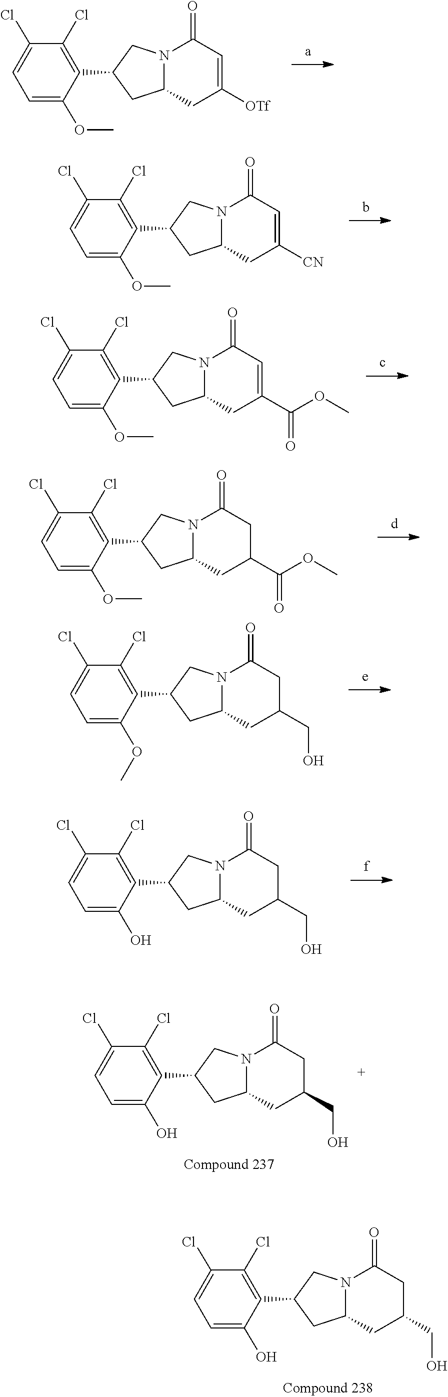

Step a:

To a stirred solution of (2R,8aS)-2-(2,3-dichloro-6-methoxyphenyl)-5-oxo-2,3,8,8a-tetrahydro-1H-indolizin-7-yl trifluoromethanesulfonate (0.260 g, 0.565 mmol) and Zn(CN)₂ (66.0 mg, 0.57 mmol) in DMF (4 mL) was added Pd(PPh₃)₄ (65.0 mg, 0.06 mmol) at room temperature under nitrogen atmosphere. The reaction was stirred 90° C. for 16 h, cooled to room temperature and diluted with NaHCO₃ (20 mL), followed by extraction with EA (3×30 mL). The combined organic layers were washed with brine (3×20 mL) and dried over anhydrous Na₂SO₄. After filtration, the filtrate was concentrated under reduced pressure. The residue was purified by silica gel column chromatography, eluting with PE/EA (2/1) to afford (2R,8aS)-2-(2,3-dichloro-6-methoxyphenyl)-5-oxo-2,3,8,8a-tetrahydro-1H-indolizine-7-carbonitrile as an off-white solid (0.150 g, 79%): LCMS (ESI) calc'd for C₁₆H₁₄Cl₂N₂O₂ [M+H]⁺ 337, 339 (3:2) found 337, 339 (3:2); ¹H NMR (400 MHz, CD₃OD) δ 7.38 (dd, J=8.9, 4.0 Hz, 1H), 6.80 (d, J=9.0 Hz, 1H), 6.62 (dd, J=8.6, 3.0 Hz, 1H), 4.42-4.18 (m, 1H), 4.12-3.95 (m, 2H), 3.84 (d, J=5.1 Hz, 3H), 3.81-3.67 (m, 1H), 2.79-2.65 (m, 1H), 2.60-2.39 (m, 2H), 2.36-2.07 (m, 1H).

Step b:

To a stirred solution of (2R,8aS)-2-(2,3-dichloro-6-methoxyphenyl)-5-oxo-2,3,8,8a-tetrahydro-1H-indolizine-7-carbonitrile (65.0 mg, 0.19 mmol) in MeOH (2 mL) was added SOCl₂ (2 mL) at room temperature. The reaction was stirred at room temperature for 16 h, quenched with saturated aq. NaHCO₃ (20 mL) and extracted with EA (3×30 mL). The combined organic layers were washed with brine (3×20 mL) and dried over anhydrous Na₂SO₄. After filtration, the filtrate was concentrated under reduced pressure. The residue was purified by silica gel column chromatography, eluting with PE/EA (1/1) to afford methyl (2R,8aS)-2-(2,3-dichloro-6-methoxyphenyl)-5-oxo-2,3,8,8a-tetrahydro-1H-indolizine-7-carboxylate as a light yellow semisolid (50.0 mg, 70%): LCMS (ESI) calc'd for C₁₇H₁₇Cl₂NO₄ [M+H]⁺ 370, 372 (3:2) found 370, 372 (3:2); ¹H NMR (400 MHz, CD₃OD) δ 7.48-7.41 (m, 1H), 7.02 (d, J=9.0 Hz, 1H), 6.71 (d, J=3.0 Hz, 1H), 4.48-4.24 (m, 1H), 4.15-3.91 (m, 2H), 3.91-3.82 (m, 6H), 3.76-3.64 (m, 1H), 3.06 (dd, J=17.4, 5.0 Hz, 1H), 2.56-2.18 (m, 3H).

Step c:

To a stirred solution of methyl (2R,8aS)-2-(2,3-dichloro-6-methoxyphenyl)-5-oxo-2,3,8,8a-tetrahydro-1H-indolizine-7-carboxylate (50.0 mg, 0.14 mmol) in MeOH (1 mL) was added PtO₂ (31.0 mg, 0.14 mmol) at room temperature. The reaction was stirred for 1 h under hydrogen atmosphere (1.5 atm). The resulting mixture was filtered and the filtrate was concentrated under reduced pressure to afford methyl (2R,8aS)-2-(2,3-dichloro-6-methoxyphenyl)-5-oxo-hexahydro-1H-indolizine-7-carboxylate as a colorless oil (50.0 mg, 99%): LCMS (ESI) calc'd for C₁₇H₁₉Cl₂NO₄ [M+H]⁺ 372, 374 (3:2) found 372, 374 (3:2); ¹H NMR (400 MHz, CDCl₃) δ 7.35 (dd, J=8.9, 2.6 Hz, 1H), 6.76 (d, J=9.0 Hz, 1H), 4.21 (q, J=9.2 Hz, 1H), 4.14-4.05 (m, 1H), 3.82 (s, 3H), 3.76 (d, J=1.7 Hz, 3H), 3.73-3.66 (m, 1H), 3.61-3.51 (m, 1H), 2.96-2.83 (m, 1H), 2.82-2.72 (m, 1H), 2.67-2.54 (m, 1H), 2.50-2.39 (m, 1H), 2.26-2.15 (m, 2H), 1.68-1.61 (m, 1H).

Step d:

To a stirred solution of methyl (2R,8aS)-2-(2,3-dichloro-6-methoxyphenyl)-5-oxo-hexahydro-1H-indolizine-7-carboxylate (60.0 mg, 0.16 mmol) in MeOH (3 mL) was added NaBH₄ (0.180 g, 4.84 mmol) in portions at room temperature. The reaction was stirred for 16 h, quenched with saturated aq. NH₄Cl (20 mL) and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 34% ACN in water (plus 0.05% TFA) to afford (2R,8aS)-2-(2,3-dichloro-6-methoxyphenyl)-7-(hydroxymethyl)-hexahydro-1H-indolizin-5-one as a light yellow semisolid (40.0 mg, 72%): LCMS (ESI) calc'd for C₁₆H₁₉Cl₂NO₃ [M+H]⁺ 344, 346 (3:2) found 344, 346 (3:2); ¹H NMR (400 MHz, CD₃OD) δ 7.40 (d, J=9.0 Hz, 1H), 6.97 (d, J=9.1 Hz, 1H), 4.44-4.15 (m, 1H), 4.07-3.94 (m, 1H), 3.94-3.68 (m, 5H), 3.57-3.37 (m, 2H), 2.60-2.29 (m, 1H), 2.29-1.91 (m, 5H), 1.29-1.15 (m, 1H).

Step e:

To a stirred solution of (2R,8aS)-2-(2,3-dichloro-6-methoxyphenyl)-7-(hydroxymethyl)-hexahydro-1H-indolizin-5-one (35.0 mg, 0.10 mmol) in DCM (2 mL) was added BBr₃ (0.250 g, 1.02 mmol) at room temperature. The reaction was stirred at room temperature for 2 h, quenched with MeOH (1 mL) and concentrated under reduced pressure. The residue was purified by Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 38% B to 50% B in 5.5 min; Detector: UV 210 nm; Retention time: 5.56 min. The fractions containing the desired product were collected and concentrated under reduced pressure to afford (2R,8aS)-2-(2,3-dichloro-6-hydroxyphenyl)-7-(hydroxymethyl)-hexahydro-1H-indolizin-5-one as an off-white solid (15.7 mg, 46%): LCMS (ESI) calc'd for C₁₅H₁₇Cl₂NO₃ [M+H]⁺ 330, 332 (3:2) found 330, 332 (3:2); ¹H NMR (400 MHz, CD₃OD) δ 7.25 (dd, J=8.6, 1.8 Hz, 1H), 6.75 (dd, J=8.7, 1.9 Hz, 1H), 4.32-4.20 (m, 1H), 4.20-4.07 (m, 1H), 3.84-3.70 (m, 1H), 3.60-3.45 (m, 3H), 2.55-2.31 (m, 2H), 2.28-2.04 (m, 4H), 1.35-1.18 (m, 1H).

Step f:

The product (2R,8aS)-2-(2,3-dichloro-6-hydroxyphenyl)-7-(hydroxymethyl)-hexahydro-1H-indolizin-5-one (15.0 mg, 0.05 mmol) was separated by Prep Chiral HPLC with the following conditions: Column: CHIRALPAK ID-2, 2×25 cm, 5 μm; Mobile Phase A: Hex (plus 0.1% FA)-HPLC, Mobile Phase B: IPA-HPLC; Flow rate: 20 mL/min; Gradient: 20% to 20% in 24 min; Detector: UV 220/254 nm; Retention time 1: 8.61 min; Retention time 2: 14.51 min. The faster-eluting isomer at 8.61 min was obtained Compound 238 ((2R,8aS)-2-(2,3-dichloro-6-hydroxyphenyl)-7-(hydroxymethyl)-hexahydro-1H-indolizin-5-one isomer 1) as an off-white solid (1 mg, 6.67%): LCMS (ESI) calc'd for C₁₅H₁₇Cl₂NO₃ [M+H]⁺: 330, 332 (3:2) found 330, 332 (3:2); ¹H NMR (400 MHz, CD₃OD) δ 7.24 (d, J=8.7 Hz, 1H), 6.76 (d, J=8.8 Hz, 1H), 4.35-4.01 (m, 3H), 3.73-3.42 (m, 3H), 2.56-2.30 (m, 2H), 2.30-1.97 (m, 4H), 1.25-1.10 (m, 1H). The slower-eluting isomer at 14.51 min was obtained Compound 238 ((2R,8aS)-2-(2,3-dichloro-6-hydroxyphenyl)-7-(hydroxymethyl)-hexahydro-1H-indolizin-5-one isomer 2) as an off-white solid (5.6 mg, 37.33%): LCMS (ESI) calc'd for C₁₅H₁₇Cl₂NO₃ [M+H]⁺: 330, 332 (3:2) found 330, 332 (3:2); ¹H NMR (400 MHz, CD₃OD) δ 7.24 (d, J=8.7 Hz, 1H), 6.75 (d, J=8.8 Hz, 1H), 4.33-4.20 (m, 1H), 4.17-4.08 (m, 1H), 3.84-3.70 (m, 1H), 3.59-3.45 (m, 3H), 2.56-2.31 (m, 2H), 2.28-2.05 (m, 4H), 1.38-1.19 (m, 1H).

### Example 83. Compound 239 ((2R, 8aR)-2-(2,3-dichloro-6-hydroxyphenyl)-7-[(methylamino)methyl]-hexahydro-1H-indolizin-5-one isomer 1)

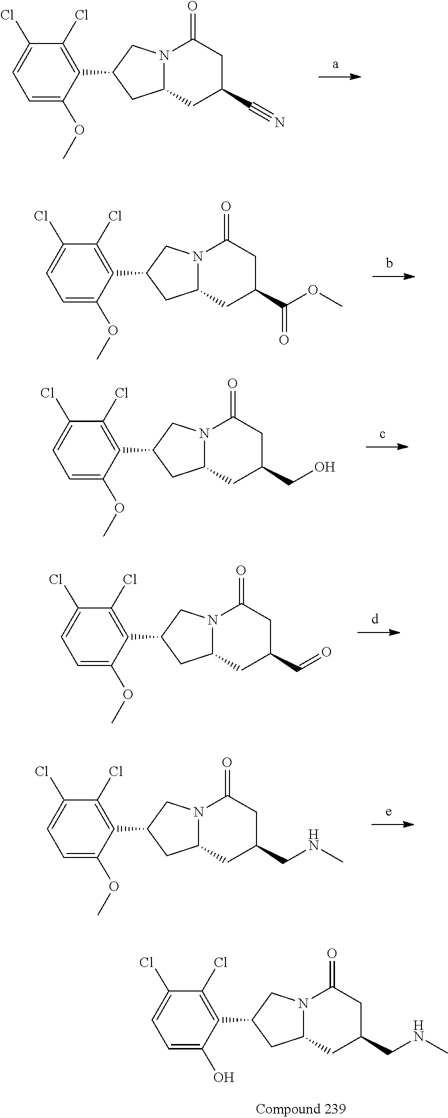

Step a:

To a stirred solution of (2R,8aS)-2-(2,3-dichloro-6-methoxyphenyl)-5-oxo-hexahydro-1H-indolizine-7-carbonitrile isomer 1 (0.400 g, 1.179 mmol) in MeOH (8 mL) was added SOCl₂ (4.00 mL, 13.785 mmol) dropwise at 0° C. under air atmosphere. The resulting mixture was stirred for 1 h, quenched with water (1 mL) and NaHCO₃ (1 mL) and extracted with EtOAc (3×10 mL). The combined organic layers were washed with brine (3×5 mL) and dried over anhydrous Na₂SO₄. After filtration, the filtrate was concentrated under reduced pressure. The residue was purified by reverse phase chromatography, eluted with 45% ACN in 0.05% TFA aqueous solution to afford methyl (2R,8aS)-2-(2,3-dichloro-6-methoxyphenyl)-5-oxo-hexahydro-1H-indolizine-7-carboxylate isomer 1 (250 mg, 56.96%) as a yellow solid. LCMS (ESI) calc'd for C₁₇H₁₉Cl₂NO₄ [M+H]⁺ 372, 374 (3:2) found 372, 374 (3:2); ¹H NMR (300 MHz, CDCl₃) δ 7.34 (d, J=8.9 Hz, 1H), 6.76 (d, J=8.9 Hz, 1H), 4.27-3.97 (m, 2H), 3.81 (s, 3H), 3.77 (s, 3H), 3.74-3.63 (m, 1H), 3.63-3.53 (m, 1H), 3.14-3.05 (m, 1H), 2.93 (d, J=17.7 Hz, 1H), 2.65-2.45 (m, 2H), 2.26-2.13 (m, 2H), 1.79-1.64 (m, 1H).

Step b:

To a stirred solution of methyl (2R,8aS)-2-(2,3-dichloro-6-methoxyphenyl)-5-oxo-hexahydro-1H-indolizine-7-carboxylate isomer 1 (0.200 g, 0.54 mmol) in MeOH (3 mL) was added NaBH₄ (41.0 mg, 1.07 mmol) at room temperature. The reaction was stirred at room temperature for 6 h, quenched with saturated aq. NH₄Cl (20 mL) followed by extraction with EA (2×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 0.05% TFA) to afford (2R,8aS)-2-(2,3-dichloro-6-methoxyphenyl)-7-(hydroxymethyl)-hexahydro-1H-indolizin-5-one isomer 1 as an off-white solid (0.150 g, 48%): LCMS (ESI) calc'd for C₁₆H₁₉Cl₂NO₃ [M+H]⁺: 344, 346 (3:2) found 344, 346 (3:2); ¹H NMR (300 MHz, CDCl₃) δ 7.35 (d, J=8.9 Hz, 1H), 6.77 (d, J=9.0 Hz, 1H), 4.30-4.08 (m, 3H), 3.82 (s, 3H), 3.70 (d, J=6.7 Hz, 2H), 3.63-3.51 (m, 1H), 2.65 (dd, J=17.6, 6.5 Hz, 1H), 2.43 (d, J=19.0 Hz, 1H), 2.38-2.10 (m, 4H), 1.74-1.59 (m, 1H).

Step c:

To a stirred solution of (2R,8aS)-2-(2,3-dichloro-6-methoxyphenyl)-7-(hydroxymethyl)-hexahydro-1H-indolizin-5-one isomer 1 (0.150 g, 0.20 mmol) in DCM (1 mL) was added Dess-Martin periodinane (0.350 g, 0.42 mmol) at room temperature. The reaction was stirred at room temperature for 2 h, quenched with saturated aq. Na₂S₂O₃ (20 mL) followed by extraction with EA (3×20 mL). The combined organic layers were washed with brine (3×20 mL) and dried over anhydrous Na₂SO₄. After filtration, the filtrate was concentrated under reduced pressure to afford (2R,8aS)-2-(2,3-dichloro-6-methoxyphenyl)-5-oxo-hexahydro-1H-indolizine-7-carbaldehyde isomer 1 as a yellow oil (0.130 g, crude), which was used in the next step directly without further purification: LCMS (ESI) calc'd for C₁₆H₁₇Cl₂NO₃ [M+H]⁺: 342, 344 (3:2) found 342, 344 (3:2).

Step d:

To a stirred solution of (2R,8aS)-2-(2,3-dichloro-6-methoxyphenyl)-5-oxo-hexahydro-1H-indolizine-7-carbaldehyde isomer 1 (0.130 g, 0.21 mmol) and methylamine (25.0 mg, 0.41 mmol) in DCM (1 mL) were added AcOH (24.0 mg, 0.20 mmol) and NaBH(AcO)₃ (0.260 g, 0.61 mmol) at room temperature. The reaction was for 2 h and quenched with saturated aq. Na₂S₂O₃ (20 mL) followed by extraction with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 40% ACN in water (plus 0.05% TFA) to afford (2R,8aR)-2-(2,3-dichloro-6-methoxyphenyl)-7-[(methylamino)methyl]-hexahydro-1H-indolizin-5-one isomer 1 as a yellow oil (20.0 mg, 16%): LCMS (ESI) calc'd for C₁₇H₂₂Cl₂N₂O₂ [M+H]⁺: 357, 359 (3:2) found 357, 359 (3:2): ¹H NMR (400 MHz, CDCl₃) δ 7.35 (d, J=8.9 Hz, 1H), 6.77 (d, J=8.9 Hz, 1H), 4.31-4.17 (m, 1H), 4.17-4.03 (m, 1H), 3.86-3.71 (m, 4H), 3.62-3.50 (m, 1H), 3.24-2.91 (m, 2H), 2.82 (s, 3H), 2.76-2.47 (m, 1H), 2.41-2.11 (m, 5H), 1.38-1.24 (m, 1H).

Step e:

To a stirred solution of (2R,8aR)-2-(2,3-dichloro-6-methoxyphenyl)-7-[(methylamino)methyl]-hexahydro-1H-indolizin-5-one isomer 1 (20.0 mg, 0.06 mmol) in DCM (1 mL) was added BBr₃ (0.05 mL, 0.53 mmol) at room temperature. The reaction was stirred for 1 h at room temperature and quenched with MeOH (1 mL). The residue was purified by Prep-HPLC with the following conditions: Column: X Select CSH Prep C18 OBD Column, 19×250 mm, 5 μm; Mobile Phase A: Water (plus 0.05% o TFA), Mobile Phase B: ACN; Flow rate: 20 mL/min; Gradient: 20% o to 4000 in 6.5 min; Detector: UV 254/220 nm; Retention time: 6.54 min. The fractions containing the desired product were collected and concentrated under reduced pressure to afford Compound 239 ((2R,8aR)-2-(2,3-dichloro-6-hydroxyphenyl)-7-[(methylamino)methyl]-hexahydro-1H-indolizin-5-one isomer 1) as a light yellow solid (6.10 mg, 320%): LCMS (ESI) calc'd for C₁₆H₂OCl₂N₂O₂ [M+H]⁺: 343, 345 (3:2) found 343, 345 (3:2); ¹H NMR (400 z, CD₃OD) δ 7.26 (d, Je 9.0 Hz, 1H), 6.76 (d, J=9.0 Hz, 1H), 4.35-4.22 (m, 1H), 4.22-4.07 (m, 1H), 3.90-3.73 (m, 1H), 3.60-3.45 (m, 1H), 3.22-3.11 (m, 1H), 3.06 (d, J=6.8 Hz, 1H), 2.78 (d, J=2.7 Hz, 3H), 2.68-2.47 (m, 1H), 2.46-1.95 (m, 5H), 1.89-1.26 (m, 1H).

Example 84. Compounds 240-243 were prepared in an analogous fashion as that described for Compound 239.

| Compound | | | |
|---|---|---|---|
| Number | Structure | Chemical Name | MS: (M + H)⁺ & ¹H MNR |
| 240 | 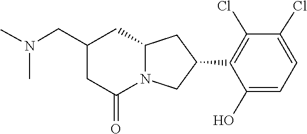 | (2R,8aR)-2-(2,3- dichloro-6- hydroxyphenyl)- 7- [(dimethylamino) methyl]- hexahydro-1H- indolizin-5-one | [M + H]⁺: 357, 359 (3:2); ¹H NMR (400 MHz, CD₃OD) δ 7.26 (d, J = 8.8 Hz, 1H), 6.76 (d, J = 8.7 Hz, 1H), 4.34-4.23 (m, 1H), 4.16 (dd, J = 11.5, 8.9 Hz, 1H), 3.88-3.80 (m, 1H), 3.57-3.51 (m, 1H), 3.33-3.16 (m, 2H), 2.97 (d, J = 2.7 Hz, 6H), 2.70-2.60 (m, |
| | | | 2H), 2.46-2.11 (m, 4H), 1.88-1.79 |
| | | | (m, 1H). |
| 241 | 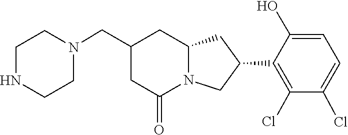 [SMILES: O=C1CC(CN2CCNCC2)C[C@@H]2C[C@H](c3c(O)ccc(Cl)c3Cl)CN12] | (2R,8aR)-2-(2,3- dichloro-6- hydroxyphenyl)- 7-(piperazin-1- ylmethyl)- hexahydro-1H- indolizin-5-one | [M + H]⁺: 398, 400 (3:2); ¹H NMR (400 MHz, D₂O) δ 7.22 (dd, J = 8.8, 1.7 Hz, 1H), 6.72 (dd, J = 8.8, 1.7 Hz, 1H), 4.23-4.14 (m, 1H), 4.03-3.95 (m, 1H), 3.79-3.69 (m, 1H), 3.65-3.53 (m, 8H), 3.52- 3.43 (m, 1H), 3.40-3.24 (m, 2H), 2.71-2.50 (m, 2H), 2.30-2.05 (m, |
| | | | 4H), 1.85-1.75 (m, 1H). |
| 242 | 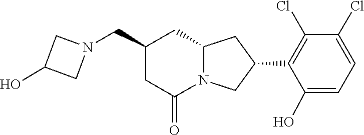 [SMILES: O=C1C[C@@H](CN2CC(O)C2)C[C@@H]2C[C@H](c3c(O)ccc(Cl)c3Cl)CN12] | (2R,8aR)-2-(2,3- dichloro-6- hydroxyphenyl)- 7-[(3- hydroxyazetidin- 1-yl)methyl]- hexahydro-1H- indolizin-5-one | [M + H]⁺: 385, 387 (3:2); ¹H NMR (400 MHz, CD₃OD) δ 7.24 (d, J = 8.7 Hz, 1H), 6.75 (d, J = 8.8 Hz, 1H), 4.41-4.34 (m, 1H), 4.30-4.21 (m, 1H), 4.11 (dd, J = 11.6, 9.2 Hz, 1H), 3.80-3.71 (m, 3H), 3.56-3.46 (m, 1H), 3.02-2.95 (m, 2H), 2.60-2.46 (m, 3H), 2.41- |
| | | isomer 2 | 2.32 (m, 1H), 2.26-2.11 (m, 2H), |
| | | | 2.07-1.97 (m, 2H), 1.24-1.14 (m, |
| | | | 1H). |
| 243 | 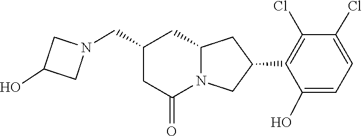 [SMILES: O=C1C[C@H](CN2CC(O)C2)C[C@@H]2C[C@H](c3c(O)ccc(Cl)c3Cl)CN12] | (2R,8aR)-2-(2,3- dichloro-6- hydroxyphenyl)- 7-[(3- hydroxyazetidin- 1-yl)methyl]- hexahydro-1H- indolizin-5-one isomer 2 | [M + H]⁺: 385, 387 (3:2); ¹H NMR (400 MHz, CD₃OD) δ 7.24 (d, J = 8.8 Hz, 1H), 6.74 (d, J = 8.8 Hz, 1H), 4.43-4.37 (m, 1H), 4.30-4.20 (m, 1H), 4.13 (dd, J = 11.5, 8.8 Hz, 1H), 3.85-3.72 (m, 3H), 3.54-3.45 (m, 1H), 3.05-2.96 (m, 2H), 2.68 (dd, J = 11.9, 7.6 Hz, 1H), 2.57 (dd, J = 11.9, 7.2 |
| | | | Hz, 1H), 2.50 (dd, J = 17.7, 6.2 |
| | | | Hz, 1H), 2.40-2.31 (m, 1H), 2.31- |
| | | | 2.19 (m, 1H), 2.22-2.07 (m, 3H), |
| | | | 1.70-1.62 (m, 1H). |

### Example 85. Compound 244 ((6R,7aR)-6-(2,3-dichloro-6-hydroxyphenyl)-hexahydropyrrolizin-3-one)

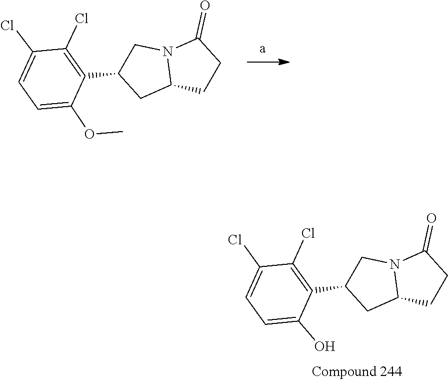 [SMILES: COc1ccc(Cl)c(Cl)c1[C@H]1C[C@H]2CCC(=O)N2C1 ; O=C1CC[C@@H]2C[C@H](c3c(O)ccc(Cl)c3Cl)CN12]

To a solution of (6R,7aR)-6-(2,3-dichloro-6-methoxyphenyl)-hexahydropyrrolizin-3-one (Intermediate 18, Example 16) (50.0 mg, 0.17 mmol) in DCM (2 mL) was added BBr₃ (0.10 mL, 1.06 mmol) at room temperature. The reaction was then stirred at room temperature for 1 h, quenched with MeOH (2 mL) at 0° C. and concentrated under reduced pressure. The residue was purified with Prep-HPLC with the following conditions: Column: X Select CSH Prep C18 OBD Column, 19×250 mm, 5 μm; Mobile Phase A: Water (plus 0.1% FA), Mobile Phase B: ACN; Flow rate: 20 mL/min; Gradient: 40% B to 90% B in 5.5 min; Detector: UV 254/210 nm; Retention time: 5.50 min. The fractions containing the desired product were collected and concentrated under reduced pressure to afford Compound 244 ((6R,7aR)-6-(2,3-dichloro-6-hydroxyphenyl)-hexahydropyrrolizin-3-one) as an off-white solid (12.8 mg, 27%): LCMS (ESI) calc'd for C₁₃H₁₃Cl₂NO₂ [M+H]⁺: 286, 288 (3:2) found 286, 288 (3:2); ¹H NMR (400 MHz, CD₃OD) δ 7.24 (d, J=8.8 Hz, 1H), 6.75 (d, J=8.7 Hz, 1H), 4.56-4.41 (m, 1H), 4.25-4.09 (m, 1H), 3.94 (dd, J=11.1, 7.7 Hz, 1H), 3.31-3.21 (m, 1H), 2.87-2.71 (m, 1H), 2.58-2.47 (m, 1H), 2.45-2.34 (m, 1H), 2.20-2.02 (m, 2H), 1.98-1.85 (m, 1H).

### Example 86. Compound 245 ((6R₇aS)-6-(2,3-dichloro-6-hydroxyphenyl)-2-hydroxy-hexahydropyrrolizin-3-one isomer 1) and Compound 246 ((6R,7aS)-6-(2,3-dichloro-6-hydroxyphenyl)-2-hydroxy-hexahydropyrrolizin-3-one isomer 2)

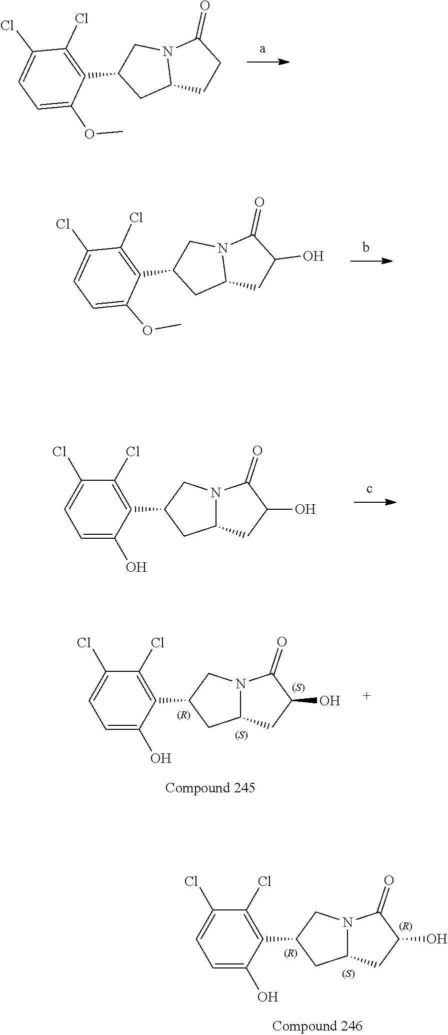

Step a:

To a solution of i-Pr₂NH (0.100 g, 1.00 mmol) in THE (2 mL) was added n-BuLi (0.28 mL, 0.70 mmol, 2.5 M in hexane) dropwise at −65° C. under nitrogen atmosphere over 5 min. After 15 min, a solution of (6R,7aR)-6-(2,3-dichloro-6-methoxyphenyl)-hexahydropyrrolizin-3-one (Intermediate 18, Example 16) (0.200 g, 0.67 mmol) in THE (2 mL) was added dropwise at −65° C. After 40 min, a solution of 2-(benzenesulfonyl)-3-phenyloxaziridine (0.260 g, 1.00 mmol) in THE (2 mL) was added dropwise at −65° C. The reaction was stirred at −65° C. for 1 h allowed to warm up to room temperature over 16 h and quenched with saturated aq. NH₄Cl (10 mL). The resulting mixture was extracted with EA (2×20 mL). The combined organic phases were washed with brine (3×20 mL) and dried over anhydrous Na₂SO₄. After filtration, the filtrate was concentrated under reduced pressure. The residue was purified by reverse phase chromatography, eluting with 65% ACN in water (plus 0.05% TFA) to afford (6R,7aS)-6-(2,3-dichloro-6-methoxyphenyl)-2-hydroxy-hexahydropyrrolizin-3-one as a colorless oil (60.0 mg, 29%): LCMS (ESI) calc'd for C₁₄H₁₅Cl₂NO₃ [M+H]⁺: 316, 318 (3:2) found 316, 318 (3:2).

Step b:

To a solution of (6R,7aS)-6-(2,3-dichloro-6-methoxyphenyl)-2-hydroxy-hexahydropyrrolizin-3-one (60.0 mg, 0.19 mmol) in DCM (2 mL) was added BBr₃ (0.25 mL, 2.64 mmol) at room temperature. The reaction was stirred at room temperature for 1 h, quenched with MeOH (5 mL) at 0° C. and concentrated under reduced pressure. The residue was purified by Prep-HPLC with the following conditions: Column: X Select CSH Prep C18 OBD Column, 19 mm×250 mm, 5 μm; Mobile Phase A: water (plus 0.05% TFA), Mobile Phase B: ACN; Flow rate: 20 mL/min; Gradient: 35% B to 65% B in 5.5 min; Detector: UV 210 nm; Retention time: 5.6 min. The fractions containing the desired product were collected and concentrated under reduced pressure to afford (6R,7aS)-6-(2,3-dichloro-6-hydroxyphenyl)-2-hydroxy-hexahydropyrrolizin-3-one as an off-white solid (34.0 mg, 59%): LCMS (ESI) calc'd for C₁₃H₁₃Cl₂NO₃ [M+H]⁺: 302, 304 (3:2) found 302, 304 (3:2); ¹H NMR (400 MHz, CD₃OD) δ 7.23 (dd, J=8.8 Hz, 1H), 6.74 (d, J=9.0 Hz, 1H), 4.71-4.34 (m, 2H), 4.25-3.87 (m, 2H), 3.38-3.35 (m, 0.5H), 3.32-3.23 (m, 0.5H), 2.86-2.74 (m, 0.5H), 2.28-2.08 (m, 2.5H), 2.04-1.64 (m, 1H).

Step c:

6R,7aS)-6-(2,3-dichloro-6-hydroxyphenyl)-2-hydroxy-hexahydropyrrolizin-3-one (31.7 mg, 0.11 mmol) was separated by Prep Chiral HPLC with the following condition: Column: CHIRALPAK IE, 2×25 cm, 5 μm; Mobile Phase A: Hex (plus 0.1% FA), Mobile Phase B: IPA; Flow rate: 15 mL/min; Gradient: 30% B to 30% B in 9 min; Detector: UV 220/254 nm; Retention time 1: 5.41 min; Retention time 2: 6.74 min; The faster-eluting isomer at 5.41 min was obtained Compound 245 ((6R,7aS)-6-(2,3-dichloro-6-hydroxyphenyl)-2-hydroxy-hexahydropyrrolizin-3-one isomer 1) as an off-white solid (9.30 mg, 29%): LCMS (ESI) calc'd for C₁₃H₁₃Cl₂NO₃ [M+H]⁺: 302, 304 (3:2) found 302, 304 (3:2); ¹H NMR (300 MHz, CD₃OD) δ 7.24 (d, J=8.7 Hz, 1H), 6.74 (d, J=8.8 Hz, 1H), 4.60-4.35 (m, 2H), 4.31-4.12 (m, 1H), 3.92 (dd, J=11.4, 7.4 Hz, 1H), 3.42-3.35 (m, 1H), 2.30-2.09 (m, 3H), 2.00-1.95 (m, 1H). The slower-eluting isomer at 6.74 min was obtained Compound 246 ((6R,7aS)-6-(2,3-dichloro-6-hydroxyphenyl)-2-hydroxy-hexahydropyrrolizin-3-one isomer 2) as an off-white solid (9.40 mg, 30%): LCMS (ESI) calc'd for C₁₃H₁₃Cl₂NO₃ [M+H]⁺: 302, 304 (3:2) found 302, 304 (3:2); ¹H NMR (300 MHz, CD₃OD) δ 7.25 (d, J=8.8 Hz, 1H), 6.76 (d, J=8.8 Hz, 1H), 4.74-4.59 (m, 1H), 4.58-4.40 (m, 1H), 4.09-3.84 (m, 2H), 3.31-3.20 (m, 1H), 2.87-2.73 (m, 1H), 2.28-2.03 (m, 2H), 1.83-1.67 (m, 1H).

### Example 87. Compound 247 ((6R,7aS)-6-(2,3-dichloro-6-hydroxyphenyl)-2-(hydroxymethyl)-hexahydropyrrolizin-3-one)

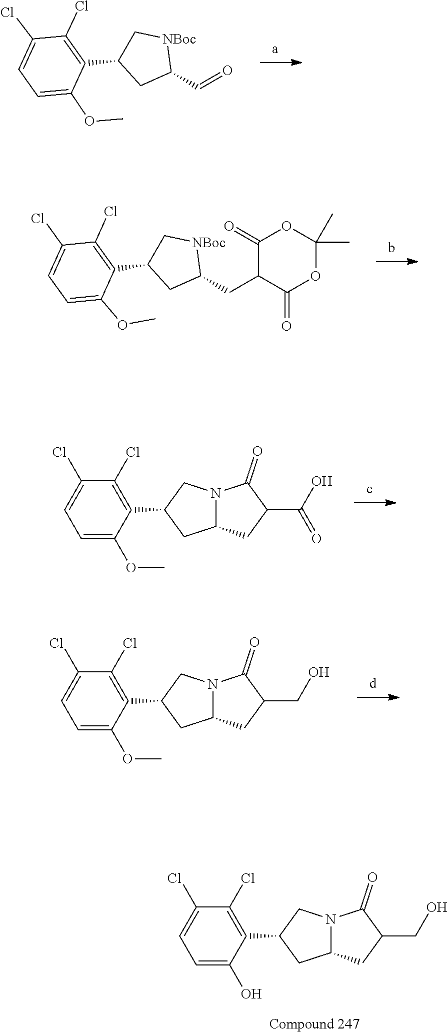

Step a:

To a stirred solution of tert-butyl (2S,4R)-4-(2,3-dichloro-6-methoxyphenyl)-2-formylpyrrolidine-1- carboxylate (1.00 g, 2.67 mmol), Meldrum's acid (0.390 g, 2.67 mmol) and etidin (0.680 g, 2.67 mmol) in MeCN (10 mL) was added L-proline (31.0 mg, 0.27 mmol) at room temperature. The reaction was stirred for 4 h at room temperature, concentrated under reduced pressure, diluted with MeOH (10 mL) and filtered. The filter cake was washed with MeOH (2×10 mL). The filtrate was concentrated under reduced pressure. The residue was purified by reverse phase chromatography, eluting with 60% ACN in water (plus 0.05% TFA) to afford tert-butyl (2S,4R)-4-(2,3-dichloro-6-methoxyphenyl)-2-[(2,2-dimethyl-4,6-dioxo-1,3-dioxan-5-yl)methyl]pyrrolidine-1-carboxylate as a light yellow oil (1.20 g, 89%): LCMS (ESI) calc'd for C₂₃H₂₉Cl₂NO₇ [M+H]⁺: 502, 504 (3:2) found 502, 504 (3:2); ¹H NMR (400 MHz, CDCl₃) δ 7.35 (d, J=8.9 Hz, 1H), 6.79 (d, J=8.9 Hz, 1H), 4.91-4.81 (m, 1H), 4.57-4.41 (m, 1H), 4.10-3.94 (m, 1H), 3.91 (s, 3H), 3.86-3.73 (m, 2H), 2.68-2.40 (m, 2H), 2.28-2.13 (m, 2H), 1.84 (d, J=35.4 Hz, 6H), 1.46 (s, 9H).

Step b:

A solution of tert-butyl (2S,4R)-4-(2,3-dichloro-6-methoxyphenyl)-2-[(2,2-dimethyl-4,6-dioxo-1,3-dioxan-5-yl)methyl]pyrrolidine-1-carboxylate (0.770 g, 1.53 mmol) and TFA (1 mL) in DCM (4 mL) was stirred for 1 h at room temperature and concentrated under reduced pressure. The residue was dissolved in EtOH (5 mL) and basified to pH 8 with TEA. The resulting mixture was stirred at 80° C. for 1 h and concentrated under reduced pressure. The residue was purified by reverse phase chromatography, eluting with 40% ACN in water (plus 0.05% TFA) to afford (6R,7aS)-6-(2,3-dichloro-6-methoxyphenyl)-3-oxo-hexahydropyrrolizine-2-carboxylic acid as a light yellow oil (0.440 g, 83%): LCMS (ESI) calc'd for C₁₅H₁₅Cl₂NO₄ [M+H]⁺: 344, 346 (3:2) found 344, 346 (3:2); ¹H NMR (300 MHz, CDCl₃) δ 7.39-7.29 (m, 1H), 6.77 (d, J=8.8 Hz, 1H), 4.52 (dd, J=17.6, 10.1 Hz, 1H), 4.18-4.01 (m, 1H), 4.00-3.85 (m, 1H), 3.85-3.61 (m, 4H), 3.40-3.22 (m, 1H), 2.90-2.66 (m, 1H), 2.43-2.10 (m, 2H), 2.01-1.72 (m, 1H).

Step c:

A solution of (6R,7aS)-6-(2,3-dichloro-6-methoxyphenyl)-3-oxo-hexahydropyrrolizine-2-carboxylic acid (0.440 g, 1.28 mmol), 2-methylpropyl chloroformate (0.350 g, 2.56 mmol) and 4-methylmorpholine (0.260 g, 2.56 mmol) in DME (5 mL) was stirred at 0° C. for 1 h under nitrogen atmosphere. To the above mixture was added NaBH₄ (97.0 mg, 2.56 mmol) at room temperature. The resulting mixture was stirred for additional 16 h and quenched with saturated aq. NH₄Cl (20 mL). The resulting mixture was extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 0.05% TFA) to afford (6R,7aS)-6-(2,3-dichloro-6-methoxyphenyl)-2-(hydroxymethyl)-hexahydropyrrolizin-3-one as a light yellow oil (93 mg, 22%): LCMS (ESI) calc'd for C₁₅H₁₇Cl₂NO₃ [M+H]⁺: 330, 332 (3:2) found 330, 332 (3:2).

Step d:

A solution of (6R,7aS)-6-(2,3-dichloro-6-methoxyphenyl)-2-(hydroxymethyl)-hexahydropyrrolizin-3-one (90.0 mg, 0.27 mmol) and BBr₃ (0.13 mL, 1.38 mmol) in DCM (3 mL) was stirred for 20 min at room temperature, quenched with MeOH (2 mL) at 0° C. and concentrated under reduced pressure. The residue was purified by Prep-HPLC with the following conditions: Column: X Bridge Shield RP18 OBD Column, 19×250 mm, 10 μm; Mobile Phase A: Water (plus 10 mM NH₄HCO₃), Mobile Phase B: ACN; Flow rate: 20 mL/min; Gradient: 45% B to 50% B in 5.5 min; Detector: UV 210 nm; Retention time: 5.55 min. The fractions containing the desired product were collected and concentrated under reduced pressure to afford Compound 247 ((6R,7aS)-6-(2,3-dichloro-6-hydroxyphenyl)-2-(hydroxymethyl)-hexahydropyrrolizin-3-one) as an off-white solid (43.5 mg, 50%): LCMS (ESI) calc'd for C₁₄H₁₅Cl₂NO₃ [M+H]⁺: 316, 318 (3:2) found 316, 318 (3:2); ¹H NMR (400 MHz, CD₃OD) δ 7.23 (d, J=8.9 Hz, 1H), 6.74 (d, J=8.7 Hz, 1H), 4.54-4.37 (m, 1H), 4.18-4.04 (m, 1H), 3.99-3.91 (m, 1H), 3.86 (td, J=10.2, 9.7, 4.9 Hz, 1H), 3.79-3.69 (m, 1H), 3.31-3.23 (m, 1H), 3.11-2.78 (m, 1H), 2.60-2.31 (m, 1H), 2.22-1.77 (m, 3H).

### Example 88. Compound 248 ((6R,7aS)-2-amino-6-(2,3-dichloro-6-hydroxyphenyl)-hexahydropyrrolizin-3-one isomer 1) and Compound 249 ((6R,7aS)-2-amino-6-(2,3-dichloro-6-hydroxyphenyl)-hexahydropyrrolizin-3-one isomer 2)

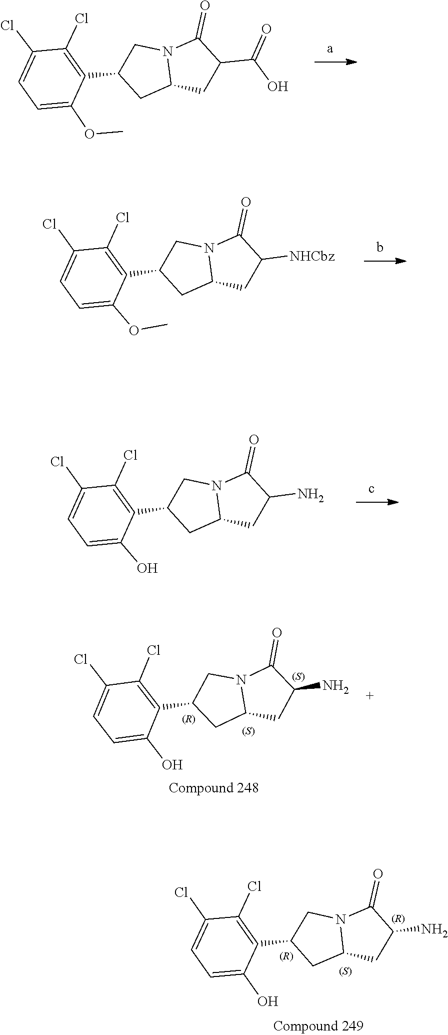

Step a:

To a stirred solution of (6R,7aS)-6-(2,3-dichloro-6-methoxyphenyl)-3-oxo-hexahydropyrrolizine-2- carboxylic acid (0.400 g, 1.16 mmol) in toluene (4 mL) were added TEA (0.65 mL, 6.39 mmol) and DPPA (1 mL, 3.65 mmol) at room temperature under nitrogen atmosphere. The reaction was stirred at 100° C. for 2 h. To the reaction mixture benzyl alcohol (4 mL) was added dropwise at room temperature. The resulting mixture was stirred at 100° C. for additional 2 h. The resulting mixture was diluted with water (20 mL) followed by extraction with DCM (3×20 mL). The combined organic layers were washed with brine (3×20 mL) and dried over anhydrous Na₂SO₄. After filtration, the filtrate was concentrated under reduced pressure. The residue was purified by silica gel column chromatography, eluting with EA/PE (1/1) and DCM to afford benzyl N-[(6R,7aS)-6-(2,3-dichloro-6-methoxyphenyl)-3-oxo-hexahydropyrrolizin-2-yl]carbamate as an off-white solid (60.0 mg, 11%): LCMS (ESI) calc'd for C₂₂H₂₂Cl₂N₂O₄ [M+H]⁺: 449, 451 (3:2) found 449, 451 (3:2). ¹H NMR (400 MHz, CDCl₃) δ 7.46-7.31 (m, 6H), 6.77 (d, J=8.9 Hz, 1H), 5.53 (s, 1H), 5.16 (s, 2H), 4.54 (d, J=46.9 Hz, 2H), 4.08-3.87 (m, 2H), 3.81 (s, 3H), 3.41-3.23 (m, 1H), 3.13-2.98 (m, 1H), 2.32-2.14 (m, 1H), 2.01-1.84 (m, 1H), 1.84-1.69 (m, 1H).

Step b:

A solution of benzyl N-[(6R,7aS)-6-(2,3-dichloro-6-methoxyphenyl)-3-oxo-hexahydropyrrolizin-2-yl] carbamate (50.0 mg, 0.11 mmol) and BBr₃ (0.05 mL, 0.53 mmol) in DCM (1 mL) was stirred at room temperature for 1 h, quenched with MeOH (2 mL) at 0° C. and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 20% B to 50% B in 5.5 min; Detector: UV 210 nm; Retention time: 5.56 min. The fractions containing the desired product were collected and concentrated under reduced pressure to afford (6R,7aS)-2-amino-6-(2,3-dichloro-6- hydroxyphenyl)-hexahydropyrrolizin-3-one as an off-white solid (16.0 mg, 34%): LCMS (ESI) calc'd for C₁₃H₁₄Cl₂N₂O₂ [M+H]⁺: 301, 303 (3:2) found 301, 303 (3:2); ¹H NMR (400 MHz, CD₃OD) δ 7.25 (d, J=8.8 Hz, 1H), 6.78 (d, J=8.8 Hz, 1H), 4.63-4.45 (m, 1H), 4.42 (dd, J=11.6, 7.8 Hz, 1H), 4.23-4.10 (m, 1H), 4.10-4.05 (m, 1H), 3.45-3.35 (m, 1H), 2.94-2.83 (m, 1H), 2.55-2.15 (m, 2H), 2.03-1.87 (m, 1H).

Step c:

(6R,7aS)-2-amino-6-(2,3-dichloro-6-hydroxyphenyl)-hexahydropyrrolizin-3-one (14.0 mg, 0.03 mmol) was separated by Prep Chiral HPLC with the following conditions: Column: CHIRALPAK IE, 2×25 cm, 5 μm; Mobile Phase A: Hex/DCM=3/1 (plus 10 mM NH₃-MeOH)-HPLC, Mobile Phase B: EtOH-HPLC; Flow rate: 20 mL/min; Gradient: 20% B to 20% B in 9 min; Detector: UV 220/254 nm; Retention Time 1: 5.13 min; Retention Time 2: 6.76 min; Injection Volume: 1 mL; Number Of Runs: 2. The faster-eluting isomer at 5.13 min was obtained Compound 248 ((6R,7aS)-2-amino-6-(2,3-dichloro-6-hydroxyphenyl)-hexahydropyrrolizin-3-one isomer 1) as an off-white solid (0.7 mg, 7%): LCMS (ESI) calc'd for C₁₃H₁₄Cl₂N₂O₂ [M+H]⁺: 301, 303 (3:2) found 301, 303 (3:2); ¹H NMR (400 MHz, CD₃OD) δ 7.23 (d, J=8.8 Hz, 1H), 6.73 (d, J=8.7 Hz, 1H), 4.56-4.40 (m, 1H), 4.22-4.11 (m, 1H), 3.92 (dd, J=11.3, 7.0 Hz, 1H), 3.71 (dd, J=8.9, 4.4 Hz, 1H), 3.37-3.33 (m, 1H), 2.32-2.22 (m, 1H), 2.18-2.07 (m, 2H), 2.02-1.96 (m, 1H). The slower-eluting isomer at 6.76 min was obtained Compound 249 ((6R,7aS)-2-amino-6-(2,3-dichloro-6-hydroxyphenyl)-hexahydropyrrolizin-3-one isomer 2) as an off-white solid (3.3 mg, 32.50%): LCMS (ESI) calc'd for C₁₃H₁₄Cl₂N₂O₂ [M+H]⁺: 301, 303 (3:2) found 301, 303 (3:2); ¹H NMR (400 MHz, CD₃OD) δ 7.25 (d, J=8.8 Hz, 1H), 6.76 (d, J=8.8 Hz, 1H), 4.57-4.43 (m, 1H), 4.06-3.86 (m, 3H), 3.30-3.23 (m, 1H), 2.82-2.67 (m, 1H), 2.23-2.04 (m, 2H), 1.69-1.60 (m, 1H).

### Example 89. Compound 250 ((8R,9aS)-8-(2,3-dichloro-6-hydroxyphenyl)-2-hydroxy-octahydroquinolizin-4-one)

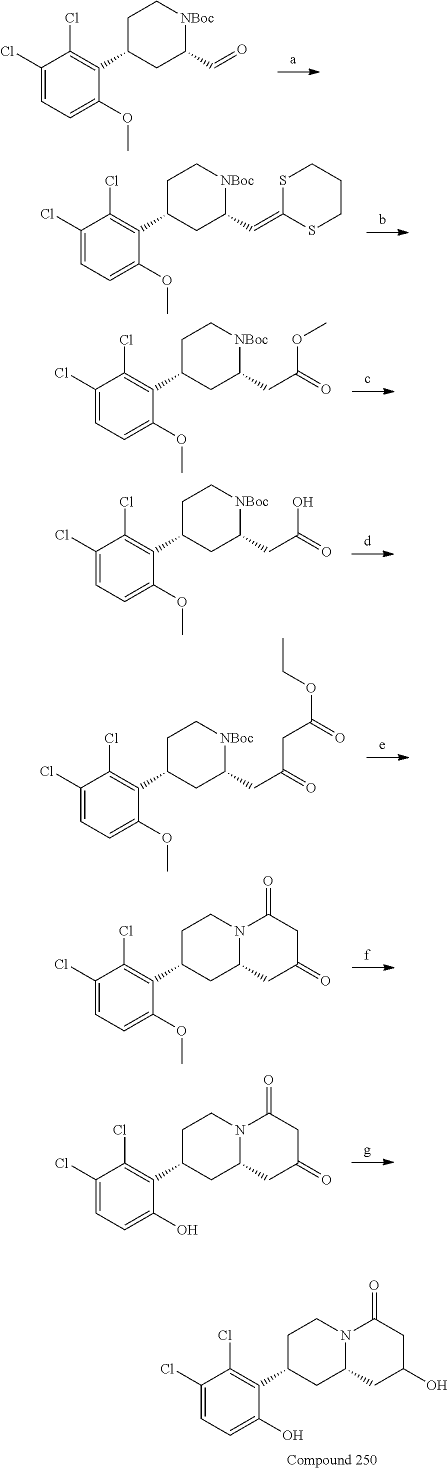

Step a:

To a stirred solution of diethyl 1,3-dithian-2-ylphosphonate (1.98 g, 7.73 mmol) in THE (30 mL) was added n-BuLi (3.14 mL, 7.854 mmol, 2.5 Min Hexane) dropwise at −78° C. under nitrogen atmosphere. The reaction was stirred at −78° C. for 1 h. Then tert-butyl (2S,4R)-4-(2,3-dichloro-6-methoxyphenyl)-2-formylpiperidine-1-carboxylate (Intermediate 10, Example 8) (2.50 g, 6.44 mmol) was added. The reaction was stirred at −78° C. to −30° C. for an additional 1 h, quenched with water (30 mL) and extracted with EA (3×30 mL). The combined organic layers were washed with brine (3×30 mL) and dried over anhydrous Na₂SO₄. After filtration, the filtrate was concentrated under reduced pressure. The residue was purified by silica gel column chromatography, eluting with PE/EA (3/1) to afford tert-butyl (2S,4R)-4-(2,3-dichloro-6-methoxyphenyl)-2-(1,3-dithian-2-ylidenemethyl)piperidine-1-carboxylate as a yellow oil (2.20 g, 63%): LCMS (ESI) calc'd for C₂₂H₂₉Cl₂NO₃S2 [M+H]⁺: 490, 492 (3:2) found 490, 492 (3:2); ¹H NMR (400 MHz, CDCl₃) δ 6.74 (d, J=8.9 Hz, 1H), 6.01 (d, J=7.9 Hz, 1H), 4.73-4.62 (m, 1H), 3.87-3.76 (m, 5H), 3.74-3.60 (m, 1H), 3.49-3.37 (m, 1H), 3.02-2.85 (m, 3H), 2.88-2.75 (m, 1H), 2.39-2.24 (m, 1H), 2.23-2.12 (m, 2H), 1.99-1.87 (m, 2H), 1.68-1.62 (m, 1H), 1.52 (s, 9H).

Step b:

To a stirred solution of tert-butyl (2S,4R)-4-(2,3-dichloro-6-methoxyphenyl)-2-(1,3-dithian-2-ylidenemethyl)piperidine-1-carboxylate (1.20 g, 2.45 mmol) in MeOH (10 mL) was added CuSO₄.5H₂O (3.05 g, 12.23 mmol) at room temperature. The reaction was stirred at 65° C. for 1 h. The resulting mixture was filtered and the filter cake washed with MeOH (2×5 mL). The filtrate was concentrated under reduced pressure. The residue was purified by silica gel column chromatography, eluting with PE/EA (3/1) to afford tert-butyl (2S,4R)-4-(2,3-dichloro-6-methoxyphenyl)-2-(2-methoxy-2-oxoethyl)piperidine-1-carboxylate as a light yellow oil (0.540 g, 46%): LCMS (ESI) calc'd for C₂₀H₂₇Cl₂NO₅ [M+H]⁺: 432, 434 (3:2) found 432, 434 (3:2); ¹H NMR (400 MHz, CD₃OD) δ 7.38 (d, J=8.9 Hz, 1H), 6.97 (d, J=9.0 Hz, 1H), 4.20-4.06 (m, 1H), 3.87 (s, 3H), 3.81-3.74 (m, 1H), 3.70-3.52 (m, 4H), 3.47-3.36 (m, 1H), 2.80-2.56 (m, 2H), 2.51-2.37 (m, 1H), 1.99-1.85 (m, 2H), 1.73-1.63 (m, 1H), 1.51 (s, 9H).

Step c:

To a stirred solution of tert-butyl (2S,4R)-4-(2,3-dichloro-6-methoxyphenyl)-2-(2-methoxy-2-oxoethyl)piperidine-1-carboxylate (0.540 g, 1.25 mmol) in MeOH (4 mL) and H₂O (2 mL) was added LiOH H₂O (0.100 g, 2.50 mmol) at room temperature. The reaction was stirred for 1 h at 40° C. The resulting mixture was acidified to pH 5 with citric acid followed by extraction with EA (3×20 mL). The combined organic layers were washed with brine (2×20 mL) and dried over anhydrous Na₂SO₄. After filtration, the filtrate was concentrated under reduced pressure to afford [(2S,4R)-1-(tert-butoxycarbonyl)-4-(2,3-dichloro-6-methoxyphenyl)pyrrolidin-2-yl]acetic acid as a yellow oil (0.500 g, crude), which was used in the next step directly without purification: LCMS (ESI) calc'd for C₁₉H₂₅Cl₂NO₅ [M+H]⁺: 418, 420 (3:2) found 418, 420 (3:2).

Step d:

To a stirred solution of [(2S,4R)-1-(tert-butoxycarbonyl)-4-(2,3-dichloro-6-methoxyphenyl)pyrrolidin-2-yl]acetic acid (0.500 g, 1.20 mmol) and Meldrum's acid (0.260 g, 1.79 mmol) in DCM (5 mL) were added DMAP (0.220 g, 1.79 mmol) and EDCI (0.340 g, 1.79 mmol) at room temperature. The reaction was stirred for 1 h. The resulting mixture was washed with aq. HCl (1 M, 2×20 mL). The organic layer was washed with brine (2×20 mL) and dried over anhydrous Na₂SO₄. After filtration, the filtrate was concentrated under reduced pressure. The residue was dissolved in EtOH (5 mL), stirred at 90° C. for 1 h and concentrated under reduced pressure. The residue was purified by reverse phase chromatography, eluting with 75% ACN in water (plus 0.05% TFA) to afford tert-butyl (2S,4R)-4-(2,3-dichloro-6-methoxyphenyl)-2-(4-ethoxy-2,4-dioxobutyl)piperidine-1-carboxylate as a light yellow oil (0.360 g, 59% overall two steps): LCMS (ESI) calc'd for C₂₃H₃₁Cl₂NO₆ [M+H]⁺: 488, 490 (3:2) found 488, 490 (3:2); ¹H NMR (400 MHz, CDCl₃) δ 7.31 (d, J=8.9 Hz, 1H), 6.74 (d, J=8.9 Hz, 1H), 4.28-4.04 (m, 3H), 3.83 (s, 3H), 3.71-3.63 (m, 2H), 3.53-3.41 (m, 3H), 3.11 (dd, J=16.3, 5.1 Hz, 1H), 2.82 (dd, J=16.2, 7.7 Hz, 1H), 2.42-2.23 (m, 1H), 2.02-1.79 (m, 3H), 1.52 (s, 9H), 1.35-1.24 (m, 3H).

Step e:

To a stirred solution of tert-butyl (2S,4R)-4-(2,3-dichloro-6-methoxyphenyl)-2-(4-ethoxy-2,4-dioxobutyl)piperidine-1-carboxylate (0.340 g, 0.70 mmol) in DCM (4 mL) was added TFA (1 mL) dropwise at room temperature. The reaction was stirred for 1 h and concentrated under reduced pressure. Then to the residue in MeOH (3.5 mL) was added K₂CO₃ (0.480 g, 3.48 mmol) at room temperature and stirred for an additional 1 h. The resulting mixture was diluted with water (20 mL) followed by extraction with EA (3×20 mL). The combined organic layers were washed with brine (2×20 mL) and dried over anhydrous Na₂SO₄. After filtration, the filtrate was concentrated under reduced pressure The residue was purified by reverse phase chromatography, eluting with 45% ACN in water (plus 0.05% TFA) to afford (8R,9aS)-8-(2,3-dichloro-6-methoxyphenyl)-hexahydro-1H-quinolizine-2,4-dione as a light yellow oil (87.0 mg, 33%): LCMS (ESI) calc'd for C₁₆H₁₇Cl₂NO₃ [M+H]⁺: 342, 344 (3:2) found 342, 344 (3:2); ¹H NMR (400 MHz, CD₃OD) δ 7.39 (d, J=9.0 Hz, 1H), 6.96 (d, J=9.0 Hz, 1H), 4.74 (d, J=9.9 Hz, 1H), 3.84 (d, J=5.7 Hz, 3H), 3.76-3.62 (m, 2H), 3.48-3.34 (m, 2H), 2.72-2.58 (m, 1H), 2.44-2.20 (m, 3H), 1.84-1.52 (m, 3H).

Step f:

To a stirred solution of (8R,9aS)-8-(2,3-dichloro-6-methoxyphenyl)-hexahydro-1H-quinolizine-2,4-dione (86.0 mg, 0.25 mmol) in DCM (1 mL) was added BBr₃ (0.24 mL, 0.95 mmol) at room temperature. The reaction was stirred for 1 h, quenched with water (5 mL) at 0° C. and extracted with EA (3×20 mL). The combined organic layers were washed with brine (2×20 mL) and dried over anhydrous Na₂SO₄. After filtration, the filtrate was concentrated under reduced pressure to afford (8R,9aS)-8-(2,3-dichloro-6-hydroxyphenyl)-hexahydro-1H-quinolizine-2,4-dione as a yellow oil (60.0 mg, crude), which was used in the next step directly without purification: LCMS (ESI) calc'd for C₁₅H₁₅Cl₂NO₃ [M+H]⁺: 328, 330 (3:2) found 328, 330 (3:2).

Step g:

To a stirred mixture of (8R,9aS)-8-(2,3-dichloro-6-hydroxyphenyl)-hexahydro-1H-quinolizine-2,4-dione (60.0 mg, 0.18 mmol) in THE (1 mL) was added NaBH₄ (14.0 mg, 0.37 mmol) at room temperature. The reaction was stirred at room temperature for 1 h. The resulting mixture was quenched with saturated aq. NH₄Cl (20 mL) at 0° C. followed by extraction with EA (3×20 mL). The combined organic layers were washed with brine (2×20 mL) and dried over anhydrous Na₂SO₄. After filtration, the filtrate was concentrated under reduced pressure. The residue was purified by Prep-HPLC with the following conditions: Column: X Select CSH Prep C18 OBD Column, 19×250 mm, 5 μm; Mobile Phase A: water (plus 0.1% FA), Mobile Phase B: ACN; Flow rate: 20 mL/min; Gradient: 35% B to 65% B in 5.5 min; Detector: UV 210 nm; Retention time: 5.57 min. The fractions containing the desired product were collected and concentrated under reduced pressure to afford Compound 250 ((8R,9aS)-8-(2,3-dichloro-6-hydroxyphenyl)-2-hydroxy-octahydroquinolizin-4-one) as an off-white solid (19.0 mg, 31%): LCMS (ESI) calc'd for C₁₅H₁₇Cl₂NO₃ [M+H]⁺: 330, 332 (3:2) found 330, 332 (3:2); ¹H NMR (400 MHz, CD₃OD) δ 7.19 (d, J=8.8 Hz, 1H), 6.70 (d, J=8.8 Hz, 1H), 4.80-4.70 (m, 1H), 4.02-3.92 (m, 1H), 3.75-3.56 (m, 1H), 3.55-3.43 (m, 1H), 2.71-2.58 (m, 2H), 2.53-2.19 (m, 4H), 1.79-1.39 (m, 3H).

### Example 90. Compound 251 ((6R,7aS)-6-(2,3-dichloro-6-hydroxyphenyl)-tetrahydro-1H-pyrrolo[1,2-c][1,3]oxazol-3-one)

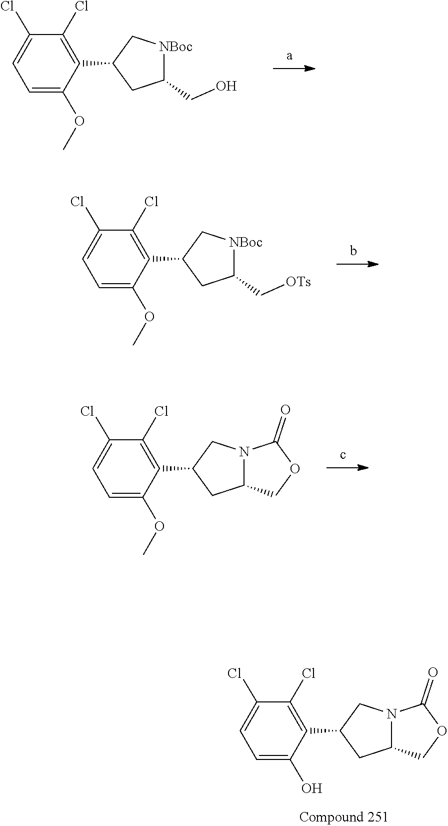 [SMILES: COc1ccc(Cl)c(Cl)c1[C@H]1C[C@@H](CO)N(C(=O)OC(C)(C)C)C1 ; COc1ccc(Cl)c(Cl)c1[C@H]1C[C@@H](COS(=O)(=O)c2ccc(C)cc2)N(C(=O)OC(C)(C)C)C1 ; COc1ccc(Cl)c(Cl)c1[C@H]1C[C@H]2COC(=O)N2C1 ; O=C1OC[C@@H]2C[C@H](c3c(O)ccc(Cl)c3Cl)CN12]

Step a:

To a stirred solution of tert-butyl (2S,4R)-4-(2,3-dichloro-6-methoxyphenyl)-2-(hydroxymethyl)pyrrolidine-1-carboxylate (Example 7, step b) (40.0 g, 95.68 mmol), TsCl (21.9 g, 115 mmol) and DMAP (3.51 g, 28.7 mmol) in DCM (400 mL) was added TEA (26.6 mL, 263 mmol) dropwise at room temperature. The reaction was stirred at room temperature for 4 h, diluted with water (100 mL) and extracted with DCM (2×200 mL). The combined organic layers were washed with brine (2×200 mL) and dried over anhydrous Na₂SO₄. After filtration, the filtrate was concentrated under reduced pressure. The residue was purified by silica gel column chromatography, eluting with PE/EA (3/1) to afford tert-butyl (2S,4R)-4-(2,3-dichloro-6-methoxyphenyl)-2-[[(4-methylbenzenesulfonyl)oxy]methyl]pyrrolidine-1-carboxylate as an off-white solid (42.5 g, 75%): LCMS (ESI) calc'd for C₂₄H₂₉Cl₂NO₆S [M+H]⁺: 530, 532 (3:2) found 530, 532 (3:2).

Step b:

To a stirred solution of tert-butyl (2S,4R)-4-(2,3-dichloro-6-methoxyphenyl)-2-[[(4-methylbenzenesulfonyl)oxy]methyl]pyrrolidine-1-carboxylate (12.0 g, 22.62 mmol) in DMSO (20 mL) was added KCN (2.95 g, 45.3 mmol) at room temperature. The reaction was stirred at 80° C. for 1 h, cooled to room temperature, diluted with saturated aq. NaHCO₃ (50 mL) and extracted with EA (3×50 mL). The combined organic layers were washed with brine (3×50 mL) and dried over anhydrous Na₂SO₄. After filtration, the filtrate was concentrated under reduced pressure. The residue was purified by silica gel chromatography, eluting with 35% EA in PE to afford (6R,7aS)-6-(2,3-dichloro-6-methoxyphenyl)tetrahydro-1H,3H-pyrrolo[1,2-c]oxazol-3-one as a yellow solid (2.50 g, 36%): LCMS (ESI) calc'd for C₁₃H₁₃Cl₂NO₃ [M+H]⁺: 302, 304 (3:2), found 302, 304 (3:2).

Step c:

To a stirred mixture of (6R,7aS)-6-(2,3-dichloro-6-methoxyphenyl)-tetrahydro-1H-pyrrolo[1,2-c][1,3]oxazol-3-one (50.0 mg, 0.16 mmol) in DCM (1 mL) was added BBr₃ (0.05 mL, 0.53 mmol) at room temperature. The reaction was stirred for 1 h, quenched with water (2 mL) and concentrated under reduced pressure. The residue was purified by Prep-HPLC with the following conditions: Column: X Bridge Shield RP18 OBD Column, 30×150 mm, 5 μm; Mobile Phase A: Water (plus 0.05% TFA), Mobile Phase B: ACN; Flow rate: 60 mL/min; Gradient: 25% B to 55% B in 7 min; Detector: UV 220/254 nm; Retention time: 6.8 min. The fractions containing the desired product were collected and concentrated under reduced pressure to afford Compound 251 ((6R,7aS)-6-(2,3-dichloro-6-hydroxyphenyl)-tetrahydro-1H-pyrrolo[1,2-c][1,3]oxazol-3-one) as an off-white solid (17.5 mg, 34.9%): LCMS (ESI) calc'd for C₁₂H₁₁Cl₂NO₃ [M+H]⁺: 288, 290 (3:2) found 288, 290 (3:2); ¹H NMR (400 MHz, CD₃OD) δ 7.24 (d, J=8.8 Hz, 1H), 6.74 (d, J=8.8 Hz, 1H), 4.58 (dd, J=9.0, 7.9 Hz, 1H), 4.46-4.34 (m, 1H), 4.32 (dd, J=9.0, 3.2 Hz, 1H), 4.25-4.14 (m, 1H), 3.93 (dd, J=10.7, 7.1 Hz, 1H), 3.46-3.40 (m, 1H), 2.29-2.09 (m, 2H).

### Example 91. Compound 252 ((7R,8aS)-7-(2,3-dichloro-6-hydroxyphenyl)-hexahydro-[1,3]oxazolo[3,4-a]pyridin-3-one)

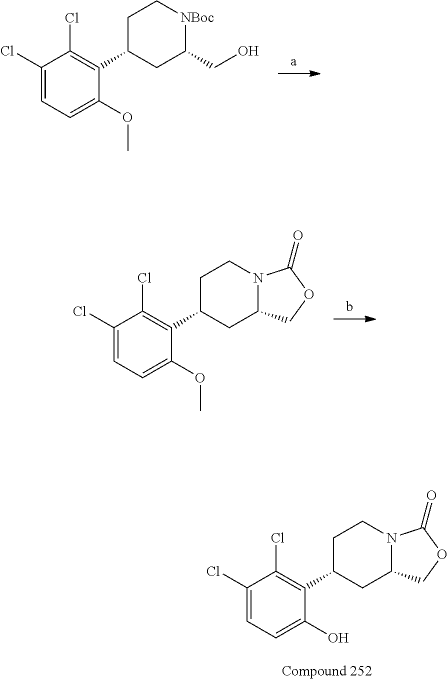 [SMILES: COc1ccc(Cl)c(Cl)c1[C@@H]1CCN(C(=O)OC(C)(C)C)[C@H](CO)C1 ; COc1ccc(Cl)c(Cl)c1[C@@H]1CCN2C(=O)OC[C@@H]2C1 ; O=C1OC[C@@H]2C[C@H](c3c(O)ccc(Cl)c3Cl)CCN12]

Step a:

To a stirred solution of tert-butyl (2S,4R)-4-(2,3-dichloro-6-methoxyphenyl)-2-(hydroxymethyl)piperidine-1-carboxylate (Intermediate 9, Example 8) (0.200 g, 0.51 mmol) and TsCl (0.150 g, 0.77 mmol) in DCM (2 mL) were added TEA (0.100 g, 1.03 mmol) and DMAP (19.0 mg, 0.15 mmol) at room temperature. The reaction was stirred at room temperature for 2 h, diluted with water (50 mL) and extracted with EA (3×20 mL). The combined organic layers were washed with brine (2×20 mL) and dried over anhydrous Na₂SO₄. After filtration, the filtrate was concentrated under reduced pressure. The residue was purified by silica gel column chromatography, eluting with PE/EA (3/1) to afford (7R,8aS)-7-(2,3-dichloro-6-methoxyphenyl)-hexahydro-[1,3]oxazolo[3,4-a]pyridin-3-one as an off-white semi-solid (0.120 g, 74%): LCMS (ESI) calc'd for C₁₄H₁₅Cl₂NO₃ [M+H]⁺: 316, 318 (3:2) found 316, 318 (3:2).

Step b:

To a stirred solution of (7R,8aS)-7-(2,3-dichloro-6-methoxyphenyl)-hexahydro-[1,3]oxazolo[3,4-a]pyridin-3-one (80.0 mg, 0.25 mmol) in DCM (1 mL) was added BBr₃ (0.640 g, 2.54 mmol) at room temperature. The reaction was stirred for 1 h, quenched with MeOH (3 mL) and concentrated under reduced pressure. The residue was purified by reverse phase chromatography, eluting with 35% ACN in water (plus 0.05% TFA) to afford Compound 252 ((7R,8aS)-7-(2,3-dichloro-6-hydroxyphenyl)-hexahydro-[1,3]oxazolo[3,4-a]pyridin-3-one) as an off-white solid (34.0 mg, 44%): LCMS (ESI) calc'd for C₁₃H₁₃Cl₂NO₃ [M+H]⁺: 302, 304 (3:2) found 302, 304 (3:2); ¹H NMR (400 MHz, CD₃OD) δ 7.21 (d, J=8.8 Hz, 1H), 6.72 (d, J=8.8 Hz, 1H), 4.53-4.49 (m, 1H), 4.01 (dd, J=8.6, 5.7 Hz, 1H), 3.96-3.84 (m, 2H), 3.70-3.56 (m, 1H), 3.07 (td, J=13.0, 3.5 Hz, 1H), 2.53-2.38 (m, 2H), 1.83-1.73 (m, 1H), 1.61-1.51 (m, 1H).

### Example 92. Compound 253 ((6R,7aS)-6-(2,3-dichloro-6-hydroxyphenyl)-1-(hydroxymethyl)-tetrahydro-1H-pyrrolo[1,2-c][1,3]oxazol-3-one isomer 1) and Compound 254 ((6R,7aS)-6-(2,3-dichloro-6-hydroxyphenyl)-1-(hydroxymethyl)-tetrahydro-1H-pyrrolo[1,2-c][1,3]oxazol-3-one isomer 2)

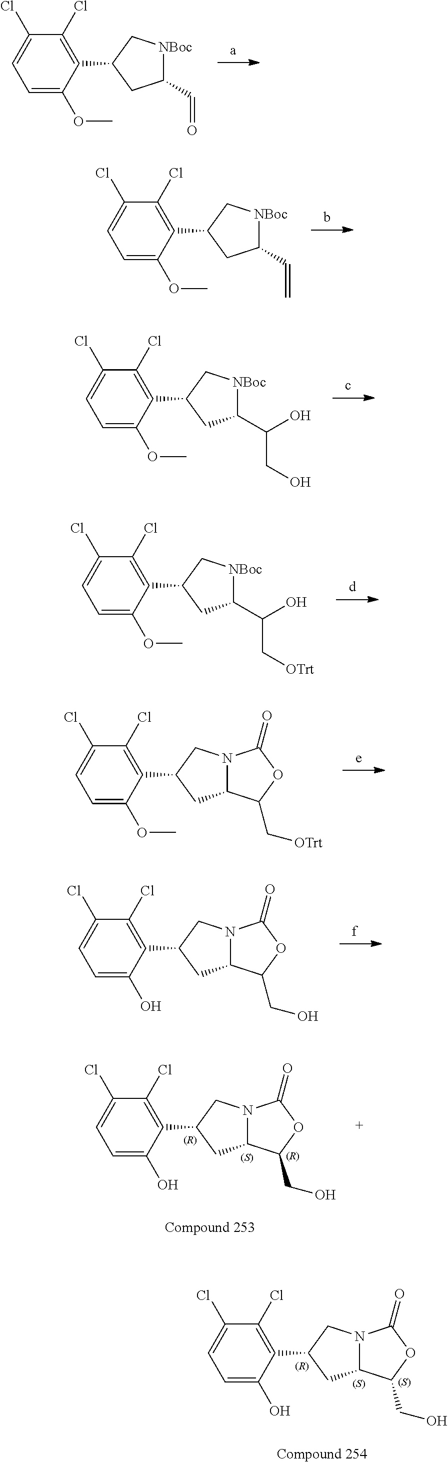

Step a:

To a stirred mixture of tert-butyl (2S,4R)-4-(2,3-dichloro-6-methoxyphenyl)-2-formylpyrrolidine-1-carboxylate (Example 7, step c) (0.480 g, 1.28 mmol) and methyltriphenylphosphonium bromide (0.920 g, 2.57 mmol) in THE (8 mL) was added t-BuOK (2.59 mL, 2.59 mmol, 1 M in THF) at 0° C. under nitrogen atmosphere. The reaction was stirred at 0° C. for 2 h, diluted with water (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by silica gel column chromatography, eluting with PE/EA (5/1) to afford tert-butyl (2S,4R)-4-(2,3-dichloro-6-methoxyphenyl)-2-ethenylpyrrolidine-1-carboxylate as an off-white solid (0.400 g, 84%): LCMS (ESI) calc'd for C₁₈H₂₃Cl₂NO₃ [M+H]⁺: 372, 374 (3:2) found 372, 374 (3:2); ¹H NMR (300 MHz, CDCl₃) δ 7.25 (d, J=8.9 Hz, 1H), 6.75 (d, J=8.9 Hz, 1H), 5.95-5.76 (m, 1H), 5.21-5.03 (m, 2H), 4.32 (q, J=7.9 Hz, 1H), 4.13-3.99 (m, 1H), 3.86-3.67 (m, 5H), 2.49-2.34 (m, 1H), 2.32-2.17 (m, 1H), 1.46 (s, 9H).

Step b:

To a stirred mixture of tert-butyl (2S,4R)-4-(2,3-dichloro-6-methoxyphenyl)-2-ethenylpyrrolidine-1-carboxylate (0.600 g, 1.61 mmol) in THE (3 mL), H₂O (3 mL) and acetone (3 mL) were added K₂OsO₄2H₂O (0.590 g, 1.61 mmol) and NMO (0.280 g, 2.42 mmol) at room temperature. The reaction was stirred for 1 h, diluted with water (30 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford tert-butyl (2S,4R)-4-(2,3-dichloro-6-methoxyphenyl)-2-(1,2-dihydroxyethyl)pyrrolidine-1-carboxylate as a black solid (0.560 g, 85%): LCMS (ESI) calc'd for C₁₈H₂₅Cl₂NO₅ [M+H]⁺: 406, 408 (3:2) found 406, 408 (3:2); ¹H NMR (300 MHz, CD₃OD) δ 7.41 (d, J=8.9 Hz, 1H), 6.99 (d, J=9.0 Hz, 1H), 4.22-4.07 (m, 1H), 4.07-3.92 (m, 2H), 3.92 (s, 3H), 3.84-3.40 (m, 4H), 2.64-2.47 (m, 1H), 2.24-2.10 (m, 1H), 1.51 (s, 9H).

Step c:

To a stirred solution of tert-butyl (2S,4R)-4-(2,3-dichloro-6-methoxyphenyl)-2-(1,2-dihydroxyethyl)pyrrolidine-1-carboxylate (0.600 g, 1.48 mmol) and triphenylmethyl chloride (1.23 g, 4.43 mmol) in DCM (6 mL) were added TEA (0.220 g, 2.22 mmol) and DMAP (54.0 mg, 0.44 mmol) at room temperature. The reaction was stirred for 16 h, diluted with water (30 mL) and extracted with EA (3×20 mL). The combined organic layers were washed with brine (3×20 mL) and dried over anhydrous Na₂SO₄. After filtration, the filtrate was concentrated under reduced pressure. The residue was purified by silica gel column chromatography, eluting with PE/EA (5/1) to afford tert-butyl (2S,4R)-4-(2,3-dichloro-6-methoxyphenyl)-2-[1-hydroxy-2-(triphenylmethoxy)ethyl]pyrrolidine-1-carboxylate as an off-white solid (0.420 g, 44%): LCMS (ESI) calc'd for C₃₇H₃₉Cl₂NO₅ [M+Na]⁺: 670, 672 (3:2) found 670, 672 (3:2); ¹H NMR (400 MHz, CD₃OD) δ 7.56-7.22 (m, 16H), 7.00-6.84 (m, 1H), 4.47-4.02 (m, 2H), 4.02-3.82 (m, 3H), 3.82-3.41 (m, 4H), 3.27-3.08 (m, 1H), 2.64-1.88 (m, 2H), 1.45 (s, 9H).

Step d:

A mixture of tert-butyl (2S,4R)-4-(2,3-dichloro-6-methoxyphenyl)-2-[1-hydroxy-2-(triphenylmethoxy)ethyl]pyrrolidine-1-carboxylate (0.430 g, 0.66 mmol) and NaH (31.0 mg, 60% in oil, 1.31 mmol) in DMF (5 mL) was stirred at 0° C. for 2 h under nitrogen atmosphere. The resulting mixture was quenched with water (20 mL) at 0° C. followed by extraction with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford (6R,7aS)-6-(2,3-dichloro-6-methoxyphenyl)-1-((trityloxy)methyl)tetrahydro-1H,3H-pyrrolo[1,2-c]oxazol-3-one as an off-white solid (0.180 g, 48%): LCMS (ESI) calc'd for C₃₃H₂₉Cl₂NO₄ [M+Na]⁺: 596, 598 (3:2) found 596, 598 (3:2); H NMR (300 MHz, CD₃OD) δ 7.52-7.22 (m, 16H), 6.92 (d, J=9.1 Hz, 1H), 5.06-4.93 (m, 1H), 4.41-4.18 (m, 2H), 3.83-3.67 (m, 1H), 3.66 (s, 3H), 3.58-3.37 (m, 2H), 3.30-3.15 (m, 1H), 1.90-1.67 (m, 2H).

Step e:

To a stirred mixture of (6R,7aS)-6-(2,3-dichloro-6-methoxyphenyl)-1-((trityloxy)methyl)tetrahydro-1H,3H-pyrrolo[1,2-c]oxazol-3-one (90.0 mg, 0.16 mmol) in DCM (2 mL) was added BBr₃ (0.10 mL, 1.06 mmol) at 0° C. The reaction was stirred at room temperature for 2 h and quenched with MeOH (2 mL). The residue was purified by Prep-HPLC with the following conditions: Column: X Select CSH Prep C18 OBD Column, 19×250 mm, 5 μm; Mobile Phase A: water (plus 0.05% TFA), Mobile Phase B: ACN; Flow rate: 20 mL/min; Gradient: 28% B to 40% B in 5.3 min; Detector: UV 254/210 nm; Retention time: 5.23 min. The fractions containing desired product were collected and concentrated under reduced pressure to afford (6R,7aS)-6-(2,3-dichloro-6-hydroxyphenyl)-1-(hydroxymethyl)-tetrahydro-1H-pyrrolo[1,2-c][1,3]oxazol-3-one as an off-white solid (22.5 mg, 45%): LCMS (ESI) calc'd for C₁₃H₁₃Cl₂NO₄ [M+H]⁺: 318, 320 (3:2) found 318, 320 (3:2): ¹H NMR (400 MHz, CD₃OD) δ 7.24 (dd, J=8.7, 1.8 Hz, 1H), 6.74 (dd, J=8.7, 1.8 Hz, 1H), 4.83-4.45 (m, 1H), 4.44-4.31 (m, 1H), 4.26-3.96 (m, 1H), 3.96-3.88 (m, 1H), 3.88-3.69 (m, 2H), 3.47-3.39 (m, 1H), 2.43-1.82 (m, 2H).

Step f:

The product (6R,7aS)-6-(2,3-dichloro-6-hydroxyphenyl)-1-(hydroxymethyl)-tetrahydro-1H-pyrrolo[1,2-c][1,3]oxazol-3-one (22.5 mg, 0.07 mmol) was separated by Prep Chiral HPLC with the following conditions: Column: CHIRALPAK ID, 2×25 cm, 5 μm; Mobile Phase A: Hex (plus 0.1% FA)-HPLC, Mobile Phase B: IPA-HPLC; Flow rate: 20 mL/min; Gradient: 10% B to 10% B in 19 min; Detector: UV 220/254 nm; Retention time 1: 13.49 min; Retention time 2: 15.78 min; Injection Volume: 0.3 mL; Number Of Runs: 7. The faster-eluting isomer at 13.49 min was obtained as Compound 253 ((6R,7aS)-6-(2,3-dichloro-6-hydroxyphenyl)-1-(hydroxymethyl)-tetrahydro-1H-pyrrolo[1,2-c][1,3]oxazol-3-one isomer 1) as an off-white solid (4.2 mg, 18%): LCMS (ESI) calc'd for C₁₃H₁₃Cl₂NO₄ [M+H]⁺: 318, 320 (3:2) found 318, 320 (3:2); ¹H NMR (300 MHz, CD₃OD) δ 7.24 (d, J=8.7 Hz, 1H), 6.74 (d, J=8.8 Hz, 1H), 4.83-4.75 (m, 1H), 4.44-4.18 (m, 2H), 4.00-3.72 (m, 3H), 3.45-3.37 (m, 1H), 2.40-2.38 (m, 1H), 1.93-1.81 (m, 1H). The slower-eluting isomer at 15.78 min was obtained Compound 254 ((6R,7aS)-6-(2,3-dichloro-6-hydroxyphenyl)-1-(hydroxymethyl)-tetrahydro-1H-pyrrolo[1,2-c][1,3]oxazol-3-one isomer 2) as an off-white solid (8.80 mg, 39%): LCMS (ESI) calc'd for C₁₃H₁₃Cl₂NO₄ [M+H]⁺: 318, 320 (3:2) found 318, 320 (3:2); ¹H NMR (300 MHz, CD₃OD) δ 7.24 (d, J=8.7 Hz, 1H), 6.74 (d, J=8.8 Hz, 1H), 4.54-4.47 (m, 1H), 4.47-4.26 (m, 1H), 4.04-3.86 (m, 2H), 3.86-3.65 (m, 2H), 3.45-3.40 (m, 1H), 2.32-2.16 (m, 2H).

### Example 93. Compound 255 ((6R,7aS)-6-(2,3-dichloro-6-hydroxyphenyl)-1-(2-hydroxypropan-2-yl)tetrahydro-1H,3H-pyrrolo[1,2-c]oxazol-3-one isomer 1), Compound 256 ((6R,7aS)-6-(2,3-dichloro-6-hydroxyphenyl)-1-(2-hydroxypropan-2-yl)tetrahydro-1H,3H-pyrrolo[1,2-c]oxazol-3-one isomer 2) and Compound 257 ((4aS,6R)-6-(2,3-dichloro-6-hydroxyphenyl)-4-hydroxy-3,3-dimethylhexahydro-1H-pyrrolo[1,2-c][1,3]oxazin-1-one)

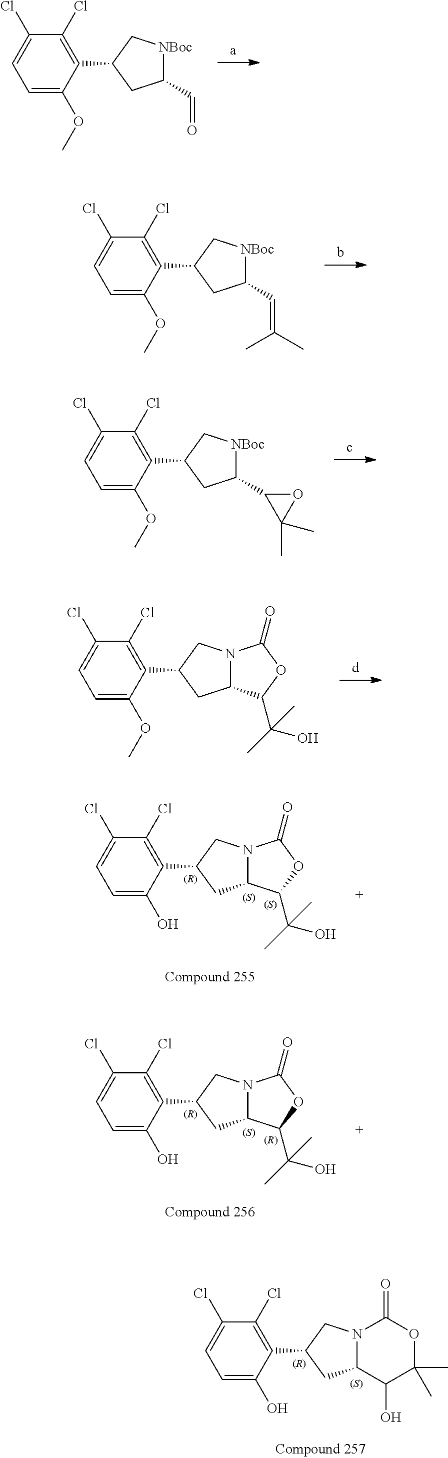

Step a:

To a stirred mixture of isopropyltriphenylphosphanium iodide (1.39 g, 3.215 mmol) in THE (10 mL) was added t-BuOK (3.21 mL, 3.21 mmol, 1 M solution in THF) at 0° C. under nitrogen atmosphere. The reaction was stirred at 0° C. for 10 min. and tert-butyl (2S,4R)-4-(2,3-dichloro-6-methoxyphenyl)-2-formylpyrrolidine-1-carboxylate (Example 7, step c) (0.600 g, 1.603 mmol) in THE (5 mL) was added. The reaction was stirred at 0° C. for 2 h, quenched with saturated aq. NH₄Cl (30 mL) at 0° C. and extracted with EA (3×30 mL). The combined organic layers were washed with brine (3×30 mL) and dried over anhydrous Na₂SO₄. After filtration, the filtrate was concentrated under reduced pressure. The residue was purified by silica gel column chromatography, eluting with PE/EA (3/1) to afford tert-butyl (2S,4R)-4-(2,3-dichloro-6-methoxyphenyl)-2-(2-methylprop-1-en-1-yl)pyrrolidine-1-carboxylate as a light yellow oil (0.400 g, 62%): LCMS (ESI) calc'd for C₂₀H₂₇Cl₂NO₃ [M+H]⁺: 400, 402 (3:2) found 400, 402 (3:2); ¹H NMR (300 MHz, CDCl₃) δ 7.32 (d, J=9.0 Hz, 1H), 6.76 (d, J=8.9 Hz, 1H), 5.32 (d, J=9.1 Hz, 1H) 4.77-4.72 (m, 1H), 4.39-4.23 (m, 1H), 3.84 (d, J=4.4 Hz, 3H), 3.82-3.68 (m, 1H), 3.63-3.59 (m, 1H), 3.48-3.42 (m, 1H), 2.82-2.68 (m, 1H), 1.78-1.72 (m, 6H), 1.47 (d, J=5.8 Hz, 9H).

Step b:

To a stirred solution of tert-butyl (2S,4R)-4-(2,3-dichloro-6-methoxyphenyl)-2-(2-methylprop-1-en-1-yl) pyrrolidine-1-carboxylate (0.200 g, 0.50 mmol) in DCM (2 mL) was added m-CPBA (0.250 g, 1.50 mmol) at room temperature. The reaction was stirred at room temperature for 2 h, quenched with saturated aq. Na₂SO₃ (20 mL) and extracted with EA (3×20 mL). The combined organic layers were washed with saturated aq. NaHCO₃ (3×20 mL) and dried over anhydrous Na₂SO₄. After filtration, the filtrate was concentrated under reduced pressure to afford tert-butyl (2S,4R)-4-(2,3-dichloro-6-methoxyphenyl)-2-(3,3-dimethyloxiran-2-yl) pyrrolidine-1-carboxylate as a yellow oil (0.100 g, crude): LCMS (ESI) calc'd for C₂₀H₂₇Cl₂NO₄ [M+H]⁺: 416, 418 (3:2) found 416, 418 (3:2).

Step c:

To a stirred solution of tert-butyl (2S,4R)-4-(2,3-dichloro-6-methoxyphenyl)-2-(3,3-dimethyloxiran-2-yl) pyrrolidine-1-carboxylate (0.100 g, 0.240 mmol) in MeOH (1 mL) was added TsOH H₂O (9.00 mg, 0.05 mmol) at room temperature. The reaction was stirred for 1 h and concentrated under reduced pressure. The residue was purified by reverse phase chromatography, eluting with 40% ACN in water (plus 0.05% TFA) solution to afford (6R,7aS)-6-(2,3-dichloro-6-methoxyphenyl)-1-(2-hydroxypropan-2-yl)-tetrahydro-1H-pyrrolo[1,2-c][1,3]oxazol-3-one as a yellow oil (40.0 mg, 46%): LCMS (ESI) calc'd for C₁₆H₁₉Cl₂NO₄ [M+H]⁺: 360, 362 (3:2) found 360, 362 (3:2).

Step d:

To a stirred solution of (6R,7aS)-6-(2,3-dichloro-6-methoxyphenyl)-1-(2-hydroxypropan-2-yl)-tetrahydro-1H-pyrrolo[1,2-c][1,3]oxazol-3-one (50.0 mg, 0.14 mmol) in DCM (1 mL) was added BBr₃ (0.13 mL, 0.52 mmol) at room temperature. The reaction was stirred for 1 h, quenched with MeOH (1 mL) and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 60% B to 80% B in 5.5 min; Detector: UV 220 nm; Retention time 1: 5.56 min; Retention time 2: 5.72 min; Retention time 3: 6.03 min. The faster-eluting isomer at 5.56 min was obtained as Compound 255 ((6R,7aS)-6-(2,3-dichloro-6-hydroxyphenyl)-1-(2-hydroxypropan-2-yl)-tetrahydro-1H-pyrrolo[1,2-c][1,3]oxazol-3-one isomer 1) as an off-white solid (4.6 mg, 9.57%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.25 (d, J=8.8 Hz, 1H), 6.77 (d, J=8.7 Hz, 1H), 4.34 (td, J=7.9, 2.7 Hz, 1H), 4.25-4.14 (m, 1H), 3.98 (dd, J=10.8, 8.9 Hz, 1H), 3.78 (dd, J=10.8, 7.9 Hz, 1H), 3.66 (d, J=2.7 Hz, 1H), 2.53-2.44 (m, 1H), 2.39-2.28 (m, 1H), 1.47 (s, 3H), 1.44 (s, 3H). The middle isomer at 5.72 min was obtained as Compound 256 ((6R,7aS)-6-(2,3-dichloro-6-hydroxyphenyl)-1-(2-hydroxypropan-2-yl)-tetrahydro-1H-pyrrolo[1,2-c][1,3]oxazol-3-one isomer 2) as an off-white solid (4.8 mg, 9.99%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.26 (d, J=8.8 Hz, 1H), 6.78 (d, J=8.8 Hz, 1H), 4.30-4.18 (m, 1H), 4.04 (dd, J=11.0, 9.1 Hz, 1H), 3.97-3.90 (m, 1H), 3.72 (dd, J=11.0, 7.4 Hz, 1H), 3.42 (d, J=9.6 Hz, 1H), 2.51-2.42 (m, 1H), 2.25-2.12 (m, 1H), 1.43 (s, 3H), 1.37 (s, 3H). The slower-eluting isomer at 6.03 min was obtained as Compound 257 ((4aS,6R)-6-(2,3-dichloro-6-hydroxyphenyl)-4-hydroxy-3,3-dimethyl-tetrahydro-4H-pyrrolo[1,2-c][1,3]oxazin-1-one) (4.6 mg) as an off-white solid (4.6 mg, 9.57%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.25 (dd, J=8.8, 2.2 Hz, 1H), 6.76 (d, J=8.8 Hz, 1H), 4.35-4.24 (m, 1H), 4.15-4.03 (m, 1H), 3.74-3.57 (m, 1H), 3.57-3.48 (m, 1H), 3.45 (d, J=9.3 Hz, 1H), 2.49-2.39 (m, 1H), 2.35-2.26 (m, 1H), 1.44 (d, J=3.0 Hz, 3H), 1.37 (s, 3H).

Example 94. Compounds 258-259 were prepared in an analogous fashion as that described for Compounds 255-257.

| Compound | | | |
|---|---|---|---|
| Number | Structure | Chemical Name | MS: (M + H)⁺ & ¹H MNR |
| 258 | 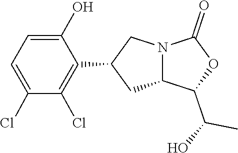 [SMILES: C[C@H](O)[C@H]1OC(=O)N2C[C@@H](c3c(O)ccc(Cl)c3Cl)C[C@@H]12] | (6R,7aS)-6-(2,3- dichloro-6- hydroxyphenyl)-1-[1- hydroxyethyl]- tetrahydro-1H- pyrrolo[1,2- c][1,3]oxazol-3-one isomer 1 | [M + H]⁺: 332, 334 (3:2); ¹H NMR (300 MHz, CDCl₃) δ 9.44 (s, 1H), 7.16 (d, J = 8.7 Hz, 1H), 6.83 (d, J = 8.7 Hz, 1H), 4.57-4.50 (m, 1H), 4.37- 4.22 (m, 1H), 4.20-4.06 (m, 2H), 4.00 (dd, J = 11.4, 5.8 Hz, 1H), 3.54-3.40 (m, 1H), 3.10 (s, 1H), 2.49-2.40 (m, 1H), 2.09-1.93 (m, 1H), 1.25 (d, J = 6.4 Hz, 3H). |
| 259 | 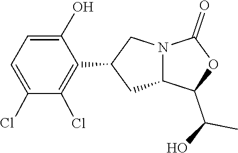 [SMILES: C[C@@H](O)[C@@H]1OC(=O)N2C[C@@H](c3c(O)ccc(Cl)c3Cl)C[C@@H]12] | (6R,7aS)-6-(2,3- dichloro-6- hydroxyphenyl)-1-[1- hydroxyethyl]- tetrahydro-1H- pyrrolo[1,2- c][1,3]oxazol-3-one isomer 2 | [M + H]⁺: 332, 334 (3:2); ¹H NMR (300 MHz, CDCl₃) δ 9.19 (s, 1H), 7.17 (dd, J = 8.7, 1.4 Hz, 1H), 6.85 (dd, J = 8.8, 1.4 Hz, 1H), 4.42-4.25 (m, 2H), 4.05-3.81 (m, 3H), 3.49-3.40 (m, 1H), 2.69 (s, 1H), 2.40-2.13 (m, 2H), 1.29 (d, J = 6.2 Hz, 3H). |

### Example 95. Compound 260 ((6R,7aS)-6-(2,3-dichloro-6-hydroxyphenyl)-1-(1,2-dihydroxyethyl)tetrahydro-1H,3H-pyrrolo[1,2-c]oxazol-3-one isomer 1), Compound 261 ((6R,7aS)-6-(2,3-dichloro-6-hydroxyphenyl)-1-(1,2-dihydroxyethyl)tetrahydro-1H,3H-pyrrolo[1,2-c]oxazol-3-one isomer 2), Compound 262 ((6R,7aS)-6-(2,3-dichloro-6-hydroxyphenyl)-1-(1,2-dihydroxyethyl)tetrahydro-1H,3H-pyrrolo[1,2-c]oxazol-3-one isomer 3) and Compound 263 ((6R,7aS)-6-(2,3-dichloro-6-hydroxyphenyl)-1-(1,2-dihydroxyethyl)tetrahydro-1H,3H-pyrrolo[1,2-c]oxazol-3-one isomer 4)

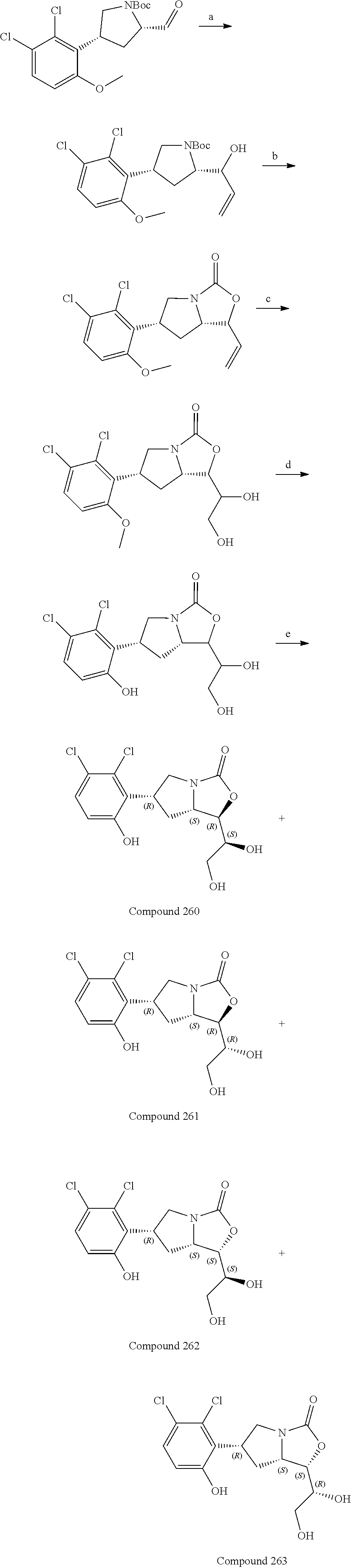

Step a

To a stirred solution of tert-butyl (2S,4R)-4-(2,3-dichloro-6-methoxyphenyl)-2-formylpyrrolidine-1- carboxylate (Example 7, step c) (1.00 g, 2.67 mmol) in THE (10 mL) was added vinylmagnesium bromide (5.34 mL, 5.34 mmol, 1 M solution in THF) at −65° C. under nitrogen atmosphere. The reaction was stirred at −65° C. for 2 h. The resulting mixture was quenched with saturated aq. NH₄Cl (20 mL) at room temperature followed by extraction with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 65% ACN in water (plus 10 mM NH₄HCO₃) to afford tert-butyl (2S,4R)-4-(2,3-dichloro-6-methoxyphenyl)-2-(1-hydroxyprop-2-en-1-yl)pyrrolidine-1-carboxylate as a yellow oil (0.800 g, 74%): LCMS (ESI) calc'd for C₁₉H₂₅Cl₂NO₄ [M+H]⁺: 402, 404 (3:2) found 402, 404 (3:2); ¹H NMR (400 MHz, CDCl₃) δ 7.38-7.31 (m, 1H), 6.81-6.74 (m, 1H), 6.04-5.11 (m, 3H), 4.39-4.11 (m, 2H), 4.11-3.89 (m, 1H), 3.89-3.62 (m, 5H), 2.73-1.87 (m, 2H), 1.56-1.32 (m, 9H).

Step b:

To a stirred solution of tert-butyl (2S,4R)-4-(2,3-dichloro-6-methoxyphenyl)-2-(1-hydroxyprop-2-en-1-yl) pyrrolidine-1-carboxylate (0.800 g, 1.99 mmol) in DMF (8 mL) was added NaH (0.100 g, 60% in oil, 3.98 mmol) at 0° C. under nitrogen atmosphere. The reaction was stirred at room temperature for 16 h. The resulting mixture was quenched with water (50 mL) at room temperature followed by extraction with EA (3×30 mL). The combined organic layers were washed with brine (5×30 mL) and dried over anhydrous Na₂SO₄. After filtration, the filtrate was concentrated under reduced pressure. The residue was purified by reverse phase chromatography, eluting with 60% ACN in water (plus 0.05% TFA) to afford (6R,7aS)-6-(2,3-dichloro-6-methoxyphenyl)-1-ethenyl-tetrahydro-1H-pyrrolo[1,2-c][1,3] oxazol-3-one as a yellow oil (0.330 g, 51%): LCMS (ESI) calc'd for C₁₅H₁₅Cl₂NO₃ [M+H]⁺: 328, 330 (3:2) found 328, 330 (3:2); ¹H NMR (400 MHz, CDCl₃) δ 7.34 (d, J=8.9 Hz, 1H), 6.78 (d, J=8.9 Hz, 1H), 6.08-5.81 (m, 1H), 5.58-5.43 (m, 1H), 5.39-5.31 (m, 1H), 5.23-4.78 (m, 1H), 4.40-4.05 (m, 1H), 4.00-3.78 (m, 5H), 3.47-3.38 (m, 1H), 2.28-1.77 (m, 2H).

Step c:

To a stirred solution of (6R,7aS)-6-(2,3-dichloro-6-methoxyphenyl)-1-ethenyl-tetrahydro-1H-pyrrolo[1,2-c] [1,3]oxazol-3-one (0.200 g, 0.61 mmol) in THE (1 mL), acetone (1 mL) and H₂O (1 mL) were added NMO (0.140 g, 1.22 mmol,) and K₂OsO₄. 2H₂O (0.110 g, 0.31 mmol) at room temperature. The reaction was stirred for 1 h, quenched with saturated aq. Na₂S₂O₃ (1 mL) and concentrated under reduced pressure. The residue was purified by reverse phase chromatography, eluting with 50% ACN in water (plus 0.05% TFA) to afford (6R,7aS)-6-(2,3-dichloro-6-methoxyphenyl)-1-(1,2-dihydroxyethyl)-tetrahydro-1H-pyrrolo[1,2-c][1,3]oxazol-3-one as a yellow oil (0.180 g, 62%): LCMS (ESI) calc'd for C₁₅H₁₇Cl₂NO₅ [M+H]⁺: 362, 364 (3:2) found 362, 364 (3:2); ¹H NMR (300 MHz, CDCl₃) δ 7.33 (d, J=8.9 Hz, 1H), 6.80-6.71 (m, 1H), 4.69-4.45 (m, 1H), 4.43-4.21 (m, 1H), 4.19-3.85 (m, 4H), 3.85-3.79 (m, 4H), 3.47-3.34 (m, 1H), 2.38 (s, 2H), 2.29-1.97 (m, 2H).

Step d:

To a stirred solution of (6R,7aS)-6-(2,3-dichloro-6-methoxyphenyl)-1-(1,2-dihydroxyethyl)-tetrahydro-1H-pyrrolo[1,2-c] [1,3]oxazol-3-one (0.180 g, 0.50 mmol) in DCM (2 mL) was added BBr₃ (0.47 mL, 4.97 mmol) at room temperature. The reaction was stirred for 1 h, quenched with MeOH (1 mL) and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 35% to 65% in 6.5 min; Detector: UV 254/220 nm; Retention time: 6.54 min. The fractions containing the desired product were collected and concentrated under reduced pressure to afford (6R,7aS)-6-(2,3-dichloro-6-hydroxyphenyl)-1-(1,2-dihydroxyethyl)-tetrahydro-1H-pyrrolo[1,2-c][1,3]oxazol-3-one as an off-white solid (43.1 mg, 24.9%): LCMS (ESI) calc'd for C₁₄H₁₅Cl₂NO₅ [M+H]⁺: 348, 350 (3:2) found 348, 350 (3:2); ¹H NMR (400 MHz, CD₃OD) δ 7.24 (d, J=8.8 Hz, 1H), 6.74 (d, J=8.8 Hz, 1H), 4.75-4.56 (m, 1H), 4.50-4.31 (m, 1H), 4.27-4.11 (m, 1H), 4.01-3.86 (m, 1H), 3.84-3.58 (m, 3H), 3.48-3.38 (m, 1H), 2.52-1.97 (m, 2H).

Step e:

The product (6R,7aS)-6-(2,3-dichloro-6-hydroxyphenyl)-1-(1,2-dihydroxyethyl)-tetrahydro-1H-pyrrolo [1,2-c][1,3]oxazol-3-one (40.0 mg, 0.12 mmol) was separated by Prep Chiral HPLC with the following conditions: Column: CHIRALPAK IF, 2×25 cm, 5 um; Mobile Phase A: Hex (plus 0.1% FA)-HPLC, Mobile Phase B: EtOH-HPLC; Flow rate: 20 mL/min; Gradient: 20% B to 20% B in 12 min; Detector: UV 254/220 nm; Retention time 1: 8.16 min 1; Retention time 2: 10.67 min. The faster-eluting isomer at 8.16 min was obtained (1S,6R,7aS)-6-(2,3-dichloro-6-hydroxyphenyl)-1-(1,2-dihydroxyethyl)-tetrahydro-1H-pyrrolo[1,2-c][1,3]oxazol-3-one as an off-white solid (12.7 mg, 31.8%). The slower-eluting isomer at 10.67 min was obtained (1R,6R,7aS)-6-(2,3-dichloro-6-hydroxyphenyl)-1-(1,2-dihydroxyethyl)-tetrahydro-1H-pyrrolo[1,2-c][1,3]oxazol-3-one as an off-white solid (16.8 mg, 42.0%).

The product (6R,7aS)-6-(2,3-dichloro-6-hydroxyphenyl)-1-(1,2-dihydroxyethyl)-tetrahydro-1H-pyrrolo[1,2-c][1,3]oxazol-3-one isomer 1 (12.7 mg) was separated by Prep Chiral HPLC with the following conditions: Column: CHIRALPAK IC, 2×25 cm, 5 μm; Mobile Phase A: Hex (plus 0.1% FA)-HPLC, Mobile Phase B: EtOH-HPLC; Flow rate: 20 mL/min; Gradient: 20% to 20% in 10 min; Detector: UV 254/220 nm; Retention time 1: 6.03 min; Retention time 2: 8.65 min. The faster-eluting isomer at 6.03 min was obtained as Compound 260 ((6R,7aS)-6-(2,3-dichloro-6-hydroxyphenyl)-1-[1,2-dihydroxyethyl]-tetrahydro-1H-pyrrolo[1,2-c][1,3]oxazol-3-one isomer 1) as an off-white solid (8.5 mg, 21%): LCMS (ESI) calc'd for C₁₄H₁₅Cl₂NO₅ [M+H]⁺: 348, 350 (3:2) found 348, 350 (3:2); ¹H NMR (400 MHz, CD₃OD) δ 7.24 (d, J=8.8 Hz, 1H), 6.74 (d, J=8.8 Hz, 1H), 4.47 (dd, J=5.9, 3.8 Hz, 1H), 4.45-4.35 (m, 1H), 4.24-4.16 (m, 1H), 3.91 (dd, J=10.7, 7.0 Hz, 1H), 3.85-3.80 (m, 1H), 3.73-3.62 (m, 2H), 3.45-3.40 (m, 1H), 2.31-2.14 (m, 2H). The slower-eluting isomer at 8.65 min was obtained as Compound 261 ((6R,7aS)-6-(2,3-dichloro-6-hydroxyphenyl)-1-[1,2-dihydroxyethyl]-tetrahydro-1H-pyrrolo[1,2-c][1,3]oxazol-3-one isomer 2) as an off-white solid (1.8 mg, 4.5%): LCMS (ESI) calc'd for C₁₄H₁₅Cl₂NO₅ [M+H]⁺: 348, 350 (3:2) found 348, 350 (3:2); ¹H NMR (400 MHz, CD₃OD) δ 7.24 (d, J=8.8 Hz, 1H), 6.74 (d, J=8.8 Hz, 1H), 4.62-4.58 (m, 1H), 4.46-4.34 (m, 1H), 4.16-4.08 (m, 1H), 3.96-3.87 (m, 1H), 3.78-3.70 (m, 1H), 3.68 (d, J=6.8 Hz, 2H), 3.45-3.40 (m, 1H), 2.31-2.15 (m, 2H).

The product (6R,7aS)-6-(2,3-dichloro-6-hydroxyphenyl)-1-(1,2-dihydroxyethyl)-tetrahydro-1H-pyrrolo[1,2-c][1,3]oxazol-3-one isomer 2 (16.8 mg) was separated by Prep Chiral HPLC with the following conditions: Column: CHIRALPAK IC, 2×25 cm, 5 μm; Mobile Phase A: Hex (plus 0.1% FA)-HPLC, Mobile Phase B: EtOH-HPLC; Flow rate: 20 mL/min; Gradient: 20% to 20% in 10 min; Detector: UV 254/220 nm; Retention time 1: 5.75 min; Retention time 2: 8.06 min. The faster-eluting isomer at 5.75 min was obtained: as Compound 262 ((6R,7aS)-6-(2,3-dichloro-6-hydroxyphenyl)-1-[1,2-dihydroxyethyl]-tetrahydro-1H-pyrrolo[1,2-c][1,3]oxazol-3-one isomer 3) as an off-white solid. (12.2 mg, 30.5%): LCMS (ESI) calc'd for C₁₄H₁₅Cl₂NO₅ [M+H]⁺: 348, 350 (3:2) found 348, 350 (3:2); ¹H NMR (400 MHz, CD₃OD) δ 7.24 (d, J=8.8 Hz, 1H), 6.73 (d, J=8.7 Hz, 1H), 4.67 (dd, J=9.2, 7.7 Hz, 1H), 4.41-4.30 (m, 1H), 4.27-4.16 (m, 1H), 3.96 (dd, J=10.7, 7.3 Hz, 1H), 3.82-3.75 (m, 2H), 3.66 (dd, J=12.3, 5.8 Hz, 1H), 3.45-3.40 (m, 1H), 2.49-2.43 (m, 1H), 2.08-1.98 (m, 1H). The slower-eluting isomer at 8.06 min was obtained as Compound 263 ((6R,7aS)-6-(2,3-dichloro-6-hydroxyphenyl)-1-[1,2-dihydroxyethyl]-tetrahydro-1H-pyrrolo[1,2-c][1,3]oxazol-3-one isomer 4) as an off-white solid (1.2 mg, 3%): LCMS (ESI) calc'd for C₁₄H₁₅Cl₂NO₅ [M+H]⁺: 348, 350 (3:2) found 348, 350 (3:2); ¹H NMR (400 MHz, CD₃OD) δ 7.24 (d, J=8.8 Hz, 1H), 6.73 (d, J=8.8 Hz, 1H), 4.75-4.70 (m, 1H), 4.40-4.29 (m, 1H), 4.20-4.11 (m, 1H), 3.99 (dd, J=10.6, 7.7 Hz, 1H), 3.89-3.81 (m, 1H), 3.69-3.58 (m, 2H), 3.42-3.36 (m, 1H), 2.57-2.50 (m, 1H), 1.93-1.84 (m, 1H).

### Example 96. Compound 264 ((6R,7aS)-6-(2,3-dichloro-6-hydroxyphenyl)-1-(hydroxymethyl)-1-methyltetrahydro-1H,3H-pyrrolo[1,2-c]oxazol-3-one isomer 1) and Compound 265 (((6R,7aS)-6-(2,3-dichloro-6-hydroxyphenyl)-1-(hydroxymethyl)-1-methyltetrahydro-1H,3H-pyrrolo[1,2-c]oxazol-3-one isomer 2)

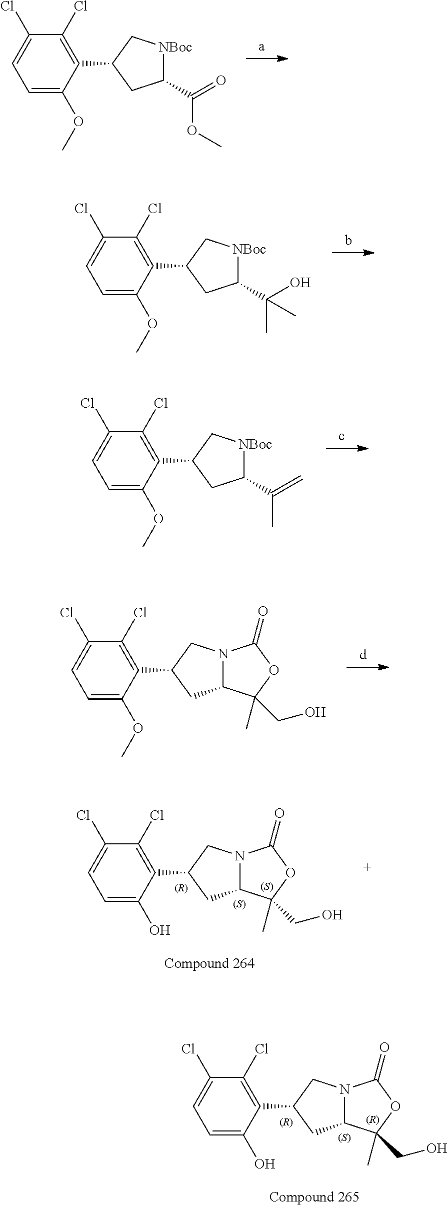

Step a:

To a stirred solution of 1-tert-butyl 2-methyl (2S,4R)-4-(2,3-dichloro-6-methoxyphenyl) pyrrolidine-1,2- dicarboxylate (2.00 g, 4.95 mmol) in THE (20 mL) was added MeMgCl (8.25 mL, 24.8 mmol, 3 M in THF) at 0° C. under nitrogen atmosphere. The reaction was stirred at room temperature for 2 h, quenched with saturated aq. NH₄Cl (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by silica gel column chromatography, eluting with PE/EA (2/1) to afford tert-butyl (2S,4R)-4-(2,3-dichloro-6- methoxyphenyl)-2-(2-hydroxypropan-2-yl)pyrrolidine-1-carboxylate as a colorless oil (1.40 g, 70%): LCMS (ESI) calc'd for C₁₉H₂₇Cl₂NO₄ [M+H]⁺: 404, 406 (3:2) found 404, 406 (3:2); ¹H NMR (400 MHz, CDCl₃) δ 7.34 (d, J=8.9 Hz, 1H), 6.77 (d, J=8.9 Hz, 1H), 4.04 (dd, J=9.7, 7.8 Hz, 1H), 3.98-3.87 (m, 1H), 3.85 (s, 3H), 3.82-3.73 (m, 2H), 2.41-2.31 (m, 1H), 2.22-2.08 (m, 1H), 1.49 (s, 9H), 1.22 (s, 3H), 1.16 (s, 3H).

Step b:

To a stirred mixture of tert-butyl (2S,4R)-4-(2,3-dichloro-6-methoxyphenyl)-2-(2-hydroxypropan-2-yl) pyrrolidine-1-carboxylate (0.300 g, 0.74 mmol) in THF (4 mL) was added SOCl₂ (0.12 mL, 1.01 mmol) in portions at −78° C. under nitrogen atmosphere. The reaction was stirred at −78° C. for 1 h. To the above mixture was added TEA (1.03 mL, 10.18 mmol) at −78° C. The reaction was stirred at −78° C. to room temperature for an additional 16 h. The resulting mixture was diluted by water (10 mL) and extracted with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 80% ACN in water (plus 0.05% TFA) to afford tert-butyl (2S,4R)-4-(2,3-dichloro-6-methoxyphenyl)-2-(prop-1-en-2-yl)pyrrolidine-1-carboxylate as a brown oil (0.13 g, 45%): LCMS (ESI) calc'd for C₁₉H₂₅Cl₂NO₃ [M+H]⁺: 386, 388 (3:2) found 386, 388 (3:2); ¹H NMR (400 MHz, CDCl₃) δ 7.34 (d, J=8.9 Hz, 1H), 6.76 (d, J=9.0 Hz, 1H), 5.06-4.65 (m, 2H), 4.50-4.20 (m, 1H), 4.20-3.92 (m, 1H), 3.92-3.53 (m, 5H), 2.59-2.40 (m, 1H), 2.26-2.09 (m, 1H), 1.76 (s, 3H), 1.47 (s, 9H).

Step c:

To a stirred mixture of tert-butyl (2S,4R)-4-(2,3-dichloro-6-methoxyphenyl)-2-(prop-1-en-2-yl) pyrrolidine-1-carboxylate (0.130 g, 0.44 mmol,) in DCM (2 mL) was added m-CPBA (0.220 g, 1.32 mmol) at room temperature. The reaction was stirred at room temperature for 2 h, quenched with saturated aq. Na₂SO₃ (20 mL) and extracted with EA (3×20 mL). The combined organic layers were washed with saturated aq. NaHCO₃ (3×20 mL) and brine and then dried over anhydrous Na₂SO₄. After filtration, the filtrate was concentrated under reduced pressure. The residue was purified by reverse phase chromatography, eluting with 40% ACN in water (plus 0.05% TFA) to afford (6R,7aS)-6-(2,3-dichloro-6-methoxyphenyl)-1-(hydroxymethyl)-1-methyl-tetrahydropyrrolo[1,2-c][1,3]oxazol-3-one as a yellow oil (60.0 mg, 39%): LCMS (ESI) calc'd for C₁₅H₁₇Cl₂NO₄ [M+H]⁺: 346, 348 (3:2) found 346, 348 (3:2): ¹HNMR (400 MHz, CDCl₃) δ 7.35 (dd, J=9.0, 3.6 Hz, 1H), 6.79 (dd, J=8.9, 7.3 Hz, 1H), 4.50 (s, 1H), 4.40-4.30 (m, 1H), 3.99-3.91 (m, 2H), 3.88-3.83 (m, 4H), 3.83-3.69 (m, 1H), 3.49-3.35 (m, 1H), 2.53-1.87 (m, 2H), 1.52 (d, J=66.6 Hz, 3H).

Step d:

To a stirred solution of (6R,7aS)-6-(2,3-dichloro-6-methoxyphenyl)-1-(hydroxymethyl)-1-methyl-tetrahydropyrrolo[1,2-c][1,3]oxazol-3-one (60.0 mg, 0.17 mmol) in DCM (1 mL) was added BBr₃ (0.16 mL, 1.69 mmol) at room temperature. The reaction was stirred for 1 h, quenched with MeOH (1 mL) and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 35% to 40% in 6.5 min; Detector: UV 254/220 nm; Retention time 1: 6.57 min; Retention time 2: 6.78 min. The faster-eluting isomer at 6.57 min was obtained as Compound 264 ((6R,7aS)-6-(2,3-dichloro-6-hydroxyphenyl)-1-(hydroxymethyl)-1-methyl-tetrahydropyrrolo[1,2-c][1,3]oxazol-3-one isomer 1) as an off-white solid (7.40 mg, 12.9%): LCMS (ESI) calc'd for C₁₄H₁₅Cl₂NO₄ [M+H]⁺: 332, 334 (3:2) found 332, 334 (3:2); ¹H NMR (400 MHz, CD₃OD) δ 7.25 (d, J=8.8 Hz, 1H), 6.75 (d, J=8.8 Hz, 1H), 4.44-4.33 (m, 1H), 4.04 (dd, J=11.1, 5.8 Hz, 1H), 3.97 (dd, J=10.6, 7.5 Hz, 1H), 3.65 (s, 2H), 3.43-3.38 (m, 1H), 2.49-2.40 (m, 1H), 1.94-1.83 (m, 1H), 1.40 (s, 3H). The slower-eluting isomer at 6.78 min was obtained as Compound 265 ((6R,7aS)-6-(2,3-dichloro-6-hydroxyphenyl)-1-(hydroxymethyl)-1-methyl-tetrahydropyrrolo[1,2-c][1,3]oxazol-3-one isomer 2) as an off-white solid (8.90 mg, 15.5%): LCMS (ESI) calc'd for C₁₄H₁₅Cl₂NO₄ [M+H]⁺: 332, 334 (3:2) found 332, 334 (3:2); ¹H NMR (400 MHz, CD₃OD) δ 7.24 (d, J=8.8 Hz, 1H), 6.74 (d, J=8.7 Hz, 1H), 4.42-4.28 (m, 1H), 3.95 (dd, J=10.6, 7.6 Hz, 1H), 3.88 (dd, J=11.1, 5.6 Hz, 1H), 3.70 (s, 2H), 3.40-3.36 (m, 1H), 2.46-2.40 (m, 1H), 1.99-1.90 (m, 1H), 1.58 (s, 3H).

### Example 97. Compound 266 ((4aS,6R)-6-(2,3-dichloro-6-hydroxyphenyl)-4-hydroxy-hexahydropyrrolo[1,2-c][1,3]oxazin-1-one isomer 1) and Compound 267 ((4aS,6R)-6-(2,3-dichloro-6-hydroxyphenyl)-4-hydroxy-hexahydropyrrolo[1,2-c][1,3]oxazin-1-one isomer 2)

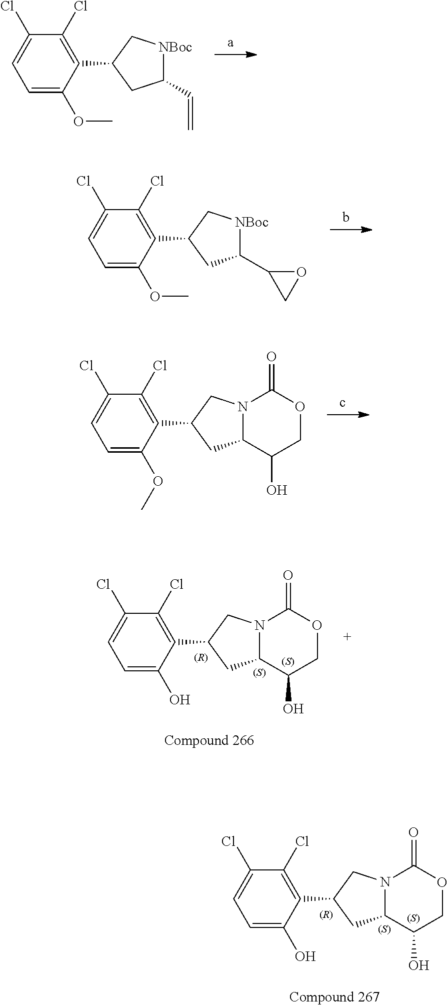

Step a:

To a stirred mixture of tert-butyl (2S,4R)-4-(2,3-dichloro-6-methoxyphenyl)-2-ethenylpyrrolidine-1-carboxylate (Example 14, step a) (50.0 mg, 0.13 mmol) in DCM (1 mL) was added m-CPBA (70 mg, 0.40 mmol) at room temperature. The reaction was stirred at room temperature for 2 h, quenched with saturated aq. Na₂SO₃ (20 mL) and extracted with EA (3×20 mL). The combined organic layers were washed with saturated aq. NaHCO₃ (2×20 mL), and brine (2×20 mL) and dried over anhydrous Na₂SO₄. After filtration, the filtrate was concentrated under reduced pressure to afford tert-butyl (2S,4R)-4-(2,3-dichloro-6-methoxyphenyl)-2-(oxiran-2-yl)pyrrolidine-1-carboxylate as an off-white solid (80.0 mg, crude), which was used in the next step directly without purification: LCMS (ESI) calc'd for C₁₈H₂₃Cl₂NO₄ [M+H−56]⁺: 332, 334 (3:2) found 332, 334 (3:2).

Step b:

To a stirred mixture of tert-butyl (2S,4R)-4-(2,3-dichloro-6-methoxyphenyl)-2-(oxiran-2-yl)pyrrolidine-1-carboxylate (50.0 mg, 0.13 mmol) in MeOH (1 mL) was added TsOH (4 mg, 0.03 mmol) at room temperature. The reaction was stirred for 3 h and concentrated under reduced pressure. The residue was purified by reverse phase chromatography, eluting with 50% ACN in water (plus 0.05% TFA) to afford (4aS,6R)-6-(2,3-dichloro-6-hydroxyphenyl)-4-hydroxyhexahydro-1H-pyrrolo[1,2-c][1,3]oxazin-1-one as a light yellow oil (14.0 mg, 36%): LCMS (ESI) calc'd for C₁₄H₁₅Cl₂NO₄ [M+H]⁺: 332, 334 (3:2), found 332, 334 (3:2); ¹H NMR (400 MHz, CD₃OD) δ 7.41 (d, J=9.0 Hz, 1H), 6.99 (d, J=8.9 Hz, 1H), 4.83-4.47 (m, 1H), 4.47-4.33 (m, 1H), 4.24-3.93 (m, 1H), 3.84 (d, J=3.7 Hz, 3H), 3.83-3.71 (m, 3H), 3.48-3.36 (m, 1H), 2.30-1.86 (m, 2H).

Step c:

To a stirred mixture of (4aS,6R)-6-(2,3-dichloro-6-methoxyphenyl)-4-hydroxy-hexahydropyrrolo[1,2-c][1,3]oxazin-1-one (14.0 mg, 0.04 mmol) in DCM (1 mL) was added BBr₃ (0.01 mL, 0.03 mmol) at room temperature. The reaction was stirred at room temperature for 1 h, quenched with MeOH (2 mL) and concentrated under reduced pressure. The residue was purified by Prep-HPLC with the following conditions: Column: X Select CSH Prep C18 OBD Column, 19×250 mm, 5 μm; Mobile Phase A: water (plus 0.05% TFA), Mobile Phase B: ACN; Flow rate: 20 mL/min; Gradient: 30% B to 40% B in 5.5 min; Detector: UV 254/210 nm; Retention time 1: 5.56 min, Retention time 2: 5.85 min. The faster-eluting isomer at 5.56 min was obtained as Compound 266 ((4aS,6R)-6-(2,3-dichloro-6-hydroxyphenyl)-4-hydroxy-hexahydropyrrolo[1,2-c][1,3]oxazin-1-one isomer 1) as an off-white solid (0.600 mg, 4.47%): LCMS (ESI) calc'd for C₁₃H₁₃Cl₂NO₄ [M+H]⁺: 318, 320 (3:2) found 318, 320 (3:2); ¹H NMR (400 MHz, CD₃OD) δ 7.25 (d, J=8.8 Hz, 1H), 6.76 (d, J=8.8 Hz, 1H), 4.42-4.31 (m, 2H), 4.30-4.17 (m, 1H), 4.14-4.05 (m, 2H), 3.97-3.88 (m, 1H), 3.53-3.49 (m, 1H), 3.02-2.96 (m, 1H), 1.97-1.85 (m, 1H). The slower-eluting isomer at 5.85 min was obtained as Compound 267 ((4aS,6R)-6-(2,3-dichloro-6-hydroxyphenyl)-4-hydroxy-hexahydropyrrolo[1,2-c][1,3]oxazin-1-one isomer 2) as an off-white solid (1.20 mg, 8.95%): LCMS (ESI) calc'd for C₁₃H₁₃Cl₂NO₄ [M+H]⁺: 318, 320 (3:2) found 318, 320 (3:2); ¹H NMR (400 MHz, CD₃OD) δ 7.26 (d, J=8.8 Hz, 1H), 6.76 (d, J=8.8 Hz, 1H), 4.32-4.19 (m, 2H), 4.11-3.97 (m, 2H), 3.87-3.74 (m, 1H), 3.64-3.49 (m, 2H), 2.49-2.43 (m, 1H), 2.36-2.29 (m, 1H).

### Example 98. Compound 268 ((4aS,6R)-6-(2,3-dichloro-6-hydroxyphenyl)-3-(hydroxymethyl)hexahydro-1H-pyrrolo[1,2-c][1,3]oxazin-1-one isomer 1) and Compound 269 ((4aS,6R)-6-(2,3-dichloro-6-hydroxyphenyl)-3-(hydroxymethyl)hexahydro-1H-pyrrolo[1,2-c][1,3]oxazin-1-one isomer 2)

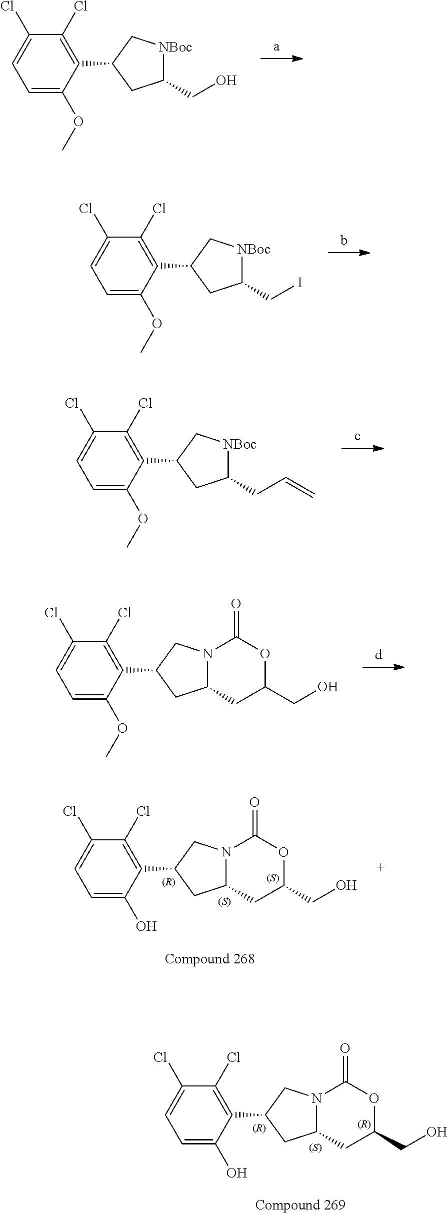

Step a:

To a stirred solution of tert-butyl (2S,4R)-4-(2,3-dichloro-6-methoxyphenyl)-2-(hydroxymethyl)pyrrolidine-1-carboxylate (Example 7, step b) (2.00 g, 5.32 mmol), PPh₃ (2.79 g, 10.64 mmol) and 12 (1.35 g, 5.32 mmol) in dry THE (15 mL) was added DEAD (1.67 mL, 9.58 mmol) dropwise at 0° C. under nitrogen atmosphere. The reaction was stirred at room temperature for 12 h and concentrated under reduced pressure. The residue was purified by silica gel column chromatography, eluting with EA/PE (1/3) to afford tert-butyl (2S,4R)-4-(2,3-dichloro-6-methoxyphenyl)-2-(iodomethyl)pyrrolidine-1-carboxylate as an off-white solid (0.95 g, 37%): LCMS (ESI) calc'd for C₁₇H₂₂Cl₂INO₃ [M+H]⁺: 486, 488 (3:2) found 486, 488 (3:2); ¹H NMR (300 MHz, CDCl₃) δ 7.33 (d, J=8.9 Hz, 1H), 6.77 (d, J=8.9 Hz, 1H), 4.17-4.00 (m, 1H), 3.93-3.71 (m, 6H), 3.65-3.49 (m, 2H), 2.64-2.45 (m, 1H), 2.33-2.18 (m, 1H), 1.50 (s, 9H).

Step b:

To a stirred solution of tert-butyl (2S,4R)-4-(2,3-dichloro-6-methoxyphenyl)-2-(iodomethyl)pyrrolidine-1-carboxylate (0.800 g, 1.65 mmol,), CuI (0.620 g, 3.29 mmol) in THE (8 mL) was added vinylmagnesium bromide (5.27 mL, 5.27 mmol, 1 M in THF) dropwise at −78° C. under nitrogen atmosphere. The reaction was stirred at −30° C. for additional 3 h, quenched with saturated aq. NH₄Cl (30 mL) and extracted with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 70% ACN in water (plus 0.05% TFA) to afford tert-butyl (2R,4R)-4-(2,3-dichloro-6-methoxyphenyl)-2-(prop-2-en-1-yl)pyrrolidine-1-carboxylate as an off-white solid (0.550 g, 87%): LCMS (ESI) calc'd for C₁₉H₂₅Cl₂NO₃ [M+H]⁺: 386, 388 (3:2) found 386, 388 (3:2); ¹H NMR (300 MHz, CDCl₃) δ 7.36 (d, J=8.9 Hz, 1H), 6.80 (d, J=8.6 Hz, 1H), 5.97-5.71 (m, 1H), 5.14 (dd, J=14.0, 8.0 Hz, 2H), 4.23-3.92 (m, 2H), 3.91-3.65 (m, 5H), 2.83-2.57 (m, 1H), 2.57-2.34 (m, 1H), 2.26-2.02 (m, 2H), 1.53 (s, 9H).

Step c:

To a stirred solution of tert-butyl (2R,4R)-4-(2,3-dichloro-6-methoxyphenyl)-2-(prop-2-en-1-yl)pyrrolidine-1-carboxylate (0.200 g, 0.52 mmol,) in DCM (2 mL) was added m-CPBA (0.270 g, 1.55 mmol) at room temperature. The reaction was stirred for 2 h, quenched with saturated aq. Na₂SO₃ (20 mL) and extracted with EA (3×20 mL). The combined organic layers were washed with saturated aq. NaHCO₃ (2×20 mL), brine (2×20 mL) and dried over anhydrous Na₂SO₄. After filtration, the filtrate was concentrated under reduced pressure. The residue was purified by reverse phase chromatography, eluting with 45% ACN in water (plus 0.05% TFA) to afford (4aS,6R)-6-(2,3-dichloro-6-methoxyphenyl)-3-(hydroxymethyl)-hexahydropyrrolo[1,2-c][1,3]oxazin-1-one as an off-white solid (0.110 g, 61%): LCMS (ESI) calc'd for C₁₅H₁₇Cl₂NO₄ [M+H]⁺: 346, 348 (3:2) found 346, 348 (3:2); ¹H NMR (400 MHz, CD₃OD) δ 7.42 (d, J=9.0 Hz, 1H), 7.00 (d, J=9.0 Hz, 1H), 4.56-4.23 (m, 2H), 4.02-3.95 (m, 1H), 3.93-3.81 (m, 4H), 3.81-3.66 (m, 2H), 3.54 (td, J=10.3, 3.3 Hz, 1H), 2.46-2.28 (m, 1H), 2.28-2.11 (m, 2H), 1.92-1.57 (m, 1H).

Step d:

To a stirred solution of (4aS,6R)-6-(2,3-dichloro-6-methoxyphenyl)-3-(hydroxymethyl)-hexahydropyrrolo [1,2-c][1,3]oxazin-1-one (0.11 g, 0.32 mmol) in DCM (2 mL) was added BBr₃ (0.21 mL, 0.84 mmol) dropwise at room temperature. The reaction was stirred for 1 h, quenched with MeOH (2 mL) and concentrated under reduced pressure. The residue was purified by Prep-HPLC with the following conditions: Column: X Select CSH Prep C18 OBD Column, 5 μm, 19×150 mm; Mobile Phase A: Water (plus 0.05% TFA), Mobile Phase B: ACN; Flow rate: 20 mL/min; Gradient: 35% B to 40% B in 6.5 min; Detector: UV 210 nm; Retention time: 6.54 min. The fractions containing the desired product were collected and concentrated under reduced pressure to afford the desired product. The product was separated by Prep Chiral HPLC with the following conditions: Column: CHIRALPAK IE, 2×25 cm, 5 um; Mobile Phase A: Hex (plus 0.1% FA)-HPLC, Mobile Phase B: EtOH-HPLC; Flow rate: 20 mL/min; Gradient: 15% B to 15% B in 13 min; Detector: UV: 220/254 nm; Retention Time 1: 9.62 min; Retention Time 2: 11.27 min; Injection Volume: 0.8 mL; Number Of Runs: 7; The faster-eluting isomer at 9.62 min was obtained as Compound 268 ((4aS,6R)-6-(2,3-dichloro-6-hydroxyphenyl)-3-(hydroxymethyl)-hexahydropyrrolo[1,2-c][1,3]oxazin-1-one isomer 1) as an off-white solid (23.6 mg, 22.36%): LCMS (ESI) calc'd for C₁₄H₁₅Cl₂NO₄ [M+H]⁺: 332, 334 (3:2), found 332, 334 (3:2); ¹H NMR (400 MHz, CD₃OD) δ 7.25 (d, J=8.8 Hz, 1H), 6.76 (d, J=8.9 Hz, 1H), 4.53 (d, J=6.1 Hz, 1H), 4.35-4.18 (m, 1H), 4.12-4.06 (m, 1H), 3.97-3.83 (m, 1H), 3.83-3.64 (m, 2H), 3.57-3.50 (m, 1H), 2.47-2.25 (m, 2H), 2.25-2.11 (m, 1H), 1.94-1.78 (m, 1H). The slower-eluting isomer at 11.27 min was obtained as Compound 269 ((4aS,6R)-6-(2,3-dichloro-6-hydroxyphenyl)-3-(hydroxymethyl)-hexahydropyrrolo[1,2-c][1,3]oxazin-1-one isomer 2) as an off-white solid (31.3 mg, 29.66%): LCMS (ESI) calc'd for C₁₄H₁₅Cl₂NO₄ [M+H]⁺: 332, 334 (3:2) found 332, 334 (3:2); ¹H NMR (400 MHz, CD₃OD) δ 7.25 (d, J=8.8 Hz, 1H), 6.76 (d, J=8.8 Hz, 1H), 4.44-4.36 (m, 1H), 4.34-4.22 (m, 1H), 4.12-4.07 (m, 1H), 3.93-3.81 (m, 1H), 3.73 (qd, J=12.1, 4.4 Hz, 2H), 3.55-3.50 (m, 1H), 2.45-2.39 (m, 1H), 2.34-2.27 (m, 1H), 2.25-2.15 (m, 1H), 1.71-1.56 (m, 1H).

### Example 99. Compound 270 ((6R,7aS)-6-(2,3-dichloro-6-hydroxyphenyl)-1-(2-hydroxyethyl)-tetrahydro-1H-pyrrolo[1,2-c][1,3]oxazol-3-one isomer 1) and Compound 271 ((6R,7aS)-6-(2,3-dichloro-6-hydroxyphenyl)-1-(2-hydroxyethyl)-tetrahydro-1H-pyrrolo[1,2-c][1,3]oxazol-3-one isomer 2)

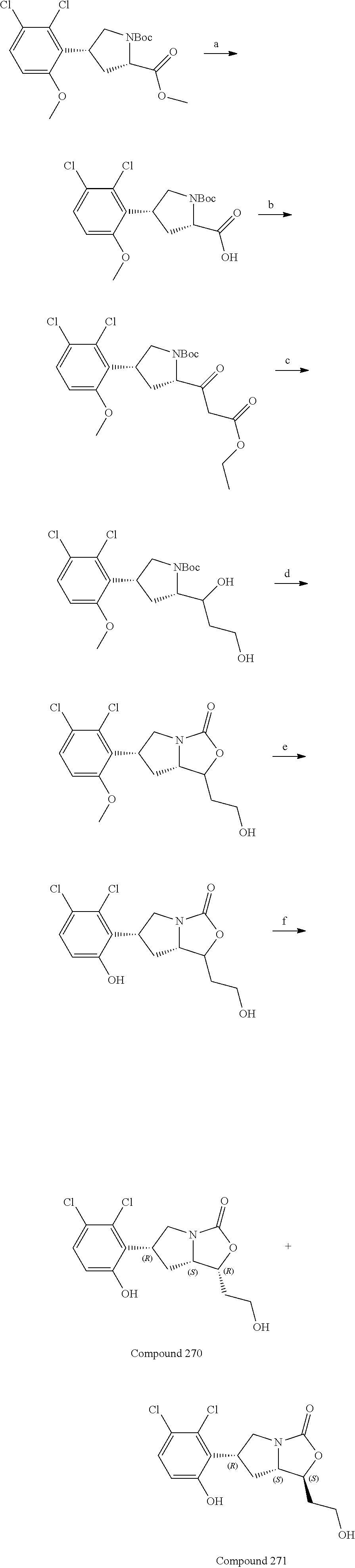

Step a:

To a stirred mixture of 1-tert-butyl 2-methyl (2R,4R)-4-(2,3-dichloro-6-methoxyphenyl)pyrrolidine-1,2-dicarboxylate (1.50 g, 3.71 mmol) in MeOH (10 mL) and H₂O (5 mL) was added LiOH H₂O (0.310 g, 7.38 mmol) at room temperature. The reaction was stirred for 1 h, acidified to pH 4 with citric acid and extracted with EA (3×30 mL). The combined organic layers were washed with brine (2×30 mL) and dried over anhydrous Na₂SO₄. After filtration, the filtrate was concentrated under reduced pressure to afford (2R,4R)-1-(tert-butoxycarbonyl)-4-(2,3-dichloro-6-methoxyphenyl)pyrrolidine-2-carboxylic acid as an off-white solid (1.20 g, 83%): LCMS (ESI) calc'd for C₁₇H₂₁Cl₂NO₅ [M+Na]⁺: 412, 414 (3:2) found 412, 414 (3:2).

Step b:

To a stirred mixture of (2R,4R)-1-(tert-butoxycarbonyl)-4-(2,3-dichloro-6-methoxyphenyl)pyrrolidine-2-carboxylic acid (1.50 g, 3.84 mmol) and Meldrum's acid (0.83 g, 5.77 mmol) in DCM (15 mL) was added DMAP (0.700 g, 5.77 mmol) and EDCI (1.11 g, 5.77 mmol) in portions at room temperature. The reaction was stirred for 1 h, washed with aq. HCl (1 M, 2×10 mL) and brine (2×20 mL) and dried over anhydrous Na₂SO₄. After filtration, the filtrate was concentrated under reduced pressure. The residue was dissolved in EtOH (10 mL), stirred at 90° C. for 1 h and evaporated. The residue was purified by reverse phase chromatography, eluting with 45% ACN in water (plus 0.05% TFA) to afford tert-butyl (2R,4R)-4-(2,3-dichloro-6-methoxyphenyl)-2-(3-ethoxy-3-oxopropanoyl)pyrrolidine-1-carboxylate as an off-white solid (0.900 g, 51%): LCMS (ESI) calc'd for C₂₁H₂₇Cl₂NO₆ [M+H]+: 460, 462 (3:2) found 460, 462 (3:2).

Step c:

To a stirred solution of tert-butyl (2R,4R)-4-(2,3-dichloro-6-methoxyphenyl)-2-(3-ethoxy-3-oxopropanoyl)pyrrolidine-1-carboxylate (0.900 g, 1.96 mmol) in MeOH (10 mL) was added NaBH₄ (0.150 g, 3.91 mmol) in portions at room temperature. The reaction was stirred for 1 h, quenched with saturated aq. NH₄Cl (20 mL) and extracted with EA (3×20 mL). The combined organic layers were washed with brine (2×20 mL) and dried over anhydrous Na₂SO₄. After filtration, the filtrate was concentrated under reduced pressure. The residue was purified by reverse phase chromatography, eluting with 40% ACN in water (plus 0.05% TFA) to afford tert-butyl (2S,4R)-4-(2,3-dichloro-6-methoxyphenyl)-2-(1,3-dihydroxypropyl)pyrrolidine-1-carboxylate as a colorless oil (600 mg, 66%): LCMS (ESI) calc'd for C₁₉H₂₇Cl₂NO₅ [M+H]⁺: 420, 422 (3:2) found 420, 422 (3:2); ¹H NMR (300 MHz, CDCl₃) δ 7.35 (d, J=8.9 Hz, 1H), 6.77 (d, J=8.9 Hz, 1H), 4.07-3.89 (m, 2H), 3.89-3.83 (m, 4H), 3.83-3.71 (m, 2H), 2.90-2.13 (m, 2H), 1.81-1.61 (m, 4H), 1.50 (d, J=4.2 Hz, 9H).

Step d:

A mixture of tert-butyl (2S,4R)-4-(2,3-dichloro-6-methoxyphenyl)-2-(1,3-dihydroxypropyl)pyrrolidine-1-carboxylate (0.300 g, 0.65 mmol) and NaH (39.0 mg, 0.97 mmol, 60% in oil) in DMF (3 mL) was stirred at 0° C. for 2 h under nitrogen atmosphere. The resulting mixture was quenched with saturated aq. NH₄Cl (20 mL) at 0° C. followed by extraction with EA (3×20 mL). The combined organic layers were washed with brine (3×20 mL) and dried over anhydrous Na₂SO₄. After filtration, the filtrate was concentrated under reduced pressure. The residue was purified by reverse phase chromatography, eluting with 40% ACN in water (plus 0.05% TFA) to afford (6R,7aS)-6-(2,3-dichloro-6-methoxyphenyl)-1-(2-hydroxyethyl)tetrahydro-1H,3H-pyrrolo[1,2-c]oxazol-3-one as a yellow oil (0.200 g, 79%): LCMS (ESI) calc'd for C₁₅H₁₇Cl₂NO₄ [M+H]⁺: 346, 348 (3:2) found 346, 348 (3:2); ¹H NMR (400 MHz, CDCl₃) δ 7.35 (d, J=8.9 Hz, 1H), 6.79 (d, J=8.9 Hz, 1H), 5.00-4.50 (m, 1H), 4.41-4.22 (m, 0.5H), 4.17-4.05 (m, 0.5H), 4.02-3.87 (m, 4H), 3.85 (s, 3H), 3.50-3.23 (m, 2H), 2.19-1.84 (m, 2H), 1.80-1.63 (m, 2H).

Step e:

A mixture of (6R,7aS)-6-(2,3-dichloro-6-methoxyphenyl)-1-(2-hydroxyethyl)tetrahydro-1H,3H-pyrrolo[1,2-c]oxazol-3-one (0.200 g, 0.58 mmol) and BBr₃ (0.20 mL) in DCM (3 mL) was stirred at room temperature for 2 h. The resulting mixture was quenched with MeOH (2 mL) and concentrated under reduced pressure. The residue was purified by Prep-HPLC with the following conditions: Column: X Select CSH Prep C18 OBD Column, 19×250 mm, 5 μm; Mobile Phase A: water (plus 0.1% FA), Mobile Phase B: ACN; Flow rate: 20 mL/min; Gradient: 30% B to 45% B in 6.5 min; Detector: UV 210 nm; Retention time: 6.54 min. The fractions containing desired product were collected and concentrated under reduced pressure to afford (6R,7aS)-6-(2,3-dichloro-6-hydroxyphenyl)-1-(2-hydroxyethyl)-tetrahydro-1H-pyrrolo[1,2-c][1,3]oxazol-3-one as an off-white solid (52.4 mg, 27%): LCMS (ESI) calc'd for C₁₄H₁₅Cl₂NO₄ [M+H]⁺: 332, 334 (3:2) found 332, 334 (3:2); ¹H NMR (300 MHz, CD₃OD) δ 7.23 (d, J=8.7 Hz, 1H), 6.73 (d, J=8.7 Hz, 1H), 4.98-4.89 (m, 0.5H), 4.68-4.60 (m, 0.5H), 4.48-4.26 (m, 1H), 4.25-4.12 (m, 1H), 4.03-3.85 (m, 1H), 3.79-3.67 (m, 2H), 3.48-3.36 (m, 1H), 2.44-2.15 (m, 1H), 2.08-1.78 (m, 3H).

Step f:

The product (6R,7aS)-6-(2,3-dichloro-6-hydroxyphenyl)-1-(2-hydroxyethyl)-tetrahydro-1H-pyrrolo[1,2-c][1,3]oxazol-3-one (40.0 mg, 0.12 mmol) was separated by Prep Chiral HPLC with the following conditions: Column: CHIRALPAK IC, 2×25 cm, 5 μm; Mobile Phase A: Hex (plus 8 mmol/L NH₃ MeOH)-HPLC, Mobile Phase B: EtOH-HPLC; Flow rate: 20 mL/min; Gradient: 20% B to 20% B in 11.5 min; Detector: UV 220/254 nm; Retention time 1: 7.81 min; Retention time 2: 9.41 min. The faster-eluting isomer at 7.81 min was obtained as Compound 270 ((6R,7aS)-6-(2,3-dichloro-6-hydroxyphenyl)-1-(2-hydroxyethyl)-tetrahydro-1H-pyrrolo[1,2-c][1,3]oxazol-3-one isomer 1) as an off-white solid (5.1 mg, 13%): LCMS (ESI) calc'd for C₁₄H₁₅Cl₂NO₄ [M+H]⁺: 332, 334 (3:2) found 332, 334 (3:2); ¹H NMR (400 MHz, CD₃OD) δ 7.23 (d, J=8.8 Hz, 1H), 6.73 (d, J=8.8 Hz, 1H), 4.96-4.89 (m, 1H), 4.41-4.28 (m, 1H), 4.23-4.12 (m, 1H), 3.97 (dd, J=10.7, 7.4 Hz, 1H), 3.79-3.66 (m, 2H), 3.43-3.39 (m, 1H), 2.39-2.33 (m, 1H), 2.06-1.80 (m, 3H). The slower-eluting isomer at 9.41 min was obtained as Compound 271 ((6R,7aS)-6-(2,3-dichloro-6-hydroxyphenyl)-1-(2-hydroxyethyl)-tetrahydro-1H-pyrrolo[1,2-c][1,3]oxazol-3-one isomer 2) as an off-white solid (9.4 mg, 23%): LCMS (ESI) calc'd for C₁₄H₁₅Cl₂NO₄ [M+H]⁺: 332, 334 (3:2) found 332, 334 (3:2); ¹H NMR (400 MHz, CD₃OD) δ 7.23 (d, J=8.6 Hz, 1H), 6.74 (d, J=8.7 Hz, 1H), 4.68-4.54 (m, 1H), 4.44-4.27 (m, 1H), 4.01-3.87 (m, 2H), 3.79-3.71 (m, 2H), 3.45-3.40 (m, 1H), 2.29-2.12 (m, 2H), 2.12-1.89 (m, 2H).

### Example 100. Compound 272 ((6R,7aS)-1-(aminomethyl)-6-(2,3-dichloro-6-hydroxyphenyl)-tetrahydro-1H-pyrrolo[1,2-c][1,3]oxazol-3-one isomer 1) and Compound 273 ((6R,7aS)-1-(aminomethyl)-6-(2,3-dichloro-6-hydroxyphenyl)-tetrahydro-1H-pyrrolo[1,2-c][1,3]oxazol-3-one isomer 2)

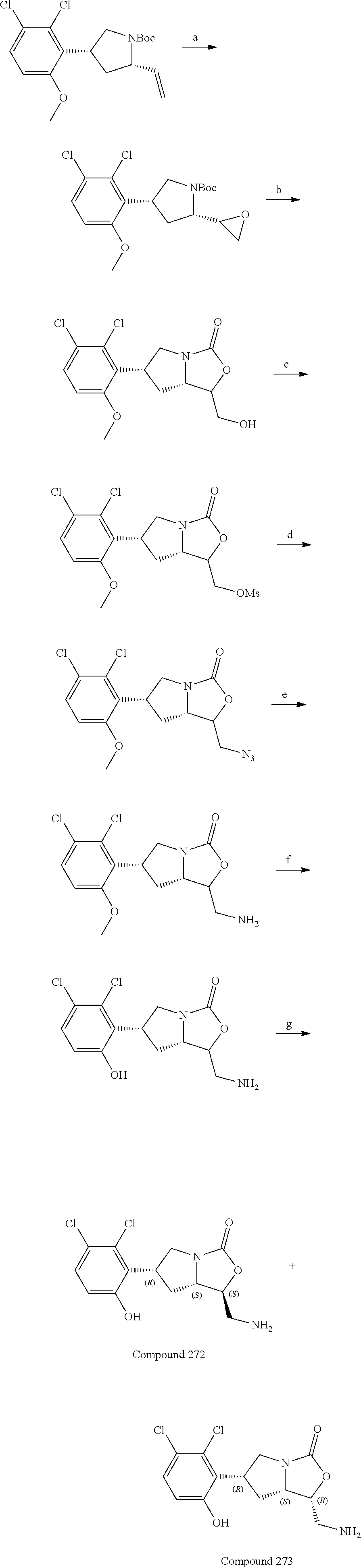

Step a:

To a stirred mixture of tert-butyl (2S,4R)-4-(2,3-dichloro-6-methoxyphenyl)-2-ethenylpyrrolidine-1-carboxylate (Example 14, step a) (3.30 g, 8.86 mmol) in DCM (25 mL) was added m-CPBA (4.59 g, 26.6 mmol) at room temperature. The reaction was stirred for 2 h, quenched with saturated aq. Na₂SO₃ (30 mL) and extracted with EA (3×30 mL). The combined organic layers were washed with saturated aq. NaHCO₃ (2×30 mL) and brine (2×30 mL) and dried over anhydrous Na₂SO₄. After filtration, the filtrate was concentrated under reduced pressure to afford tert-butyl (2S,4R)-4-(2,3-dichloro-6-methoxyphenyl)-2-(oxiran-2-yl)pyrrolidine-1-carboxylate as a light yellow oil (3.50 g, crude), which was used in the next step directly without purification: LCMS (ESI) calc'd for C₁₈H₂₃Cl₂NO₄ [M+H−56]⁺: 332, 334 (3:2) found 332, 334 (3:2).

Step b:

A stirred mixture of tert-butyl (2S,4R)-4-(2,3-dichloro-6-methoxyphenyl)-2-(oxiran-2-yl)pyrrolidine-1-carboxylate (3.30 g, 8.50 mmol) and TsOH (0.150 g, 0.85 mmol) in MeOH (25 mL) was stirred at room temperature for 3 h and concentrated under reduced pressure. The residue was purified by reverse phase chromatography, eluting with 50% ACN in water (plus 0.05% TFA) to afford (6R,7aS)-6-(2,3-dichloro-6-methoxyphenyl)-1-(hydroxymethyl)-tetrahydro-1H-pyrrolo[1,2-c][1,3]oxazol-3-one as a light yellow solid (1.7 g, 60%): LCMS (ESI) calc'd for C₁₄H₁₅Cl₂NO₄ [M+H]⁺: 332, 334 (3:2) found 332, 334 (3:2); ¹H NMR (400 MHz, CDCl₃) δ 7.35 (d, J=8.9 Hz, 1H), 6.78 (dd, J=8.9, 2.1 Hz, 1H), 4.86-4.47 (m, 1H), 4.42-4.26 (m, 1H), 4.16-3.87 (m, 3H), 3.87-3.81 (m, 4H), 3.48-3.38 (m, 1H), 2.30-2.19 (m, 1H), 2.12-1.85 (m, 1H).

Step c:

To a stirred mixture of (6R,7aS)-6-(2,3-dichloro-6-methoxyphenyl)-1-(hydroxymethyl)-tetrahydro-1H-pyrrolo[1,2-c][1,3]oxazol-3-one (0.400 g, 1.20 mmol) and TEA (0.240 g, 2.41 mmol) in DCM (5 mL) was added MsCl (0.170 g, 1.45 mmol) at 0° C. The reaction was stirred at room temperature for 1 h, diluted with saturated aq. NaHCO₃ (20 mL) and extracted with DCM (2×20 mL). The combined organic layers were washed with brine (2×20 mL) and dried over anhydrous Na₂SO₄. After filtration, the filtrate was concentrated under reduced pressure to afford [(6R,7aS)-6-(2,3-dichloro-6-methoxyphenyl)-3-oxo-tetrahydro-1H-pyrrolo[1,2-c][1,3]oxazol-1-yl]methyl methanesulfonate as a light yellow oil (0.500 g, crude), which was used in the next step directly without purification: LCMS (ESI) calc'd for C₁₅H₁₇Cl₂NO₆S [M+H]⁺: 410, 412 (3:2) found 410, 412 (3:2).

Step d:

To a stirred mixture of ((6R,7aS)-6-(2,3-dichloro-6-methoxyphenyl)-3-oxotetrahydro-1H,3H-pyrrolo[1,2-c]oxazol-1-yl)methyl methanesulfonate (0.150 g, 0.37 mmol) in DMSO (2 mL) was added NaN₃ (14.0 mg, 0.22 mmol) at room temperature under nitrogen atmosphere. The reaction was stirred at 80° C. for 2 h, cooled to room temperature, diluted with water (20 mL) and extracted with EA (3×10 mL). The combined organic layers were washed with brine (3×20 mL) and dried over anhydrous Na₂SO₄. After filtration, the filtrate was concentrated under reduced pressure to afford (6R,7aS)-1-(azidomethyl)-6-(2,3-dichloro-6-methoxyphenyl)tetrahydro-1H,3H-pyrrolo[1,2-c]oxazol-3-one, which was used in the next step directly without further purification: LCMS (ESI) calc'd for C₁₄H₁₄Cl₂N₄O₃ [M+H+MeCN]⁺: 398, 400 (3:2) found 398, 400 (3:2).

Step e:

To a stirred solution of (6R,7aS)-1-(azidomethyl)-6-(2,3-dichloro-6-methoxyphenyl)tetrahydro-1H,3H-pyrrolo[1,2-c]oxazol-3-one (0.150 g, 0.42 mmol) in EA (2 mL) was added PtO₂ (48.0 mg, 0.21 mmol) under nitrogen atmosphere. The suspension was degassed under reduced pressure and purged with hydrogen three times. The mixture was stirred under hydrogen atmosphere (1.5 atm) at room temperature for 4 h. Then the reaction was filtered and concentrated under reduced pressure. The residue was purified by reverse phase chromatography, eluting with 35% ACN in water (plus 0.05% TFA) to afford (6R,7aS)-1-(aminomethyl)-6-(2,3-dichloro-6-methoxyphenyl)tetrahydro-1H,3H-pyrrolo[1,2-c]oxazol-3-one as an off-white solid (76 mg, 55%): LCMS (ESI) calc'd for C₁₄H₁₆Cl₂N₂O₃ [M+H]⁺: 331, 333 (3:2) found 331, 333 (3:2).

Step f:

To a stirred mixture of (6R,7aS)-1-(aminomethyl)-6-(2,3-dichloro-6-methoxyphenyl)tetrahydro-1H,3H-pyrrolo[1,2-c]oxazol-3-one (90.0 mg, 0.27 mmol) in DCM (2 mL) was added BBr₃ (0.05 mL) at room temperature. The resulting mixture was stirred for 2 h, quenched with MeOH (4 mL) and concentrated under reduced pressure. The residue was purified by reverse phase chromatography, eluting with 35% ACN in water (0.05% TFA) to afford (6R,7aS)-1-(aminomethyl)-6-(2,3-dichloro-6-hydroxyphenyl)-tetrahydro-1H-pyrrolo[1,2-c][1,3]oxazol-3-one as an off-white solid (36.5 mg, 31%): LCMS (ESI) calc'd for C₁₃H₁₄Cl₂N₂O₃ [M+H]⁺: 317, 319 (3:2) found 317, 319 (3:2); ¹H NMR (400 MHz, CD₃OD) δ 7.26 (dd, J=8.8, 2.1 Hz, 1H), 6.75 (dd, J=8.8, 1.9 Hz, 1H), 4.73 (d, J=9.5 Hz, 1H), 4.47-4.26 (m, 1H), 4.03-3.88 (m, 2H), 3.51-3.35 (m, 2H), 3.31-3.21 (m, 1H), 2.35-1.84 (m, 2H).

Step g:

The product (6R,7aS)-1-(aminomethyl)-6-(2,3-dichloro-6-hydroxyphenyl)-tetrahydro-1H-pyrrolo[1,2-c][1,3]oxazol-3-one (34.0 mg, 0.08 mmol) was separated by Prep Chiral HPLC with the following conditions: Column: CHIRALPAK IG, 2×25 cm, 5 μm; Mobile Phase A: Hex (plus 8 mM NH₃ MeOH)-HPLC, Mobile Phase B: EtOH-HPLC; Flow rate: 20 mL/min; Gradient: 30% B to 30% B in 22 min; Detector: UV 220/254 nm; Retention time 1: 4.34 min; Retention time 2: 17.34 min. The faster-eluting isomer at 4.34 min was obtained as Compound 272 ((6R,7aS)-1-(aminomethyl)-6-(2,3-dichloro-6-hydroxyphenyl)-tetrahydro-1H-pyrrolo[1,2-c][1,3]oxazol-3-one isomer 1) as an off-white solid (3.4 mg, 14%): LCMS (ESI) calc'd for C₁₃H₁₄Cl₂N₂O₃ [M+H]⁺: 317, 319 (3:2) found 317, 319 (3:2); ¹H NMR (400 MHz, CD₃OD) δ 7.24 (d, J=8.8 Hz, 1H), 6.74 (d, J=8.7 Hz, 1H), 4.78-4.68 (m, 1H), 4.43-4.30 (m, 1H), 4.27-4.17 (m, 1H), 3.95 (dd, J=10.7, 7.4 Hz, 1H), 3.43-3.38 (m, 1H), 3.01 (dd, J=13.7, 8.6 Hz, 1H), 2.92 (dd, J=13.6, 4.5 Hz, 1H), 2.35-2.30 (m, 1H), 1.92-1.82 (m, 1H). The slower-eluting isomer at 17.34 min was obtained as Compound 273 ((6R,7aS)-1-(aminomethyl)-6-(2,3-dichloro-6-hydroxyphenyl)-tetrahydro-1H-pyrrolo[1,2-c][1,3]oxazol-3-one isomer 2) as an off-white solid (5.2 mg, 21%): LCMS (ESI) calc'd for C₁₃H₁₄Cl₂N₂O₃ [M+H]⁺: 317, 319 (3:2) found 317, 319 (3:2); ¹H NMR (400 MHz, CD₃OD) δ 7.24 (d, J=8.8 Hz, 1H), 6.74 (d, J=8.8 Hz, 1H), 4.52-4.45 (m, 1H), 4.45-4.33 (m, 1H), 3.99-3.87 (m, 2H), 3.45-3.40 (m, 1H), 2.97 (d, J=5.7 Hz, 2H), 2.32-2.14 (m, 2H).

### Example 101. Compound 274 (N-(((6R,7aS)-6-(2,3-dichloro-6-hydroxyphenyl)-3-oxotetrahydro-1H,3H-pyrrolo[1,2-c]oxazol-1-yl)methyl)-2-hydroxyacetamide isomer 1) and Compound 275 (N-(((6R,7aS)-6-(2,3-dichloro-6-hydroxyphenyl)-3-oxotetrahydro-1H,3H-pyrrolo[1,2-c]oxazol-1-yl)methyl)-2-hydroxyacetamide isomer 2)

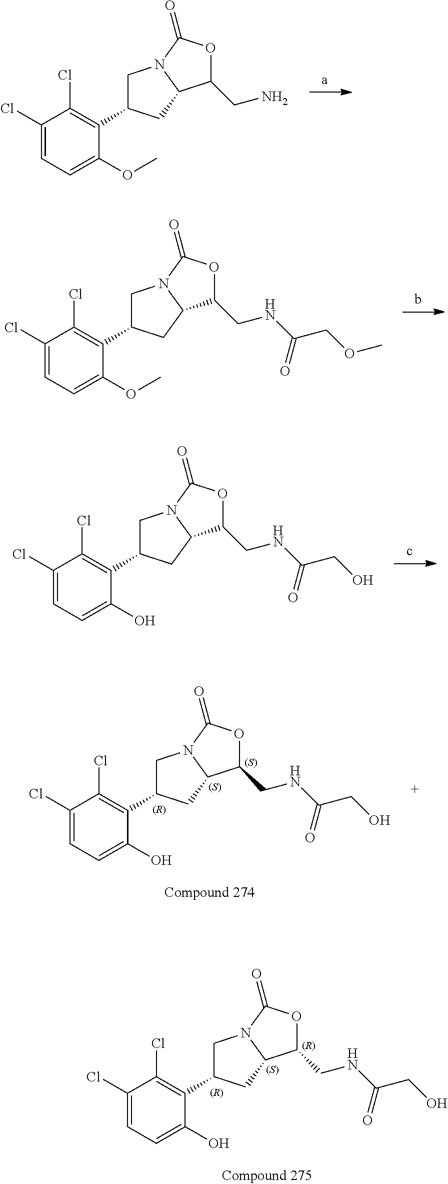

Step a:

To a stirred solution of HATU (0.240 g, 0.63 mmol) and methoxyacetic acid (46.0 mg, 0.51 mmol) in DMF (2 mL) were added (6R,7aS)-1-(aminomethyl)-6-(2,3-dichloro-6-methoxyphenyl)-tetrahydro-1H-pyrrolo[1,2-c][1,3]oxazol-3-one (0.140 g, 0.42 mmol) and TEA (86.0 mg, 0.85 mmol) at room temperature. The reaction was stirred for 2 h, quenched with water (20 mL) and extracted with EA (3×20 mL). The combined organic layers were washed with brine (5×20 mL) and dried over anhydrous Na₂SO₄. After filtration, the filtrate was concentrated under reduced pressure. The residue was purified by reverse phase chromatography, eluting with 50% ACN in water (plus 0.05% TFA) to afford N-[[(6R,7aS)-6-(2,3-dichloro-6-methoxyphenyl)-3-oxo-tetrahydro-1H-pyrrolo[1,2-c][1,3]oxazol-1-yl]methyl]-2-methoxyacetamide as a colorless oil (0.130 g, 76%): LCMS (ESI) calc'd for C₁₇H₂₀Cl₂N₂O₅ [M+H]⁺: 403, 405 (3:2) found 403, 405 (3:2); ¹H NMR (400 MHz, CDCl₃) δ 7.35 (dd, J=8.9, 3.9 Hz, 1H), 7.17-7.01 (m, 1H), 6.78 (dd, J=8.9, 4.1 Hz, 1H), 5.75-5.70 (m, 1H), 4.83-4.51 (m, 1H), 4.41-4.07 (m, 1H), 4.07-3.89 (m, 2H), 3.89-3.79 (m, 5H), 3.62-3.52 (m, 1H), 3.45 (s, 3H), 3.44-3.24 (m, 1H), 2.29-1.86 (m, 2H).

Step b:

To a stirred solution of N-[[(6R,7aS)-6-(2,3-dichloro-6-methoxyphenyl)-3-oxo-tetrahydro-1H-pyrrolo[1,2-c][1,3]oxazol-1-yl]methyl]-2-methoxyacetamide (0.130 g, 0.32 mmol) in DCM (2 mL) was added BBr₃ (0.810 g, 3.22 mmol) at room temperature. The reaction was stirred for 1 h, quenched with water (2 mL), neutralized to pH 8 with saturated aq. NaHCO₃ (20 mL) and extracted with EA (3×20 mL). The combined organic layers were washed with brine (3×20 mL) and dried over anhydrous Na₂SO₄. After filtration, the filtrate was concentrated under reduced pressure. The residue was purified by Prep-HPLC with the following conditions Column: X Select CSH Prep C18 OBD Column, 19×250 mm, 5 m; Mobile Phase A: water (plus 0.05% TFA), Mobile Phase B: ACN; Flow rate: 20 mL/min; Gradient: 40% B to 45% B in 6.5 min; Detector: UV 210 nm; Retention Time: 6.45 min. The fractions containing the desired product were collected and concentrated under reduced pressure to afford N-[[(6R,7aS)-6-(2,3-dichloro-6-hydroxyphenyl)-3-oxo-tetrahydro-1H-pyrrolo[1,2-c][1,3]oxazol-1-yl]methyl]-2-hydroxyacetamide as an off-white solid (40.0 mg, 33%): LCMS (ESI) calc'd C₁₅H₁₆Cl₂N₂O₅ for [M+H]⁺: 375, 377 (3:2) found 375, 377 (3:2): ¹H NMR (400 MHz, CD₃OD) δ 7.24 (dd, J=8.8, 3.5 Hz, 1H), 6.74 (dd, J=8.8, 4.3 Hz, 1H), 4.64-4.58 (m, 1H), 4.43-4.19 (m, 1H), 4.02 (d, J=8.3 Hz, 2H), 3.99-3.87 (m, 2H), 3.75-3.49 (m, 2H), 3.46-3.36 (m, 1H), 2.44-1.87 (m, 2H).

Step c:

The N-[[(6R,7aS)-6-(2,3-dichloro-6-hydroxyphenyl)-3-oxo-tetrahydro-1H-pyrrolo[1,2-c][1,3]oxazol-1-yl]methyl]-2-hydroxyacetamide (36.0 mg, 0.10 mmol) was separated by Prep Chiral HPLC with the following conditions: Column: CHIRALPAK IG, 2×25 cm, 5 m; Mobile Phase A: Hex (plus 0.5% 2 NH₃-MeOH)-HPLC, Mobile Phase B: EtOH-HPLC; Flow rate: 20 mL/min; Gradient: 30% B to 30% B in 32 min; Detector UV 254/220 nm; Retention Time 1:19.66 mi; Retention Time 2: 26.24 mi; Injection Volume: 0.5 mL; Number Of Runs: 3. The faster-eluting isomer at 19.66 min was obtained as Compound 274 (N-[[(6R,7aS)-6-(2,3-dichloro-6-hydroxyphenyl)-3-oxo-tetrahydro-1H-pyrrolo[1,2-c][1,3]oxazol-1-yl]methyl]-2-hydroxyacetamide isomer 1) as an off-white solid (4.30 mg, 11.9% o): LCMS (ESI) calc'd C₁₅H₁₆Cl₂N₂O₅ for [M+H]⁺: 375, 377 (3:2) found 375, 377 (3:2); ¹H NMR (400 Hz, CD₃OD) δ 7.24 (d, J=8.8 Hz, 1H), 6.75 (d, J=8.8 Hz, 1H), 4.93-4.90 (m, 1H), 4.44-4.29 (m, 1H), 4.27-4.17 (m, 1H), 4.01 (s, 2H), 3.96 (dd, J=10.7, 7.4 Hz, 1H), 3.70 (dd, J=14.1, 4.6 Hz, 1H), 3.54 (dd, J=14.1, 8.4 Hz, 1H), 3.45-3.39 (m, 1H), 2.40-2.36 (m, 1H), 1.98-1.86 (m, 1H). The slower-eluting isomer at 19.66 min was obtained as Compound 275 (N-[[(6R,7aS)-6-(2,3-dichloro-6-hydroxyphenyl)-3-oxo-tetrahydro-1H-pyrrolo[1,2-c][1,3]oxazol-1-yl]methyl]-2-hydroxyacetamide isomer 2) as an off-white solid (12.0 mg, 33.30%): LCMS (ESI) calc'd C₁₅H₁₆Cl₂N₂O₅ for [M+H]⁺: 375, 377 (3:2) found 375, 377 (3:2): ¹H NM/R (400 MHz, CD₃OD) δ 7.23 (d, J=8.8 Hz, 1H), 6.73 (d, J=8.8 Hz, 1H), 4.66-4.56 (m, 1H), 4.46-4.28 (m, 1H), 4.03 (s, 2H), 4.00-3.85 (m, 2H), 3.67-3.55 (m, 2H), 3.45-3.39 (m, 1H), 2.31-2.09 (m, 2H).

Example 102. Compounds 276-279 were prepared in an analogous fashion as that described for Compounds 274-275.

| Compound | | | |
|---|---|---|---|
| Number | Structure | Chemical Name | MS: (M + H)⁺ & ¹H MNR |
| 276 | 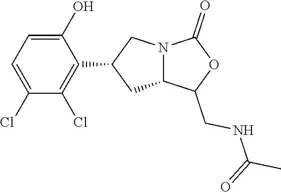 [SMILES: CC(=O)NCC1OC(=O)N2C[C@@H](c3c(O)ccc(Cl)c3Cl)C[C@@H]12] | N-[[(6R,7aS)-6-(2,3- dichloro-6- hydroxyphenyl)-3- oxo-tetrahydro-1H- pyrrolo[1,2- c][1,3]oxazol-1- yl]methyl]acetamide | [M + H]⁺: 359, 361 (3:2); ¹H NMR (400 MHz, CD₃OD) δ 7.24 (dd, J = 8.8, 3.1 Hz, 1H), 6.74 (dd, J = 8.8, 3.2 Hz, 1H), 4.59-4.54 (m, 1H), 4.43-4.33 (m, 1H), 3.98-3.89 (m, 2H), 3.66-3.50 (m, 2H), 3.45-3.38 (m, 1H), 2.41-2.31 (m, 1H), 2.27-2.16 (m, 1H), 1.99 (d, J = 8.4 Hz, 3H). |
| 277 | 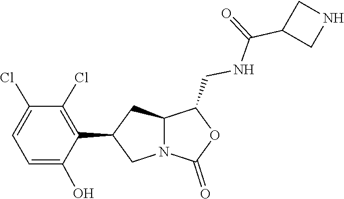 [SMILES: O=C(NC[C@@H]1OC(=O)N2C[C@@H](c3c(O)ccc(Cl)c3Cl)C[C@@H]12)C1CNC1] | N-[[(6R,7aS)-6-(2,3- dichloro-6- hydroxyphenyl)-3- oxo-tetrahydro-1H- pyrrolo[1,2- c][1,3]oxazol-1- yl]methyl]azetidine-3- carboxamide isomer 1 | [M + H]⁺: 400, 402 (3:2); ¹H NMR (400 MHz, CD₃OD) δ 7.24 (d, J = 8.8 Hz, 1H), 6.74 (d, J = 8.8 Hz, 1H), 4.62-4.55 (m, 1H), 4.42-4.33 (m, 1H), 4.02-3.87 (m, 3H), 3.85-3.78 (m, 1H), 3.66-3.28 (m, 6H), 2.28-2.15 (m, 2H). |
| 278 | 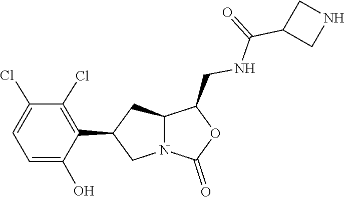 [SMILES: O=C(NC[C@H]1OC(=O)N2C[C@@H](c3c(O)ccc(Cl)c3Cl)C[C@@H]12)C1CNC1] | N-[[(6R,7aS)-6-(2,3- dichloro-6- hydroxyphenyl)-3- oxo-tetrahydro-1H- pyrrolo[1,2- c][1,3]oxazol-1- yl]methyl]azetidine-3- carboxamide isomer 2 | [M + H]⁺: 400, 402 (3:2); ¹H NMR (400 MHz, CD₃OD) δ 7.25 (d, J = 8.8 Hz, 1H), 6.74 (d, J = 8.8 Hz, 1H), 4.89-4.81 (m, 1H), 4.43-4.32 (m, 1H), 4.27-4.19 (m, 1H), 4.03-3.92 (m, 2H), 3.87-3.78 (m, 1H), 3.71-3.25 (m, 6H), 2.41-2.32 (m, 1H), 1.95-1.86 (m, 1H). |

### Example 103. Compound 279 ((6R,7aS)-6-(2,3-dichloro-6-hydroxyphenyl)-1-(piperazin-1-ylmethyl)-tetrahydro-1H-pyrrolo[1,2-c][1,3]oxazol-3-one isomer 1) and Compound 280 ((6R,7aS)-6-(2,3-dichloro-6-hydroxyphenyl)-1-(piperazin-1-ylmethyl)-tetrahydro-1H-pyrrolo[1,2-c][1,3]oxazol-3-one isomer 2)

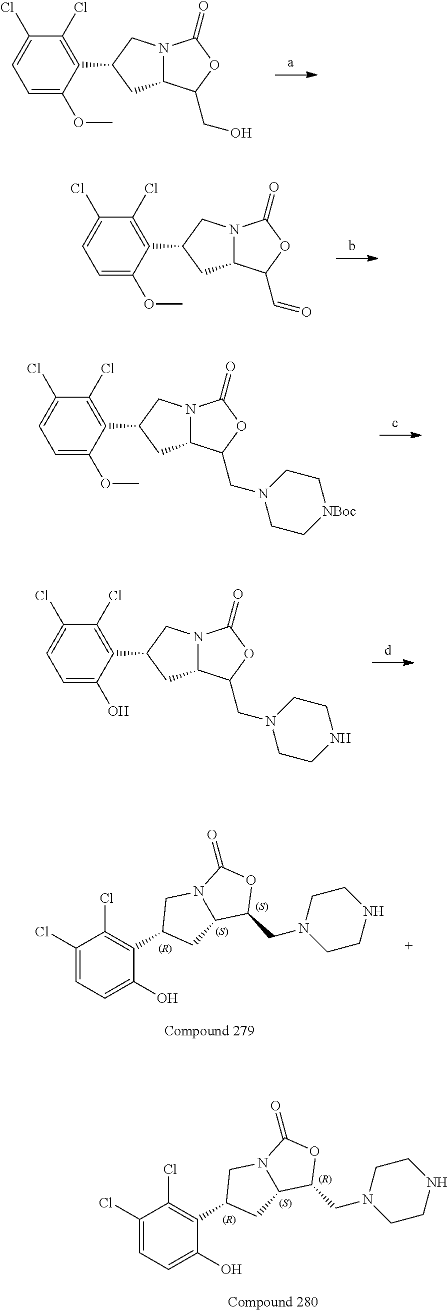

Step a:

To a stirred solution of (6R,7aS)-6-(2,3-dichloro-6-methoxyphenyl)-1-(hydroxymethyl)-tetrahydro-1H-pyrrolo[1,2-c][1,3]oxazol-3-one (Example 18, step b) (0.400 g, 1.20 mmol) in DCM (2 mL) was added Dess-Martin periodinane (1.02 g, 2.41 mmol) at room temperature. The reaction was stirred for 3 h and quenched with saturated aq. Na₂SO₃ (20 mL) and NaHCO₃ (20 mL) at 0° C. followed by extraction with EA (3×30 mL). The combined organic layers were washed with brine (3×30 mL) and dried over anhydrous Na₂SO₄. After filtration, the filtrate was concentrated under reduced pressure. The residue was purified by reverse phase chromatography, eluting with 45% ACN in water (plus 0.1% FA) to afford (6R,7aS)-6-(2,3-dichloro-6-methoxyphenyl)-3-oxo-tetrahydro-1H-pyrrolo[1,2-c][1,3]oxazole-1-carbaldehyde as an off-white solid (0.240 g, 60%): LCMS (ESI) calc'd for C₁₄H₁₃Cl₂NO₄ [M+H]⁺ 330, 332 (3:2) found 330, 332 (3:2).

Step b:

To a stirred solution of (6R,7aS)-6-(2,3-dichloro-6-methoxyphenyl)-3-oxo-tetrahydro-1H-pyrrolo[1,2-c][1,3]oxazole-1-carbaldehyde (80.0 mg, 0.24 mmol) and tert-butyl piperazine-1-carboxylate (90.0 mg, 0.49 mmol) in DCM (3 mL) were added AcOH (15.0 mg, 0.24 mmol) and NaBH(AcO)₃ (0.150 g, 0.73 mmol) at room temperature. The reaction was stirred for 16 h and diluted with water (30 mL) followed by extraction with EA (3×30 mL). The combined organic layers were washed with brine (2×30 mL) and dried over anhydrous Na₂SO₄. After filtration, the filtrate was concentrated under reduced pressure. The residue was purified by reverse phase chromatography, eluting with 55% ACN in water (plus 0.05% TFA) to afford tert-butyl 4-[[(6R,7aS)-6-(2,3-dichloro-6-methoxyphenyl)-3-oxo-tetrahydro-1H-pyrrolo[1,2-c][1,3]oxazol-1-yl]methyl]piperazine-1-carboxylate as an off-white solid (0.100 g, 82%): LCMS (ESI) calc'd for C₂₃H₃₁Cl₂N₃O₅ [M+H]⁺ 500, 502 (3:2) found 500, 502 (3:2); ¹H NMR (400 MHz, CDCl₃) δ 7.35 (dd, J=8.9, 4.1 Hz, 1H), 6.78 (dd, J=9.4, 4.1 Hz, 1H), 5.43-4.86 (m, 2H), 4.42-4.24 (m, 1H), 4.14-4.02 (m, 1H), 4.00-3.88 (m, 1H), 3.85 (d, J=4.1 Hz, 3H), 3.60-3.32 (m, 5H), 2.91-2.44 (m, 5H), 2.30-1.84 (m, 2H), 1.49 (s, 9H).

Step c:

To a stirred solution of tert-butyl 4-[[(6R,7aS)-6-(2,3-dichloro-6-methoxyphenyl)-3-oxo-tetrahydro-1H-pyrrolo[1,2-c][1,3]oxazol-1-yl]methyl]piperazine-1-carboxylate (0.100 g, 0.20 mmol) in DCM (3 mL) was added BBr₃ (0.500 g, 2.00 mmol) at room temperature. The reaction was stirred for 3 h, quenched with MeOH (2 ml) and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 20% B to 40% B in 6.5 min; Detector: UV 254/210 nm; Retention Time: 6.45 min. The fractions containing the desired product were collected and concentrated under reduced pressure to afford (6R,7aS)-6-(2,3-dichloro-6-hydroxyphenyl)-1-(piperazin-1-ylmethyl)-tetrahydro-1H-pyrrolo[1,2-c][1,3]oxazol-3-one as an off-white solid (51.0 mg, 51%): LCMS (ESI) calc'd for C₁₇H₂₁Cl₂N₃O₃ [M+H]⁺ 386, 388 (3:2) found 386, 388 (3:2); ¹H NMR (400 MHz, CD₃OD) δ 7.25 (d, J=8.7 Hz, 1H), 6.74 (dd, J=8.8, 1.4 Hz, 1H), 5.02-4.94 (m, 0.5H), 4.73-4.68 (m, 0.5H), 4.47-4.17 (m, 1H), 4.01-3.88 (m, 2H), 3.43 (td, J=10.4, 4.6 Hz, 1H), 3.31-3.24 (m, 4H), 3.01-2.79 (m, 6H), 2.421.81 (m, 2H).

Step d:

The (6R,7aS)-6-(2,3-dichloro-6-hydroxyphenyl)-1-(piperazin-1-ylmethyl)-tetrahydro-1H-pyrrolo[1,2-c][1,3]oxazol-3-one (51.0 mg, 0.10 mmol) was separated by Prep Chiral HPLC with the following conditions: Column: CHIRALPAK IG, 20×250 mm, 5 μm; Mobile Phase A: Hex (plus 0.5% 2 M NH₃-MeOH)-HPLC, Mobile Phase B: EtOH-HPLC; Flow rate: 20 mL/min; Gradient: 30% B to 30% B in 17 min; Detector: UV 254/220 nm; Retention time 1:11.13 min; Retention time 2: 15.48 min. The faster-eluting isomer at 11.13 min was obtained as Compound 279 ((6R,7aS)-6-(2,3-dichloro-6-hydroxyphenyl)-1-(piperazin-1-ylmethyl)-tetrahydro-1H-pyrrolo[1,2-c][1,3]oxazol-3-one isomer 1) as an off-white solid (8.6 mg, 27.6%): LCMS (ESI) calc'd for C₁₇H₂Cl₂N₃O₃ [M+H]⁺: 386, 388 (3:2) found 386, 388 (3:2); ¹HNMR (400 MHz, CD₃OD) δ 7.24 (d, J=8.8 Hz, 1H), 6.74 (d, J=8.8 Hz, 1H), 5.00-4.93 (m, 1H), 4.39-4.29 (m, 1H), 4.27-4.14 (m, 1H), 3.96 (dd, J=10.7, 7.4 Hz, 1H), 3.45-3.40 (m, 1H), 2.93-2.89 (m, 4H), 2.80-2.69 (m, 2H), 2.69-2.59 (min, 2H), 2.59-2.51 (m, 2H), 2.36-2.30 (m, 1H), 1.92-1.84 (m, 1H). The slower-eluting isomer at 11.13 min was obtained as Compound 280 ((6R,7aS)-6-(2,3-dichloro-6-hydroxyphenyl)-1-(piperazin-1-ylmethyl)-tetrahydro-1H-pyrrolo[1,2-c][1,3]oxazol-3-one isomer 2) as an off-white solid (5.7 mg, 18.27% o): LCMS (ESI) calc'd for C₁₇H₂₁Cl₂N₃O₃ [M+H]⁺: 386, 388 (3:2) found 386, 388 (3:2); ¹H NM/R (400 MHz, CD3D) δ 7.24 (d, J=8.8 Hz, 1H), 6.74 (d, J=8.8 Hz, 1H), 4.71-4.63 (m, 1H), 4.46-4.30 (m, 1H), 3.97-3.87 (m, 2H), 3.46-3.40 (m, 1H), 2.95-2.90 (m, 4H), 2.82-2.67 (m, 2H), 2.67-2.54 (m, 4H), 2.29-2.16 (m, 2H).

Example 104. Compound 281-285 were prepared in an analogous fashion as that described for Compounds 279-280.

| Compound | | | |
|---|---|---|---|
| Number | Structure | Chemical Name | MS: (M + H)⁺ & ¹H MNR |
| 281 | 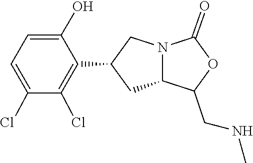 [SMILES: CNCC1OC(=O)N2C[C@@H](c3c(O)ccc(Cl)c3Cl)C[C@@H]12] | (6R,7aS)-6-(2,3- dichloro-6- hydroxyphenyl)-1- [(methylamino) methyl]-tetrahydro- 1H-pyrrolo[1,2- c][1,3]oxazol-3- one | [M + H]⁺: 331, 333 (3:2); ¹H NMR (400 MHz, CD₃OD) δ 7.26 (dd, J = 8.8, 2.3 Hz, 1H), 6.76 (d, J = 8.8, 2.2 Hz, 1H), 5.05-4.79 (m, 1H), 4.47-4.36 (m, 1H), 4.35-4.27 (m, 0.5H), 4.04-3.90 (m, 1.5H), 3.52-3.39 (m, 3H), 2.81 (d, J = 3.3 Hz, 3H), 2.35-2.23 (m, 1.5H), 1.97-1.86 (m, 0.5H). |
| 282 | 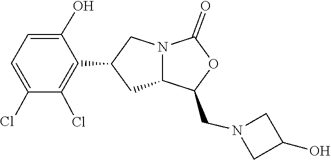 [SMILES: O=C1O[C@@H](CN2CC(O)C2)[C@@H]2C[C@H](c3c(O)ccc(Cl)c3Cl)CN12] | (6R,7aS)-6-(2,3- dichloro-6- hydroxyphenyl)-1- [(3- hydroxyazetidin-1- yl)methyl]- tetrahydro-1H- pyrrolo[1,2- c][1,3]oxazol-3- one isomer 1 | [M + H]⁺: 373, 375 (3:2); ¹H NMR (400 MHz, CD₃OD) δ 7.24 (d, J = 8.8 Hz, 1H), 6.74 (d, J = 8.8 Hz, 1H), 4.54-4.45 (m, 1H), 4.44-4.32 (m, 2H), 3.97-3.85 (m, 2H), 3.83-3.70 (m, 2H), 3.46-3.37 (m, 1H), 3.12-3.03 (m, 2H), 2.92- 2.80 (m, 2H), 2.26-2.15 (m, 2H). |
| 283 | 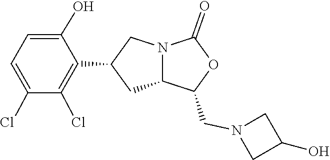 [SMILES: O=C1O[C@H](CN2CC(O)C2)[C@@H]2C[C@H](c3c(O)ccc(Cl)c3Cl)CN12] | (6R,7aS)-6-(2,3- dichloro-6- hydroxyphenyl)-1- [(3- hydroxyazetidin-1- yl)methyl]- tetrahydro-1H- pyrrolo[1,2- c][1,3]oxazol-3- one isomer 2 | [M + H]⁺: 373, 375 (3:2); ¹H NMR (400 MHz, CD₃OD) δ 7.24 (d, J = 8.8 Hz, 1H), 6.74 (d, J = 8.8 Hz, 1H), 4.78-4.70 (m, 1H), 4.41-4.29 (m, 2H), 4.22-4.14 (m, 1H), 3.94 (dd, J = 10.7, 7.3 Hz, 1H), 3.81-3.70 (m, 1H), 3.44-3.34 (m, 1H), 3.10-3.00 (m, 3H), 2.92- 2.78 (m, 2H), 2.37-2.27 (m, 1H), 1.92-1.77 (m, 1H). |
| 284 | 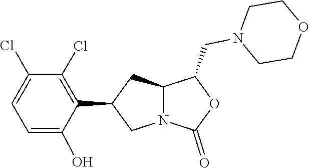 [SMILES: O=C1O[C@@H](CN2CCOCC2)[C@@H]2C[C@H](c3c(O)ccc(Cl)c3Cl)CN12] | (7S,8aR)-2-(2,3- dichloro-6- hydroxyphenyl)-7- [(3- hydroxyazetidin-1- yl)methyl]- hexahydro-1H- indolizin-5-one isomer 1 | [M + H]⁺: 387, 389 (3:2); ¹H NMR (400 MHz, CD₃OD) δ 7.24 (d, J = 8.8 Hz, 1H), 6.74 (d, J = 8.8 Hz, 1H), 4.72-4.66 (m, 1H), 4.45-4.35 (m, 1H), 3.98-3.89 (m, 2H), 3.73 (t, J = 4.7 Hz, 4H), 3.46- 3.40 (m, 1H), 2.85-2.74 (m, 2H), 2.70-2.59 (m, 4H), 2.30-2.17 (m, 2H). |
| 285 | 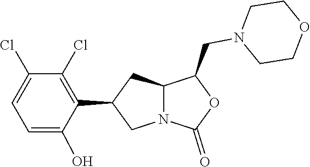 [SMILES: O=C1O[C@H](CN2CCOCC2)[C@@H]2C[C@H](c3c(O)ccc(Cl)c3Cl)CN12] | (7R,8aR)-2-(2,3- dichloro-6- hydroxyphenyl)-7- [(3- hydroxyazetidin-1- yl)methyl]- hexahydro-1H- indolizin-5-one isomer 2 | [M + H]⁺: 387, 389 (3:2); ¹H NMR (400 MHz, CD₃OD) δ 7.24 (d, J = 8.8 Hz, 1H), 6.74 (d, J = 8.8 Hz, 1H), 5.00-4.94 (m, 1H), 4.40-4.30 (m, 1H), 4.25-4.18 (m, 1H), 3.96 (dd, J = 10.7, 7.4 Hz, 1H), 3.71 (t, J = 4.7 Hz, 4H), 3.36- 3.35 (m, 1H), 2.82-2.68 (m, 2H), 2.66-2.58 (m, 2H), 2.57-2.50 (m, 2H), 2.40-2.31 (m, 1H), 1.92-1.85 |
| | | | (m, 1H). |

### Example 105. Compound 286 ((6R,7aS)-6-(2,3-dichloro-6-hydroxyphenyl)-3-oxotetrahydro-1H,3H-pyrrolo[1,2-c]oxazole-1-carboxamide isomer 1) and Compound 287 ((6R,7aS)-6-(2,3-dichloro-6-hydroxyphenyl)-3-oxotetrahydro-1H,3H-pyrrolo[1,2-c]oxazole-1-carboxamide isomer 2)

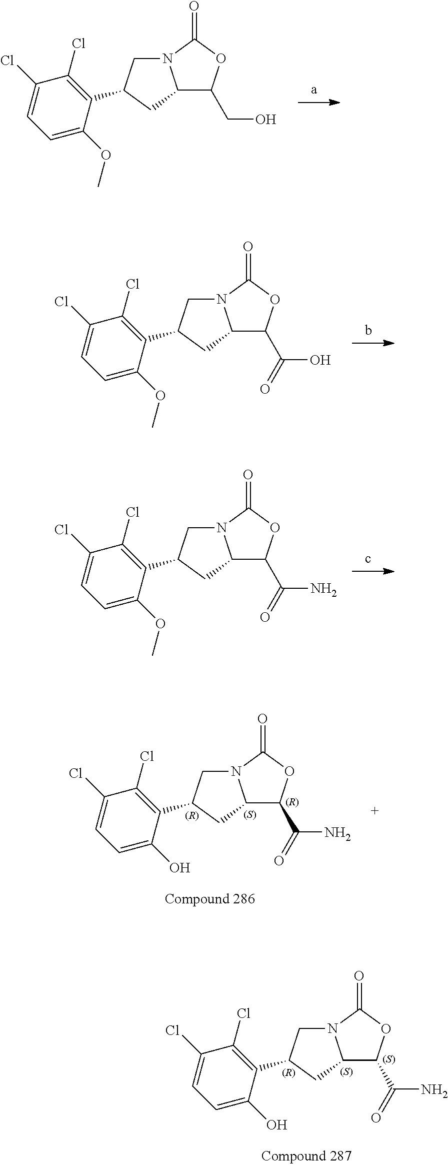

Step a.

To a stirred solution of (6R,7aS)-6-(2,3-dichloro-6-methoxyphenyl)-1-(hydroxymethyl)-tetrahydro-1H-pyrrolo[1,2-c][1,3]oxazol-3-one (Example 18, step b) (0.400 g, 1.20 mmol) in CCl₄ (3 mL) and ACN (3 mL) was added NaIO₄ (0.900 g, 4.22 mmol) in water (1 mL) at 0° C. followed by RuCl₃.H₂O (14.0 mg, 0.06 mmol). The reaction was stirred at room temperature for 16 h and diluted with water (50 mL) at 0° C. followed by extraction with EA (3×50 mL). The combined organic layers were washed with brine (3×50 mL) and dried over anhydrous Na₂SO₄. After filtration, the filtrate was concentrated under reduced pressure. The residue was purified by reverse phase chromatography, eluting with 45% ACN in water (plus 0.05% TFA) to afford (6R,7aS)-6-(2,3-dichloro-6-methoxyphenyl)-3-oxo-tetrahydro-1H-pyrrolo[1,2-c][1,3]oxazole-1- carboxylic acid as an off-white foam (0.340 g, 82%): LCMS (ESI) calc'd for C₁₄H₁₃Cl₂NO₅ [M+H]⁺: 346, 348 (3:2) found 346, 348 (3:2); ¹H NMR (400 MHz, CDCl₃) δ 7.40-7.31 (m, 1H), 6.83-6.73 (m, 1H), 5.29-4.75 (m, 1H), 4.49-4.32 (m, 1H), 4.25-4.09 (m, 1H), 4.02-3.90 (m, 1H), 3.86 (s, 3H), 3.54-3.39 (m, 1H), 2.45-2.27 (m, 1H), 2.20-2.06 (m, 1H).

Step b:

To a stirred solution of (6R,7aS)-6-(2,3-dichloro-6-methoxyphenyl)-3-oxo-tetrahydro-1H-pyrrolo[1,2-c][1,3] oxazole-1-carboxylic acid (70.0 mg, 0.20 mmol), HOBT (42.0 mg, 0.30 mmol) and EDCI (58.0 mg, 0.30 mmol) in DMF (1 mL) were added NH₄Cl (55.0 mg, 1.01 mmol) and TEA (61.0 mg, 0.61 mmol) at room temperature. The reaction was stirred at 40° C. for 24 h and diluted with water (30 mL) followed by extraction with EA (3×30 mL). The combined organic layers were washed with brine (3×30 mL) and dried over anhydrous Na₂SO₄. After filtration, the filtrate was concentrated under reduced pressure. The residue was purified by reverse phase chromatography, eluting with 50% ACN in water (plus 0.05% TFA) to afford (6R,7aS)-6-(2,3-dichloro-6-methoxyphenyl)-3-oxo-tetrahydro-1H-pyrrolo[1,2-c][1,3]oxazole-1-carboxamide as a colorless oil (40.0 mg, 57%): LCMS (ESI) calc'd for C₁₄H₁₄Cl₂N₂O₄ [M+H]⁺: 345, 347 (3:2) found 345, 347 (3:2); ¹H NMR (400 MHz, CDCl₃) δ 7.36 (d, J=8.9 Hz, 1H), 6.79 (d, J=8.9 Hz, 1H), 6.73 (s, 1H), 6.02 (s, 1H), 4.76 (d, J=2.6 Hz, 1H), 4.45-4.34 (m, 1H), 4.32-4.20 (m, 1H), 3.91 (dd, J=11.3, 6.3 Hz, 1H), 3.86 (s, 3H), 3.49-3.40 (m, 1H), 2.45-2.32 (m, 1H), 2.12-2.06 (m, 1H).

Step c:

To a stirred solution of (6R,7aS)-6-(2,3-dichloro-6-methoxyphenyl)-3-oxo-tetrahydro-1H-pyrrolo[1,2-c][1,3] oxazole-1-carboxamide (40.0 mg, 0.12 mmol) in DCM (1 mL) was added BBr₃ (0.290 g, 1.16 mmol,) at room temperature. The resulting mixture was stirred for 4 h, quenched with MeOH (2 mL) and concentrated under reduced pressure. The residue was purified by Prep-HPLC with the following conditions: Column: X Select CSH Prep C18 OBD Column, 19×250 mm, 5 μm; Mobile Phase A: Water (plus 0.05% TFA), Mobile Phase B: ACN; Flow rate: 20 mL/min; Gradient: 35% to 40% in 6.5 min; Detector: UV 254/220 nm; Retention time 1: 6.54 min; Retention time 2: 7.01 min. The faster-eluting isomer at 6.54 min was obtained as Compound 286 ((6R,7aS)-6-(2,3-dichloro-6-hydroxyphenyl)-3-oxo-tetrahydro-1H-pyrrolo[1,2-c][1,3]oxazole-1-carboxamide isomer 1) as an off-white solid (6.9 mg, 18%): LCMS (ESI) calc'd for C₁₃H₁₂Cl₂N₂O₄ [M+H]⁺: 331, 333 (3:2) found 331, 333 (3:2); ¹H NMR (400 MHz, CD₃OD) δ 7.24 (d, J=8.8 Hz, 1H), 6.74 (d, J=8.8 Hz, 1H), 5.20 (d, J=8.7 Hz, 1H), 4.49-4.33 (m, 2H), 3.98 (dd, J=10.7, 7.3 Hz, 1H), 3.49-3.43 (m, 1H), 2.25-2.19 (m, 1H), 2.03-1.93 (m, 1H). The slower-eluting isomer at 7.01 min was obtained as Compound 287 ((6R,7aS)-6-(2,3-dichloro-6-hydroxyphenyl)-3-oxo-tetrahydro-1H-pyrrolo[1,2-c][1,3]oxazole-1-carboxamide isomer 2) as an off-white solid (6.8 mg, 17.72%): LCMS (ESI) calc'd for C₁₃H₁₂Cl₂N₂O₄ [M+H]⁺: 331, 333 (3:2) found 331, 333 (3:2); ¹H NMR (400 MHz, CD₃OD) δ 7.25 (d, J=8.7 Hz, 1H), 6.75 (d, J=8.7 Hz, 1H), 4.87 (d, J=3.4 Hz, 1H), 4.48-4.35 (m, 1H), 4.20 (td, J=8.3, 3.4 Hz, 1H), 3.93 (dd, J=10.8, 6.8 Hz, 1H), 3.49-3.40 (m, 1H), 2.35-2.29 (m, 2H).

Example 106. Compounds 288-289 were prepared in an analogous fashion as that described as Compounds 286-287.

| Compound | | | |
|---|---|---|---|
| Number | Structure | Chemical Name | MS: (M + H)⁺ & ¹H MNR |
| 288 | 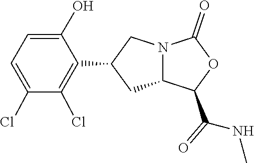 [SMILES: CNC(=O)[C@@H]1OC(=O)N2C[C@@H](c3c(O)ccc(Cl)c3Cl)C[C@@H]12] | (6R,7aS)-6-(2,3- dichloro-6- hydroxyphenyl)-N- methyl-3-oxo- tetrahydro-1H- pyrrolo[1,2- c][1,3]oxazole-1- carboxamide isomer 1 | [M + H]⁺: 345, 347 (3:2); ¹H NMR (400 MHz, CD₃OD) δ 7.24 (d, J = 8.8 Hz, 1H), 6.74 (d, J = 8.8 Hz, 1H), 4.89-4.85 (m, 1H), 4.51-4.33 (m, 1H), 4.18 (td, J = 8.3, 3.5 Hz, 1H), 3.92 (dd, J = 10.8, 6.8 Hz, 1H), 3.48-3.42 (m, 1H), 2.83 (s, 3H), 2.35-2.25 (m, 2H). |
| 289 | 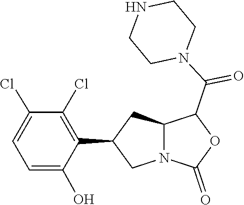 [SMILES: O=C(C1OC(=O)N2C[C@@H](c3c(O)ccc(Cl)c3Cl)C[C@@H]12)N1CCNCC1] | (6R,7aS)-6-(2,3- dichloro-6- hydroxyphenyl)-1- (piperazine-1- carbonyl)-tetrahydro- 1H-pyrrolo[1,2- c][1,3]oxazol-3-one | [M + H]⁺: 400, 402 (3:2); ¹H NMR (400 MHz, CD₃OD) δ 7.25 (d, J = 8.8 Hz, 1H), 6.75 (d, J = 8.8 Hz, 1H), 5.43 (d, J = 3.1 Hz, 1H), 4.47-4.33 (m, 2H), 3.94 (dd, J = 10.8, 6.9 Hz, 1H), 3.79-3.71 (m, 1H), 3.67-3.59 (m, 1H), 3.57- 3.43 (m, 3H), 2.95-2.81 (m, 4H), 2.41-2.23 (m, 2H). |

### Example 107. Compound 290 (6R,7aS)-6-(2,3-dichloro-6-hydroxyphenyl)-2-(2-hydroxyethyl)hexahydro-3H-pyrrolo[1,2-c]imidazol-3-one

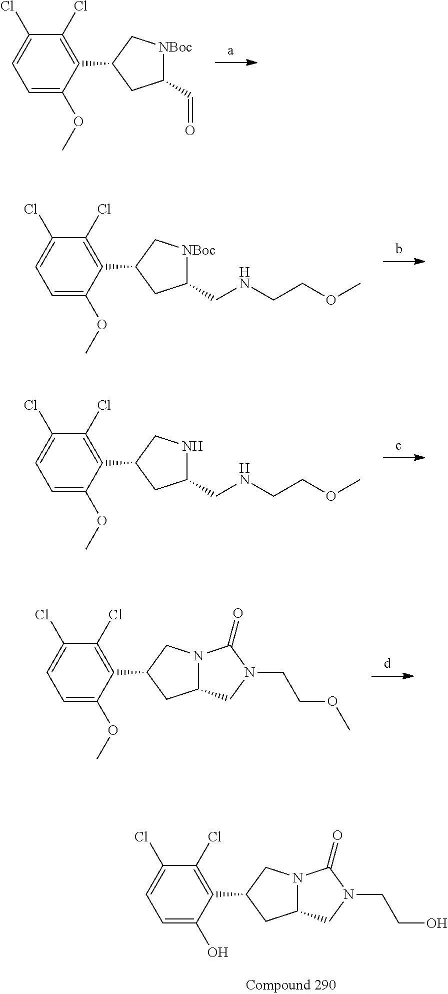 [SMILES: COc1ccc(Cl)c(Cl)c1[C@H]1C[C@@H](C=O)N(C(=O)OC(C)(C)C)C1 ; COCCNC[C@@H]1C[C@H](c2c(OC)ccc(Cl)c2Cl)CN1C(=O)OC(C)(C)C ; COCCNC[C@@H]1C[C@H](c2c(OC)ccc(Cl)c2Cl)CN1 ; COCCN1C[C@@H]2C[C@H](c3c(OC)ccc(Cl)c3Cl)CN2C1=O ; O=C1N(CCO)C[C@@H]2C[C@H](c3c(O)ccc(Cl)c3Cl)CN12]

Step a:

To a stirred solution of tert-butyl (2S,4R)-4-(2,3-dichloro-6-methoxyphenyl)-2-formylpyrrolidine-1-carboxylate (Example 7, step c) (0.500 g, 1.34 mmol) and 2-methoxyethan-1-amine (0.200 g, 2.67 mmol) in DCM (1 mL) was added NaBH(OAc)₃ (0.570 g, 2.67 mmol) at room temperature. The reaction was stirred for 2 h and quenched with saturated aq. NH₄Cl (20 mL) followed by extraction with EA (3×30 mL). The combined organic layers were washed with brine (3×20 mL) and dried over anhydrous Na₂SO₄. After filtration, the filtrate was concentrated under reduced pressure. The residue was purified by reverse phase chromatography, eluting with 50% ACN in water (plus 0.05% TFA) to afford tert-butyl (2S,4R)-4-(2,3-dichloro-6-methoxyphenyl)-2-[[(2-methoxyethyl)amino]methyl]pyrrolidine-1-carboxylate as a light yellow oil (0.500 g, 86%). LCMS (ESI) calc'd C₂₀H₃₀Cl₂N₂O₄ for [M+H]⁺: 433, 435 (3:2) found 433, 435 (3:2); ¹H NMR (400 MHz, CDCl₃) δ 7.37 (d, J=8.9 Hz, 1H), 6.78 (d, J=9.0 Hz, 1H), 4.25-4.07 (m, 2H), 3.86 (s, 3H), 3.81-3.73 (m, 1H), 3.74-3.61 (m, 3H), 3.55 (s, 1H), 3.52-3.34 (m, 4H), 3.24-3.11 (m, 2H), 2.48-2.26 (m, 2H), 1.49 (s, 9H).

Step b:

To a stirred solution of tert-butyl (2S,4R)-4-(2,3-dichloro-6-methoxyphenyl)-2-[[(2-methoxyethyl)amino]methyl]pyrrolidine-1-carboxylate (0.500 g, 1.15 mmol) in DCM (4 mL) was added TFA (1 mL) at room temperature. The reaction was stirred for 1 h and concentrated under reduced pressure. The residue was purified by reverse phase chromatography, eluting with 50% ACN in water (plus 0.05% TFA) to afford [[(2S,4R)-4-(2,3-dichloro-6-methoxyphenyl)pyrrolidin-2-yl]methyl](2-methoxyethyl)amine as a light yellow oil (0.400 g, 73%): LCMS (ESI) calc'd C₁₅H₂₂Cl₂N₂O₂ for [M+H]⁺: 333, 335 (3:2) found 333, 335 (3:2).

Step c:

To a stirred solution of [[(2S,4R)-4-(2,3-dichloro-6-methoxyphenyl)pyrrolidin-2-yl]methyl](2-methoxyethyl)amine (0.300 g, 0.90 mmol) in ACN (3 mL) was added CDI (0.100 g, 0.63 mmol) at 0° C. The reaction was stirred at 0° C. for 12 h, quenched with water and concentrated under reduced pressure. The residue was purified by reverse phase chromatography, eluting with 50% ACN in water (plus 0.05% TFA) to afford (6R,7aS)-6-(2,3-dichloro-6-methoxyphenyl)-2-(2-methoxyethyl)-tetrahydro-1H-pyrrolo[1,2-c]imidazol-3-one as a colorless oil (0.160 g, 49%): LCMS (ESI) calc'd C₁₆H₂OCl₂N₂O₃ for [M+H]⁺: 359, 361 (3:2) found 359, 361 (3:2).

Step d:

To a stirred solution of (6R,7aS)-6-(2,3-dichloro-6-methoxyphenyl)-2-(2-methoxyethyl)-tetrahydro-1H-pyrrolo[1,2-c]imidazol-3-one (80.0 mg, 0.22 mmol) in DCM (1 mL) was added BBr₃ (0.560 g, 2.22 mmol) at room temperature. The reaction was stirred for 1 h, quenched with saturated aq. NH₄HCO₃ (20 mL) followed by extraction with EA (3×20 mL). The combined organic layers were washed with brine (3×20 mL) and dried over anhydrous Na₂SO₄. After filtration, the filtrate was concentrated under reduced pressure. The residue was purified by Prep-HPLC with the following conditions Column: X Bridge Shield RP18 OBD Column, 19×250 mm, 10 m; Mobile Phase A: Water (plus 10 mM NH₄HCO₃), Mobile Phase B: ACN; Flow rate: 20 mL/min; Gradient: 40% B to 70% B in 5.5 min; Detector UV 254/210 nm; Retention Time: 5.53 min. The fractions containing the desired product were collected and concentrated under reduced pressure to afford Compound 290 ((6R,7aS)-6-(2,3-dichloro-6-hydroxyphenyl)-2-(2-hydroxyethyl)-tetrahydro-1H-pyrrolo[1,2-c]imidazol-3-one as an off-white solid (13.0 mg, 17%): LCMS (ESI) calc'd C₁₄H₁₆Cl₂N₂O₃ for [M+H]⁺: 331, 333 (3:2) found 331, 333 (3:2): ¹H NMR (400 MHz, CD₃OD) δ 7.20 (d, J=8.8 Hz, 1H), 6.71 (d, J=8.8 Hz, 1H), 4.38-4.19 (m, 1H), 4.01-3.87 (m, 2H), 3.83-3.63 (m, 3H), 3.54 (dd, J=9.4, 2.2 Hz, 1H), 3.39-3.35 (m, 2H), 3.30-3.24 (m, 1H), 2.29-2.23 (m, 1H), 2.06-1.90 (m, 1H).

Example 108. Compounds 291-293 were prepared in an analogous fashion as that described for Compound 290.

| Compound | | | |
|---|---|---|---|
| Number | Structure | Chemical Name | MS: (M + H)⁺ & ¹H MNR |
| 291 | 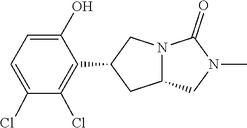 [SMILES: CN1C[C@@H]2C[C@H](c3c(O)ccc(Cl)c3Cl)CN2C1=O] | (6R,7aS)-6-(2,3- dichloro-6- hydroxyphenyl)-2- methyl-tetrahydro- 1H-pyrrolo[1,2- c]imidazol-3-one | [M + H]⁺: 301, 303 (3:2); ¹H NMR (400 MHz, CD₃OD) δ 7.21 (d, J = 8.8 Hz, 1H), 6.70 (d, J = 8.9 Hz, 1H), 4.32-4.24 (m, 1H), 3.95-3.88 (m, 2H), 3.60 (dd, J = 9.4, 8.5 Hz, 1H), 3.43 (dd, J = 9.4, 2.3 Hz, 1H), 3.28 (dd, J = 10.6, 9.9 Hz, 1H), 2.85 (s, 3H), 2.28- |
| | | | 2.20 (m, 1H), 2.06-1.98 (m, 1H). |
| 292 | 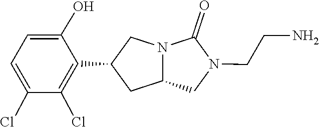 [SMILES: NCCN1C[C@@H]2C[C@H](c3c(O)ccc(Cl)c3Cl)CN2C1=O] | (6R,7aS)-2-(2- aminoethyl)-6-(2,3- dichloro-6- hydroxyphenyl)- tetrahydro-1H- pyrrolo[1,2- c]imidazol-3-one | [M + H]⁺: 330, 332 (3:2); ¹H NMR (400 MHz, CD₃OD) δ 7.23 (d, J = 8.8 Hz, 1H), 6.73 (d, J = 8.7 Hz, 1H), 4.37-4.28 (m, 1H), 4.06-3.94 (m, 2H), 3.78-3.74 (m, 1H), 3.68-3.59 (m, 1H), 4.49-3.39 (m, 2H), 3.36-3.33 (m, 1H), 3.20- 3.10 (m, 2H), 2.27-2.18 (m, 1H), 2.13-2.05 (m, 1H). |
| 293 | 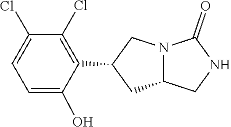 [SMILES: O=C1NC[C@@H]2C[C@H](c3c(O)ccc(Cl)c3Cl)CN12] | (6R,7aS)-6-(2,3- dichloro-6- hydroxyphenyl)- hexahydropyrrolo[1, 2-c]imidazol-3-one | [M + H]⁺: 287, 289 (3:2); ¹H NMR (400 MHz, CD₃OD) δ 7.21 (d, J = 8.7 Hz, 1H), 6.72 (d, J = 8.8 Hz, 1H), 4.35-4.23 (m, 1H), 4.09-3.91 (m, 2H), 3.71-3.63 (m, 1H), 3.43-3.22 (m, 2H), 2.37-2.27 (m, 1H), 2.05-1.93 (m, 1H). |

### Example 109. Evaluation of Kv1.3 Potassium Channel Blocker Activities

This assay is used to evaluate the disclosed compounds' activities as Kv1.3 potassium channel blockers.

### Cell Culture

CHO-K1 cells stably expressing Kv1.3 were grown in DMEM containing 10% heat-inactivated FBS, 1 mM sodium pyruvate, 2 mM L-glutamine and G418 (500 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM glucose, and 10 mM HEPES; pH adjusted to 7.4 with NaOH; 295-305 mOsm. The internal solution contained 50 mM KCl, 10 mM NaCl, 60 mM KF, 20 mM EGTA, and 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The currents were evoked by applying 100 ms depolarizing pulses from −90 mV (holding potential) to +40 mV were applied with 0.1 Hz frequency. Control (compound-free) and compound pulse trains for each compound concentration applied contained 20 pulses.

10-second breaks were used between pulse trains (see Table 2 below).

### Table 2. Voltage Protocol.

Patch clamp recordings and compound application

Whole-cell current recordings and compound application were enabled by means of an automated patch clamp platform Patchliner (Nanion Technologies GmbH). EPC 10 patch clamp amplifier (HEKA Elektronik Dr. Schulze GmbH) along with Patchmaster software (HEKA Elektronik Dr. Schulze GmbH) was used for data acquisition. Data were sampled at 10 kHz without filtering. Passive leak currents were subtracted online using a P/4 procedure (HEKA Elektronik Dr. Schulze GmbH). Increasing compound concentrations were applied consecutively to the same cell without washouts in between. Total compound incubation time before the next pulse train was not longer than 10 seconds. Peak current inhibition was observed during compound equilibration.

### Data Analysis

AUC and peak values were obtained with Patchmaster (HEKA Elektronik Dr. Schulze GmbH). To determine IC₅₀, the last single pulse in the pulse train corresponding to a given compound concentration was used. Obtained AUC and peak values in the presence of compound were normalized to control values in the absence of compound. Using Origin (OridinLab), IC₅₀ was derived from data fit to Hill equation: I_{compound}/I_{control}=(100−A)/(1+([compound]/IC₅₀)nH)+A, where IC₅₀ value is the concentration at which current inhibition is half-maximal, [compound] is the applied compound concentration, A is the fraction of current that is not blocked and nH is the Hill coefficient.

### Example 110. Evaluation of hERG Activities

This assay is used to evaluate the disclosed compounds' inhibition activities against the hERG channel.

hERG Electrophysiology

This assay is used to evaluate the disclosed compounds' inhibition activities against the hERG channel.

### Cell Culture

CHO-K1 cells stably expressing hERG were grown in Ham's F-12 Medium with glutamine containing 10% heat-inactivated FBS, 1% penicillin/streptomycin, hygromycin (100 μg/ml) and G418 (100 μg/ml). Cells were grown in culture flasks at 37° C. in a 5% CO₂-humidified incubator.

### Solutions

The cells were bathed in an extracellular solution containing 140 mM NaCl, 4 mM KCl, 2 mM CaCl₂), 1 mM MgCl₂, 5 mM glucose, and 10 mM HEPES; pH adjusted to 7.4 with NaOH; 295-305 mOsm. The internal solution contained 50 mM KCl, 10 mM NaCl, 60 mM KF, 20 mM EGTA, and 10 mM HEPES; pH adjusted to 7.2 with KOH; 285 mOsm. All compounds were dissolved in DMSO at 30 mM. Compound stock solutions were freshly diluted with external solution to concentrations of 30 nM, 100 nM, 300 nM, 1 μM, 3 μM, 10 μM, 30 μM and 100 μM. The highest content of DMSO (0.3%) was present in 100 μM.

### Voltage Protocol

The voltage protocol (see Table 3) was designed to simulate voltage changes during a cardiac action potential with a 300 ms depolarization to +20 mV (analogous to the plateau phase of the cardiac action potential), a repolarization for 300 ms to −50 mV (inducing a tail current) and a final step to the holding potential of −80 mV. The pulse frequency was 0.3 Hz. Control (compound-free) and compound pulse trains for each compound concentration applied contained 70 pulses.

### Table 3. hERG Voltage Protocol.

### Patch Clamp Recordings and Compound Application

Whole-cell current recordings and compound application were enabled by means of an automated patch clamp platform Patchliner (Nanion). EPC 10 patch clamp amplifier (HEKA) along with Patchmaster software (HEKA Elektronik Dr. Schulze GmbH) was used for data acquisition. Data were sampled at 10 kHz without filtering. Increasing compound concentrations were applied consecutively to the same cell without washouts in between.

### Data Analysis

AUC and PEAK values were obtained with Patchmaster (HEKA Elektronik Dr. Schulze GmbH). To determine IC₅₀ the last single pulse in the pulse train corresponding to a given compound concentration was used. Obtained AUC and PEAK values in the presence of compound were normalized to control values in the absence of compound. Using Origin (OridinLab), IC₅₀ was derived from data fit to Hill equation: I_{compound}/I_{control}=(100-A)/(1+([compound]/IC₅₀)nH)+A, where IC₅₀ is the concentration at which current inhibition is half-maximal, [compound] is the applied compound concentration, A is the fraction of current that is not blocked and nH is the Hill coefficient.

Tables 4 and 5 provide a summary of the inhibition activities of certain selected compounds against Kv1.3 potassium channel and hERG channel.

TABLE 4
IC₅₀ (μM) values of certain exemplified compounds against
Kv1.3 potassium channel and hERG channel

| Compound | | | hERG |
|---|---|---|---|
| Number | Structure | Kv1.3 IC₅₀ | IC₅₀ |
| 1 | 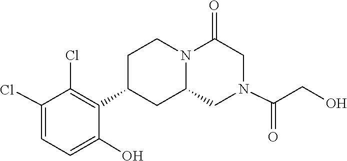 [SMILES: O=C(CO)N1CC(=O)N2CC[C@@H](c3c(O)ccc(Cl)c3Cl)C[C@H]2C1] | <1 | >30 |
| 2 | 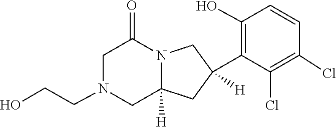 [SMILES: O=C1CN(CCO)C[C@@H]2C[C@H](c3c(O)ccc(Cl)c3Cl)CN12] | <1 | >30 |
| 3 | 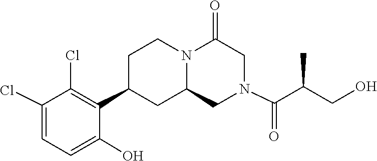 [SMILES: C[C@@H](CO)C(=O)N1CC(=O)N2CC[C@H](c3c(O)ccc(Cl)c3Cl)C[C@@H]2C1] | <1 | * |
| 4 | 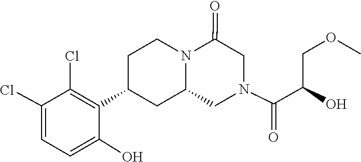 [SMILES: COC[C@@H](O)C(=O)N1CC(=O)N2CC[C@@H](c3c(O)ccc(Cl)c3Cl)C[C@H]2C1] | <1 | >30 |
| 5 | 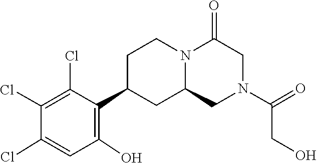 [SMILES: O=C(CO)N1CC(=O)N2CC[C@H](c3c(O)cc(Cl)c(Cl)c3Cl)C[C@@H]2C1] | <1 | * |
| 6 | 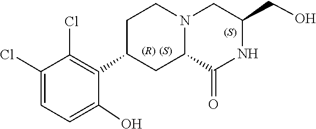 | <10 | * |
| 7 | 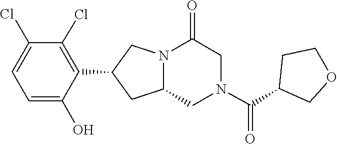 [SMILES: O=C([C@@H]1CCOC1)N1CC(=O)N2C[C@@H](c3c(O)ccc(Cl)c3Cl)C[C@H]2C1] | <1 | <30 |
| 8 | 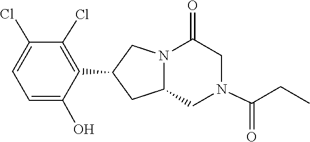 [SMILES: CCC(=O)N1CC(=O)N2C[C@@H](c3c(O)ccc(Cl)c3Cl)C[C@H]2C1] | <1 | <30 |
| 9 | 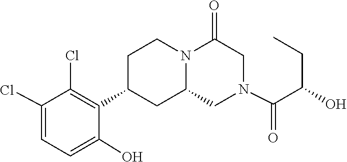 [SMILES: CC[C@H](O)C(=O)N1CC(=O)N2CC[C@@H](c3c(O)ccc(Cl)c3Cl)C[C@H]2C1] | <1 | >30 |
| 10 | 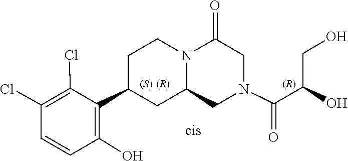 | <1 | >30 |
| 11 | 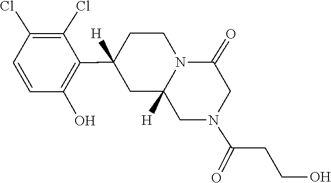 [SMILES: O=C(CCO)N1CC(=O)N2CC[C@@H](c3c(O)ccc(Cl)c3Cl)C[C@H]2C1] | <1 | >30 |
| 12 | 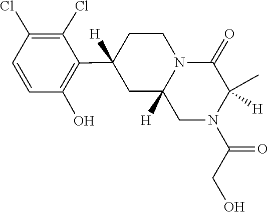 [SMILES: C[C@@H]1C(=O)N2CC[C@@H](c3c(O)ccc(Cl)c3Cl)C[C@H]2CN1C(=O)CO] | <10 | * |
| 13 | 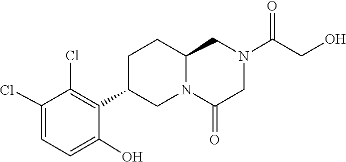 [SMILES: O=C(CO)N1CC(=O)N2C[C@H](c3c(O)ccc(Cl)c3Cl)CC[C@H]2C1] | <1 | * |
| 14 | 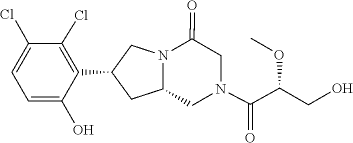 [SMILES: CO[C@H](CO)C(=O)N1CC(=O)N2C[C@@H](c3c(O)ccc(Cl)c3Cl)C[C@H]2C1] | <1 | >30 |
| 15 | 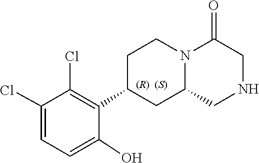 | <1 | <30 |
| 16 | 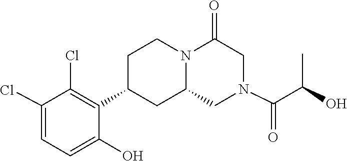 [SMILES: C[C@@H](O)C(=O)N1CC(=O)N2CC[C@@H](c3c(O)ccc(Cl)c3Cl)C[C@H]2C1] | <1 | >30 |
| 17 | 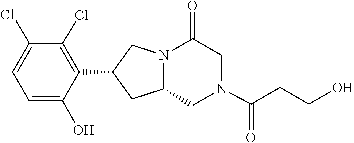 [SMILES: O=C(CCO)N1CC(=O)N2C[C@@H](c3c(O)ccc(Cl)c3Cl)C[C@H]2C1] | <1 | >30 |
| 18 | 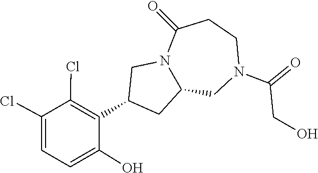 [SMILES: O=C(CO)N1CCC(=O)N2C[C@@H](c3c(O)ccc(Cl)c3Cl)C[C@H]2C1] | <1 | >30 |
| 19 | 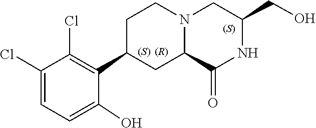 | <10 | * |
| 20 | 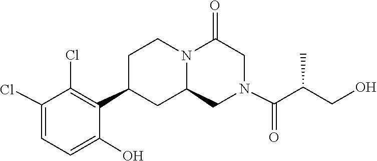 [SMILES: C[C@H](CO)C(=O)N1CC(=O)N2CC[C@H](c3c(O)ccc(Cl)c3Cl)C[C@@H]2C1] | <1 | * |
| 21 | 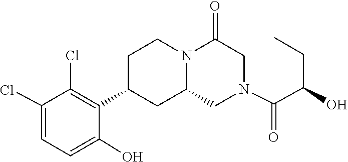 [SMILES: CC[C@@H](O)C(=O)N1CC(=O)N2CC[C@@H](c3c(O)ccc(Cl)c3Cl)C[C@H]2C1] | <1 | <30 |
| 22 | 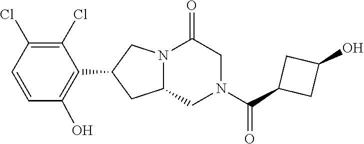 [SMILES: O=C1CN(C(=O)[C@H]2C[C@@H](O)C2)C[C@@H]2C[C@H](c3c(O)ccc(Cl)c3Cl)CN12] | <1 | >30 |
| 23 | 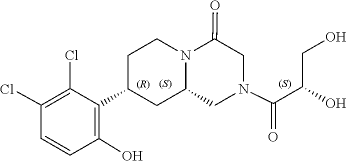 | <1 | >30 |
| 24 | 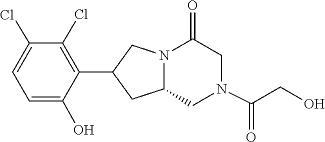 [SMILES: O=C(CO)N1CC(=O)N2CC(c3c(O)ccc(Cl)c3Cl)C[C@H]2C1] | <1 | >30 |
| 25 | 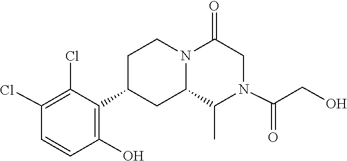 | <1 | >30 |
| 26 | 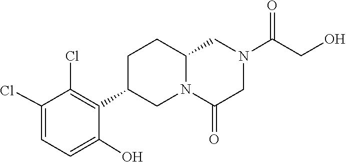 [SMILES: O=C(CO)N1CC(=O)N2C[C@H](c3c(O)ccc(Cl)c3Cl)CC[C@@H]2C1] | <10 | * |
| 27 | 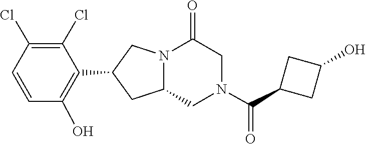 [SMILES: O=C1CN(C(=O)[C@H]2C[C@H](O)C2)C[C@@H]2C[C@H](c3c(O)ccc(Cl)c3Cl)CN12] | <1 | >30 |
| 28 | 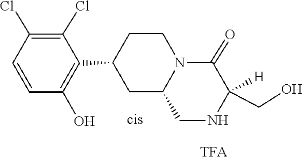 [SMILES: O=C1[C@@H](CO)NC[C@@H]2C[C@H](c3c(O)ccc(Cl)c3Cl)CCN12] | <1 | >30 |
| 29 | 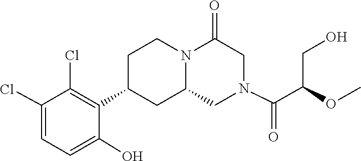 [SMILES: CO[C@H](CO)C(=O)N1CC(=O)N2CC[C@@H](c3c(O)ccc(Cl)c3Cl)C[C@H]2C1] | <1 | >30 |
| 30 | 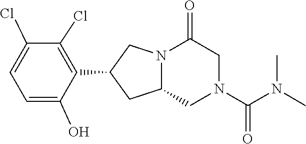 [SMILES: CN(C)C(=O)N1CC(=O)N2C[C@@H](c3c(O)ccc(Cl)c3Cl)C[C@H]2C1] | <1 | * |
| 31 | 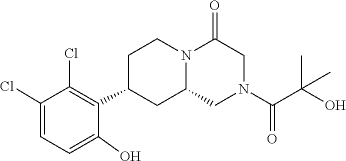 [SMILES: CC(C)(O)C(=O)N1CC(=O)N2CC[C@@H](c3c(O)ccc(Cl)c3Cl)C[C@H]2C1] | <1 | * |
| 32 | 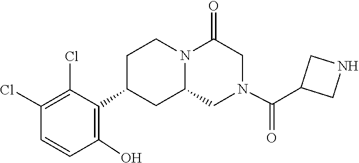 [SMILES: O=C(C1CNC1)N1CC(=O)N2CC[C@@H](c3c(O)ccc(Cl)c3Cl)C[C@H]2C1] | <1 | >30 |
| 33 | 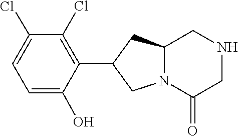 | <1 | <30 |
| 34 | 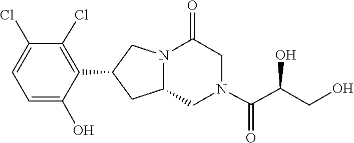 [SMILES: O=C([C@@H](O)CO)N1CC(=O)N2C[C@@H](c3c(O)ccc(Cl)c3Cl)C[C@H]2C1] | <1 | >30 |
| 35 | 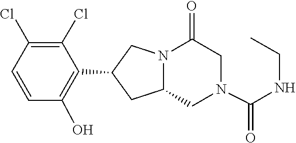 [SMILES: CCNC(=O)N1CC(=O)N2C[C@@H](c3c(O)ccc(Cl)c3Cl)C[C@H]2C1] | <1 | >30 |
| 36 | 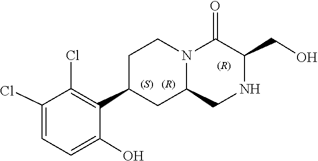 | <1 | * |
| 37 | 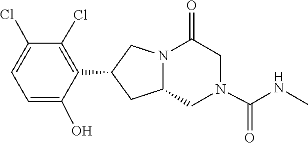 [SMILES: CNC(=O)N1CC(=O)N2C[C@@H](c3c(O)ccc(Cl)c3Cl)C[C@H]2C1] | <1 | >30 |
| 38 | 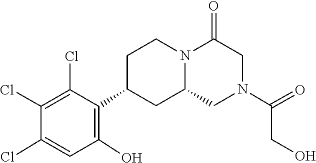 [SMILES: O=C(CO)N1CC(=O)N2CC[C@@H](c3c(O)cc(Cl)c(Cl)c3Cl)C[C@H]2C1] | <1 | >30 |
| 39 | 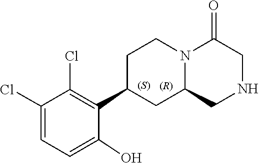 | <1 | * |
| 40 | 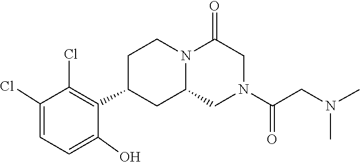 [SMILES: CN(C)CC(=O)N1CC(=O)N2CC[C@@H](c3c(O)ccc(Cl)c3Cl)C[C@H]2C1] | <1 | <30 |
| 41 | 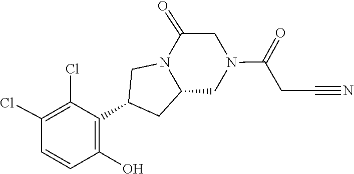 [SMILES: N#CCC(=O)N1CC(=O)N2C[C@@H](c3c(O)ccc(Cl)c3Cl)C[C@H]2C1] | <1 | >30 |
| 42 | 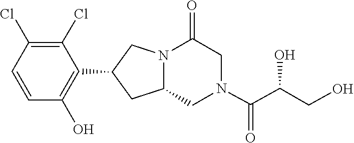 [SMILES: O=C([C@H](O)CO)N1CC(=O)N2C[C@@H](c3c(O)ccc(Cl)c3Cl)C[C@H]2C1] | <1 | >30 |
| 43 | 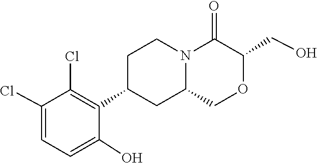 [SMILES: O=C1[C@H](CO)OC[C@@H]2C[C@H](c3c(O)ccc(Cl)c3Cl)CCN12] | <1 | * |
| 44 | 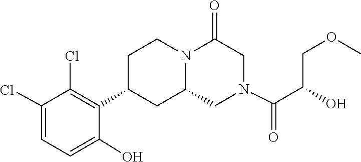 [SMILES: COC[C@H](O)C(=O)N1CC(=O)N2CC[C@@H](c3c(O)ccc(Cl)c3Cl)C[C@H]2C1] | <1 | >30 |
| 45 | 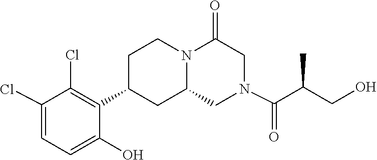 [SMILES: C[C@@H](CO)C(=O)N1CC(=O)N2CC[C@@H](c3c(O)ccc(Cl)c3Cl)C[C@H]2C1] | <1 | >30 |
| 46 | 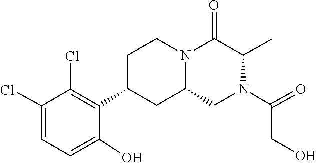 [SMILES: C[C@H]1C(=O)N2CC[C@@H](c3c(O)ccc(Cl)c3Cl)C[C@H]2CN1C(=O)CO] | <1 | >30 |
| 47 | 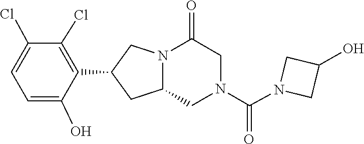 [SMILES: O=C(N1CC(O)C1)N1CC(=O)N2C[C@@H](c3c(O)ccc(Cl)c3Cl)C[C@H]2C1] | <1 | >30 |
| 48 | 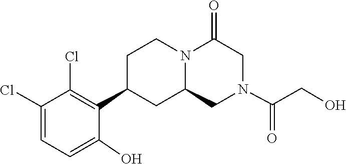 [SMILES: O=C(CO)N1CC(=O)N2CC[C@H](c3c(O)ccc(Cl)c3Cl)C[C@@H]2C1] | <1 | * |
| 49 | 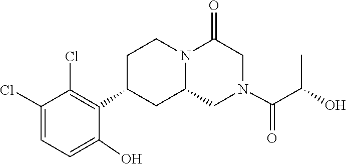 [SMILES: C[C@H](O)C(=O)N1CC(=O)N2CC[C@@H](c3c(O)ccc(Cl)c3Cl)C[C@H]2C1] | <1 | >30 |
| 50 | 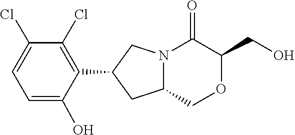 [SMILES: O=C1[C@@H](CO)OC[C@@H]2C[C@H](c3c(O)ccc(Cl)c3Cl)CN12] | <1 | >30 |
| 51 | 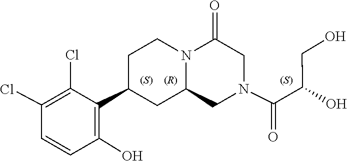 | <1 | * |
| 52 | 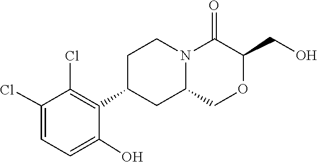 [SMILES: O=C1[C@@H](CO)OC[C@@H]2C[C@H](c3c(O)ccc(Cl)c3Cl)CCN12] | <1 | >30 |
| 53 | 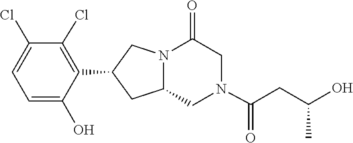 [SMILES: C[C@@H](O)CC(=O)N1CC(=O)N2C[C@@H](c3c(O)ccc(Cl)c3Cl)C[C@H]2C1] | <1 | * |
| 54 | 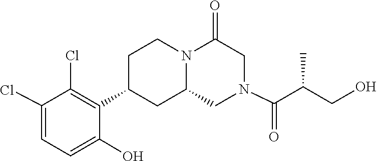 [SMILES: C[C@H](CO)C(=O)N1CC(=O)N2CC[C@@H](c3c(O)ccc(Cl)c3Cl)C[C@H]2C1] | <1 | >30 |
| 55 | 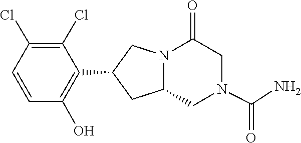 [SMILES: NC(=O)N1CC(=O)N2C[C@@H](c3c(O)ccc(Cl)c3Cl)C[C@H]2C1] | <1 | >30 |
| 56 | 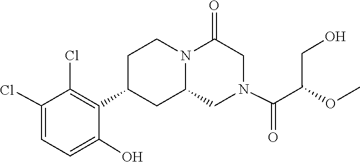 [SMILES: CO[C@@H](CO)C(=O)N1CC(=O)N2CC[C@@H](c3c(O)ccc(Cl)c3Cl)C[C@H]2C1] | <1 | * |
| 57 | 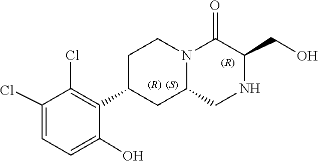 | <1 | >30 |
| 58 | 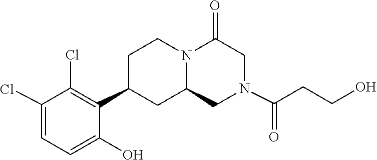 [SMILES: O=C(CCO)N1CC(=O)N2CC[C@H](c3c(O)ccc(Cl)c3Cl)C[C@@H]2C1] | <1 | * |
| 59 | 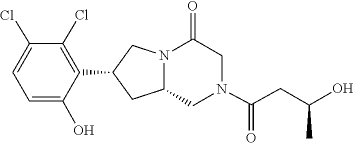 [SMILES: C[C@H](O)CC(=O)N1CC(=O)N2C[C@@H](c3c(O)ccc(Cl)c3Cl)C[C@H]2C1] | <1 | >30 |
| 60 | 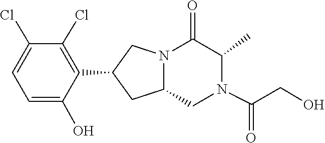 [SMILES: C[C@H]1C(=O)N2C[C@@H](c3c(O)ccc(Cl)c3Cl)C[C@H]2CN1C(=O)CO] | <1 | * |
| 61 | 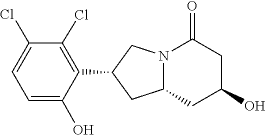 [SMILES: O=C1C[C@@H](O)C[C@@H]2C[C@H](c3c(O)ccc(Cl)c3Cl)CN12] | <1 | >30 |
| 62 | 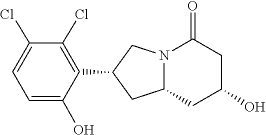 [SMILES: O=C1C[C@H](O)C[C@@H]2C[C@H](c3c(O)ccc(Cl)c3Cl)CN12] | <1 | >30 |

*Not Tested.

TABLE 5
IC₅₀ (μM) values of certain exemplified compounds against
Kv1.3 potassium channel and hERG channel.

| | | Kv1.3 | hERG |
|---|---|---|---|
| Compound Number | Structure | IC₅₀ | IC₅₀ |
| 63 | 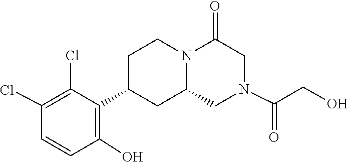 [SMILES: O=C(CO)N1CC(=O)N2CC[C@@H](c3c(O)ccc(Cl)c3Cl)C[C@H]2C1] | <1 | <30 |
| 64 | 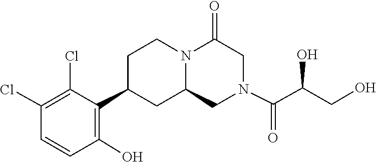 [SMILES: O=C([C@@H](O)CO)N1CC(=O)N2CC[C@H](c3c(O)ccc(Cl)c3Cl)C[C@@H]2C1] | <1 | >30 |
| 65 | 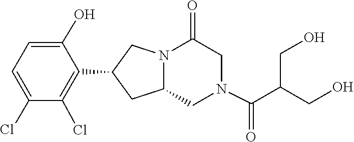 [SMILES: O=C(C(CO)CO)N1CC(=O)N2C[C@@H](c3c(O)ccc(Cl)c3Cl)C[C@H]2C1] | <1 | >30 |
| 66 | 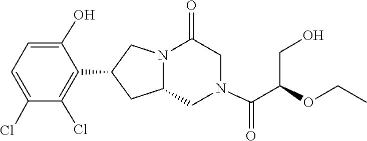 [SMILES: CCO[C@H](CO)C(=O)N1CC(=O)N2C[C@@H](c3c(O)ccc(Cl)c3Cl)C[C@H]2C1] | <1 | >30 |
| 67 | 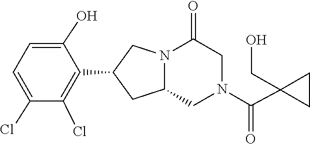 [SMILES: O=C1CN(C(=O)C2(CO)CC2)C[C@@H]2C[C@H](c3c(O)ccc(Cl)c3Cl)CN12] | <1 | >30 |
| 68 | 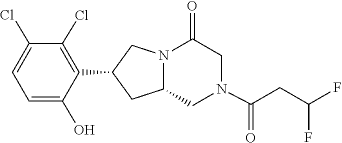 [SMILES: O=C(CC(F)F)N1CC(=O)N2C[C@@H](c3c(O)ccc(Cl)c3Cl)C[C@H]2C1] | <1 | >30 |
| 69 | 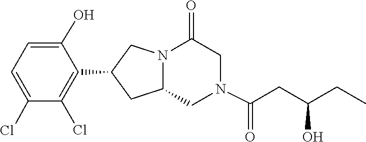 [SMILES: CC[C@@H](O)CC(=O)N1CC(=O)N2C[C@@H](c3c(O)ccc(Cl)c3Cl)C[C@H]2C1] | <1 | >30 |
| 70 | 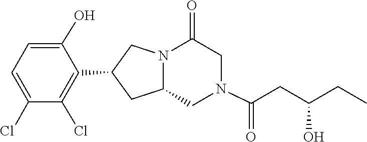 [SMILES: CC[C@H](O)CC(=O)N1CC(=O)N2C[C@@H](c3c(O)ccc(Cl)c3Cl)C[C@H]2C1] | <1 | <30 |
| 71 | 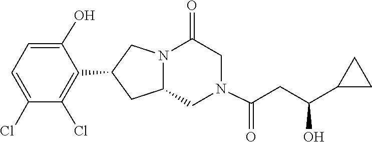 [SMILES: O=C(C[C@H](O)C1CC1)N1CC(=O)N2C[C@@H](c3c(O)ccc(Cl)c3Cl)C[C@H]2C1] | <1 | <30 |
| 72 | 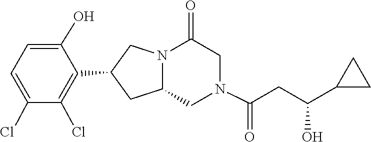 [SMILES: O=C(C[C@@H](O)C1CC1)N1CC(=O)N2C[C@@H](c3c(O)ccc(Cl)c3Cl)C[C@H]2C1] | <1 | <30 |
| 73 | 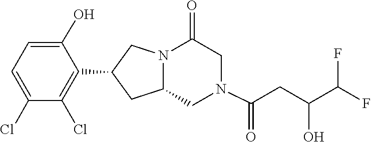 [SMILES: O=C(CC(O)C(F)F)N1CC(=O)N2C[C@@H](c3c(O)ccc(Cl)c3Cl)C[C@H]2C1] | <1 | >30 |
| 74 | 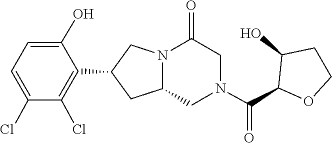 [SMILES: O=C([C@@H]1OCC[C@@H]1O)N1CC(=O)N2C[C@@H](c3c(O)ccc(Cl)c3Cl)C[C@H]2C1] | <1 | >30 |
| 75 | 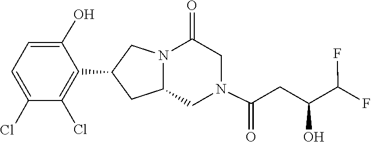 [SMILES: O=C(C[C@H](O)C(F)F)N1CC(=O)N2C[C@@H](c3c(O)ccc(Cl)c3Cl)C[C@H]2C1] | <1 | >30 |
| 76 | 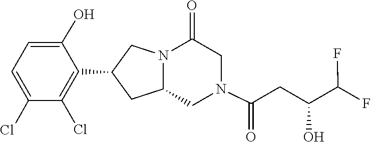 [SMILES: O=C(C[C@@H](O)C(F)F)N1CC(=O)N2C[C@@H](c3c(O)ccc(Cl)c3Cl)C[C@H]2C1] | <1 | >30 |
| 77 | 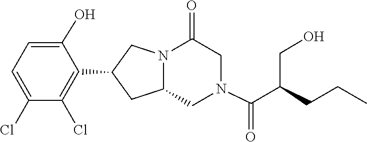 [SMILES: CCC[C@H](CO)C(=O)N1CC(=O)N2C[C@@H](c3c(O)ccc(Cl)c3Cl)C[C@H]2C1] | <1 | >30 |
| 78 | 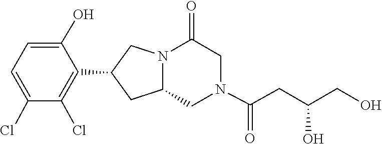 [SMILES: O=C(C[C@@H](O)CO)N1CC(=O)N2C[C@@H](c3c(O)ccc(Cl)c3Cl)C[C@H]2C1] | <1 | >30 |
| 83 | 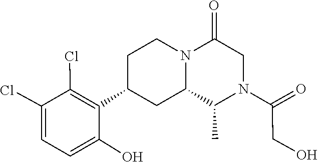 [SMILES: C[C@@H]1[C@@H]2C[C@H](c3c(O)ccc(Cl)c3Cl)CCN2C(=O)CN1C(=O)CO] | <10 | * |
| 84 | 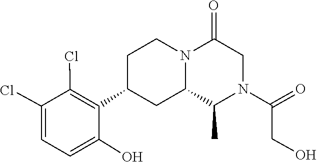 [SMILES: C[C@H]1[C@@H]2C[C@H](c3c(O)ccc(Cl)c3Cl)CCN2C(=O)CN1C(=O)CO] | <1 | >30 |
| 85 | 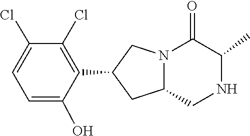 [SMILES: C[C@@H]1NC[C@@H]2C[C@H](c3c(O)ccc(Cl)c3Cl)CN2C1=O] | <1 | <30 |
| 87 | 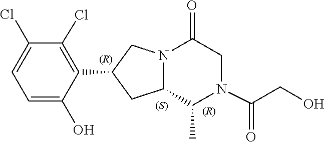 | <1 | * |
| 88 | 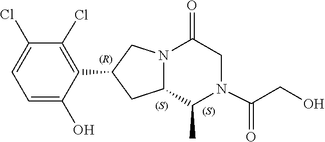 | <1 | >30 |
| 90 | 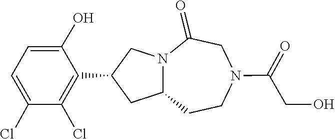 [SMILES: O=C(CO)N1CC[C@@H]2C[C@H](c3c(O)ccc(Cl)c3Cl)CN2C(=O)C1] | <1 | * |
| 91 | 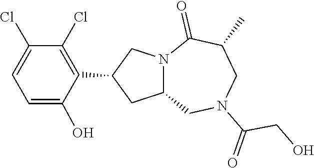 [SMILES: C[C@@H]1CN(C(=O)CO)C[C@@H]2C[C@H](c3c(O)ccc(Cl)c3Cl)CN2C1=O] | <1 | >30 |
| 92 | 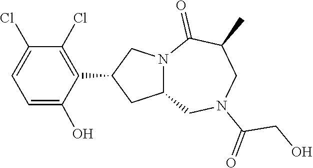 [SMILES: C[C@H]1CN(C(=O)CO)C[C@@H]2C[C@H](c3c(O)ccc(Cl)c3Cl)CN2C1=O] | <1 | >30 |
| 93 | 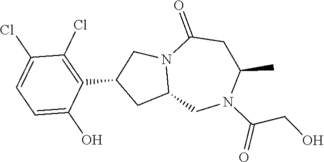 [SMILES: C[C@@H]1CC(=O)N2C[C@@H](c3c(O)ccc(Cl)c3Cl)C[C@H]2CN1C(=O)CO] | <1 | <30 |
| 94 | 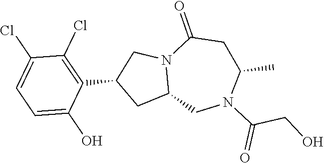 [SMILES: C[C@H]1CC(=O)N2C[C@@H](c3c(O)ccc(Cl)c3Cl)C[C@H]2CN1C(=O)CO] | <1 | >30 |
| 96 | 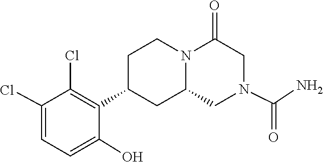 [SMILES: NC(=O)N1CC(=O)N2CC[C@@H](c3c(O)ccc(Cl)c3Cl)C[C@H]2C1] | <1 | >30 |
| 97 | 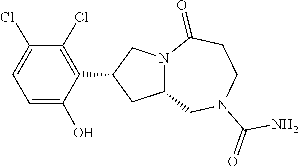 [SMILES: NC(=O)N1CCC(=O)N2C[C@@H](c3c(O)ccc(Cl)c3Cl)C[C@H]2C1] | <1 | >30 |
| 99 | 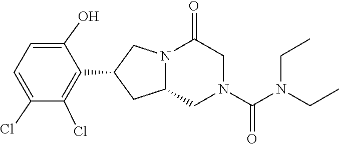 [SMILES: CCN(CC)C(=O)N1CC(=O)N2C[C@@H](c3c(O)ccc(Cl)c3Cl)C[C@H]2C1] | <1 | * |
| 100 | 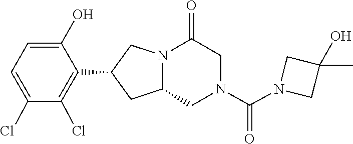 [SMILES: CC1(O)CN(C(=O)N2CC(=O)N3C[C@@H](c4c(O)ccc(Cl)c4Cl)C[C@H]3C2)C1] | <1 | >30 |
| 101 | 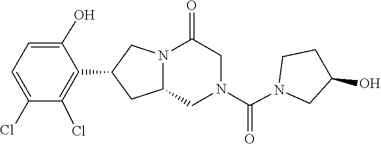 [SMILES: O=C(N1CC[C@@H](O)C1)N1CC(=O)N2C[C@@H](c3c(O)ccc(Cl)c3Cl)C[C@H]2C1] | <1 | >30 |
| 102 | 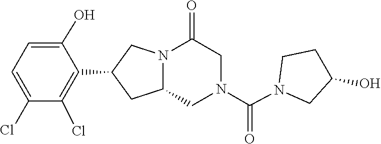 [SMILES: O=C(N1CC[C@H](O)C1)N1CC(=O)N2C[C@@H](c3c(O)ccc(Cl)c3Cl)C[C@H]2C1] | <1 | >30 |
| 103 | 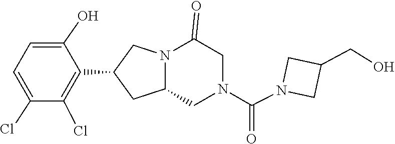 [SMILES: O=C(N1CC(CO)C1)N1CC(=O)N2C[C@@H](c3c(O)ccc(Cl)c3Cl)C[C@H]2C1] | <1 | >30 |
| 104 | 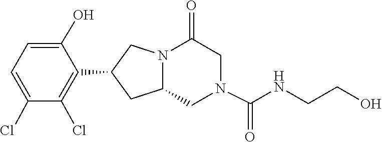 [SMILES: O=C(NCCO)N1CC(=O)N2C[C@@H](c3c(O)ccc(Cl)c3Cl)C[C@H]2C1] | <1 | >30 |
| 109 | 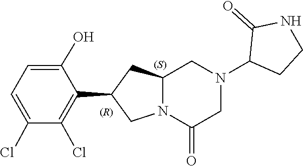 | <1 | * |
| 110 | 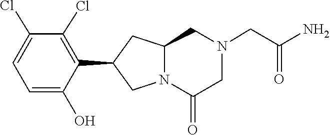 [SMILES: NC(=O)CN1CC(=O)N2C[C@@H](c3c(O)ccc(Cl)c3Cl)C[C@H]2C1] | <1 | >30 |
| 111 | 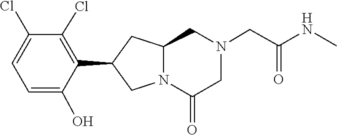 [SMILES: CNC(=O)CN1CC(=O)N2C[C@@H](c3c(O)ccc(Cl)c3Cl)C[C@H]2C1] | <1 | >30 |
| 112 | 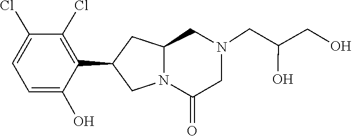 [SMILES: O=C1CN(CC(O)CO)C[C@@H]2C[C@H](c3c(O)ccc(Cl)c3Cl)CN12] | <1 | >30 |
| 113 | 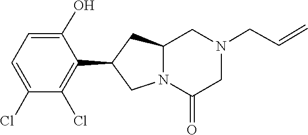 [SMILES: C=CCN1CC(=O)N2C[C@@H](c3c(O)ccc(Cl)c3Cl)C[C@H]2C1] | <1 | * |
| 114 | 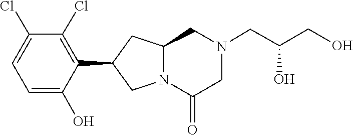 [SMILES: O=C1CN(C[C@@H](O)CO)C[C@@H]2C[C@H](c3c(O)ccc(Cl)c3Cl)CN12] | <1 | >30 |
| 115 | 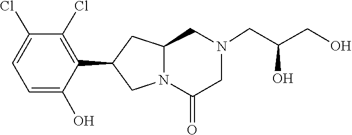 [SMILES: O=C1CN(C[C@H](O)CO)C[C@@H]2C[C@H](c3c(O)ccc(Cl)c3Cl)CN12] | <1 | >30 |
| 116 | 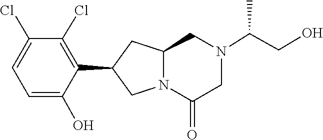 [SMILES: C[C@H](CO)N1CC(=O)N2C[C@@H](c3c(O)ccc(Cl)c3Cl)C[C@H]2C1] | <1 | >30 |
| 117 | 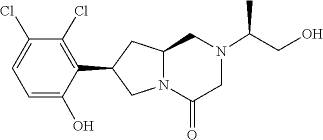 [SMILES: C[C@@H](CO)N1CC(=O)N2C[C@@H](c3c(O)ccc(Cl)c3Cl)C[C@H]2C1] | <1 | >30 |
| 118 | 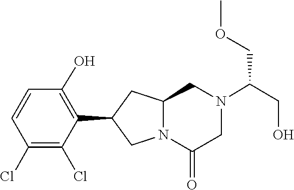 [SMILES: COC[C@H](CO)N1CC(=O)N2C[C@@H](c3c(O)ccc(Cl)c3Cl)C[C@H]2C1] | <1 | >30 |
| 119 | 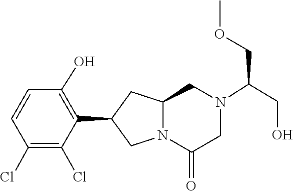 [SMILES: COC[C@@H](CO)N1CC(=O)N2C[C@@H](c3c(O)ccc(Cl)c3Cl)C[C@H]2C1] | <1 | >30 |
| 120 | 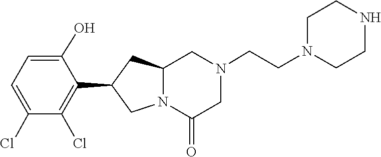 [SMILES: O=C1CN(CCN2CCNCC2)C[C@@H]2C[C@H](c3c(O)ccc(Cl)c3Cl)CN12] | <1 | >30 |
| 121 | 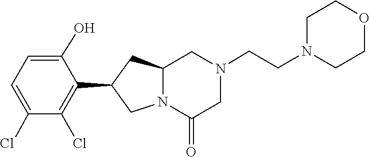 [SMILES: O=C1CN(CCN2CCOCC2)C[C@@H]2C[C@H](c3c(O)ccc(Cl)c3Cl)CN12] | <1 | <30 |
| 122 | 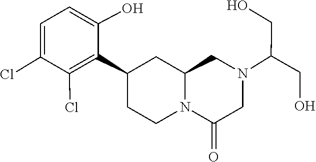 [SMILES: O=C1CN(C(CO)CO)C[C@@H]2C[C@H](c3c(O)ccc(Cl)c3Cl)CCN12] | <1 | >30 |
| 123 | 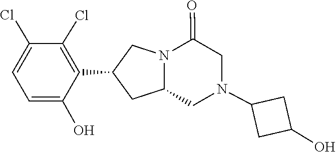 [SMILES: O=C1CN(C2CC(O)C2)C[C@@H]2C[C@H](c3c(O)ccc(Cl)c3Cl)CN12] | <1 | >30 |
| 124 | 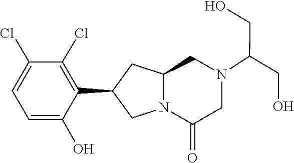 [SMILES: O=C1CN(C(CO)CO)C[C@@H]2C[C@H](c3c(O)ccc(Cl)c3Cl)CN12] | <1 | >30 |
| 125 | 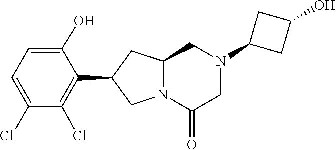 [SMILES: O=C1CN([C@H]2C[C@H](O)C2)C[C@@H]2C[C@H](c3c(O)ccc(Cl)c3Cl)CN12] | <1 | >30 |
| 126 | 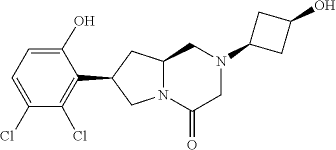 [SMILES: O=C1CN([C@H]2C[C@@H](O)C2)C[C@@H]2C[C@H](c3c(O)ccc(Cl)c3Cl)CN12] | <1 | >30 |
| 127 | 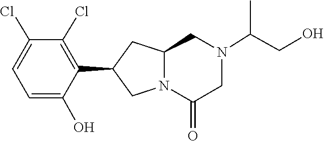 [SMILES: CC(CO)N1CC(=O)N2C[C@@H](c3c(O)ccc(Cl)c3Cl)C[C@H]2C1] | <1 | >30 |
| 128 | 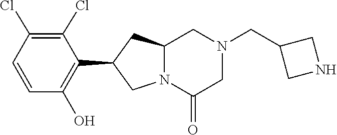 [SMILES: O=C1CN(CC2CNC2)C[C@@H]2C[C@H](c3c(O)ccc(Cl)c3Cl)CN12] | <1 | >30 |
| 129 | 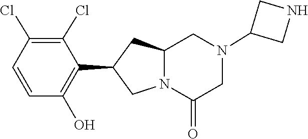 [SMILES: O=C1CN(C2CNC2)C[C@@H]2C[C@H](c3c(O)ccc(Cl)c3Cl)CN12] | <1 | >30 |
| 130 | 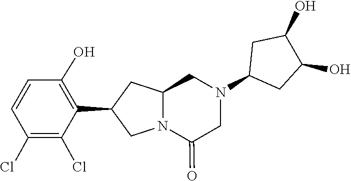 [SMILES: O=C1CN([C@H]2C[C@@H](O)[C@@H](O)C2)C[C@@H]2C[C@H](c3c(O)ccc(Cl)c3Cl)CN12] | <1 | >30 |
| 131 | 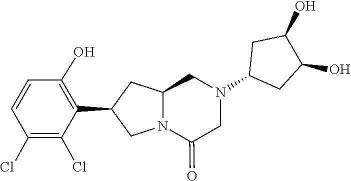 [SMILES: O=C1CN([C@@H]2C[C@@H](O)[C@@H](O)C2)C[C@@H]2C[C@H](c3c(O)ccc(Cl)c3Cl)CN12] | <1 | >30 |
| 132 | 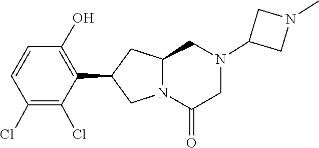 [SMILES: CN1CC(N2CC(=O)N3C[C@@H](c4c(O)ccc(Cl)c4Cl)C[C@H]3C2)C1] | <1 | <30 |
| 133 | 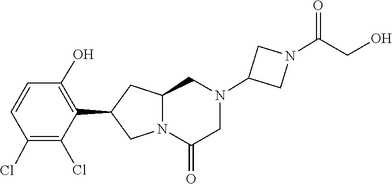 [SMILES: O=C(CO)N1CC(N2CC(=O)N3C[C@@H](c4c(O)ccc(Cl)c4Cl)C[C@H]3C2)C1] | <1 | >30 |
| 134 | 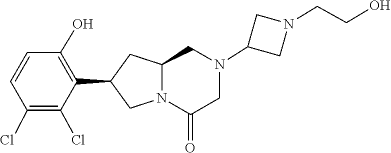 [SMILES: O=C1CN(C2CN(CCO)C2)C[C@@H]2C[C@H](c3c(O)ccc(Cl)c3Cl)CN12] | <1 | >30 |
| 135 | 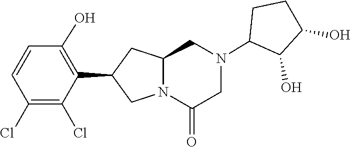 [SMILES: O=C1CN(C2CC[C@H](O)[C@@H]2O)C[C@@H]2C[C@H](c3c(O)ccc(Cl)c3Cl)CN12] | <1 | * |
| 136 | 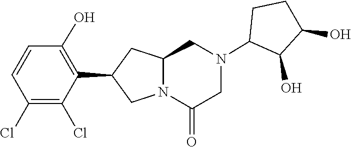 [SMILES: O=C1CN(C2CC[C@@H](O)[C@H]2O)C[C@@H]2C[C@H](c3c(O)ccc(Cl)c3Cl)CN12] | <1 | >30 |
| 137 | 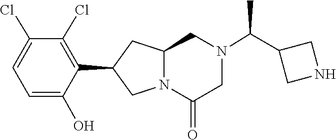 [SMILES: C[C@@H](C1CNC1)N1CC(=O)N2C[C@@H](c3c(O)ccc(Cl)c3Cl)C[C@H]2C1] | <1 | >30 |
| 138 | 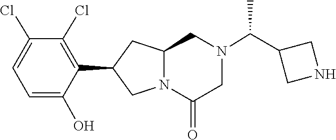 [SMILES: C[C@H](C1CNC1)N1CC(=O)N2C[C@@H](c3c(O)ccc(Cl)c3Cl)C[C@H]2C1] | <1 | >30 |
| 139 | 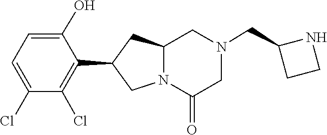 [SMILES: O=C1CN(C[C@@H]2CCN2)C[C@@H]2C[C@H](c3c(O)ccc(Cl)c3Cl)CN12] | <1 | >30 |
| 140 | 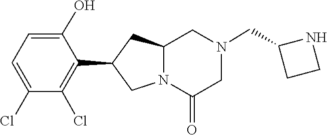 [SMILES: O=C1CN(C[C@H]2CCN2)C[C@@H]2C[C@H](c3c(O)ccc(Cl)c3Cl)CN12] | <1 | >30 |
| 141 | 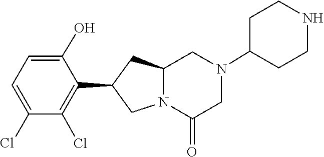 [SMILES: O=C1CN(C2CCNCC2)C[C@@H]2C[C@H](c3c(O)ccc(Cl)c3Cl)CN12] | <1 | >30 |
| 142 | 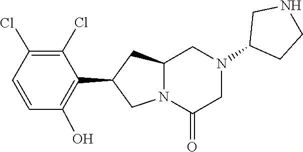 [SMILES: O=C1CN([C@H]2CCNC2)C[C@@H]2C[C@H](c3c(O)ccc(Cl)c3Cl)CN12] | <1 | >30 |
| 143 | 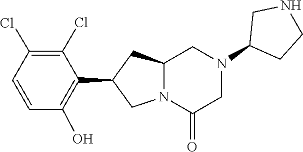 [SMILES: O=C1CN([C@@H]2CCNC2)C[C@@H]2C[C@H](c3c(O)ccc(Cl)c3Cl)CN12] | <1 | >30 |
| 144 | 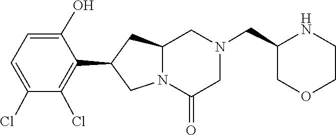 [SMILES: O=C1CN(C[C@@H]2COCCN2)C[C@@H]2C[C@H](c3c(O)ccc(Cl)c3Cl)CN12] | <1 | >30 |
| 145 | 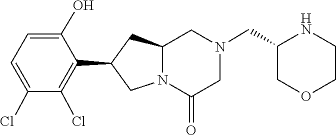 [SMILES: O=C1CN(C[C@H]2COCCN2)C[C@@H]2C[C@H](c3c(O)ccc(Cl)c3Cl)CN12] | <1 | <30 |
| 146 | 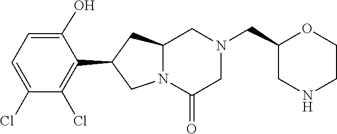 [SMILES: O=C1CN(C[C@@H]2CNCCO2)C[C@@H]2C[C@H](c3c(O)ccc(Cl)c3Cl)CN12] | <1 | >30 |
| 147 | 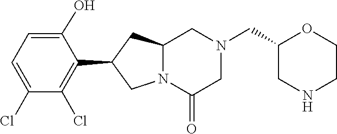 [SMILES: O=C1CN(C[C@H]2CNCCO2)C[C@@H]2C[C@H](c3c(O)ccc(Cl)c3Cl)CN12] | <1 | >30 |
| 148 | 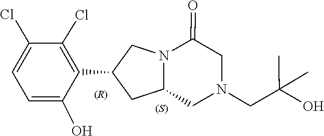 | <1 | >30 |
| 149 | 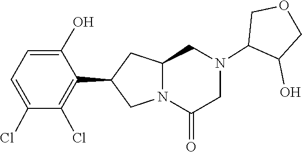 [SMILES: O=C1CN(C2COCC2O)C[C@@H]2C[C@H](c3c(O)ccc(Cl)c3Cl)CN12] | <1 | >30 |
| 150 | 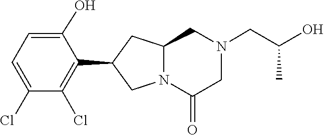 [SMILES: C[C@@H](O)CN1CC(=O)N2C[C@@H](c3c(O)ccc(Cl)c3Cl)C[C@H]2C1] | <1 | >30 |
| 151 | 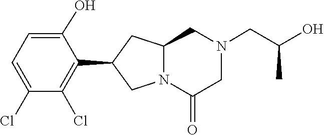 [SMILES: C[C@H](O)CN1CC(=O)N2C[C@@H](c3c(O)ccc(Cl)c3Cl)C[C@H]2C1] | <1 | >30 |
| 152 | 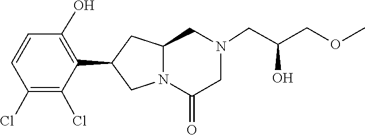 [SMILES: COC[C@@H](O)CN1CC(=O)N2C[C@@H](c3c(O)ccc(Cl)c3Cl)C[C@H]2C1] | <1 | <30 |
| 153 | 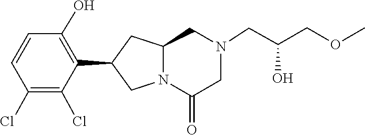 [SMILES: COC[C@H](O)CN1CC(=O)N2C[C@@H](c3c(O)ccc(Cl)c3Cl)C[C@H]2C1] | <1 | <30 |
| 154 | 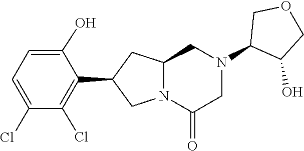 [SMILES: O=C1CN([C@H]2COC[C@@H]2O)C[C@@H]2C[C@H](c3c(O)ccc(Cl)c3Cl)CN12] | <1 | >30 |
| 155 | 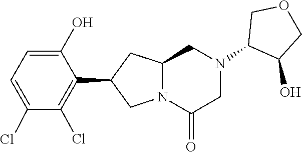 [SMILES: O=C1CN([C@@H]2COC[C@H]2O)C[C@@H]2C[C@H](c3c(O)ccc(Cl)c3Cl)CN12] | <1 | >30 |
| 156 | 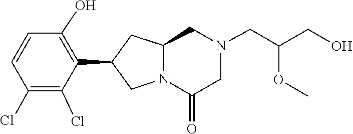 [SMILES: COC(CO)CN1CC(=O)N2C[C@@H](c3c(O)ccc(Cl)c3Cl)C[C@H]2C1] | <1 | >30 |
| 157 | 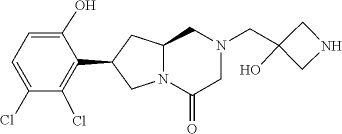 [SMILES: O=C1CN(CC2(O)CNC2)C[C@@H]2C[C@H](c3c(O)ccc(Cl)c3Cl)CN12] | <1 | >30 |
| 158 | 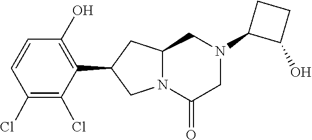 [SMILES: O=C1CN([C@H]2CC[C@@H]2O)C[C@@H]2C[C@H](c3c(O)ccc(Cl)c3Cl)CN12] | <1 | >30 |
| 159 | 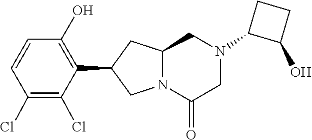 [SMILES: O=C1CN([C@@H]2CC[C@H]2O)C[C@@H]2C[C@H](c3c(O)ccc(Cl)c3Cl)CN12] | <1 | >30 |
| 160 | 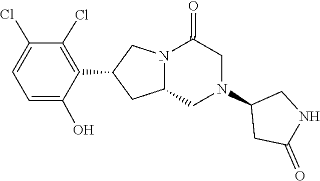 [SMILES: O=C1C[C@@H](N2CC(=O)N3C[C@@H](c4c(O)ccc(Cl)c4Cl)C[C@H]3C2)CN1] | <1 | >30 |
| 161 | 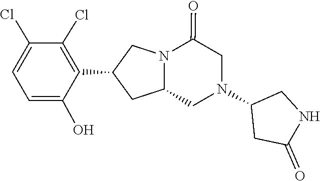 [SMILES: O=C1C[C@H](N2CC(=O)N3C[C@@H](c4c(O)ccc(Cl)c4Cl)C[C@H]3C2)CN1] | <1 | >30 |
| 162 | 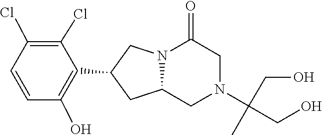 [SMILES: CC(CO)(CO)N1CC(=O)N2C[C@@H](c3c(O)ccc(Cl)c3Cl)C[C@H]2C1] | <1 | >30 |
| 163 | 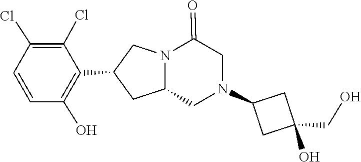 [SMILES: O=C1CN([C@H]2C[C@](O)(CO)C2)C[C@@H]2C[C@H](c3c(O)ccc(Cl)c3Cl)CN12] | <1 | >30 |
| 164 | 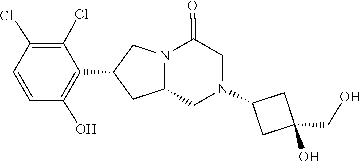 [SMILES: O=C1CN([C@H]2C[C@@](O)(CO)C2)C[C@@H]2C[C@H](c3c(O)ccc(Cl)c3Cl)CN12] | <1 | >30 |
| 165 | 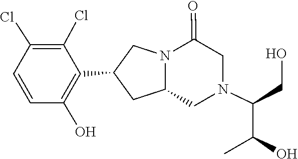 [SMILES: C[C@H](O)[C@H](CO)N1CC(=O)N2C[C@@H](c3c(O)ccc(Cl)c3Cl)C[C@H]2C1] | <1 | >30 |
| 166 | 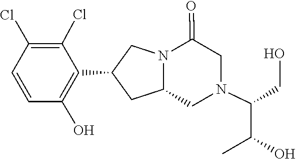 [SMILES: C[C@@H](O)[C@@H](CO)N1CC(=O)N2C[C@@H](c3c(O)ccc(Cl)c3Cl)C[C@H]2C1] | <1 | >30 |
| 167 | 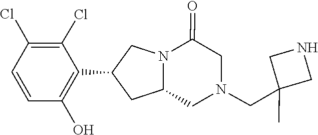 [SMILES: CC1(CN2CC(=O)N3C[C@@H](c4c(O)ccc(Cl)c4Cl)C[C@H]3C2)CNC1] | <1 | >30 |
| 168 | 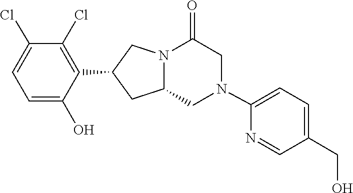 [SMILES: O=C1CN(c2ccc(CO)cn2)C[C@@H]2C[C@H](c3c(O)ccc(Cl)c3Cl)CN12] | <1 | * |
| 169 | 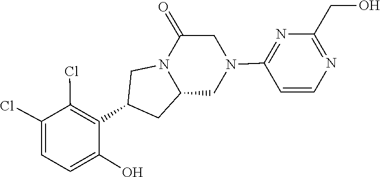 [SMILES: O=C1CN(c2ccnc(CO)n2)C[C@@H]2C[C@H](c3c(O)ccc(Cl)c3Cl)CN12] | <1 | >30 |
| 170 | 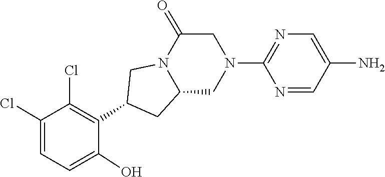 [SMILES: Nc1cnc(N2CC(=O)N3C[C@@H](c4c(O)ccc(Cl)c4Cl)C[C@H]3C2)nc1] | <1 | * |
| 171 | 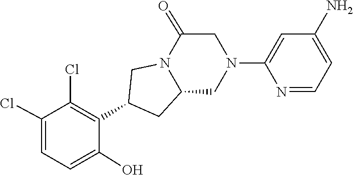 [SMILES: Nc1ccnc(N2CC(=O)N3C[C@@H](c4c(O)ccc(Cl)c4Cl)C[C@H]3C2)c1] | <1 | <10 |
| 172 | 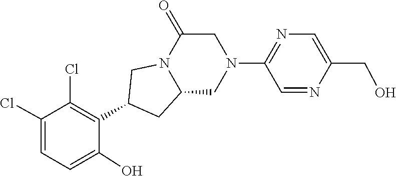 [SMILES: O=C1CN(c2cnc(CO)cn2)C[C@@H]2C[C@H](c3c(O)ccc(Cl)c3Cl)CN12] | <1 | <30 |
| 173 | 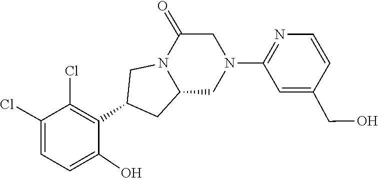 [SMILES: O=C1CN(c2cc(CO)ccn2)C[C@@H]2C[C@H](c3c(O)ccc(Cl)c3Cl)CN12] | <1 | <30 |
| 174 | 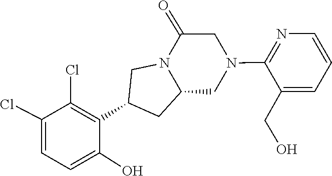 [SMILES: O=C1CN(c2ncccc2CO)C[C@@H]2C[C@H](c3c(O)ccc(Cl)c3Cl)CN12] | <10 | * |
| 175 | 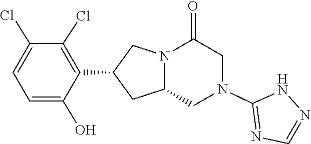 [SMILES: O=C1CN(c2ncn[nH]2)C[C@@H]2C[C@H](c3c(O)ccc(Cl)c3Cl)CN12] | <1 | >30 |
| 176 | 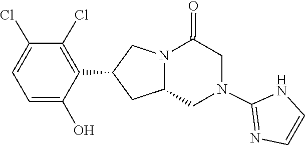 [SMILES: O=C1CN(c2ncc[nH]2)C[C@@H]2C[C@H](c3c(O)ccc(Cl)c3Cl)CN12] | <1 | <30 |
| 180 | 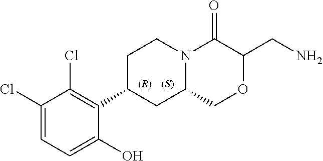 | <1 | <30 |
| 181 | 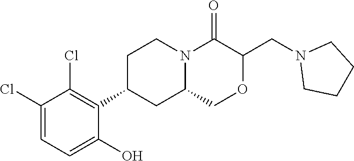 | <1 | <10 |
| 182 | 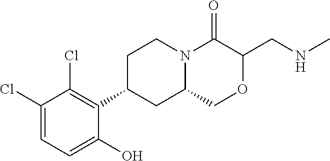 | <1 | <30 |
| 183 | 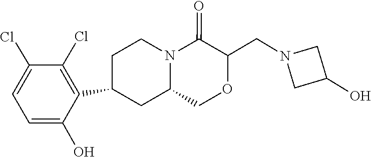 | <1 | <30 |
| 184 | 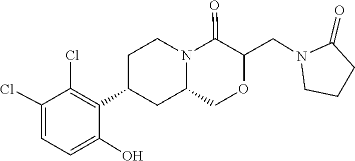 | <10 | * |
| 185 | 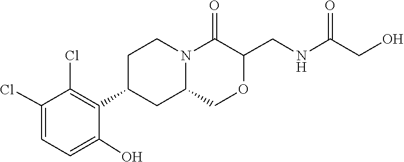 | <1 | >30 |
| 186 | 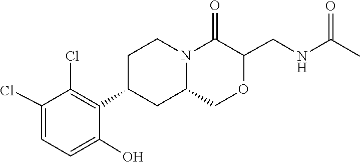 | <1 | * |
| 187 | 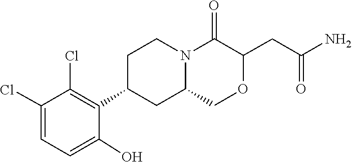 | <1 | >30 |
| 188 | 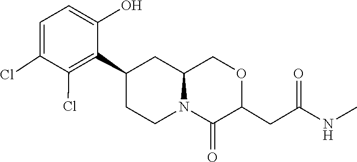 | <1 | >30 |
| 189 | 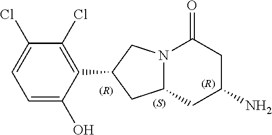 | <1 | >30 |
| 190 | 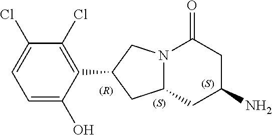 | <1 | >30 |
| 191 | 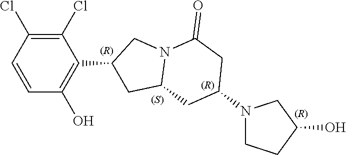 | <1 | <30 |
| 192 | 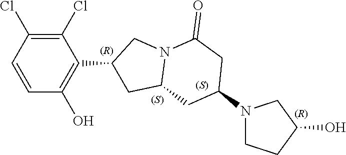 | <1 | >30 |
| 193 | 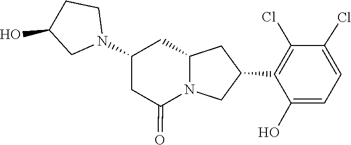 [SMILES: O=C1C[C@H](N2CC[C@H](O)C2)C[C@@H]2C[C@H](c3c(O)ccc(Cl)c3Cl)CN12] | <1 | >30 |
| 194 | 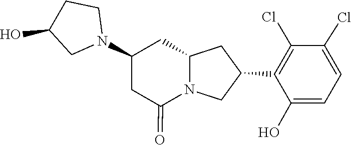 [SMILES: O=C1C[C@@H](N2CC[C@H](O)C2)C[C@@H]2C[C@H](c3c(O)ccc(Cl)c3Cl)CN12] | <1 | >30 |
| 195 | 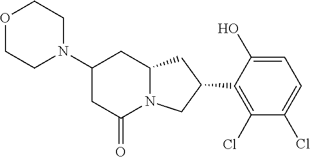 [SMILES: O=C1CC(N2CCOCC2)C[C@@H]2C[C@H](c3c(O)ccc(Cl)c3Cl)CN12] | <1 | <30 |
| 196 | 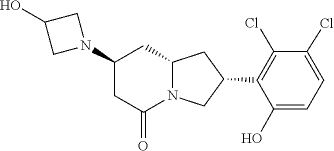 [SMILES: O=C1C[C@@H](N2CC(O)C2)C[C@@H]2C[C@H](c3c(O)ccc(Cl)c3Cl)CN12] | <1 | >30 |
| 197 | 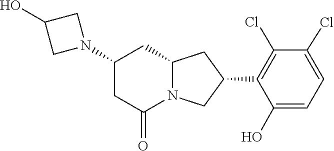 [SMILES: O=C1C[C@H](N2CC(O)C2)C[C@@H]2C[C@H](c3c(O)ccc(Cl)c3Cl)CN12] | <1 | >30 |
| 198 | 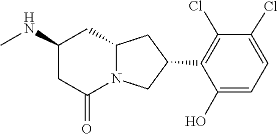 [SMILES: CN[C@@H]1CC(=O)N2C[C@@H](c3c(O)ccc(Cl)c3Cl)C[C@H]2C1] | <1 | >30 |
| 199 | 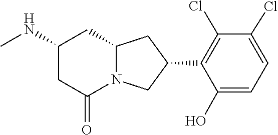 [SMILES: CN[C@H]1CC(=O)N2C[C@@H](c3c(O)ccc(Cl)c3Cl)C[C@H]2C1] | <1 | >30 |
| 200 | 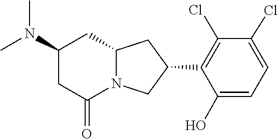 [SMILES: CN(C)[C@@H]1CC(=O)N2C[C@@H](c3c(O)ccc(Cl)c3Cl)C[C@H]2C1] | <1 | <10 |
| 201 | 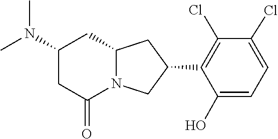 [SMILES: CN(C)[C@H]1CC(=O)N2C[C@@H](c3c(O)ccc(Cl)c3Cl)C[C@H]2C1] | <1 | <10 |
| 202 | 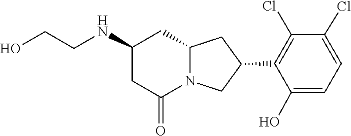 [SMILES: O=C1C[C@@H](NCCO)C[C@@H]2C[C@H](c3c(O)ccc(Cl)c3Cl)CN12] | <1 | >30 |
| 203 | 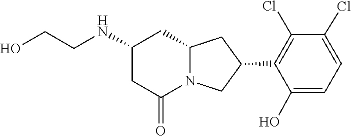 [SMILES: O=C1C[C@H](NCCO)C[C@@H]2C[C@H](c3c(O)ccc(Cl)c3Cl)CN12] | <1 | >30 |
| 204 | 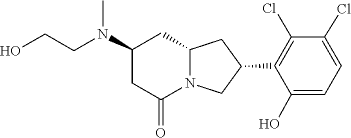 [SMILES: CN(CCO)[C@@H]1CC(=O)N2C[C@@H](c3c(O)ccc(Cl)c3Cl)C[C@H]2C1] | <1 | >30 |
| 205 | 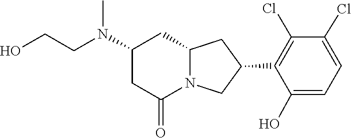 [SMILES: CN(CCO)[C@H]1CC(=O)N2C[C@@H](c3c(O)ccc(Cl)c3Cl)C[C@H]2C1] | <1 | >30 |
| 206 | 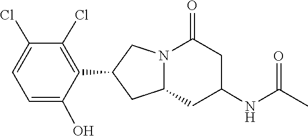 | <1 | >30 |
| 207 | 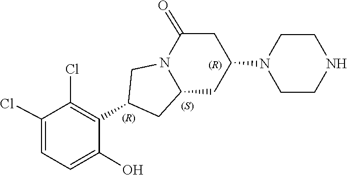 | <1 | >30 |
| 208 | 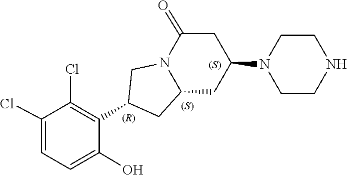 | <1 | <10 |
| 213 | 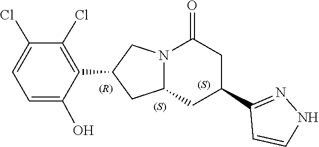 | <1 | * |
| 214 | 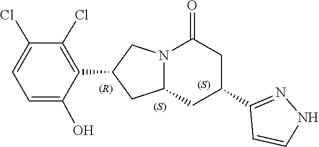 | <10 | * |
| 215 | 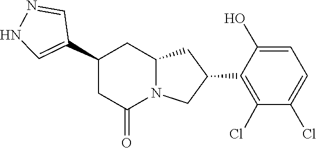 [SMILES: O=C1C[C@@H](c2cn[nH]c2)C[C@@H]2C[C@H](c3c(O)ccc(Cl)c3Cl)CN12] | <1 | >30 |
| 216 | 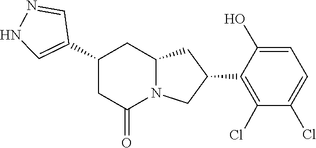 [SMILES: O=C1C[C@H](c2cn[nH]c2)C[C@@H]2C[C@H](c3c(O)ccc(Cl)c3Cl)CN12] | <1 | <10 |
| 217 | 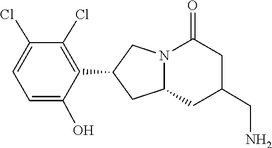 | <1 | >30 |
| 218 | 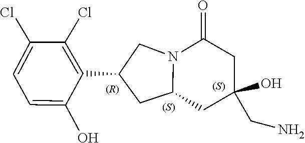 | <1 | >30 |
| 219 | 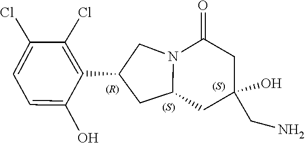 | <1 | >30 |
| 220 | 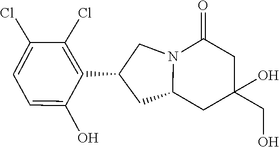 [SMILES: O=C1CC(O)(CO)C[C@@H]2C[C@H](c3c(O)ccc(Cl)c3Cl)CN12] | <1 | >30 |
| 223 | 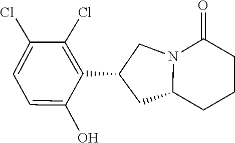 [SMILES: O=C1CCC[C@@H]2C[C@H](c3c(O)ccc(Cl)c3Cl)CN12] | <1 | >30 |
| 224 | 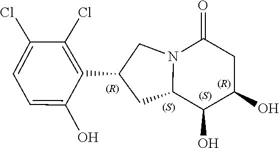 | <1 | * |
| 225 | 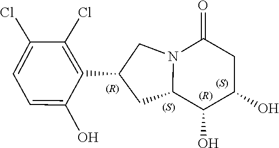 | <1 | >30 |
| 226 | 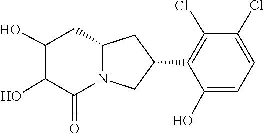 | <1 | >30 |
| 227 | 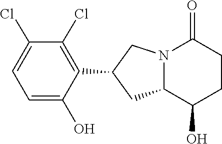 [SMILES: O=C1CC[C@@H](O)[C@@H]2C[C@H](c3c(O)ccc(Cl)c3Cl)CN12] | <1 | * |
| 228 | 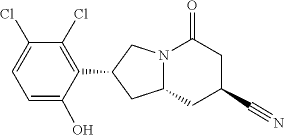 [SMILES: N#C[C@@H]1CC(=O)N2C[C@@H](c3c(O)ccc(Cl)c3Cl)C[C@H]2C1] | <1 | * |
| 229 | 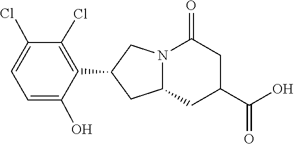 | <1 | * |
| 230 | 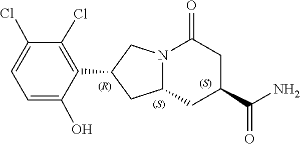 | <1 | * |
| 231 | 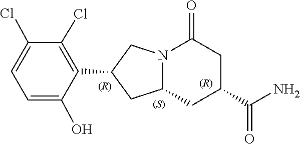 | <1 | >30 |
| 232 | 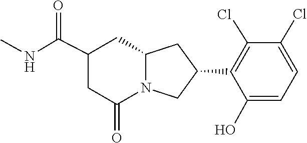 | <1 | >30 |
| 233 | 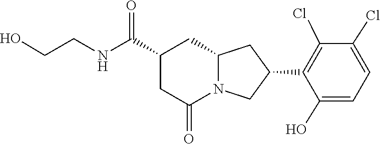 [SMILES: O=C(NCCO)[C@H]1CC(=O)N2C[C@@H](c3c(O)ccc(Cl)c3Cl)C[C@H]2C1] | <1 | * |
| 234 | 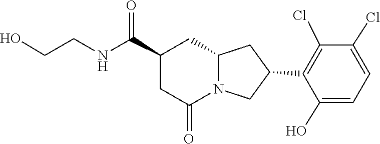 [SMILES: O=C(NCCO)[C@@H]1CC(=O)N2C[C@@H](c3c(O)ccc(Cl)c3Cl)C[C@H]2C1] | <1 | >30 |
| 235 | 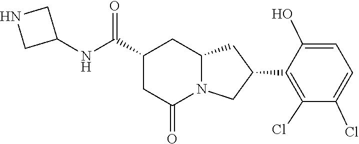 [SMILES: O=C(NC1CNC1)[C@H]1CC(=O)N2C[C@@H](c3c(O)ccc(Cl)c3Cl)C[C@H]2C1] | <1 | >30 |
| 236 | 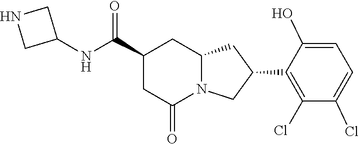 [SMILES: O=C(NC1CNC1)[C@@H]1CC(=O)N2C[C@@H](c3c(O)ccc(Cl)c3Cl)C[C@H]2C1] | <1 | >30 |
| 237 | 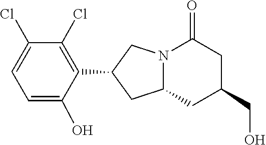 [SMILES: O=C1C[C@@H](CO)C[C@@H]2C[C@H](c3c(O)ccc(Cl)c3Cl)CN12] | <1 | >30 |
| 238 | 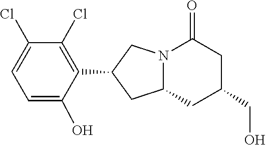 [SMILES: O=C1C[C@H](CO)C[C@@H]2C[C@H](c3c(O)ccc(Cl)c3Cl)CN12] | <1 | >30 |
| 239 | 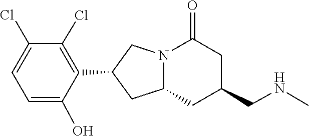 [SMILES: CNC[C@@H]1CC(=O)N2C[C@@H](c3c(O)ccc(Cl)c3Cl)C[C@H]2C1] | <1 | >30 |
| 240 | 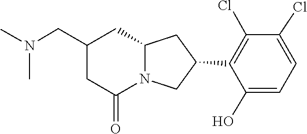 | <1 | <10 |
| 241 | 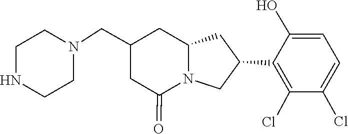 [SMILES: O=C1CC(CN2CCNCC2)C[C@@H]2C[C@H](c3c(O)ccc(Cl)c3Cl)CN12] | <1 | >30 |
| 242 | 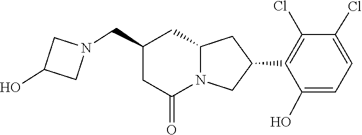 [SMILES: O=C1C[C@@H](CN2CC(O)C2)C[C@@H]2C[C@H](c3c(O)ccc(Cl)c3Cl)CN12] | <1 | >30 |
| 243 | 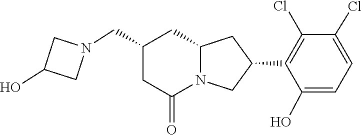 [SMILES: O=C1C[C@H](CN2CC(O)C2)C[C@@H]2C[C@H](c3c(O)ccc(Cl)c3Cl)CN12] | <1 | <30 |
| 244 | 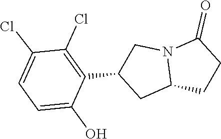 [SMILES: O=C1CC[C@@H]2C[C@H](c3c(O)ccc(Cl)c3Cl)CN12] | <1 | * |
| 245 | 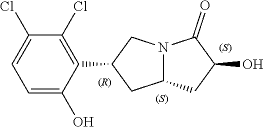 | <1 | * |
| 246 | 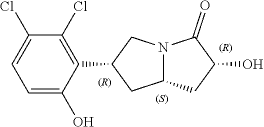 | <1 | * |
| 247 | 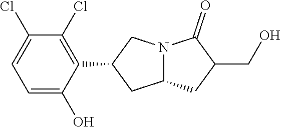 | <1 | * |
| 248 | 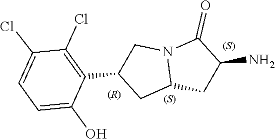 | <1 | <10 |
| 249 | 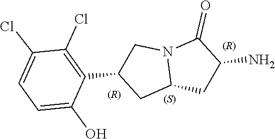 | <1 | * |
| 250 | 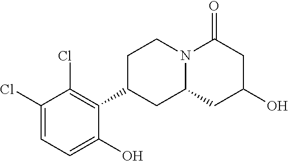 | <1 | >30 |
| 251 | 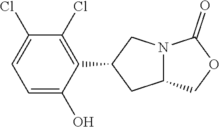 [SMILES: O=C1OC[C@@H]2C[C@H](c3c(O)ccc(Cl)c3Cl)CN12] | <1 | <30 |
| 252 | 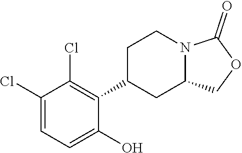 | <1 | * |
| 253 | 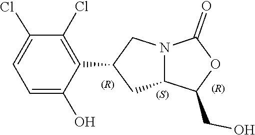 | <1 | >30 |
| 254 | 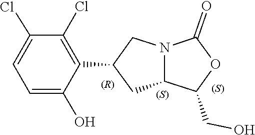 | <1 | >30 |
| 255 | 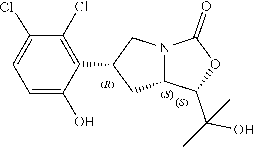 | <1 | * |
| 256 | 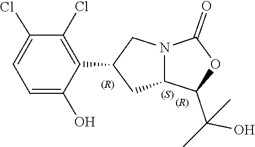 | <1 | >30 |
| 257 | 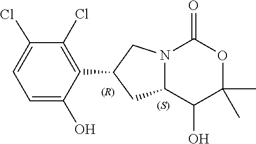 | <1 | >30 |
| 258 | 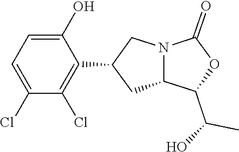 [SMILES: C[C@H](O)[C@H]1OC(=O)N2C[C@@H](c3c(O)ccc(Cl)c3Cl)C[C@@H]12] | <1 | >30 |
| 259 | 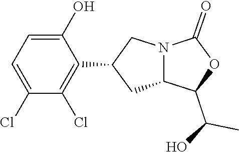 [SMILES: C[C@@H](O)[C@@H]1OC(=O)N2C[C@@H](c3c(O)ccc(Cl)c3Cl)C[C@@H]12] | <1 | >30 |
| 260 | 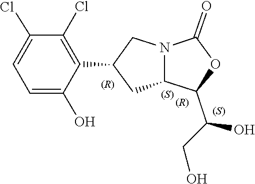 | <1 | >30 |
| 261 | 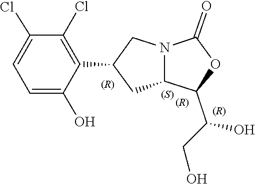 | <1 | >30 |
| 262 | 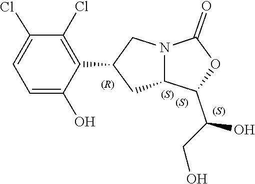 | <1 | >30 |
| 263 | 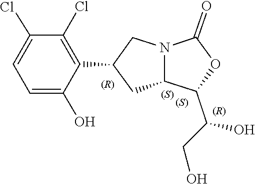 | <1 | >30 |
| 264 | 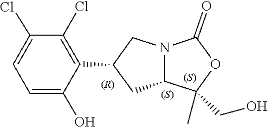 | <1 | >30 |
| 265 | 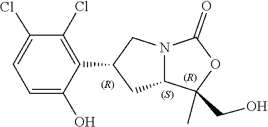 | <1 | >30 |
| 266 | 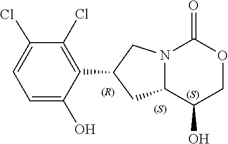 | <1 | >30 |
| 267 | 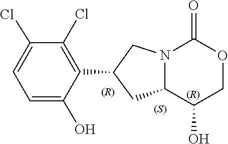 | <1 | >30 |
| 268 | 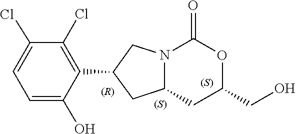 | <1 | >30 |
| 269 | 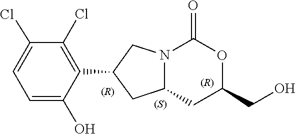 | <1 | >30 |
| 270 | 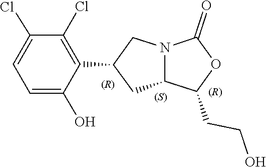 | <1 | <30 |
| 271 | 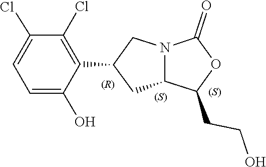 | <1 | <30 |
| 272 | 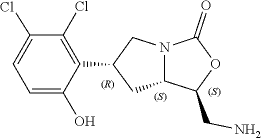 | <1 | <30 |
| 273 | 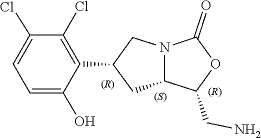 | <1 | >30 |
| 274 | 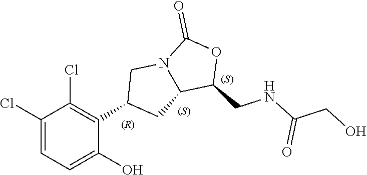 | <1 | >30 |
| 275 | 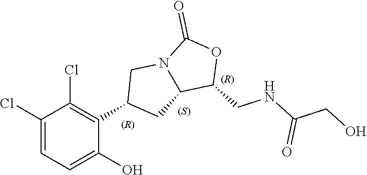 | <1 | >30 |
| 276 | 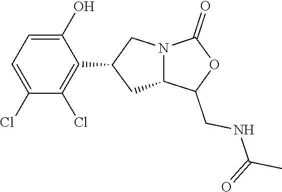 [SMILES: CC(=O)NCC1OC(=O)N2C[C@@H](c3c(O)ccc(Cl)c3Cl)C[C@@H]12] | <1 | >30 |
| 277 | 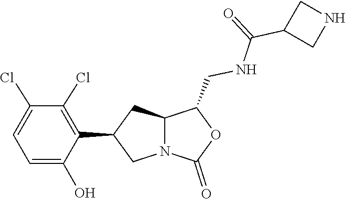 [SMILES: O=C(NC[C@@H]1OC(=O)N2C[C@@H](c3c(O)ccc(Cl)c3Cl)C[C@@H]12)C1CNC1] | <1 | >30 |
| 278 | 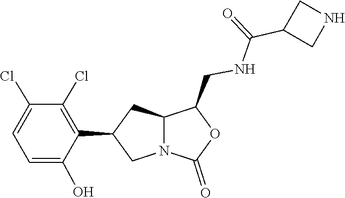 [SMILES: O=C(NC[C@H]1OC(=O)N2C[C@@H](c3c(O)ccc(Cl)c3Cl)C[C@@H]12)C1CNC1] | <1 | >30 |
| 279 | 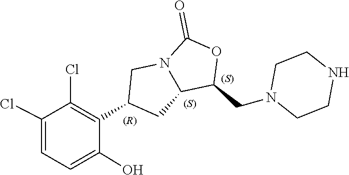 | <1 | <30 |
| 280 | 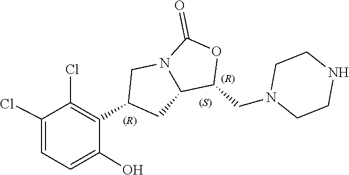 | <1 | >30 |
| 281 | 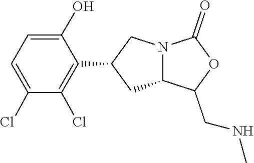 [SMILES: CNCC1OC(=O)N2C[C@@H](c3c(O)ccc(Cl)c3Cl)C[C@@H]12] | <1 | <30 |
| 282 | 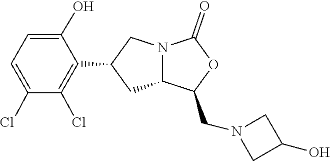 [SMILES: O=C1O[C@@H](CN2CC(O)C2)[C@@H]2C[C@H](c3c(O)ccc(Cl)c3Cl)CN12] | <1 | <30 |
| 283 | 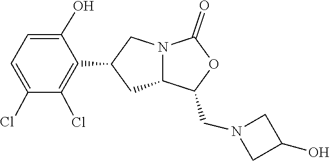 [SMILES: O=C1O[C@H](CN2CC(O)C2)[C@@H]2C[C@H](c3c(O)ccc(Cl)c3Cl)CN12] | <1 | >30 |
| 284 | 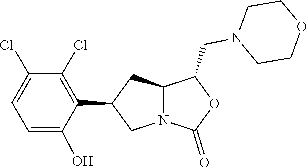 [SMILES: O=C1O[C@@H](CN2CCOCC2)[C@@H]2C[C@H](c3c(O)ccc(Cl)c3Cl)CN12] | <1 | >30 |
| 285 | 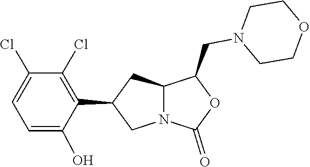 [SMILES: O=C1O[C@H](CN2CCOCC2)[C@@H]2C[C@H](c3c(O)ccc(Cl)c3Cl)CN12] | <1 | >30 |
| 286 | 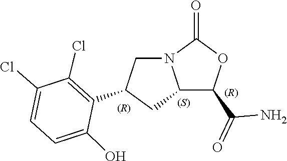 | <1 | >30 |
| 287 | 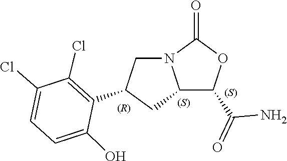 | <1 | >30 |
| 288 | 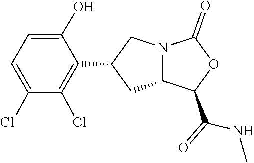 [SMILES: CNC(=O)[C@@H]1OC(=O)N2C[C@@H](c3c(O)ccc(Cl)c3Cl)C[C@@H]12] | <1 | >30 |
| 289 | 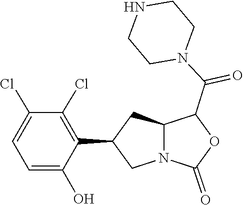 [SMILES: O=C(C1OC(=O)N2C[C@@H](c3c(O)ccc(Cl)c3Cl)C[C@@H]12)N1CCNCC1] | <1 | >30 |
| 290 | 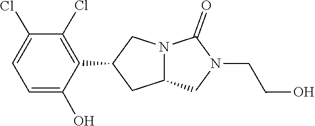 [SMILES: O=C1N(CCO)C[C@@H]2C[C@H](c3c(O)ccc(Cl)c3Cl)CN12] | <10 | * |
| 291 | 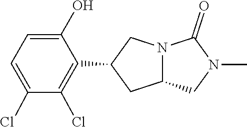 [SMILES: CN1C[C@@H]2C[C@H](c3c(O)ccc(Cl)c3Cl)CN2C1=O] | <10 | * |
| 292 | 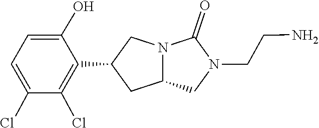 [SMILES: NCCN1C[C@@H]2C[C@H](c3c(O)ccc(Cl)c3Cl)CN2C1=O] | <1 | <30 |
| 293 | 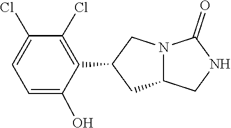 [SMILES: O=C1NC[C@@H]2C[C@H](c3c(O)ccc(Cl)c3Cl)CN12] | <1 | >30 |

*Not Tested.

1. A compound of Formula I or a pharmaceutically acceptable salt thereof, 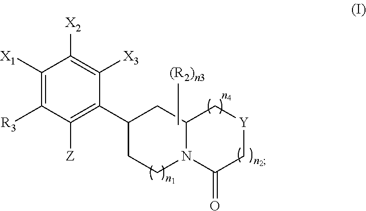
where
Y is C(R₂)₂, NR₁, or O;
Z is ORₐ;
X₁ is H, halogen, or alkyl;
X₂ is H, halogen, CN, alkyl, cycloalkyl, halogenated cycloalkyl, or halogenated alkyl;
X₃ is H, halogen, halogenated alkyl, or alkyl;
or alternatively X₁ and X₂ and the carbon atoms they are connected to taken together form an optionally substituted 5- or 6-membered aryl;
or alternatively X₂ and X₃ and the carbon atoms they are connected to taken together form an optionally substituted 5- or 6-membered aryl;
each occurrence of R₁ is independently H, alkyl, alkenyl, cycloalkyl, heteroalkyl, cycloheteroalkyl, aryl, heteroaryl, (CR₆R₇)ₙ₆ORₐ, (CR₆R₇)ₙ₆N(Rₐ)₂, (C═O)Rₐ, (C═O)ORₐ, (CR₆R₇)ₙ₆(C═O)NRₐR_{b}, SO₂Rₐ or (CR₆R₇)ₙ₆-heterocycle;
each occurrence of R₂ is independently H, halogen, CN, alkyl, cycloalkyl, heteroalkyl, cycloheteroalkyl, (CR₆R₇)ₙ₆ORₐ, (CR₆R₇)ₙ₆-heterocycle, (C═O)ORₐ, (CR₆R₇)ₙ₆NRₐ(C═O)Rₐ, (CR₆R₇)ₙ₆N(Rₐ)₂, NRₐ(CR₆R₇)ₙ₆ORₐ, (C═O)NRₐ(CR₆R₇)ₙ₆ORₐ, (C═O)Rₐ, (CR₆R₇)ₙ₆(C═O)NRₐR_{b}, aryl, or heteroaryl, wherein each R₂ may be attached to any one of the carbon ring atoms of 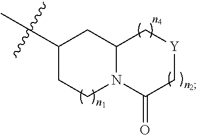
R₃ is H, alkyl, or halogen;
each occurrence of R₆ and R₇ are independently H, alkyl, cycloalkyl, optionally substituted aryl, or optionally substituted heteroaryl;
each occurrence of Rₐ and R_{b} are independently H, alkyl, alkenyl, cycloalkyl, saturated heterocycle, aryl, or heteroaryl; or alternatively Rₐ and R_{b} together with the nitrogen atom that they are connected to form an optionally substituted heterocycle;
the heterocycle comprises 1-3 heteroatoms each selected from the group consisting of N, O, and S;
the alkyl, cycloalkyl, heteroalkyl, cycloheteroalkyl, heterocycle, aryl, and heteroaryl in X₁, X₂, X₃, R₁, R₂, R₃, R₆, R₇, Rₐ, and R_{b}, where applicable, are each independently and optionally substituted by 1-4 substituents each independently selected from the group consisting of alkyl, cycloalkyl, halogenated alkyl, halogenated cycloalkyl, halogen, CN, R₈, OR₈, —(CH₂)₁₋₂OR₈, N(R₈)₂, (C═O)R₈, (C═O)N(R₈)₂, NR₈(C═O)R₈, and oxo where valence permits;
each occurrence of R₈ is independently H, alkyl, cycloalkyl, or a heterocycle optionally substituted by alkyl; or alternatively the two R₈ groups together with the nitrogen atom that they are connected to form a heterocycle optionally substituted by alkyl and comprising the nitrogen atom and 0-3 additional heteroatoms each selected from the group consisting of N, O, and S;
n₁ is an integer from 0-1;
n₂ is an integer from 0-2;
n₃ is an integer from 0-3;
n₄ is an integer from 1 to 2; and
n₆ is an integer from 0-3.

2. The compound of claim 1, wherein the structural moiety 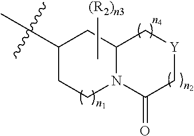
has the structure of

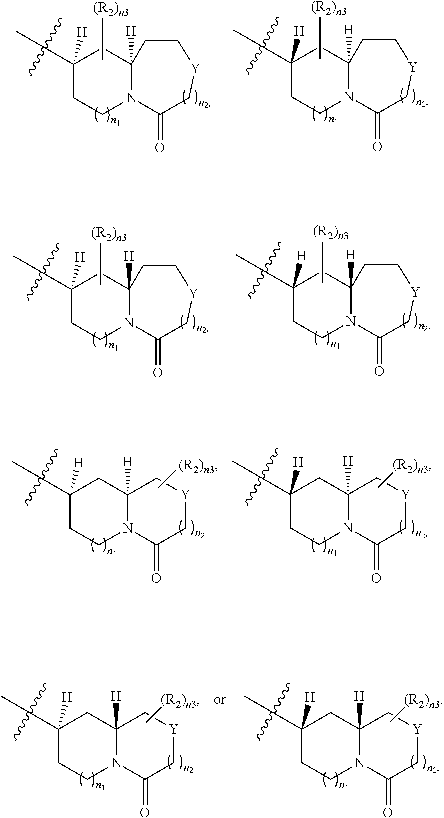

3. The compound of claim 1, wherein the structural moiety 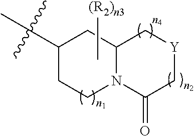
has the structure of

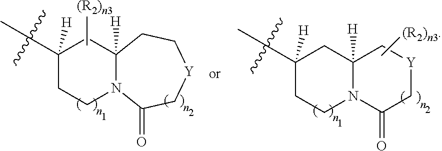

4. The compound of claim 1, wherein the structural moiety 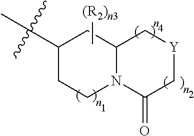
has the structure of

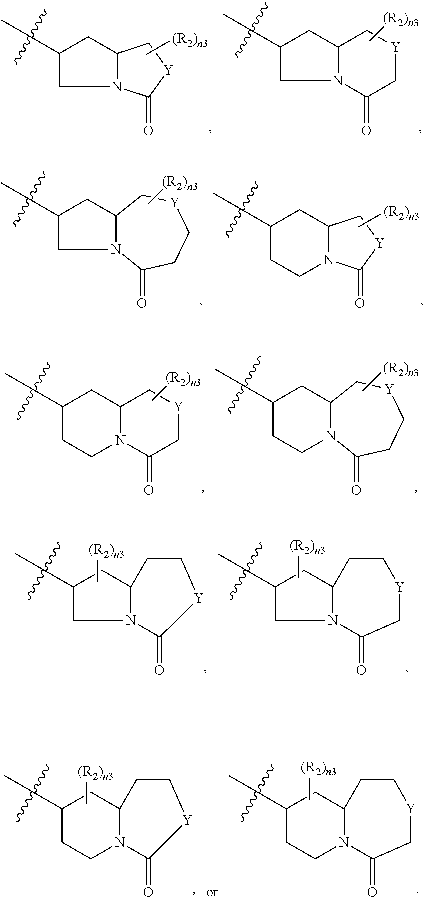

5. The compound of claim 4, wherein the structural moiety 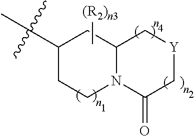
has the structure of

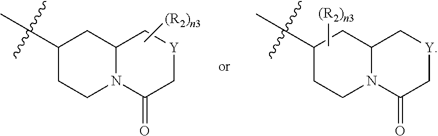

6. The compound of claim 1, wherein the structural moiety 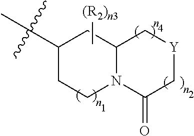
has the structure of

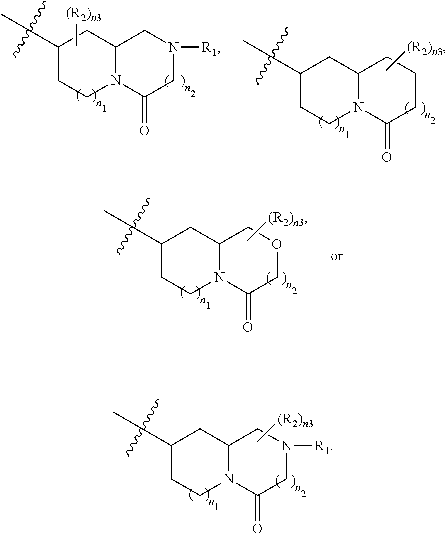

7. The compound of claim 1, wherein the structural moiety 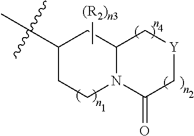
has the structure of

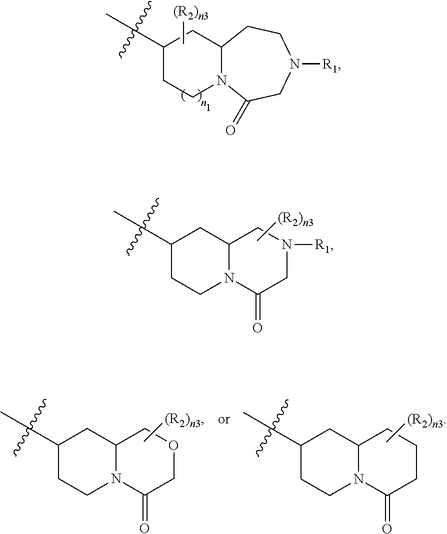

8. The compound of claim 6, wherein the structural moiety 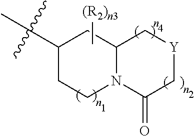
has the structure of

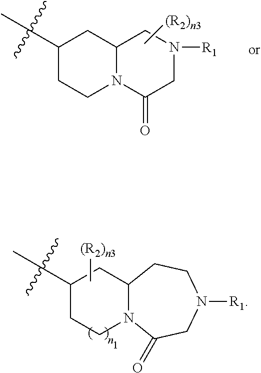

9. The compound of claim 1, wherein R₁ is H, alkyl, alkenyl, cycloalkyl, heteroalkyl, or cycloheteroalkyl.

10. The compound of claim 1, wherein R₁ is aryl or heteroaryl.

11. The compound of claim 1, wherein R₁ is (C═O)Rₐ, (C═O)ORₐ, SO₂Rₐ, (CR₆R₇)ₙ₆ORₐ, (CR₆R₇)ₙ₆N(Rₐ)₂, (CR₆R₇)ₙ₆(C═O)NRₐR_{b}, or (CR₆R₇)ₙ₆-heterocycle.

12. The compound of claim 1, wherein R₁ is (C═O)Rₐ.

13. The compound of claim 11, wherein Rₐ and R_{b} are each independently H, alkyl, or alkyl substituted by one or more OR₈.

14. The compound of claim 13, wherein R₈ is H or alkyl.

15. The compound of claim 1, wherein R₁ is selected from the group consisting of H, —CH₃, —(CH₂)₂OH, —(CH₂)₂NH₂, —CONH₂, —CONHMe, —CONMe₂, —CONEt₂, SO₂Me, and SO₂Et.

16. The compound of claim 1, wherein R₁ is selected from the group consisting of H,

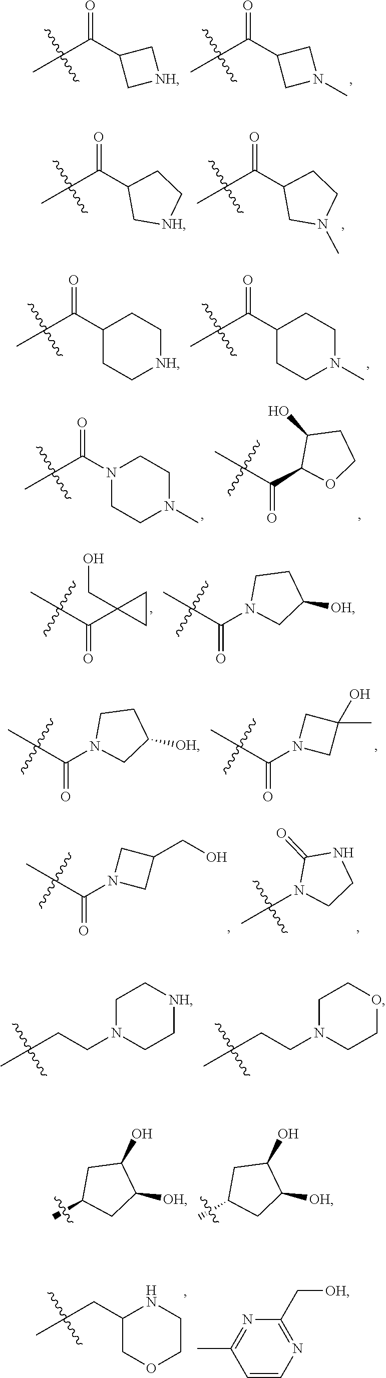

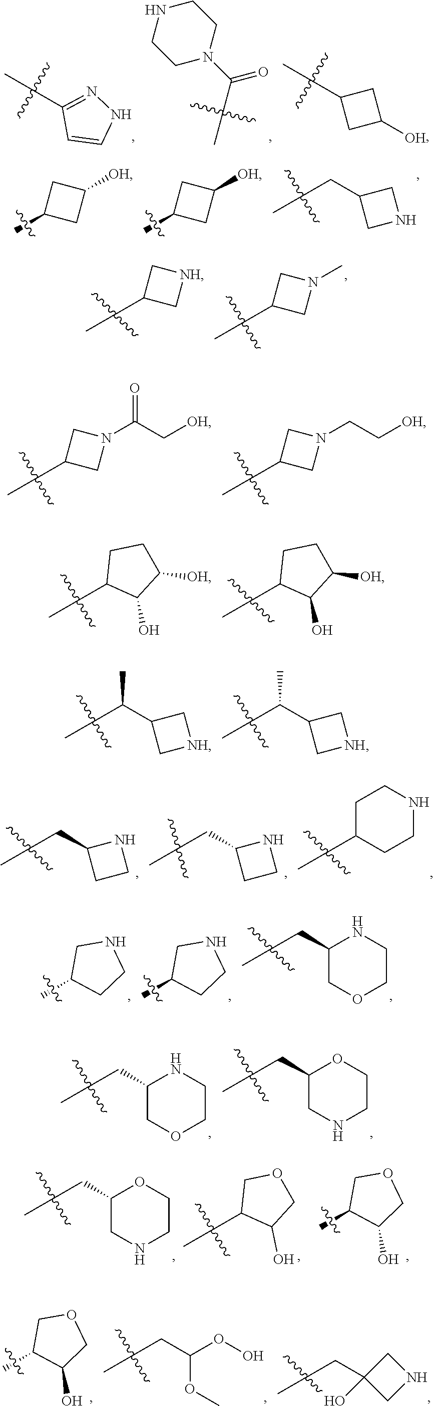

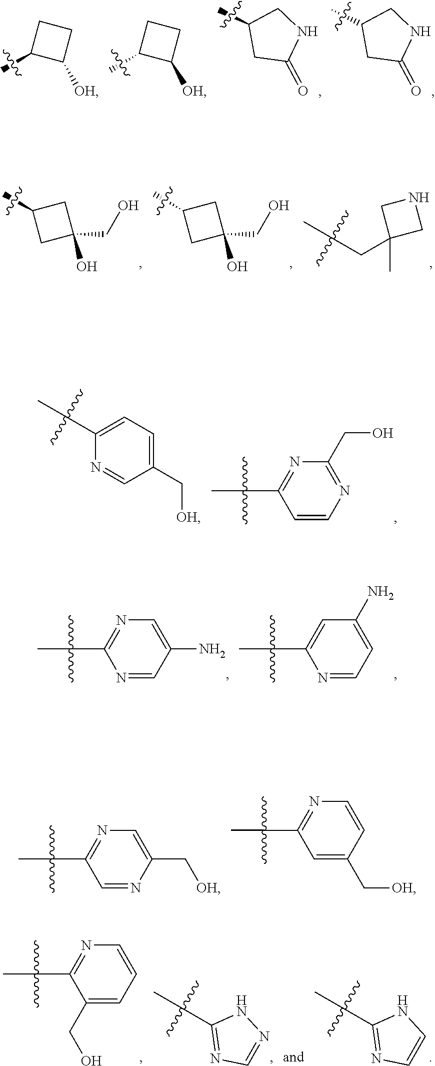

17. The compound of claim 1, wherein R₁ is selected from the group consisting of

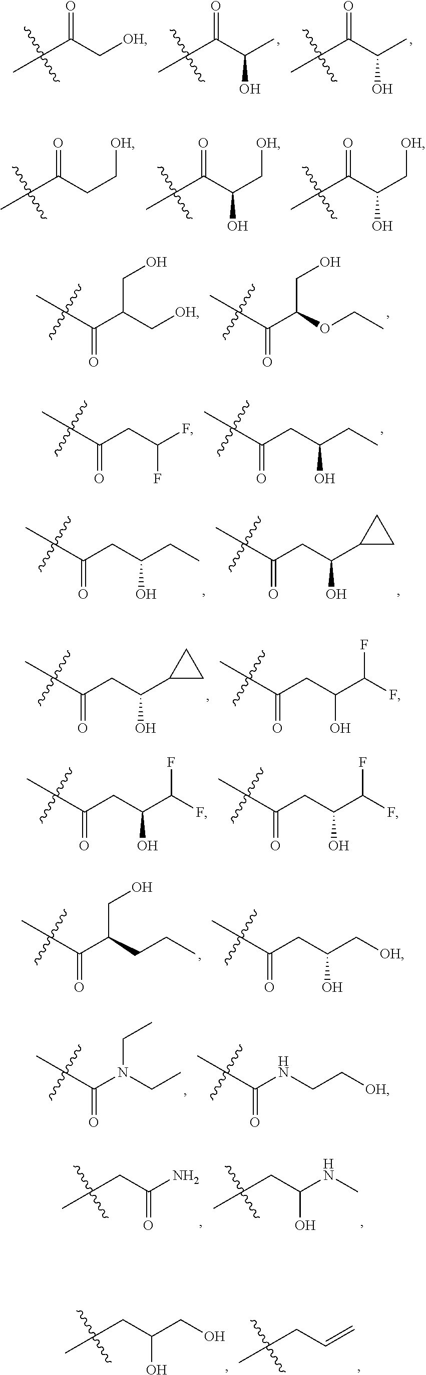

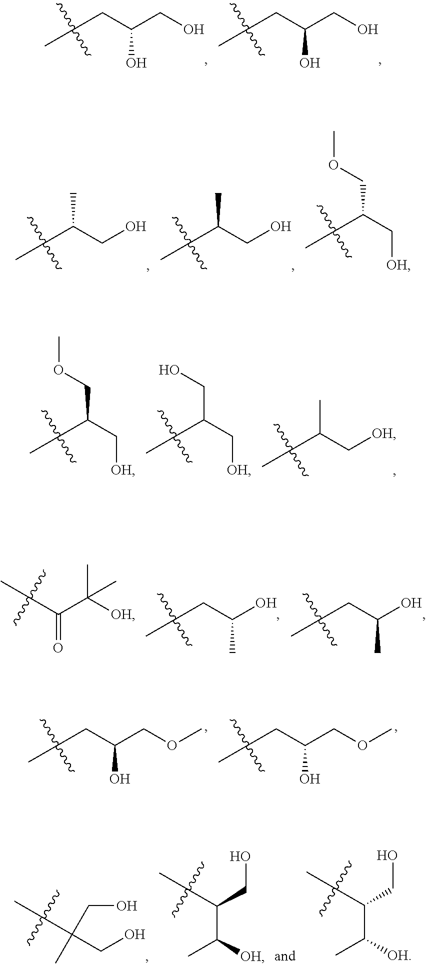

18. The compound of claim 1, wherein at least one occurrence of R₂ is H, halogen, CN, alkyl, heteroalkyl, cycloalkyl, cycloheteroalkyl, ORₐ, N(R₁)₂, (C═O)Rₐ, (C═O)NRₐR_{b}, aryl, or heteroaryl.

19. The compound of claim 1, wherein at least one occurrence of R₂ is (CR₆R₇)ₙ₆ORₐ, (CR₆R₇)ₙ₆-heterocycle, (C═O)Rₐ, (C═O)ORₐ, (CR₆R₇)ₙ₆NRₐ(C═O)Rₐ, (CR₆R₇)ₙ₆N(Rₐ)₂, NRₐ(CR₆R₇)ₙ₆ORₐ, (C═O)NRₐ(CR₆R₇)ₙ₆ORₐ, or (CR₆R₇)ₙ₆(C═O)NRₐR_{b}.

20. The compound of claim 1, wherein at least one occurrence of R₂ is CH₃, —CH₂—OH, —CH₂—CH₂—OH, —CH(OH)—CH₃, —CH₂—NH₂,

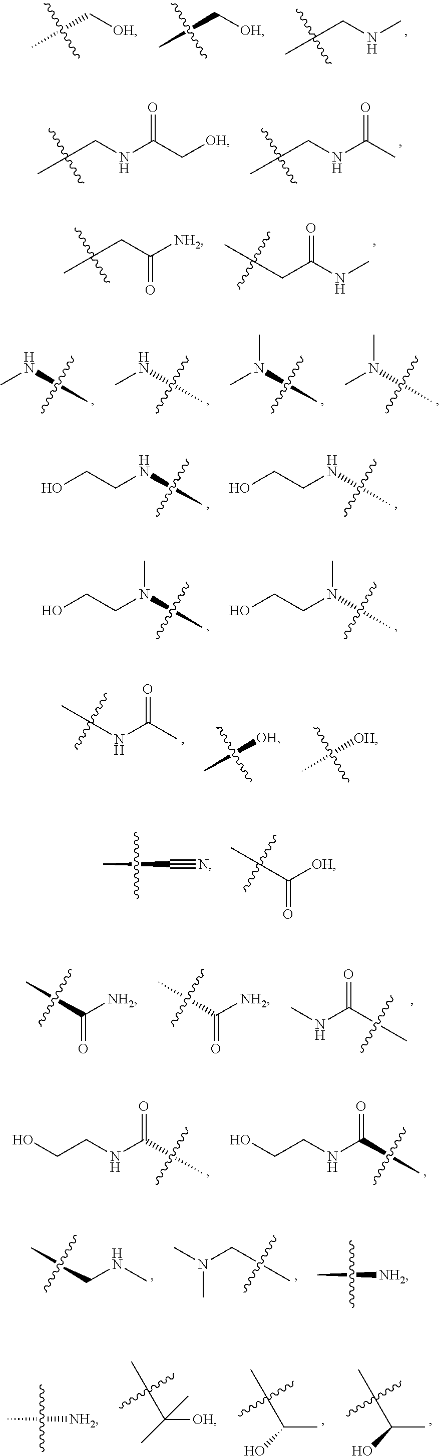

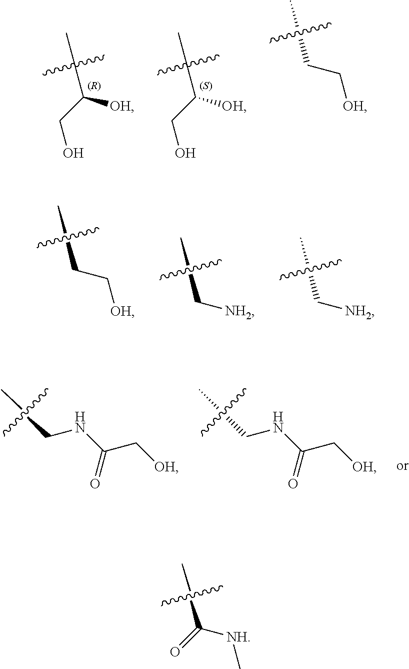

21. The compound of claim 1, wherein at least one occurrence of R₂ is heteroalkyl, cycloheteroalkyl,

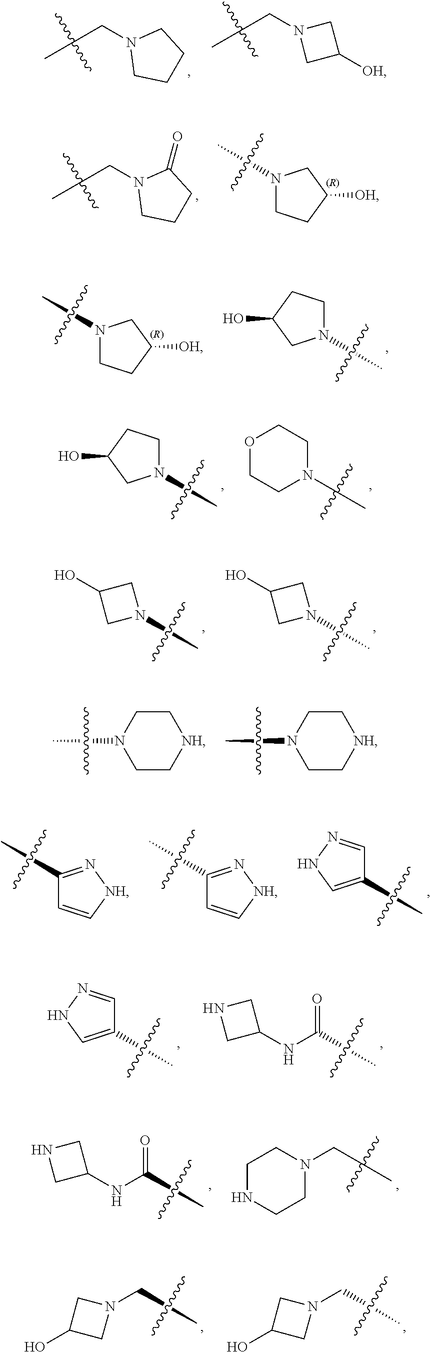

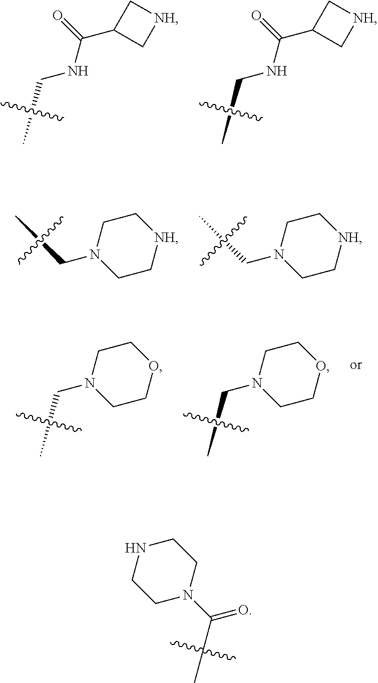

22. The compound of claim 1, wherein n₁ is 0.

23. The compound of claim 1, wherein n₁ is 1.

24. The compound of claim 1, wherein n₂ is 0 or 1.

25. The compound of claim 1, wherein n₃ is 0, 1, or 2.

26. The compound of claim 1, wherein n₄ is 1.

27. The compound of claim 1, wherein n₆ is 0, 1, or 2.

28. The compound of claim 1, wherein Z is OH, OMe, OEt, OPr, O-i-Pr, O-t-Bu, O-iso-Bu, O-sec-Bu, or OBu.

29. The compound of claim 28, wherein Z is OH, OMe, or OEt.

30. The compound of claim 29, wherein Z is OH.

31. The compound of claim 1, wherein X₁ is H, halogen, Me, or Et.

32. The compound of claim 31, wherein X₁ is H, F, Cl, Br, or Me.

33. The compound of claim 32, wherein X₁ is H or Cl.

34. The compound of claim 1, wherein X₂ is H, halogen, fluorinated alkyl, or alkyl.

35. The compound of claim 34, wherein X₂ is H, F, Cl, Br, Me, CF₂H, CF₂Cl, or CF₃.

36. The compound of claim 35, wherein X₂ is H or Cl.

37. The compound of claim 1, wherein X₃ is H, F, Cl, Br, Me, CF₂H, CF₂Cl, or CF₃.

38. The compound of claim 37, wherein X₃ is H or Cl.

39. The compound of claim 1, wherein R₃ is H.

40. The compound of claim 1, wherein R₃ is alkyl.

41. The compound of claim 1, wherein R₃ is halogen.

42. The compound of claim 1, wherein R₃ is H, F, Cl, or Me.

43. The compound of claim 1, wherein the structural moiety 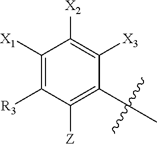
has the structure of

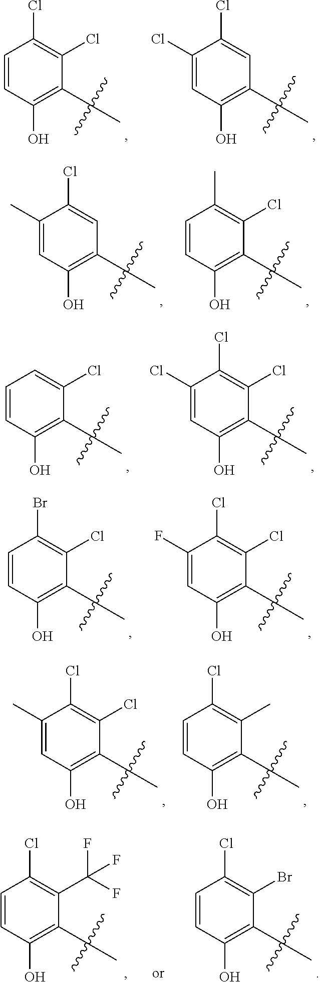

44. The compound of claim 1, wherein the compound has a structure of Formula II′ or II: 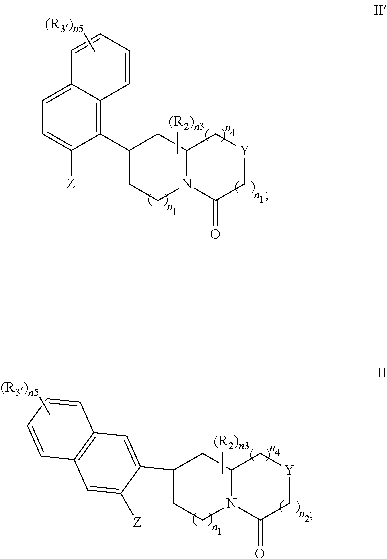
wherein R_{3′} is independently H, halogen, or alkyl; and
n₅ is an integer from 0-3.

45. The compound of claim 44, wherein n₅ is 0, 1, or 2.

46. The compound of claim 44, wherein n₅ is 0.

47. The compound of claim 44, wherein R_{3′} is H or alkyl.

48. The compound of claim 44, wherein R_{3′} is halogen.

49. The compound of claim 44, wherein Z is OH, OMe, OEt, OPr, O-i-Pr, O-t-Bu, O-iso-Bu, O-sec-Bu, or OBu.

50. The compound of claim 49, wherein Z is OH, OMe, or OEt.

51. The compound of claim 50, wherein Z is OH.

52. The compound of claim 1, wherein at least one occurrence of Rₐ or R_{b} is independently H, alkyl, cycloalkyl, saturated heterocycle, aryl, or heteroaryl.

53. The compound of claim 52, wherein at least one occurrence of Rₐ or R_{b} is independently H, Me, Et, Pr, or a heterocycle selected from the group consisting of 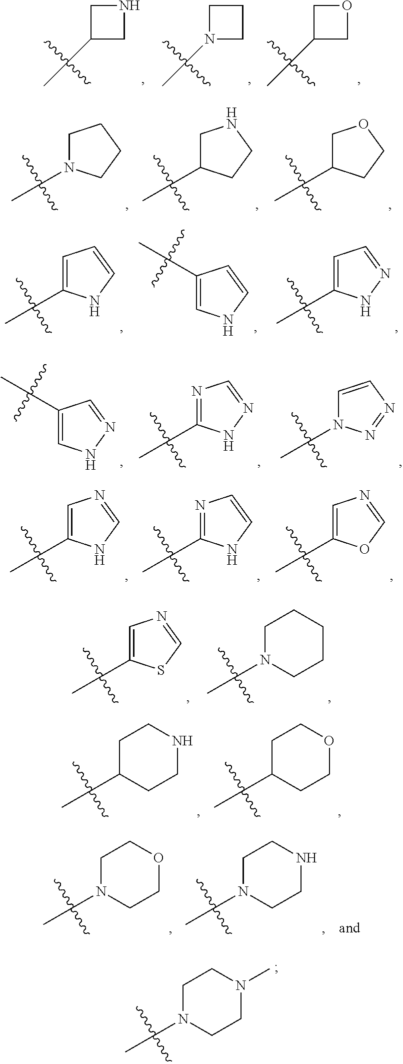
wherein the heterocycle is optionally substituted by alkyl, OH, oxo, or (C═O)C₁₋₄alkyl where valence permits.

54. The compound of claim 1, wherein Rₐ and R_{b} together with the nitrogen atom that they are connected to form an optionally substituted heterocycle comprising the nitrogen atom and 0-3 additional heteroatoms each selected from the group consisting of N, O, and S.

55. The compound of claim 1, wherein the heterocycle is selected from the group consisting of

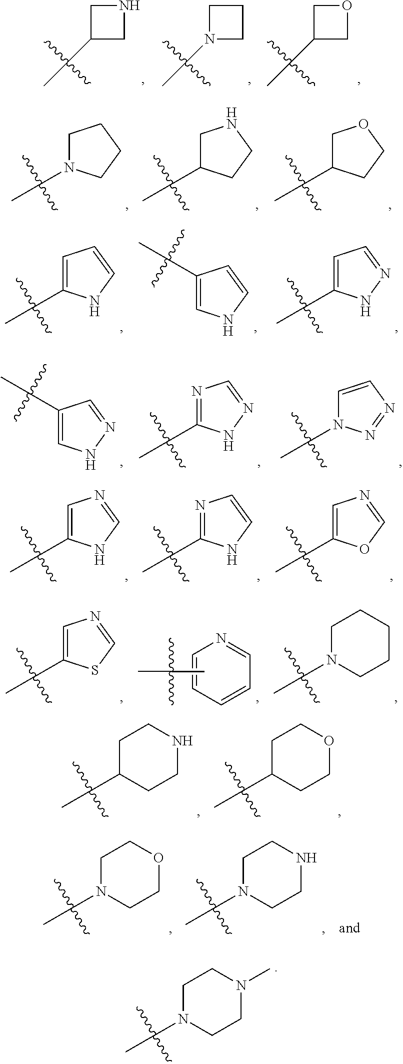

56. The compound of claim 1, wherein the compound is selected from the group consisting of compounds 1-62 as shown in Table 4.

57. The compound of claim 1, wherein the compound is selected from the group consisting of compounds 63-78, 83-85, 87-88, 90-94, 96-97, 99-104, 109-176, 180-208, 213-220, 223-293 as shown in Table 5.

58. A pharmaceutical composition comprising at least one compound according to claim 1 or a pharmaceutically acceptable salt thereof and a pharmaceutically acceptable carrier or diluent.

59. A method of treating a condition in a mammalian species in need thereof, comprising administering to the mammalian species a therapeutically effective amount of at least one compound according to claim 1 or a pharmaceutically acceptable salt thereof, wherein the condition is selected from the group consisting of cancer, an immunological disorder, a Central Nerve System (CNS) disorder, an inflammatory disorder, a gastroenterological disorder, a metabolic disorder, a cardiovascular disorder, and a kidney disease.

60. The method of claim 59, wherein the immunological disorder is transplant rejection or an autoimmune disease.

61. The method of claim 60, wherein the autoimmune disease is rheumatoid arthritis, multiple sclerosis, systemic lupus erythematosus, or type I diabetes mellitus.

62. The method of claim 59, wherein the central nervous system disorder is Alzheimer's disease.

63. The method of claim 59, wherein the inflammatory disorder is an inflammatory skin condition, arthritis, psoriasis, spondylitis, parodontitits, or an inflammatory neuropathy.

64. The method of claim 59, wherein the gastroenterological disorder is an inflammatory bowel disease.

65. The method of claim 59, wherein the metabolic disorder is obesity or type II diabetes mellitus.

66. The method of claim 59, wherein the cardiovascular disorder is an ischemic stroke.

67. The method of claim 59, wherein the kidney disease is chronic kidney disease, nephritis, or chronic renal failure.

68. The method of claim 59, wherein the condition is selected from the group consisting of cancer, transplant rejection, rheumatoid arthritis, multiple sclerosis, systemic lupus erythematosus, type I diabetes mellitus, Alzheimer's disease, inflammatory skin condition, inflammatory neuropathy, psoriasis, spondylitis, parodontitis, Crohn's disease, ulcerative colitis, obesity, type II diabetes mellitus, ischemic stroke, chronic kidney disease, nephritis, chronic renal failure, and a combination thereof.

69. The method of claim 59, wherein the mammalian species is human.

70. A method of blocking Kv1.3 potassium channel in a mammalian species in need thereof, comprising administering to the mammalian species a therapeutically effective amount of at least one compound according to claim 1 or a pharmaceutically acceptable salt thereof.

71. The method of claim 70, wherein the mammalian species is human.
